(19) United States
(12) Patent Application Publication
(10) Pub. No.: US 2026/0109732 A1
(43) Pub. Date: Apr. 23, 2026

(54) LIPID AMINES

(71) Applicant: ModernaTX, Inc., Cambridge, MA (US)

(72) Inventors: Mohindra Seepersaud, Acton, MA (US); Matthew Crawford, Cambridge, MA (US); Daniel Tate, Somerville, MA (US); Mark Cornebise, Arlington, MA (US)

(21) Appl. No.: 18/705,135

(22) PCT Filed: Oct. 28, 2022
(86) PCT No.: PCT/US2022/048223

§ 371 Date: Apr. 26, 2024

Related U.S. Application Data
(60) Provisional application No. 63/273,421, filed on Oct. 29, 2021.
(60) Provisional application No. 63/308,180, filed on Feb. 9, 2022.
(60) Provisional application No. 63/395,243, filed on Aug. 4, 2022.

(51) Int. Cl. C07J 41/00; A61K 9/51; A61K 31/7088; C07J 43/00

(52) U.S. Cl. CPC C07J 41/0055; A61K 9/5123; A61K 9/5192; A61K 31/7088; C07J 43/003

(57) ABSTRACT

Provided are lipid amine compounds which are useful in the preparation of lipid nanoparticle compositions for delivery of therapeutic or prophylactic payload into cells.

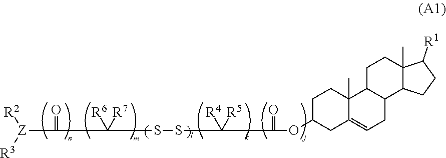

### CROSS-REFERENCE TO RELATED APPLICATIONS

This application claims the benefit of the filing date of U.S. Provisional Application No. 63/273,421, filed Oct. 29, 2021, U.S. Provisional Application No. 63/308,180, filed Feb. 9, 2022, and U.S. Provisional Application No. 63/395,243, filed Aug. 4, 2022, the contents of which are incorporated by reference in their entirety herein.

### FIELD

Provided are lipid amine compounds which are useful in the preparation of lipid nanoparticle compositions for delivery of therapeutic or prophylactic payload into cells.

### BACKGROUND

The delivery of biologically active payloads, such as nucleic acids and proteins, to cells has the potential to be used to treat a variety of diseases and/or conditions. However, effective targeted delivery of such payloads represents a continuing medical challenge. In particular, the delivery of nucleic acids to cells is made difficult by the relative instability and low cell permeability of such species.

Lipid nanoparticles provided an effective transport vehicle for the payloads into cells and intracellular compartments, but improvements in safety, efficacy, and specificity are still needed. Thus, there exists a need to develop lipid nanoparticle compositions to facilitate the delivery of therapeutics and prophylactics, such as nucleic acids, into cells.

### SUMMARY

Provided herein is a lipid amine having the structure of Formula A1:

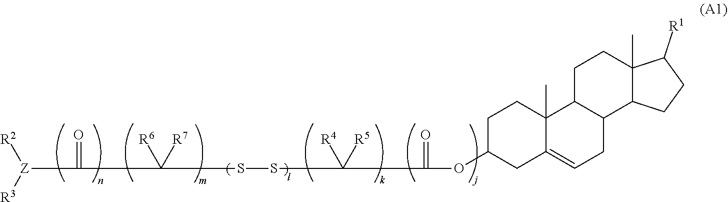

or a salt thereof, wherein constituent members are defined herein.

Also provided herein is a lipid nanoparticle composition comprising a lipid amine of Formula A1, or a salt thereof.

Also provided herein is a pharmaceutical composition comprising a pharmaceutically acceptable carrier and a lipid nanoparticle composition comprising a lipid amine of Formula A1, or a salt thereof.

Also provided herein is a method of delivering a payload into a cell comprising contacting the cell with a lipid nanoparticle composition described herein.

Also provided herein is a method of delivering a therapeutic or prophylactic payload to a patient comprising administering a lipid nanoparticle composition described herein to the patient.

Also provided herein is a process of preparing a lipid nanoparticle composition, the process comprising contacting a lipid nanoparticle core with a lipid amine compound of Formula A1, or a salt thereof.

Also provided herein is a product of any of the processes described herein.

Each of the limitations can encompass various embodiments. It is, therefore, anticipated that each of the limitations involving any one element or combinations of elements can be included in each aspect described. This invention is not limited in its application to the details of construction and the arrangement of components set forth in the following description or illustrated in the drawings. Other embodiments and of being practiced or of being carried out in various ways are possible.

### DETAILED DESCRIPTION

Provided herein is a lipid amine having the structure of Formula A1:

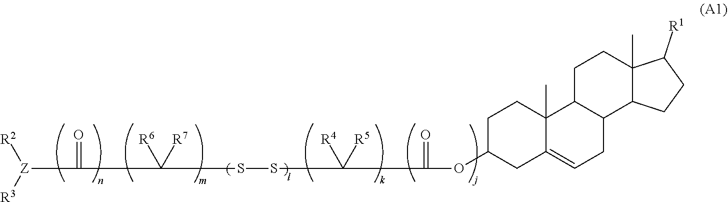

or a salt thereof, wherein:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₂₋₂₀ alkyl are optionally replaced with O;
(iii) 1, 2, 3, or 4 non-terminal carbons of the C₂₋₂₀ alkyl are optionally replaced with NR¹⁰;
(iv) 1, 2, 3, or 4 non-terminal carbons of the C₂₋₂₀ alkyl are optionally replaced with C(═O); and
(v) 1, 2, 3, or 4 non-terminal carbons of the C₂₋₂₀ alkyl are optionally replaced with CR^{a}R^{b} wherein R^{a} and R^{b} together with the C atom to which they are attached form a C₃₋₆ cycloalkyl group;
wherein R² and R³ are the same or different;
   or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo;
or R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo;
R⁴, R⁵, R⁶, and R⁷ are each independently selected from H, halo, and C₁₋₄ alkyl;
or R⁴ and R⁵ together with the carbon atom to which they are attached form a C₃₋₇ cycloalkyl group;
or R⁶ and R⁷ together with the carbon atom to which they are attached form a C₃₋₇ cycloalkyl group;
R⁸, R⁹, and R¹⁰ are each independently selected from H and C₁₋₄ alkyl;
j is 0 or 1;
k is 0, 1, 2, 3, 4, 5, or 6;
1 is 0 or 1;
m is 0, 1, 2, 3, 4, 5, or 6; and
n is 0 or 1;
   wherein when j is 0, then 1 is 1,
wherein j and 1 are not both 0.

Provided herein is a lipid amine having the structure of Formula A1:

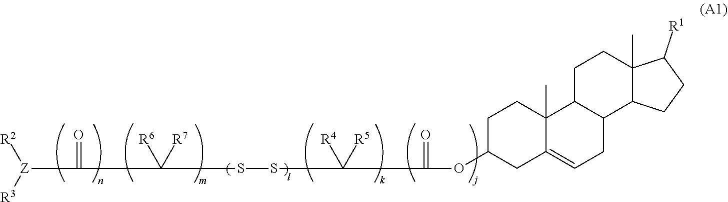

or a salt thereof, wherein:
Z is N or CH;
R¹ is C₁₋₁₄ alkyl, C₁₋₁₄ alkenyl, or C₁₋₁₄ hydroxyalkyl;
R² and R³ are C₂₋₂₀ alkyl, wherein:
(i) the C₂₋₂₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₂₋₂₀ alkyl are optionally replaced with O; and
(iii) 1, 2, 3, or 4 non-terminal carbons of the C₂₋₂₀ alkyl are optionally replaced with NR¹⁰;
   wherein R² and R³ are the same or different;
or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR₈R₉, OH, and halo;
   or R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo;
   R⁴, R⁵, R⁶, and R⁷ are each independently selected from H, halo, and C₁₋₄ alkyl;
   or R⁴ and R⁵ together with the carbon atom to which they are attached form a C₃₋₇ cycloalkyl group;
   or R⁶ and R⁷ together with the carbon atom to which they are attached form a C₃₋₇ cycloalkyl group;
   R⁸, R⁹, and R¹⁰ are each independently selected from H and C₁₋₄ alkyl;
   j is 0 or 1;
   k is 0, 1, 2, 3, 4, 5, or 6;
   1 is 0 or 1;
   m is 0, 1, 2, 3, 4, 5, or 6; and
   n is 0 or 1;
      wherein when j is 0, then 1 is 1,
      wherein j and 1 are not both 0.

In some embodiments, the compound is other than:

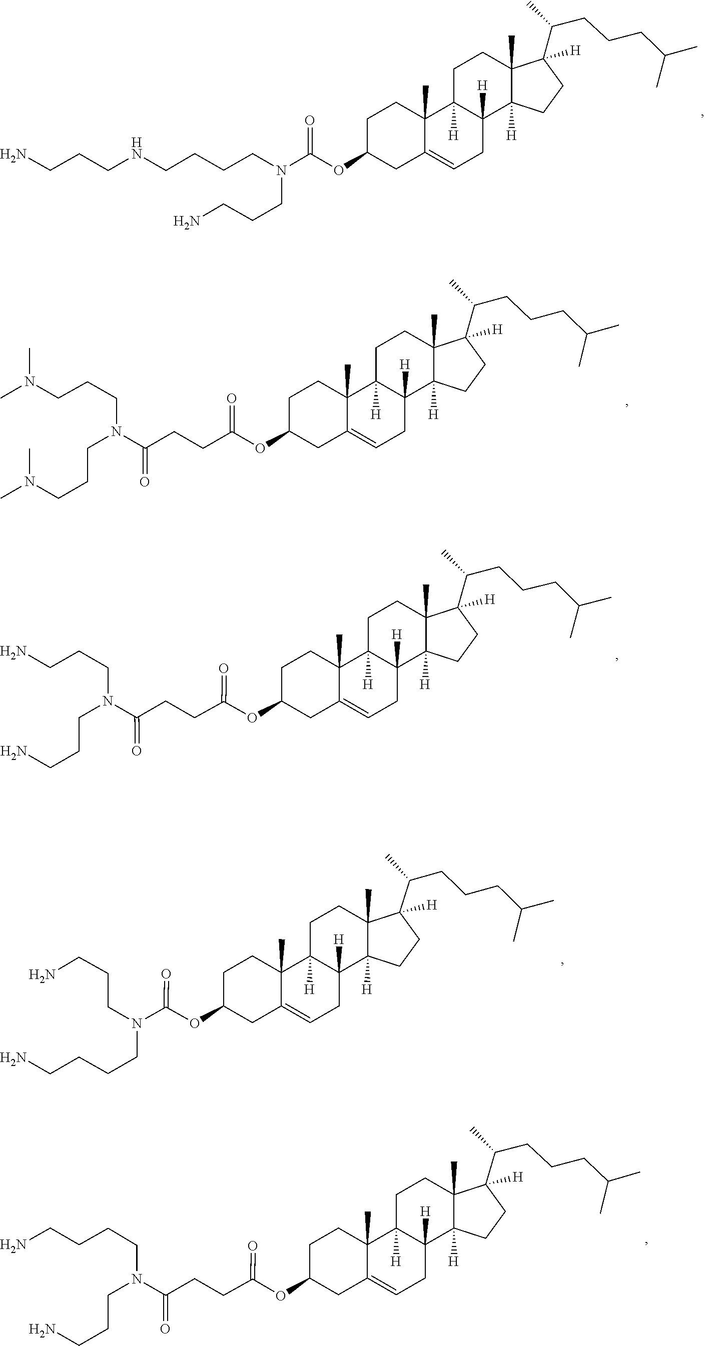

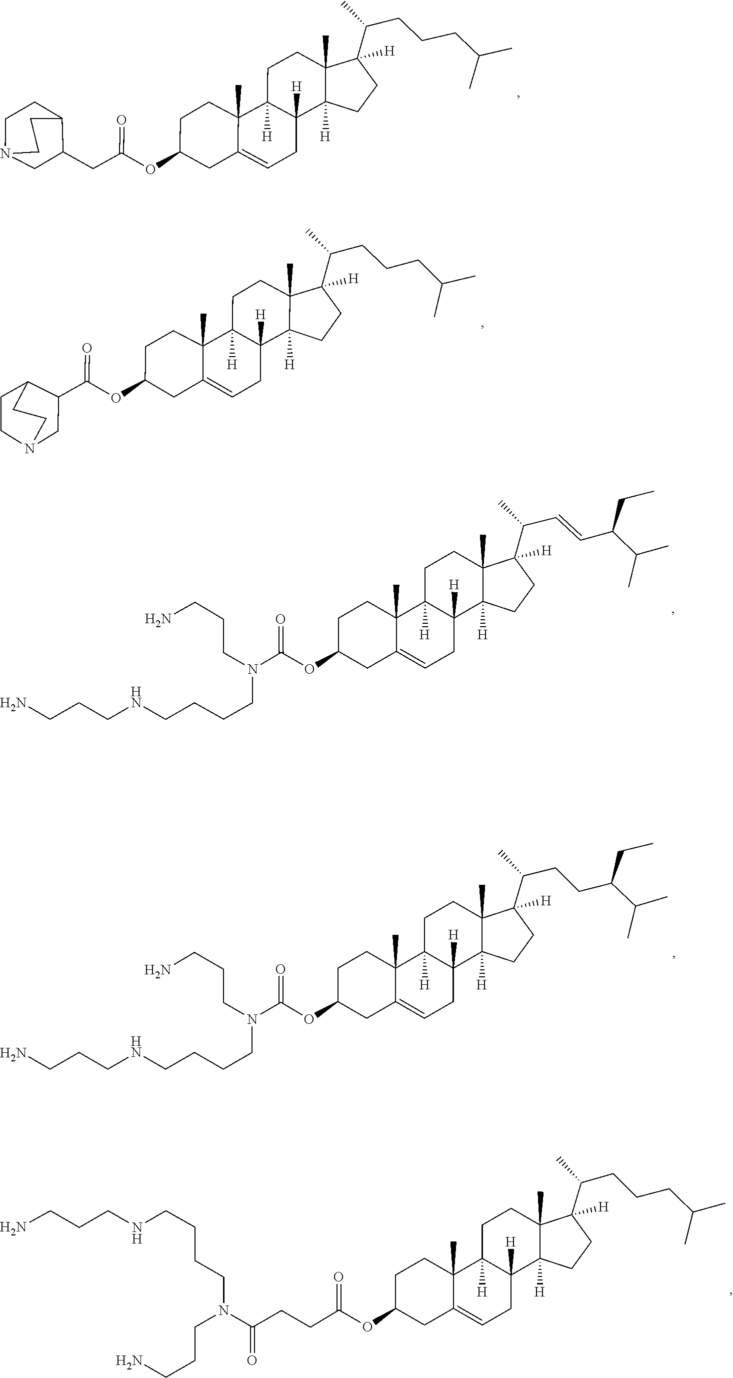

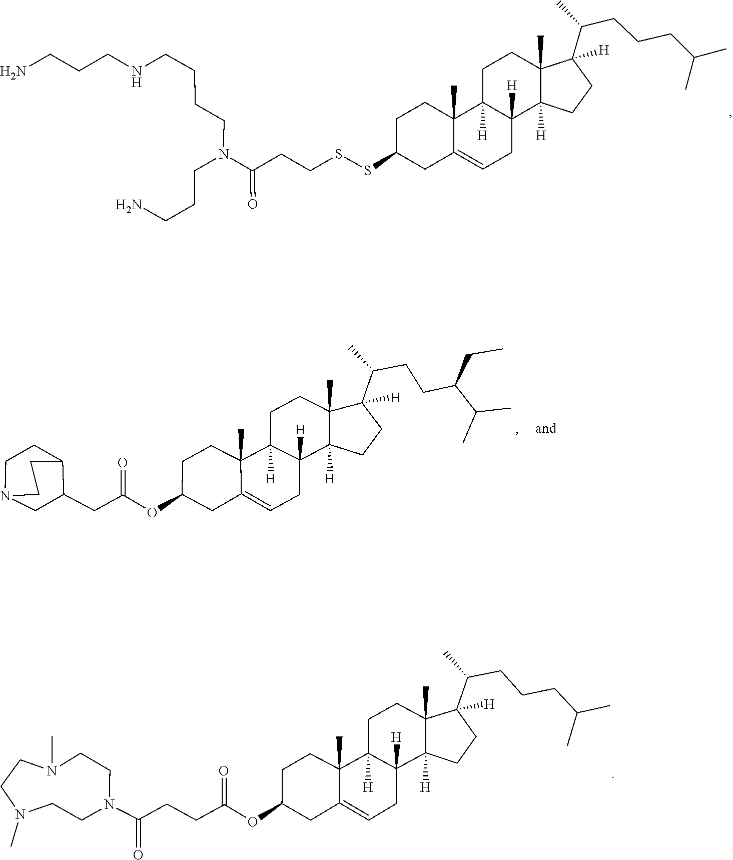

In some embodiments, Z is N. In some embodiments, Z is CH.

In some embodiments, R¹ is C₁₋₁₄ alkyl. In some embodiments, R¹ is C₃₋₁₂ alkyl. In some embodiments, R¹ is C₆₋₁₂ alkyl. In some embodiments, R¹ is C₈₋₁₀ alkyl. In some embodiments, R¹ is C₈ alkyl. In some embodiments, R¹ is C₁₀ alkyl.

In some embodiments, R¹ is C₁₋₁₄ hydroxyalkyl. In some embodiments, R¹ is C₃₋₁₂ hydroxyalkyl. In some embodiments, R¹ is C₆₋₁₂ hydroxyalkyl. In some embodiments, R¹ is C₈₋₁₀ hydroxyalkyl. In some embodiments, R¹ is C₈ hydroxyalkyl. In some embodiments, R¹ is C₁₀ hydroxyalkyl.

In some embodiments, R¹ is C₁₋₁₄ alkenyl. In some embodiments, R¹ is C₃₋₁₂ alkenyl. In some embodiments, R¹ is C₆₋₁₂ alkenyl. In some embodiments, R¹ is C₈₋₁₀ alkenyl. In some embodiments, R¹ is C₈ alkenyl. In some embodiments, R¹ is C₁₀ alkenyl.

In some embodiments, R¹ is

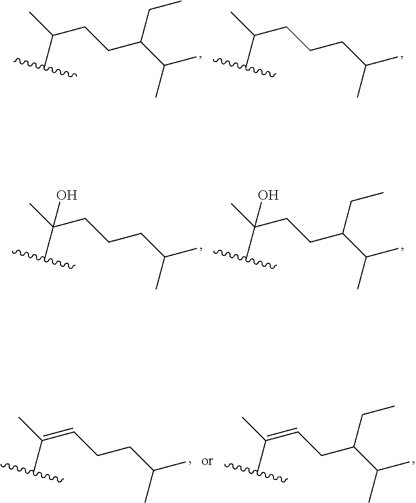

In some embodiments R¹ is

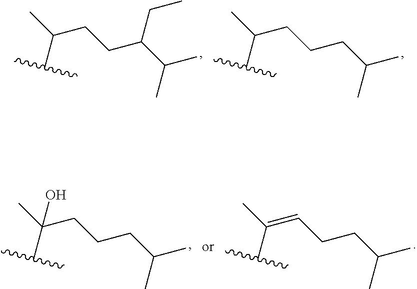

In some embodiments, R¹ is

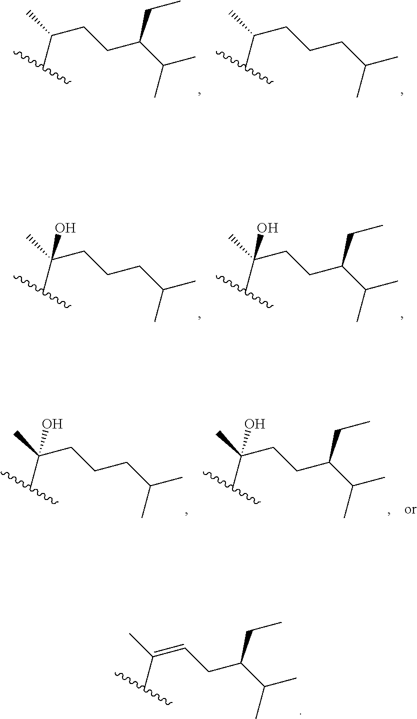

In some embodiments, R¹ is

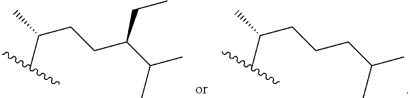

In some embodiments, when j is 1, then 1 is 0.

In some embodiments, when j is 0, then 1 is 1.

In some embodiments, when one of j and 1 is 1, then the other is 0.

In some embodiments, j is 0. In some embodiments, j is 1.

In some embodiments, k is 0, 1, 2, 3, or 4. In some embodiments, k is 0, 2, 3, or 4. In some embodiments, k is 0. In some embodiments, k is 1. In some embodiments, k is 2. In some embodiments, k is 3. In some embodiments, k is 4. In some embodiments, k is 5. In some embodiments, k is 6.

In some embodiments, 1 is 0. In some embodiments, 1 is 1.

In some embodiments, m is 0, 1, 2, or 4. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is 0. In some embodiments, n is 1.

In some embodiments, j is 0, k is 0, 1 is 1, m is 1, and n is 1. In some embodiments, j is 0, k is 0, 1 is 1, m is 2, and n is 1. In some embodiments, j is 0, k is 0, 1 is 1, m is 4, and n is 1. In some embodiments, j is 1, k is 0, 1 is 0, m is 0, and n is 0. In some embodiments, j is 1, k is 1, 1 is 0, m is 0, and n is 0. In some embodiments, j is 1, k is 1, 1 is 0, m is 0, and n is 1. In some embodiments, j is 1, k is 1, 1 is 0, m is 2, and n is 0.

In some embodiments, j is 1, k is 1, 1 is 1, m is 1, and n is 1. In some embodiments, j is 1, k is 2, 1 is 0, m is 0, and n is 0. In some embodiments, j is 1, k is 2, 1 is 0, m is 0, and n is 1. In some embodiments, j is 1, k is 3, 1 is 0, m is 0, and n is 1. In some embodiments, j is 1, k is 4, 1 is 0, m is 0, and n is 1.

In some embodiments, k is 1 and both R⁴ and R⁵ are H. In some embodiments, k is 1 and one of R⁴ and R⁵ is C₁₋₄ alkyl and the other of R⁴ and R⁵ is H. In some embodiments, k is 1 and one of R⁴ and R⁵ is methyl and the other of R⁴ and R⁵ is H. In some embodiments, k is 2 and each R⁴ and R⁵ is H. In some embodiments, k is 2 and one R⁴ is C₁₋₄ alkyl and the remaining R⁴ and R⁵ substituents are H. In some embodiments, k is 2 and one R⁴ is methyl and the remaining R⁴ and R⁵ substituents are H. In some embodiments, k is 3 and each R⁴ and R⁵ is H. In some embodiments, k is 4 and each R⁴ and R⁵ is H.

In some embodiments, m is 1 and both R⁶ and R⁷ are H. In some embodiments, m is 2 and each R⁶ and R⁷ is H. In some embodiments, m is 4 and each R⁶ and R⁷ is H. In some embodiments, m is 2, one R⁶ with R² and R³ form, together with the atoms to which they are attached and any intervening atoms, a 7-18 membered bridged heterocycloalkyl group and the other R⁶ is H, and both R⁷ are H.

In some embodiments, j is 0, k is 0, 1 is 1, m is 1, both R⁶ and R⁷ are H, and n is 1. In some embodiments, j is 0, k is 0, 1 is 1, m is 2, each R⁶ and R⁷ is H, and n is 1. In some embodiments, j is 0, k is 0, 1 is 1, m is 4, each R⁶ and R⁷ is H, and n is 1. In some embodiments, j is 1, k is 1, each R⁴ and R⁵ is H, 1 is 0, m is 0, and n is 0. In some embodiments, j is 1, k is 1, one of R⁴ and R⁵ is C₁₋₄ alkyl and the other of R⁴ and R⁵ is H, 1 is 0, m is 0, and n is 0. In some embodiments, j is 1, k is 1, each R⁴ and R⁵ is H, 1 is 0, m is 0, and n is 1. In some embodiments, j is 1, k is 1, one of R⁴ and R⁵ is C₁₋₄ alkyl and the other of R⁴ and R⁵ is H, 1 is 0, m is 0, and n is 1. In some embodiments, j is 1, k is 2, each R⁴ and R⁵ is H, 1 is 0, m is 0, and n is 0. In some embodiments, j is 1, k is 2, one R⁴ is C₁₋₄ alkyl and the remaining R⁴ and R⁵ substituents are H, 1 is 0, m is 0, and n is 0. In some embodiments, j is 1, k is 2, each R⁴ and R⁵ is H, 1 is 0, m is 0, and n is 1. In some embodiments, j is 1, k is 3, each R⁴ and R⁵ is H, 1 is 0, m is 0, and n is 1. In some embodiments, j is 1, k is 4, each R⁴ and R⁵ is H, 1 is 0, m is 0, and n is 1. In some embodiments, j is 1, k is 1, each R⁴ and R⁵ is H, 1 is 1, m is 1, both R⁶ and R⁷ are H, and n is 1. In some embodiments, j is 1, k is 1, each R⁴ and R⁵ is H, 1 is 0, m is 2, one of R⁶ with R² and R³ form, together with the atoms to which they are attached and any intervening atoms, a 7-18 membered bridged heterocycloalkyl group and the other R⁶ is H, both R⁷ are H, and n is 0.

In some embodiments, j is 1, k is 1, one of R⁴ and R⁵ is methyl and the other of R⁴ and R⁵ is H, 1 is 0, m is 0, and n is 0. In some embodiments, j is 1, k is 1, one of R⁴ and R⁵ is methyl and the other of R⁴ and R⁵ is H, 1 is 0, m is 0, and n is 1. In some embodiments, j is 1, k is 2, one of R⁴ is methyl and the remaining R⁴ and R⁵ substituents are H, 1 is 0, m is 0, and n is 0.

In some embodiments, R² and R³ are each independently selected from C₂₋₁₀ alkyl, wherein the C₂₋₁₀ alkyl is substituted by 1, 2, 3, 4, or 5 substituents independently selected from —NR⁸R⁹, OH, and halo, wherein at least one substituent is —NR⁸R⁹.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, OH, and halo, wherein at least one substituent is —NR⁸R⁹; and
(ii) 1, 2, 3, or 4 non-terminal carbons of the C₂₋₁₀ alkyl are optionally replaced with O.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, OH, and halo, wherein at least one substituent is —NR⁸R⁹; and
(iii) 1, 2, 3, or 4 non-terminal carbons of the C₂₋₁₀ alkyl are optionally replaced with NR¹⁰.

In some embodiments, R² and R³ are each independently selected from C₂₋₁₀ alkyl, wherein:
(i) the C₂₋₁₀ alkyl is substituted by 1, 2, 3, 4, or 5 substituents independently selected from —NR⁸R⁹, OH, and halo, wherein at least one substituent is —NR⁸R⁹; and
(iv) 1, 2, 3, or 4 non-terminal carbons of the C₂₋₁₀ alkyl are optionally replaced with C(═O).

In some embodiments, R² and R³ are each independently selected from C₂₋₁₀ alkyl, wherein:
(i) the C₂₋₂₀ alkyl is substituted by 1, 2, 3, 4, or 5 substituents independently selected from —NR⁸R⁹, OH, and halo, wherein at least one substituent is —NR⁸R⁹; and
(v) 1, 2, 3, or 4 non-terminal carbons of the C₂₋₂₀ alkyl are optionally replaced with CR^{a}R^{b} wherein R^{a} and R^{b} together with the C atom to which they are attached form a C₃₋₆ cycloalkyl group.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₂₋₁₀ alkyl are optionally replaced with O;
(iii) 1 or 2 non-terminal carbons of the C₂₋₁₀ alkyl are optionally replaced with NR¹⁰.
(iv) 1 or 2 non-terminal carbons of the C₂₋₁₀ alkyl are optionally replaced with C(═O); and
(v) 1 or 2 non-terminal carbons of the C₂₋₁₀ alkyl are optionally replaced with CR^{a}R^{b} wherein R^{a} and R^{b} together with the C atom to which they are attached form a C₃₋₆ cycloalkyl group.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹; and
(ii) 1 or 2 non-terminal carbons of the C₂₋₁₀ alkyl are optionally replaced with O.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹; and
(iii) 1 or 2 non-terminal carbons of the C₂₋₁₀ alkyl are optionally replaced with NR¹⁰.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹; and
(iv) 1 or 2 non-terminal carbons of the C₂₋₁₀ alkyl are optionally replaced with C(═O).

In some embodiments, R² and R³ are each independently selected from C₂₋₁₀ alkyl, wherein:
(i) the C₂₋₂₀ alkyl is substituted by 1, 2, 3, or 4 substituents independently selected from —NR⁸R⁹, OH, and halo, wherein at least one substituent is —NR⁸R⁹; and
(v) 1 or 2 non-terminal carbons of the C₂₋₁₀ alkyl are optionally replaced with CR^{a}R^{b} wherein R^{a} and R^{b} together with the C atom to which they are attached form a C₃₋₆ cycloalkyl group.

In some embodiments, R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₂₋₁₀ alkyl are optionally replaced with O; and
(iii) 1 or 2 non-terminal carbons of the C₂₋₁₀ alkyl are optionally replaced with NR¹⁰.

In some embodiments, R² and R³ are each independently selected from C₄₋₁₀ alkyl, wherein:
(i) the C₄₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₄₋₁₀ alkyl are optionally replaced with O;
(iii) 1 or 2 non-terminal carbons of the C₄₋₁₀ alkyl are optionally replaced with NR¹⁰;
(iv) 1 or 2 non-terminal carbons of the C₄₋₁₀ alkyl are optionally replaced with C(═O); and
(v) 1 or 2 non-terminal carbons of the C₄₋₁₀ alkyl are optionally replaced with CR^{a}R^{b} wherein R^{a} and R^{b} together with the C atom to which they are attached form a C₃₋₆ cycloalkyl group.

In some embodiments, R² and R³ are each independently selected from C₄₋₁₀ alkyl, wherein:
(i) the C₄₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₄₋₁₀ alkyl are optionally replaced with O; and
(iii) 1 or 2 non-terminal carbons of the C₄₋₁₀ alkyl are optionally replaced with NR¹⁰.

In some embodiments, one of R² and R³ is C₂₋₅ alkyl, wherein:

the C₂₋₅ alkyl is substituted by 1, 2, 3, 4, or 5 substituents independently selected from —NR⁸R⁹, OH, and halo, wherein at least one substituent is —NR⁸R⁹;
and wherein the other of R² and R³ is C₇₋₁₀ alkyl, wherein:
(i) the C₇₋₁₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₇₋₁₀ alkyl are optionally replaced with O;
(iii) 1, 2, 3, or 4 non-terminal carbons of the C₇₋₁₀ alkyl are optionally replaced with NR¹⁰;
(iv) 1, 2, 3, or 4 non-terminal carbons of the C₇₋₁₀ alkyl are optionally replaced with C(═O); and
(v) 1, 2, 3, or 4 non-terminal carbons of the C₇₋₁₀ alkyl are optionally replaced with CR^{a}R^{b} wherein R^{a} and R^{b} together with the C atom to which they are attached form a C₃₋₆ cycloalkyl group.

In some embodiments, one of R² and R³ is C₂₋₅ alkyl, wherein:
(i) the C₂₋₅ alkyl is substituted by 1, 2, 3, 4, or 5 substituents independently selected from —NR⁸R⁹, OH, and halo, wherein at least one substituent is —NR⁸R⁹; and wherein the other of R² and R³ is C₇₋₁₀ alkyl, wherein:
   (i) the C₇₋₁₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₇₋₁₀ alkyl are optionally replaced with O; and
(iii) 1, 2, 3, or 4 non-terminal carbons of the C₇₋₁₀ alkyl are optionally replaced with NR¹⁰.

In some embodiments, one of R² and R³ is C₂₋₂₀ alkyl substituted by 1 —NR⁸R⁹. In some embodiments, one of R² and R³ is C₂₋₂₀ alkyl substituted by 1 —NR⁸R⁹ and 1 non-terminal carbon of the C₂₋₂₀ alkyl is replaced with NR¹⁰. In some embodiments, one of R² and R³ is C₂₋₂₀ alkyl substituted by 1 —NR⁸R⁹ and 1 non-terminal carbon of the C₂₋₂₀ alkyl is replaced with O. In some embodiments, one of R² and R³ is C₂₋₂₀ alkyl substituted by 1 —NR⁸R⁹ and 2 halo and 1 non-terminal carbon of the C₂₋₂₀ alkyl is replaced with NR¹⁰. In some embodiments, one of R² and R³ is C₂₋₂₀ alkyl substituted by 1 —NR⁸R⁹ and 2 —F and 1 non-terminal carbon of the C₂₋₂₀ alkyl is replaced with NR¹⁰. In some embodiments, one of R² and R³ is C₂₋₂₀ alkyl substituted by 1 —NR⁸R⁹ and 2 halo. In some embodiments, one of R² and R³ is C₂₋₂₀ alkyl substituted by 1 —NR⁸R⁹ and 2 —F. In some embodiments, one of R² and R³ is C₂₋₂₀ alkyl substituted by 1 —NR⁸R⁹ and 1 halo, wherein 1 non-terminal carbon of the C₂₋₂₀ alkyl is replaced with NR¹⁰. In some embodiments, one of R² and R³ is C₂₋₂₀ alkyl substituted by 1 —NR⁸R⁹ and 1 —F, wherein 1 non-terminal carbon of the C₂₋₂₀ alkyl is replaced with NR¹⁰. In some embodiments, one of R² and R³ is C₂₋₂₀ alkyl substituted by 1 —NR⁸R⁹ and 1 halo. In some embodiments, one of R² and R³ is C₂₋₂₀ alkyl substituted by 1 —NR⁸R⁹ and 1 —F. In some embodiments, one of R² and R³ is C₂₋₂₀ alkyl substituted by 1 —NR⁸R⁹ and 1 OH.

In some embodiments, one of R² and R³ is C₂₋₂₀ alkyl substituted by 1 —NR⁸R⁹ and 1 non-terminal carbon of the C₂₋₂₀ alkyl is replaced with C(═O). In some embodiments, one of R² and R³ is C₂₋₂₀ alkyl substituted by 2 —NR⁸R⁹ and 1 non-terminal carbon of the C₂₋₂₀ alkyl is replaced with C(═O). In some embodiments, one of R² and R³ is C₂₋₂₀ alkyl substituted by 1 —NR⁸R⁹, 1 non-terminal carbon of the C₂₋₂₀ alkyl is replaced with NR¹⁰, and 1 non-terminal carbon of the C₂₋₂₀ alkyl is replaced with CR^{a}R^{b} wherein R^{a} and R^{b} together with the C atom to which they are attached form a C₃₋₆ cycloalkyl group. In some embodiments, one of R² and R³ is C₂₋₂₀ alkyl substituted by 1 —NR⁸R⁹ and 1 non-terminal carbon of the C₂₋₂₀ alkyl is replaced with CR^{a}R^{b} wherein R^{a} and R^{b} together with the C atom to which they are attached form a C₃₋₆ cycloalkyl group.

In some embodiments, one of R² and R³ is selected from:
C₂₋₂₀ alkyl substituted by 1 —NR⁸R⁹,
C₂₋₂₀ alkyl substituted by 1 —NR⁸R⁹ wherein 1 non-terminal carbon of the C₂₋₂₀ alkyl is replaced with NR¹⁰,
C₂₋₂₀ alkyl substituted by 1 —NR⁸R⁹ wherein 1 non-terminal carbon of the C₂₋₂₀ alkyl is replaced with O,
C₂₋₂₀ alkyl substituted by 1 —NR⁸R⁹ and 2 halo wherein 1 non-terminal carbon of the C₂₋₂₀ alkyl is replaced with NR¹⁰,
C₂₋₂₀ alkyl substituted by 1 —NR⁸R⁹ and 1 halo wherein 1 non-terminal carbon of the C₂₋₂₀ alkyl is replaced with NR¹⁰,
C₂₋₂₀ alkyl substituted by 1 —NR⁸R⁹ wherein 1 non-terminal carbon of the C₂₋₂₀ alkyl is replaced with C(═O),
C₂₋₂₀ alkyl substituted by 2 —NR⁸R⁹ wherein 1 non-terminal carbon of the C₂₋₂₀ alkyl is replaced with C(═O), and
C₂₋₂₀ alkyl substituted by 1 —NR⁸R⁹ wherein 1 non-terminal carbon of the C₂₋₂₀ alkyl is replaced with NR¹⁰ and 1 non-terminal carbon of the C₂₋₂₀ alkyl is replaced with CR^{a}R^{b} wherein R^{a} and R^{b} together with the C atom to which they are attached form a C₃₋₆ cycloalkyl group, and
   the other of R² and R³ is selected from:
C₂₋₂₀ alkyl substituted by 1 —NR⁸R⁹,
C₂₋₂₀ alkyl substituted by 1 —NR⁸R⁹ wherein 1 non-terminal carbon of the C₂₋₂₀ alkyl is replaced with NR¹⁰,
C₂₋₂₀ alkyl substituted by 1 —NR⁸R⁹ and 2 halo,
C₂₋₂₀ alkyl substituted by 1 —NR⁸R⁹ and 1 halo,
C₂₋₂₀ alkyl substituted by 1 —NR⁸R⁹ and 1 OH, and
C₂₋₂₀ alkyl substituted by 1—NR⁸R⁹ wherein 1 non-terminal carbon of the C₂₋₂₀ alkyl is replaced with CR^{a}R^{b} wherein R^{a} and R^{b} together with the C atom to which they are attached form a C₃₋₆ cycloalkyl group.

In some embodiments, one of R² and R³ is selected from C₂₋₂₀ alkyl substituted by 1 —NR⁸R⁹, C₂₋₂₀ alkyl substituted by 1 —NR⁸R⁹ wherein 1 non-terminal carbon of the C₂₋₂₀ alkyl is replaced with NR¹⁰, C₂₋₂₀ alkyl substituted by 1 —NR⁸R⁹ wherein 1 non-terminal carbon of the C₂₋₂₀ alkyl is replaced with O, C₂₋₂₀ alkyl substituted by 1 —NR⁸R⁹ and 2 halo wherein 1 non-terminal carbon of the C₂₋₂₀ alkyl is replaced with NR¹⁰, and C₂₋₂₀ alkyl substituted by 1 —NR⁸R⁹ and 1 halo wherein 1 non-terminal carbon of the C₂₋₂₀ alkyl is replaced with NR¹⁰, and the other of R² and R³ is selected from C₂₋₂₀ alkyl substituted by 1 —NR⁸R⁹, C₂₋₂₀ alkyl substituted by 1 —NR⁸R⁹ wherein 1 non-terminal carbon of the C₂₋₂₀ alkyl is replaced with NR¹⁰, C₂₋₂₀ alkyl substituted by 1 —NR⁸R⁹ and 2 halo, C₂₋₂₀ alkyl substituted by 1 —NR⁸R⁹ and 1 halo, and C₂₋₂₀ alkyl substituted by 1 —NR⁸R⁹ and 1 OH.

In some embodiments, one of R² and R³ is C₂₋₁₀ alkyl substituted by 1 —NR⁸R⁹. In some embodiments, one of R² and R³ is C₂₋₁₀ alkyl substituted by 1 —NR⁸R⁹ and 1 non-terminal carbon of the C₂₋₁₀ alkyl is replaced with NR¹⁰. In some embodiments, one of R² and R³ is C₂₋₁₀ alkyl substituted by 1 —NR⁸R⁹ and 1 non-terminal carbon of the C₂₋₁₀ alkyl is replaced with O. In some embodiments, one of R² and R³ is C₂₋₁₀ alkyl substituted by 1 —NR⁸R⁹ and 2 halo and 1 non-terminal carbon of the C₂₋₁₀ alkyl is replaced with NR¹⁰. In some embodiments, one of R² and R³ is C₂₋₁₀ alkyl substituted by 1 —NR⁸R⁹ and 2 —F and 1 non-terminal carbon of the C₂₋₁₀ alkyl is replaced with NR¹⁰. In some embodiments, one of R² and R³ is C₂₋₁₀ alkyl substituted by 1 —NR⁸R⁹ and 2 halo. In some embodiments, one of R² and R³ is C₂₋₁₀ alkyl substituted by 1 —NR⁸R⁹ and 2 —F. In some embodiments, one of R² and R³ is C₂₋₁₀ alkyl substituted by 1 —NR⁸R⁹ and 1 halo wherein 1 non-terminal carbon of the C₂₋₁₀ alkyl is replaced with NR¹⁰. In some embodiments, one of R² and R³ is C₂₋₁₀ alkyl substituted by 1 —NR⁸R⁹ and 1 —F wherein 1 non-terminal carbon of the C₂₋₁₀ alkyl is replaced with NR¹⁰. In some embodiments, one of R² and R³ is C₂₋₁₀ alkyl substituted by 1 —NR⁸R⁹ and 1 halo. In some embodiments, one of R² and R³ is C₂₋₁₀ alkyl substituted by 1 —NR⁸R⁹ and 1 —F. In some embodiments, one of R² and R³ is C₂₋₁₀ alkyl substituted by 1 —NR⁸R⁹ and 1 OH.

In some embodiments, one of R² and R³ is C₂₋₁₀ alkyl substituted by 1 —NR⁸R⁹ and 1 non-terminal carbon of the C₂₋₁₀ alkyl is replaced with C(═O). In some embodiments, one of R² and R³ is C₂₋₁₀ alkyl substituted by 2 —NR⁸R⁹ and 1 non-terminal carbon of the C₂₋₁₀ alkyl is replaced with C(═O). In some embodiments, one of R² and R³ is C₂₋₁₀ alkyl substituted by 1 —NR⁸R⁹, 1 non-terminal carbon of the C₂₋₁₀ alkyl is replaced with NR¹⁰ and 1 non-terminal carbon of the C₂₋₁₀ alkyl is replaced with CR^{a}R^{b} wherein R^{a} and R^{b} together with the C atom to which they are attached form a C₃₋₆ cycloalkyl group. In some embodiments, one of R² and R³ is C₂₋₁₀ alkyl substituted by 1—NR⁸R⁹ and 1 non-terminal carbon of the C₂₋₁₀ alkyl is replaced with CR^{a}R^{b} wherein R^{a} and R^{b} together with the C atom to which they are attached form a C₃₋₆ cycloalkyl group.

In some embodiments, one of R² and R³ is selected from:
C₂₋₁₀ alkyl substituted by 1 —NR⁸R⁹,
C₂₋₁₀ alkyl substituted by 1 —NR⁸R⁹ wherein 1 non-terminal carbon of the C₂₋₁₀ alkyl is replaced with NR¹⁰,
C₂₋₁₀ alkyl substituted by 1 —NR⁸R⁹ wherein 1 non-terminal carbon of the C₂₋₁₀ alkyl is replaced with O,
C₂₋₁₀ alkyl substituted by 1 —NR⁸R⁹ and 2 halo wherein 1 non-terminal carbon of the C₂₋₁₀ alkyl is replaced with NR¹⁰,
C₂₋₁₀ alkyl substituted by 1 —NR⁸R⁹ and 1 halo wherein 1 non-terminal carbon of the C₂₋₁₀ alkyl is replaced with NR¹⁰,
C₂₋₁₀ alkyl substituted by 1 —NR⁸R⁹ wherein 1 non-terminal carbon of the C₂₋₁₀ alkyl is replaced with C(═O),
C₂₋₁₀ alkyl substituted by 2 —NR⁸R⁹ wherein 1 non-terminal carbon of the C₂₋₁₀ alkyl is replaced with C(═O), and
C₂₋₁₀ alkyl substituted by 1 —NR⁸R⁹ wherein 1 non-terminal carbon of the C₂₋₁₀ alkyl is replaced with NR¹⁰ and 1 non-terminal carbon of the C₂₋₁₀ alkyl is replaced with CR^{a}R^{b} wherein R^{a} and R^{b} together with the C atom to which they are attached form a C₃₋₆ cycloalkyl group, and
   the other of R² and R³ is selected from:
C₂₋₁₀ alkyl substituted by 1 —NR⁸R⁹,
C₂₋₁₀ alkyl substituted by 1 —NR⁸R⁹ wherein 1 non-terminal carbon of the C₂₋₁₀ alkyl is replaced with NR¹⁰,
C₂₋₁₀ alkyl substituted by 1 —NR⁸R⁹ and 2 halo,
C₂₋₁₀ alkyl substituted by 1 —NR⁸R⁹ and 1 halo,
C₂₋₁₀ alkyl substituted by 1 —NR⁸R⁹ and 1 OH, and
C₂₋₁₀ alkyl substituted by 1—NR⁸R⁹ wherein 1 non-terminal carbon of the C₂₋₁₀ alkyl is replaced with CR^{a}R^{b} wherein R^{a} and R^{b} together with the C atom to which they are attached form a C₃₋₆ cycloalkyl group.

In some embodiments, one of R² and R³ is selected from C₂₋₁₀ alkyl substituted by 1 —NR⁸R⁹, C₂₋₁₀ alkyl substituted by 1 —NR⁸R⁹ wherein 1 non-terminal carbon of the C₂₋₁₀ alkyl is replaced with NR¹⁰, C₂₋₁₀ alkyl substituted by 1 —NR⁸R⁹ wherein 1 non-terminal carbon of the C₂₋₁₀ alkyl is replaced with O, C₂₋₁₀ alkyl substituted by 1 —NR⁸R⁹ and 2 halo wherein 1 non-terminal carbon of the C₂₋₁₀ alkyl is replaced with NR¹⁰, and C₂₋₁₀ alkyl substituted by 1 —NR⁸R⁹ and 1 halo wherein 1 non-terminal carbon of the C₂₋₁₀ alkyl is replaced with NR¹⁰,and the other of R² and R³ is selected from C₂₋₁₀ alkyl substituted by 1 —NR⁸R⁹, C₂₋₁₀ alkyl substituted by 1 —NR⁸R⁹ wherein 1 non-terminal carbon of the C₂₋₁₀ alkyl is replaced with NR¹⁰, C₂₋₁₀ alkyl substituted by 1 —NR⁸R⁹ and 2 halo, C₂₋₁₀ alkyl substituted by 1 —NR⁸R⁹ and 1 halo, and C₂₋₁₀ alkyl substituted by 1 —NR⁸R⁹ and 1 OH.

In some embodiments, one of R² and R³ is selected from:
C₅₋₁₀ alkyl substituted by 1 —NR⁸R⁹,
C₅₋₁₀ alkyl substituted by 1 —NR⁸R⁹ wherein 1 non-terminal carbon of the C₈₋₁₀ alkyl is replaced with NR¹⁰,
C₅₋₁₀ alkyl substituted by 1 —NR⁸R⁹ wherein 1 non-terminal carbon of the C₈₋₁₀ alkyl is replaced with O,
C₅₋₁₀ alkyl substituted by 1 —NR⁸R⁹ and 2 halo wherein 1 non-terminal carbon of the C₅₋₁₀ alkyl is replaced with NR¹⁰,
C₅₋₁₀ alkyl substituted by 1 —NR⁸R⁹ and 1 halo wherein 1 non-terminal carbon of the C₅₋₁₀ alkyl is replaced with NR¹⁰, and
C₅₋₁₀ alkyl substituted by 1 —NR⁸R⁹ wherein 1 non-terminal carbon of the C₂₋₁₀ alkyl is replaced with NR¹⁰ and 1 non-terminal carbon of the C₂₋₁₀ alkyl is replaced with CR^{a}R^{b} wherein R^{a} and R^{b} together with the C atom to which they are attached form a C₃₋₆ cycloalkyl group,
   and the other of R² and R³ is selected from:
C₃₋₆ alkyl substituted by 1 —NR⁸R⁹,
C₃₋₆ alkyl substituted by 1 —NR⁸R⁹ wherein 1 non-terminal carbon of the C₃₋₆ alkyl is replaced with NR¹⁰,
C₃₋₆ alkyl substituted by 1 —NR⁸R⁹ and 2 halo,
C₃₋₆ alkyl substituted by 1 —NR⁸R⁹ and 1 halo,
C₃₋₆ alkyl substituted by 1 —NR⁸R⁹ and 1 OH,
C₃₋₆ alkyl substituted by 1 —NR⁸R⁹ wherein 1 non-terminal carbon of the C₂₋₁₀ alkyl is replaced with C(═O),
C₃₋₆ alkyl substituted by 2 —NR⁸R⁹ wherein 1 non-terminal carbon of the C₂₋₁₀ alkyl is replaced with C(═O), and
C₃₋₆ alkyl substituted by 1—NR⁸R⁹ wherein 1 non-terminal carbon of the C₂₋₁₀ alkyl is replaced with CR^{a}R^{b} wherein R^{a} and R^{b} together with the C atom to which they are attached form a C₃₋₆ cycloalkyl group.

In some embodiments, one of R² and R³ is selected from C₅₋₁₀ alkyl substituted by 1 —NR⁸R⁹, C₅₋₁₀ alkyl substituted by 1 —NR⁸R⁹ wherein 1 non-terminal carbon of the C₈₋₁₀ alkyl is replaced with NR¹⁰, C₅₋₁₀ alkyl substituted by 1 —NR⁸R⁹ wherein 1 non-terminal carbon of the C₅₋₁₀ alkyl is replaced with O, C₅₋₁₀ alkyl substituted by 1 —NR⁸R⁹ and 2 halo wherein 1 non-terminal carbon of the C₅₋₁₀ alkyl is replaced with NR¹⁰, and C₅₋₁₀ alkyl substituted by 1 —NR⁸R⁹ and 1 halo wherein 1 non-terminal carbon of the C₅₋₁₀ alkyl is replaced with NR¹⁰, and the other of R² and R³ is selected from C₃₋₆ alkyl substituted by 1 —NR⁸R⁹, C₃₋₆ alkyl substituted by 1 —NR⁸R⁹ wherein 1 non-terminal carbon of the C₃₋₆ alkyl is replaced with NR¹⁰, C₃₋₆ alkyl substituted by 1 —NR⁸R⁹ and 2 halo, C₃₋₆ alkyl substituted by 1 —NR⁸R⁹ and 1 halo, and C₃₋₆ alkyl substituted by 1 —NR⁸R⁹ and 1 OH.

In some embodiments, one of R² and R³ is C₃ alkyl which is substituted by at least one —NR⁸R⁹ group and is further optionally substituted by one or two groups selected from OH and halo.

In some embodiments, one of R² and R³ is selected from

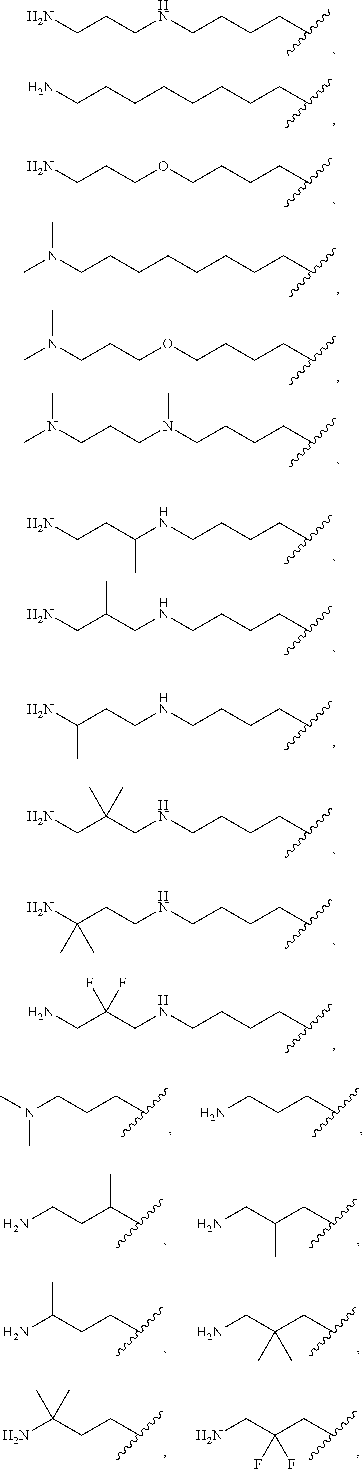

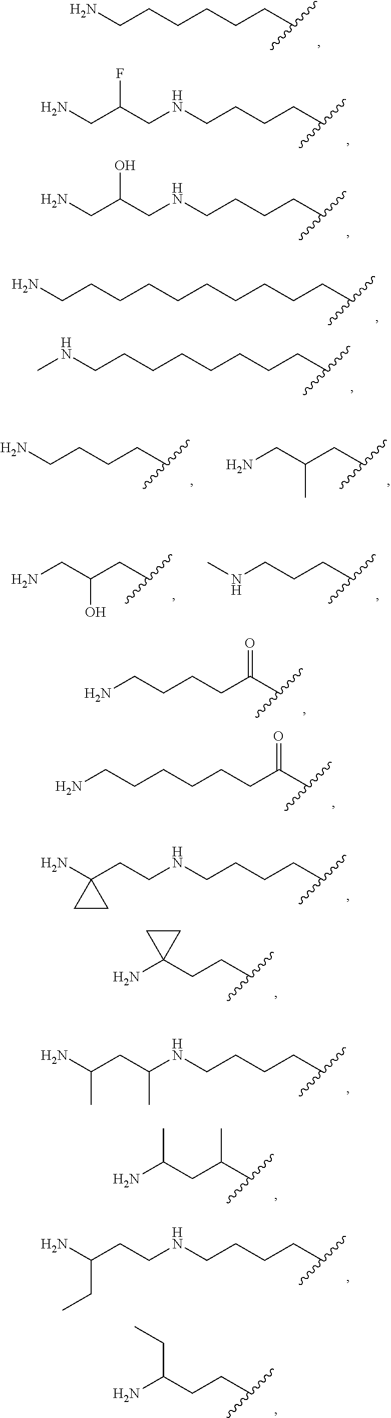

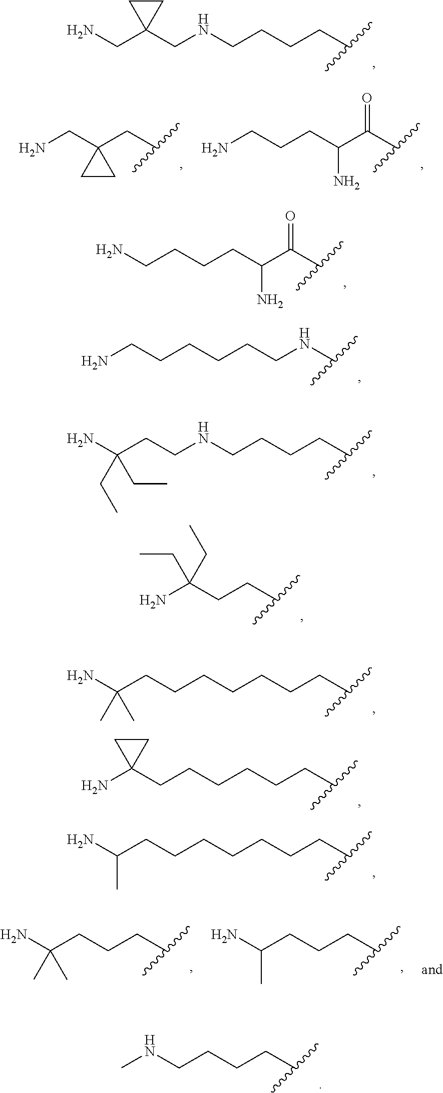

In some embodiments, one of R² and R³ is selected from

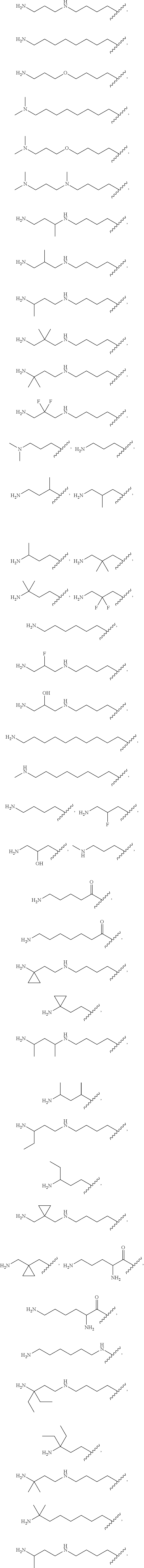

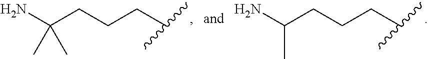

In some embodiments, one of R² and R³ is selected from

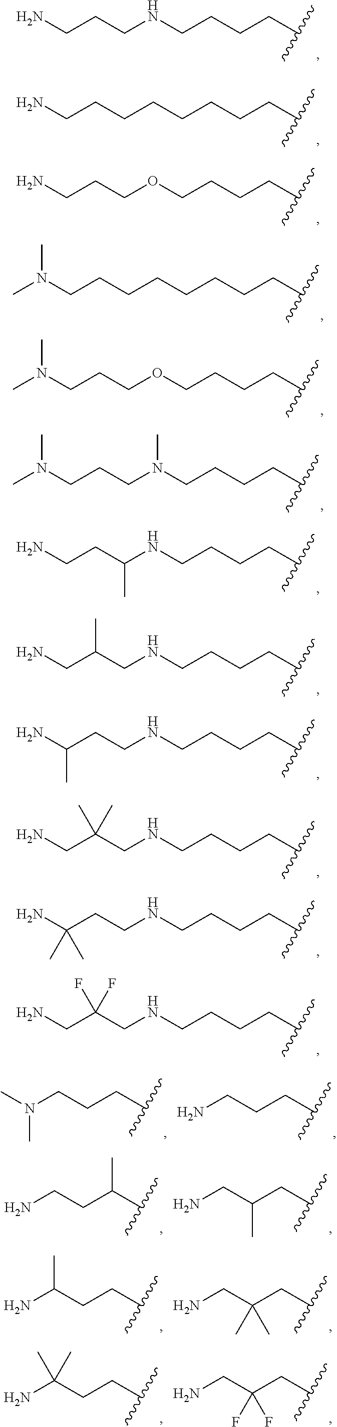

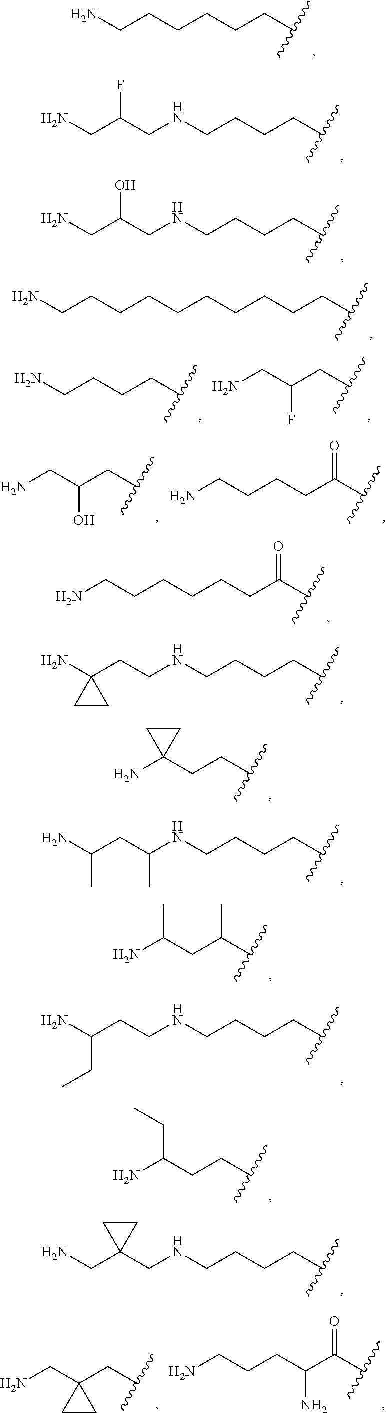

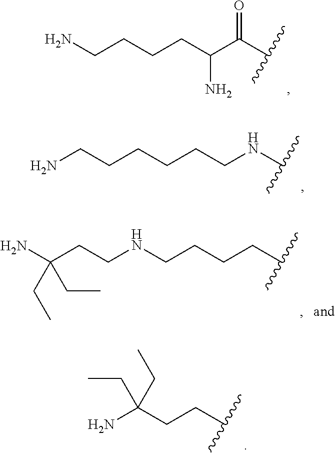

In some embodiments, one of R² and R³ is selected from

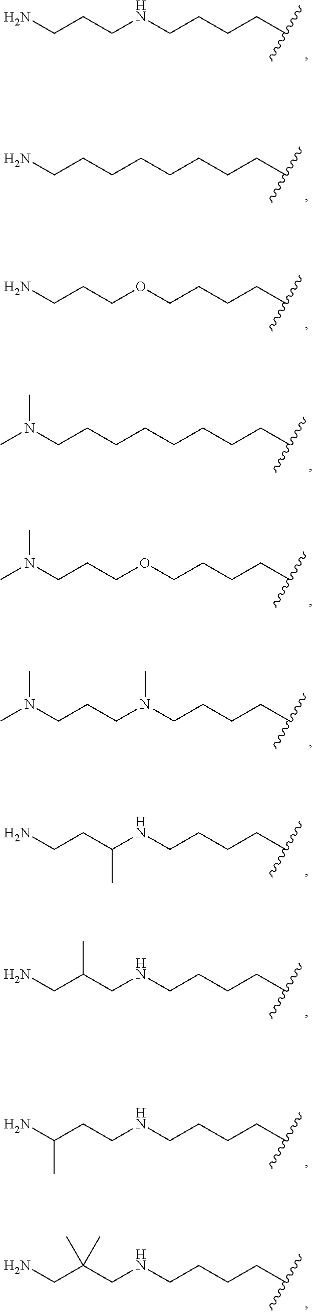

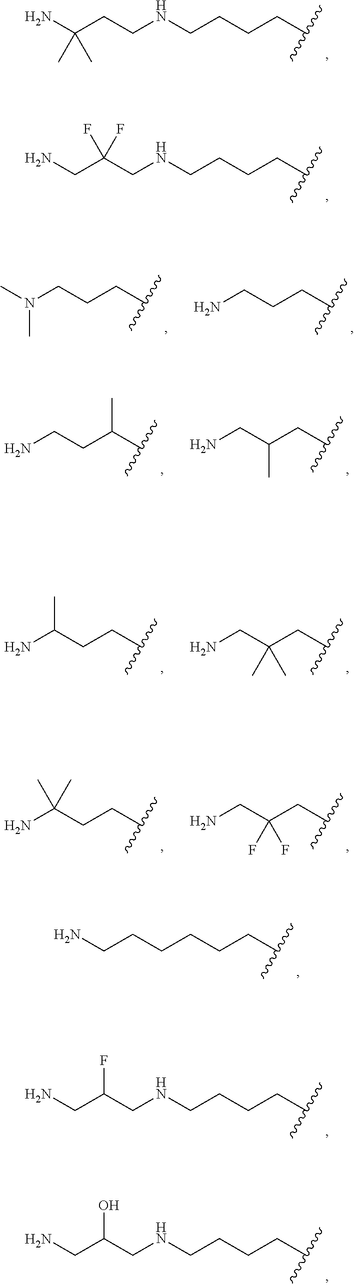

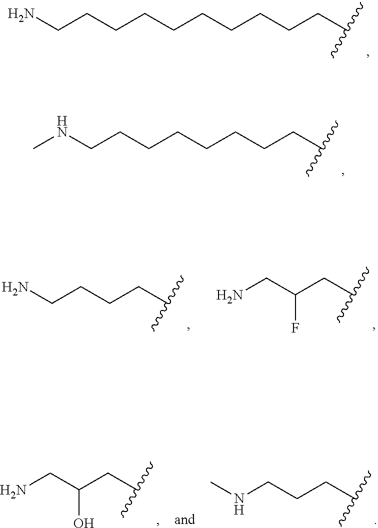

In some embodiments, one of R² and R³ is selected from

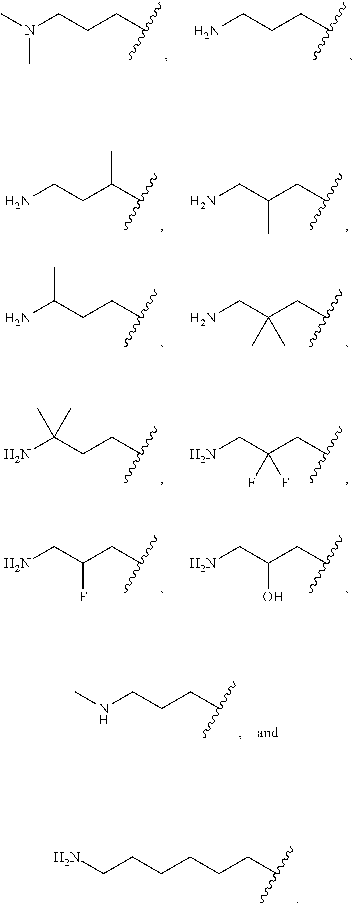

In some embodiments, one of R² and R³ is selected from

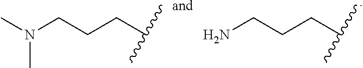

In some embodiments, one of R² and R³ is selected from

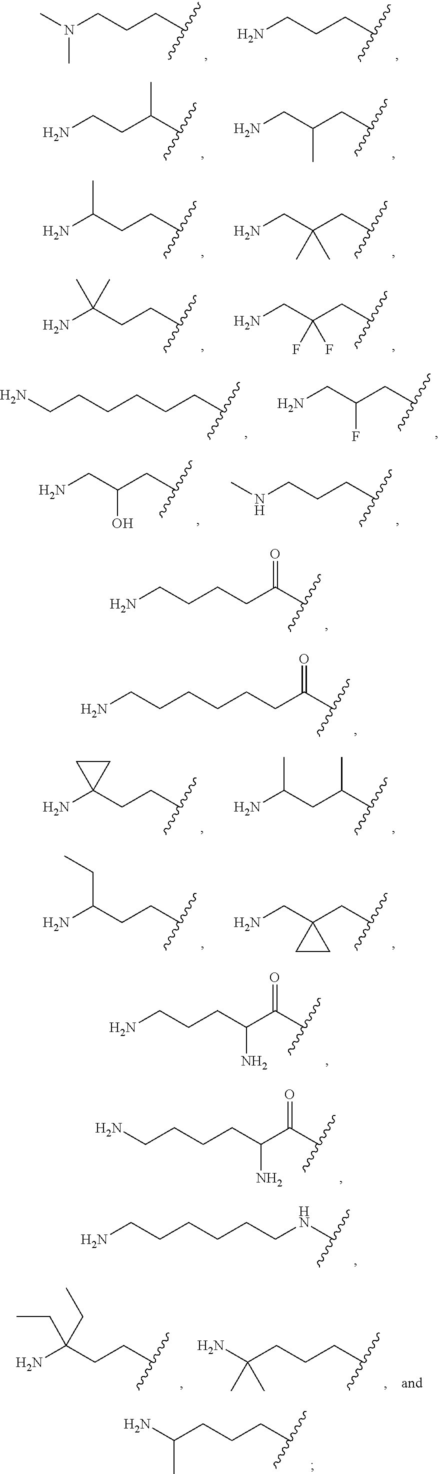

and the other of R² and R³ is selected from

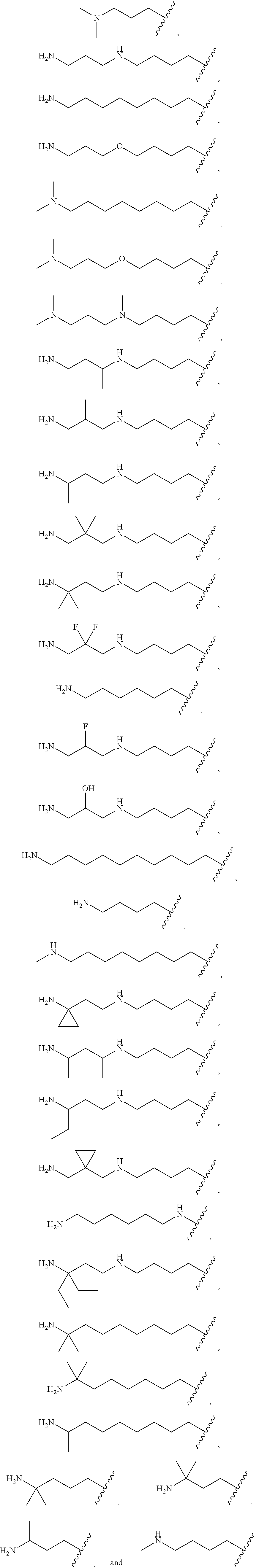

In some embodiments, one of R² and R³ is selected from

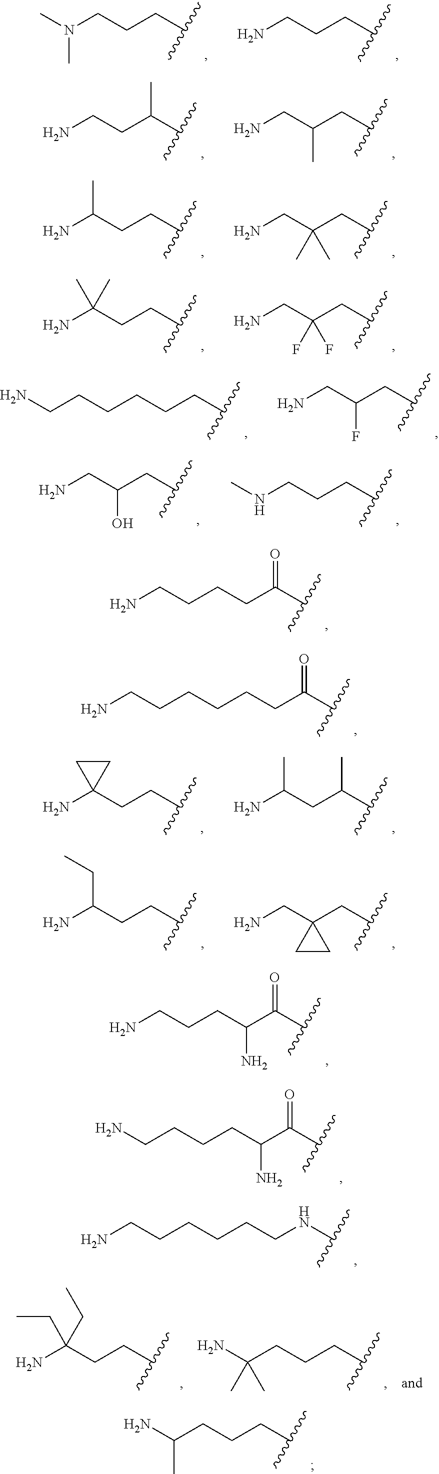

and the other of R² and R³ is selected from

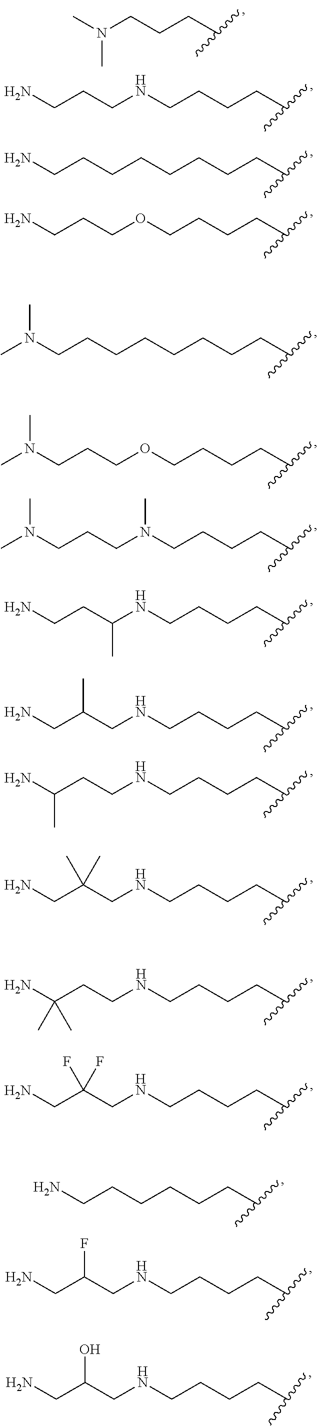

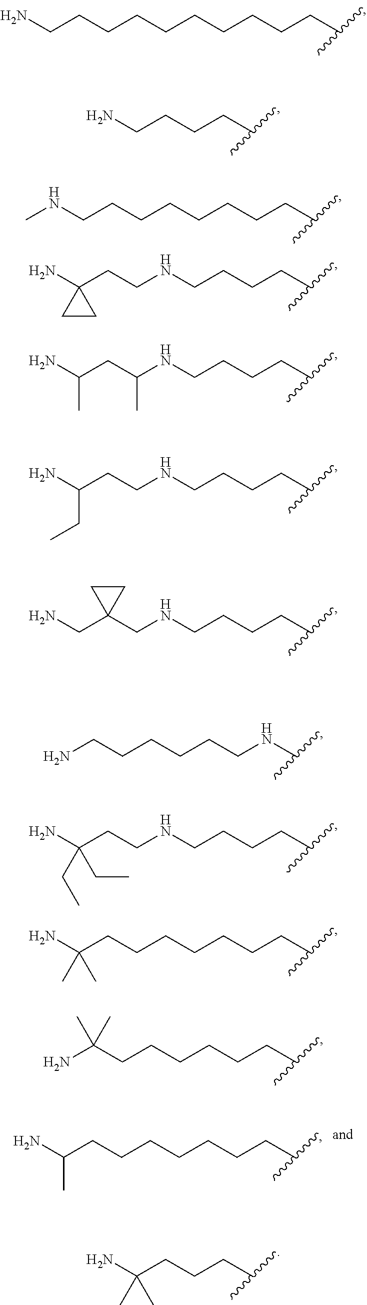

In some embodiments, one of R² and R³ is selected from

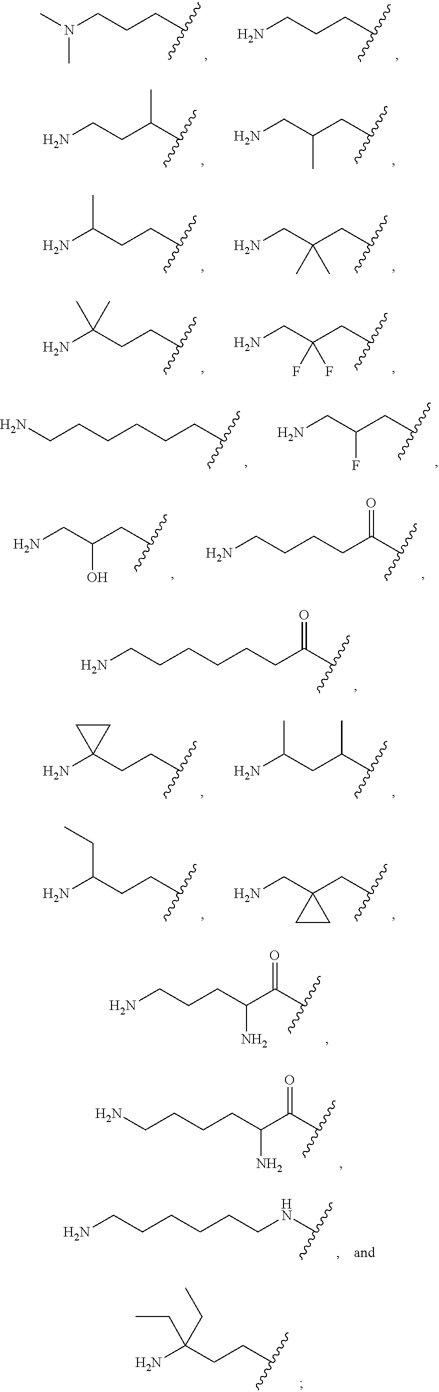

and the other of R² and R³ is selected from

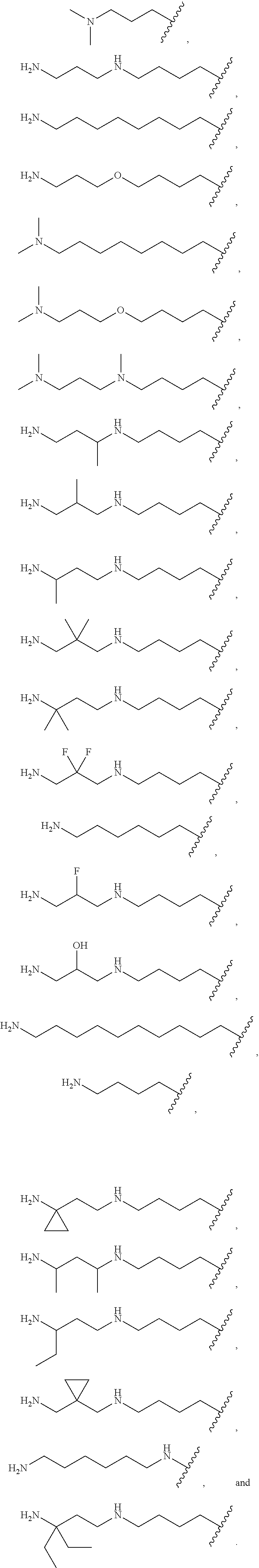

In some embodiments, one of R² and R³ is selected from

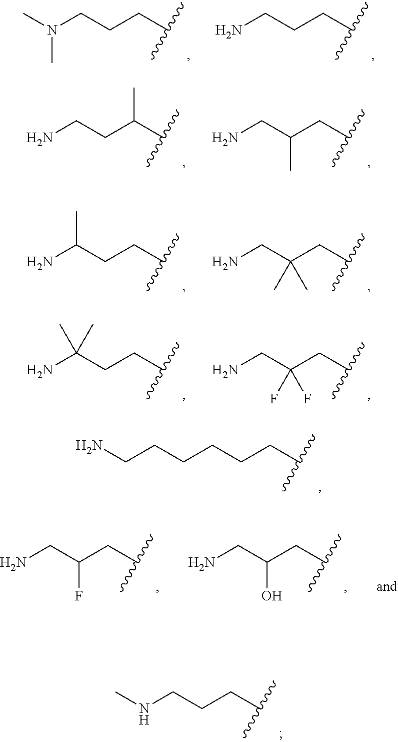

and the other of R² and R³ is selected from

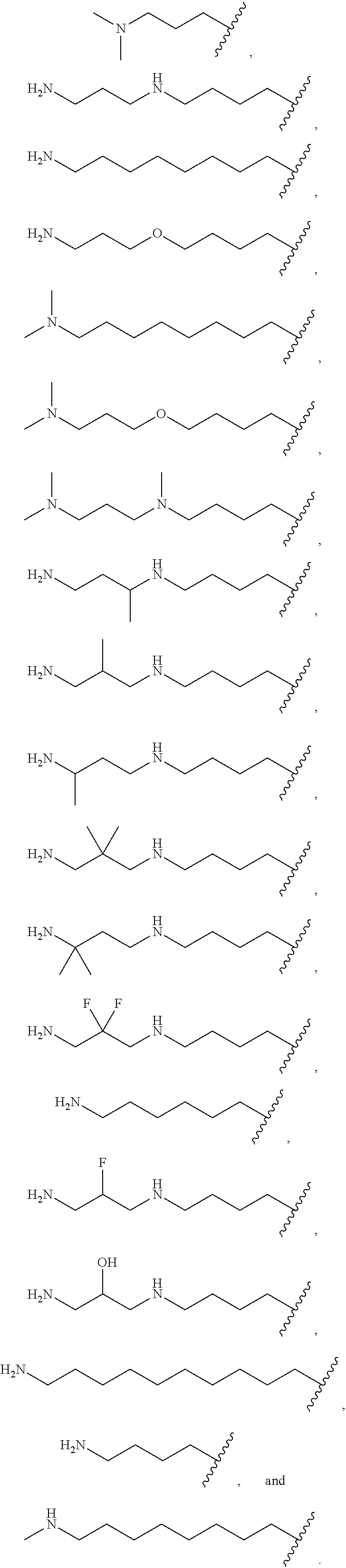

In some embodiments, R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 7-12 membered heterocycloalkyl group comprising 1, 2, or 3 ring-forming NR¹⁰ groups, wherein the 7-12 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 8-10 membered heterocycloalkyl group comprising 1, 2, or 3 ring-forming NR¹⁰ groups, wherein the 8-10 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form a 8-10 membered heterocycloalkyl group comprising 1, 2, or 3 ring-forming NCH₃ or NH groups, wherein the 8-10 membered heterocycloalkyl group is optionally substituted with 1, 2, or 3 substituents independently selected from C₁₋₄ alkyl, —NR⁸R⁹, OH, and halo.

In some embodiments, R² and R³ together with the N atom to which they are attached form an 8-10 membered heterocycloalkyl group comprising 1, 2, or 3 ring-forming NCH₃ or NH groups.

In some embodiments, R² and R³ together with the N atom to which they are attached form a heterocycloalkyl group of formula:

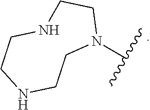 [SMILES: *N1CCNCCNCC1]

In some embodiments, R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR₈R₉, OH, and halo.

In some embodiments, R², R³, and R⁶, together with the atoms to which they are attached and any intervening atoms, form a 7-13 membered bridged heterocycloalkyl group optionally substituted with 1, 2, or 3 substituents independently selected from C₁₋₄ alkyl, —NR₈R₉, OH, and halo.

In some embodiments, R², R³, and R⁶, together with the atoms to which they are attached and any intervening atoms, form a 7-10 membered bridged heterocycloalkyl group optionally substituted with 1, 2, or 3 substituents independently selected from C₁₋₄ alkyl, —NR₈R₉, OH, and halo.

In some embodiments, R², R³, and R⁶, together with the atoms to which they are attached and any intervening atoms, form a 7-10 membered bridged heterocycloalkyl group.

In some embodiments, R², R³, and R⁶, together with the atoms to which they are attached and any intervening atoms, form a 7-18 membered bridged heterocycloalkyl group having the formula:

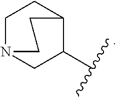 [SMILES: *C1CN2CCC1CC2]

In some embodiments, R⁴ and R⁵ are each independently H or C₁₋₄ alkyl. In some embodiments, R⁴ and R⁵ are each independently H or methyl. In some embodiments, both R⁴ and R⁵ are H. In some embodiments, both R⁴ and R⁵ are C₁₋₄ alkyl. In some embodiments, both R⁴ and R⁵ are methyl. In some embodiments, one of R⁴ and R⁵ is H and the other of R⁴ and R⁵ is C₁₋₄ alkyl. In some embodiments, one of R⁴ and R⁵ is H and the other of R⁴ and R⁵ is methyl.

In some embodiments, R⁶ and R⁷ are each independently H or C₁₋₄ alkyl. In some embodiments, R⁶ and R⁷ are each independently H or methyl. In some embodiments, both R⁶ and R⁷ are H. In some embodiments, both R⁶ and R⁷ are C₁₋₄ alkyl. In some embodiments, both R⁶ and R⁷ are methyl. In some embodiments, one of R⁶ and R⁷ is H and the other of R⁶ and R⁷ is C₁₋₄ alkyl. In some embodiments, one of R⁶ and R⁷ is H and the other of R⁶ and R⁷ is methyl.

In some embodiments, R⁸, R⁹, and R¹⁰ are each independently selected from H and methyl. In some embodiments, R⁸ and R⁹ are both H. In some embodiments, R⁸ and R⁹ are both C₁₋₄ alkyl. In some embodiments, R⁸ and R⁹ are both methyl. In some embodiments, one of R⁸ and R⁹ is H and the other of R⁸ and R⁹ is C₁₋₄ alkyl. In some embodiments, one of R⁸ and R⁹ is H and the other of R⁸ and R⁹ is methyl. In some embodiments, R¹⁰ is H or methyl. In some embodiments, R¹⁰ is H. In some embodiments, R¹⁰ is methyl.

In some embodiments, R^{a} and R^{b} together with the C atom to which they are attached form a C₃ cycloalkyl group such as cyclopropyl. In some embodiments, R^{a} and R^{b} together with the C atom to which they are attached form a C₄ cycloalkyl group such as cyclobutyl. In some embodiments, R^{a} and R^{b} together with the C atom to which they are attached form a C₅ cycloalkyl group such as cyclopentyl. In some embodiments, R^{a} and R^{b} together with the C atom to which they are attached form a C₆ cycloalkyl group such as cyclopentyl.

In some embodiments:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, OH, and halo, wherein at least one substituent is —NR⁸R⁹;
(ii) one non-terminal carbons of the C₂₋₂₀ alkyl are optionally replaced with O;
(iii) one non-terminal carbons of the C₂₋₂₀ alkyl are optionally replaced with NR¹⁰
(iv) one non-terminal carbons of the C₂₋₂₀ alkyl are optionally replaced with C(═O); and
(v) one non-terminal carbons of the C₂₋₂₀ alkyl are optionally replaced with CR^{a}R^{b} wherein R^{a} and R^{b} together with the C atom to which they are attached form a C₃₋₆ cycloalkyl group;
wherein R² and R³ are the same or different;
   or R² and R³ together with the N atom to which they are attached form a 7-18 membered heterocycloalkyl group comprising 2 ring-forming NR¹⁰ groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, 2, or 4; and
n is 0 or 1;
   wherein when j is 0, then 1 is 1,
wherein j and 1 are not both 0.

In some embodiments:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, OH, and halo, wherein at least one substituent is —NR⁸R⁹;
(ii) one non-terminal carbon of the C₂₋₂₀ alkyl are optionally replaced with O; and
(iii) one non-terminal carbon of the C₂₋₂₀ alkyl are optionally replaced with NR¹⁰;
wherein R² and R³ are the same or different;
or R² and R³ together with the N atom to which they are attached form a 7-18 membered heterocycloalkyl group comprising two ring-forming NR¹⁰ groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, 2, or 4; and
n is 0 or 1;
   wherein j and 1 are not both 0,
wherein when j is 0, then 1 is 1.

In some embodiments,
Z is N;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1 or 2 substituents independently selected from —NR⁸R⁹, OH, and halo, wherein at least one substituent is —NR⁸R⁹;
(ii) one non-terminal carbon of the C₂₋₂₀ alkyl is optionally replaced with O; and
(iii) one non-terminal carbon of the C₂₋₂₀ alkyl is optionally replaced with NR¹⁰;
wherein R² and R³ are the same or different;
or R² and R³ together with the N atom to which they are attached form a 7-18 membered heterocycloalkyl group comprising two ring-forming NR¹⁰ groups;
R⁴ is selected from H and C₁₋₄ alkyl;
R⁵, R⁶, and R⁷ are each H;
R⁸, R⁹, and R¹⁰ are each independently selected from H and C₁₋₄ alkyl;
j is 0 or 1;
k is 0, 1, 2, 3, or 4;
l is 0 or 1;
m is 0, 1, or 4; and
n is 0 or 1;
   wherein j and 1 are not both 0,
wherein when j is 0, then 1 is 1.

In some embodiments, the compound of Formula A1 is a compound of Formula A2:

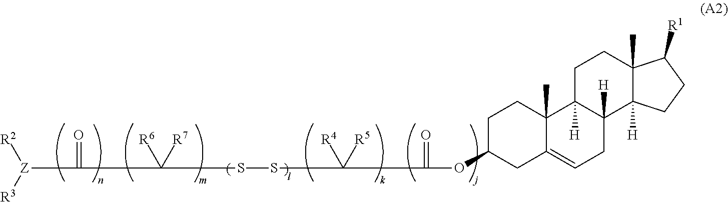

or a salt thereof.

In some embodiments, the compound of Formula A1 is selected from a compound of Formula A3-A15:

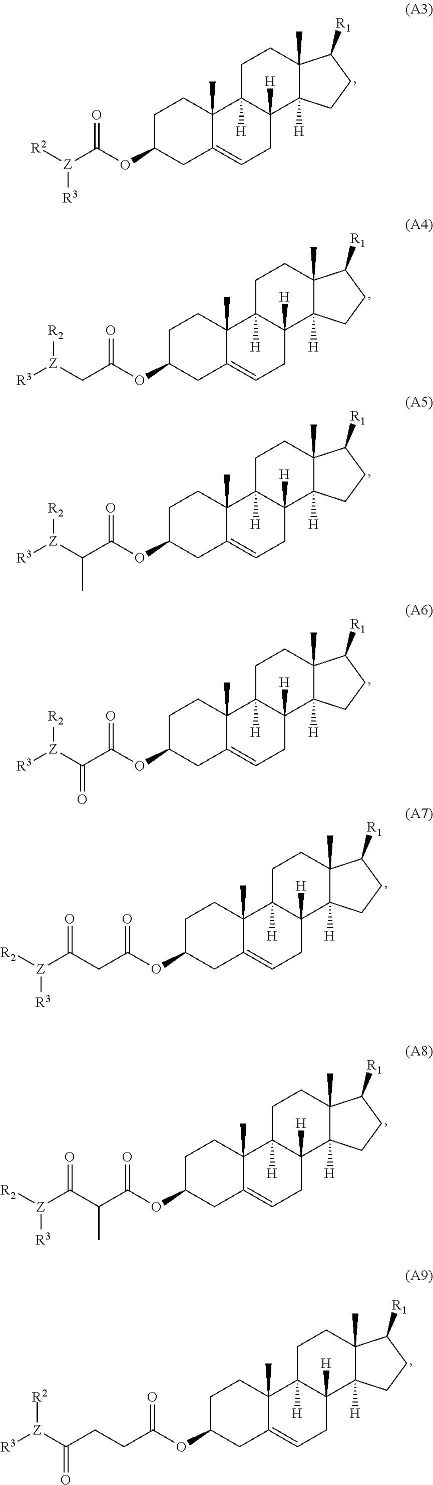

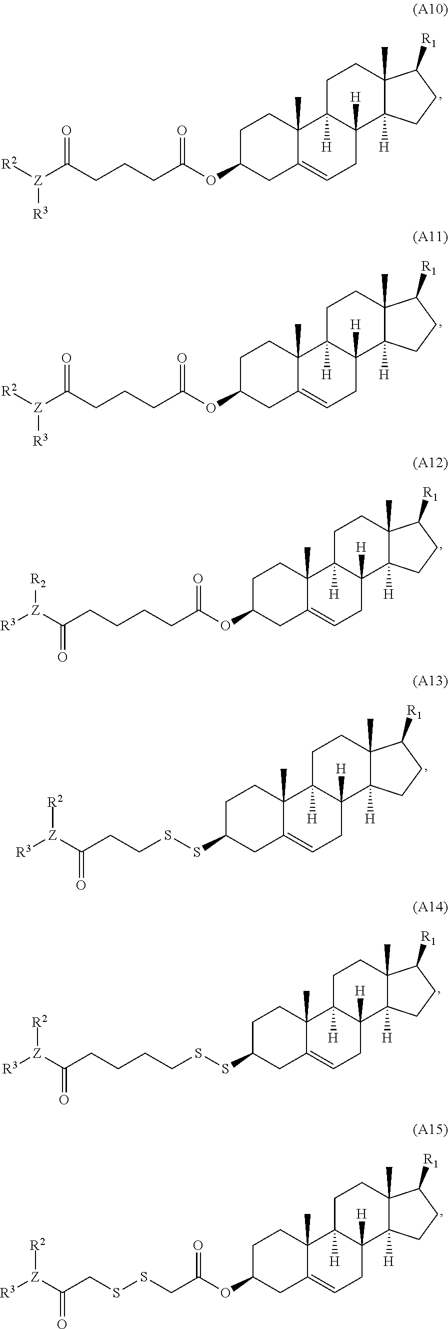

or a salt thereof.

In some embodiments, the compound of Formula A1 is selected from:

| No. | Structure |
|---|---|
| SA50 | 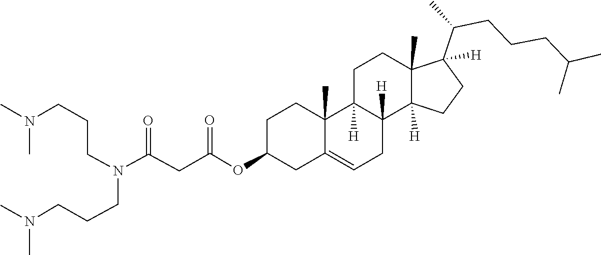 |
| SA51 | 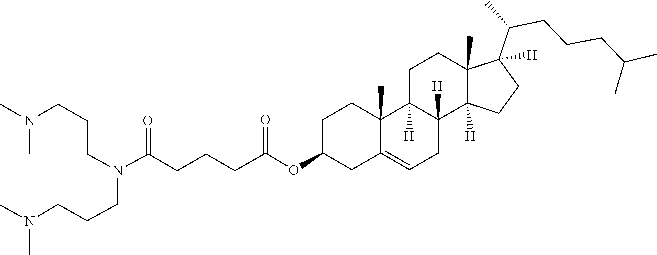 |
| SA56 | 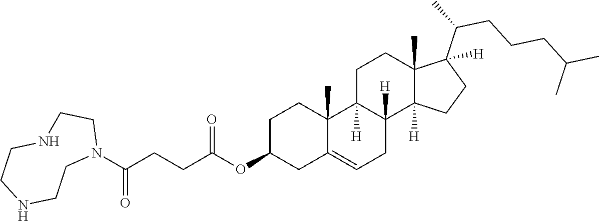 |
| SA57 | 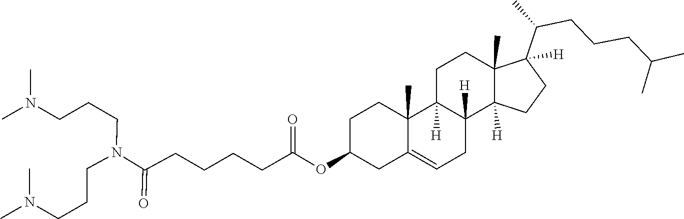 |
| SA58 | 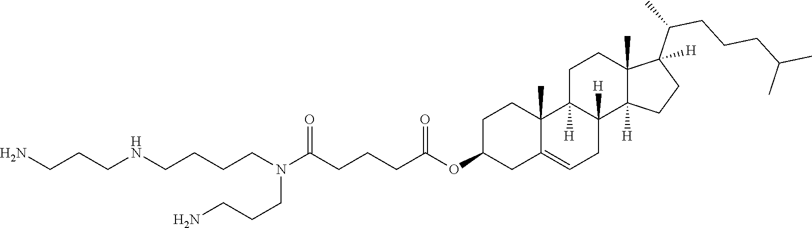 |
| SA59 | 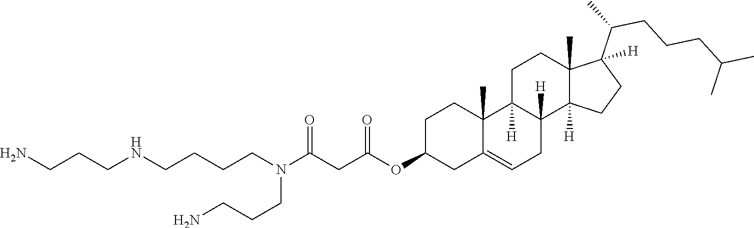 |
| SA60 | 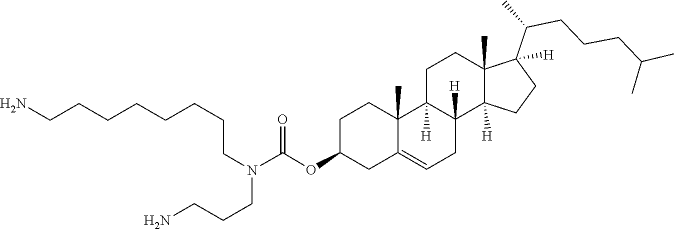 |
| SA61 | 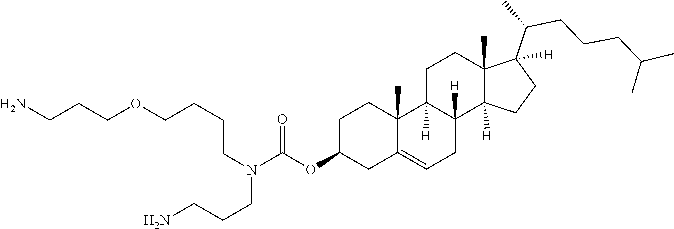 |
| SA62 | 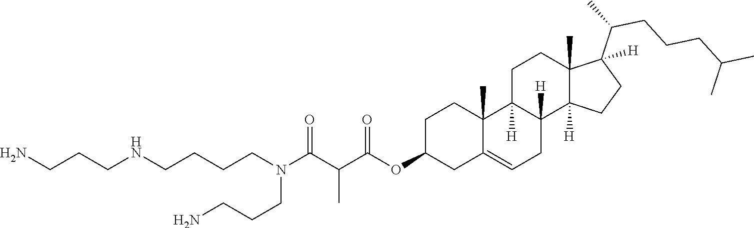 |
| SA63 | 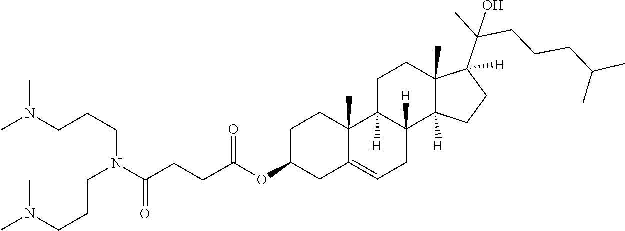 |
| SA64 | 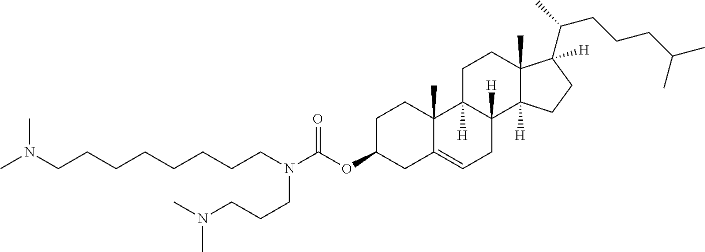 |
| SA65 | 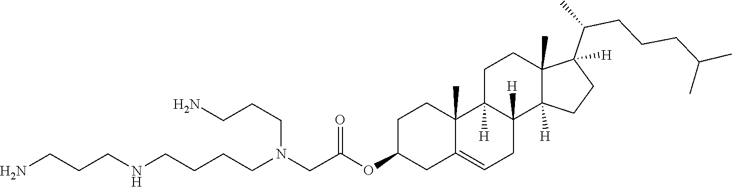 |
| SA66 | 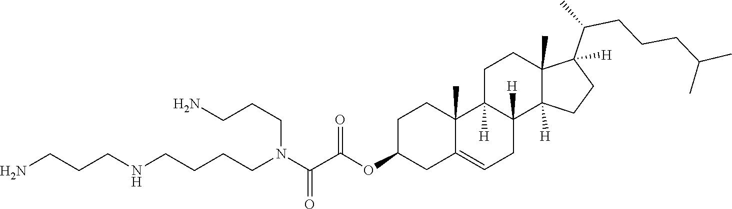 |
| SA67 | 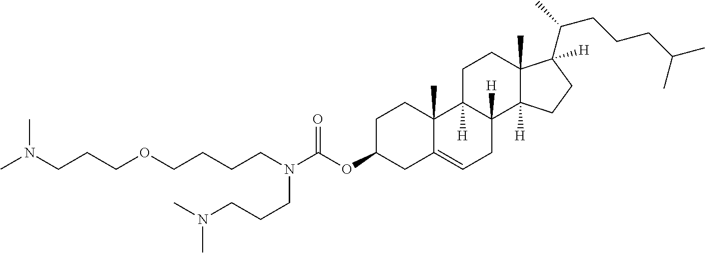 |
| SA68 | 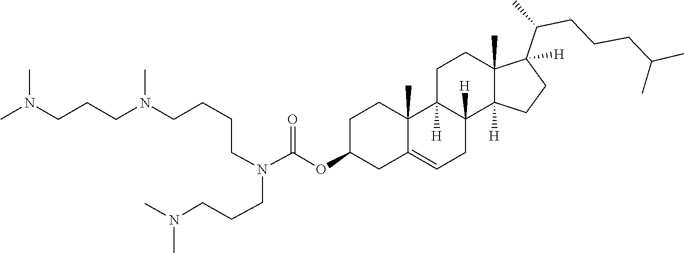 |
| SA69 | 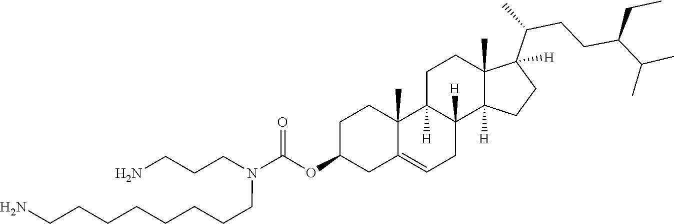 |
| SA70 | 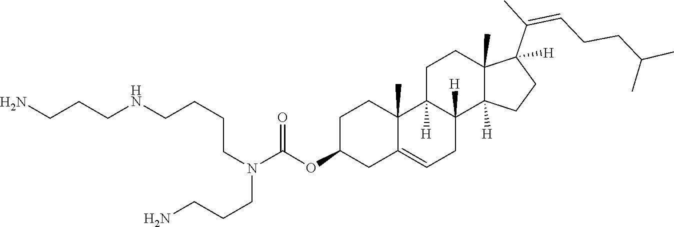 |
| SA71 | 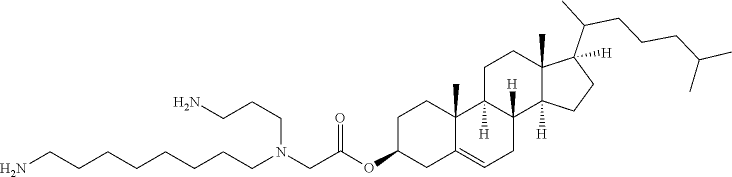 |
| SA72 | 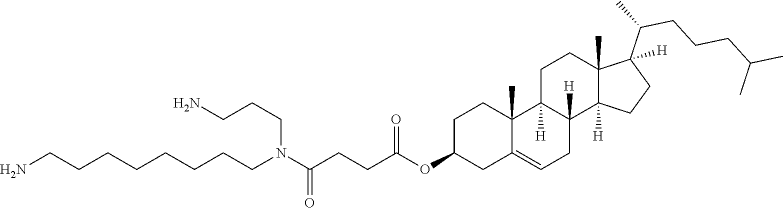 |
| SA73 | 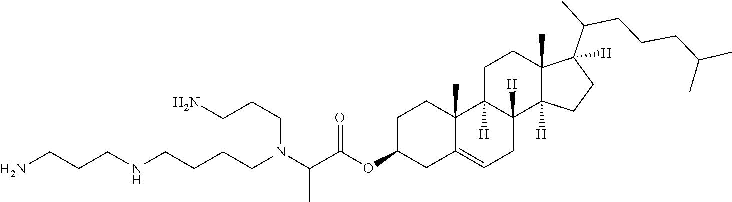 |
| SA74 | 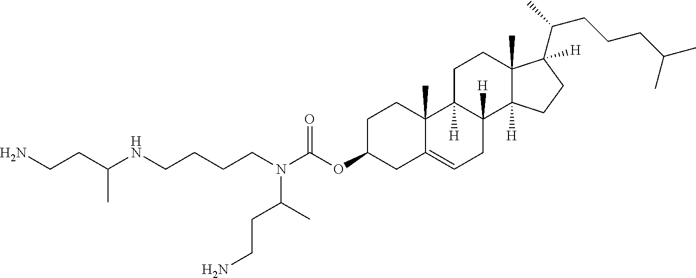 |
| SA75 | 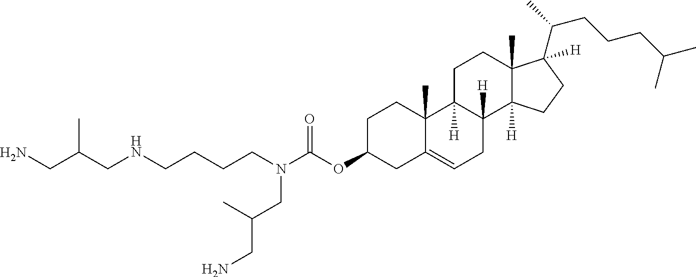 |
| SA76 | 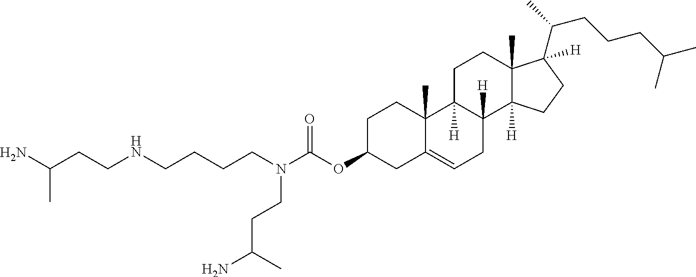 |
| SA77 | 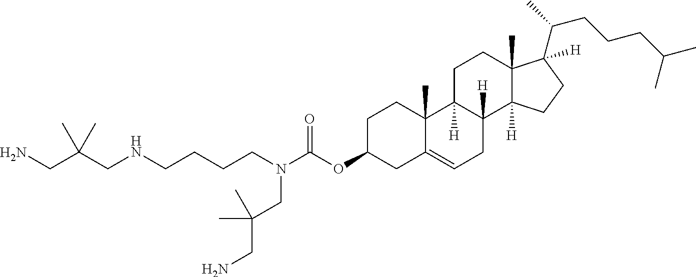 |
| SA78 | 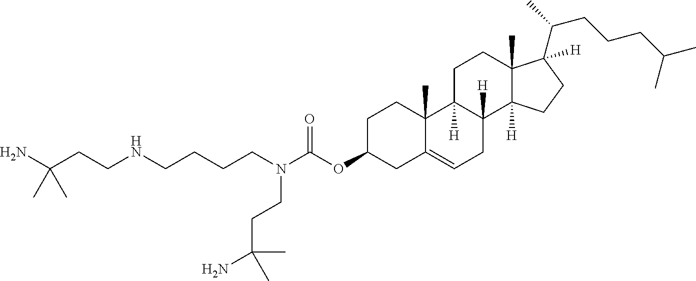 |
| SA79 | 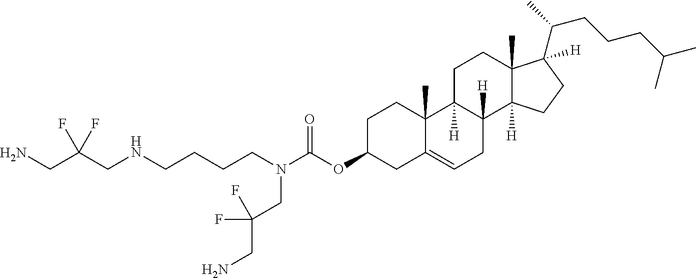 |
| SA81 | 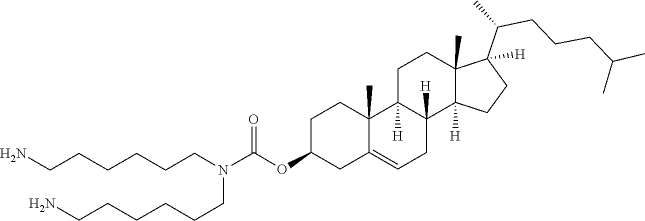 |
| SA82 | 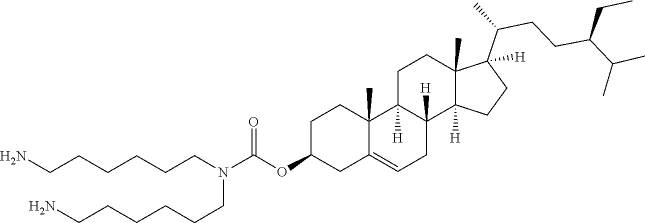 |
| SA83 | 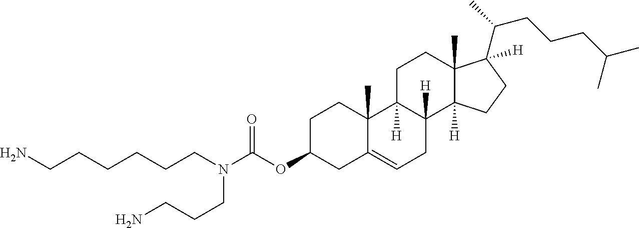 |
| SA84 | 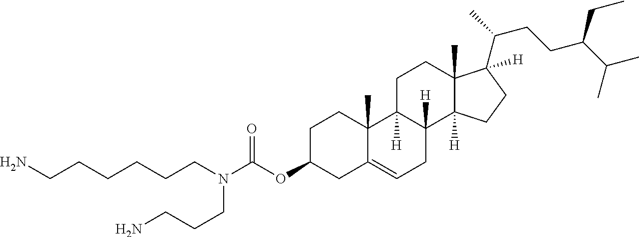 |
| SA87 | 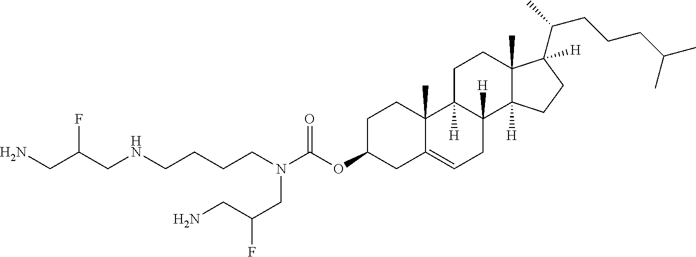 |
| SA88 | 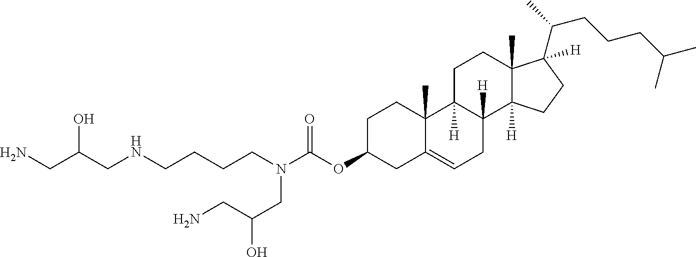 |
| SA89 | 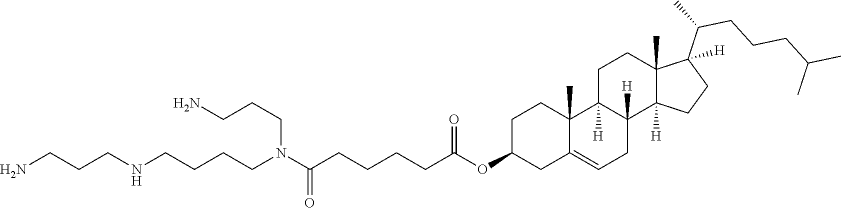 |
| SA90 | 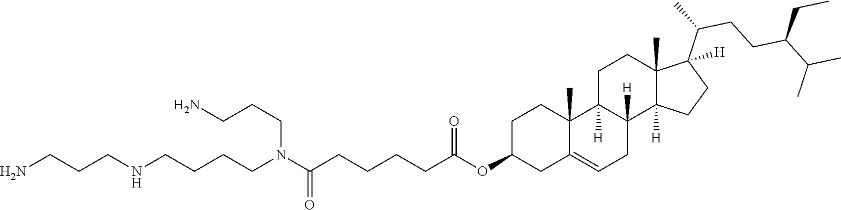 |
| SA95 | 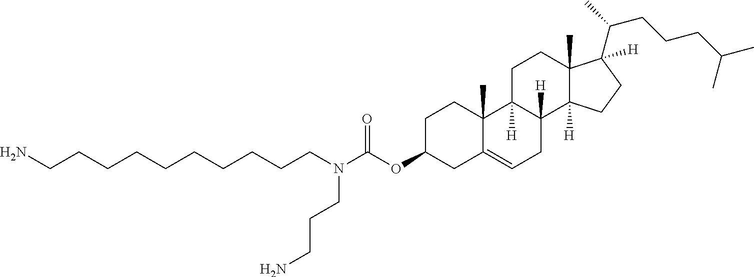 |
| SA96 | 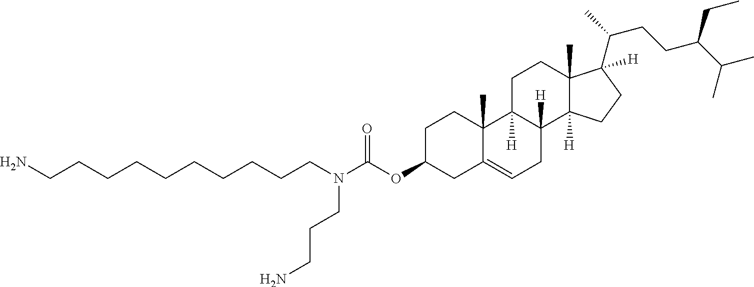 |
| SA97 | 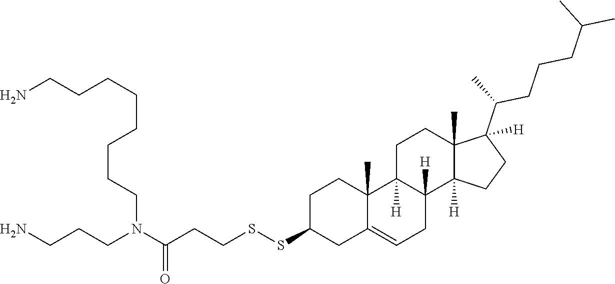 |
| SA98 | 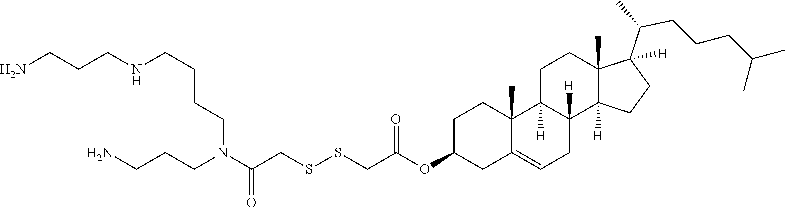 |
| SA110 | 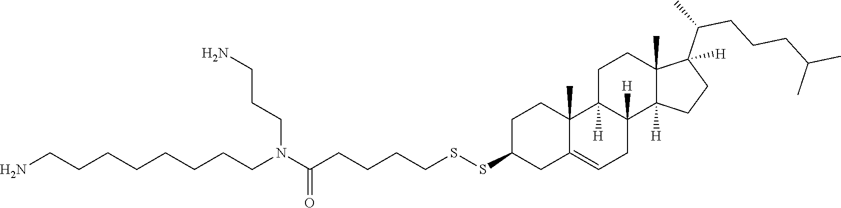 |
| SA111 | 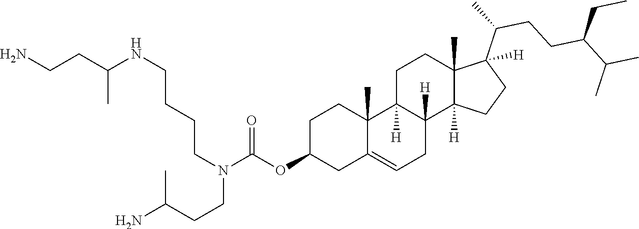 |
| SA113 | 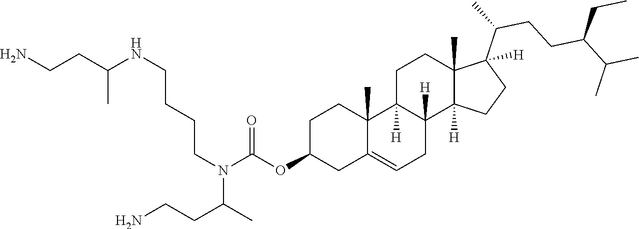 |
| SA114 | 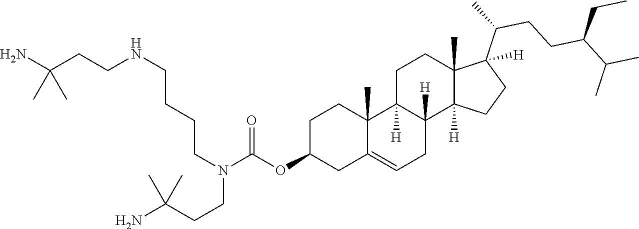 |
| SA116 | 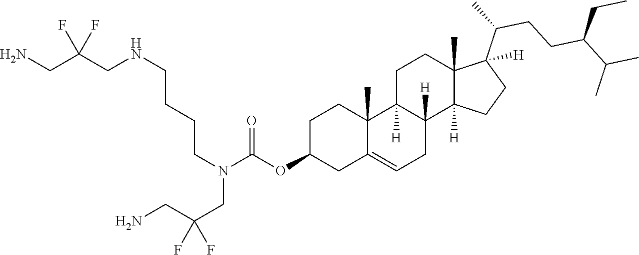 |
| SA117 | 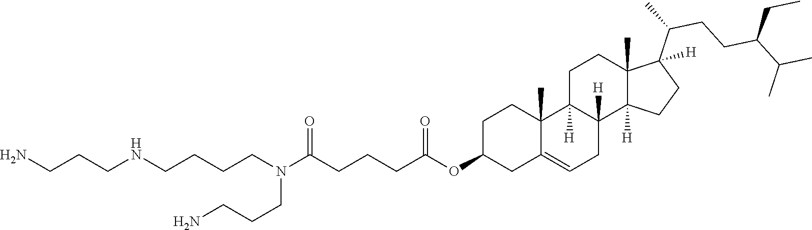 |
| SA119 | 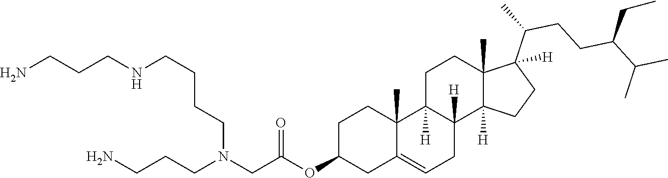 |
| SA120 | 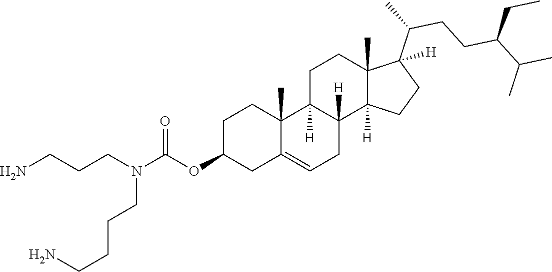 |

or a salt oaf any of the aforementioned.

In some embodiments, the compound of Formula A1 is selected from:

| No. | Structure |
|---|---|
| SA118 | 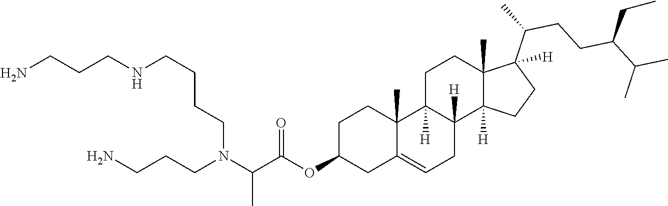 |
| SA121 | 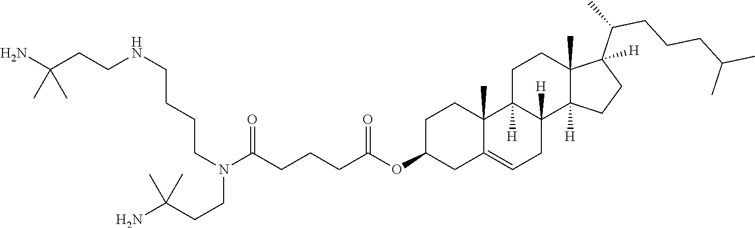 |
| SA122 | 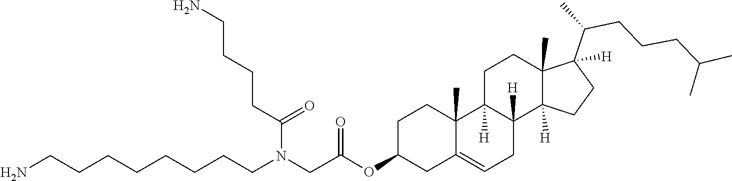 |
| SA123 | 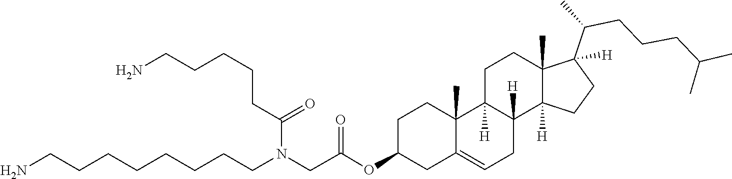 |
| SA124 | 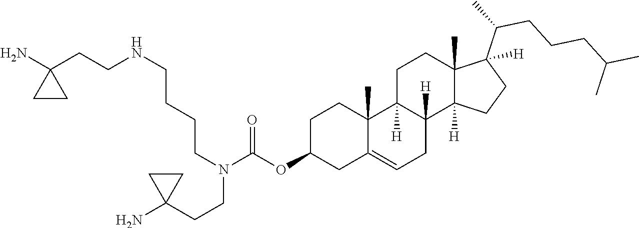 |
| SA125 | 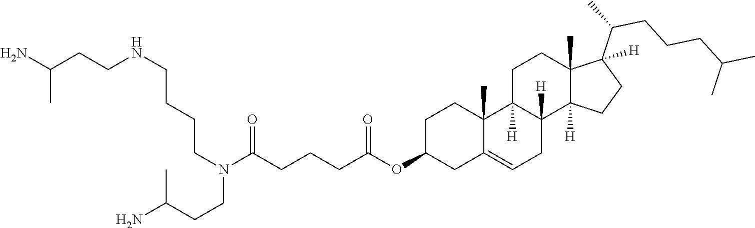 |
| SA126 | 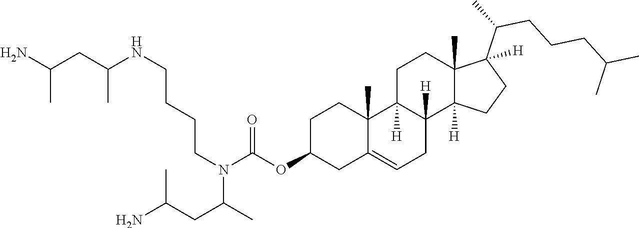 |
| SA127 | 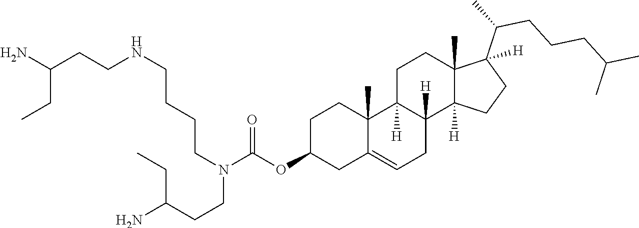 |
| SA128 | 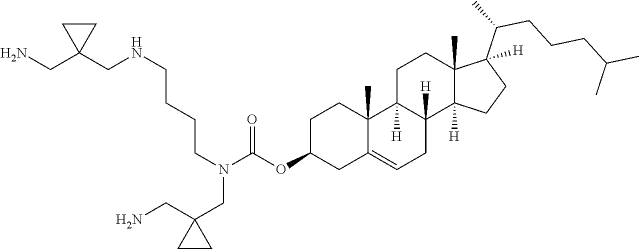 |
| SA129 | 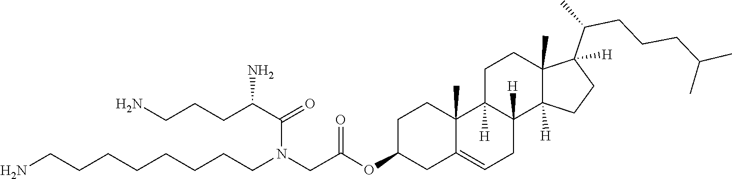 |
| SA130 | 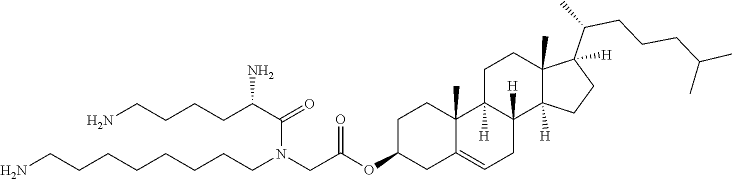 |
| SA131 | 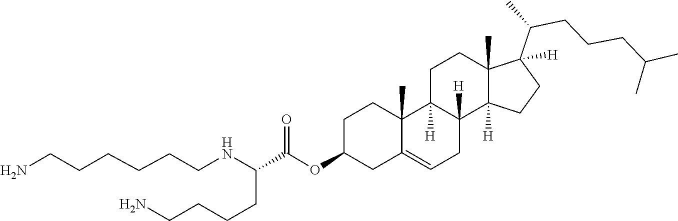 |
| SA132 | 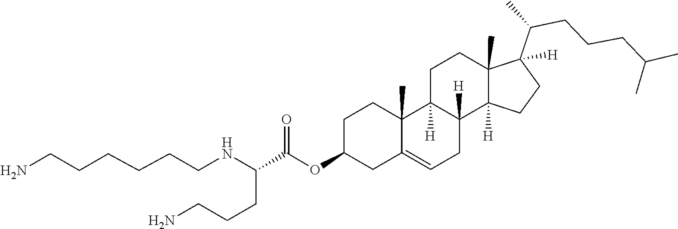 |
| SA133 | 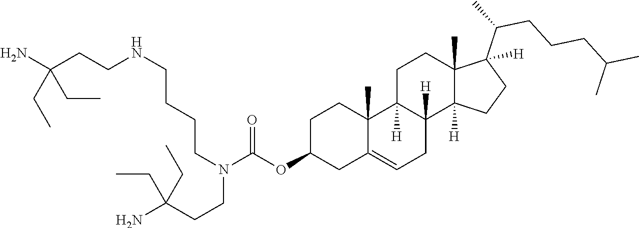 |
| SA134 | 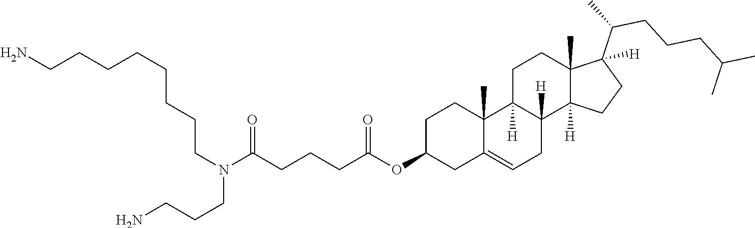 |
| SA135 | 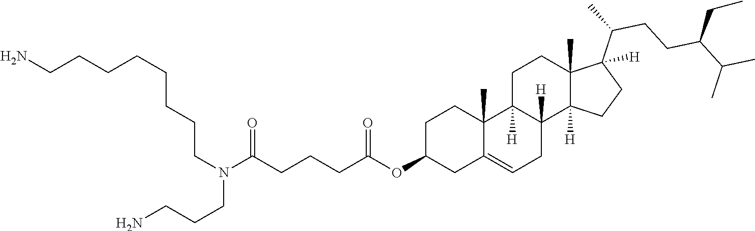 |
| SA136 | 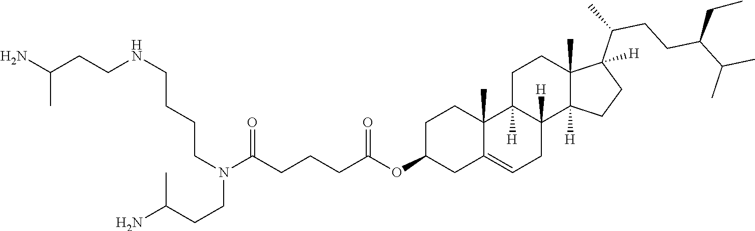 |
| SA137 | 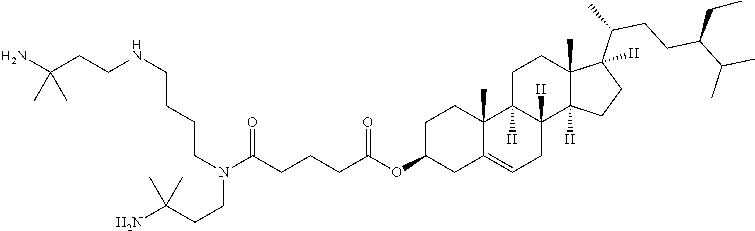 |
| SA138 | 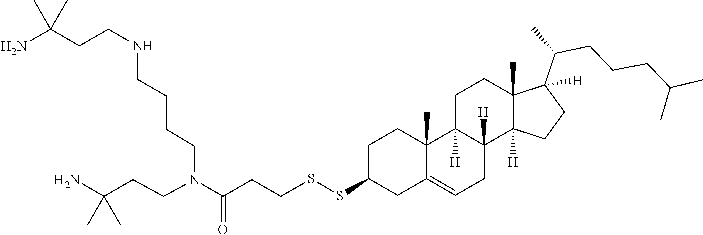 |

or a salt of any of the aforementioned.

In some embodiments, the compound of Formula A1 is selected from:

| No. | Structure |
|---|---|
| SA139 | 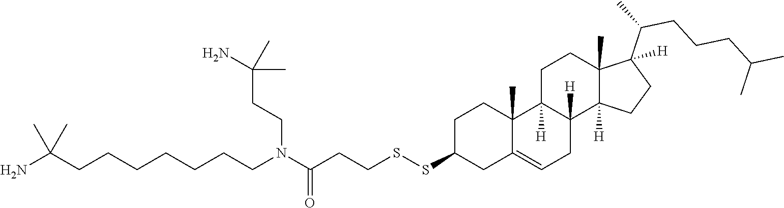 |
| SA141 | 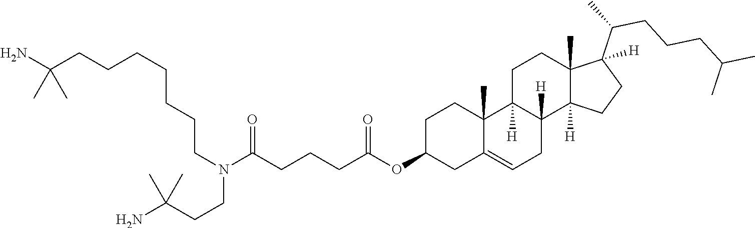 |
| SA142 | 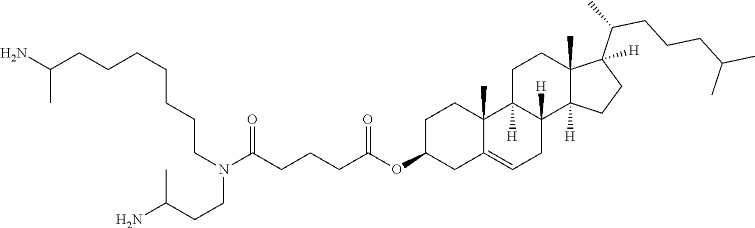 |
| SA144 | 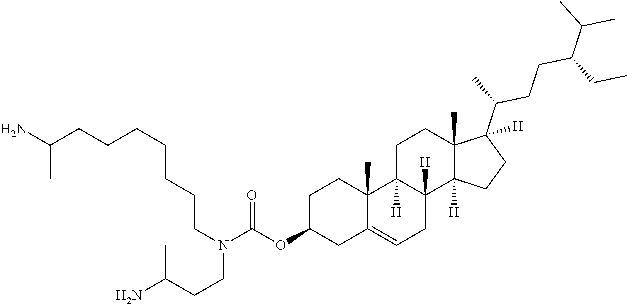 |
| SA145 | 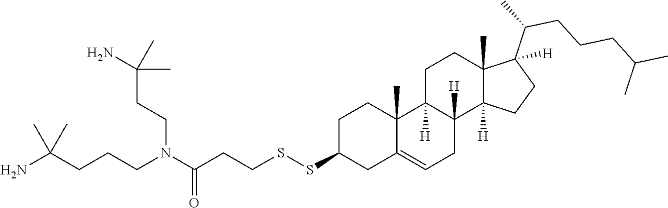 |
| SA149 | 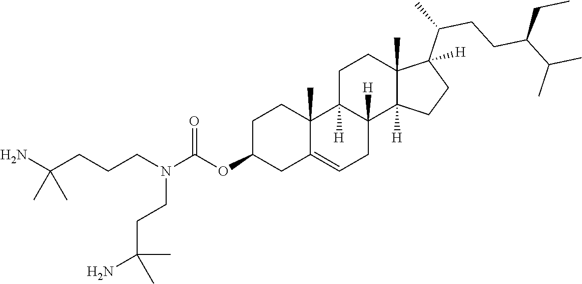 |
| SA151 | 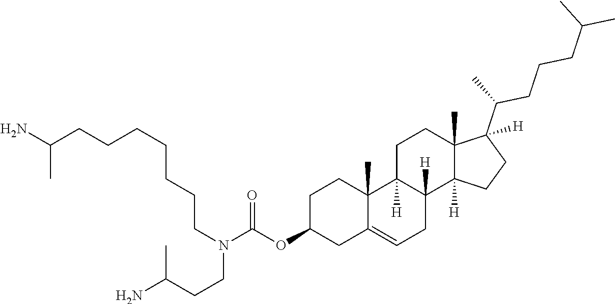 |
| SA152 | 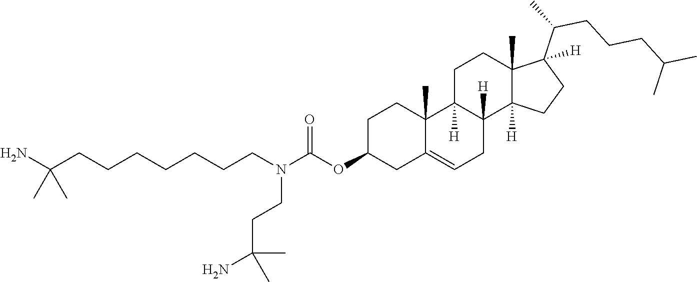 |
| SA153 | 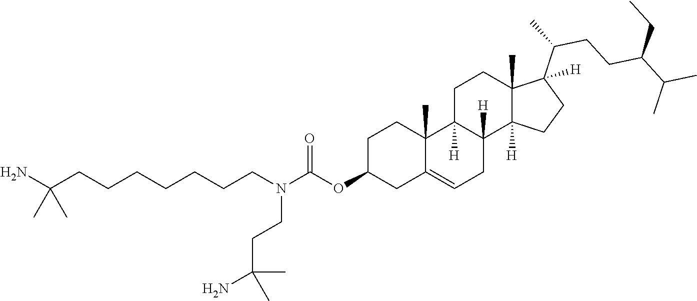 |
| SA154 | 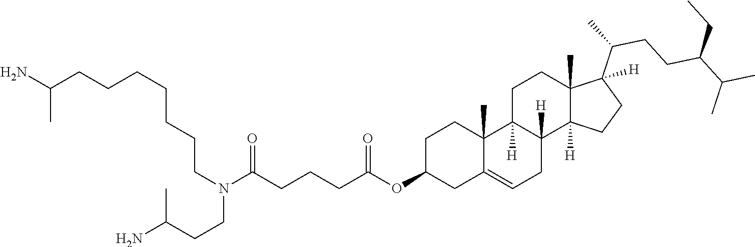 |
| SA155 | 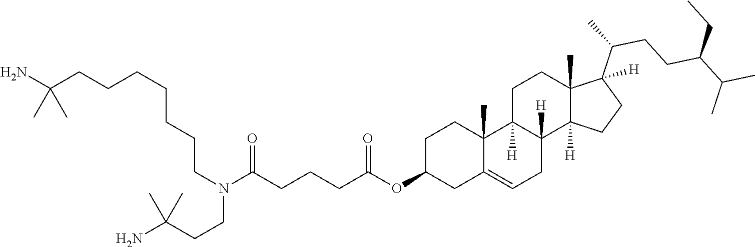 |
| SA156 | 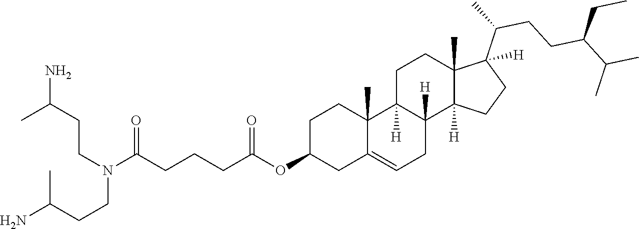 |
| SA157 | 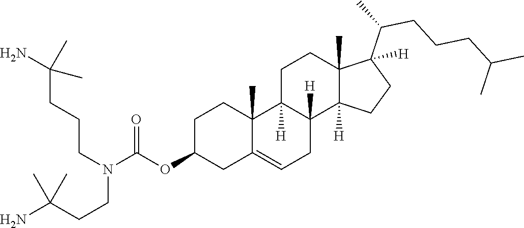 |
| SA158 | 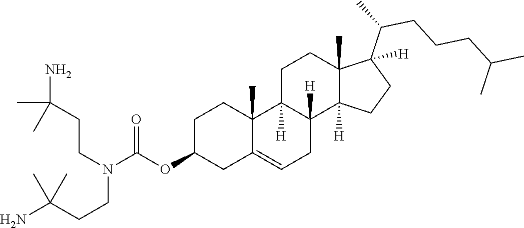 |
| SA159 | 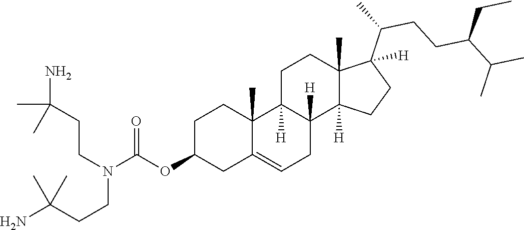 |
| SA160 | 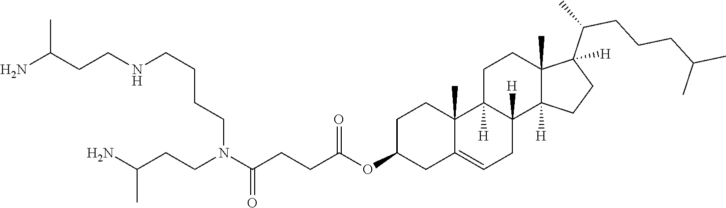 |
| SA161 | 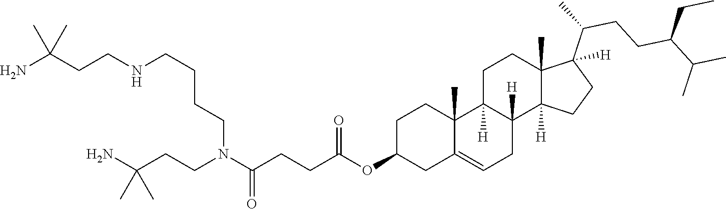 |
| SA162 | 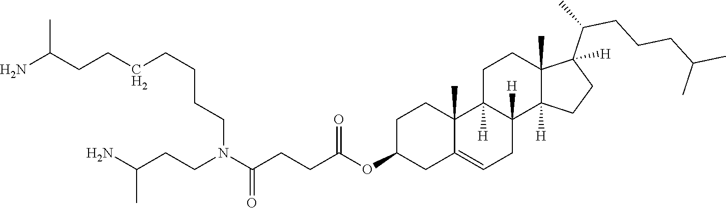 |
| SA163 | 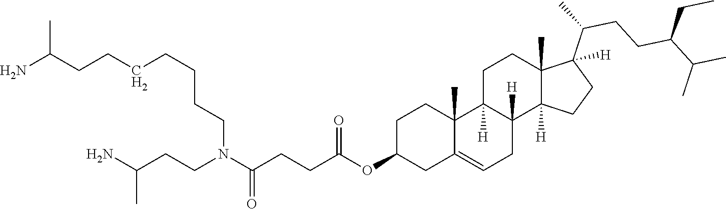 |
| SA164 | 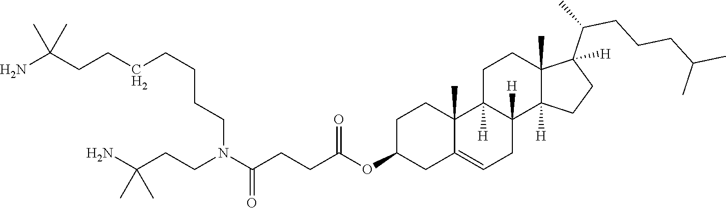 |
| SA165 | 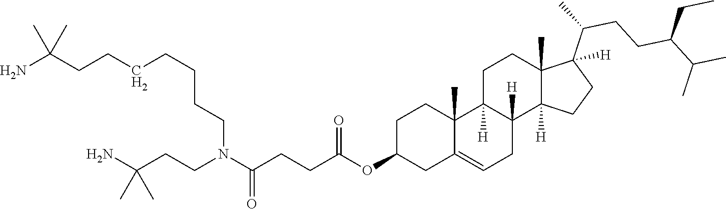 |
| SA166 | 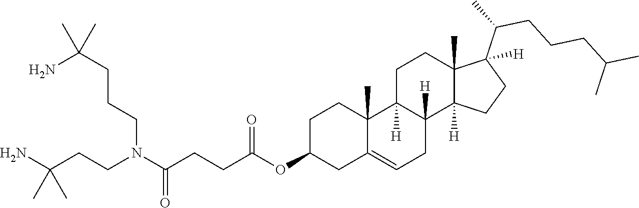 |
| SA167 | 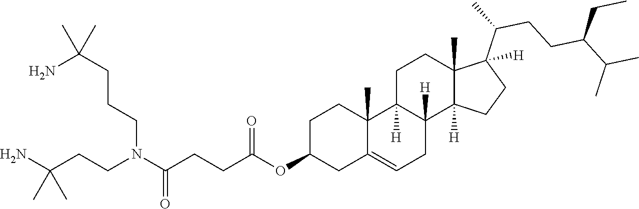 |
| SA168 | 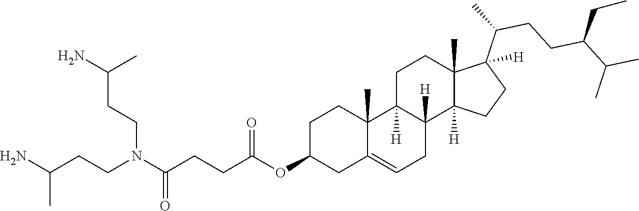 |
| SA169 | 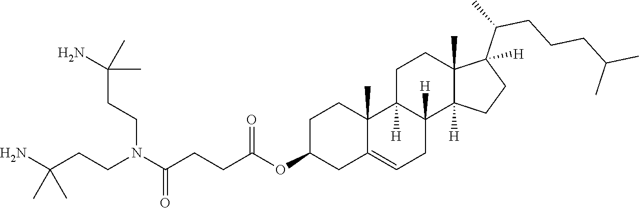 |
| SA170 | 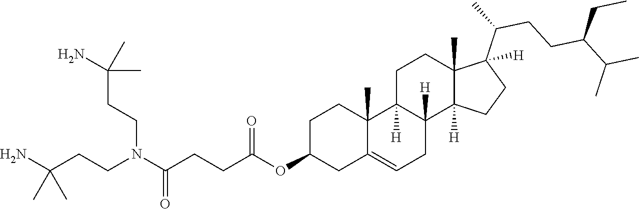 |
| SA171 | 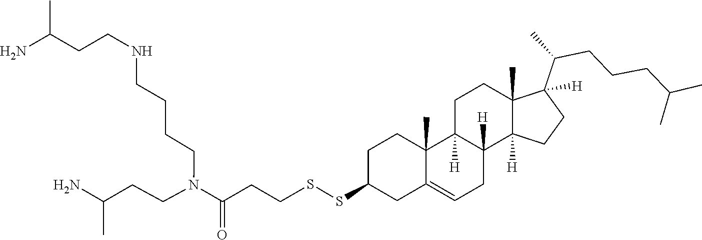 |
| SA172 | 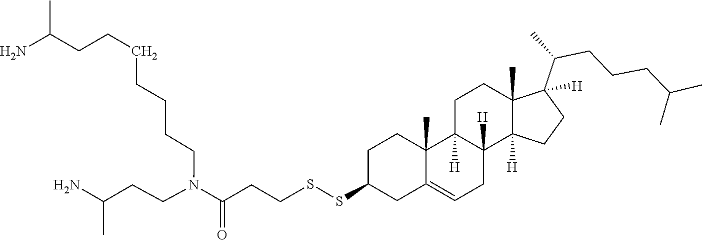 |
| SA173 | 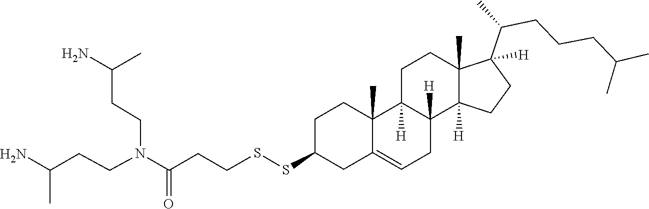 |
| SA174 | 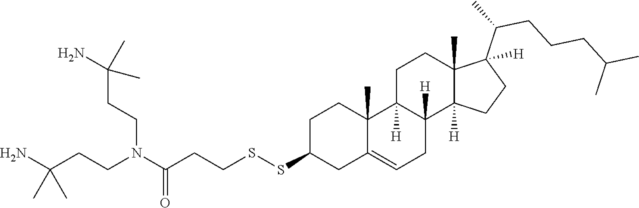 |
| SA175 | 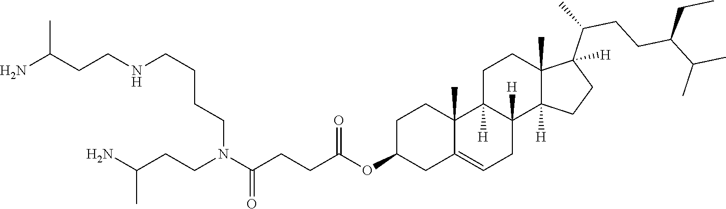 |
| SA176 | 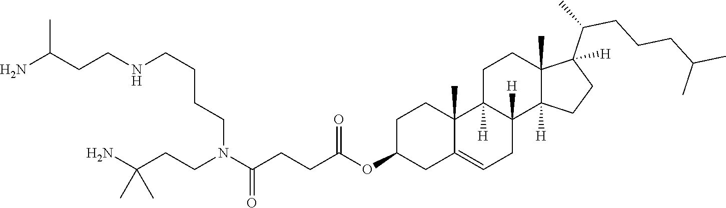 |
| SA177 | 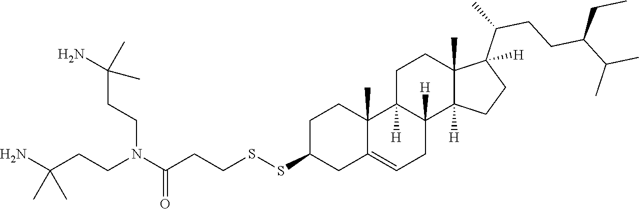 |
| SA178 | 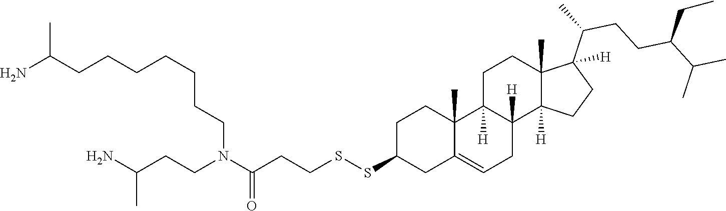 |
| SA179 | 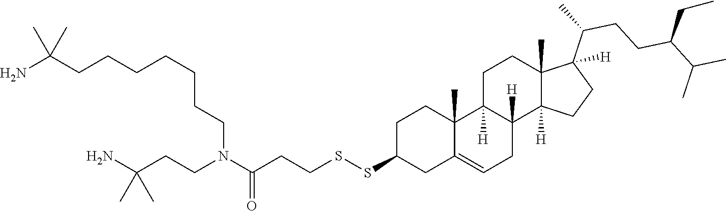 |
| SA180 | 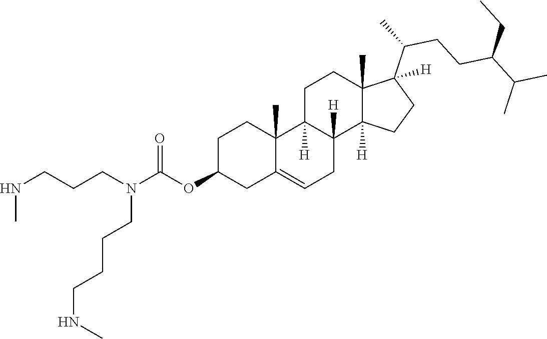 |
| SA181 | 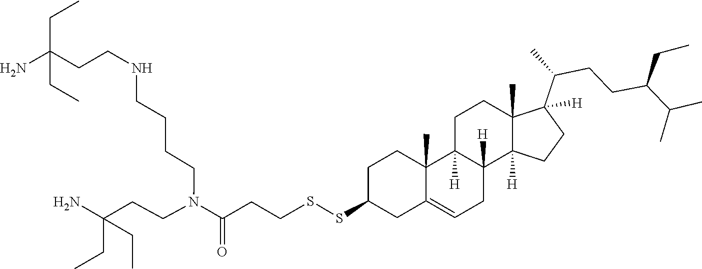 |
| SA182 | 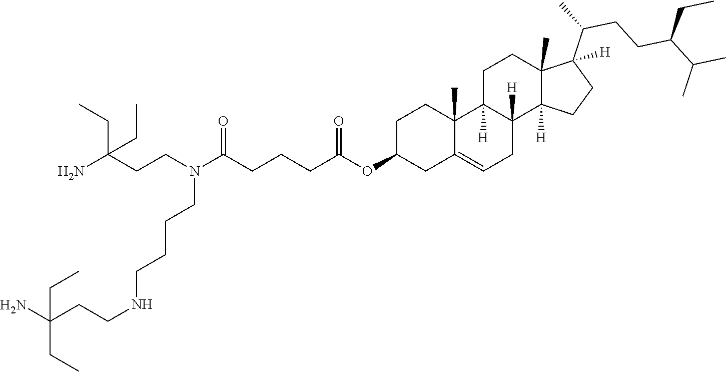 |
| SA183 | 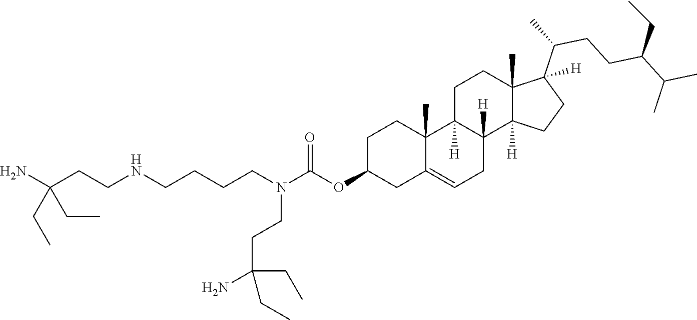 |
| SA184 | 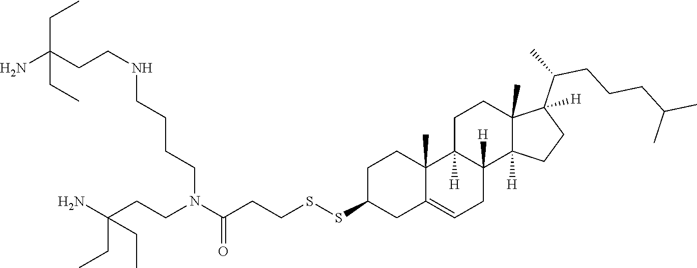 |
| SA185 | 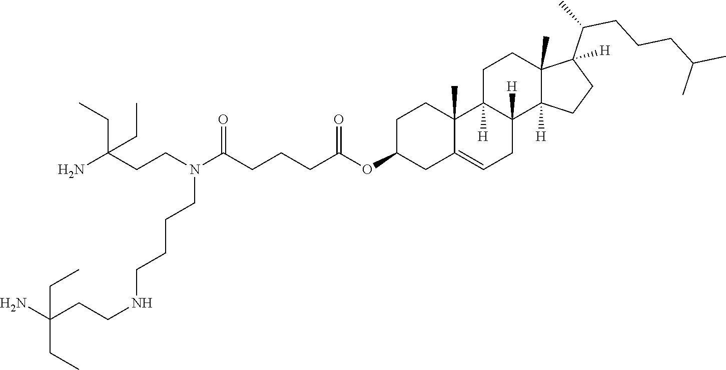 |

or a salt of any of the aforementioned.

It is appreciated that certain features described herein, which are, for clarity, described in the context of separate embodiments, can also be provided in combination in a single embodiment. Conversely, various features described herein which are, for brevity, described in the context of a single embodiment, can also be provided separately or in any suitable subcombination.

### Lipid Nanoparticle Compositions

Further provided is a lipid nanoparticle (LNP) composition comprising a lipid amine disclosed herein, such as a lipid amine of Formula A1. In some embodiments, the lipid nanoparticle composition further comprises, in addition to the lipid amine, at least one of an ionizable lipid, a phospholipid, a structural lipid, and a PEG-lipid. In some embodiments, the lipid nanoparticles of the lipid nanoparticle composition are loaded with payload. In some embodiments, the lipid amine is disposed primarily on the outer surface of the lipid nanoparticles of the lipid nanoparticle composition. In some embodiments, the lipid nanoparticle composition has a greater than neutral zeta potential at physiologic pH.

In some embodiments, the lipid nanoparticle composition comprises:
(i) an ionizable lipid,
(ii) a phospholipid,
(iii) a structural lipid,
(iv) optionally a PEG-lipid,
(v) optionally a payload for delivery into a cell, and
(vi) a lipid amine as disclosed herein, such as the lipid amine of Formula A1.

The lipid nanoparticle compositions can further comprise additional components, including but not limited to, helper lipids, stabilizers, salts, buffers, and solvents. The helper lipid is a non-cationic lipid. The helper lipid may comprise at least one fatty acid chain of at least eight carbons and at least one polar headgroup moiety. In some embodiments, the lipid nanoparticle core has a neutral charge at a neutral pH.

In some embodiments, the weight ratio of the lipid amine to payload in the lipid nanoparticle compositions is about 0.1:1 to about 15:1, about 0.2:1 to about 10:1, about 1:1 to about 10:1, about 1:1 to about 8:1, about 1:1 to about 7:1, about 1:1 to about 6:1, about 1:1 to about 5:1, about 1:1 to about 4:1, or about 1.25:1 to about 3.75:1. In some embodiments, a weight ratio of the lipid amine to payload is about 1.25:1, about 2.5:1, or about 3.75:1. In some embodiments, a molar ratio of the lipid amine to payload is about 0.1:1 to about 20:1, about 1.5:1 to about 10:1, about 1.5:1 to about 9:1, about 1.5:1 to about 8:1, about 1.5:1 to about 7:1, about 1.5:1 to about 6:1, or about 1.5:1 to about 5:1. In some embodiments, a molar ratio of the lipid amine to payload is about 1.5:1, about 2:1, about 3:1, about 4:1, or about 5:1.

In some embodiments, the lipid nanoparticle composition is characterized as having a zeta potential of about 5 mV to about 20 mV. In some embodiments, the lipid nanoparticle composition has a zeta potential of about 5 mV to about 15 mV. In some embodiments, the lipid nanoparticle composition has a zeta potential of about 5 mV to about 10 mV. Zeta potential measures the surface charge of colloidal dispersions. The magnitude of the zeta potential indicates the degree of electrostatic repulsion between adjacent, similarly charged particles in the dispersion. Zeta potential can be measured on a Wyatt Technologies Mobius Zeta Potential instrument. This instrument characterizes the mobility and zeta potential by the principle of “Massively Parallel Phase Analysis Light Scattering” or MP-PALS. This measurement is more sensitive and less stress inducing than ISO Method 13099-1:2012 which only uses one angle of detection and required higher voltage for operation. In some embodiments, the zeta potential of the herein described empty lipid nanoparticle compositions lipid is measured using an instrument employing the principle of MP-PALS. Zeta potential can be measured on a Malvern Zetasizer (Nano ZS).

In some embodiments, greater than about 80%, greater than about 90%, or greater than about 95% of the lipid amine is on the surface on the lipid nanoparticles of the lipid nanoparticle composition.

In some embodiments, the lipid nanoparticle composition has a polydispersity value of less than about 0.4, less than about 0.3 or less than about 0.2. In some embodiments, the LNP has a polydispersity value of about 0.1 to about 1, about 0.1 to about 0.5 or about 0.1 to about 0.3.

In some embodiments, the lipid nanoparticles of the lipid nanoparticle composition has a mean diameter of about 40 nm to about 150 nm, about 50 nm to about 100 nm, about 60 nm to about 120 nm, about 60 nm to about 100 nm, or about 60 nm to about 80 nm.

In some embodiments, a general polarization of laurdan of the lipid nanoparticles of the lipid nanoparticle composition is greater than or equal to about 0.6. In some embodiments, the LNP has a d-spacing of greater than about 6 nm or greater than about 7 nm.

In some embodiments, at least about 50%, at least about 75%, at least about 90%, at least about 95% of the lipid nanoparticles of the lipid nanoparticle composition have a surface fluidity value of greater than a threshold polarization level.

### Ionizable Lipid

As used herein, the term “ionizable lipid” has its ordinary meaning in the art and may refer to a lipid comprising one or more charged moieties. In some embodiments, an ionizable lipid may be positively charged or negatively charged. For instance, an ionizable lipid may be positively charged at lower pHs, in which case it could be referred to as “cationic lipid.” In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the LNP comprises about 30 mol % to about 60 mol %, about 35 mol % to about 55 mol %, about 40 mol % to about 50 mol %, or about 45 mol % to about 50 mol % of ionizable lipid.

In some embodiments, the ionizable lipid is an ionizable amino lipid. In one embodiment, the ionizable amino lipid may have a positively charged hydrophilic head and a hydrophobic tail that are connected via a linker structure.

In some embodiments, the ionizable lipid is a compound of Formula (I):

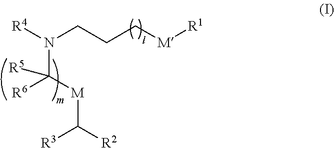

or an N-oxide or a salt thereof, wherein:

R¹ is

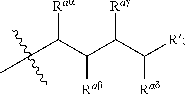

wherein

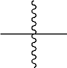

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

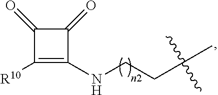

wherein n is selected from 1, 2, 3, 4, and 5;
wherein

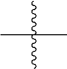

denotes a point of attachment,
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I), or an N-oxide or a salt thereof, wherein:
R¹ is

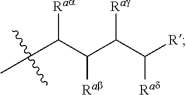

wherein

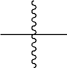

denotes a point of attachment;
R^{aα}, R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

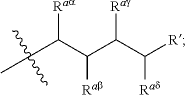

wherein

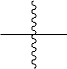

denotes a point of attachment;
R^{aα}, R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 3; and
m is 7.

In some embodiments, the ionizable lipid is a compound of Formula (I), or an N-oxide or a salt thereof, wherein:
R¹ is

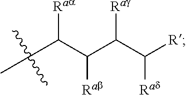

wherein

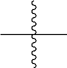

denotes a point of attachment;
R^{aα}, is C₂₋₁₂ alkyl;
R^{aβ}, R^{aγ}, and R^{aδ} are each H;
R² and R³ are each C₁₋₁₄ alkyl;
R⁴ is

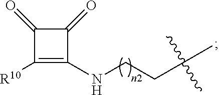

R¹⁰ is —NH(C₁₋₆ alkyl);
n2 is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

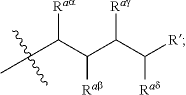

wherein

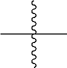

denotes a point of attachment;
R^{aα}, R^{aβ}, and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is selected from:

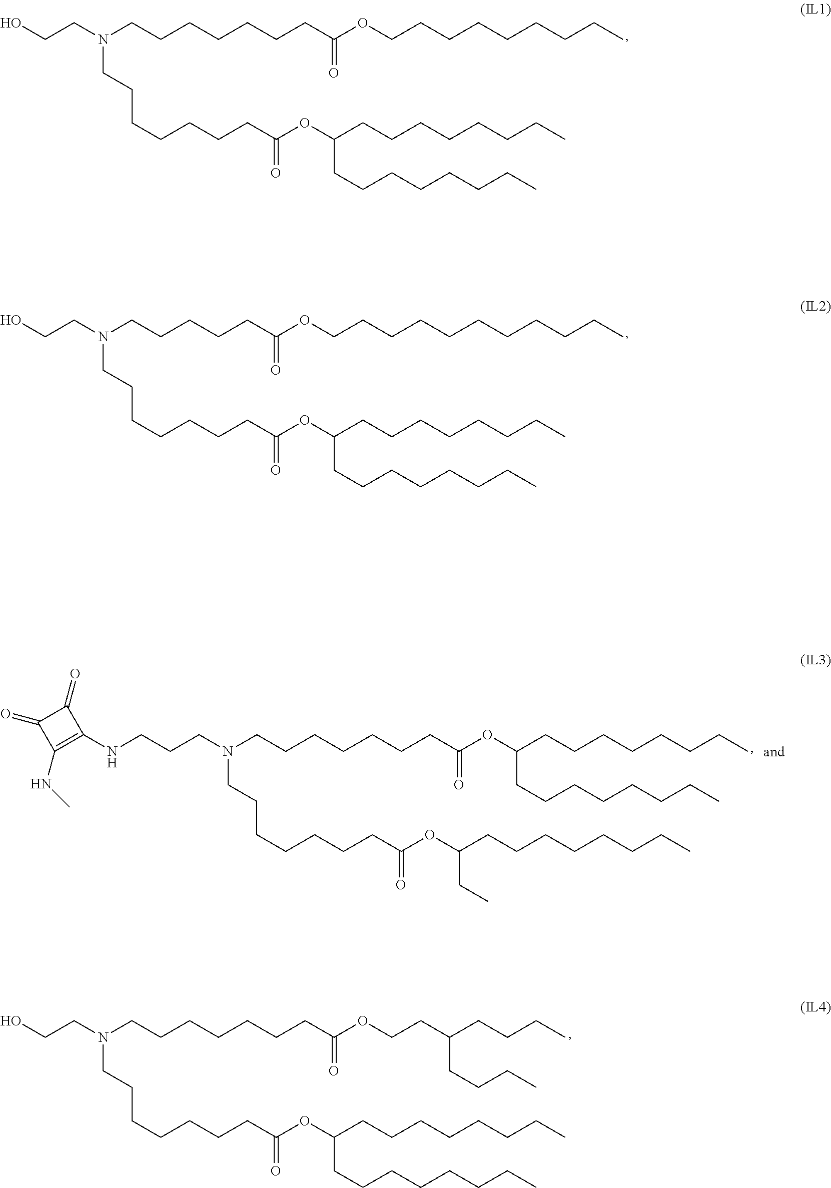

or an N-oxide or a salt thereof.

In some embodiments, the ionizable lipid is the compound:

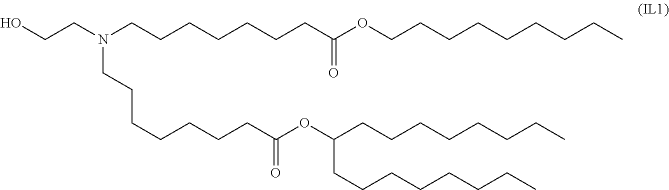 [SMILES: CCCCCCCCCOC(=O)CCCCCCCN(CCO)CCCCCCCC(=O)OC(CCCCCCCC)CCCCCCCC]

or an N-oxide or a salt thereof.

In some embodiments, the ionizable lipid is the compound:

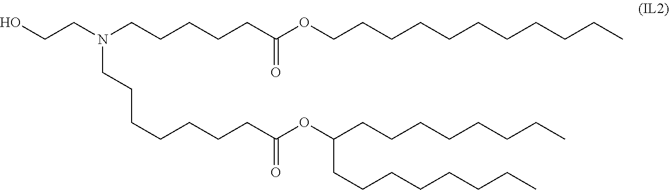 [SMILES: CCCCCCCCCCCOC(=O)CCCCCN(CCO)CCCCCCCC(=O)OC(CCCCCCCC)CCCCCCCC]

or an N-oxide or a salt thereof.

In some embodiments, the ionizable lipid is the compound:

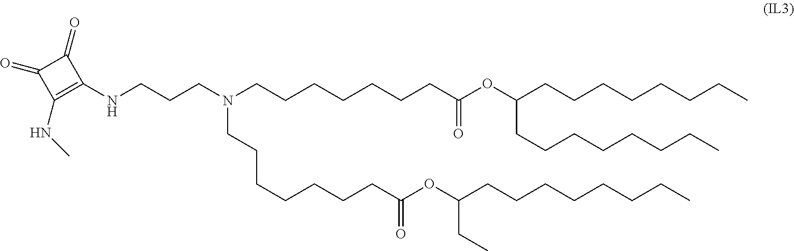 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is the compound:

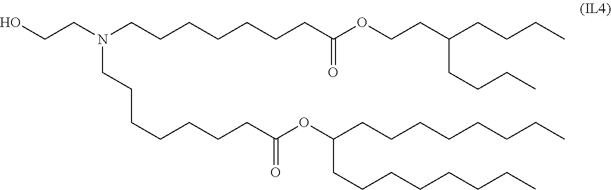 [SMILES: CCCCCCCCC(CCCCCCCC)OC(=O)CCCCCCCN(CCO)CCCCCCCC(=O)OCCC(CCCC)CCCC]

or an N-oxide or a salt thereof.

In some embodiments, the ionizable lipid is a compound of Formula (I):

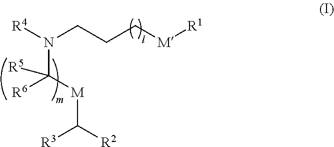

or an N-oxide or a salt thereof, wherein:
R¹ is:

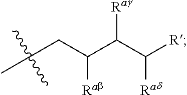

wherein

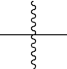

denotes a point of attachment;
R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and

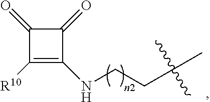

wherein

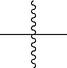

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I):

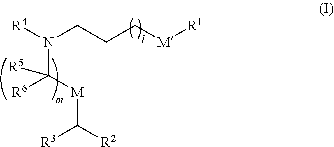

or an N-oxide or a salt thereof, wherein:
R¹ is

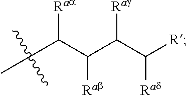

wherein

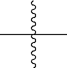

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is —(CH₂)ₙOH, wherein n is selected from 1, 2, 3, 4, and 5;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments, the ionizable lipid is a compound of Formula (I), or an N-oxide or a salt thereof, wherein:
R¹ is

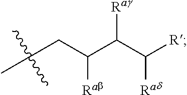

wherein

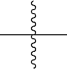

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I), or an N-oxide or a salt thereof, wherein:
R¹ is

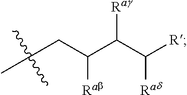

wherein

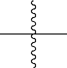

denotes a point of attachment;
R^{aβ}, R^{aγ}, and R^{aδ} are each H;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 3; and
m is 7.

In some embodiments, the ionizable lipid is a compound of Formula (I), or an N-oxide or a salt thereof, wherein:
R¹ is

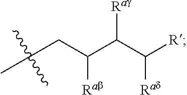

wherein

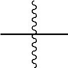

denotes a point of attachment;
R^{aβ} and R^{aδ} are each H;
R^{aγ} is C₂₋₁₂ alkyl;
R² and R³ are each C₁₋₁₄ alkyl;
R⁴ is —(CH₂)ₙOH;
n is 2;
each R⁵ is H;
each R⁶ is H;
M and M′ are each —C(O)O—;
R′ is C₁₋₁₂ alkyl;
l is 5; and
m is 7.

In some embodiments, the ionizable lipid is a compound of Formula (I):

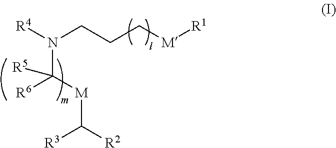

or an N-oxide or a salt thereof, wherein:
R¹ is

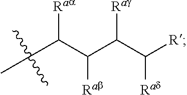

wherein

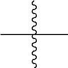

denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is

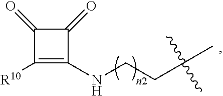

wherein

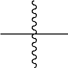

denotes a point of attachment;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

In some embodiments:
R¹ is

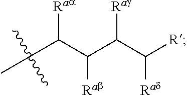

wherein

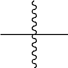

denotes a point of attachment;
R^{aβ}, R^{aγ}, and R^{aδ} are each H;
R^{aα} is C₂₋₁₂ alkyl;
R² and R³ are each C₁₋₁₄ alkyl;
R⁴ is

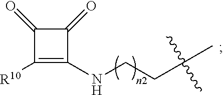

wherein

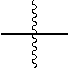

denotes a point of attachment;
wherein R¹⁰ is NH(C₁₋₆ alkyl);
wherein n2 is 2;
each R⁵ is H;
each R⁶ is H;
M and M′ are each —C(O)O—;
R′ is C₁₋₁₂ alkyl;
l is 5; and
m is 7.

In some embodiments, the ionizable lipid of Formula (I) is:

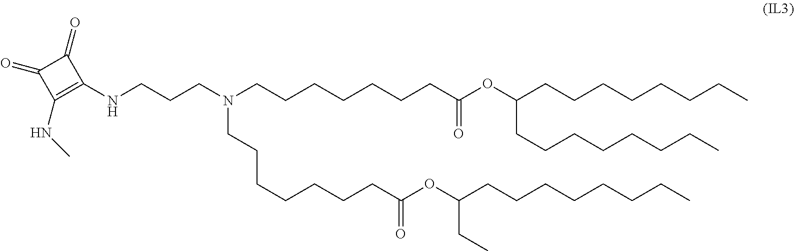 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

or an N-oxide or a salt thereof.

In some embodiments, the ionizable lipid is a compound of Formula (II):

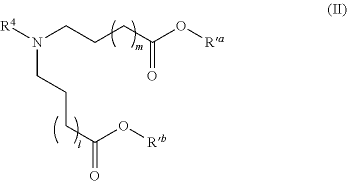

or an N-oxide or a salt thereof, wherein:
R′^{a} is R′^{branched} or R′^{cyclic}; wherein
R′^{branched} is:

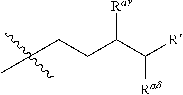

### and R′^{cyclic} is:

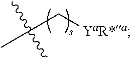

and
R′^{b} is:

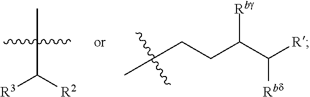

wherein

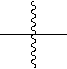

denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from the C₁₋₁₄ alkyl and C₂₋₁₄ alkenyl;
R⁴ is selected from —(CH₂)ₙOH and

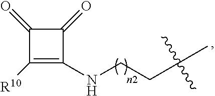

wherein

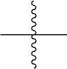

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
Y^{a} is a C₃₋₆ carbocycle;
R*″^{a} is selected from C₁₋₁₅ alkyl and C₂ alkenyl;
s is 2 or 3;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

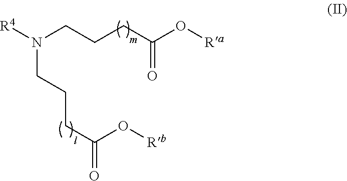

or an N-oxide or a salt thereof, wherein:
R′^{a} is R′^{branched} or R′^{cyclic}; wherein
R′^{branched} is:

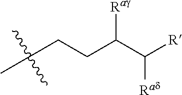

### and R′^{b} is:

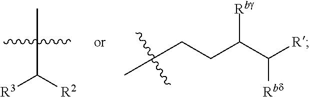

wherein

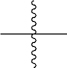

denotes a point of attachment;
R^{aγ} and R^{aδ} are each independently selected from H, C₁₋₁₂ alkyl, and C₂₋₁₂ alkenyl, wherein at least one of R^{aγ} and R^{aδ} is selected from C₁₋₁₂ alkyl and C₂₋₁₂ alkenyl;
R^{bγ} and R^{bδ} are each independently selected from H, C₁₋₁₂ alkyl, and C₂₋₁₂ alkenyl, wherein at least one of R^{bγ} and R^{bδ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

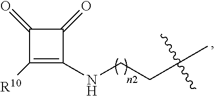

wherein

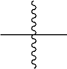

denotes a point of attachment;
wherein n is selected from of 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

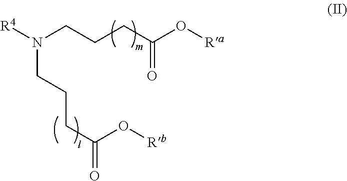

or an N-oxide or a salt thereof, wherein:
R′^{a} is R′^{branched} or R′^{cyclic}; wherein
R′^{branched} is:

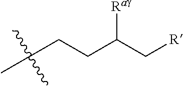

### and R′^{b} is:

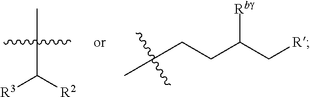

wherein

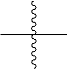

denotes a point of attachment;
R^{aγ} and R^{bγ} are each independently selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

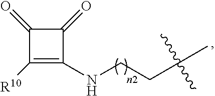

wherein

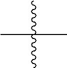

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H; and
wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

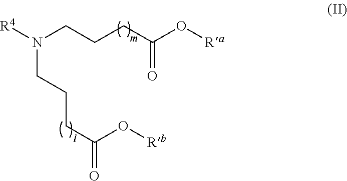

or an N-oxide or a salt thereof, wherein:
R′^{a} is R′^{branched} or R′^{cyclic};
R′^{branched} is:

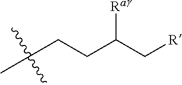

### and R′^{b} is:

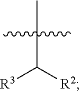

wherein

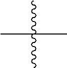

denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is selected from —(CH₂)ₙOH and

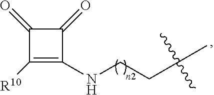

wherein

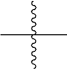

denotes a point of attachment;
wherein n is selected from 1, 2, 3, 4, and 5;
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
R′ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

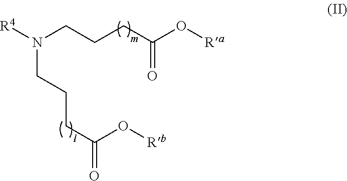

or an N-oxide or a salt thereof, wherein:
R′^{a} is R′^{branched} or R′^{cyclic};
R′^{branched} is:

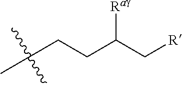

### and R′^{b} is:

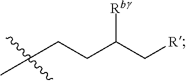

wherein

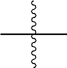

denotes a point of attachment;
R^{aγ} and R^{bγ} are each independently selected from C₁₋₁₂ alkyl and C₂₋₁₂ alkenyl;
R⁴ is selected from —(CH₂)ₙOH and

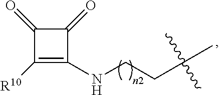

wherein

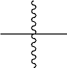

a denotes a point of attachment;
wherein n is selected from 1, 2, 3, 4, and 5;
wherein R¹⁰ d is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R′ independently is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, the ionizable lipid is a compound of Formula (II):

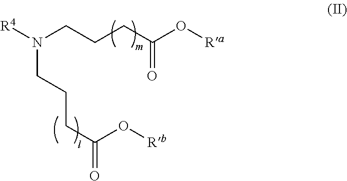

or an N-oxide or a salt thereof, wherein:
R′^{a} is R′^{branched} or R′^{cyclic}; wherein
R′^{branched} is:

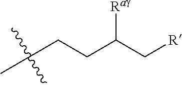

### R and R′^{b} is:

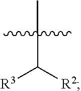

wherein

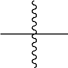

denotes a point of attachment;
R^{aγ} is selected from C₁₋₁₂ alkyl and C₂₋₁₂ alkenyl;
R² and R³ are each independently selected from C₁₋₁₄ alkyl and C₂₋₁₄ alkenyl;
R⁴ is —(CH₂)ₙOH wherein n is selected from 1, 2, 3, 4, and 5;
R′ is C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments, m and l are each independently selected from 4, 5, and 6. In some embodiments m and l are each 5.

In some embodiments each R′ independently is C₁₋₁₂ alkyl. In some embodiments, each R′ independently is C₂₋₅ alkyl.

In some embodiments, R′^{b} is:

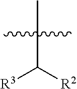

and R² and R³ are each independently C₁₋₁₄ alkyl.

In some embodiments, R′^{b} is:

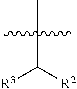

and R² and R³ are each independently C₆₋₁₀ alkyl.

In some embodiments, R′^{b} is:

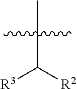

and R² and R³ are each C₈ alkyl.

In some embodiments, R′^{branched} is:

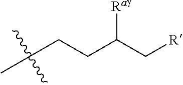

### and R′^{b} is:

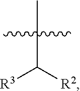

R^{aγ} is C₁₋₁₂ alkyl and R² and R³ are each independently C₆₋₁₀ alkyl.

In some embodiments, R′^{branched} is:

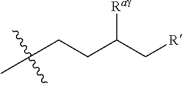

### and R′^{b} is:

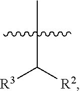

R^{aγ} is a C₂₋₆ alkyl and R² and R³ are each independently C₆₋₁₀ alkyl. In some embodiments, R′^{branched} is:

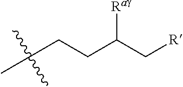

### and R′^{b} is:

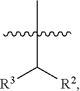

R^{aγ} is C₂₋₆ alkyl, and R² and R³ are each a C₈ alkyl.

In some embodiments, R′^{branched} is:

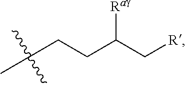

### R′^{b} is:

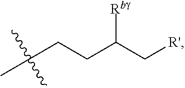

and R^{aγ} and R^{bγ} are each C₁₋₁₂ alkyl.

In some embodiments, R′^{branched} is:

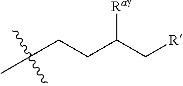

### R′^{b} is:

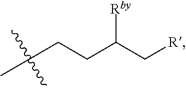

and R^{aγ} and R^{bγ} are each a C₂₋₆ alkyl.

In some embodiments, m and l are each independently selected from 4, 5, and 6 and each R′ independently is C₁₋₁₂ alkyl. In some embodiments, m and l are each 5 and each R′ independently is C₂₋₅ alkyl.

In some embodiments, R′^{branched} is:

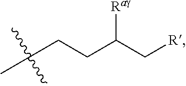

### R′^{b} is:

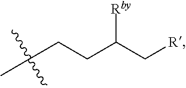

m and l are each independently selected from 4, 5, and 6, each R′ independently is C₁₋₁₂ alkyl, and R^{aγ} and R^{bγ} are each C₁₋₁₂ alkyl.

In some embodiments, R′^{branched} is:

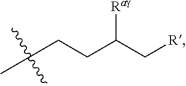

### R′^{b} is:

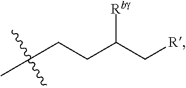

m and l are each 5, each R′ independently is a C₂₋₅ alkyl, and R^{aγ} and R^{bγ} are each a C₂₋₆ alkyl.

In some embodiments, R′^{branched} is:

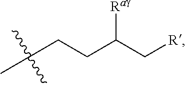

### and R′^{b} is:

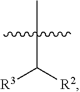

m and l are each independently selected from 4, 5, and 6, R′ is C₁₋₁₂ alkyl, R^{aγ} is C₁₋₁₂ alkyl and R² and R³ are each independently a C₆₋₁₀ alkyl.

In some embodiments, R′^{branched} is:

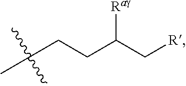

### and R′ is:

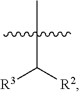

m and l are each 5, R′ is a C₂₋₅ alkyl, R^{aγ} is a C₂₋₆ alkyl, and R² and R³ are each a C₈ alkyl.

In some embodiments, R⁴ is

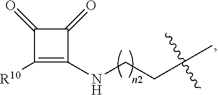

wherein R¹⁰ is NH(C₁₋₆ alkyl) and n2 is 2.

In some embodiments, R⁴ is

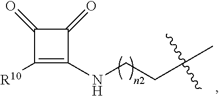

Wherein R¹⁰ is NH(CH₃) and n2 is 2.

In some embodiments, R′^{branched} is:

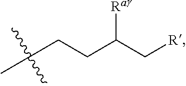

### R′^{b} is:

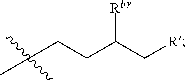

m and l are each independently selected from 4, 5, and 6; each R′ independently is C₁₋₁₂ alkyl; R^{aγ} and R^{bγ} are each C₁₋₁₂ alkyl; and R⁴ is

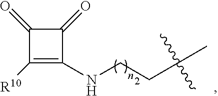

wherein R¹⁰ is NH(C₁₋₆ alkyl), and n2 is 2.

In some embodiments, R′^{branched} is:

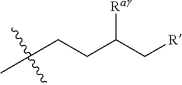

### R′_{b} is:

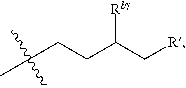

m and l are each 5, each R′ independently is a C₂₋₅ alkyl, R^{aγ} and R^{bγ} are each a C₂₋₆ alkyl, and R⁴ is

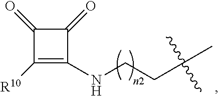

wherein R¹⁰ is NH(CH₃) and n2 is 2.

In some embodiments, R′^{branched} is:

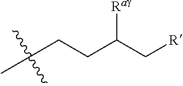

### and R′^{b} is:

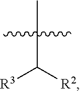

m and l are each independently selected from 4, 5, and 6, R′ is C₁₋₁₂ alkyl, R² and R³ are each independently a C₆₋₁₀ alkyl, R^{aγ} is C₁₋₁₂ alkyl, and R⁴ is

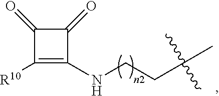

wherein R¹⁰ is NH(C₁₋₆ alkyl) and n2 is 2.

In some embodiments, R′^{branched} is:

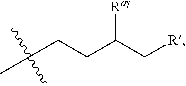

### and R′^{b} is:

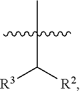

m and 1 are each 5, R′ is a C₂₋₅ alkyl, R^{aγ} is a C₂₋₆ alkyl, R² and R³ are each a C₈ alkyl, and R⁴ is

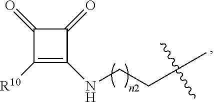

wherein R¹⁰ is NH(CH₃) and n2 is 2.

In some embodiments, R⁴ is —(CH₂)ₙOH and n is 2, 3, or 4. In some embodiments, R⁴ is —(CH₂)ₙOH and n is 2.

In some embodiments, R′^{branched} is:

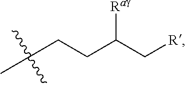

### R′^{b} is:

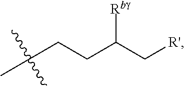

m and l are each independently selected from 4, 5, and 6, each R′ independently is C₁₋₁₂ alkyl, R^{aγ} and R^{bγ} are each C₁₋₁₂ alkyl, R⁴ is —(CH₂)ₙOH, and n is 2, 3, or 4.

In some embodiments, R′^{branhed} is:

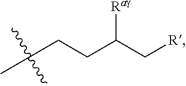

### R′^{b} is:

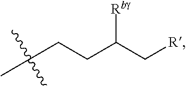

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable lipid is a compound of Formula (II):

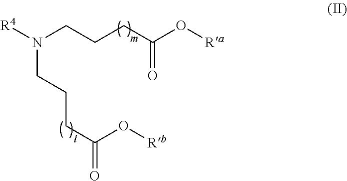

or an N-oxide or a salt thereof, wherein:
R′^{a} is R′^{branched} or R′^{cyclic}; wherein
R′^{branched} is:

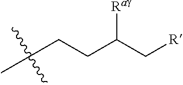

### and R′^{b} is:

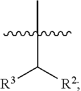

wherein

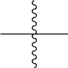

denotes a point of attachment;
R^{aγ} is C₁₋₁₂ alkyl;
R² and R³ are each independently C₁₋₁₄ alkyl;
R⁴ is —(CH₂)ₙOH wherein n is selected from 1, 2, 3, 4, and 5;
R′ is C₁₋₁₂ alkyl;
m is selected from 4, 5, and 6; and
l is selected from 4, 5, and 6.

In some embodiments, m and l are each 5, and n is 2, 3, or 4.

In some embodiments, R′ is a C₂₋₅ alkyl, R^{aγ} is a C₂₋₆ alkyl, and R² and R³ are each C₆₋₁₀ alkyl.

In some embodiments, m and l are each 5, n is 2, 3, or 4, R′ is a C₂₋₅ alkyl, R^{aγ} is C₂₋₆ alkyl, and R² and R³ are each a C₆₋₁₀ alkyl.

In some embodiments, the ionizable lipid is a compound of Formula (II-g):

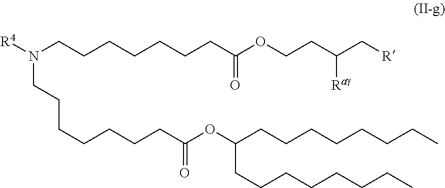

or an N-oxide or salt thereof, wherein:
R^{aγ} is C₂₋₆ alkyl;
R′ is C₂₋₅ alkyl; and
R⁴ is selected from —(CH₂)ₙOH and

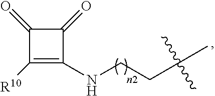

wherein

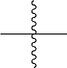

denotes a point of attachment,
wherein n is selected from 3, 4, and 5; and
wherein R¹⁰ is NH(C₁₋₆ alkyl); and
wherein n2 is selected from 1, 2, and 3.

In some embodiments, the ionizable lipid is a compound of Formula (II-h):

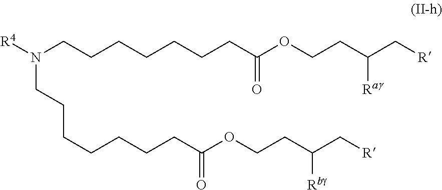

or an N-oxide or salt thereof, wherein:
R^{aγ} and R^{bγ} are each independently a C₂₋₆ alkyl;
each R′ independently is a C₂₋₅ alkyl; and
R⁴ is selected from —(CH₂)ₙOH and

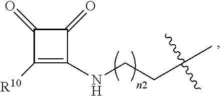

wherein

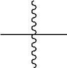

denotes a point of attachment,
wherein n is selected from 3, 4, and 5;
wherein R¹⁰ is NH(C₁₋₆ alkyl); and
wherein and n2 is selected from 1, 2, and 3.

In some embodiments, R⁴ is

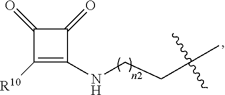

wherein R¹⁰ is NH(CH₃) and n2 is 2.

In some embodiments, R⁴ is —(CH₂)₂OH.

In some embodiments, the ionizable lipid is a compound having Formula (III):

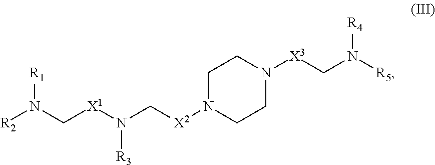

or an N-oxide or a salt thereof, wherein:
R₁, R₂, R₃, R₄, and R₅ are independently selected from C₅₋₂₀ alkyl, C₅₋₂₀ alkenyl, —R″MR′, —R*YR″, —YR″, and —R*OR″;
each M is independently selected
from —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are each independently selected from a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from C₁₋₁₂ alkyl and C₂₋₁₂ alkenyl;
each R is independently selected from C₁₋₃ alkyl and a C₃₋₆ carbocycle;
each R′ is independently selected from C₁₋₁₂ alkyl, C₂₋₁₂ alkenyl, and H; and
each R″ is independently selected from C₃₋₁₂ alkyl and C₃₋₁₂ alkenyl, and wherein:
   i) at least one of X¹, X², and X³ is not —CH₂—; and/or
   ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each —C(O)—.

In some embodiments, the compound of Formula (III) is:

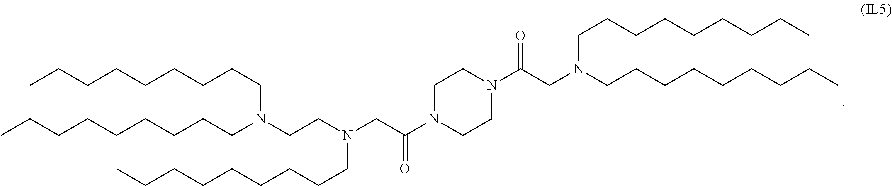 [SMILES: CCCCCCCCCN(CCCCCCCCC)CCN(CCCCCCCCC)CC(=O)N1CCN(C(=O)CN(CCCCCCCCC)CCCCCCCCC)CC1]

### Phospholipid

Phospholipids, as defined herein, are any lipids that comprise a phosphate group. Phospholipids are a subset of non-cationic lipids. The LNP core may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some embodiments, the LNP comprises about 5 mol % to about 15 mol %, about 8 mol % to about 13 mol %, or about 10 mol % to about 12 mol % of phospholipid.

In some embodiments, the phospholipid is a compound of Formula (IV):

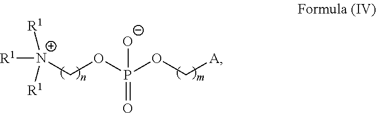

or a salt thereof, wherein:
each R¹ is independently H or optionally substituted alkyl; or optionally two R¹ are joined together with the intervening atoms to form optionally substituted monocyclic cycloalkyl or optionally substituted monocyclic heterocyclyl; or optionally three R¹ are joined together with the intervening atoms to form optionally substituted bicyclic cycloalkyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;

A is of the formula:

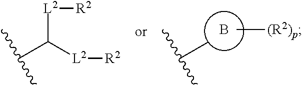

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)_{O}—, —OS(O)_{O}—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2;
provided that the compound is not of the formula:

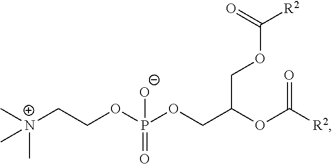

wherein each instance of R² is independently unsubstituted alkyl, unsubstituted alkenyl, or unsubstituted alkynyl.

In some embodiments, the phospholipids is selected from:
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-diphytanoyl-sn-glycero-3-phosphocholine (4ME 16:0 PC),
1,2-diphytanoyl-sn-glycero-3-phospho-(1′-rac-glycerol) (sodium salt) (4ME 16:0 PG),
1,2-diphytanoyl-sn-glycero-3-phospho-L-serine (sodium salt) (4ME 16:0 PS),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), and Sphingomyelin.

In some embodiments, the phospholipid is DSPC, DOPE, or combinations thereof. In some embodiments, the phospholipid is DSPC. In some embodiments, the phospholipid is DOPE. In some embodiments, the phospholipid is 4ME 16:0 PE, 4ME 16:0 PC, 4ME 16:0 PG, 4ME 16:0 PS, or combination thereof.

In some embodiments, the phospholipid is N-lauroyl-D-erythro-sphinganylphosphorylcholine.

### Alternative Lipids

In certain embodiments, an alternative lipid is used in place of a phospholipid. Non-limiting examples of such alternative lipids include the following:

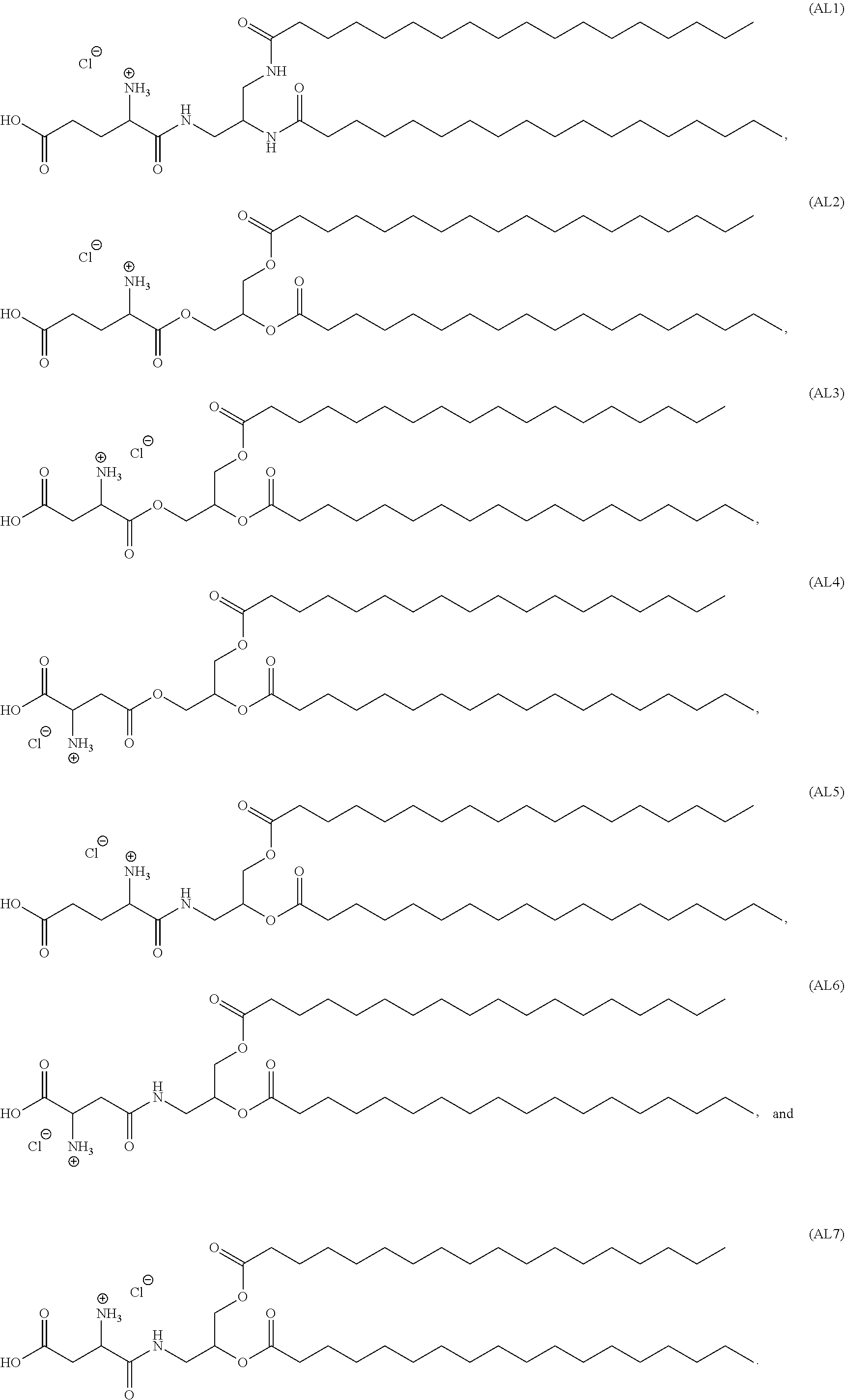

### Structural Lipid

The LNP core may include one or more structural lipids. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols.

In some embodiments, the structural lipid is alpha-tocopherol. In certain embodiments, the structural lipid is a steroid. In certain embodiments, the structural lipid is cholesterol. In certain embodiments, the structural lipid is an analog of cholesterol. In some embodiments, the structural lipid is β-sitosterol. In certain embodiments, the structural lipid is cholesteryl hemisuccinate. Cholesteryl hemisuccinate has the following structure:

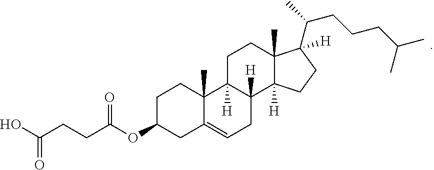

In some embodiments, the LNP comprises about 20 mol % to about 60 mol %, about 30 mol % to about 50 mol %, or about 35 mol % to about 40 mol % of structural lipid. In some embodiments, the LNP comprises about 35 mol % of structural lipid. In some embodiments, the LNP comprises about 40 mol % structural lipid.

### PEG and PEG-Modified Lipids

The LNP core may include one or more molecules comprising polyethylene glycol (PEG), such PEG-modified lipids. Such species may be alternately referred to as PEGylated lipids. The PEG-lipid is a lipid modified with polyethylene glycol. The PEG-lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof.

In some embodiments, the PEG lipid is a compound of Formula (V):

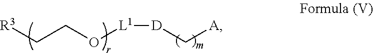

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted cycloalkylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;

A is of the formula:

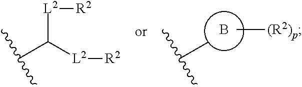

each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2.

In some embodiments, the PEG lipid is PEG-c-DOMG, PEG-DMG, PEG-DLPE, PEG-DMPE, PEG-DPPC, or PEG-DSPE. In some embodiments, the PEG lipid is PEG-DMG. In some embodiments, the PEG lipid is PEG-DMG 2k. In some embodiments, a PEG lipid has the structure:

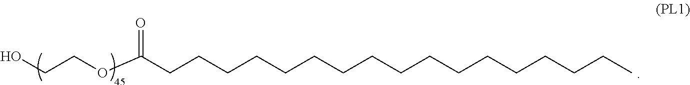

DMG-PEG 2k has the following structure:

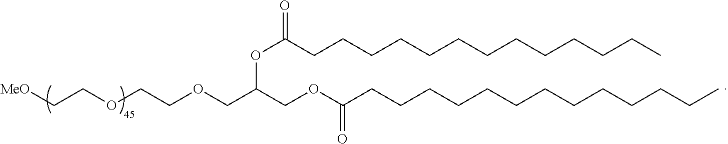

In some embodiments, the PEG-modified lipid is a modified form of PEG-DMG.

In one embodiment, PEG-lipids can be PEGylated lipids described in International Publication No. WO2012099755, the contents of which is herein incorporated by reference in its entirety.

Any of the PEG-lipids described herein may be modified to comprise one or more hydroxyl group on the PEG chain (OH-PEG-lipids) or one or more hydroxyl group on the lipid (PEG-lipid-OH). In some embodiments, the PEG-lipid is an OH-PEG-lipid. In some embodiments, the OH-PEG-lipid comprises a hydroxyl group at the terminus of the PEG chain. In some embodiments, the PEG-lipids described herein may be modified to comprise one or more alkyl group on the PEG chain (alkyl-PEG-lipid). In some embodiments, the alkyl-PEG-lipid is a methoxy-PEG-lipid.

In some embodiments, the LNP comprises about 0.1 mol % to about 5.0 mol %, about 0.5 mol % to about 5.0 mol %, about 1.0 mol % to about 5.0 mol %, about 1.0 mol % to about 2.5 mol %, about 0.5 mol % to about 2.0 mol %, or about 1.0 mol % to about 1.5 mol % of PEG-lipid. In some embodiments, the LNP comprises about 1.5 mol % or about 3.0 mol % PEG-lipid.

Certain of the LNPs provided herein comprise no or low levels of PEG-lipid. Some LNPs comprise less than 0.5 mol % PEG-lipid.

In some embodiments, PEG is used as a stabilizer. In some embodiments, the PEG stabilizer is a PEG-lipid. In some embodiments, the LNP comprises less than 0.5 mol % PEG stabilizer.

### Payload Molecules

The lipid nanoparticle compositions of the disclosure can be used to deliver a wide variety of different payloads to cells. The payload can be a therapeutic or prophylactic agent capable of mediating (e.g., directly mediating or via a bystander effect) a therapeutic or prophylactic effect in such cell. Typically the payload delivered by the composition is a nucleic acid, although non-nucleic acid agents, such as small molecules, chemotherapy drugs, peptides, polypeptides and other biological molecules are also encompassed by the disclosure. Nucleic acids that can be delivered include DNA-based molecules (i.e., comprising deoxyribonucleotides) and RNA-based molecules (i.e., comprising ribonuleotides). Furthermore, the nucleic acid can be a naturally occurring form of the molecule or a chemically-modified form of the molecule (e.g., comprising one or more modified nucleotides).

In one embodiment, the therapeutic or prophylactic is an agent that enhances (i.e., increases, stimulates, upregulates) protein expression. Non-limiting examples of types of therapeutic or prophylactic agents that can be used for enhancing protein expression include RNAs, mRNAs, dsRNAs, CRISPR/Cas9 technology, ssDNAs and DNAs (e.g., expression vectors).

In one embodiment, the therapeutic or prophylactic agent is an agent that reduces (i.e., decreases, inhibits, downregulates) protein expression. Non-limiting examples of types of therapeutic or prophylactic that can be used for reducing protein expression include mRNAs that incorporate a micro-RNA binding site(s) (miR binding site), microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNAs), locked nucleic acids (LNAs) and CRISPR/Cas9 technology.

In one embodiment, the therapeutic or prophylactic is a peptide therapeutic agent. In one embodiment the therapeutic or prophylactic a polypeptide therapeutic agent. In some embodiments, the therapeutic or prophylactic agent comprises an mRNA encoding: a secreted protein; a membrane-bound protein; or an intercellular protein, or peptides, polypeptides or biologically active fragments thereof.

In some embodiments, at least about 50%, at least about 75%, at least about 90%, or at least about 95% of the payload is encapsulated within the lipid nanoparticle. In some embodiments, about 50% to about 99%, about 65% to about 99%, about 75% to about 95%, or about 80% to about 95% of the payload is encapsulated within the lipid nanoparticle.

### Cells

The LNPs can be used to deliver payload molecules to a population of cells. In some embodiments, the LNPs are contacted with a population of cells. In some embodiments, about 10% or greater, 15% or greater, 20% or greater, or 30% or greater of the cell population has accumulated the LNPs when the LNPs are contacted with the cell population. In some embodiments, about 1% to about 75%, about 5% to about 50%, about 10% to about 40%, or about 15% to about 25% of the cell population has accumulated the LNPs when the LNPs are contacted with the cell population.

In some embodiments, about 5% or greater, about 10% or greater, or about 20% or greater of cells in the population of cells expresses the payload when the LNP is contacted with the population of cells. In some embodiments, about 0.5% to about 50%, about 1% to about 40%, about 3% to about 20%, or about 5% to about 15% of cells in the population of cells expresses the payload when the LNP is contacted with the population of cells.

In some embodiments, the cell population is an epithelial cell population. In some embodiments, the cell population is a respiratory epithelial cell population. In some embodiments, the respiratory epithelial cell population is a lung cell population. In some embodiments, the respiratory epithelial cell population is a nasal cell population. In some embodiments, the respiratory epithelial cell population is an alveolar epithelial cell population. In some embodiments, the respiratory epithelial cell population is a bronchial epithelial cell population. In some embodiments, the respiratory epithelial cell population is an HBE population. In some embodiments, the cell population is a HeLa population.

### Pharmaceutical Compositions and Formulations

The present disclosure provides pharmaceutical compositions and formulations that comprise any of LNPs described herein.

Pharmaceutical compositions or formulations can optionally comprise one or more additional active substances, e.g., therapeutically and/or prophylactically active substances. Pharmaceutical compositions or formulations of the present disclosure can be sterile and/or pyrogen-free. General considerations in the formulation and/or manufacture of pharmaceutical agents can be found, for example, in Remington: The Science and Practice of Pharmacy 21^{st} ed., Lippincott Williams & Wilkins, 2005 (incorporated herein by reference in its entirety). In some embodiments, compositions are administered to humans, human patients or subjects. For the purposes of the present disclosure, the phrase “active ingredient” generally refers to the nanoparticle comprising the polynucleotides or polypeptide payload to be delivered as described herein.

Formulations and pharmaceutical compositions described herein can be prepared by any method known or hereafter developed in the art of pharmacology. In general, such preparatory methods include the step of associating the nanoparticle with an excipient and/or one or more other accessory ingredients, and then, if necessary and/or desirable, dividing, shaping and/or packaging the product into a desired single- or multi-dose unit.

A pharmaceutical composition or formulation in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

A pharmaceutically acceptable excipient, as used herein, includes, but is not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cryoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (povidone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA formulations. In order to prevent oxidation, antioxidants can be added to the formulations. Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisole, ethylenediamine, sodium lauryl sulfate (SLS), sodium lauryl ether sulfate (SLES), etc., and combinations thereof.

In some embodiments, the pH of polynucleotide solutions are maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition described here can contain a bulking agent in lyophilized polynucleotide formulations to yield a “pharmaceutically elegant” cake, stabilize the lyophilized polynucleotides during long term (e.g., 36 month) storage. Exemplary bulking agents of the present disclosure can include, but are not limited to sucrose, trehalose, mannitol, glycine, lactose, raffinose, and combinations thereof.

The compositions can be in a liquid form or a solid form. In some embodiments, the compositions or formulations are in a liquid form. In some embodiments, the compositions are suitable for inhalation. The compositions can be administered to the pulmonary tract. Aerosolized pharmaceutical formulations can be delivered to the lungs, preferably using a number of commercially available devices.

Compositions can be administered to the respiratory tract by suitable methods such as intranasal instillation, intratracheal instillation, and intratracheal injection. In some embodiments, the compositions or the nanoparticle is administered by intranasal, intrabronchial, or pulmonary administration. For example, the compositions and nanoparticles are administered by nebulizer or inhaler.

In some embodiments, the compositions are delivered into the lungs by inhalation of an aerosolized pharmaceutical formulation. Inhalation can occur through the nose and/or the mouth of the subject. Administration can occur by self-administration of the formulation while inhaling, or by administration of the formulation via a respirator to a subject on a respirator. Exemplary devices for delivering formulations to the lung include, but are not limited to, dry powder inhalers, pressurized metered dose inhalers, nebulizers, and electrohydrodynamic aerosol devices.

Liquid formulations can be administered to the lungs of a patient using a pressurized metered dose inhaler (pMDI). pMDIs generally include at least two components: a canister in which the liquid formulation is held under pressure in combination with one or more propellants, and a receptacle used to hold and actuate the canister. The canister may contain single or multiple doses of the formulation. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the liquid formulation is atomized, forming an aerosol. pMDIs typically employ one or more propellants to pressurize the contents of the canister and to propel the liquid formulation out of the receptacle outlet, forming an aerosol. Any suitable propellants may be utilized. The propellant may take a variety of forms. For example, the propellant may be a compressed gas or a liquefied gas.

The liquid formulations can also be administered using a nebulizer. Nebulizers are liquid aerosol generators that convert the liquid formulation into mists or clouds of small droplets, preferably having diameters less than 5 microns mass median aerodynamic diameter, which can be inhaled into the lower respiratory tract. This process is called atomization. The droplets carry the one or more active agents into the nose, upper airways or deep lungs when the aerosol cloud is inhaled. Any type of nebulizer may be used to administer the formulation to a patient, including, but not limited to pneumatic (jet) nebulizers and electromechanical nebulizers. Pneumatic (jet) nebulizers use a pressurized gas supply as a driving force for atomization of the liquid formulation. Compressed gas is delivered through a nozzle or jet to create a low pressure field which entrains a surrounding liquid formulation and shears it into a thin film or filaments. The film or filaments are unstable and break up into small droplets that are carried by the compressed gas flow into the inspiratory breath. Baffles inserted into the droplet plume screen out the larger droplets and return them to the bulk liquid reservoir. Electromechanical nebulizers use electrically generated mechanical force to atomize liquid formulations. The electromechanical driving force can be applied, for example, by vibrating the liquid formulation at ultrasonic frequencies, or by forcing the bulk liquid through small holes in a thin film. The forces generate thin liquid films or filament streams which break up into small droplets to form a slow-moving aerosol stream which can be entrained in an inspiratory flow. Liquid formulations can also be administered using an electrohydrodynamic (EHD) aerosol device. EHD aerosol devices use electrical energy to aerosolize liquid drug solutions or suspensions.

Dry powder inhalers (DPIs) typically use a mechanism such as a burst of gas to create a cloud of dry powder inside a container, which can then be inhaled by the subject. In a DPI, the dose to be administered is stored in the form of a non-pressurized dry powder and, on actuation of the inhaler, the particles of the powder are inhaled by the subject. In some cases, a compressed gas (i.e., propellant) may be used to dispense the powder, similar to pressurized metered dose inhalers (pMDIs). In some cases, the DPI may be breath actuated, meaning that an aerosol is created in precise response to inspiration. Typically, dry powder inhalers administer a dose of less than a few tens of milligrams per inhalation to avoid provocation of cough. Examples of DPIs include the Turbohaler® inhaler (Astrazeneca, Wilmington, Del.), the Clickhaler® inhaler (Innovata, Ruddington, Nottingham, UKL), the Diskus® inhaler (Glaxo, Greenford, Middlesex, UK), the EasyHaler® (Orion, Expoo, FI), the Exubera® inhaler (Pfizer, New York, N.Y.), the Qdose® inhaler (Microdose, Monmouth Junction, N.J.), and the Spiros® inhaler (Dura, San Diego, Calif.).

The pharmaceutical compositions are administered in an effective amount to cause a desired biological effect, e.g., a therapeutic or prophylactic effect, e.g., owing to expression of a normal gene product to supplement or replace a defective protein or to reduce expression of an undesired protein, as measured by, in some embodiments, the alleviation of one or more symptoms. The formulations may be administered in an effective amount to deliver LNP to, e.g., the apical membrane of respiratory and non-respiratory epithelial cells to deliver a payload.

### Methods of Use

The lipid amine compounds can be used to prepare lipid nanoparticle compositions which can be loaded with a payload and administered to cells, such as cells in patients for the treatment of disease. Accordingly, provided herein are methods of delivering a payload into a cell such as by contacting the cell with a lipid nanoparticle composition disclosed herein.

In some embodiments, the cell is an epithelial cell. In some embodiments, the cell is an airway epithelial cell. In some embodiments, the cell is a respiratory epithelial cell. The respiratory epithelial cell can be, for example, a lung cell, a nasal cell, an alveolar epithelial cell, or a bronchial epithelial cell. In some embodiments, the cell is an HBE cell or a HeLa population. In some embodiments, the cell is in a patient.

In some embodiments, the payload is a polynucleotide or polypeptide. The polynucleotide include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a βD-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof. In some embodiments, the polynucleotide is mRNA, rRNA, or tRNA. In some embodiments, the polynucleotide is mRNA.

The lipid nanoparticle composition can be administered to patient by intranasal, intrabronchial, or pulmonary administration. For example, the compositions and nanoparticles can be administered by nebulizer or inhaler.

As will be appreciated by those skilled in the art, the lipid amines disclosed herein have additional uses. For example, lipid amines can be used to treat inflammatory diseases. Lipid amines can also be used as antimicrobial agents.

### Kits and Devices

The present disclosure provides a variety of kits for conveniently and/or effectively using the claimed nanoparticles of the present disclosure. Typically, kits will comprise sufficient amounts and/or numbers of components to allow a user to perform multiple treatments of a subject(s) and/or to perform multiple experiments.

In one aspect, the present disclosure provides kits comprising the nanoparticles of the present disclosure.

The kit can further comprise packaging and instructions and/or a delivery agent to form a formulation composition. The delivery agent can comprise a saline, a buffered solution, a lipidoid or any delivery agent disclosed herein. In one embodiment, such a kit further comprises an administration device such as a nebulizer or an inhaler.

### Process of Preparing LNPs

The present disclosure also provides a process of preparing a lipid nanoparticle composition comprising contacting a lipid nanoparticle core disclosed herein with a lipid amine disclosed herein.

In some embodiments, a process of preparing a lipid nanoparticle composition comprises:
(a) mixing a nucleic acid payload with a lipid solution comprising:
   (1) an ionizable lipid,
   (2) a phospholipid,
   (3) a structural lipid, and
   (4) optionally a PEG-lipid resulting in a filled lipid nanoparticle (fLNP) core; and
(c) contacting the fLNP core with the lipid amine.

In some embodiments, a process of preparing a nanoparticle comprises:

(a) mixing a lipid solution comprising:
   (1) an ionizable lipid,
   (2) a phospholipid,
   (3) a structural lipid, and
   (4) optionally a PEG-lipid
      resulting in an empty lipid nanoparticle (eLNP) core;
(b) contacting the eLNP core with a nucleic acid payload forming an fLNP; and
(c) contacting the fLNP core with the lipid amine.

In some embodiments, the mixing further comprises an aqueous buffer solution. In some embodiments, the aqueous buffer solution has a pH of about 3.5 to about 4.5. In further embodiments, the aqueous buffer solution has a pH of about 4. In some embodiments, the aqueous buffer solution has a pH of about 4.6 to about 6.5. In some embodiments, the aqueous buffer solution has a pH of about 5.

In some embodiments, the aqueous buffer solution can comprise an acetate buffer, a citrate buffer, a phosphate buffer, or a Tris buffer. In some embodiments, the aqueous buffer solution comprises an acetate buffer or a citrate buffer. In further embodiments, the aqueous buffer solution is an acetate buffer, such as a sodium acetate buffer.

In some embodiments, the aqueous buffer solution has a buffer concentration greater than about 30 mM. In some embodiments, the aqueous buffer solution has a buffer concentration greater than about 40 mM. In some embodiments, the aqueous buffer solution has a buffer concentration of about 30 mM to about 100 mM. In some embodiments, the aqueous buffer solution has a buffer concentration of about 40 mM to about 75 mM. In further embodiments, the aqueous buffer solution has a buffer concentration of about 33 mM, about 37.5 mM, or about 45 mM.

In some embodiments, the aqueous buffer solution can have an ionic strength of about 15 mM or less, about 10 mM or less, or about 5 mM or less. In some embodiments, the aqueous buffer solution has an ionic strength of about 0.1 mM to about 15 mM, about 0.1 mM to about 10 mM, or about 0.1 mM to about 5 mM.

In some embodiments, the lipid solution has a lipid concentration of about 5 to about 100 mg/mL, about 15 to about 35 mg/mL, about 20 to about 30 mg/mL, or about 24 mg/mL.

The lipid solution can further comprise an organic solvent such as an alcohol, e.g., ethanol. The organic solvent can be present in an amount of about 1% to about 50%, about 5% to about 40%, or about 10% to about 33% by volume. In further embodiments, the solvent in is 100% ethanol or greater than 95% ethanol by volume.

In some embodiments, the lipid solution comprises about 30 mol % to about 60 mol %, about 35 mol % to about 55 mol %, or about 40 mol % to about 50 mol % of ionizable lipid with respect to total lipids. In some embodiments, the lipid solution comprises about 5 mol % to about 15 mol %, about 8 mol % to about 13 mol %, or about 10 mol % to about 12 mol % of phospholipid with respect to total lipids. In some embodiments, the lipid solution comprises about 30 mol % to about 50 mol %, about 35 mol % to about 45 mol %, or about 37 mol % to about 42 mol % of structural lipid with respect to total lipids. In some embodiments, the lipid solution comprises about 0.1 mol % to about 2 mol %, about 0.1 mol % to about 1 mol %, or about 0.25 mol % to about 0.75 mol % of PEG-lipid with respect to total lipids.

In some embodiments, the lipid solution comprises:
about 40 mol % to about 50 mol % of ionizable lipid;
about 10 mol % to about 12 mol % of phospholipid;
about 37 mol % to about 42 mol % of structural lipid; and
about 0.25 mol % to about 0.75 mol % of PEG-lipid; each with respect to total lipids.

In some embodiments, the lipid solution comprises:
about 49 mol % of ionizable lipid;
about 11 mol % to about 12 mol % of phospholipid;
about 39 mol % of structural lipid; and
about 0.5 mol % of PEG-lipid; each with respect to total lipids.

The mixing of the lipid solution and buffer solution results in precipitation of the lipid nanoparticles and preparation of the herein described empty lipid nanoparticle compositions. Precipitation can be carried out by ethanol-drop precipitation using, for example, high energy mixers (e.g., T-junction, confined impinging jets, microfluidic mixers, vortex mixers) to introduce lipids (in ethanol) to a suitable anti-solvent (i.e. water) in a controllable fashion, driving liquid supersaturation and spontaneous precipitation into lipid particles. In some embodiments, the mixing is carried out with a multi-inlet vortex mixer. In some embodiments, the mixing is carried out with a microfluidic mixer, such as described in WO 2014/172045. The mixing step can be performed at ambient temperature or, for example, at a temperature of less than about 30° C., less than about 28° C., less than about 26° C., less than about 25° C., less than about 24° C., less than about 22° C., or less than about 20° C.

In some embodiments, the mixing comprises nanoprecipitation. Nanoprecipitation is the unit operation in which the nanoparticles are self-assembled from their individual lipid components by way of kinetic mixing and subsequent maturation and continuous dilution. This unit operation includes three individual steps: mixing of the aqueous and organic inputs, maturation of the nanoparticles, and dilution after a controlled residence time. Due to the continuous nature of these steps, they are considered one unit operation. The unit operation includes the continuous inline combination of three liquid streams with one inline maturation step: mixing of the aqueous buffer with lipid stock solution, maturation via controlled residence time, and dilution of the nanoparticles. The nanoprecipitation itself occurs in the scale-appropriate mixer, which is designed to allow continuous, high-energy, combination of the aqueous solution with the lipid stock solution dissolved in ethanol. The aqueous solution and the lipid stock solution both flow simultaneously into the mixing hardware continuously throughout this operation. The ethanol content, which keeps the lipids dissolved, is abruptly reduced and the lipids all precipitate with each other. The particles are thus self-assembled in the mixing chamber. One of the objectives of unit operation is to exchange the solution into a fully aqueous buffer, free of ethanol, and to reach a target concentration of nanoparticle. This can be achieved by first reaching a target processing concentration, then using diafiltration, and then (if necessary) a final concentration step, once the ethanol has been completely removed.

In some embodiments, the lipid nanoparticle core, which is contacted with the lipid amine, comprises the PEG-lipid. In some embodiments, the lipid nanoparticle core, which is contacted with the lipid amine, is substantially free of PEG-lipid. In some embodiments, the PEG-lipid is added to the lipid nanoparticle together with the lipid amine, prior to the contacting with the lipid amine, or after the contacting with the lipid amine. In some embodiments, the PEG-lipid is used as a stabilizer.

In some embodiments, the contacting of step (b) is carried out at a pH of about 3.5 to about 6.5. In some embodiments, the combining is carried out at a pH of about 5. In some embodiments, the pH of the empty lipid nanoparticle composition is adjusted to about 4.5 to about 5.5 prior to combining the empty lipid nanoparticle composition with payload. In some embodiments, the pH of the empty lipid nanoparticle composition is adjusted to about 5 prior to combining the empty lipid nanoparticle composition with payload.

The nucleic acid payload can be provided as a nucleic acid solution comprising (i) a nucleic acid, such as DNA or RNA (e.g., mRNA), and (ii) a buffer capable of maintaining acidic pH, such as a pH of about 3 to about 6, about 4 to about 6, or about 5 to about 6. In some embodiments, the pH of the nucleic acid solution is about 5.

In some embodiments, the buffer of the nucleic acid solution is an acetate buffer, a citrate buffer, a phosphate buffer, or a tris buffer. In some embodiments, the buffer is an acetate buffer or a citrate buffer. In further embodiments, the buffer is an acetate buffer, such as a sodium acetate buffer. The buffer concentration of the nucleic acid solution can be about 5 mM to about 140 mM. In some embodiments, the buffer concentration is about 20 mM to about 100 mM, about 30 mM to about 70 mM, or about 40 mM to about 50 mM. In some embodiments, the buffer concentration is about 42.5 mM.

The nucleic acid solution can include the nucleic acid at a concentration of about 0.05 to about 5.0 mg/mL, 0.05 to about 2.0 mg/mL, about 0.05 to about 1.0 mg/mL, about 0.1 to about 0.5 mg/mL, or about 0.2 to about 0.3 mg/mL. In some embodiments, the nucleic acid concentration is about 0.25 mg/mL.

High energy mixers (e.g., T-junction, confined impinging jets, microfluidic mixers, vortex mixers) can be used for the contacting of step (b). In some embodiments, the combining is carried out with a multi-inlet vortex mixer. In some embodiments, the combining is carried out with a microfluidic mixer, such as described in WO 2014/172045. The combining step can be performed at ambient temperature or, for example, at a temperature of less than about 30° C., less than about 28° C., less than about 26° C., less than about 25° C., less than about 24° C., less than about 22° C., or less than about 20° C.

In some embodiments, the contacting of the LNP core with a lipid amine comprises dissolving the lipid amine in a non-ionic excipient. In some embodiments, the non-ionic excipient is selected from macrogol 15-hydroxystearate (HS 15), 1,2-dimyristoyl-rac-glycero-3-methoxypolyethylene glycol-2000 (PEG-DMG-2K), PL1, polyoxyethylene sorbitan monooleate [TWEEN®80], and d-α-Tocopherol polyethylene glycol succinate (TPGS). In some embodiments, the non-ionic excipient is macrogol 15-hydroxystearate (HS 15).

In some embodiments, the contacting of the lipid nanoparticle core with a lipid amine comprises the lipid amine dissolved in a buffer solution. In some embodiments, the buffer is an acetate buffer, a citrate buffer, a phosphate buffer, or a tris buffer. In some embodiments, the buffer solution is a phosphate buffered saline (PBS). In some embodiments, the buffer solution is a Tris-based buffer. In some embodiments, the buffer solution concentration is about 5 mM to about 100 mM, about 5 mM to about 50 mM, about 10 mM to about 30 mM, or about 20 mM.

In some embodiments, the lipid amine solution has a pH of about 7 to about 8, or about 7.5. In some embodiments, the concentration of the lipid amine solution is about 0.1 to about 50 mg/mL, about 1 to about 30 mg/mL, about 1 to about 10 mg/mL, or about 2 to about 3 mg/mL.

In some embodiments, the lipid nanoparticle composition undergoes maturation via controlled residence time after loading and prior to neutralization. In some embodiments, the residence time is about 5 to about 120 seconds, about 10 to about 90 seconds, about 20 to about 70 seconds, about 30 to about 60 seconds, about 30 seconds, about 45 seconds, or about 60 seconds.

In some embodiments, the lipid nanoparticle composition undergoes maturation via controlled residence time after neutralization and prior to addition of cationic agent. In some embodiments, the residence time is about 1 to about 30 seconds, about 2 to about 20 seconds, about 5 to about 15 seconds, about 7 to about 12 seconds, or about 10 seconds.

In some embodiments, the processes of preparing lipid nanoparticle compositions further comprise one or more additional steps selected from:
diluting the composition with a dilution buffer;
adjusting the pH of the composition;
adding one or more surface-acting agents to the composition;
filtering the composition;
concentrating the composition;
exchanging buffer of the composition;
adding cryoprotectant to the composition; and
adding an osmolality modifier to the composition.

In some embodiments, the processes of preparing lipid nanoparticle compositions can further comprise 1, 2, 3, 4, 5, 6, 7, or all of the above-listed steps. Some steps may be repeated. The steps can be, but need not be, carried out in the order listed. Each of the steps refers to an action relating to the composition that results from the prior enacted step. For example, if the process includes the step of adding one or more surface-acting agents to the composition, then the surface-acting agent is added to the composition resulting from the previous step, where the previous step could be any of the above-listed steps.

In some embodiments, the one or more additional steps is adjusting the pH of the composition to a pH of about 7 to about 8. In some embodiments, the pH is adjusted to a pH of about 7.5.

In some embodiments, the one or more additional steps is adding a further surface-acting agent to the filled lipid nanoparticle (e.g., in addition to the lipid amine). A surface-acting agent may be disposed within a nanoparticle and/or on its surface (e.g., by coating, adsorption, covalent linkage, or other process). Surface-acting agents may include, but are not limited to, PEG derivatives (e.g., PEG-DMG), lipid amines (e.g. sterol amines and related), anionic proteins (e.g., bovine serum albumin), surfactants (e.g., cationic surfactants such as dimethyldioctadecylammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). In some embodiments, the further surface-acting agent is a PEG lipid, such as PEG-DMG. In some embodiments, the further surface-acting agent is provided together with the lipid amine. In some embodiments, the further surface-acting agent is present together with the lipid amine in the lipid amine solution. In some embodiments, the further surface-acting agent is a PEG-lipid having a concentration of about 0.1 to about 50 mg/mL, about 1 to about 10 mg/mL, or about 1 to about 3 mg/mL.

In some embodiments, the one or more additional step is adding an osmolality modifier to the composition. The osmolality modifier can be a salt or a sugar. In some embodiments, the osmolality modifier is a sugar. The sugar can be selected from, but not limited to glucose, fructose, galactose, sucrose, lactose, maltose, and dextrose. In some embodiments, the osmolality modifier is a salt. The salt can be an inorganic salt, e.g., sodium chloride, potassium chloride, calcium chloride, or magnesium chloride. In some embodiments, the inorganic salt is sodium chloride. In some embodiments, the salt is 4-(2-hydroxyethyl)piperazine-1-ethanesulfonic acid sodium salt. The salt can be provided as a salt solution having a salt concentration of about 100 to about 500 mM, about 200 to about 400 mM, about 250 to about 350 mM, or about 300 mM. The pH of the salt solution can be about 7 to about 8. The salt solution can further include a buffer comprising, for example, an acetate buffer, a citrate buffer, a phosphate buffer, or a tris buffer. The buffer concentration can be, for example, about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM.

Cryoprotectant can be added to the filled nanoparticle composition by the addition of an aqueous cryoprotectant solution which can include an aqueous buffer with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 1 to 20 mM about 1 to about 10 mM, or about 5 mM. In some embodiments, the buffer in the cryoprotectant solution comprises an acetate buffer, a citrate buffer, a phosphate buffer, or a tris buffer. In some embodiments, the buffer is an acetate buffer or a citrate buffer. In further embodiments, the buffer is an acetate buffer, such as a sodium acetate. In some embodiments, the pH of the cryoprotectant solution is about 7 to about 8, such as about 7.5. In some embodiments, the cryoprotectant solution comprises about 40% to about 90%, about 50% to about 85%, about 60% to about 80%, or about 70% by weight of sucrose.

In some embodiments, the processes further include the step of diluting the composition with a dilution buffer. The dilution buffer can be an aqueous buffer solution with a buffer concentration of about 0.1 mM to about 100 mM, about 0.5 mM to about 90 mM, about 1.0 mM to about 80 mM, about 2 mM to about 70 mM, about 3 mM to about 60 mM, about 4 mM to about 50 mM, about 5 mM to about 40 mM, about 6 mM to about 30 mM, about 7 mM to about 20 mM, about 8 mM to about 15 mM, or about 9 mM to about 12 mM. In some embodiments, the buffer concentration is about 30 mM to about 75 mM, about 30 mM to about 60 mM, or about 30 mM to about 50 mM. In some embodiments, the dilution buffer comprises an acetate buffer, a citrate buffer, a phosphate buffer, or a tris buffer. In some embodiments, the dilution buffer comprises an acetate buffer or a citrate buffer. In further embodiments, the dilution buffer is an acetate buffer, such as a sodium acetate. In some embodiments, the pH of the dilution buffer is about 3 to about 7, about 3 to about 6, about 3 to about 5, about 4, about 5, about 5.5, or about 6. In some embodiments, the dilution buffer comprises the same buffer as in the aqueous buffer solution used during the combining of the of the empty lipid nanoparticle composition with the nucleic acid solution.

In some embodiments, the processes further include any one or more of the steps of: filtering the composition; concentrating the composition; and exchanging buffer of the composition. The filtration, concentration, and buffer exchange steps can be accomplished with tangential flow filtration (TFF). Residual organic solvent can be removed by the filtration step.

In some embodiments, buffer exchange can change the composition of the filled lipid nanoparticle composition by raising or lowering buffer concentration, changing buffer composition, or changing pH.

In some embodiments, the concentration step can increase the concentration of the filled lipid nanoparticles in the composition.

In some embodiments, the processes of preparing filled lipid nanoparticle compositions further comprise at least the steps of: adjusting the pH of the composition to a pH of about 7 to about 8 (e.g., about pH 7.5); and adding an osmolality modifier (e.g., an inorganic salt) to the composition.

In some embodiments, the processes of preparing filled lipid nanoparticle compositions further comprise at least the steps of: adjusting the pH of the composition to a pH of about 7 to about 8 (e.g., about pH 7.5); adding a surface-acting agent to the composition; and adding an osmolality modifier (e.g., an inorganic salt) to the composition.

In some embodiments, the processes of preparing lipid nanoparticle compositions can further include:
(i) adjusting the pH of the composition to a pH of about 7 to about 8;
(ii) adding one or more surface-acting agents to the composition;
(iii) concentrating the composition;
(iv) adding an inorganic salt to the composition; and
(v) diluting the composition.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the schemes below.

The reactions for preparing compounds described herein can be carried out in suitable solvents, which can be readily selected by one of skill in the art of organic synthesis. Suitable solvents can be substantially non-reactive with the starting materials (reactants), the intermediates, or products at the temperatures at which the reactions are carried out, (e.g., temperatures, which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

Compounds of Formula A1 can be prepared, e.g., using a process as illustrated in the schemes below:

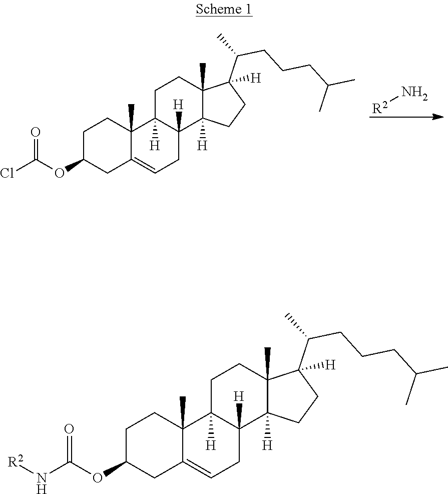

Compounds of Formula A1 can be prepared via the synthetic route outlined in Scheme 1. An appropriate reaction between cholesteryl chloroformate and amines can be carried out under suitable conditions to generate a precursor to a compound of Formula A1 or a compound of Formula A1.

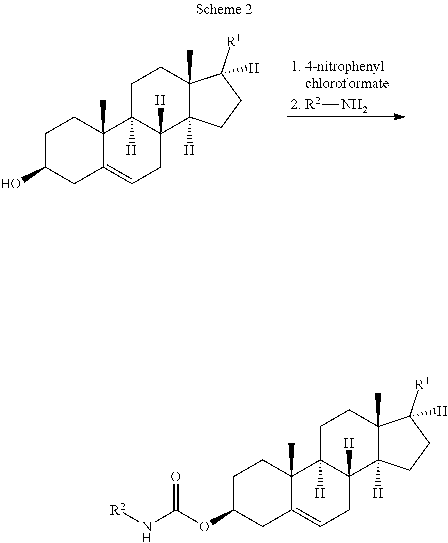

Compounds of Formula A1 can be prepared via the synthetic route outlined in Scheme 2. An appropriate reaction between cholesterol or a cholesterol derivative (such as stigmasterol) and 4-nitrophenyl chloroformate can be carried out under suitable conditions (such as using triethylamine and 4-dimethylaminopyridine). The product of said reaction can be reacted with an amine under suitable conditions (such as using triethylamine) to generate a precursor to a compound of Formula A1 or a compound of Formula A1.

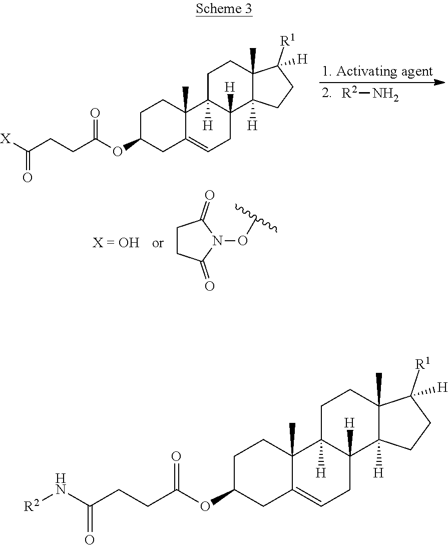

Compounds of Formula A1 can be prepared via the synthetic route outlined in Scheme 3. An appropriate reaction between cholesterol hemisuccinate or a cholesterol derivative hemisuccinate and an activating agent can be carried out under suitable conditions. The product of said reaction can be reacted with an amine under suitable conditions to generate a precursor to a compound of Formula A1 or a compound of Formula A1.

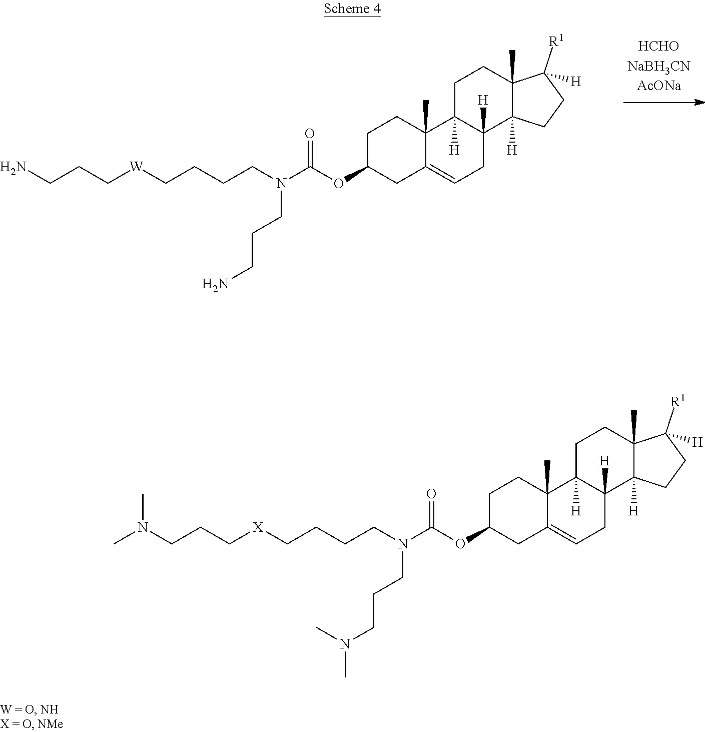

Compounds of Formula A1 can be prepared via the synthetic route outlined in Scheme 4. An appropriate reaction between a compound of Formula A1, HCHO, NaBH₃CN, and AcONa can be carried out under suitable conditions to generate a compound of Formula A1.

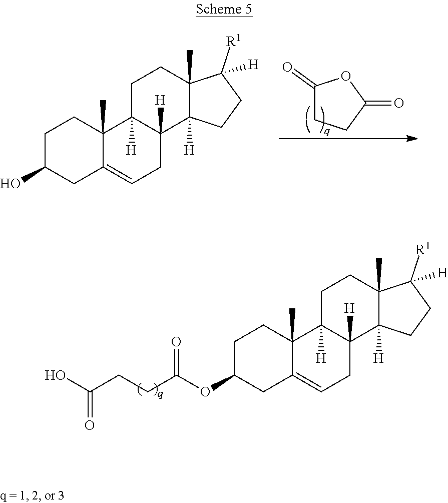

Precursors to compounds of Formula A1 can be prepared via the synthetic route outlined in Scheme 5. An appropriate reaction between cholesterol or a cholesterol derivative (such as stigmasterol) and can be carried out under suitable conditions (such as using triethylamine and 4-dimethylaminopyridine). The product of said reaction can be reacted with an amine under suitable conditions (such as using triethylamine) to give a precursor to a compound of Formula A1.

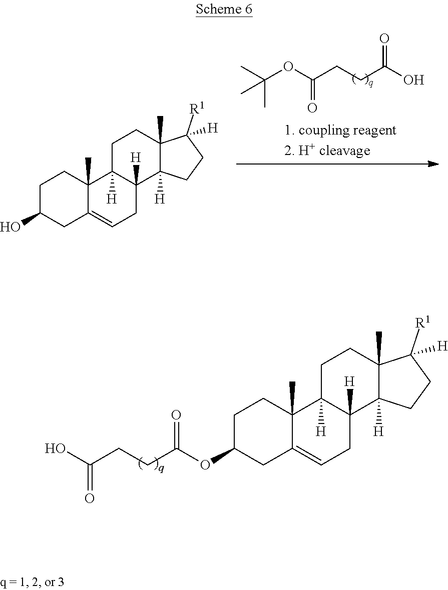

Precursors to compounds of Formula A1 can be prepared via the synthetic route outlined in Scheme 5. An appropriate reaction between cholesterol or a cholesterol derivative (such as stigmasterol) and a boc-hemiester can be carried out under suitable conditions. The product of said reaction can be reacted under suitable conditions to give a precursor to a compound of Formula A1.

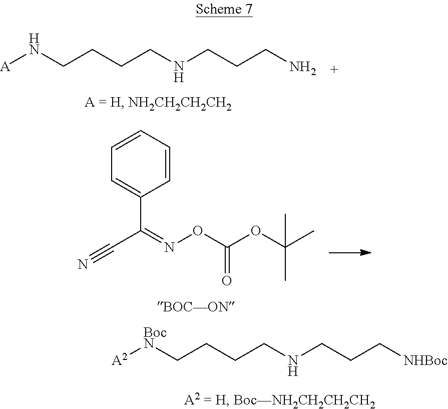

Intermediates for the synthesis of compounds of Formula A1 can be prepared via the synthetic route outlined in Scheme 7. An appropriate reaction between spermidine or spermine and (E)-N-((tert-butoxycarbonyl)oxy)benzimidoyl cyanide (BOC—ON) can be carried out under suitable conditions to give an intermediate for the synthesis of compounds of Formula A1.

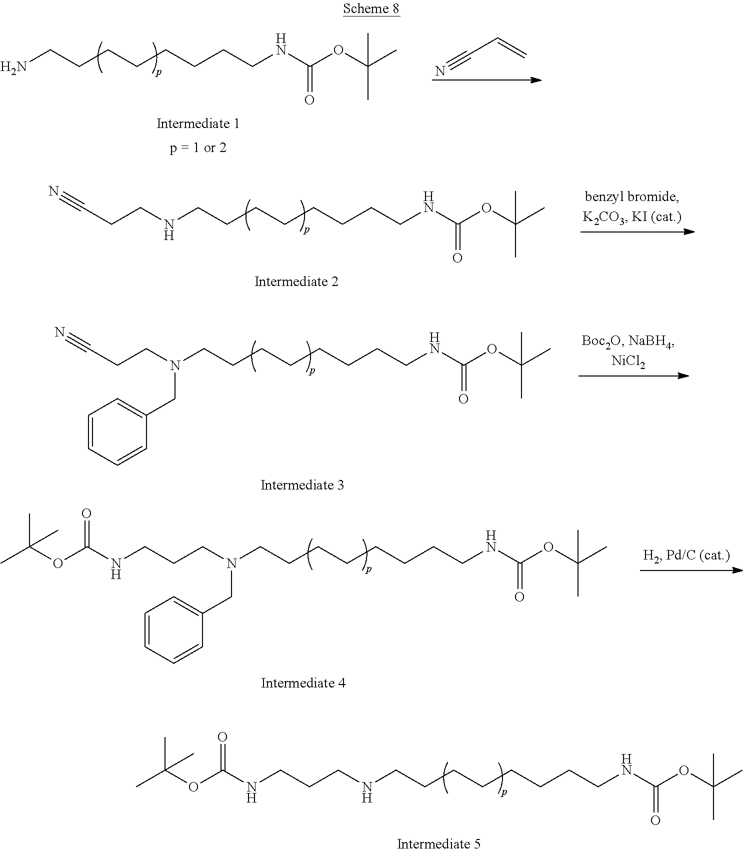

Intermediates for the synthesis of compounds of Formula A1 can be prepared via the synthetic route outlined in Scheme 8. An appropriate reaction between Intermediate 1 and acrylonitrile can be carried out under suitable conditions to give Intermediate 2. Intermediate 2 can be reacted with benzyl bromide under suitable conditions (such as, e.g. K₂CO₃ and KI) to give Intermediate 3. Intermediate 3 can be reacted with Boc₂O under suitable conditions (such as, e.g. NaBH₄ and NiCl₂) to give Intermediate 4. The benzyl group of Intermediate 4 can be removed under suitable conditions (such as H₂ and Pd/C) to give Intermediate 5.

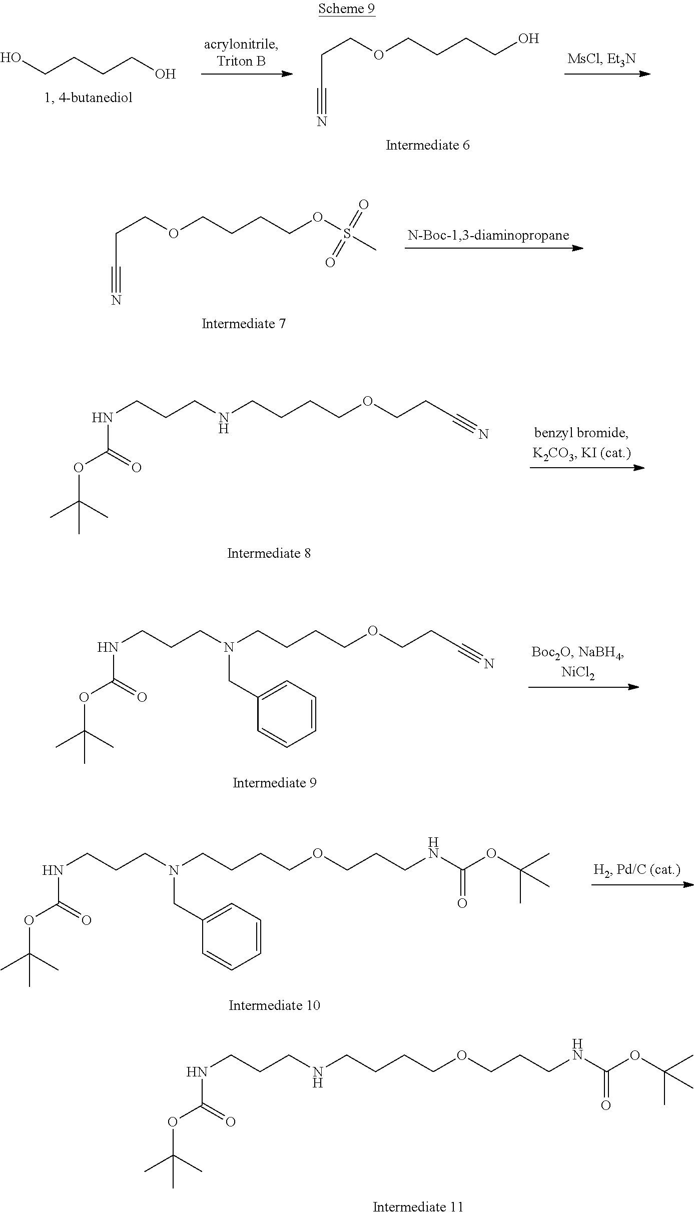

Intermediates for the synthesis of compounds of Formula A1 can be prepared via the synthetic route outlined in Scheme 9. An appropriate reaction between 1,4-butanediol and acrylonitrile can be carried out under suitable conditions (such as, e.g. Triton B) to give Intermediate 6. Intermediate 6 can be reacted with methanesulfonyl chloride under suitable conditions (such as, e.g. triethylamine) to give Intermediate 7. Intermediate 7 can be reacted with N-Boc-1,3-diaminopropane under suitable conditions to give intermediate 8. Intermediate 8 can be reacted with benzyl bromide under suitable conditions (such as, e.g. K₂CO₃ and KI) to give Intermediate 9. Intermediate 9 can be reacted with Boc₂O under suitable conditions (such as, e.g. NaBH₄ and NiCl₂) to give Intermediate 10. The benzyl group of Intermediate 10 can be removed under suitable conditions (such as, e.g. H₂ and Pd/C) to give Intermediate 11.

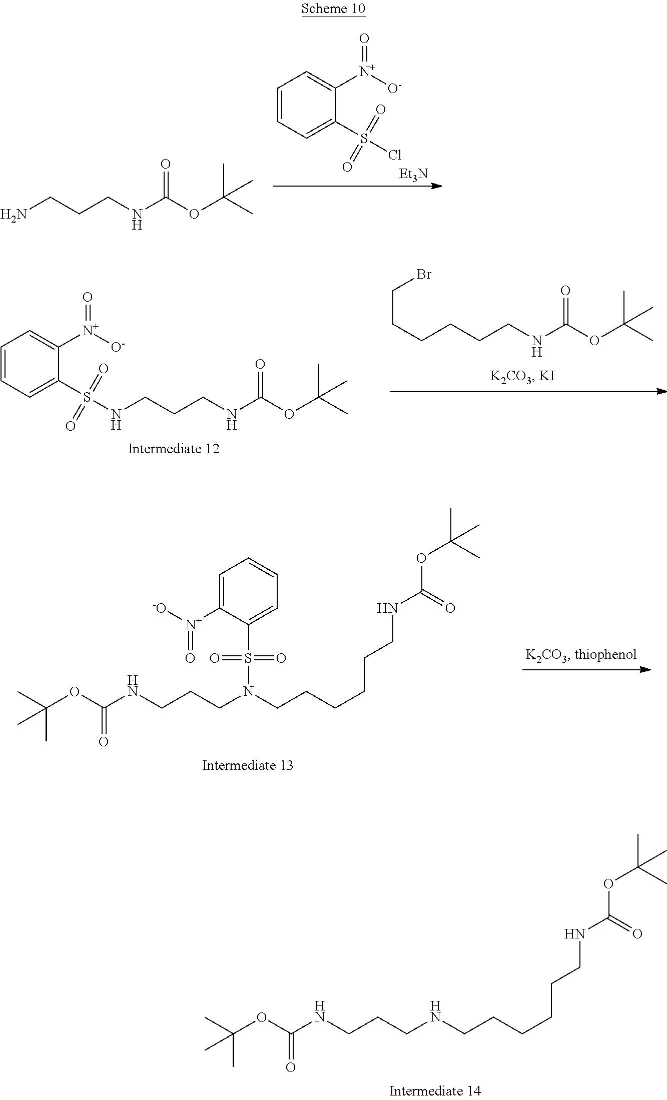

Intermediates for the synthesis of compounds of Formula A1 can be prepared via the synthetic route outlined in Scheme 10. An appropriate reaction between N-Boc-1,3-diaminopropane and 2-nitrobenzenesulfonyl chloride under suitable conditions (such as, e.g. triethylamine) to give Intermediate 12. Intermediate 12 can be reacted with tert-butyl N-(6-bromohexyl)carbamate under suitable conditions (such as, e.g. K₂CO₃ and KI) to give Intermediate 13. The 2-nitrobenzenesulfonyl group can be removed under suitable conditions (such as, e.g. K₂CO₃ and thiophenol) to give Intermediate 14.

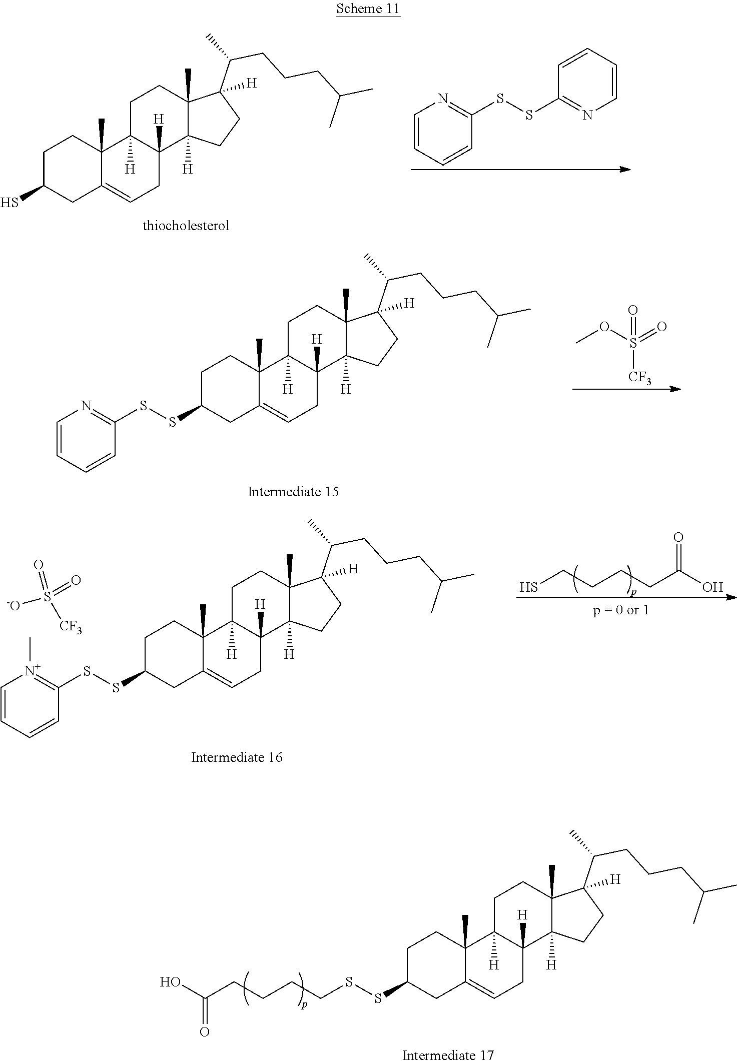

Intermediates for the synthesis of compounds of Formula A1 can be prepared via the synthetic route outlined in Scheme 11. An appropriate reaction between thiocholesterol and 2,2′-dipyridyldisulfide under suitable conditions give Intermediate 15. Intermediate 15 can be reacted with methyl trifluoromethanesulfonate (methyl triflate) under suitable conditions to give Intermediate 16. Intermediate 16 can be reacted with an appropriate mercaptocarboxylic acid to afford Intermediate 17.

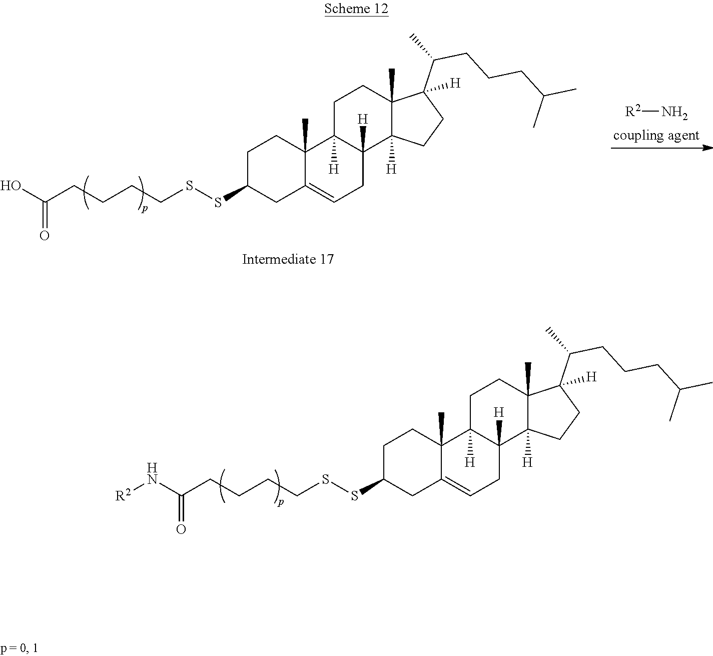

Compounds of Formula A1 can be prepared via the synthetic route outlined in Scheme 12. An appropriate reaction between Intermediate 17 and an amine can be carried out under suitable conditions (such as using a coupling agent) to generate a precursor to a compound of Formula A1 or a compound of Formula A1.

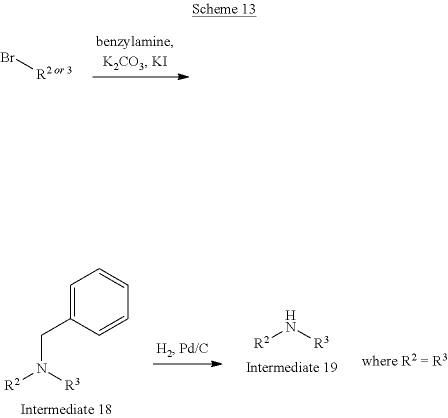

Intermediates for the synthesis of compounds of Formula A1 can be prepared via the synthetic route outlined in Scheme 13. An appropriate reaction between benzylamine and an alkyl halide under suitable conditions (such as, e.g. K₂CO₃ and KI) gives Intermediate 18. The benzyl group of Intermediate 18 is removed under suitable conditions (such as, e.g. H₂ and Pd/C) to give Intermediate 19.

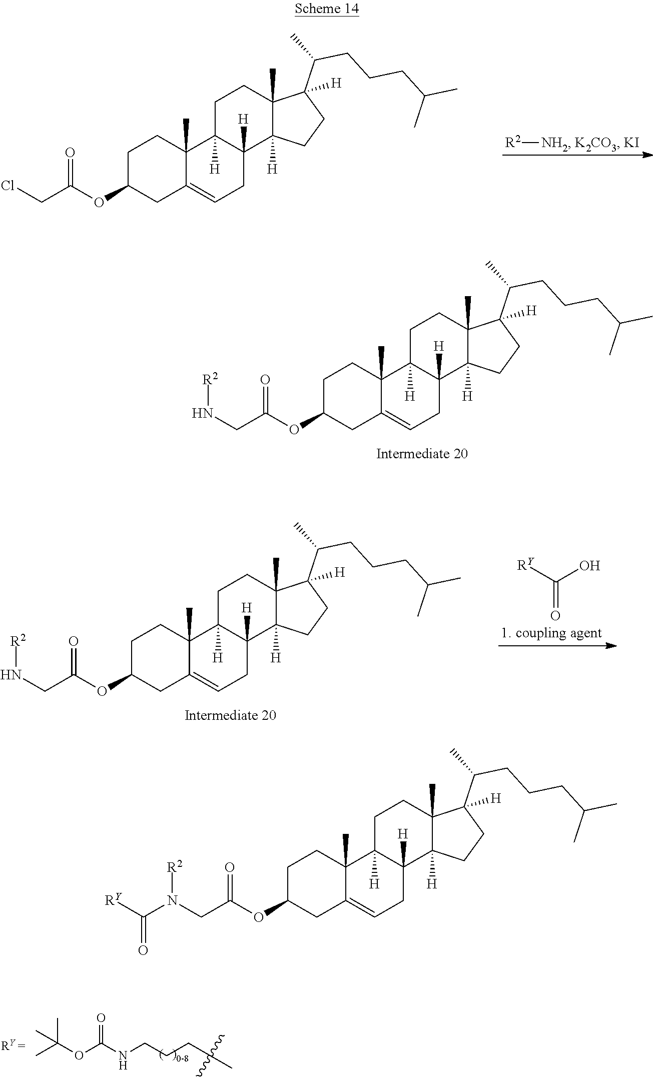

Compounds of Formula A1 or precursors for the synthesis of compounds of Formula A1 can be prepared via the synthetic route outlined in Scheme 14. An appropriate reaction between cholesterol chloroacetate and an amine under suitable conditions (such using, e.g. K₂CO₃ and KI) to give Intermediate 20. Intermediate 20 can be reacted with an appropriate carboxylic acid under suitable conditions to generate a precursor compound of Formula A1 or a compound of Formula A1. In some embodiments, R^{Y} is

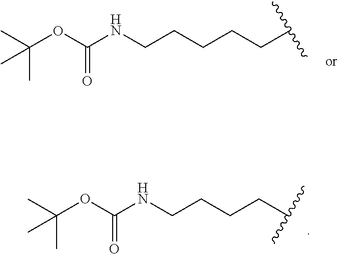 [SMILES: *CCCCCNC(=O)OC(C)(C)C ; *CCCCNC(=O)OC(C)(C)C]

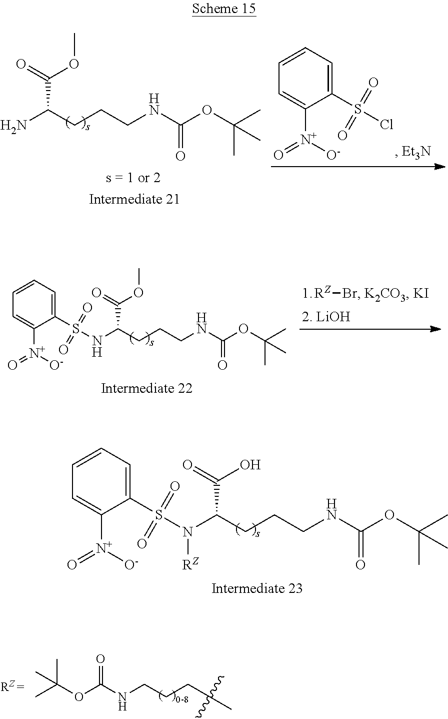

Precursors to compounds of Formula A1 can be prepared via the synthetic route outlined in Scheme 15. An appropriate between Intermediate 21 and nosyl chloride can be carried out under suitable conditions (such as, e.g., triethylamine) to give Intermediate 22. Intermediate 22 can be reacted with an alkyl bromide under suitable conditions (such as, e.g., K₂CO₃ and KI) to give Intermediate 23. In some embodiments, Rz is

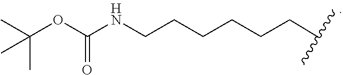 [SMILES: *CCCCCCNC(=O)OC(C)(C)C]

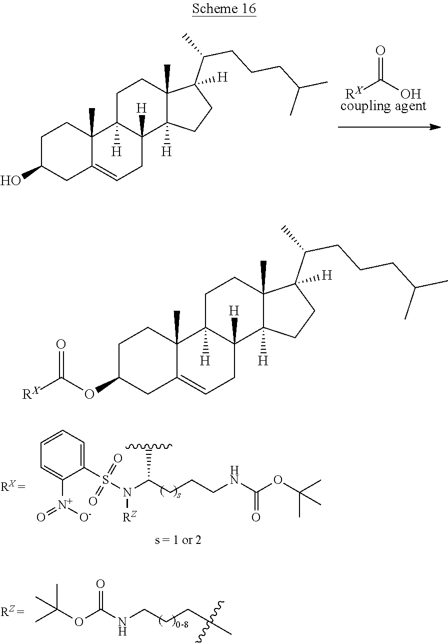

Precursors to compounds of Formula A1 can be prepared via the synthetic route outlined in Scheme 16. An appropriate reaction between cholesterol and a carboxylic acid can be carried out under suitable conditions in the presence of a coupling agent. The product of said reaction can be reacted under suitable conditions to give a compound of Formula A1 or a precursor of a compound of Formula A1. In some embodiments, Rx is

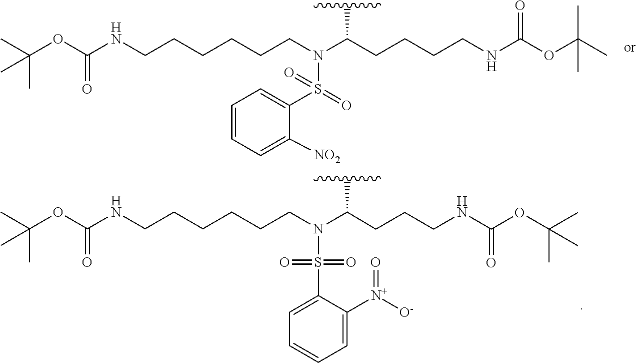

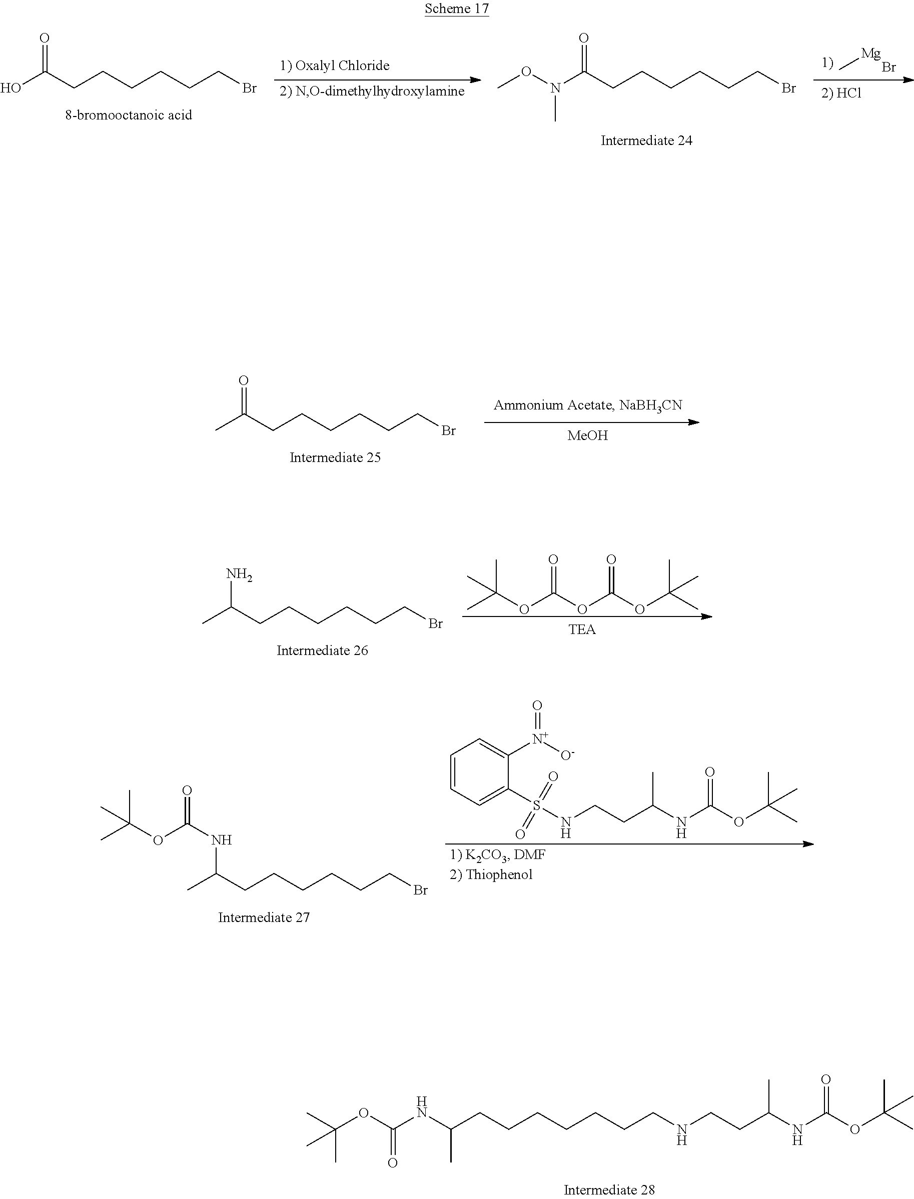

Intermediates for the synthesis of compounds of Formula A1 can be prepared via the synthetic route outlined in Scheme 17. An appropriate reaction between 8-bromooctanoic acid, oxalyl chloride, and N,O-dimethylhydroxlamine can be carried out under suitable conditions (such as, e.g. catalytic DMF in DCM) to give Intermediate 24. Intermediate 24 can be reacted with methylmagnesium bromide and HCl under suitable conditions to give Intermediate 25. Intermediate 25 can be reacted with ammonium acetate and sodium cyanoborohydride under suitable conditions to give Intermediate 26. Intermediate 26 can be reacted with BOC-anhydride under suitable conditions (such as, e.g. triethylamine (TEA)) to give Intermediate 27. Intermediate 27 can be reacted with tert-butyl (4-((2-nitrophenyl)sulfonamido)butan-2-yl)carbamate (prepared similarly to Intermediate 12) under suitable conditions (such as, e.g. K₂CO₃, BnBr, and thiophenol) to give Intermediate 28.

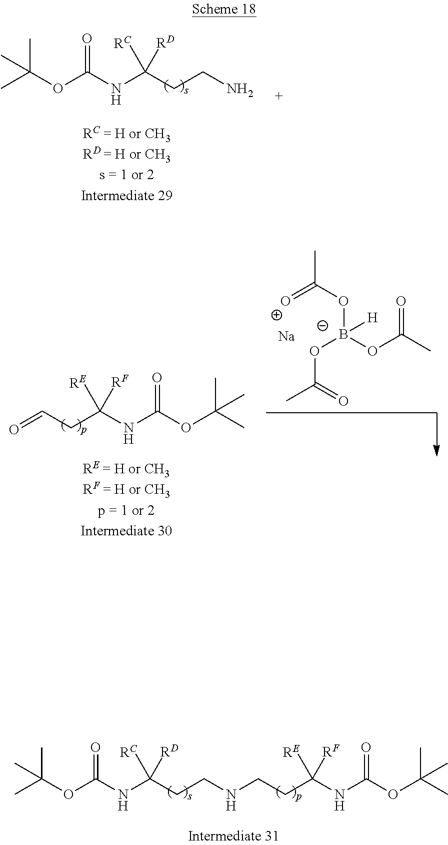

Intermediates for the synthesis of compounds of Formula A1 can be prepared via the synthetic route outlined in Scheme 18. An appropriate reaction between Intermediates 29 and 30 under suitable conditions (such as, e.g. sodium triacetoxyborohydride in dry MeOH) to give Intermediate 31.

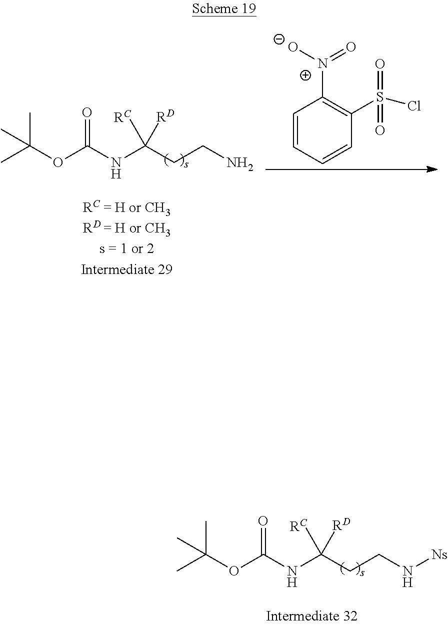

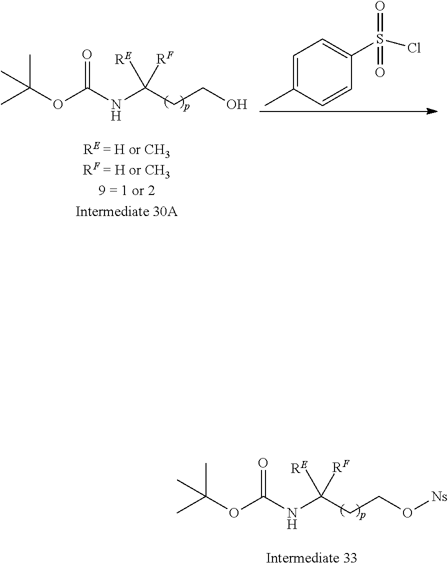

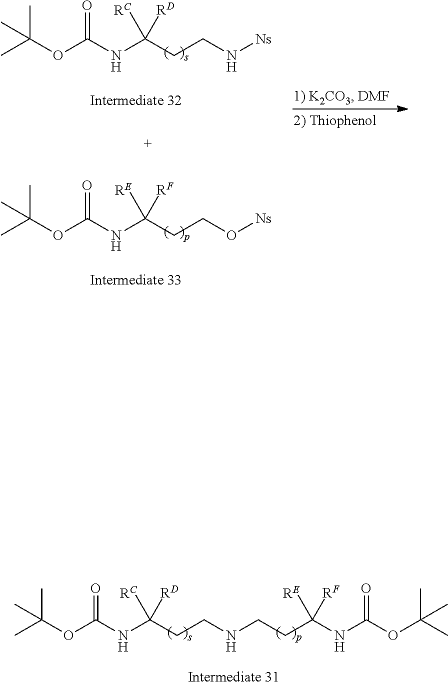

Intermediates for the synthesis of compounds of Formula A1 can be prepared via the synthetic route outlined in Scheme 19. An appropriate reaction between Intermediate 29 and 2-nitrobenzenesulfonyl chloride can be carried out under suitable conditions (such as, e.g. triethylamine in DCM) to give Intermediate 32. Intermediate 30A can be reacted with p-toluenesulfonyl chloride under suitable conditions to give Intermediate 33. Intermediate 32 can be reacted with Intermediate 33 under suitable conditions (such as, e.g. K₂CO₃, BnBr, and thiophenol) to give Intermediate 31.

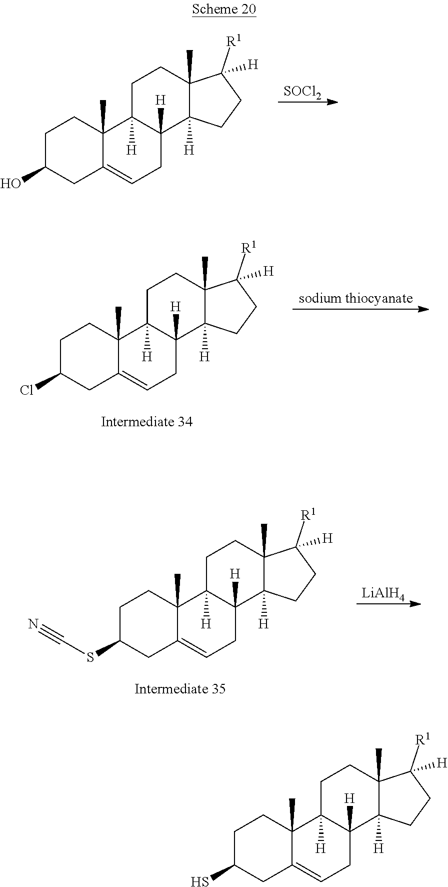

Intermediates for the synthesis of compounds of Formula A1 can be prepared via the synthetic route outlined in Scheme 20. An appropriate reaction between cholesterol or a cholesterol derivative (such as, sitosterol) and thionyl chloride under suitable conditions affords Intermediate 34. Intermediate 34 can be reacted with sodium thiocyanate under suitable conditions to give Intermediate 35. Intermediate 35 can be reacted with lithium aluminum hydride under suitable conditions to give thiocholesterol or a thiocholesterol derivative (such as, thiositosterol).

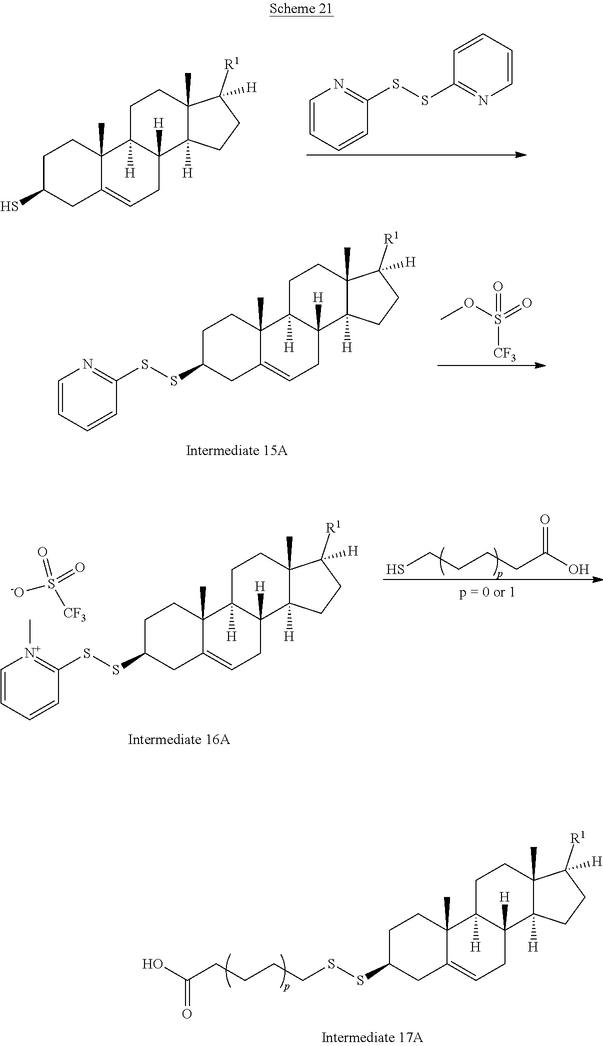

Intermediates for the synthesis of compounds of Formula A1 can be prepared via the synthetic route outlined in Scheme 21. An appropriate reaction between thiocholesterol or a thiocholesterol derivative (such as, thiositosterol) and 2,2′-dipyridyldisulfide under suitable conditions give Intermediate 15A. Intermediate 15A can be reacted with methyl trifluoromethanesulfonate (methyl triflate) under suitable conditions to give Intermediate 16A. Intermediate 16A can be reacted with an appropriate mercaptocarboxylic acid to afford Intermediate 17A.

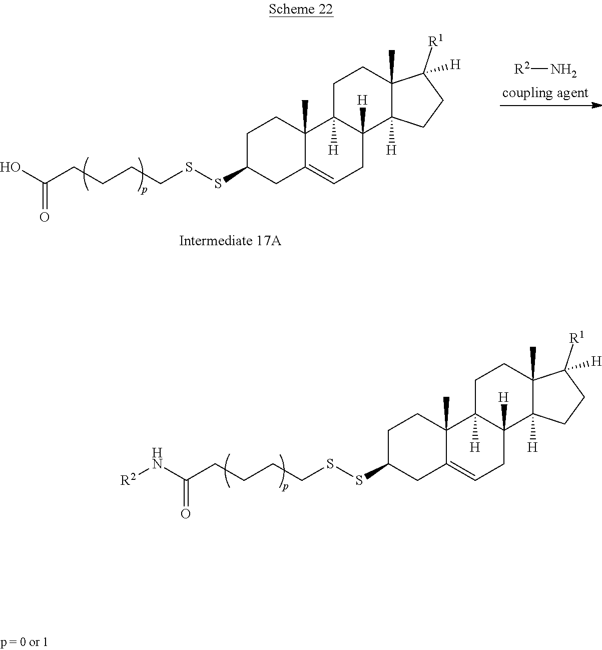

Compounds of Formula A1 can be prepared via the synthetic route outlined in Scheme 22. An appropriate reaction between Intermediate 17A and an amine can be carried out under suitable conditions (such as using a coupling agent) to generate a precursor to a compound of Formula A1.

### Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the present disclosure. Where a value is explicitly recited, it is to be understood that values which are about the same quantity or amount as the recited value are also within the scope of the present disclosure. Where a combination is disclosed, each subcombination of the elements of that combination is also specifically disclosed and is within the scope of the present disclosure. Conversely, where different elements or groups of elements are individually disclosed, combinations thereof are also disclosed. Where any element of an present disclosure is disclosed as having a plurality of alternatives, examples of that present disclosure in which each alternative is excluded singly or in any combination with the other alternatives are also hereby disclosed; more than one element of an present disclosure can have such exclusions, and all combinations of elements having such exclusions are hereby disclosed.

The term “about” as used in connection with a numerical value throughout the specification and the claims denotes an interval of accuracy, familiar and acceptable to a person skilled in the art. Such interval of accuracy is, for example, +10%.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a cell (e.g., a mammalian cell) with a nanoparticle composition means that the cell and a nanoparticle are made to share a physical connection. Methods of contacting cells with external entities both in vivo and ex vivo are well known in the biological arts. For example, contacting a nanoparticle composition and a cell disposed within a mammal can be performed by varied routes of administration (e.g., intravenous, intramuscular, intradermal, and subcutaneous) and can involve varied amounts of nanoparticle compositions. Moreover, more than one cell can be contacted by a nanoparticle composition.

A further example of contacting is between a nanoparticle and a lipid amine. Contacting a nanoparticle (e.g., filled with payload or empty) and a lipid amine can mean that the surface of the nanoparticle is put in physical connection with the lipid amine so that, the lipid amine can form an interaction with the nanoparticle. In some embodiments, contacting a nanoparticle and a lipid amine results in intercalation of the lipid amine into the nanoparticle, for example, starting at the surface of the nanoparticle. In some embodiments, the terms “layering,” “coating,” and “post addition” and “addition” can be used to mean “contacting” in reference to contacting a nanoparticle with a lipid amine.

As used herein, the term “delivering” means providing an entity to a destination. For example, delivering a polynucleotide to a subject can involve administering a nanoparticle composition including the polynucleotide to the subject (e.g., by an intravenous, intramuscular, intradermal, or subcutaneous route). Administration of a nanoparticle composition to a mammal or mammalian cell can involve contacting one or more cells with the nanoparticle composition.

As used herein, “delivery agent” refers to any substance that facilitates, at least in part, the in vivo, in vitro, or ex vivo delivery of a polynucleotide to targeted cells.

As used herein, the term “diastereomer,” means stereoisomers that are not mirror images of one another and are non-superimposable on one another.

As used herein, the term “effective amount” of an agent is that amount sufficient to effect beneficial or desired results, for example, clinical results, and, as such, an “effective amount” depends upon the context in which it is being applied. For example, in the context of administering an agent that treats a protein deficiency, an effective amount of an agent is, for example, an amount of mRNA expressing sufficient protein to ameliorate, reduce, eliminate, or prevent the signs and symptoms associated with the protein deficiency, as compared to the severity of the symptom observed without administration of the agent. The term “effective amount” can be used interchangeably with “effective dose,” “therapeutically effective amount,” or “therapeutically effective dose.”

As used herein, the term “enantiomer” means each individual optically active form of a compound of the present disclosure, having an optical purity or enantiomeric excess (as determined by methods standard in the art) of at least 80% (i.e., at least 90% of one enantiomer and at most 10% of the other enantiomer), at least 90%, or at least 98%.

As used herein, the term “encapsulate” means to enclose, surround, incorporate, or encase.

As used herein, “encapsulation efficiency” refers to the amount of a polynucleotide that becomes part of a nanoparticle composition, relative to the initial total amount of polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency can be given as 97%. As used herein, “encapsulation” can refer to complete, substantial, or partial enclosure, confinement, surrounding, or encasement.

As used herein, “epithelial cells” include cells derived from epithelium. Example epithelial cells are respiratory epithelial cells, nasal epithelial cells, alveolar epithelial cells, lung epithelial cells, or bronchial epithelial cells. In some embodiments, the epithelial cells are human bronchial epithelial (HBE) cells. In some embodiments, epithelial cells are in vitro cells. In some embodiments, epithelial cells are in vivo cells.

As used herein, “expression” of a nucleic acid sequence refers to one or more of the following events: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the bilayer and/or to help facilitate endosomal escape, e.g., of nucleic acid delivered to cells. Helper lipids are also believed to be a key structural component to the surface of the LNP.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

The term “ionizable amino lipid” includes those lipids described herein throughout that, for example, exhibit one, two, three, or more fatty acid or fatty alkyl chains and at least one pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z, 165Z)-N,N-dimethyl-3-nonydocosa-13-16-dien-1-amine (L608).

As used herein, the term “isomer” means any tautomer, stereoisomer, enantiomer, or diastereomer of any compound of the present disclosure. It is recognized that the compounds of the present disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the present disclosure, the chemical structures depicted herein, and therefore the compounds of the present disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, a “lipid nanoparticle core” is a lipid nanoparticle to which post addition layers of additional components can be added, such as a lipid amine and/or a PEG-lipid or other lipid. In some embodiments, the lipid nanoparticle core comprises: (i) an ionizable lipid, (ii) a phospholipid, (iii) a structural lipid, and (iv) optionally a PEG-lipid. In further embodiments, the lipid nanoparticle core comprises: (i) an ionizable lipid, (ii) a phospholipid, (iii) a structural lipid, and (iv) a PEG-lipid. In some embodiments, the lipid nanoparticle core can contain payload.

As used herein, a “linker” or “linker structure” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

As used herein, “lung cells” include cells derived from the lungs. Lung cells can be, for example, lung epithelial cells, airway basal cells, bronchiolar exocrine cells, pulmonary neuroendocrine cells, alveolar cells, or airway epithelial cells. In some embodiments, lung cells are in vitro cells. In some embodiments, lung cells are in vivo cells.

The term “nucleic acid,” in its broadest sense, includes any compound and/or substance that comprises a polymer of nucleotides. These polymers are often referred to as polynucleotides. Exemplary nucleic acids or polynucleotides of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

As used herein, “patient” refers to a subject who can seek or be in need of treatment, requires treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration.

The “pharmaceutically acceptable salts” of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “solvate,” as used herein, means a compound of the present disclosure wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. When water is the solvent, the solvate is referred to as a “hydrate.”

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding mRNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ‘P’PC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include encoded polynucleotide products, naturally occurring polypeptides, synthetic polypeptides, homologs, orthologs, paralogs, fragments and other equivalents, variants, and analogs of the foregoing. A polypeptide can be a monomer or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

As used herein, the term “preventing” refers to partially or completely delaying onset of an infection, disease, disorder and/or condition; partially or completely delaying onset of one or more signs and symptoms, features, or clinical manifestations of a particular infection, disease, disorder, and/or condition; partially or completely delaying onset of one or more signs and symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “prophylactic” refers to a therapeutic or course of action used to prevent the spread of disease.

The term “salt” includes any anionic and cationic complex. Pharmaceutically acceptable salts represent a subset of non-toxic salts as described hereinabove.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of treatment.

As used herein, the term “substantially” refers to the qualitative condition of exhibiting total or near-total extent or degree of a characteristic or property of interest. One of ordinary skill in the biological arts will understand that biological and chemical characteristics rarely, if ever, go to completion and/or proceed to completeness or achieve or avoid an absolute result. The term “substantially” is therefore used herein to capture the potential lack of completeness inherent in many biological and chemical characteristics.

An individual who is “suffering from” a disease, disorder, and/or condition has been diagnosed with or displays one or more signs and symptoms of the disease, disorder, and/or condition.

An individual who is “susceptible to” a disease, disorder, and/or condition has not been diagnosed with and/or cannot exhibit signs and symptoms of the disease, disorder, and/or condition but harbors a propensity to develop a disease or its signs and symptoms. In some embodiments, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or other molecules of the present disclosure can be chemical or enzymatic.

The term “therapeutic or prophylactic agent” refers to an agent that, when administered to a subject, has a therapeutic, diagnostic, and/or prophylactic effect and/or elicits a desired biological and/or pharmacological effect. For example, an mRNA encoding a polypeptide can be a therapeutic or prophylactic agent.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve signs and symptoms of, diagnose, prevent, and/or delay the onset of the infection, disease, disorder, and/or condition.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more signs and symptoms or features of a disease. For example, “treating” a disease can refer to diminishing signs and symptoms associated with the disease, prolonging the lifespan (increase the survival rate) of patients, reducing the severity of the disease, preventing or delaying the onset of the disease, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. As used herein, the term “Cₙ₋ₘ hydroxyalkyl” refers to an alkyl group substituted with a hydroxy (—OH) group.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphthyl.

As used herein, “halo” refers to F, Cl, Br, or I. In some embodiments, a halo is F, Cl, or Br. In some embodiments, the halo is F.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, a “bridged ring” or a “bridged ring group” is cyclic system with at least two joined rings that share three or more atoms. A bridged ring can be a carbocycle ring or a heterocycloalkyl ring. Example bridged rings include

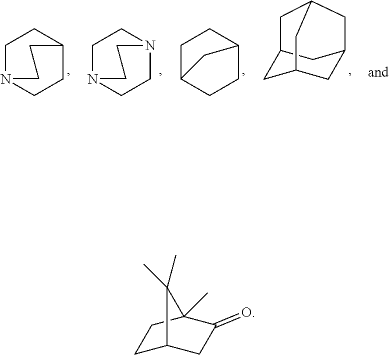

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the present disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Abbreviations

ACN: Acetonitrile
Aq.: Aqueous
Boc₂O: Di-tert-butyl pyrocarbonate
DBU: 1,8-Diazabicyclo[5.4.0]undec-7-ene
BnBr: Benzyl bromide
DCC: N,N′-Dicyclohexylcarbodiimide
DCM: Dichloromethane
DMAP: 4-Dimethylaminopyridine
DMF: Dimethylformamide
EtOAc: Ethyl acetate
h: hour/hours
LCMS: Liquid chromatography-mass spectrometry
MTBE: Methyl tert-butyl ether
PMA: Phosphomolybdic Acid
Soln.: Solution
rt: room temperature
THF: Tetrahydrofuran
TLC: Thin layer chromatography

### Example 1

### Synthesis of Compounds According to Formula A1

### Intermediate A: tert-butyl (5-((3-((tert-butoxycarbonyl)amino)butyl)amino)pentan-2-yl)carbamate

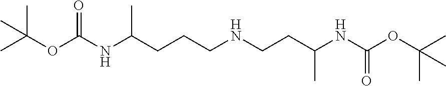 [SMILES: CC(CCCNCCC(C)NC(=O)OC(C)(C)C)NC(=O)OC(C)(C)C]

### Step 1: tert-butyl (5-hydroxypentan-2-yl)carbamate

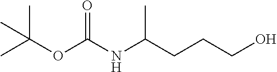

To a solution of di-tert-butyl dicarbonate (2.45 mL, 10.66 mmol) in dry DCM (30 mL) stirring under nitrogen at 0° C., was added 4-aminopentan-1-ol (1.00 g, 9.69 mmol) dropwise over 10 minutes. The solution was allowed to gradually warm to room temperature and continue stirring over 3 days. Following, then solvent was removed under vacuum, and the resulting residue was taken up in DCM and purified on silica in hexanes with a 0-100% EtOAc gradient. Product-containing fractions were pooled and concentrated in vacuo to give tert-butyl (5-hydroxypentan-2-yl)carbamate as an oil (0.86 g, 4.23 mmol, 43.6%). UPLC/ELSD: RT=0.28 min. MS (ES): m/z (MH⁺) 204.3 for C₁₀H₂₁NO₃. ¹H NMR (300 MHz, CDCl₃) δ 4.51 (br. s, 1H), 3.62 (t, 3H), 2.59 (br. s, 1H), 1.53 (m, 4H), 1.42 (s, 9H), 1.12 (d, 3H, J=6 Hz).

### Step 2: 4-((tert-butoxycarbonyl)amino)pentyl 4-methylbenzenesulfonate

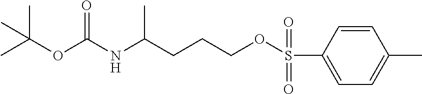 [SMILES: Cc1ccc(S(=O)(=O)OCCCC(C)NC(=O)OC(C)(C)C)cc1]

To a solution of tert-butyl (5-hydroxypentan-2-yl)carbamate (0.86 g, 4.23 mmol) in dry DCM (30 mL) set stirring under nitrogen, was added triethylamine (2.95 mL, 21.15 mmol), dimethylaminopyridine (0.10 g, 0.85 mmol), and p-toluenesulfonyl chloride (1.61 g, 8.46 mmol). The solution was allowed to stir at room temperature overnight, over which it turned a dark red color. The mixture was then further diluted with DCM, washed with water (1×30 mL), saturated aqueous sodium bicarbonate (1×30 mL) and brine (1×30 mL), dried over sodium sulfate, filtered, and concentrated to a dark brown oil. The oil was taken up in DCM and purified on silica in hexanes with a 0-50% EtOAc gradient. Product-containing fractions were pooled and concentrated to give 4-((tert-butoxycarbonyl)amino)pentyl 4-methylbenzenesulfonate as a light brown oil (0.97 g, 2.72 mmol, 64.3%). UPLC/ELSD: RT: 1.02 min. MS (ES): m/z (MH⁺) 358.4 for C₁₇H₂₇NO₅S. ¹H NMR (300 MHz, CDCl₃) δ 7.77 (d, 2H, J=6 Hz), 7.36 (d, 2H, J=6 Hz), 4.32 (br. s, 1H), 4.03 (t, 2H), 3.55 (br. s, 1H), 2.45 (s, 3H), 1.69 (br. m, 2H), 1.41 (s, 11H), 1.06 (d, 3H, J=6 Hz).

### Step 3: tert-butyl (5-((3-((tert-butoxycarbonyl)amino)butyl)amino)pentan-2-yl)carbamate

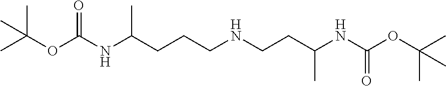 [SMILES: CC(CCCNCCC(C)NC(=O)OC(C)(C)C)NC(=O)OC(C)(C)C]

To a solution of tert-butyl N-[4-(2-nitrobenzenesulfonamido)butan-2-yl]carbamate (0.97 g, 2.61 mmol) in dry DMF (20 mL) stirring at room temperature under nitrogen, was added 4-((tert-butoxycarbonyl)amino)pentyl 4-methylbenzenesulfonate (1.03 g, 2.87 mmol) and potassium carbonate (1.08 g, 7.82 mmol). The solution was warmed to 100° C. and stirred for 48 hours. Then, the reaction was quenched with 30 mL water and diluted with 50 mL EtOAc. The organic layer was separated, and the aqueous layer was extracted with EtOAc (3×30 mL). The organic layers were all combined, washed with water (1×50 mL) and brine (1×50 mL), dried over sodium sulfate, filtered, and concentrated to a yellow oil. The oil was taken up in DCM and purified on silica in hexanes with a 0-100% EtOAc gradient. Product-containing fractions were pooled and concentrated to an oil. The oil was taken up in 20 mL DMF, to which was added thiophenol (1.03 mL, 10.03 mmol) and potassium carbonate (1.08 g, 7.82 mmol). The solution stirred at room temperature overnight. The next morning, salts were removed from the mixture by centrifugation and the supernatant was concentrated to a residue. The residue was taken up in 40 mL DCM, washed with water (2×10 mL) and brine (2×10 mL), dried over potassium carbonate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give tert-butyl (5-((3-((tert-butoxycarbonyl)amino)butyl)amino)pentan-2-yl)carbamate as a colorless oil (0.34 g, 0.91 mmol, 34.93%). UPLC/ELSD: RT: 0.28 min. MS (ES): m/z (MH⁺) 374.4 for C₁₉H₃₉N₃O₄. ¹H NMR (300 MHz, CDCl₃) δ 4.91 (br. m, 1H), 4.67 (br. m, 1H), 3.70 (br. m, 2H), 3.40 (s, 2H), 2.72 (t, 1H), 2.58 (br. m, 3H), 2.41 (br. s, 4H), 1.50 (br. m, 5H), 1.41 (s, 19H), 1.10 (d, 6H, J=6 Hz).

### A. Compound SA50: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride

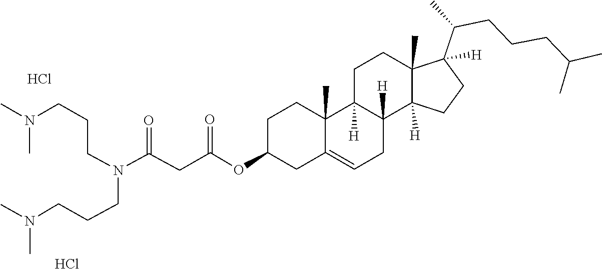

### Step 1: tert-Butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) malonate

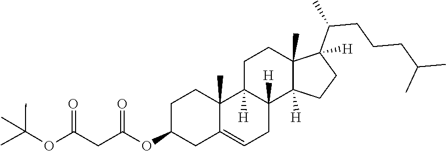

To a solution of cholesterol (4.00 g, 10.14 mmol) and 3-(tert-butoxy)-3-oxopropanoic acid (2.39 mL, 15.21 mmol) in dichloromethane (20 mL) stirring under nitrogen was added 1-ethyl-3-(3-dimethylaminopropyl)carbodiimide hydrochloride (2.95 g, 15.21 mmol). Then the reaction mixture was cooled to 0° C., and diisoproylethylamine (5.36 mL, 30.41 mmol) was added dropwise over 20 minutes. The resulting mixture was allowed to gradually warm to room temperature and proceed overnight. The mixture was then diluted with dichloromethane to 150 mL, washed with water (1×70 mL), saturated aqueous sodium bicarbonate (2×70 mL), and brine (1×70 mL), dried over sodium sulfate, filtered, and concentrated in vacuo to give a yellow oil. The oil was taken up in dichloromethane and purified on silica with a 0-25% ethyl acetate gradient in hexanes to give tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) malonate (4.99 g, 9.44 mmol, 93.1%) as an oil. UPLC/ELSD: RT: 3.36 min. MS (ES): m/z (MH⁺) 529.8 for C₃₄H₅₆O₄. ¹H NMR (300 MHz, CDCl₃) δ: ppm 5.41 (m, 1H), 4.67 (m, 1H), 3.27 (s, 2H), 2.38 (d, 2H), 1.91 (br. m, 10H), 1.49 (s, 12H), 1.35 (br. m, 6H), 1.04 (br. m, 17H), 0.91 (d, 3H, J=3 Hz), 0.87 (d, 3H, J=3 Hz), 0.70 (s, 3H).

### Step 2: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid

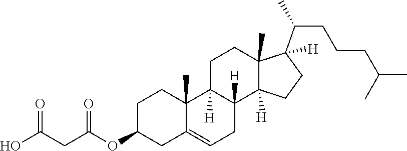

To a solution of tert-butyl ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) malonate (4.99 g, 9.44 mmol) in dichloromethane (50 mL) stirring under nitrogen at 0° C., was added trifluoroacetic acid (10.85 mL, 141.63 mmol) dropwise over 20 minutes. The clear, light yellow reaction mixture was allowed to gradually warm to room temperature and proceed overnight. The following morning, the reaction was quenched with 20 mL of a 5% aqueous sodium bicarbonate solution at 0° C. The organics were separated, washed with an additional 10 mL of 5% aqueous sodium bicarbonate, dried over sodium sulfate, filtered, and concentrated to give a white solid. The solid was taken up in dichloromethane and purified on silica with a 0-60% ethyl acetate gradient in hexanes to give 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (3.12 g, 6.61 mmol, 70.0%) as a white solid. UPLC/ELSD: RT: 2.97 min. MS (ES): m/z (MH⁺) 473.7 for C₃₀H₄₈O₄. ¹H NMR (300 MHz, CDCl₃) δ: ppm 10.99 (br. s, 1H), 5.42 (m, 1H), 4.73 (m, 1H), 3.45 (s, 2H), 2.37 (d, 2H, J=9 Hz), 1.89 (br. m, 5H), 1.35 (br. m, 18H), 1.05 (s, 5H), 0.94 (d, 4H, J=2 Hz), 0.89 (d, 6H, J=2 Hz), 0.70 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate

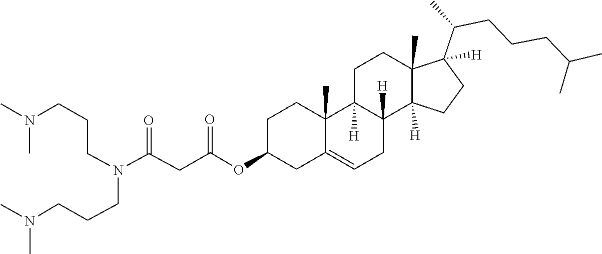

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (3.12 g, 6.61 mmol) in dichloromethane (60 mL) stirring under nitrogen was added tetramethyldipropylenetriamine (2.30 mL, 9.81 mmol), dimethylaminopyridine (0.08 g, 0.65 mmol), and 1-ethyl-3-(3-dimethylaminopropyl)carbodiimide hydrochloride (1.90 g, 9.81 mmol). The reaction mixture was cooled to 0° C. and diisopropylethylamine (3.46 mL, 19.62 mmol) was added dropwise over 20 minutes. The mixture was allowed to gradually warm to room temperature and proceed overnight. The solution was then diluted with dichloromethane, washed with saturated aqueous sodium bicarbonate (1×50 mL) and brine (1×50 mL), dried over sodium sulfate, filtered, and concentrated to an oil. The oil was taken up in dichloromethane and purified on silica with a 0-60% (9:1 methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate (1.82 g, 2.84 mmol, 43.4%) as a yellow oil. UPLC/ELSD: RT: 1.85 min. MS (ES): m/z (MH⁺) 643.0 for C₄₀H₇₁N₃O₃. ¹H NMR (300 MHz, CDCl₃) δ: ppm 5.39 (m, 1H), 4.67 (m, 1H), 3.54 (s, 2H), 3.35 (br. m, 4H), 2.37 (br. m, 6H), 2.22 (d, 12H, J=3 Hz), 1.50 (br. m, 28H), 1.02 (br. s, 5H), 0.92 (d, 4H, J=6 Hz), 0.88 (d, 6H, J=9 Hz), 0.68 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride

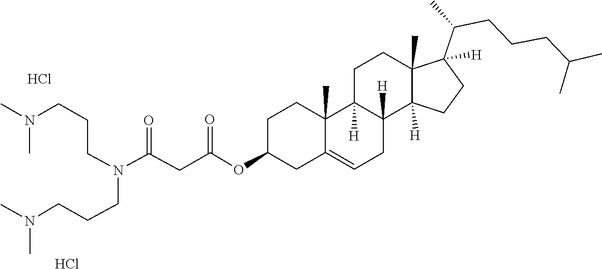

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate (0.22 g, 0.32 mmol) in diethyl ether (4.3 mL) and isopropanol (0.22 mL) was added hydrochloric acid (5.5 M in isopropanol, 0.37 mL, 1.85 mmol) dropwise. The mixture was cooled to 0° C. and stirred vigorously for 30 minutes, after which the white precipitate was filtered out via vacuum filtration and washed repeatedly with cold ether. The residue was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(bis(3-(dimethylamino)propyl)amino)-3-oxopropanoate dihydrochloride as a white waxy solid (0.11 g, 0.15 mmol, 46.6%). UPLC/ELSD: RT: 1.81 min. MS (ES): m/z (MH⁺) 627.99 for C₄₀H₇₃Cl₂N₃O₃. ¹H NMR (300 MHz, CD₃OD) δ: ppm 5.41 (br. s, 1H), 4.64 (br. m, 1H), 3.57 (br. m, 7H), 3.33 (br. s, 2H), 3.20 (br. m, 5H), 2.93 (d, 15H, J=6 Hz), 2.40 (d, 2H, J=9 Hz), 2.05 (br. m, 12H), 1.55 (br. m, 14H), 1.20 (br. m, 13H), 1.07 (s, 7H), 0.98 (d, 5H, J=6 Hz), 0.91 (d, 7H, J=6 Hz), 0.74 (s, 3H).

### B. Compound SA51: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride

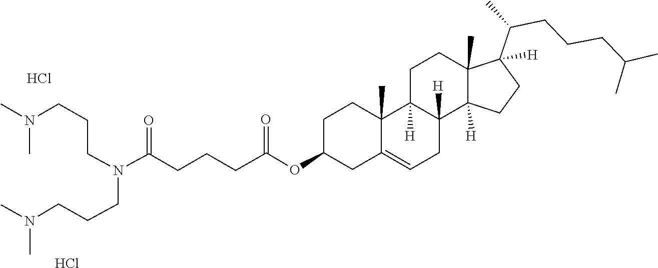

### Step 1: 5-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid

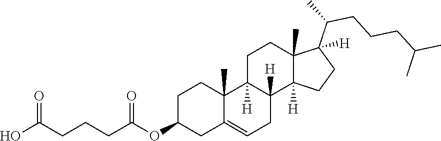

To a solution of cholesterol (5.00 g, 12.67 mmol) in acetone (50 mL) stirring under nitrogen was added glutaric anhydride (2.63 g, 22.81 mmol) and triethylamine (3.21 mL, 22.81 mmol). The reaction mixture was refluxed at 56° C., turning from a white slurry to a colorless clear solution, and allowed to proceed at reflux for 3 days. Following, the solution was cooled to room temperature, concentrated under vacuum, and taken up in 150 mL dichloromethane. This was then washed with 0.5 M HCl (1×100 mL) and saturated aqueous ammonium chloride (1×100 mL), dried over sodium sulfate, filtered, and concentrated to give a white solid. The solid was taken up in dichloromethane and purified on silica with a 0-50% ethyl acetate gradient in hexanes to give 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (6.011 g, 12.00 mmol, 94.7%) as a white solid. UPLC/ELSD: RT: 2.96 min. MS (ES): m/z (MH⁺) 501.7 for C₃₂H₅₂O₄. ¹H NMR (300 MHz, CDCl₃) δ: ppm 5.40 (m, 1H), 4.66 (m, 1H), 2.45 (br. m, 5H), 2.01 (br. m, 3H), 1.85 (br. m, 3H), 1.34 (br. m, 22H), 0.94 (d, 3H, J=6 Hz), 0.88 (d, 6H, J=9 Hz), 0.70 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate

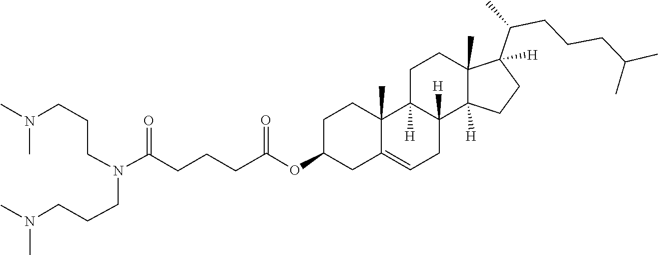

To a solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (6.01 g, 11.88 mmol) in dichloromethane (100 mL) stirring under nitrogen was added tetramethyldipropylenetriamine (4.19 mL, 17.82 mmol), dimethylaminopyridine (0.15 g, 1.19 mmol), and 1-ethyl-3-(3-dimethylaminopropyl)carbodiimide hydrochloride (3.45 g, 17.83 mmol). The solution was cooled to 0° C., and then diisopropylethylamine (6.29 mL, 35.65 mmol) was added dropwise. The reaction mixture was allowed to gradually warm to room temperature and proceed overnight. The solution was diluted further with dichloromethane, washed with saturated aqueous sodium bicarbonate (1×100 mL) and brine (1×100 mL), dried over sodium sulfate, filtered, and concentrated to give an oil. The material was taken up in dichloromethane and purified on silica in a 0-60% (9:1 methanol: aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate (2.30 g, 11.88 mmol, 28.9%) as an oil. UPLC/ELSD: RT: 2.02 min. MS (ES): m/z (MH⁺) 671.1 for C₄₂H₇₅N₃O₃. ¹H NMR (300 MHz, CDCl₃) δ: ppm 5.36 (m, 1H), 4.60 (m, 1H), 3.33 (br. m, 5H), 2.39 (br. m, 12H), 2.23 (d, 11H, J=6 Hz), 1.99 (br. m, 4H), 1.84 (br. m, 3H), 1.71 (br. m, 5H), 1.33 (br. m, 11H), 1.14 (br. m, 7H), 1.02 (s, 6H), 0.92 (d, 3H, J=6 Hz), 0.87 (d, 5H, J=9 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride

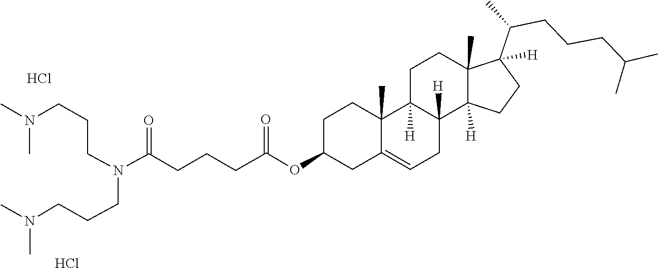

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate (0.23 g, 0.33 mmol) in diethyl ether (4.6 mL) and isopropanol (0.23 mL) was added hydrochloric acid (5.5M in isopropanol, 0.37 mL, 1.85 mmol) dropwise. The mixture was cooled to 0° C. and stirred vigorously for 30 minutes, after which the white precipitate was filtered out via vacuum filtration and washed repeatedly with cold ether. The residue was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-(bis(3-(dimethylamino)propyl)amino)-5-oxopentanoate dihydrochloride as a white waxy solid (0.12 g, 0.16 mmol, 49.5%). UPLC/ELSD: RT: 1.86 min. MS (ES): m/z (MH⁺) 671.81 for C₄₂H₇₇Cl₂N₃O₃. ¹H NMR (300 MHz, CD₃OD) δ: ppm 5.41 (br. s, 1H), 4.55 (br. m, 1H), 3.54 (t, 5H, J=6 Hz), 3.24 (br. m, 6H), 2.94 (d, 14H, J=6 Hz), 2.55 (t, 2H, J=6 Hz), 2.43 (t, 2H, J=6 Hz), 2.35 (d, 2H, J=9 Hz), 2.05 (br. m, 6H), 1.90 (br. m, 6H), 1.55 (br. m, 12H), 1.19 (br. m, 10H), 1.07 (s, 7H), 0.98 (d, 4H, J=6 Hz), 0.90 (d, 7H, J=6 Hz), 0.74 (s, 3H).

### C. Compound SA56: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate

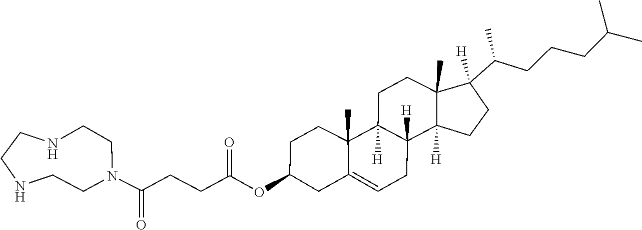

### Step 1: Di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate

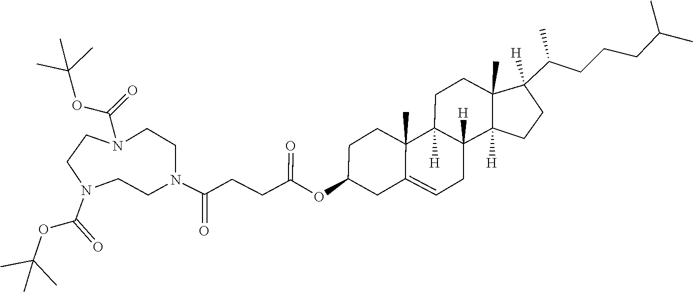

To a stirred solution of cholesteryl hemisuccinate (100 mg, 0.205 mmol), 1,4-di-tert-butyl 1,4,7-triazonane-1,4-dicarboxylate (Enamine, Monmouth Junction, NJ) (0.068 g, 0.20 mmol), and DMAP (cat.) in DCM (1.4 mL) was added 1-ethyl-3-(3-dimethylaminopropyl)carbodiimide hydrochloride (0.060 g, 0.31 mmol). The reaction mixture stirred at rt and was monitored by TLC. At 21.5 h water (1.5 mL) was added. After stirring for 16 h additional water (10 mL) was added. The mixture was then extracted with DCM (2×15 mL). The combined organics were passed through a hydrophobic frit, dried over Na₂SO₄, and then concentrated. The crude material was purified via silica gel chromatography (0-4% MeOH in DCM) to afford di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate (130 mg, 0.163 mmol, 79.3%) as a clear oil. UPLC/ELSD: RT=3.41 min. MS (ES): m/z=1619.2 [2M+Na]⁺ for C₄₇H₇₉N₃O₇; ¹H NMR (300 MHz, CDCl₃): δ 5.33-5.39 (m, 1H), 4.52-4.68 (m, 1H), 3.18-3.79 (br. m, 12H), 2.49-2.71 (m, 4H), 2.24-2.39 (m, 2H), 1.74-2.06 (br. m, 5H), 0.93-1.71 (br. m, 39H), 1.01 (s, 3H), 0.91 (d, 3H, J=6.5 Hz), 0.87 (d, 3H, J=6.6 Hz), 0.86 (d, 3H, J=6.5 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride

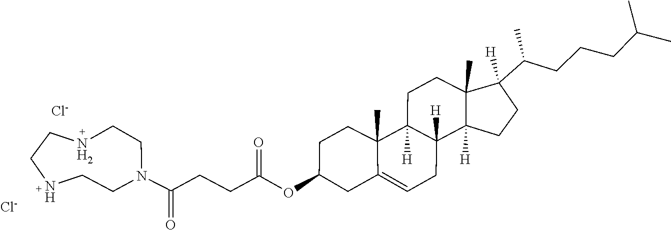

To a solution of di-tert-butyl 7-(4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoyl)-1,4,7-triazonane-1,4-dicarboxylate (123 mg, 0.154 mmol) in iPrOH (2.0 mL) was added 5-6 N HCl in iPrOH (0.18 mL). The reaction mixture stirred at 40° C. and was monitored by LCMS. At 17 h, additional iPrOH (2.0 mL) and 5-6 N HCl in iPrOH (0.06 mL) were added. At 41 h, the reaction mixture was cooled to rt, and ACN (4 mL) was added. Solids were collected by vacuum filtration and rinsed with ACN to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride (0.082 g, 0.11 mmol, 73.8%) as a white solid. UPLC/ELSD: RT=2.30 min. MS (ES): m/z=598.1 [M+H]⁺ for C₃₇H₆₃N₃O₃; ¹H NMR (300 MHz, CDCl₃): δ 10.36 (br. s, 2H), 10.11 (br. s, 2H), 5.34-5.43 (m, 1H), 4.50-4.65 (m, 1H), 3.97-4.29 (m, 4H), 3.64-3.95 (m, 6H), 3.41-3.61 (m, 2H), 2.66-2.84 (m, 2H), 2.45-2.65 (m, 2H), 2.21-2.40 (m, 2H), 1.75-2.08 (br. m, 5H), 0.94-1.70 (br. m, 21H), 1.01 (s, 3H), 0.91 (d, 3H, J=6.4 Hz), 0.87 (d, 3H, J=6.5 Hz), 0.86 (d, 3H, J=6.6 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate

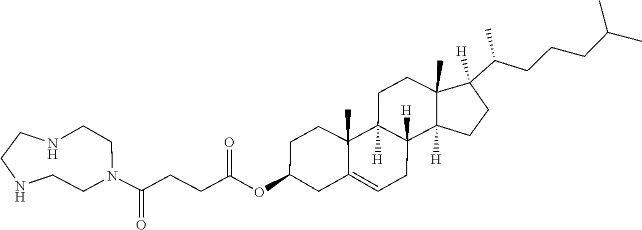

(3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate dihydrochloride (0.054 g, 0.075 mmol) was suspended in 5% aq. NaHCO₃ solution (10 mL) and then extracted with DCM (3×10 mL). K₂CO₃ (ca. 100 mg) was added to the aqueous layer. The aqueous layer was extracted with DCM (2×10 mL). The combined organics were passed through a hydrophobic frit, dried over Na₂SO₄, and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-oxo-4-(1,4,7-triazonan-1-yl)butanoate (0.023 g, 0.037 mmol, 50.0%) as a white solid. ¹H NMR (300 MHz, CDCl₃): δ 5.33-5.40 (m, 1H), 4.54-4.70 (m, 1H), 3.41-3.57 (m, 4H), 2.99-3.15 (m, 4H), 2.72-2.84 (m, 4H), 2.58-2.72 (m, 4H), 2.24-2.39 (m, 2H), 1.74-2.19 (br. m, 7H), 0.94-1.70 (br. m, 21H), 1.01 (s, 3H), 0.91 (d, 3H, J=6.5 Hz), 0.87 (d, 3H, J=6.6 Hz), 0.86 (d, 3H, J=6.6 Hz), 0.67 (s, 3H). UPLC/ELSD: RT=2.39 min. MS (ES): m/z=598.6 [M+H]⁺ for C₃₇H₆₃N₃O₃.

### D. Compound SA57: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate

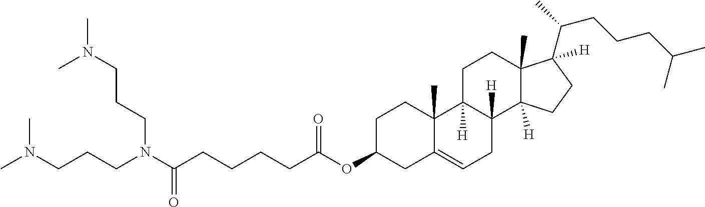

### Step 1: 6-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid

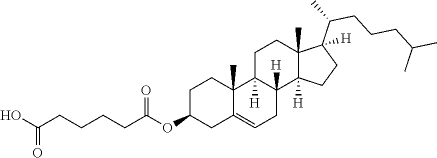

To a solution of cholesterol (5.00 g, 12.93 mmol) in dichloromethane (50 mL) stirring under nitrogen was added adipic anhydride (1.66 g, 12.93 mmol). Then, pyridine (3.97 mL, 28.45 mmol) was added dropwise over 10 minutes. The reaction mixture was heated to a reflux at 40° C. and proceeded overnight. Then, the mixture was allowed to cool to room temperature and concentrated to a yellow oil. The oil was taken up in dichloromethane and purified on silica without additional workup in a 0-30% ethyl acetate gradient in hexanes to give 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (1.97 g, 3.83 mmol, 29.6%) as a white solid. UPLC/ELSD: RT: 3.09 min. MS (ES): m/z (MH⁺) 515.7 for C₃₃H₅₄O₄. ¹H NMR (300 MHz, CDCl₃) δ: ppm 12.15 (br. s, 1H), 5.39 (m, 1H), 4.63 (br. m, 1H), 2.40 (br. m, 6H), 2.00 (br. m, 2H), 1.85 (br. m, 3H), 1.70 (br. m, 4H), 1.34 (br. m, 19H), 1.03 (s, 6H), 0.93 (d, 4H, J=6 Hz), 0.88 (d, 6H, J=6 Hz), 0.69 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate

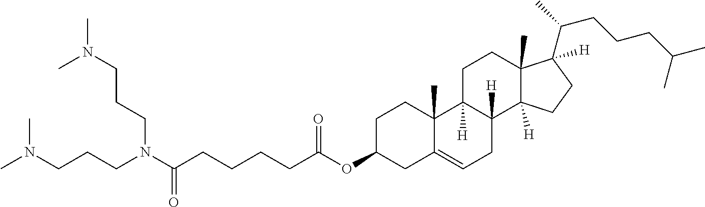

To a solution of 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (1.00 g, 1.92 mmol) in dichloromethane (25 mL) stirring under nitrogen was added tetramethyldipropylenetriamine (0.68 mL, 2.89 mmol), dimethylaminopyridine (0.02 g, 0.19 mmol), and 1-ethyl-3-(3-dimethylaminopropyl)carbodiimide hydrochloride (0.56 g, 2.89 mmol). The resulting solution was cooled to 0° C. and diisopropylethylamine (1.02 mL, 5.77 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-60% (8:2:0.1 dichloromethane/methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate as a yellow oil by ¹H NMR, so the material was purified again on silica using a 0-25% (8:2:0.1 dichloromethane/methanol/conc. aqueous ammonium hydroxide) gradient in dichloromethane to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-(bis(3-(dimethylamino)propyl)amino)-6-oxohexanoate as a light yellow oil (0.38 g, 0.54 mmol, 28.2%). UPLC/ELSD: RT: 2.11 min. MS (ES): m/z (MH⁺) 685.1 for C₄₃H₇₇N₃O₃. ¹H NMR (300 MHz, CDCl₃) δ: ppm 5.40 (m, 3H), 5.20 (m, 1H), 4.40 (br. m, 1H), 3.22 (m, 4.48), 2.58 (s, 3H), 2.38 (t, 2H, J=9 Hz), 2.26 (s, 6H), 2.20 (d, 3H, J=9 Hz), 2.14 (br. s, 9H), 1.49 (br. m, 24H), 0.95 (br. m, 7H), 0.85 (s, 5H), 0.75 (d, 4H, J=6 Hz), 0.70 (d, 5H, J=9 Hz), 0.51 (s, 3H).

### E. Compound SA58: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

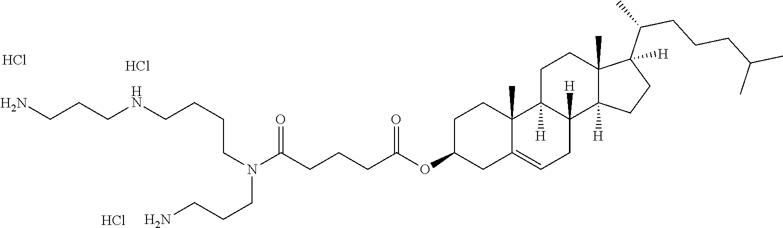

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate

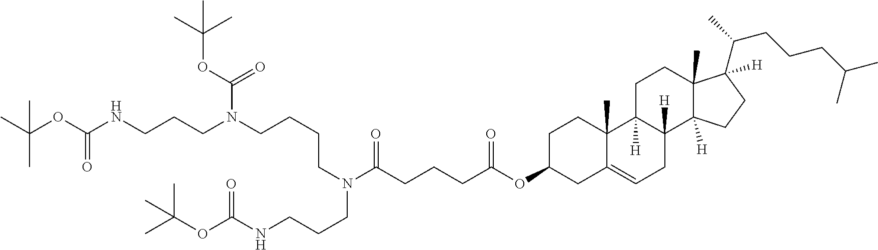

To a solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.76 g, 1.49 mmol) in dichloromethane (20 mL) stirring under nitrogen was added tert-butyl (3-((tert-butoxycarbonyl)amino)propyl)(4-((3-((tert-butoxycarbonyl)amino)propyl)amino)butyl)carbamate (0.75 g, 1.49 mmol), dimethylaminopyridine (0.02 g, 0.15 mmol), and 1-ethyl-3-(3-dimethylaminopropyl)carbodiimide hydrochloride (0.43 g, 2.24 mmol). The resulting solution was cooled to 0° C. and diisopropylethylamine (0.79 mL, 4.48 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate as a light yellow oil (0.71 g, 0.72 mmol, 48.2%). UPLC/ELSD: RT: 3.37 min. MS (ES): m/z (MH⁺) 986.4 for C₅₇H₁₀₀N₄O₉. ¹H NMR (300 MHz, CDCl₃) δ: ppm 5.39 (m, 2H), 4.64 (br. m, 1H), 3.27 (br. m, 11H), 2.38 (br. m, 6H), 1.86 (br. m, 13H), 1.46 (br. d, 32H), 1.15 (br. m, 11H), 1.03 (s, 5H), 0.94 (d, 3H, J=9 Hz), 0.88 (d, 5H, J=9 Hz), 0.70 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

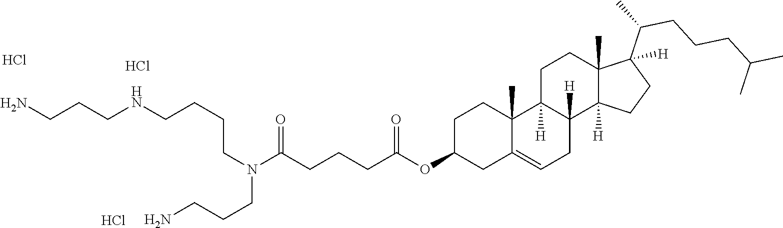

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate (0.71 g, 0.72 mmol) in 2-propanol (10 mL) stirring under nitrogen was added hydrochloric acid (5.5 M in 2-propanol, 1.44 mL, 7.20 mmol) dropwise. The mixture was heated to 45° C. and allowed to stir overnight. Then, the solution was cooled to room temperature, and acetonitrile (5 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride as a light purple solid (0.39 g, 0.47 mmol, 65.6%). UPLC/ELSD: RT: 1.68 min. MS (ES): m/z (MH⁺) 686.1 for C₄₂H₇₉Cl₃N₄O₃. ¹H NMR (300 MHz, CD₃OD) δ: ppm 5.40 (s, 1H), 4.90 (br. s, 9H), 4.55 (br. s, 1H), 3.33 (br. m, 12H), 2.32 (br. 6H), 2.16 (br. m, 2H), 2.05 (s, 5H), 1.91 (br. m, 10H), 1.54 (br. m, 7H), 1.39 (br. m, 4H), 1.17 (d, 8H, J=6 Hz), 1.06 (s, 5H), 0.97 (d, 4H, J=6 Hz), 0.90 (d, 6H, J=6 Hz), 0.73 (s, 3H).

### F. Compound SA59: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride

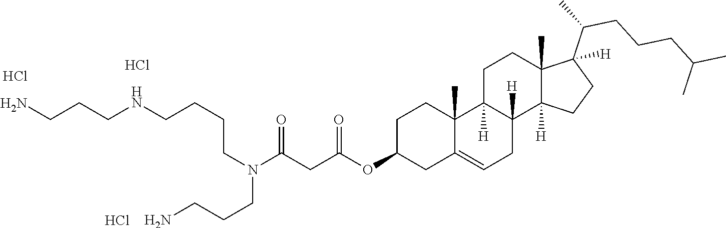

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaheptadecan-17-oate

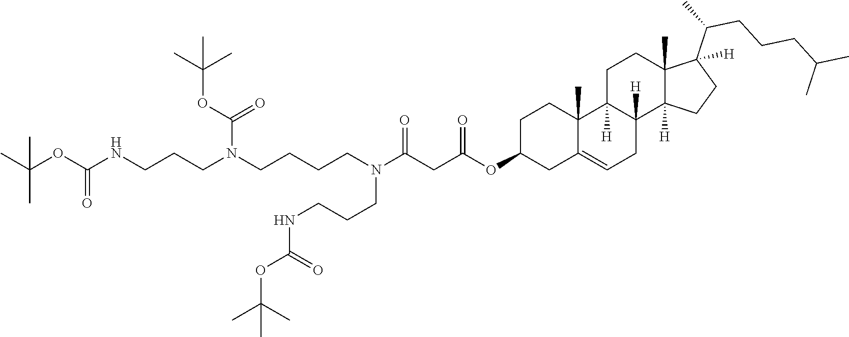

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-3-oxopropanoic acid (0.70 g, 1.47 mmol) in dichloromethane (20 mL) stirring under nitrogen was added tert-butyl (3-((tert-butoxycarbonyl)amino)propyl)(4-((3-((tert-butoxycarbonyl)amino)propyl)amino)butyl)carbamate (0.74 g, 1.47 mmol), dimethylaminopyridine (0.02 g, 0.15 mmol), and 1-ethyl-3-(3-dimethylaminopropyl)carbodiimide hydrochloride (0.43 g, 2.20 mmol). The resulting solution was cooled to 0° C., and diisopropylethylamine (0.78 mL, 4.40 mmol) was added dropwise. The mixture was allowed to gradually warm to room temperature and proceed overnight. Then, the solution was diluted with dichloromethane, washed with water (3×20 mL), dried over sodium sulfate, filtered, and concentrated to an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaheptadecan-17-oate as a light yellow oil (1.13 g, 1.18 mmol, 80.4%). UPLC/ELSD: RT: 3.29 min. MS (ES): m/z (MH⁺) 958.4 for C₅₅H₉₆N₄O₉. ¹H NMR (300 MHz, CDCl₃) δ: ppm 5.39 (m, 2H). 4.70 (br. m, 2H), 3.26 (br. m, 13H), 2.37 (d, 2H, J=6 Hz), 1.86 (br. m, 16H), 1.45 (br. s, 28H), 1.23 (br. m, 12H), 1.03 (s, 4H), 0.94 (d, 3H, J=6 Hz), 0.88 (d, 5H, J=6 Hz). 0.69 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride

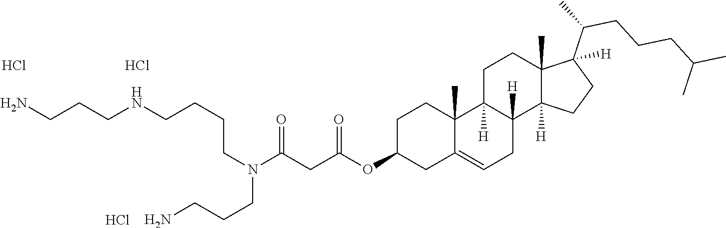

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaheptadecan-17-oate (1.13 g, 1.18 mmol) in 2-propanol (15 mL) stirring under nitrogen was added hydrochloric acid (5.5 M in 2-propanol, 2.36 mL, 11.79 mmol) dropwise. The mixture was heated to 40° C. and allowed to proceed overnight. Then, acetonitrile (5 mL) was added, and the solution was sonicated until all solid was displaced from the sides of the flask. After 30 minutes of stirring after sonication, the solid was filtered out by vacuum filtration and washed repeatedly with acetonitrile and dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-3-oxopropanoate trihydrochloride as a light purple solid (0.52 g, 0.64 mmol, 54.4%). UPLC/ELSD: RT: 1.57 min. MS (ES): m/z (MH⁺) 657.2 for C₄₀H₇₅N₄O₃. ¹H NMR (300 MHz, CD₃OD) δ: ppm 5.42 (m, 1H), 4.88 (br. s, 11H), 4.60 (m, 1H), 3.33 (br. m, 16H), 2.39 (d, 2H, J=3 Hz), 1.55 (br. m, 40H), 0.96 (d, 4H, J=6 Hz), 0.90 (d, 6H, J=6 Hz), 0.74 (s, 3H).

### G. Compound SA60: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

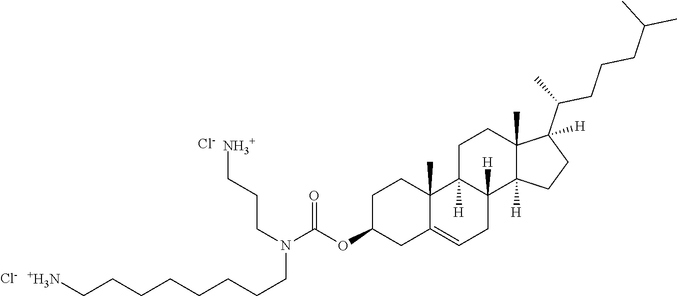

### Step 1: tert-Butyl N-{8-[(2-cyanoethyl)amino]octyl}carbamate

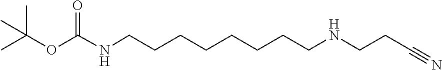 [SMILES: CC(C)(C)OC(=O)NCCCCCCCCNCCC#N]

To a foil-covered, stirred suspension of tert-butyl N-(8-aminooctyl)carbamate (2.50 g, 10.2 mmol) in water (100 mL) was added acrylonitrile (1.00 mL, 15.3 mmol). The suspension stirred at rt and was monitored by TLC. At 26 h, the reaction mixture was diluted with 5% aq. NaHCO₃ solution (200 mL) and then extracted with EtOAc (3×100 mL). The combined organics were washed with brine, dried over Na₂SO₄, and concentrated. The crude material was purified via silica gel chromatography (0-10% MeOH in DCM) to afford tert-butyl N-{8-[(2-cyanoethyl)amino]octyl}carbamate (1.833 g, 6.163 mmol, 60.2%) as a yellow oil. UPLC/ELSD: RT=0.28 min. MS (ES): m/z=197.9 [(M+H)—(CH₃)₂C═CH₂—CO₂]⁺ C₁₆H₃₁N₃O₂; ¹H NMR (300 MHz, CDCl₃): δ 4.50 (br. s, 1H), 3.09 (dt, 2H, 6.6, 6.5 Hz), 2.92 (t, 2H, J=6.6 Hz), 2.62 (t, 2H, J=7.1 Hz), 2.51 (t, 2H, J=6.7 Hz), 1.18-1.58 (m, 13H), 1.44 (s, 9H).

### Step 2: tert-Butyl N-{8-[benzyl(2-cyanoethyl)amino]octyl}carbamate

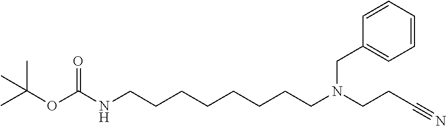 [SMILES: CC(C)(C)OC(=O)NCCCCCCCCN(CCC#N)Cc1ccccc1]

A mixture of tert-butyl N-{8-[(2-cyanoethyl)amino]octyl}carbamate (0.870 g, 2.92 mmol), potassium carbonate (0.808 g, 5.85 mmol), benzyl bromide (0.40 mL, 3.4 mmol), and potassium iodide (0.097 g, 0.58 mmol) in ACN (17.5 mL) was stirred at 65° C. The reaction was monitored by LCMS. At 3 h, the reaction mixture was cooled to rt, filtered through a pad of Celite, rinsed with MTBE, and concentrated. The residue was taken up in 5% aq. NaHCO₃ solution (50 mL) and then extracted with MTBE (3×30 mL). The combined organics were washed with brine, dried over Na₂SO₄, and concentrated. The crude material was purified via silica gel chromatography (0-40% EtOAc in hexanes) to afford tert-butyl N-{8-[benzyl(2-cyanoethyl)amino]octyl}carbamate (0.783 g, 2.02 mmol, 69.1%) as a clear oil. UPLC/ELSD: RT=0.44 min. MS (ES): m/z=331.9 [(M+H)—(CH₃)₂C═CH₂]⁺ for C₂₃H₃₇N₃O₂. ¹H NMR (300 MHz, CDCl₃): δ 7.21-7.39 (m, 5H), 4.49 (br. s, 1H), 3.60 (s, 2H), 3.09 (dt, 2H, J=6.5, 6.2 Hz), 2.78 (t, 2H, J=6.9 Hz), 2.48 (t, 2H, J=7.4 Hz), 2.39 (t, 2H, J=7.0 Hz), 1.38-1.54 (m, 4H), 1.44 (s, 9H), 1.19-1.36 (m, 8H).

### Step 3: tert-Butyl N-{3-[benzyl({8-[(tert-butoxycarbonyl)amino]octyl})amino]propyl}carbamate

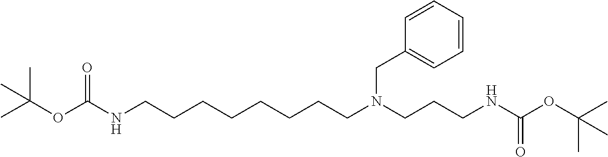 [SMILES: CC(C)(C)OC(=O)NCCCCCCCCN(CCCNC(=O)OC(C)(C)C)Cc1ccccc1]

To a stirred solution of tert-butyl N-{8-[benzyl(2-cyanoethyl)amino]octyl}carbamate (0.492 g, 1.27 mmol) in MeOH (8.8 mL) was added di-tert-butyl dicarbonate (0.693 g, 3.17 mmol) and nickel(II) chloride hexahydrate (0.030 g, 0.13 mmol). The reaction mixture was cooled to 0° C. in an ice bath and then NaBH₄ (0.336 g, 8.89 mmol) was added portionwise over 30 min to give a black suspension (CAUTION: VIGOROUS GAS EVOLUTION OCCURS DURING ADDITION). The reaction mixture stirred at rt and was monitored by LCMS. At 17.3 h, diethylenetriamine (0.15 mL, 1.4 mmol) was added dropwise, and the reaction mixture stirred at rt. After 30 min, additional diethylenetriamine (0.15 mL) was added. After 1.5 h, the reaction mixture was concentrated, taken up in 5% aq. NaHCO₃ solution and extracted with EtOAc (3×). The combined organics were washed with 5% aq. NaHCO₃ solution and brine, dried over Na₂SO₄, and concentrated. The crude material was purified via silica gel chromatography (20-65% EtOAc in hexanes) to afford tert-butyl N-{3-[benzyl({8-[(tert-butoxycarbonyl)amino]octyl})amino]propyl}carbamate (0.512 g, 1.04 mmol, 82.0%) as a clear oil. UPLC/ELSD: RT=0.92 min. MS (ES): m/z=492.5 [M+H]⁺ for C₂₈H₄₉N₃O₄; ¹H NMR (300 MHz, CDCl₃): δ 7.19-7.35 (m, 5H), 5.52 (br. s, 1H), 4.49 (br. s, 1H), 3.51 (s, 2H), 3.00-3.22 (m, 4H), 2.46 (t, 2H, J=6.2 Hz), 2.36 (t, 2H, J=7.4 Hz), 1.56-1.68 (m, 2H), 1.36-1.55 (m, 22H), 1.18-1.33 (m, 8H).

### Step 4: tert-Butyl N-[3-({8-[(tert-butoxycarbonyl)amino]octyl}amino)propyl]carbamate

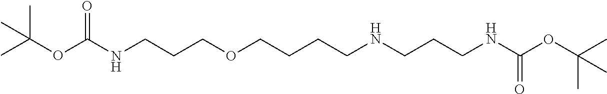 [SMILES: CC(C)(C)OC(=O)NCCCNCCCCOCCCNC(=O)OC(C)(C)C]

A solution of tert-butyl N-{3-[benzyl({8-[(tert-butoxycarbonyl)amino]octyl})amino]propyl}carbamate (0.496 g, 1.01 mmol) and 10% Pd/C (0.429 g, 0.202 mmol) in ethanol (10 mL) was stirred under a balloon of H₂. The reaction was monitored by TLC. At 3 h, the reaction mixture was diluted with EtOAc (20 mL), filtered through a pad of Celite, and rinsed with EtOAc. The filtrate was concentrated, taken up in EtOAc, and filtered using a 0.45 μm syringe filter. Filtered organics were concentrated to afford tert-butyl N-[3-({8-[(tert-butoxycarbonyl)amino]octyl}amino)propyl]carbamate (0.323 g, 0.805 mmol, 79.8%) as an off-white solid). UPLC/ELSD: RT=0.59 min. MS (ES): m/z=402.0 [M+H]⁺ for C₂₁H₄₃N₃O₄; ¹H NMR (300 MHz, CDCl₃): δ 5.17 (br. s, 1H), 4.50 (br. s, 1H), 3.20 (dt, 2H, J=6.0, 6.0 Hz), 3.09 (dt, 2H, J=6.5, 6.4 Hz), 2.67 (t, 2H, J=6.6 Hz), 2.58 (t, 2H, J=7.1 Hz), 1.89 (br. s, 1H), 1.59-1.74 (m, 2H), 1.37-1.55 (m, 22H), 1.21-1.37 (m, 8H).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate

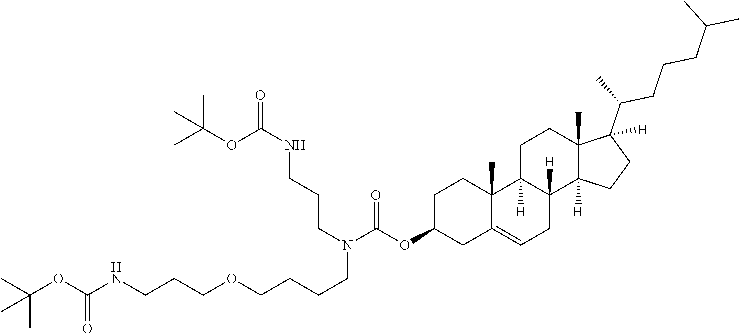

Cholesterol 4-nitrophenyl carbonate (0.300 g, 0.544 mmol), tert-butyl N-[3-({8-[(tert-butoxycarbonyl)amino]octyl}amino)propyl]carbamate (0.240 g, 0.598 mmol), and triethylamine (0.12 mL, 0.85 mmol) were combined in CHCl₃ (4.8 mL). The reaction mixture stirred at 50° C. and was monitored by TLC. At 20.25 h, tert-butyl N-[3-({8-[(tert-butoxycarbonyl)amino]octyl}amino)propyl]carbamate (77 mg) and triethylamine (0.04 mL) were added. The reaction mixture stirred at 60° C. At 95 h, the reaction mixture was cooled to rt, diluted with DCM (20 mL), and washed with water (25 mL).

The aqueous layer was extracted with DCM (2×20 mL). The combined organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.398 g, 0.489 mmol, 89.9%) as a clear oil. UPLC/ELSD: RT=3.47 min. MS (ES): m/z=836.5 [M+Na]⁺ for C₄₉H₈₇N₃O₆; ¹H NMR (300 MHz, CDCl₃): δ 5.22-5.43 (m, 2H), 4.40-4.84 (m, 2H), 3.00-3.39 (br. m, 8H), 2.21-2.44 (m, 2H), 1.73-2.07 (br. m, 5H), 0.93-1.71 (br. m, 53H), 1.02 (s, 3H), 0.91 (d, 3H, J=6.4 Hz), 0.86 (d, 3H, J=6.6 Hz), 0.86 (d, 3H, J=6.6 Hz), 0.67 (s, 3H).

### Step 6: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

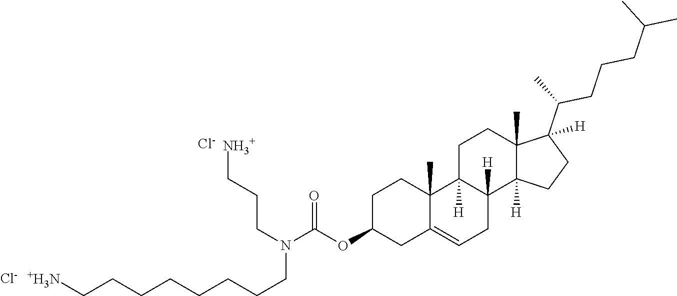

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.395 g, 0.485 mmol) in iPrOH (2.5 mL) was added 5-6 N HCl in iPrOH (0.7 mL). The reaction mixture stirred at 40° C. and was monitored by LCMS. At 17.5 h, the reaction mixture was cooled to rt. ACN (5 mL) was added, the suspension was stirred for 15 min, and the solids were collected by vacuum filtration rinsing with 2:1 ACN:iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride (0.249 g, 0.356 mmol, 73.4%) as a white solid. UPLC/ELSD: RT=1.97 min. MS (ES): m/z=614.4 [M+H]⁺ for C₃₉H₇₃Cl₂N₃O₂; ¹H NMR (300 MHz, CDCl₃): δ 8.00-8.64 (br. m, 6H), 5.33-5.44 (m, 1H), 4.39-4.56 (m, 1H), 2.93-3.54 (br. m, 8H), 2.20-2.43 (m, 2H), 1.69-2.16 (br. m, 10H), 0.93-1.66 (br. m, 30H), 1.02 (s, 3H), 0.91 (d, 3H, J=6.3 Hz), 0.87 (d, 3H, J=6.5 Hz), 0.86 (d, 3H, J=6.5 Hz), 0.68 (s, 3H).

### H. Compound SA61: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride

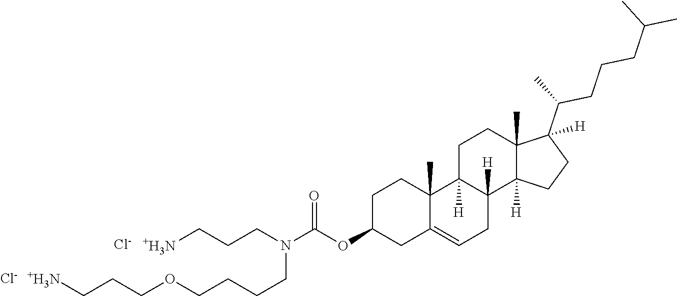

### Step 1: 3-(4-Hydroxybutoxy)propanenitrile

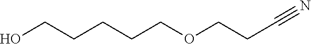 [SMILES: N#CCCOCCCCCO]

To a solution of 1,4-butanediol (8.0 mL, 91 mmol) and benzyltrimethylammonium hydroxide (0.20 mL, 1.3 mmol, 40 wt % in water) was added acrylonitrile (3.0 mL, 46 mmol). The reaction mixture stirred at rt while covered in foil and was monitored by TLC. At 2 h, the reaction mixture was diluted with water (150 mL) and extracted with 1:1 hexanes/MTBE (50 mL) and EtOAc (3×50 mL). The combined organics were washed with water and brine, dried over MgSO₄, and concentrated. The crude material was purified via silica gel chromatography (50-100% EtOAc in hexanes) to afford 3-(4-hydroxybutoxy)propanenitrile (1.374 g, 9.596 mmol, 21.0%) as a yellow oil. UPLC/ELSD: RT=0.20 min. ¹H NMR (300 MHz, CDCl₃): δ 3.67 (t, 2H, J=6.0 Hz), 3.66 (t, 2H, J=6.4 Hz), 3.50-3.57 (m, 2H), 2.60 (t, 2H, 6.4 Hz), 1.60-1.76 (m, 5H).

### Step 2: 4-(2-Cyanoethoxy)butyl methanesulfonate

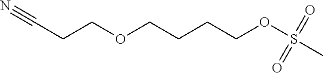

A stirred solution of 3-(4-hydroxybutoxy)propanenitrile (1.00 g, 6.98 mmol) and triethylamine (1.5 mL, 11 mmol) in DCM (10 mL) was cooled to 0° C. in an ice bath, and then methanesulfonyl chloride (0.60 mL, 7.8 mmol) was added dropwise. The reaction was monitored by TLC. The reaction mixture was allowed to slowly come to rt. At 2 h, the reaction mixture was cooled to 0° C. in an ice bath, and additional methanesulfonyl chloride (0.06 mL) was added. At 2 h 10 min, water (10 mL) was added, and the reaction mixture stirred at rt for 5 min. After this time, a 5% aq. NaHCO₃ solution (50 mL) was added, and then the reaction mixture was extracted with DCM (3×30 mL). The combined organics were washed with water and brine, dried over MgSO₄, and concentrated to afford 4-(2-cyanoethoxy)butyl methanesulfonate (1.556 g, 7.032 mmol, quant.) as a yellow oil. The material was carried forward without further purification into the next step. ¹H NMR (300 MHz, CDCl₃): δ 4.28 (t, 2H, J=6.4 Hz), 3.64 (t, 2H, J=6.2 Hz), 3.54 (t, 2H, J=5.9 Hz), 3.01 (s, 3H), 2.59 (t, 2H, J=6.2 Hz), 1.81-1.93 (m, 2H), 1.66-1.78 (m, 2H).

### Step 3: tert-Butyl N-(3-{[4-(2-cyanoethoxy)butyl]amino}propyl)carbamate

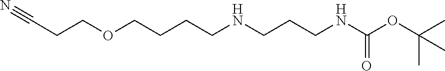 [SMILES: CC(C)(C)OC(=O)NCCCNCCCCOCCC#N]

A solution of tert-butyl N-(3-aminopropyl)carbamate (4.272 g, 24.52 mmol), 4-(2-cyanoethoxy)butyl methanesulfonate (1.550 g, 7.005 mmol), and EtOH (16 mL) was stirred at 65° C. The reaction was monitored by TLC. At 4 h, the reaction mixture was cooled to rt. At 21.5 h, the reaction mixture was concentrated and then taken up in a mixture of EtOAc (75 mL) and water (75 mL). The layers were separated, and the aqueous was extracted with EtOAc (50 mL). The combined organics were washed with water (3×) and brine, dried over Na₂SO₄, and concentrated. The crude material was purified via silica gel chromatography (0-20% (5% conc. aq. NH₄OH in MeOH) in DCM) to afford tert-butyl N-(3-{[4-(2-cyanoethoxy)butyl]amino}propyl)carbamate (1.396 g, 4.662 mmol, 66.6%) as a yellow oil. UPLC/ELSD: RT=0.22 min. MS (ES): m/z=243.8 [(M+H)−t-Bu]⁺ for C₁₅H₂₉N₃O₃; ¹H NMR (300 MHz, CDCl₃): δ 5.19 (br. s, 1H), 3.64 (t, 2H, J=6.3 Hz), 3.50 (t, 2H, J=6.0 Hz), 3.20 (dt, 2H, J=6.2, 5.9 Hz), 2.67 (t, 2H, J=6.6 Hz), 2.61 (t, 2H, J=6.7 Hz), 2.59 (t, 2H, J=6.4 Hz), 1.48-1.70 (m, 6H), 1.44 (s, 9H), 1.10 (br. s, 1H).

### Step 4: tert-Butyl N-(3-{benzyl[4-(2-cyanoethoxy)butyl]amino}propyl)carbamate

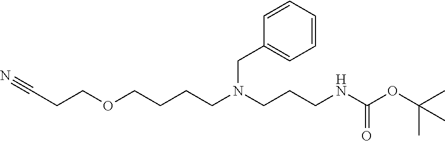 [SMILES: CC(C)(C)OC(=O)NCCCN(CCCCOCCC#N)Cc1ccccc1]

To a mixture of tert-butyl N-(3-{[4-(2-cyanoethoxy)butyl]amino}propyl)carbamate (1.380 g, 4.609 mmol), potassium carbonate (1.274 g, 9.218 mmol), and potassium iodide (0.150 g, 0.904 mmol) in ACN (20 mL) was added benzyl bromide (0.63 mL, 5.3 mmol). The reaction mixture stirred at 65° C. and was monitored by TLC. At 2.5 h, the reaction mixture was cooled to rt and filtered through a pad of Celite rinsing with ACN, and the filtrate was concentrated. The residue was taken up in 5% aq. NaHCO₃ solution (ca. 50 mL), then extracted with MTBE (2×25 mL), and EtOAc (25 mL). The combined organics were washed with brine, dried over Na₂SO₄, and concentrated. The crude material was purified via silica gel chromatography (30-70% EtOAc in hexanes) to afford tert-butyl N-(3-{benzyl[4-(2-cyanoethoxy)butyl]amino}propyl)carbamate (1.372 g, 3.522 mmol, 76.4%) as a yellow oil. UPLC/ELSD: RT=0.29 min. MS (ES): m/z=390.0 [M+H]⁺ for C₂₂H₃₅N₃O₃. ¹H NMR (300 MHz, CDCl₃): δ 7.20-7.39 (m, 5H), 5.41 (br. s, 1H), 3.60 (t, 2H, J=6.4 Hz), 3.51 (s, 2H), 3.38-3.47 (m, 2H), 3.15 (dt, 2H, J=5.8, 5.6 Hz), 2.56 (t, 2H, J=6.4 Hz), 2.47 (t, 2H, J=6.3 Hz), 2.35-2.43 (m, 2H), 1.51-1.69 (m, 6H), 1.44 (s, 9H).

### Step 5: tert-Butyl N-{3-[benzyl(4-{3-[(tert-butoxycarbonyl)amino]propoxy}butyl)amino]propyl}carbamate

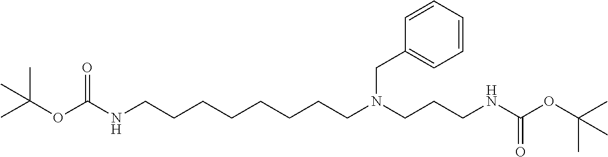 [SMILES: CC(C)(C)OC(=O)NCCCCCCCCN(CCCNC(=O)OC(C)(C)C)Cc1ccccc1]

To a stirred solution of tert-butyl N-(3-{benzyl[4-(2-cyanoethoxy)butyl]amino}propyl)carbamate (1.357 g, 3.484 mmol) in MeOH (23 mL) was added di-tert-butyl dicarbonate (1.901 g, 8.709 mmol) and nickel(II) chloride hexahydrate (0.083 g, 0.35 mmol). The reaction mixture was cooled to 0° C. in an ice bath, and then NaBH₄ (0.923 g, 24.4 mmol) was added portion wise over 40 min (CAUTION: VIGOROUS GAS EVOLUTION OCCURS DURING ADDITION). The reaction mixture stirred at rt and was monitored by LCMS. At 17.25 h, the reaction mixture was cooled to 0° C. in an ice bath, and then NaBH₄ (500 mg) was added portion wise over 30 min. The reaction mixture stirred at rt. At 18.5 h, the reaction mixture was cooled to 0° C. in an ice bath, and then NaBH₄ (100 mg) was added. The reaction mixture stirred at 0° C. At 19.5 h, NaBH₄ (101 mg) was added. At 20.5 h, NaBH₄ (102 mg) was added. At 21.5 h, Boc₂O (850 mg) and NaBH₄ (103 mg) were added. The reaction mixture was allowed to slowly come to rt. At 40.5 h, diethylenetriamine (0.55 mL, 5.1 mmol) was added dropwise, and the reaction mixture stirred at rt for 1 h. After this time, the reaction mixture was concentrated, taken up in 5% aq. NaHCO₃ solution, and extracted with DCM (3×). The biphasic mixture was concentrated to remove volatile organics, and then the mixture was extracted with MTBE (3×). The combined organics were washed with brine, dried over Na₂SO₄, and concentrated. The crude material was purified via silica gel chromatography (0-7% (5% conc. aq. NH₄OH in MeOH) in DCM) to afford tert-butyl N-{3-[benzyl(4-{3-[(tert-butoxycarbonyl)amino]propoxy}butyl)amino]propyl}carbamate (0.836 g, 1.69 mmol, 48.6%) as a light yellow oil. UPLC/ELSD: RT=0.65 min. MS (ES): m/z=494.5 [M+H]⁺ for C₂₇H₄₇N₃O₅; ¹H NMR (300 MHz, CDCl₃): δ 7.19-7.38 (m, 5H), 5.43 (br. s, 1H), 4.87 (br. s, 1H), 3.51 (s, 2H), 3.43 (t, 2H, J=5.9 Hz), 3.31-3.39 (m, 2H), 3.06-3.26 (m, 4H), 2.46 (t, 2H, J=6.2 Hz), 2.35-2.43 (m, 2H), 1.49-1.79 (br. m, 8H), 1.44 (s, 18H).

### Step 6: tert-Butyl N-{3-[4-({3-[(tert-butoxycarbonyl)amino]propyl}amino)butoxy]propyl}carbamate

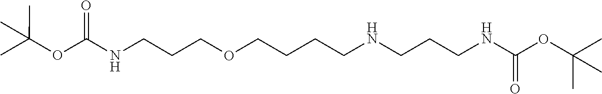 [SMILES: CC(C)(C)OC(=O)NCCCNCCCCOCCCNC(=O)OC(C)(C)C]

A solution of tert-butyl N-{3-[benzyl(4-{3-[(tert-butoxycarbonyl)amino]propoxy}butyl)amino]propyl}carbamate (0.825 g, 1.67 mmol) and 10% Pd/C (0.711 g, 0.334 mmol) in EtOH (10 mL) was stirred under a balloon of H₂. The reaction was monitored by TLC. At 18 h, the reaction mixture was diluted with EtOAc (40 mL) and then filtered through a pad of Celite rinsing with EtOAc. The filtrate was concentrated, taken up in EtOAc, and filtered using a 0.45 μm syringe filter. Filtered organics were concentrated to afford tert-butyl N-{3-[4-({3-[(tert-butoxycarbonyl)amino]propyl}amino)butoxy]propyl}carbamate (0.636 g, 1.58 mmol, 94.3%) as a yellow oil. UPLC/ELSD: RT=0.40 min. MS (ES): m/z=404.5 [M+H]⁺ for C₂₀H₄₁N₃O₅; ¹H NMR (300 MHz, CDCl₃): δ 5.20 (br. s, 1H), 4.91 (br. s, 1H), 3.47 (t, 2H, J=5.9 Hz), 3.41 (t, 2H, J=6.1 Hz), 3.13-3.23 (m, 4H), 2.67 (t, 2H, J=6.6 Hz), 2.61 (t, 2H, J=6.6 Hz), 1.48-1.80 (br. m, 9H), 1.44 (s, 18H).

### Step 7: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate

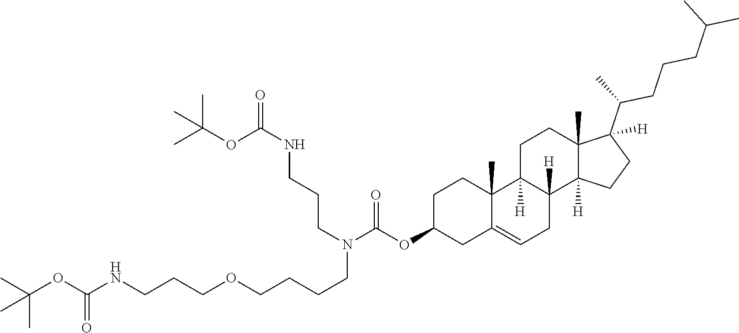

A solution of cholesterol 4-nitrophenyl carbonate (0.663 g, 1.20 mmol), tert-butyl N-{3-[4-({3-[(tert-butoxycarbonyl)amino]propyl}amino)butoxy]propyl}carbamate (0.630 g, 1.56 mmol), and triethylamine (0.50 mL, 3.6 mmol) in PhMe (10 mL) was stirred at 90° C. The reaction was monitored by LCMS. At 18 h, the reaction mixture was cooled to rt and concentrated. The residue was dissolved in DCM (50 mL) and then washed with water (3×30 mL). The organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate (0.825 g, 1.01 mmol, 84.2%) as a tacky white foam. UPLC/ELSD: RT=3.39 min. MS (ES): m/z=839.2 [M+Na]⁺ for C₄₈H₈₅N₃O₇; ¹H NMR (300 MHz, CDCl₃): δ 5.34-5.43 (m, 1H), 5.30 (br. s, 1H), 4.73-5.00 (m, 1H), 4.41-4.59 (m, 1H), 3.46 (t, 2H, J=5.9 Hz), 3.41 (t, 2H, J=5.9 Hz), 3.00-3.36 (br. m, 8H), 2.20-2.43 (m, 2H), 0.93-2.09 (br. m, 34H), 1.43 (s, 18H), 1.02 (s, 3H), 0.91 (d, 3H, J=6.4 Hz), 0.86 (d, 6H, J=6.5 Hz), 0.67 (s, 3H).

### Step 8: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride

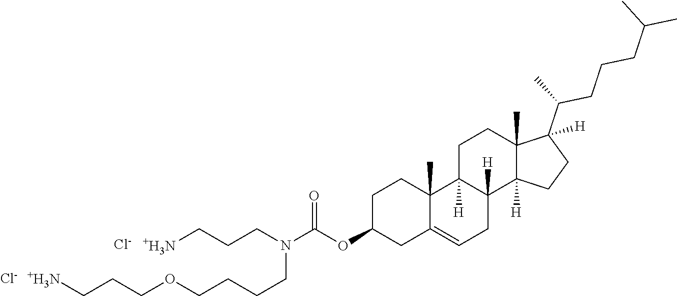

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-((tert-butoxycarbonyl)amino)propoxy)butyl)(3-((tert-butoxycarbonyl)amino)propyl)carbamate (0.809 g, 0.991 mmol) in iPrOH (6.0 mL) was added 5-6 N HCl in iPrOH (1.4 mL). The reaction mixture stirred at 40° C. and was monitored by LCMS. At 15.5 h, the reaction mixture was cooled to rt. ACN (18 mL) was added to the reaction mixture, and the suspension stirred at rt for 10 min. After this time, solids were collected by vacuum filtration and rinsed with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate dihydrochloride (0.609 g, 0.828 mmol, 83.6%) as a white solid. UPLC/ELSD: RT=2.00 min. MS (ES): m/z=617.0 [M+H]⁺ for C₃₈H₆₉N₃O₃; ¹H NMR (300 MHz, CDCl₃): δ 8.51-8.82 (m, 3H), 8.05 (br. s, 3H), 5.33-5.42 (m, 1H), 4.42-4.57 (m, 1H), 3.63 (t, 2H, J=5.4 Hz), 2.97-3.58 (br. m, 10H), 2.19-2.43 (m, 2H), 0.93-2.13 (br. m, 34H), 1.02 (s, 3H), 0.91 (d, 3H, J=6.4 Hz), 0.86 (d, 3H, J=6.5 Hz), 0.86 (d, 3H, J=6.5 Hz), 0.67 (s, 3H).

### I. Compound SA62: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride

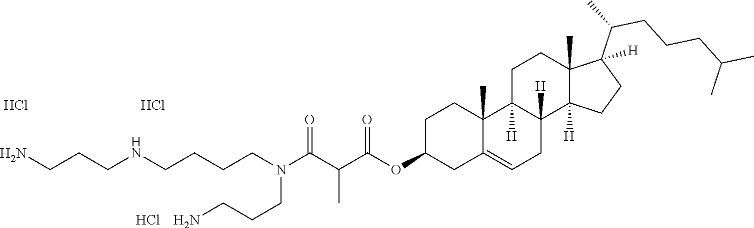

### Step 1: 1-(tert-Butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate

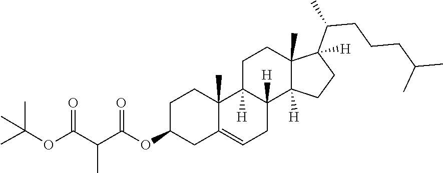

To a solution of cholesterol (1.85 g, 4.69 mmol) and 3-(tert-butoxy)-2-methyl-3-oxopropanoic acid (0.96 mL, 5.63 mmol) in dichloromethane (50 mL) stirring under nitrogen was added 1-ethyl-3-(3-dimethylaminopropyl)carbodiimide hydrochloride (1.36 g, 7.03 mmol). Then the reaction mixture was cooled to 0° C., and diisoproylethylamine (2.48 mL, 14.07 mmol) was added dropwise over 20 minutes. The resulting mixture was allowed to gradually warm to room temperature and proceed overnight. The mixture was then diluted with dichloromethane to 150 mL, washed with water (1×70 mL), saturated aqueous sodium bicarbonate (2×70 mL), and brine (1×70 mL), dried over sodium sulfate, filtered, and concentrated in vacuo to give a yellow oil. The oil was taken up in dichloromethane and purified on silica with a 0-25% ethyl acetate gradient in hexanes to give 1-(tert-butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate as an oil (1.62 g, 2.99 mmol, 63.7%). UPLC/ELSD: RT: 3.41 min. MS (ES): m/z (MH⁺) 543.8 for C₃₅H₅₈O₄. ¹H NMR (300 MHz, CDCl₃) δ: ppm 5.31 (m, 1H), 4.58 (br. m, 1H), 3.21 (q, 1H, J=6 Hz), 2.27 (d, 2H, J=9 Hz), 1.87 (br. m, 6H), 1.50 (br. m, 6H), 1.39 (s, 12H), 1.28 (br. m, 12H), 1.07 (br. m, 8H), 0.95 (s, 4H), 0.91 (d, 2H, J=6 Hz), 0.86 (d, 4H, J=6 Hz), 0.80 (d, 8H, J=6 Hz), 0.61 (s, 3H).

### Step 2: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-JH-cyclopenta[a]phenanthren-3-yl)oxy)-2-methyl-3-oxopropanoic acid

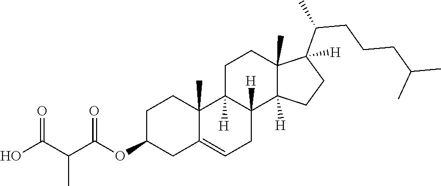

A solution of 1-(tert-butyl) 3-((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl) 2-methylmalonate (1.62 g, 2.99 mmol) in dichloromethane (50 mL) was cooled to 0° C. To this solution was added trifluoroacetic acid (3.43 mL, 44.79 mmol) dropwise over 20 minutes. The reaction mixture was allowed to gradually warm to room temperature and proceed for 5 hours, slowly turning a light pink. The crude reaction mixture was concentrated in vacuo to a pink solid, taken up in DCM, and purified on silica with a 0-40% ethyl acetate gradient in hexanes to give 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-methyl-3-oxopropanoic acid as a white solid (1.05 g, 2.16 mmol, 72.2%). UPLC/ELSD: RT: 3.02 min. MS (ES): m/z (MH⁺) 487.7 for C₃₁H₅₀O₄. ¹H NMR (300 MHz, CDCl₃) δ: ppm 11.03 (br. s, 1H), 5.40 (br. d, 1H), 4.72 (br. m, 1H), 3.49 (q, 1H, J=6 Hz), 2.38 (d, 2H, J=9 Hz), 2.01 (br. m, 5H), 1.61 (br. m, 5H), 1.50 (d, 5H, J=6 Hz), 1.27 (br. m, 12H), 1.04 (s, 5H), 0.95 (d, 4H, J=6 Hz), 0.90 (d, 6H, J=6 Hz), 0.70 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate

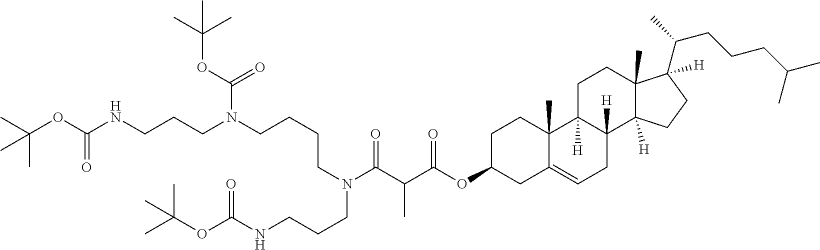

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-methyl-3-oxopropanoic acid (0.50 g, 1.02 mmol) in dichloromethane (10 mL) stirring under nitrogen was added tert-butyl (3-((tert-butoxycarbonyl)amino)propyl)(4-((3-((tert-butoxycarbonyl)amino)propyl)amino)butyl)carbamate (0.72 g, 1.42 mmol), dimethylaminopyridine (0.01 g, 0.10 mmol), and 1-ethyl-3-(3-dimethylaminopropyl)carbodiimide hydrochloride (0.30 g, 1.53 mmol). The resulting solution was cooled to 0° C. and diisopropylethylamine (0.54 mL, 3.05 mmol) was added dropwise. The mixture was allowed to gradually warm to room temperature and proceed overnight. Then, the solution was diluted with dichloromethane, washed with water (3×10 mL), dried over sodium sulfate, filtered, and concentrated to give an oil. The oil was taken up in DCM and purified on silica with a 0-60% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate as a light yellow oil (0.18 g, 0.19 mmol, 18.2%). UPLC/ELSD: RT: 3.26 min. MS (ES): m/z (MH⁺) 972.4 for C₅₆H₉₈N₄O₉. ¹H NMR (300 MHz, CDCl₃) δ: ppm 5.36 (br. s, 1H), 4.58 (br. m, 1H), 4.10 (q, 1H, J=6 Hz), 3.36 (br. m, 13H), 2.26 (br. m, 3H), 2.01 (s, 4H), 1.80 (br. m, 10H), 1.43 (br. m, 47H), 1.23 (t, 4H, J=9 Hz), 1.08 (br. m, 7H), 0.97 (s, 8H), 0.90 (d, 4H, J=9 Hz), 0.84 (d, 6H, J=6 Hz), 0.65 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride

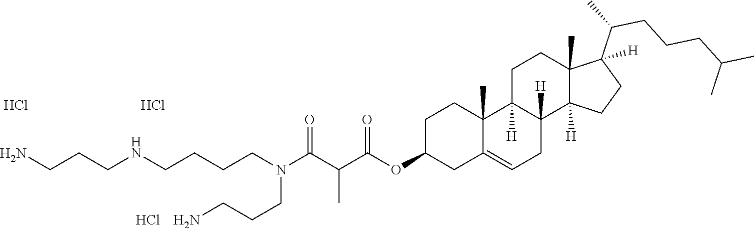

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,16-trimethyl-4,15-dioxo-3-oxa-5,9,14-triazaheptadecan-17-oate (0.18 g, 0.19 mmol) in isopropanol (10 mL) stirring under nitrogen was added hydrochloric acid (5.5 M in isopropanol, 0.37 mL, 1.85 mmol) dropwise. The mixture was heated to 45° C. and allowed to stir overnight. Then, the solution was cooled to room temperature and acetonitrile (5 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-((3-aminopropyl)(4-((3-aminopropyl)amino)butyl)amino)-2-methyl-3-oxopropanoate trihydrochloride as a white solid (0.06 g, 0.07 mmol, 38.7%). UPLC/ELSD: RT: 1.70 min. MS (ES): m/z (MH⁺) 672.1 for C₄₁H₇₇Cl₃N₄O₃. ¹H NMR (300 MHz, CD₃OD) δ: ppm 5.41 (s, 1H), 4.88 (br. m, 10H), 4.58 (br. m, 1H), 3.92 (br. m, 1H), 3.56 (br. m, 4H), 3.33 (s, 3H), 3.10 (br. m, 8H), 2.34 (br. m, 2H), 2.05 (br. m, 15H), 1.54 (br. m, 8H), 1.38 (br. m, 8H), 1.17 (d, 9H, J=6 Hz), 1.06 (s, 6H), 0.97 (d, 4H, J=6 Hz), 0.90 (d, 6H, J=6 Hz), 0.73 (s, 3H).

### J. Compound SA63: (3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate

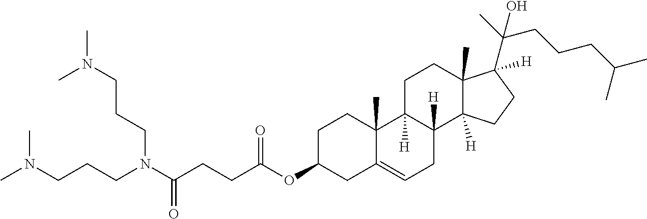

### Step 1: (3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-ol

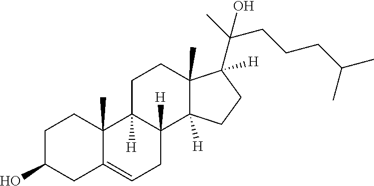

A mixture of magnesium turnings (2.21 g, 90.87 mmol) and iodine (1.54 g, 6.06 mmol) were purged twice with vacuum and nitrogen and then held under nitrogen. To this mixture was added dry tetrahydrofuran (50 mL) and set stirring under nitrogen. To this mixture was added 1-bromo-4-methylpentane (8.82 mL, 60.58 mmol) dropwise over 10 minutes, and then the reaction was allowed to proceed for one hour at room temperature. Following, the reaction mixture was refluxed at 66° C. for 3 hours, during which the grey reaction slurry turned to a clear colorless solution with some undissolved magnesium. The reaction was then cooled to 0° C. upon which the solution became cloudy again. At 0° C., a solution of pregnenolone (5.75 g, 18.17 mmol) in dry tetrahydrofuran (25 mL) was added dropwise over an hour, during which the reaction mixture solidified. Following, the solution was warmed to room temperature, an additional 50 mL tetrahydrofuran was added, and the reaction was allowed to continue at 30° C. overnight, during which the solidified mixture broke into smaller pieces stirring in the added solvent. The reaction was quenched the following day with saturated aqueous ammonium chloride (50 mL) and then diluted with 100 mL ethyl acetate. The aqueous layer was separated, and extracted again with 100 mL ethyl acetate. Then the organic layers were combined, washed with water (1×100 mL) and brine (1×100 mL), dried over sodium sulfate, filtered, and concentrated to dryness. The resulting residue was taken up in DCM and purified on silica with a 0-50% ethyl acetate gradient in hexanes to give (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-ol as a white solid (2.68 g, 6.64 mmol, 36.6%). UPLC/ELSD: RT: 2.13 min. MS (ES): m/z (MH⁺) 403.7 for C₂₇H₄₆O₂. ¹H NMR (300 MHz, CDCl₃) δ: ppm 5.36 (br. d, 1H, J=6 Hz), 3.54 (br. m, 1H), 2.29 (br. m, 2H), 2.06 (br. m, 2H), 1.85 (br. m, 16H), 1.29 (s, 6H), 1.17 (br. m, 6H), 1.03 (s, 6H), 0.88 (d, 10H, J=6 Hz).

### Step 2: 4-(((3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid

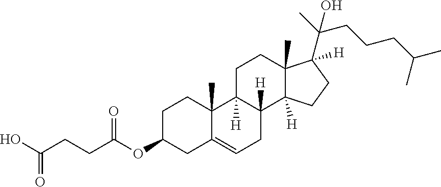

To a solution of (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-ol (0.50 g, 1.24 mmol) in dichloromethane (10 mL) stirring under nitrogen was added succinic anhydride (0.12 g, 1.24 mmol). Then pyridine (0.17 mL, 1.24 mmol) was added dropwise at room temperature, and the mixture was refluxed at 40° C. overnight upon which all solid went into solution. The following day, TLC revealed incomplete conversion, and dimethylaminopyridine (0.05 g, 0.41 mmol) and succinic anhydride (0.03 g, 0.25 mmol) were added before the reaction mixture was allowed to reflux overnight again at 40° C. The following morning, the mixture was concentrated in vacuo to a yellow oil. The yellow oil was taken up in dichloromethane and purified on silica with a 0-30% ethyl acetate gradient in hexanes to give 4-(((3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid as a white solid (0.27 g, 0.54 mmol, 43.3%). UPLC/ELSD: RT: 2.20 min. MS (ES): m/z (MH⁺) 503.8 for C₃₁H₅₀O₅. ¹H NMR (300 MHz, CDCl₃) δ: ppm 6.60 (br. s, 1H), 5.38 (br. s, 1H), 4.64 (br. m, 1H), 4.13 (q, 1H, J=6 Hz), 2.66 (dd, 4H, J=6 Hz), 2.33 (d, 2H, J=6 Hz), 2.05 (br. m, 2H), 1.84 (br. m, 3H), 1.51 (br. m, 12H), 1.28 (br. m, 8H), 1.13 (br. m, 5H), 1.02 (s, 4H), 0.86 (d, 10H, J=6 Hz).

### Step 3: (3S,8S,9S,10R,13S,14S,17S)-17-(2-Hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate

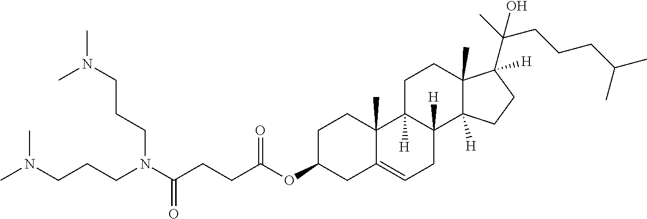

To a solution of 4-(((3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl)oxy)butanoic acid (0.35 g, 0.68 mmol) in dichloromethane (10 mL) stirring under nitrogen was added tetramethyldipropylenetriamine (0.24 mL, 1.02 mmol), dimethylaminopyridine (0.01 g, 0.07 mmol), and 1-ethyl-3-(3-dimethylaminopropyl)carbodiimide hydrochloride (0.20 g, 1.02 mmol). The resulting solution was cooled to 0° C., and diisopropylethylamine (0.36 mL, 2.04 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to give an oil. The oil was taken up in DCM and purified on silica with a 0-60% (80:19:1 DCM/MeOH/NH₄OH) gradient in DCM to give (3S,8S,9S,10R,13S,14S,17S)-17-(2-hydroxy-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-(dimethylamino)propyl)amino)-4-oxobutanoate as a light yellow oil (0.07 g, 0.09 mmol, 13.6%). UPLC/ELSD: RT: 1.25 min. MS (ES): m/z (MH⁺) 673.0 for C₄₁H₇₃N₃O₄. ¹H NMR (300 MHz, CDCl₃) δ: ppm 5.37 (br. s, 1H), 4.64 (br. m, 1H), 3.35 (br. t, 4H, J=9 Hz), 2.64 (s, 4H), 2.28 (br. m, 6H), 2.22 (s, 12H), 1.83 (br. m, 4H), 1.60 (br. m, 15H), 1.28 (br. s, 7H), 1.13 (br. m, 5H), 1.02 (s, 4H), 0.89 (d, 9H, J=6 Hz).

### K. Compound SA64: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-(dimethylamino)octyl)(3-(dimethylamino)propyl)carbamate

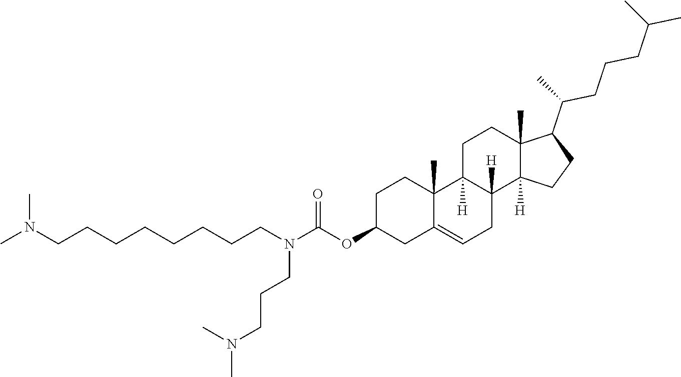

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-aminooctyl)(3-aminopropyl)carbamate (105 mg, 0.15 mmol) and sodium acetate trihydrate (208 mg, 1.53 mmol) in 6 mL methanol at room temperature was added formaldehyde (0.12 mL, 37 wt % in water, 1.53 mmol) and sodium cyanoborohydride (96.1 mg, 1.53 mmol). The solution was stirred at room temperature for 16 hours, after which no starting aminosterol remained by LCMS. The mixture was diluted with 2 M aqueous NaOH solution and extracted three times with DCM. The organics were combined, washed once with brine, dried (MgSO₄), filtered, and concentrated. The residue was purified by silica gel chromatography (0-50% (mixture of 1% concentrated aq. NH₄OH and 20% MeOH in DCM) in DCM) to give 3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-(dimethylamino)octyl)(3-(dimethylamino)propyl)carbamate (63.2 mg, 0.091 mmol, 60%) as a colorless oil. UPLC/ELSD: RT=2.14 min. MS (ES): m/z (MH⁺) 671.2 for C₄₃H₈₀N₃O₂. ¹H NMR (300 MHz, CDCl₃) δ: ppm 5.35 (d, 1H, J=5 Hz); 4.48 (septet, 1H, J=5 Hz); 3.19 (s, 4H); 2.39-2.27 (m, 12H); 2.25 (s, 6H); 2.22 (s, 6H); 2.03-1.63 (m, 8H); 1.58-1.04 (m, 23H); 1.00 (s, 6H); 0.90 (d, 3H, J=6 Hz); 0.86 (d, 3H, J=1 Hz); 0.84 (d, 3H, J=1 Hz); 0.66 (s, 3H).

### L. Compound SA65: ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

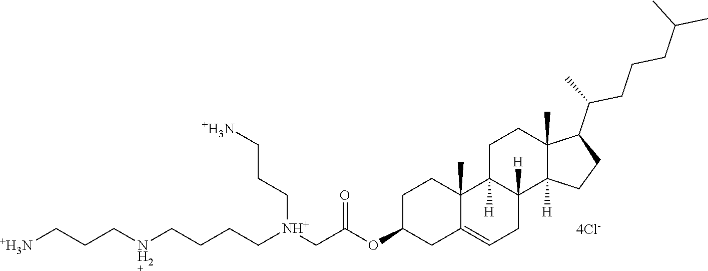

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate

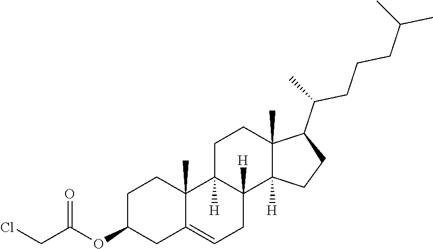

Cholesterol (2 g, 5.17 mmol), chloroacetic acid (573 mg, 5.69 mmol), DMAP (63 mg, 0.52 mmol), and DCC (1.17 g, 5.69 mmol) were dissolved in 10 mL DCM. The solution was stirred at room temperature for 17 hours. The mixture was filtered, and the filtrate was washed with ethyl acetate. The filtered solution was concentrated and dissolved in ethyl acetate. The organic layer was washed once with water and brine, dried (MgSO₄), filtered, and concentrated. The residue was purified by silica gel chromatography (0-40% ethyl acetate in hexanes) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate (0.71 g, 1.53 mmol, 30%) as a white solid. UPLC/ELSD: RT=3.43 min. ¹H NMR (300 MHz, CDCl₃) δ: ppm 5.40 (d, 1H, J=5 Hz); 4.48 (septet, 1H, J=4 Hz); 4.03 (s, 2H); 2.36 (d, 2H, J=8 Hz); 2.06-1.77 (m, 5H); 1.64-1.05 (m, 21H); 1.02 (s, 3H); 0.91 (d, 3H, J=6 Hz); 0.88 (s, 3H); 0.86 (s, 3H); 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate

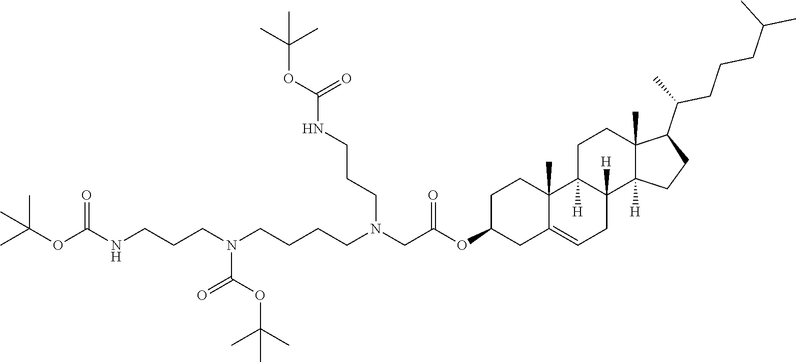

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloroacetate (350 mg, 0.75 mmol) and NaI (113 mg, 0.75 mmol) in 7.5 mL acetonitrile at room temperature was added a solution of tert-butyl N-{3-[(tert-butoxycarbonyl)amino]propyl}-N-[4-({3-[(tert-butoxycarbonyl)amino]propyl}amino)butyl]carbamate (378 mg, 0.75 mmol) and N,N-diisopropylethylamine (0.2 mL, 1.13 mmol) in 7.5 mL acetonitrile. The solution was stirred at 60° C. for 18 hours. The mixture was diluted with ethyl acetate, washed once with water and brine, dried over MgSO₄, filtered, and concentrated. The residue was purified by silica gel chromatography (0-100% (mixture of 1% NH₄OH, 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (590 mg, 0.63 mmol, 84%) as a colorless oil. UPLC/ELSD: RT=2.94 min. MS (ES): m/z (MH⁺) 930.0 for C₅₄H₉₇N₄O₈. ¹H NMR (300 MHz, CDCl₃) δ: ppm 5.36 (d, 1H, J=5 Hz); 5.27 (br s, 1H); 4.80 (br s, 1H); 4.74-4.57 (m, 1H); 3.40 (br s, 1H); 3.31-2.99 (m, 7H); 2.75 (br s, 3H); 2.31 (d, 2H, J=8 Hz); 2.06-1.03 (m, 64H); 1.00 (s, 3H); 0.91 (d, 3H, J=6 Hz); 0.87 (s, 3H); 0.85 (s, 3H); 0.67 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

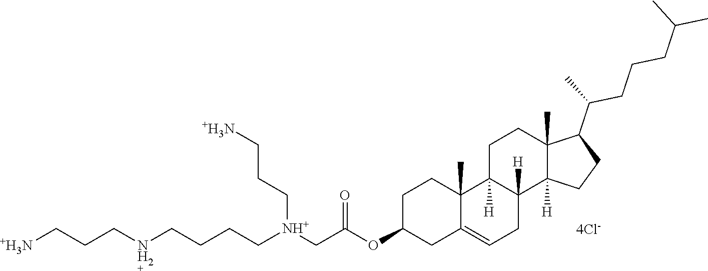

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (590 mg, 0.64 mmol) in isopropanol (15 mL) was added a 5 M HCl solution in isopropanol (15 mL, 6.4 mmol). The solution was stirred at 40° C. for 41 hours. The mixture was cooled to room temperature and diluted with acetonitrile (15 mL). Resulting solid was precipitated by centrifugation (5000 g, 5 min). The supernatant was removed, and the pellet was dried under vacuum to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate (350 mg, 0.45 mmol, 71%) as a white powder. UPLC/ELSD: RT=1.83 min. MS (ES): m/z ([M−3HCl—Cl⁻]⁺) 629.6 for C₃₉H₇₃N₄O₂. ¹H NMR (300 MHz, CD₃OD) δ: ppm 5.43 (d, 1H, J=4 Hz); 4.80-4.66 (m, 1H); 4.28 (s, 2H); 3.49-3.33 (m, 4H); 3.22-3.02 (m, 8H); 2.43 (d, 2H, J=7 Hz); 2.28-1.09 (m, 29H); 1.06 (s, 3H); 0.95 (d, 3H, J=6 Hz); 0.89 (s, 3H); 0.87 (s, 3H); 0.73 (s, 3H).

### M. Compound SA66: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride

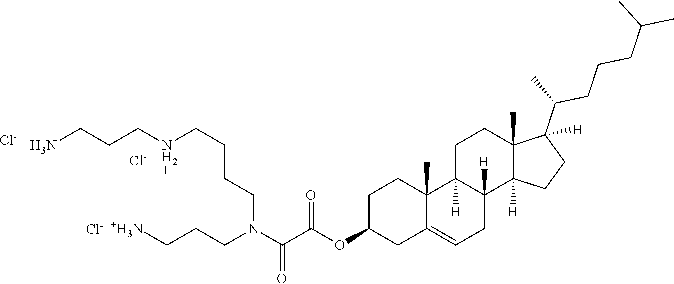

### Step 1: 2-(((3S,8S, 9S, 10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid

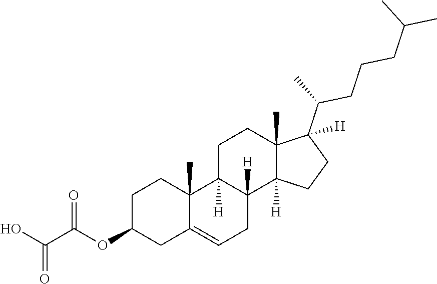

To a stirred solution of cholesterol (0.500 g, 1.29 mmol) in a mixture of Et₂O (6.5 mL) and DCM (2.0 mL) cooled to 0° C. in an ice bath was added oxalyl chloride (0.23 mL, 2.7 mmol) slowly dropwise. The reaction mixture was allowed to come to rt slowly and was monitored by TLC. At 24 h, the reaction mixture was cooled to 0° C. in an ice bath, and then water (3.0 mL) was added dropwise (CAUTION: VIGOROUS GAS EVOLUTION OCCURRED DURING ADDITION). The mixture stirred at rt for 1 h, and then the layers were separated. The aqueous layer was extracted with Et₂O (3×). The combined organics were washed with brine, dried over Na₂SO₄, and concentrated to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid (0.534 g, 1.16 mmol, 90.0%) as a white solid. UPLC/ELSD: RT=2.95 min. ¹H NMR (300 MHz, CDCl₃): δ 5.68 (br. s, 1H), 5.38-5.46 (m, 1H), 4.75-4.89 (m, 1H), 2.35-2.61 (m, 2H), 1.70-2.11 (br. m, 6H), 0.93-1.65 (br. m, 20H), 1.04 (s, 3H), 0.92 (d, 3H, J=6.5 Hz), 0.87 (d, 3H, J=6.6 Hz), 0.86 (d, 3H, J=6.5 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate

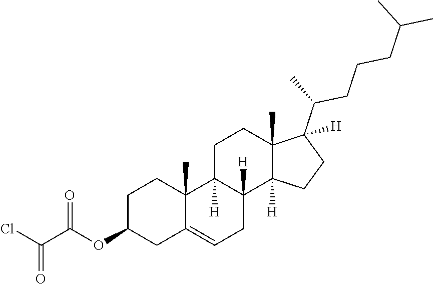

To a solution of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoacetic acid (0.100 g, 0.218 mmol) and DMF (cat.) in DCM (2 mL) was added oxalyl chloride (0.03 mL, 0.4 mmol) slowly dropwise. The reaction mixture stirred at rt and was monitored by LCMS. At 40 min, the reaction mixture was concentrated, and then reconcentrated from PhMe to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate as a yellow solid. Material was carried forward without further purification assuming quantitative yield.

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate

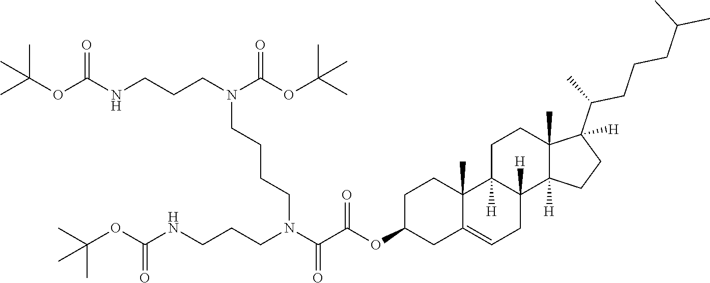

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-chloro-2-oxoacetate (0.104 g, 0.218 mmol) and triethylamine (0.10 mL, 0.71 mmol) in toluene (2.0 mL) cooled to 0° C. in an ice bath was added tert-butyl N-{3-[(tert-butoxycarbonyl)amino]propyl}-N-[4-({3-[(tert-butoxycarbonyl)amino]propyl}amino)butyl]carbamate (0.150 g, 0.298 mmol) in toluene (0.75 mL) dropwise. The reaction mixture stirred at rt and was monitored by LCMS. At 30 min, the reaction mixture stirred at 50° C. At 17 h, the reaction mixture was cooled to rt and then concentrated. The residue was taken up in DCM and washed with 5% aq. NaHCO₃ solution. The organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate (0.138 g, 0.146 mmol, 67.1%) as a yellow oil. UPLC/ELSD: RT=3.33 min. MS (ES): m/z=844.4 [(M+H)—(CH₃)₂C═CH₂—CO₂]⁺ for C₅₄H₉₄N₄O₉; ¹H NMR (300 MHz, CDCl₃): δ 5.38-5.45 (m, 1H), 5.21 (br. s, 1H), 4.65-4.87 (m, 2H), 3.32-3.47 (m, 2H), 3.02-3.31 (br. m, 10H), 2.35-2.53 (m, 2H), 0.94-2.08 (br. m, 34H), 1.46 (s, 9H), 1.44 (s, 18H), 1.02 (s, 3H), 0.92 (d, 3H, J=6.4 Hz), 0.87 (d, 3H, J=6.6 Hz), 0.86 (d, 3H, J=6.5 Hz), 0.68 (s, 3H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride

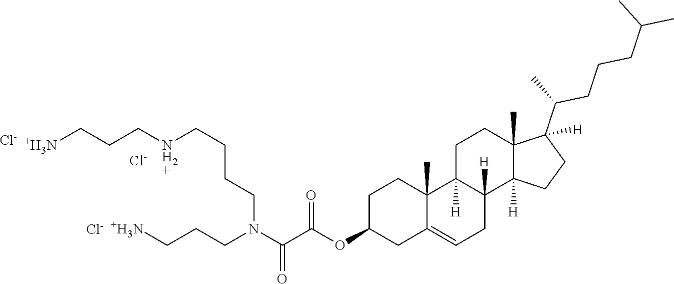

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazahexadecan-16-oate (0.132 g, 0.140 mmol) in iPrOH (1.3 mL) was added 5-6 N HCl in iPrOH (0.28 mL). The reaction mixture stirred at 40° C. and was monitored by LCMS. At 18 h, the reaction mixture was cooled to rt. ACN (3 mL) was added to the reaction mixture, and the suspension stirred at rt for 1 h. After this time, solids were collected by vacuum filtration to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((3-aminopropyl)(4-((3-aminopropyl)amino)butyl)amino)-2-oxoacetate trihydrochloride (0.085 g, 0.10 mmol, 73.2%) as a white solid. UPLC/ELSD: RT=1.70 min. MS (ES): m/z=643.8 [M+H]⁺ for C₃₉H₇₀N₄O₃; ¹H NMR (300 MHz, CD₃OD): δ 5.42-5.51 (m, 1H), 4.72-4.85 (m, 1H), 3.34-3.61 (br. m, 4H), 3.04-3.19 (br. m, 6H), 2.92-3.01 (m, 2H), 2.37-2.54 (m, 2H), 0.98-2.19 (br. m, 34H), 1.08 (s, 3H), 0.96 (d, 3H, J=6.4 Hz), 0.89 (d, 3H, J=6.6 Hz), 0.89 (d, 3H, J=6.6 Hz), 0.74 (s, 3H).

### N. Compound SA67: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-(dimethylamino)propoxy)butyl)(3-(dimethylamino)propyl)carbamate

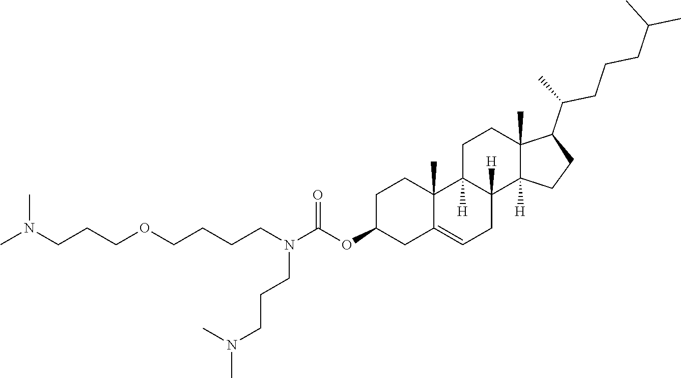

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-aminopropoxy)butyl)(3-aminopropyl)carbamate (100 mg, 0.15 mmol) and sodium acetate trihydrate (197.5 mg, 1.45 mmol) in 5.8 mL methanol at room temperature was added formaldehyde (0.11 mL, 37 wt % in water, 1.45 mmol) and sodium cyanoborohydride (91.2 mg, 1.45 mmol). The solution was stirred at room temperature for 6 hours, after which no starting aminosterol remained by LCMS. The mixture was diluted with 2 M aqueous NaOH solution and extracted three times with DCM. The organics were combined, washed once with brine, dried (MgSO₄), filtered, and concentrated. The residue was purified by silica gel chromatography (0-20% (mixture of 1% NH₄OH and 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-(3-(dimethylamino)propoxy)butyl)(3-(dimethylamino)propyl)carbamate (35.6 mg, 0.051 mmol, 35%) as a colorless oil. UPLC/ELSD: RT=2.01 min. MS (ES): m/z (MH⁺) 673.0 for C₄₂H₇₈N₃O₃. ¹H NMR (300 MHz, CDCl₃) δ: ppm 5.36 (d, 1H, J=5 Hz); 4.49 (septet, 1H, J=5 Hz); 3.48-3.37 (m, 4H); 3.23 (s, 4H); 2.48-2.30 (m, 12H); 2.28 (s, 6H); 2.24 (s, 6H); 2.04-1.66 (m, 10H); 1.64-1.04 (m, 15H); 1.01 (s, 6H); 0.91 (d, 3H, J=6 Hz); 0.87 (d, 3H, J=1 Hz); 0.85 (d, 3H, J=1 Hz); 0.67 (s, 3H).

### O. Compound SA68: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(dimethylamino)propyl)(4-((3-(dimethylamino)propyl)(methyl)amino)butyl)carbamate

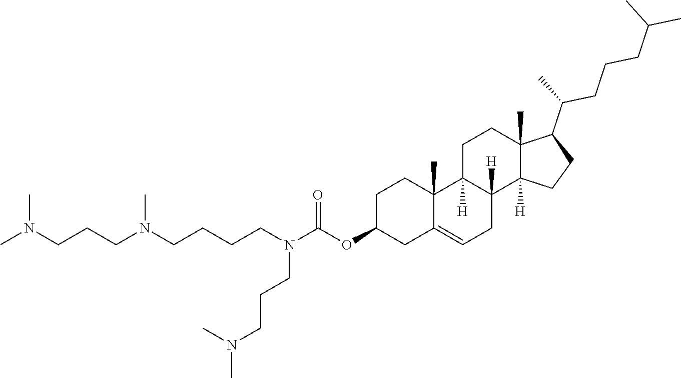

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate trihydrochloride (144 mg, 0.2 mmol) and sodium acetate trihydrate (162.3 mg, 1.19 mmol) in 2 mL methanol at room temperature was added formaldehyde (0.094 mL, 37 wt % in water, 1.19 mmol) and sodium cyanoborohydride (75 mg, 1.19 mmol). The solution was stirred at room temperature for 17 hours, after which no starting aminosterol remained by LCMS. The mixture was diluted with 2 M aqueous NaOH solution and extracted three times with DCM. The organics were combined, washed once with brine, dried (MgSO₄), filtered and concentrated. The residue was purified by silica gel chromatography (0-20% (mixture of 2% concentrated aq. NH₄OH and 20% MeOH in DCM) in DCM) to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-(dimethylamino)propyl)(4-((3-(dimethylamino)propyl)(methyl)amino)butyl)carbamate (31.7 mg, 0.044 mmol, 22%) as a colorless oil. UPLC/ELSD: RT=1.71 min. MS (ES): m/z (MH⁺) 685.6 for C₄₃H₈₁N₄O₂. ¹H NMR (300 MHz, CDCl₃) δ: ppm 5.36 (d, 1H, J=5 Hz); 4.49 (septet, 1H, J=5 Hz); 3.47 (s, 4H); 3.22 (s, 4H); 2.38-2.24 (m, 12H); 2.22 (s, 6H); 2.21 (s, 6H); 2.20 (s, 3H); 2.05-1.95 (m, 2H); 1.72-1.06 (m, 23H); 1.01 (s, 6H); 0.91 (d, 3H, J=6 Hz); 0.87 (d, 3H, J=1 Hz); 0.85 (d, 3H, J=1 Hz); 0.67 (s, 3H).

### P. Compound SA69: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

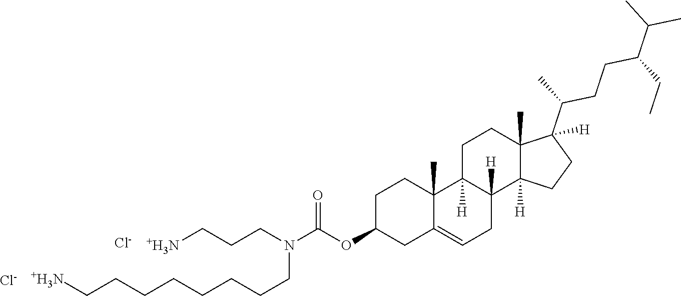

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate

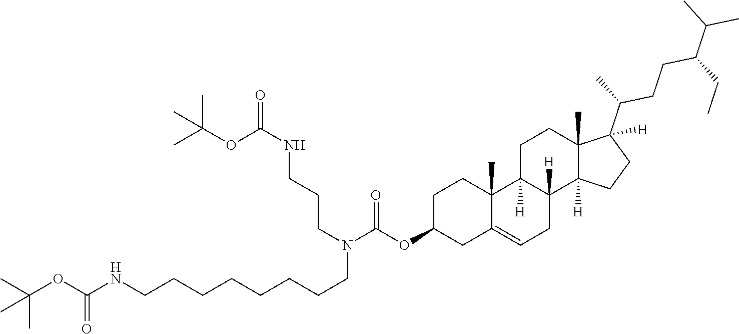

β-Sitosterol 4-nitrophenyl carbonate (0.300 g, 0.517 mmol), tert-butyl N-[3-({8-[(tert-butoxycarbonyl)amino]octyl}amino)propyl]carbamate (0.260 g, 0.647 mmol), and triethylamine (0.22 mL, 1.6 mmol) were combined in PhMe (4.5 mL). The reaction mixture stirred at 90° C. and was monitored by LCMS. At 18.25 h, the reaction mixture was cooled to rt and concentrated. The residue was taken up in DCM (20 mL) and washed with water (3×). The organic layer was passed through a hydrophobic frit, dried over Na₂SO₄, and concentrated. The crude material was purified via silica gel chromatography (20-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.327 g, 0.388 mmol, 75.0%) as a white foam. UPLC/ELSD: RT=3.74 min. MS (ES): m/z=842.9 [M+H]⁺ for C₅₁H₉₁N₃O₆; ¹H NMR (300 MHz, CDCl₃): δ 5.15-5.47 (m, 2H), 4.40-4.86 (m, 2H), 2.98-3.41 (br. m, 8H). 2.20-2.45 (m, 2H), 1.76-2.12 (br. m, 5H), 0.89-1.75 (br. m, 54H), 1.02 (s, 3H), 0.92 (d, 3H, J=6.4 Hz), 0.77-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride

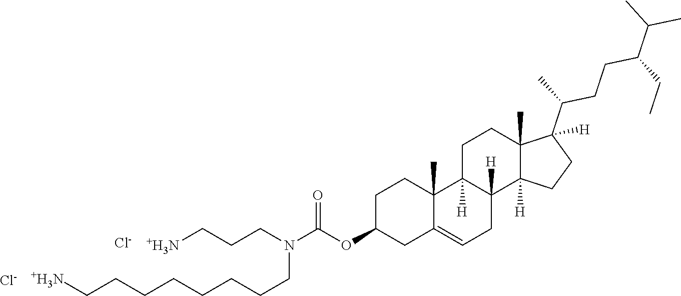

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-((tert-butoxycarbonyl)amino)octyl)(3-((tert-butoxycarbonyl)amino)propyl)carbamate (0.315 g, 0.374 mmol) in iPrOH (4.0 mL) was added 5-6 N HCl in iPrOH (0.53 mL). The reaction mixture stirred at 40° C. and was monitored by LCMS. At 18 h, the reaction mixture was cooled to rt and ACN (12 mL) was added. The solids were collected via vacuum filtration and rinsed with 3:1 ACN/iPrOH to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (8-aminooctyl)(3-aminopropyl)carbamate dihydrochloride (0.236 g, 0.309 mmol, 82.5%) as a white solid. UPLC/ELSD: RT=3.54 min. MS (ES): m/z=342.6 [M+2Na]²⁺ for C₄₁H₇₇Cl₂N₃O₂; ¹H NMR (300 MHz, CDCl₃): δ 8.33 (br. s, 3H), 8.22 (br. s, 3H), 5.31-5.42 (m, 1H), 4.38-4.53 (m, 1H), 2.92-3.53 (br. m, 8H), 2.20-2.42 (m, 2H), 1.72-2.17 (br. m, 10H), 0.94-1.71 (br. m, 31H), 1.02 (s, 3H), 0.92 (d, 3H, J=6.3 Hz), 0.77-0.89 (m, 9H), 0.68 (s, 3H).

### Q. Compound SA70

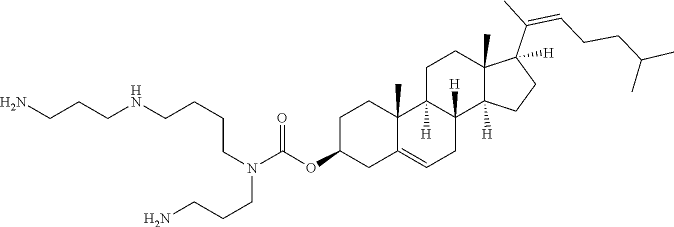

SA70 ((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopropyl)(4-((3-aminopropyl)amino)butyl)carbamate analog olefin elimination by-product from hydroxycholesterol)

### R. Compound SA71: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride

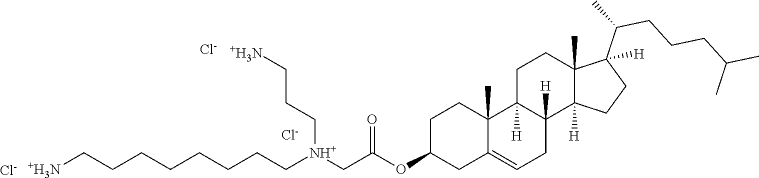

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate

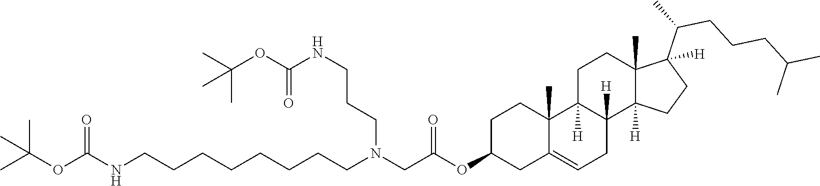

Cholesteryl chloroacetate (0.227 g, 0.490 mmol), tert-butyl N-[3-({8-[(tert-butoxycarbonyl)amino]octyl}amino)propyl]carbamate (0.236 g, 0.589 mmol), potassium carbonate (0.136 g, 0.980 mmol), and potassium iodide (0.008 g, 0.05 mmol) were combined in THE (3.5 mL). The reaction mixture stirred at 65° C. and was monitored by LCMS. At 4 h, the reaction mixture stirred at 60° C. At 93 h, the reaction mixture was cooled to rt. The reaction mixture was concentrated and then taken up in DCM. The organics was washed with water, passed through a hydrophobic frit, dried over Na₂SO₄, and concentrated. The crude material was purified via silica gel chromatography (20-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate (0.318 g, 0.384 mmol, 78.3%) as a clear oil. UPLC/ELSD: RT=3.62 min. MS (ES): m/z=829.0 [M+H]⁺ for C₅₀H₈₉N₃O₆; ¹H NMR (300 MHz, CDCl₃): δ 5.45 (br. s, 1H), 5.35-5.41 (m, 1H), 4.58-4.72 (m, 1H), 4.50 (br. s, 1H), 3.25 (s, 2H), 3.20 (dt, 2H, J=5.7, 6.0 Hz), 3.09 (dt, 2H, J=6.4, 5.8 Hz), 2.59 (t, 2H, J=6.4 Hz), 2.50 (t, 2H, J=7.5 Hz), 2.28-2.36 (m, 2H), 1.75-2.08 (br. m, 5H), 0.94-1.70 (br. m, 53H), 1.02 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride

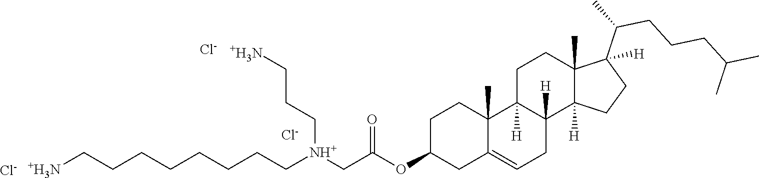

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(3-((tert-butoxycarbonyl)amino)propyl)glycinate (0.310 g, 0.374 mmol) in iPrOH (4.0 mL) was added 5-6 N HCl in iPrOH (0.53 mL). The reaction mixture stirred at 40° C. and was monitored by LCMS. At 21.75 h, the reaction mixture was cooled to rt, and ACN (12 mL) was added. The solids were collected via vacuum filtration and rinsed with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(3-aminopropyl)glycinate trihydrochloride (0.208 g, 0.248 mmol, 66.4%) as a white solid. UPLC/ELSD: RT=1.83 min. MS (ES): m/z=335.4 [M+2Na]²⁺ for C₄₀H₇₃N₃O₂; ¹H NMR (300 MHz, CDCl₃): δ 10.72 (br. s, 1H), 8.41 (br. s, 3H), 8.27 (br. s, 3H), 5.38-5.48 (m, 1H), 4.59-4.82 (m, 1H), 2.91-4.42 (br. m, 10H), 2.22-2.72 (br. m, 4H), 1.72-2.18 (br. m, 10H), 0.93-1.70 (br. m, 28H), 1.01 (s, 3H), 0.91 (d, 3H, J=5.5 Hz), 0.86 (d, 6H, J=6.5 Hz), 0.67 (s, 3H).

### S. Compound SA72: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride

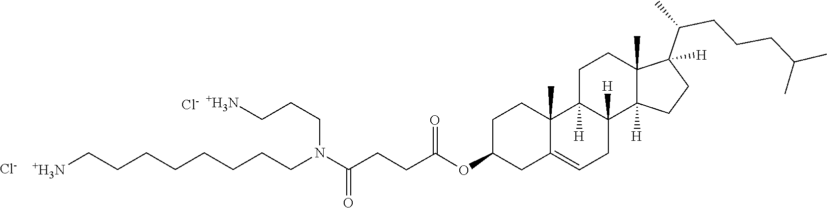

### Step 1: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate

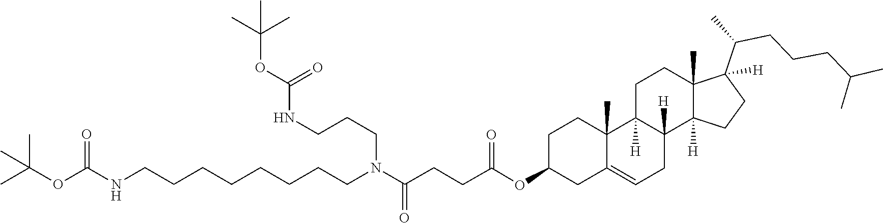

To a stirred solution of (−)-cholesterol NHS succinate (0.300 g, 0.514 mmol) in THE (3.0 mL) was added tert-butyl N-[3-({8-[(tert-butoxycarbonyl)amino]octyl}amino)propyl]carbamate (0.258 g, 0.642 mmol) in THE (1.0 mL). The reaction mixture stirred at rt and was monitored by LCMS. At 19 h, the reaction mixture was stirred at 50° C. At 23 h, the reaction mixture was cooled to rt and then concentrated. The residue was taken up in DCM and washed with water. The organics were passed through a hydrophobic frit, dried over Na₂SO₄, and concentrated. The crude material was purified via silica gel chromatography (20-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate (0.315 g, 0.362 mmol, 70.4%) as a white foam. UPLC/ELSD: RT=3.96 min. MS (ES): m/z=871.0 [M+H]⁺ for C₅₂H₉₁N₃O₇; ¹H NMR (300 MHz, CDCl₃): δ 5.27-5.46 (m, 2H), 4.39-4.76 (m, 2H), 2.95-3.48 (br. m, 8H), 2.53-2.72 (m, 4H), 2.24-2.39 (m, 2H), 1.75-2.06 (br. m, 5H), 0.93-1.70 (br. m, 53H), 1.01 (s, 3H), 0.91 (d, 3H, J=6.4 Hz), 0.86 (d, 3H, J=6.5 Hz), 0.86 (d, 3H, J=6.6 Hz), 0.67 (s, 3H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride

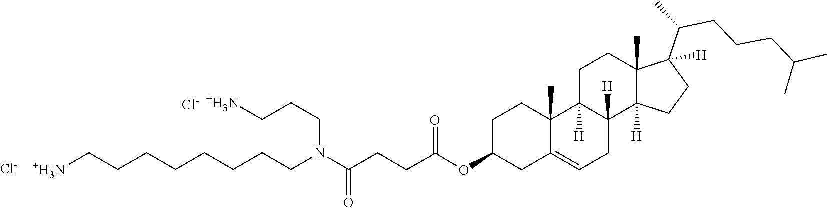

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-((tert-butoxycarbonyl)amino)octyl)(3-((tert-butoxycarbonyl)amino)propyl)amino)-4-oxobutanoate (0.307 g, 0.347 mmol) in iPrOH (4 mL) was added 5-6 N HCl in iPrOH (0.49 mL). The reaction mixture stirred at 40° C. and was monitored by LCMS. At 21.75 h, the reaction mixture was cooled to rt and then ACN (16 mL) was added. The solids were collected via vacuum filtration and rinsed with 4:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((8-aminooctyl)(3-aminopropyl)amino)-4-oxobutanoate dihydrochloride (0.169 g, 0.214 mmol, 61.8%) as a white solid. UPLC/ELSD: RT=2.15 min. MS (ES): m/z=336.0 [M+2H]²⁺ for C₄₂H₇₅N₃O₃; ¹H NMR (300 MHz, CDCl₃): δ 8.01-8.61 (m, 6H), 5.31-5.42 (m, 1H), 4.51-4.69 (m, 1H), 2.92-3.68 (br. m, 8H), 2.62 (s, 4H), 2.21-2.39 (m, 2H), 1.71-2.20 (br. m, 10H), 0.94-1.70 (br. m, 30H), 1.01 (s, 3H), 0.91 (d, 3H, J=6.4 Hz), 0.86 (d, 6H, J=6.5 Hz), 0.67 (s, 3H).

### T. Compound SA73: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride

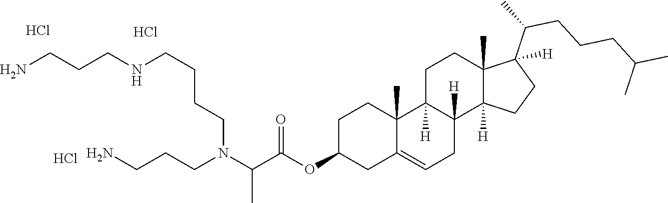

### Step 1: 9-(tert-Butoxycarbonyl)-14-(3-((tert-butoxycarbonyl)amino)propyl)-2,2,15-trimethyl-4-oxo-3-oxa-5,9,14-triazahexadecan-16-oic acid

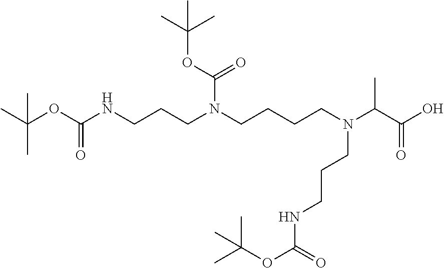 [SMILES: CC(C(=O)O)N(CCCCN(CCCNC(=O)OC(C)(C)C)C(=O)OC(C)(C)C)CCCNC(=O)OC(C)(C)C]

To a solution of tert-butyl (3-((tert-butoxycarbonyl)amino)propyl)(4-((3-((tert-butoxycarbonyl)amino)propyl)amino)butyl)carbamate (0.83 g, 1.65 mmol) and potassium hydroxide (0.37 g, 6.60 mmol) in methanol (10 mL) stirring under nitrogen was added 2-bromopropionic acid (0.30 mL, 3.30 mmol) dropwise at room temperature. The resulting solution was heated to 60° C. and allowed to proceed overnight. The following day, the solution was concentrated to an oil. The oil was taken up in dichloromethane and purified on silica with a 0-60% ethyl acetate gradient in hexanes to give 9-(tert-butoxycarbonyl)-14-(3-((tert-butoxycarbonyl)amino)propyl)-2,2,15-trimethyl-4-oxo-3-oxa-5,9,14-triazahexadecan-16-oic acid as an oil (0.13 g, 0.22 mmol, 13.2%). UPLC/ELSD: RT: 2.73 min. MS (ES): m/z (MH⁺) 575.8 for C₂₈H₅₄N₄O₈. ¹H NMR (300 MHz, CDCl₃) δ: ppm 7.31 (br. s, 1H), 5.72 (br. s, 1H), 3.17 (br. m, 13H), 1.90 (br. m, 2H), 1.64 (br. m, 7H), 1.40 (s, 26H).

### Step 2: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate

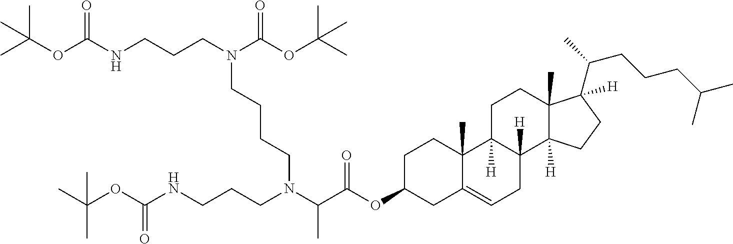

To a solution of cholesterol (0.10 g, 0.26 mmol) and 9-(tert-butoxycarbonyl)-14-(3-((tert-butoxycarbonyl)amino)propyl)-2,2,15-trimethyl-4-oxo-3-oxa-5,9,14-triazahexadecan-16-oic acid (0.13 g, 0.22 mmol) in dichloromethane (10 mL) stirring under nitrogen was added dimethylaminopyridine (0.01 g, 0.04 mmol) and 1-ethyl-3-(3-dimethylaminopropyl)carbodiimide hydrochloride (0.06 g, 0.33 mmol). The resulting solution was cooled to 0° C. and diisopropylethylamine (0.12 mL, 0.65 mmol) was added dropwise. The mixture was allowed to gradually warm to room temperature and proceed overnight. Then, the solution was diluted with dichloromethane, washed with water (1×10 mL), saturated aqueous sodium bicarbonate (1×10 mL) and brine (1×10 mL), dried over sodium sulfate, filtered, and concentrated to an oil. The oil was taken up in DCM and purified on silica with a 0-25% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate as a colorless oil (0.05 g, 0.05 mmol, 21.9%). UPLC/ELSD: RT: 2.83 min. MS (ES): m/z (MH⁺) 944.4 for C₅₅H₉₈N₄O₈. The compound was not analyzed by H-NMR so as not to lose precious material needed for the following reaction.

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride

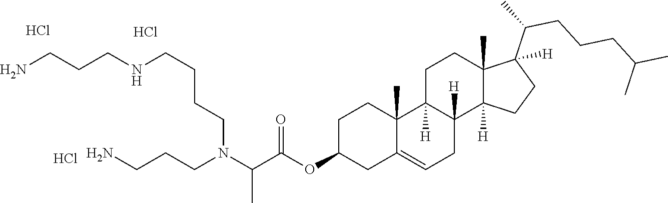

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate (0.05 g, 0.05 mmol) in 2-propanol (5 mL) stirring under nitrogen was added hydrochloric acid (5.5M in 2-propanol, 0.10 mL, 0.48 mmol) dropwise. The mixture was heated to 45° C. and allowed to stir overnight. Then, the solution was cooled to room temperature, and acetonitrile (3 mL) was added to the mixture. It was then sonicated to remove precipitated solid off the side of the flask. After stirring for 30 minutes after sonication, the solid was filtered out by vacuum filtration, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate trihydrochloride as a white solid (0.03 g, 0.03 mmol, 62.8%). UPLC/ELSD: RT: 1.51 min. MS (ES): m/z (MH⁺) 644.1 for C₄₀H₇₇Cl₃N₄O₂. ¹H NMR (300 MHz, CD₃OD) δ: ppm 5.54 (br. s, 1H), 4.50 (br. m, 1H), 3.33 (br. d, 8H), 3.12 (br. m, 9H), 2.45 (br. m, 2H), 2.00 (br. m, 15H), 1.55 (br. m, 17H), 1.19 (br. m, 14H), 0.96 (d, 4H, J=6 Hz), 0.90 (d, 7H, J=6 Hz), 0.74 (s, 3H).

### U. Compound SA74: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

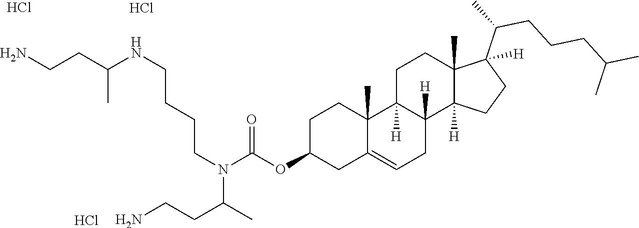

### Step 1: tert-butyl (3-((4-nitrophenyl)sulfonamido)butyl)carbamate

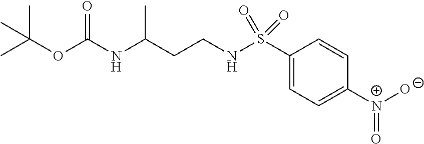 [SMILES: CC(CCNS(=O)(=O)c1ccc([N+](=O)[O-])cc1)NC(=O)OC(C)(C)C]

To a solution of tert-butyl (3-aminobutyl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1 M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (3-((4-nitrophenyl)sulfonamido)butyl)carbamate as a white solid (1.95 g, 5.22 mmol, 98.3%). UPLC/ELSD: RT=0.54 min. MS (ES): m/z (MH⁺) 374.4 for C₁₅H₂₃N₃O₆S. ¹H NMR (300 MHz, CDCl₃) δ: ppm 8.07 (m, 1H), 7.78 (m, 1H), 7.68 (m, 1H), 5.23 (m, 1H), 4.81 (br. s, 1H), 3.52 (m, 1H), 3.19 (m, 1H), 3.05 (m, 1H), 1.63 (m, 2H), 1.37 (s, 9H), 0.98 (d, 3H, J=6 Hz).

### Step 2: di-tert-butyl ((butane-1,4-diylbis(azanediyl))bis(butane-3,1-diyl))dicarbamate

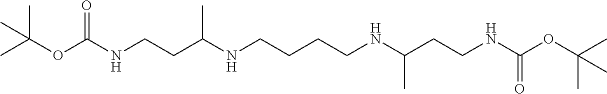 [SMILES: CC(CCNC(=O)OC(C)(C)C)NCCCCNC(C)CCNC(=O)OC(C)(C)C]

To a solution of tert-butyl (3-((4-nitrophenyl)sulfonamido)butyl)carbamate (1.95 g, 5.22 mmol) in dry DMF (20 mL) set stirring under nitrogen was added potassium carbonate (2.10 g, 15.17 mmol) and 1,4-diiodobutane (0.33 mL, 2.49 mmol). The solution was heated to 40° C. and allowed to proceed overnight. The following morning, benzyl bromide (0.25 mL, 2.06 mmol) was added, and the reaction was allowed to proceed at room temperature for 24 h. Then, thiophenol (0.98 mL, 9.57 mmol), potassium carbonate (1.03 g, 7.46 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(butane-3,1-diyl))dicarbamate as a colorless oil (0.76 g, 1.77 mmol, 71.0%). UPLC/ELSD: RT=0.42 min. MS (ES): m/z (MH⁺) 431.6 for C₂₂H₄₆N₄O₄. ¹H NMR (300 MHz, CDCl₃) δ: ppm 5.47 (m, 2H), 3.24 (br. m, 4H), 2.74 (br. m, 4H), 2.55 (m, 2H), 1.53 (m, 10H), 1.44 (s, 18H), 1.09 (d, 6H, J=6 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate

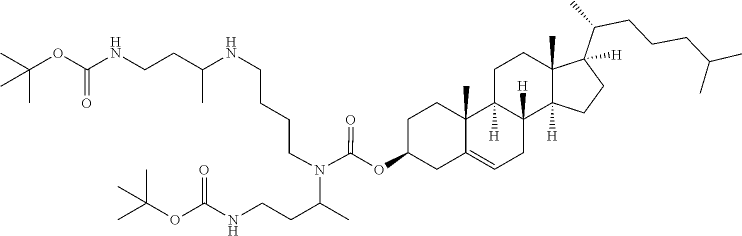

To a solution of di-tert-butyl ((butane-1,4-diylbis(azanediyl))bis(butane-3,1-diyl))dicarbamate (0.49 g, 1.15 mmol) in dry toluene (10 mL) set stirring under nitrogen was added triethylamine (0.48 mL, 3.43 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.63 g, 1.15 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to give an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate as a colorless oil (0.78 g, 0.92 mmol, 80.6%). UPLC/ELSD: RT=2.62 min. MS (ES): m/z (MH⁺) 844.3 for C₅₀H₉₀N₄O₆. ¹H NMR (300 MHz, CDCl₃) δ: ppm 5.28 (m, 1H), 3.14 (br. m, 5H), 2.59 (m, 4H), 2.25 (m, 3H), 1.90 (br. m, 7H), 1.46 (br. m, 22H), 1.34 (s, 23H), 1.09 (br. m, 28H), 0.83 (d, 5H, J=6 Hz), 0.79 (d, 7H, J=6 Hz), 0.59 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

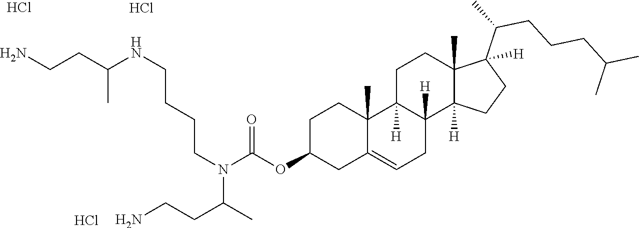

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate (0.78 g, 0.92 mmol) in isopropanol (10 mL) stirring under nitrogen was added hydrochloric acid (5 N in isopropanol, 1.85 mL, 9.23 mmol) dropwise. The solution was heated to 40° C. and allowed to proceed overnight. The following morning, dry acetonitrile (6 mL) was added to the mixture, which was sonicated and allowed to stir for an additional hour. The white solid was then filtered out of the solution, washed repeatedly with acetonitrile, and dried in vacuo to give SA74 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride as a white solid (0.57 g, 0.73 mmol, 78.8%). UPLC/ELSD: RT=1.67 min. MS (ES): m/z (MH⁺) 753.4 for C₄₀H₇₇Cl₃N₄O₂. ¹H NMR (300 MHz, CDCl₃) δ: ppm 5.42 (m, 1H), 4.49 (br. m, 1H), 4.12 (br. m, 1H), 3.45 (br. m, 1H), 3.33 (s, 2H), 3.24 (br. m, 2H), 3.13 (br. m, 4H), 2.90 (br. m, 2H), 2.41 (d, 2H, J=3 Hz), 2.32 (br. m, 1H), 1.93 (br. m, 19H), 1.43 (d, 6H, J=6 Hz), 1.31 (d, 5H, J=6 Hz), 1.08 (br. m, 12H), 0.97 (d, 4H, J=6 Hz), 0.90 (d, 6H, J=6 Hz), 0.75 (s, 3H).

### V. Compound SA75: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride

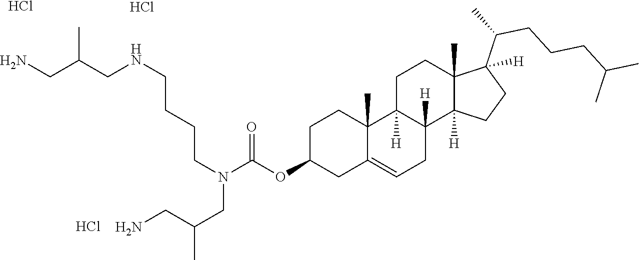

### Step 1: tert-butyl (2-methyl-3-((4-nitrophenyl)sulfonamido)propyl)carbamate

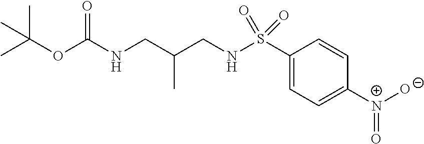 [SMILES: CC(CNC(=O)OC(C)(C)C)CNS(=O)(=O)c1ccc([N+](=O)[O-])cc1]

To a solution of tert-butyl (3-amino-2-methylpropyl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-methyl-3-((4-nitrophenyl)sulfonamido)propyl)carbamate as a white solid (2.20 g, 5.90 mmol, quantitative). UPLC/ELSD: RT=0.58 min. MS (ES): m/z (MH⁺) 374.4 for C₁₅H₂₃N₃O₆S. ¹H NMR (300 MHz, CDCl₃) δ: ppm 8.12 (m, 1H), 7.84 (m, 1H), 7.74 (m, 1H), 6.20 (br. s, 1H), 4.82 (br. s, 1H), 3.19 (m, 1H), 3.04 (m, 3H), 1.84 (m, 1H), 1.41 (s, 9H), 0.91 (d, 3H, J=6 Hz).

### Step 2: di-tert-butyl ((butane-1,4-diylbis(azanediyl))bis(2-methylpropane-3,1-diyl))dicarbamate

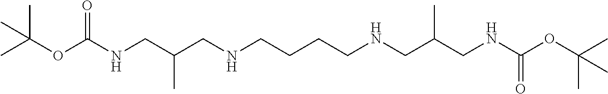 [SMILES: CC(CNCCCCNCC(C)CNC(=O)OC(C)(C)C)CNC(=O)OC(C)(C)C]

To a solution of tert-butyl (2-methyl-3-((4-nitrophenyl)sulfonamido)propyl)carbamate (2.20 g, 5.90 mmol) in dry DMF (20 mL) stirring under nitrogen was added potassium carbonate (2.37 g, 17.14 mmol) and 1,4-diiodobutane (0.37 mL, 2.81 mmol). The solution was heated to 40° C. and allowed to proceed overnight. The following morning, benzyl bromide (0.28 mL, 2.33 mmol) was added, and the reaction was allowed to proceed at room temperature for 24 h. Then, thiophenol (1.11 mL, 10.82 mmol), potassium carbonate (1.17 g, 8.43 mmol), and an additional 5 mL of dry DMF were added, and the reaction was allowed to proceed overnight again. The following morning, salts were removed from the supernatant via multiple rounds of centrifugation and rinsing with DMF. The combined supernatants were concentrated in vacuo to an oil which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-methylpropane-3,1-diyl))dicarbamate as a colorless oil (0.85 g, 1.97 mmol, 70.0%). UPLC/ELSD: RT=0.43 min. MS (ES): m/z (MH⁺) 431.6 for C₂₂H₄₆N₄O₄. ¹H NMR (300 MHz, CDCl₃) δ: ppm 5.76 (m, 1H), 2.93 (m, 2H), 2.73 (m, 2H), 2.28 (m, 8H), 1.56 (m, 4H), 1.28 (s, 4H), 1.18 (s, 17H), 0.66 (d, 6H, J=6 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate

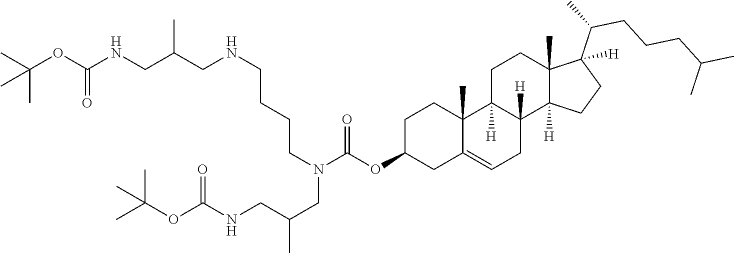

To a solution of di-tert-butyl ((butane-1,4-diylbis(azanediyl))bis(2-methylpropane-3,1-diyl))dicarbamate (1.06 g, 2.45 mmol) in dry toluene (20 mL) stirring under nitrogen was added triethylamine (0.86 mL, 6.13 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.13 g, 2.04 mmol) was added and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate as a colorless oil (1.08 g, 1.28 mmol, 62.8%). UPLC/ELSD: RT=2.52 min. MS (ES): m/z (MH⁺) 844.3 for C₅₀H₉₀N₄O₆. ¹H NMR (300 MHz, CDCl₃) δ: ppm 5.29 (m, 1H), 4.42 (br. m, 1H), 3.07 (br. m, 5H), 2.87 (m, 3H), 2.51 (m, 4H), 2.25 (br. m, 2H), 1.79 (br. m, 7H), 1.46 (m, 8H), 1.34 (s, 18H), 1.05 (br. m, 10H), 0.94 (s, 5H), 0.82 (m, 14H), 0.59 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride

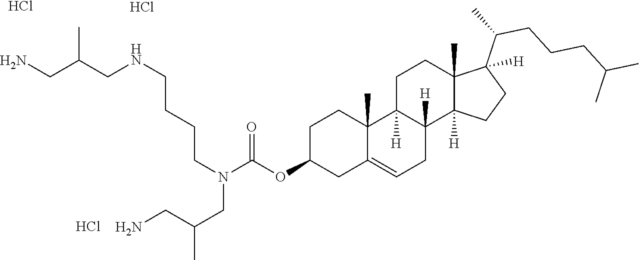

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-methylpropyl)(4-((3-((tert-butoxycarbonyl)amino)-2-methylpropyl)amino)butyl)carbamate (1.08 g, 1.28 mmol) in isopropanol (10 mL) set stirring under nitrogen was added hydrochloric acid (5 N in isopropanol, 2.57 mL, 12.83 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA75 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-methylpropyl)(4-((3-amino-2-methylpropyl)amino)butyl)carbamate trihydrochloride as a white solid (0.66 g, 0.85 mmol, 66.1%). UPLC/ELSD: RT=1.59 min. MS (ES): m/z (MH⁺) 753.4 for C₄₀H₇₇Cl₃N₄O₂. ¹H NMR (300 MHz, CDCl₃) δ: ppm 5.42 (m, 1H), 4.46 (br. m, 1H), 3.33 (br. m, 4H), 3.12 (br. m, 5H), 2.95 (m, 4H), 2.40 (d, 3H, J=9 Hz), 1.75 (br. m, 19H), 1.20 (br. m, 9H), 1.08 (m, 8H), 0.97 (d, 4H, J=6 Hz), 0.89 (d, 6H, J=6 Hz), 0.75 (s, 3H).

### W. Compound SA76: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

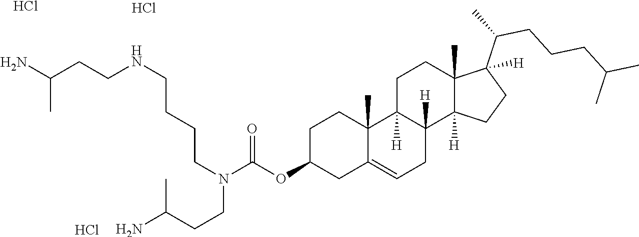

### Step 1: tert-butyl (4-((4-nitrophenyl)sulfonamido)butan-2-yl)carbamate

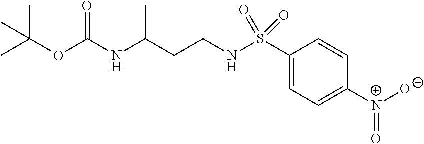 [SMILES: CC(CCNS(=O)(=O)c1ccc([N+](=O)[O-])cc1)NC(=O)OC(C)(C)C]

To a solution of tert-butyl (4-aminobutan-2-yl)carbamate (1.00 g, 5.31 mmol) in dry DCM (15 mL) stirring under nitrogen was added triethylamine (0.89 mL, 6.37 mmol). The solution was cooled to 0° C., and then a solution of 4-nitrobenzenesulfonyl chloride (1.30 g, 5.84 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (4-((4-nitrophenyl)sulfonamido)butan-2-yl)carbamate as a white solid (1.47 g, 3.94 mmol, 74.1%). UPLC/ELSD: RT=0.61 min. MS (ES): m/z (MH⁺) 374.4 for C₁₅H₂₃N₃O₆S. ¹H NMR (300 MHz, CDCl₃) δ: ppm 8.00 (m, 1H), 7.81 (m, 2H), 7.73 (m, 2H), 6.15 (br. s, 1H), 4.27 (br. s, 1H), 3.64 (br. s, 1H), 3.19 (br. s, 1H), 2.95 (br. s, 1H), 1.64 (m, 1H), 1.30 (s, 10H), 1.00 (d, 3H, J=6 Hz).

### Step 2: di-tert-butyl ((butane-1,4-diylbis(azanediyl))bis(butane-4,2-diyl))dicarbamate

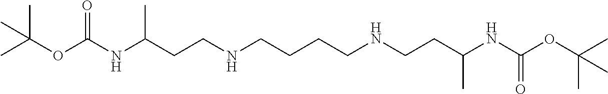 [SMILES: CC(CCNCCCCNCCC(C)NC(=O)OC(C)(C)C)NC(=O)OC(C)(C)C]

To a solution of tert-butyl (4-((4-nitrophenyl)sulfonamido)butan-2-yl)carbamate (1.47 g, 3.94 mmol) in dry DMF (20 mL) set stirring under nitrogen was added potassium carbonate (1.58 g, 11.44 mmol) and 1,4-diiodobutane (0.25 mL, 1.88 mmol). The solution was heated to 40° C. and allowed to proceed overnight. The following morning, benzyl bromide (0.19 mL, 1.56 mmol) was added, and the reaction was allowed to proceed at room temperature for 24 h. Then, thiophenol (0.74 mL, 7.22 mmol), potassium carbonate (0.78 g, 5.62 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(butane-4,2-diyl))dicarbamate as a colorless oil (0.17 g, 0.40 mmol, 21.2%). UPLC/ELSD: RT=0.45 min. MS (ES): m/z (MH⁺) 431.6 for C₂₂H₄₆N₄O₄. ¹H NMR (300 MHz, CDCl₃) δ: ppm 4.94 (m, 1H), 3.66 (m, 5H), 2.66 (m, 8H), 1.66 (m, 8H), 1.39 (s, 18H), 1.10 (d, 6H, J=9 Hz).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate

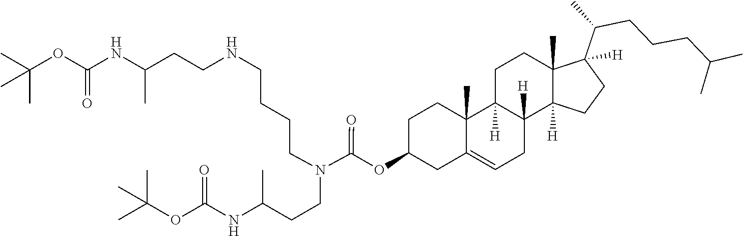

To a solution of di-tert-butyl ((butane-1,4-diylbis(azanediyl))bis(butane-4,2-diyl))dicarbamate (0.17 g, 0.40 mmol) in dry toluene (5 mL) set stirring under nitrogen was added triethylamine (0.15 mL, 1.08 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.20 g, 0.36 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate as a colorless oil (0.13 g, 0.16 mmol, 43.0%). UPLC/ELSD: RT=2.77 min. MS (ES): m/z (MH⁺) 844.3 for C₅₀H₉₀N₄O₆. ¹H NMR (300 MHz, CDCl₃) δ: ppm 5.36 (m, 1H), 4.89 (m, 1H), 4.50 (m, 2H), 3.67 (br. m, 2H), 3.22 (br. m, 4H), 2.59 (m, 4H), 2.33 (m, 2H), 1.98 (br. m, 6H), 1.54 (br. m, 15H), 1.43 (s, 22H), 1.34 (m, 5H), 1.14 (m, 14H), 1.02 (s, 7H), 0.82 (m, 14H), 0.91 (d, 4H, J=6 Hz), 0.86 (d, 6H, J=6 Hz), 0.67 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

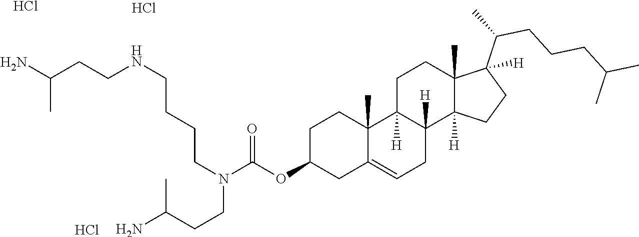

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate (0.13 g, 0.16 mmol) in isopropanol (10 mL) set stirring under nitrogen was added hydrochloric acid (5 N in isopropanol, 0.31 mL, 1.55 mmol) dropwise. The solution was heated to 4 0° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA76 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride as a white solid (0.06 g, 0.07 mmol, 47.9%). UPLC/ELSD: RT=1.49 min. MS (ES): m/z (MH⁺) 753.4 for C₄₀H₇₇Cl₃N₄O₂. ¹H NMR (300 MHz, CDCl₃) δ: ppm 5.42 (m, 1H), 4.48 (br. m, 1H), 3.47 (br. m, 2H), 3.33 (br. m, 7H), 3.17 (m, 4H), 2.40 (d, 2H, J=9 Hz), 2.05 (br. m, 8H), 1.62 (br. m, 10H), 1.39 (d, 8H, J=9 Hz), 1.16 (d, 7H, J=6 Hz), 1.08 (br. m, 5H), 0.98 (d, 3H, J=6 Hz), 0.91 (d, 5H, J=6 Hz), 0.74 (s, 3H).

### X. Compound SA77: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride

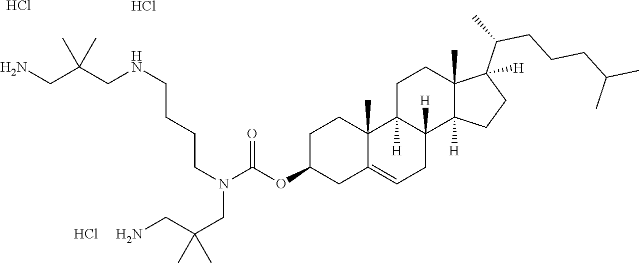

### Step 1: tert-butyl (2,2-dimethyl-3-((4-nitrophenyl)sulfonamido)propyl)carbamate

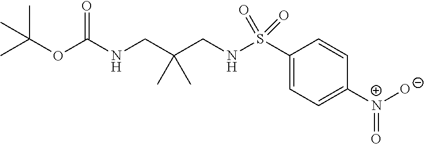 [SMILES: CC(C)(CNC(=O)OC(C)(C)C)CNS(=O)(=O)c1ccc([N+](=O)[O-])cc1]

To a solution of tert-butyl (3-amino-2,2-dimethylpropyl)carbamate (1.00 g, 4.94 mmol) in dry DCM (15 mL) set stirring under nitrogen was added triethylamine (0.83 mL, 5.93 mmol). The solution was cooled to 0° C., and then a solution of 4-nitrobenzenesulfonyl chloride (1.20 g, 5.44 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2,2-dimethyl-3-((4-nitrophenyl)sulfonamido)propyl)carbamate as a white solid (2.00 g, 5.15 mmol, 104.1%). UPLC/ELSD: RT=0.85 min. MS (ES): m/z (MH⁺) 388.4 for C₁₆H₂₅N₃O₆S. ¹H NMR (300 MHz, CDCl₃) δ: ppm 8.10 (m, 1H), 7.82 (m, 1H), 7.73 (m, 2H), 6.54 (br. s, 1H), 4.86 (br. s, 1H), 2.99 (d, 2H, J=6 Hz), 2.81 (d, 2H, J=9 Hz), 1.41 (s, 9H), 0.90 (s, 6H).

### Step 2: di-tert-butyl ((butane-1,4-diylbis(azanediyl))bis(2,2-dimethylpropane-3,1-diyl))dicarbamate

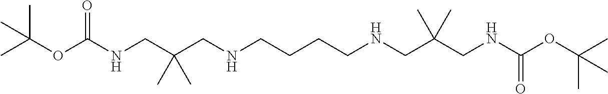 [SMILES: CC(C)(CNCCCCNCC(C)(C)CNC(=O)OC(C)(C)C)CNC(=O)OC(C)(C)C]

To a solution of tert-butyl (2,2-dimethyl-3-((4-nitrophenyl)sulfonamido)propyl)carbamate (2.00 g, 5.15 mmol) in dry DMF (20 mL) stirring under nitrogen was added potassium carbonate (2.07 g, 14.96 mmol) and 1,4-diiodobutane (0.32 mL, 2.45 mmol). The solution was heated to 40° C. and allowed to proceed overnight. The following morning, benzyl bromide (0.24 mL, 2.04 mmol) was added, and the reaction was allowed to proceed at room temperature for 24 h. Then, thiophenol (0.97 mL, 9.44 mmol), potassium carbonate (1.02 g, 7.36 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2,2-dimethylpropane-3,1-diyl))dicarbamate as a colorless oil (0.63 g, 1.38 mmol, 56.3%). UPLC/ELSD: RT=0.46 min. MS (ES): m/z (MH⁺) 459.7 for C₂₄H₅₀N₄O₄. ¹H NMR (300 MHz, CDCl₃) δ: ppm 5.84 (m, 2H), 2.82 (d, 4H, J=6 Hz), 2.39 (br. s, 4H), 2.22 (s, 4H), 1.33 (br. m, 4H), 1.24 (s, 18H), 0.70 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate

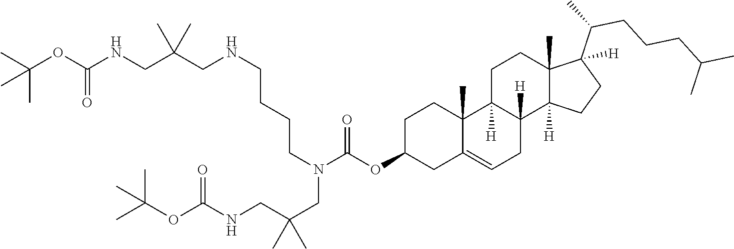

To a solution of di-tert-butyl ((butane-1,4-diylbis(azanediyl))bis(2,2-dimethylpropane-3,1-diyl))dicarbamate (0.66 g, 1.43 mmol) in dry toluene (10 mL) set stirring under nitrogen was added triethylamine (0.55 mL, 3.90 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.72 g, 1.30 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate as a colorless oil (0.39 g, 0.45 mmol, 34.7%). UPLC/ELSD: RT=2.77 min. MS (ES): m/z (MH⁺) 872.3 for C₅₂H₉₄N₄O₆. ¹H NMR (300 MHz, CDCl₃) δ: ppm 5.38 (m, 1H), 5.09 (m, 1H), 4.23 (m, 1H), 2.92 (br. m, 2H), 2.72 (m, 6H), 2.27 (m, 2H), 2.10 (m, 4H), 1.70 (m, 5H), 1.28 (br. m, 8H), 1.14 (s, 21H), 1.05 (m, 4H), 0.85 (m, 8H), 0.74 (s, 7H), 0.60 (br. m, 22H), 0.39 (s, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride

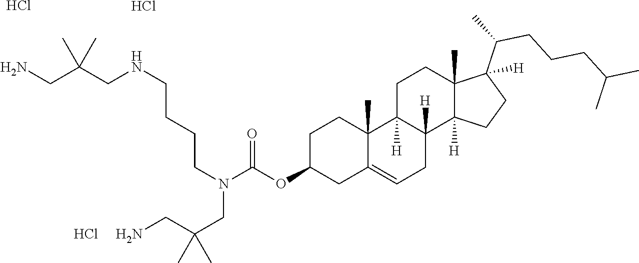

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-dimethylpropyl)(4-((3-((tert-butoxycarbonyl)amino)-2,2-dimethylpropyl)amino)butyl)carbamate (0.39 g, 0.45 mmol) in isopropanol (10 mL) stirring under nitrogen was added hydrochloric acid (5 N in isopropanol, 0.90 mL, 4.51 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-dimethylpropyl)(4-((3-amino-2,2-dimethylpropyl)amino)butyl)carbamate trihydrochloride as a white solid SA77 (0.21 g, 0.26 mmol, 58.0%). UPLC/ELSD: RT=1.49 min. MS (ES): m/z (MH⁺) 781.5 for C₄₂H₈₁C₃N₄O₂. ¹H NMR (300 MHz, CDCl₃) δ: ppm 5.42 (m, 1H), 4.51 (br. m, 1H), 3.33 (br. m, 5H), 3.08 (d, 6H, J=9 Hz), 2.73 (m, 2H), 2.42 (d, 2H, J=6 Hz), 1.75 (br. m, 16H), 1.39 (br. m, 4H), 1.22 (br. m, 11H), 1.09 (br. m, 11H), 0.97 (d, 4H, J=6 Hz), 0.90 (d, 6H, J=6 Hz), 0.74 (s, 3H).

### Y. Compound SA78: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

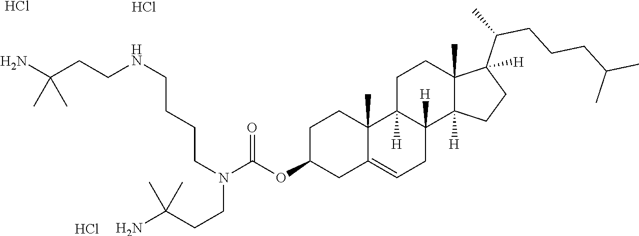

### Step 1: tert-butyl (2-methyl-4-((4-nitrophenyl)sulfonamido)butan-2-yl)carbamate

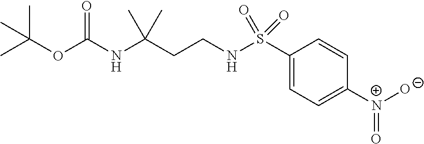 [SMILES: CC(C)(CCNS(=O)(=O)c1ccc([N+](=O)[O-])cc1)NC(=O)OC(C)(C)C]

To a solution of tert-butyl (4-amino-2-methylbutan-2-yl)carbamate (1.00 g, 4.94 mmol) in dry DCM (15 mL) stirring under nitrogen was added triethylamine (0.83 mL, 5.93 mmol). The solution was cooled to 0° C., and then a solution of 4-nitrobenzenesulfonyl chloride (1.20 g, 5.44 mmol) in 5 mL dry DCM was added dropwise over 30 minutes. The reaction was allowed to proceed at 0° C. for an hour and then at room temperature for an additional three hours. Then the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-methyl-4-((4-nitrophenyl)sulfonamido)butan-2-yl)carbamate as a white solid (1.86 g, 4.79 mmol, 96.9%). UPLC/ELSD: RT=0.69 min. MS (ES): m/z (MH⁺) 388.4 for C₁₆H₂₅N₃O₆S. ¹H NMR (300 MHz, CDCl₃) δ: ppm 8.10 (m, 1H), 7.84 (m, 1H), 7.75 (m, 2H), 5.46 (br. s, 1H), 4.47 (s, 1H), 3.15 (q, 2H), 1.94 (t, 2H), 1.39 (s, 9H), 1.23 (s, 6H).

### Step 2: di-tert-butyl ((butane-1,4-diylbis(azanediyl))bis(2-methylbutane-4,2-diyl))dicarbamate

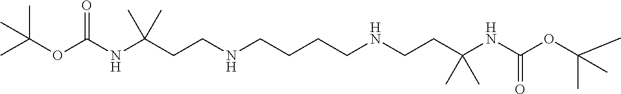 [SMILES: CC(C)(CCNCCCCNCCC(C)(C)NC(=O)OC(C)(C)C)NC(=O)OC(C)(C)C]

To a solution of tert-butyl (2-methyl-4-((4-nitrophenyl)sulfonamido)butan-2-yl)carbamate (1.86 g, 4.79 mmol) in dry DMF (20 mL) set stirring under nitrogen was added potassium carbonate (1.92 g, 13.92 mmol) and 1,4-diiodobutane (0.30 mL, 2.28 mmol). The solution was heated to 40° C. and allowed to proceed overnight. The following morning, benzyl bromide (0.23 mL, 1.89 mmol) was added, and the reaction was allowed to proceed at room temperature for 24 h. Then, thiophenol (0.90 mL, 8.78 mmol), potassium carbonate (0.95 g, 6.84 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-methylbutane-4,2-diyl))dicarbamate as a colorless oil (0.57 g, 1.24 mmol, 54.3%). UPLC/ELSD: RT=0.46 min. MS (ES): m/z (MH⁺) 459.7 for C₂₄H₅₀N₄O₄. ¹H NMR (300 MHz, CDCl₃) δ: ppm 5.94 (m, 2H), 2.61 (m, 8H), 1.92 (br. m, 2H), 1.61 (m, 4H), 1.45 (br. m, 4H), 1.32 (s, 18H), 1.21 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate

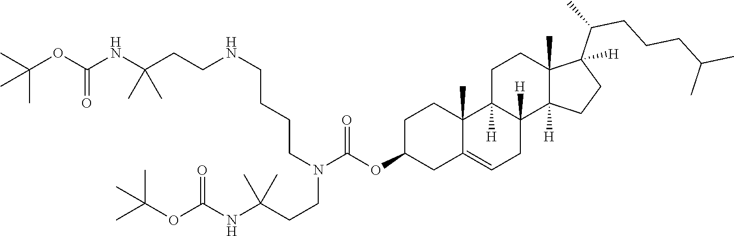

To a solution of di-tert-butyl ((butane-1,4-diylbis(azanediyl))bis(2-methylbutane-4,2-diyl))dicarbamate (0.68 g, 1.48 mmol) in dry toluene (10 mL) stirring under nitrogen was added triethylamine (0.57 mL, 4.03 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.74 g, 1.34 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate as a colorless oil (0.57 g, 0.65 mmol, 48.3%). UPLC/ELSD: RT=2.65 min. MS (ES): m/z (MH⁺) 872.3 for C₅₂H₉₄N₄O₆. ¹H NMR (300 MHz, CDCl₃) δ: ppm 5.99 (m, 1H), 5.27 (m, 1H), 4.40 (m, 2H), 3.11 (br. m, 4H), 2.59 (t, 2H), 2.50 (t, 2H), 2.25 (m, 2H), 1.77 (m, 7H), 1.58 (m, 2H), 1.44 (br. m, 12H), 1.32 (s, 18H), 1.20 (d, 16H, J=9 Hz), 1.01 (m, 9H), 0.92 (s, 6H), 0.82 (d, 4H, J=6 Hz), 0.75 (d, 6H, J=9 Hz), 0.57 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

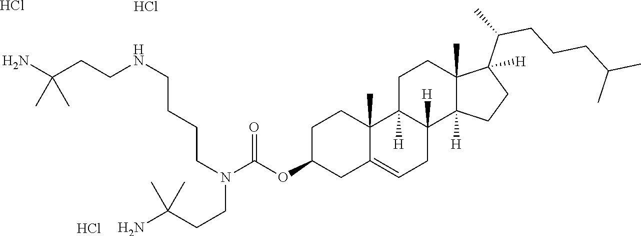

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate (0.57 g, 0.65 mmol) in isopropanol (10 mL) set stirring under nitrogen was added hydrochloric acid (5 N in isopropanol, 1.30 mL, 6.50 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give SA78 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride as a white solid (0.35 g, 0.44 mmol, 67.2%). UPLC/ELSD: RT=1.50 min. MS (ES): m/z (MH⁺) 781.5 for C₄₂H₈₁C₃N₄O₂. ¹H NMR (300 MHz, CDCl₃) δ: ppm 5.43 (m, 1H), 4.44 (br. m, 1H), 3.33 (br. m, 5H), 3.15 (br. m, 3H), 2.38 (m, 2H), 2.16 (br. m, 8H), 1.74 (br. m, 10H), 1.43 (br. m, 14H), 1.17 (d, 9H, J=6 Hz), 1.08 (br. m, 5H), 0.98 (d, 4H, J=6 Hz), 0.90 (d, 6H, J=6 Hz), 0.74 (s, 3H).

### Z. Compound SA79: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

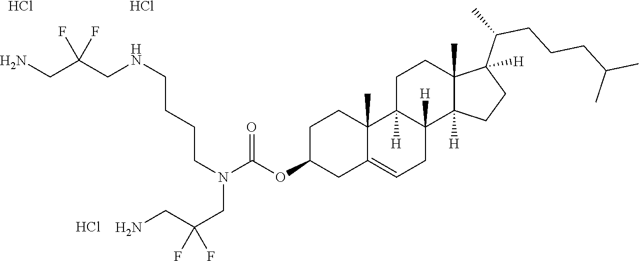

### Step 1: tert-butyl (2,2-difluoro-3-((4-nitrophenyl)sulfonamido)propyl)carbamate

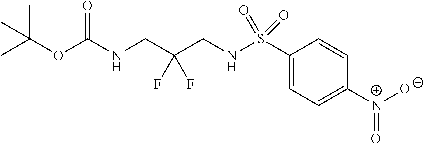 [SMILES: CC(C)(C)OC(=O)NCC(F)(F)CNS(=O)(=O)c1ccc([N+](=O)[O-])cc1]

To a solution of tert-butyl (3-amino-2,2-difluoropropyl)carbamate (0.95 g, 4.52 mmol) in dry DCM (15 mL) set stirring under nitrogen was added triethylamine (0.76 mL, 5.42 mmol). The solution was cooled to 0° C., and then a solution of 4-nitrobenzenesulfonyl chloride (1.10 g, 4.97 mmol) in 5 mL dry DCM was added dropwise over 30 minutes. The reaction was allowed to proceed at 0° C. for an hour, and then at room temperature for an additional three hours. Then the mixture was diluted with an additional 10 mL DCM, washed with 1 M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2,2-difluoro-3-((4-nitrophenyl)sulfonamido)propyl)carbamate as a white solid (1.66 g, 4.19 mmol, 92.6%). UPLC/ELSD: RT=0.61 min. MS (ES): m/z (MH⁺) 396.4 for C₁₄H₁₉F₂N₃O₆S. ¹H NMR (300 MHz, CDCl₃) δ: ppm 8.15 (m, 1H), 7.87 (m, 1H), 7.73 (m, 2H), 6.68 (br. s, 1H), 5.02 (br. s, 1H), 3.55 (br. m, 4H), 1.45 (s, 9H).

### Step 2: di-tert-butyl ((butane-1,4-diylbis(azanediyl))bis(2,2-difluoropropane-3,1-diyl))dicarbamate

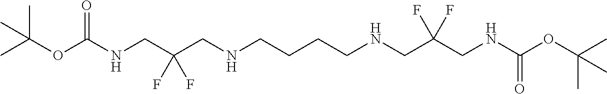 [SMILES: CC(C)(C)OC(=O)NCC(F)(F)CNCCCCNCC(F)(F)CNC(=O)OC(C)(C)C]

To a solution of tert-butyl (2,2-difluoro-3-((4-nitrophenyl)sulfonamido)propyl)carbamate (1.65 g, 4.18 mmol) in dry DMF (20 mL) set stirring under nitrogen was added potassium carbonate (1.68 g, 12.14 mmol) and 1,4-diiodobutane (0.26 mL, 1.99 mmol). The solution was heated to 40° C. and allowed to proceed overnight. The following morning, benzyl bromide (0.20 mL, 1.65 mmol) was added, and the reaction was allowed to proceed at room temperature for 24 h. Then thiophenol (0.78 mL, 7.67 mmol), potassium carbonate (0.83 g, 5.97 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2,2-difluoropropane-3,1-diyl))dicarbamate as a colorless oil (0.50 g, 1.07 mmol, 53.3%). UPLC/ELSD: RT=0.39 min. MS (ES): m/z (MH⁺) 475.5 for C₂₀H₃₈F₄N₄O₄. ¹H NMR (300 MHz, CDCl₃) δ: ppm 5.14 (m, 2H), 3.25 (m, 4H), 2.62 (m, 4H), 2.33 (br. m, 4H), 1.18 (br. m, 4H), 1.12 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate

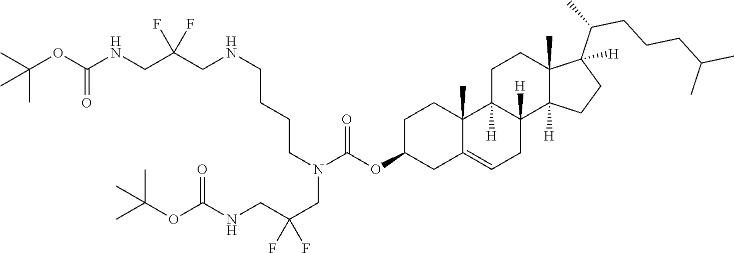

To a solution of di-tert-butyl ((butane-1,4-diylbis(azanediyl))bis(2,2-difluoropropane-3,1-diyl))dicarbamate (0.64 g, 1.35 mmol) in dry toluene (10 mL) stirring under nitrogen was added triethylamine (0.52 mL, 3.68 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.68 g, 1.23 mmol) was added, and the solution was heated to 90° C. and allowed to proceed overnight. The following morning, the reaction mixture was allowed to cool to room temperature, and the solution was washed with water (3×10 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate as a colorless oil (0.12 g, 0.13 mmol, 10.7%). UPLC/ELSD: RT=2.63 min. MS (ES): m/z (MH⁺) 888.2 for C₄₈H₈₂F₄N₄O₆. ¹H NMR (300 MHz, CDCl₃) δ: ppm 5.61 (m, 1H), 5.31 (m, 1H), 4.98 (m, 1H), 4.55 (br. m, 1H), 3.63 (br. m, 5H), 3.32 (m, 2H), 2.97 (t, 2H), 2.69 (t, 2H), 2.36 (m, 2H), 2.05 (br. m, 5H), 1.60 (br. m, 5H), 1.46 (s, 21H), 1.15 (m, 6H), 1.04 (s, 5H), 0.93 (d, 3H, J=6 Hz), 0.89 (d, 5H, J=6 Hz), 0.69 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

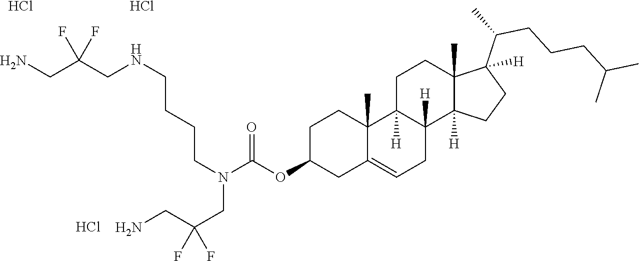

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate (0.12 g, 0.13 mmol) in isopropanol (10 mL) stirring under nitrogen was added hydrochloric acid (5 N in isopropanol, 0.26 mL, 1.31 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride as a white solid SA79 (0.05 g, 0.06 mmol, 42.8%). UPLC/ELSD: RT=1.52 min. MS (ES): m/z (MH⁺) 797.4 for C₃₈H₆₉C₁₃F₄N₄O₂. ¹H NMR (300 MHz, CDCl₃) δ: ppm 5.43 (m, 1H), 4.49 (br. m, 1H), 3.94 (br. m, 7H), 3.77 (br. m, 4H), 3.33 (m, 3H), 3.22 (m, 2H), 2.42 (m, 2H), 1.76 (br. m, 23H), 1.18 (d, 12H, J=6 Hz), 1.08 (br. m, 6H), 0.98 (d, 4H, J=9 Hz), 0.91 (d, 6H, J=6 Hz), 0.75 (s, 3H).

### AA. Compound SA81: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride

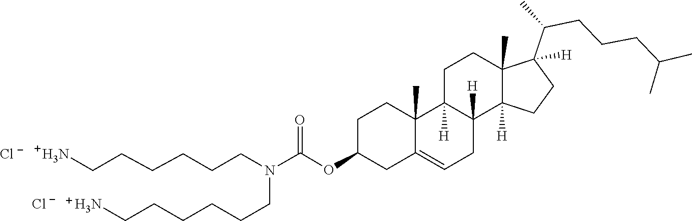

### Step 1: tert-Butyl N-{6-[benzyl({6-[(tert-butoxycarbonyl)amino]hexyl})amino]hexyl}carbamate

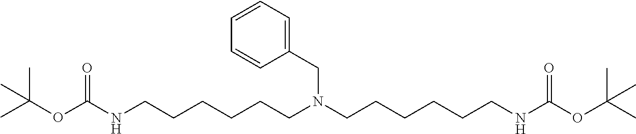 [SMILES: CC(C)(C)OC(=O)NCCCCCCN(CCCCCCNC(=O)OC(C)(C)C)Cc1ccccc1]

To a suspension of tert-butyl N-(6-bromohexyl)carbamate (2.694 g, 9.613 mmol), potassium carbonate (1.898 g, 13.73 mmol), and potassium iodide (0.152 g, 0.915 mmol) in dimethylformamide (7.5 mL) was added benzylamine (0.50 mL, 4.6 mmol). The reaction mixture stirred at 50° C. and was monitored by LCMS. At 23.5 h, the reaction mixture was cooled to rt and then diluted with methyl tert-butyl ether (150 mL). The diluted mixture was washed with water (4×) and brine, dried over Na₂SO₄, and concentrated. The crude material was purified via silica gel chromatography (20-70% methyl tert-butyl ether in hexanes) to afford tert-butyl N-{6-[benzyl({6-[(tert-butoxycarbonyl)amino]hexyl})amino]hexyl}carbamate (2.144 g, 4.239 mmol, 92.6%) as a clear oil. UPLC/ELSD: RT=0.87 min. MS (ES): m/z=506.70 [M+H]⁺ for C₂₉H₅₁N₃O₄; ¹H NMR (300 MHz, CDCl₃): δ 7.17-7.37 (m, 5H), 4.50 (br. s, 2H), 3.52 (s, 2H), 3.08 (dt, 4H, J=6.5, 6.2 Hz), 2.37 (t, 4H, J=7.2 Hz), 1.37-1.51 (m, 8H), 1.44 (s, 18H), 1.22-1.33 (m, 8H).

### Step 2: tert-Butyl N-[6-({6-[(tert-butoxycarbonyl)amino]hexyl}amino)hexyl]carbamate

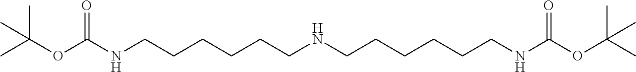 [SMILES: CC(C)(C)OC(=O)NCCCCCCNCCCCCCNC(=O)OC(C)(C)C]

Tert-butyl N-{6-[benzyl({6-[(tert-butoxycarbonyl)amino]hexyl})amino]hexyl}carbamate (2.12 g, 4.192 mmol) and 10% Pd/C (0.892 g, 0.419 mmol) were combined in ethanol (35 mL) and then stirred under a balloon of H₂ at rt. The reaction was monitored by TLC. At 19 h, the reaction mixture was filtered through a pad of Celite rinsing with ethyl acetate. The filtrate was concentrated, taken up in ethyl acetate, filtered through a 0.45 μm PTFE frit, and concentrated to afford tert-butyl N-[6-({6-[(tert-butoxycarbonyl)amino]hexyl}amino)hexyl]carbamate (1.537 g, 3.698 mmol, 88.2%) as an off-white solid. UPLC/ELSD: RT=0.62 min. MS (ES): m/z=416.60 [M+H]⁺ for C₂₂H₄₅N₃O₄; ¹H NMR (300 MHz, CDCl₃): δ 4.51 (br. s, 2H), 3.10 (dt, 4H, J=6.4, 6.4 Hz), 2.57 (t, 4H, J=7.1 Hz), 1.13-1.57 (br. m, 17H), 1.44 (s, 18H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate

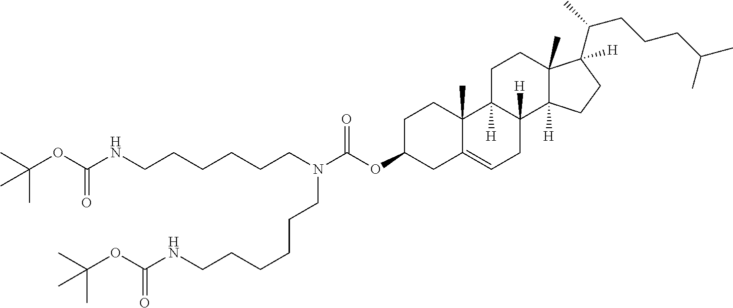

Cholesterol 4-nitrophenyl carbonate (0.200 g, 0.362 mmol), tert-butyl N-[6-({6-[(tert-butoxycarbonyl)amino]hexyl}amino)hexyl]carbamate (0.188 g, 0.453 mmol), and triethylamine (0.15 mL, 1.08 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×25 mL). The organics were passed through a hydrophobic frit, dried over Na₂SO₄, and concentrated. The crude material was purified via silica gel chromatography (0-50% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.286 g, 0.345 mmol, 95.3%) as a clear oil.

UPLC/ELSD: RT=3.53 min. MS (ES): m/z=728.94 [(M+H)—(CH₃)₂C═CH₂ —CO₂]⁺ for C₅₀H₈₉N₃O₆; ¹H NMR (300 MHz, CDCl₃): δ 5.33-5.42 (m, 1H), 4.41-4.64 (m, 3H), 2.99-3.28 (m, 8H), 2.20-2.41 (m, 2H), 1.75-2.08 (m, 5H), 0.93-1.67 (br. m, 37H), 1.44 (s, 18H), 1.02 (s, 3H), 0.91 (d, 3H, J=6.4 Hz), 0.87 (d, 3H, J=6.6 Hz), 0.86 (d, 3H, J=6.5 Hz), 0.68 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride

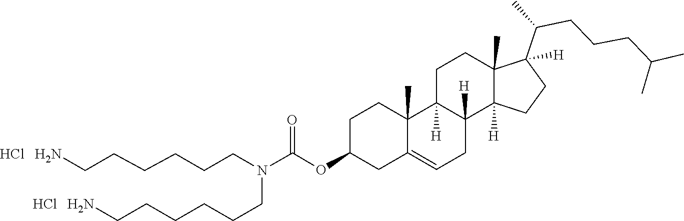

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.280 g, 0.338 mmol) in isopropanol (3.5 mL) was added 5-6 N HCl in isopropanol (0.52 mL). The reaction mixture stirred at 40° C. and was monitored by LCMS. At 18 h, the reaction mixture was cooled to rt and then acetonitrile (10.5 mL) was added. The suspension was cooled to 0° C. in an ice bath. Solids were then collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(6-aminohexyl)carbamate dihydrochloride (0.179 g, 0.251 mmol, 74.4%) as a white solid).

UPLC/ELSD: RT=2.09 min. MS (ES): m/z=335.49 [(M+2H)+CH₃CN]²⁺ for C₄₀H₇₃N₃O₂; ¹H NMR (300 MHz, CDCl₃): δ 5.36-5.44 (m, 1H), 4.33-4.48 (m, 1H), 3.25 (t, 4H, J=7.2 Hz), 2.92 (t, 4H, J=7.6 Hz), 2.24-2.39 (m, 2H), 1.79-2.12 (m, 5H), 0.97-1.73 (br. m, 37H), 1.05 (s, 3H), 0.95 (d, 3H, J=6.4 Hz), 0.88 (d, 3H, J=6.5 Hz), 0.88 (d, 3H, J=6.6 Hz), 0.73 (s, 3H).

### AB. Compound SA82: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-aminohexyl)carbamate dihydrochloride

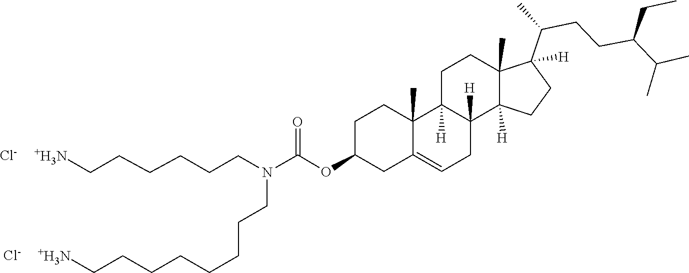

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate

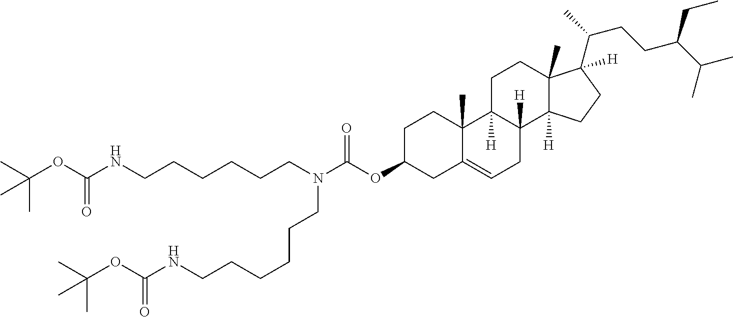

β-Sitosterol 4-nitrophenyl carbonate (0.200 g, 0.345 mmol), tert-butyl N-[6-({6-[(tert-butoxycarbonyl)amino]hexyl}amino)hexyl]carbamate (0.179 g, 0.431 mmol), triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (ca. 30 mL) and washed with 5% aq. NaHCO₃ solution (3×25 mL). The combined washes were extracted with dichloromethane (25 mL). The combined organics were passed through a hydrophobic frit, dried over Na₂SO₄, and concentrated. The crude material was purified via silica gel chromatography (0-40% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.259 g, 0.302 mmol, 87.7%) as a clear oil.

UPLC/ELSD: RT=3.63 min. MS (ES): m/z=857.26 [M+H]⁺ for C₅₂H₉₃N₃O₆; ¹H NMR (300 MHz, CDCl₃): δ 5.33-5.42 (m, 1H), 4.41-4.61 (m, 3H), 3.02-3.28 (m, 8H), 2.19-2.42 (m, 2H), 1.76-2.08 (m, 5H), 0.88-1.73 (br. m, 38H), 1.44 (s, 18H), 1.02 (s, 3H), 0.92 (d, 3H, J=6.4 Hz), 0.79-0.89 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-aminohexyl)carbamate dihydrochloride

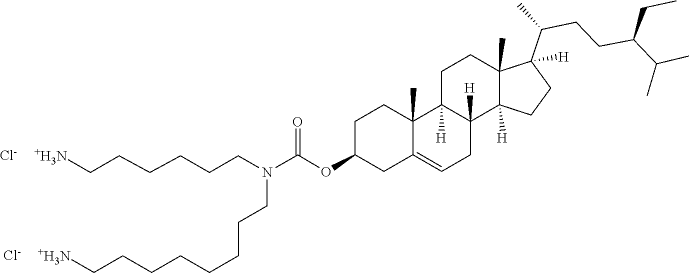

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(6-((tert-butoxycarbonyl)amino)hexyl)carbamate (0.256 g, 0.299 mmol) in isopropanol (3.2 mL) was added 5-6 N HCl in isopropanol (0.45 mL). The reaction mixture stirred at 40° C. and was monitored by LCMS. At 18 h, the reaction mixture was cooled to rt and then acetonitrile (9.6 mL) was added. The suspension was cooled to 0° C. in an ice bath, and solids were collected by vacuum filtration rinsing with cold 3:1 acetonitrile/isopropanol to afford (3S,8S,9S,10R,13R,14S,17R)-17-((2R,5R)-5-ethyl-6-methylheptan-2-yl)-10,13-dimethyl-2,4,7,8,9,10,11,12,13,14,15,16,17-tetradecahydro-1H-cyclopenta[a]phenanthren-3-yl bis(6-aminohexyl)carbamate dihydrochloride (0.167 g, 0.216 mmol, 72.1%) as a white solid. UPLC/ELSD: RT=2.23 min. MS (ES): m/z=349.94 [(M+2H)+CH₃CN]²⁺ for C₄₂H₇₇N₃O₂; ¹H NMR (300 MHz, CD₃OD): δ 5.36-5.54 (m, 1H), 4.33-4.48 (m, 1H), 3.25 (t, 4H, J=7.1 Hz), 2.92 (t, 4H, J=7.6 Hz), 2.25-2.39 (m, 2H), 1.79-2.13 (m, 5H), 0.91-1.76 (br. m, 38H), 1.05 (s, 3H), 0.96 (d, 3H, J=6.4 Hz), 0.81-0.91 (m, 9H), 0.73 (s, 3H).

### AC. Compound SA83: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

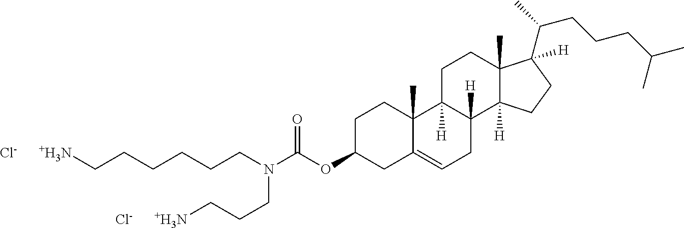

### Step 1: tert-Butyl N-[3-(2-nitrobenzenesulfonamido)propyl]carbamate

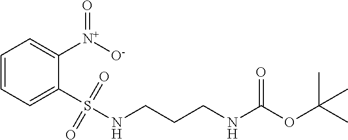 [SMILES: CC(C)(C)OC(=O)NCCCNS(=O)(=O)c1ccccc1[N+](=O)[O-]]

To a stirred solution of tert-butyl N-(3-aminopropyl)carbamate (1.50 g, 8.35 mmol) and triethylamine (1.50 mL, 10.7 mmol) in dichloromethane (40 mL) cooled to 0° C. in an ice bath, was added a solution of 2-nitrobenzenesulfonyl chloride (2.00 g, 8.75 mmol) in dichloromethane (10 mL) dropwise via addition funnel over 15 min. After this time, the reaction mixture was allowed to slowly warm to rt while stirring. The reaction was monitored by TLC. At 23 h, the reaction mixture was diluted with dichloromethane (50 mL) and then washed with 5% aq. citric acid. The organics were passed through a hydrophobic frit, dried over Na₂SO₄, and concentrated. The crude material was purified via silica gel chromatography (20-50% ethyl acetate in hexanes) to afford tert-butyl N-[3-(2-nitrobenzenesulfonamido)propyl]carbamate (2.611 g, 7.265 mmol, 87.0%) as a viscous, light yellow oil. UPLC/ELSD: RT=0.54 min. MS (ES): m/z=304.14 [(M+H)—(CH₃)₂C═CH₂]⁺ for C₁₄H₂₁N₃O₆S; ¹H NMR (300 MHz, CDCl₃): δ 8.09-8.17 (m, 1H), 7.81-7.89 (m, 1H), 7.68-7.77 (m, 2H), 5.86 (br. s, 1H), 4.66 (br. s, 1H), 3.21 (dt, 2H, J=6.2, 6.2 Hz), 3.15 (dt, 2H, J=6.4, 6.4 Hz), 1.63-1.76 (m, 2H), 1.42 (s, 9H).

### Step 2: Tert-butyl N-[3-(N-{6-[(tert-butoxycarbonyl)amino]hexyl}2-nitrobenzenesulfonamido)propyl]carbamate

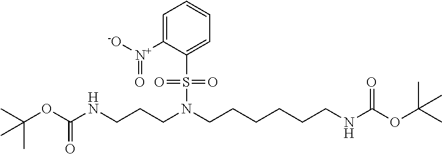

To a stirred mixture of tert-butyl N-[3-(2-nitrobenzenesulfonamido)propyl]carbamate (1.000 g, 2.782 mmol), potassium carbonate (0.769 g, 5.56 mmol), and potassium iodide (0.046 g, 0.28 mmol) in dimethylformamide (15 mL) was added a solution of tert-butyl N-(6-bromohexyl)carbamate (0.858 g, 3.06 mmol) in dimethylformamide (1.0 mL). The reaction mixture stirred at rt and was monitored by LCMS. At 20.3 h, the reaction mixture was heated to 50° C. At 25 h, the reaction mixture was cooled to rt. The reaction mixture was diluted with methyl tert-butyl ether and water. The layers were separated, and the organics were washed with water (4×) and brine, dried over Na₂SO₄, and concentrated. The crude material was purified via silica gel chromatography (10-85% methyl tert-butyl ether in hexanes) to afford tert-butyl N-[3-(N-{6-[(tert-butoxycarbonyl)amino]hexyl}2-nitrobenzenesulfonamido)propyl]carbamate (1.399 g, 2.504 mmol, 90.0%) as a clear oil. UPLC/ELSD: RT=1.43 min. MS (ES): m/z=403.26 [(M+H)-2[(CH₃)₂C═CH₂]—CO₂]⁺ for C₂₅H₄₂N₄O₈S; ¹H NMR (300 MHz, CDCl₃): δ 7.96-8.04 (m, 1H), 7.58-7.73 (m, 3H), 4.84 (br. s, 1H), 4.50 (br. s, 1H), 3.35 (t, 2H, J=7.0 Hz), 3.25 (t, 2H, J=7.6 Hz), 3.15 (td, 2H, J=6.3, 6.2 Hz), 3.06 (td, 2H, J=6.7, 6.5 Hz), 1.68-1.79 (m, 2H), 1.34-1.60 (m, 4H), 1.44 (s, 18H), 1.20-1.33 (m, 4H).

### Step 3: Tert-butyl N-[3-({6-[(tert-butoxycarbonyl)amino]hexyl}amino)propyl]carbamate

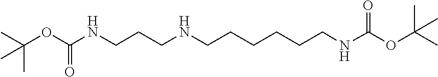 [SMILES: CC(C)(C)OC(=O)NCCCCCCNCCCNC(=O)OC(C)(C)C]

Tert-butyl N-[3-(N-{6-[(tert-butoxycarbonyl)amino]hexyl}2-nitrobenzenesulfonamido)propyl]carbamate (1.391 g, 2.490 mmol), potassium carbonate (1.032 g, 7.469 mmol), and thiophenol (0.39 mL, 3.82 mmol) were combined in dimethylformamide (20 mL). The reaction mixture stirred at rt and was monitored by LCMS. At 18 h, the reaction mixture was filtered through a pad of Celite rinsing with methyl tert-butyl ether. The filtrate was washed with saturated aq. NaHCO₃ solution, water (3×), and brine. The organics were dried over Na₂SO₄ and concentrated. The crude material was purified via silica gel chromatography (0-20% (5% conc. aq. NH₄OH in methanol) in dichloromethane) to afford tert-butyl N-[3-({6-[(tert-butoxycarbonyl)amino]hexyl}amino)propyl]carbamate (0.889 g, 2.38 mmol, 95.6%) as a white solid. UPLC/ELSD: RT=0.42 min. MS (ES): m/z=374.38 [M+H]⁺ for C₁₉H₃₉N₃O₄; ¹H NMR (300 MHz, CDCl₃): δ 5.15 (br. s, 1H), 4.52 (br. s, 1H), 3.19 (dt, 2H, J=5.9, 5.9 Hz), 3.10 (dt, 2H, J=6.4, 6.4 Hz), 2.66 (t, 2H, J=6.6 Hz), 2.57 (t, 2H, J=7.0 Hz), 1.59-1.71 (m, 2H), 1.24-1.55 (m, 9H), 1.44 (s, 18H).

### Step 4: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate

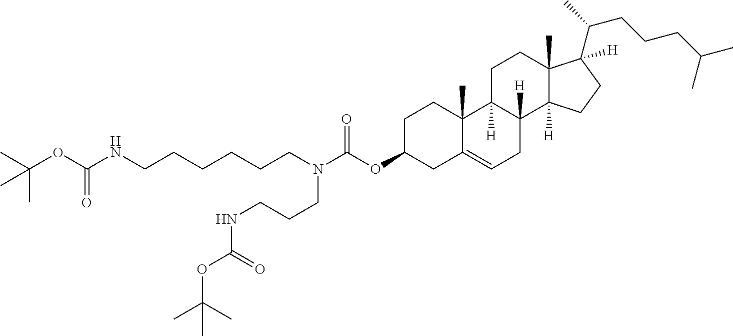

Cholesterol 4-nitrophenyl carbonate (0.200 g, 0.362 mmol), tert-butyl N-[3-({6-[(tert-butoxycarbonyl)amino]hexyl}amino)propyl]carbamate (0.169 g, 0.453 mmol), and triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×30 mL). The organics were passed through a hydrophobic frit, dried over Na₂SO₄, and concentrated. The crude material was purified via silica gel chromatography (20-50% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.240 g, 0.305 mmol, 84.2%) as a clear oil. UPLC/ELSD: RT=3.43 min. MS (ES): m/z=687.36 [(M+H)—(CH₃)₂C═CH₂—CO₂]⁺ for C₄₇H₈₃N₃O₆; ¹H NMR (300 MHz, CDCl₃): δ 5.17-5.44 (m, 2H), 4.40-4.86 (m, 2H), 2.98-3.40 (br. m, 8H), 2.19-2.44 (m, 2H), 1.74-2.10 (m, 5H), 0.93-1.73 (br. m, 31H), 1.44 (s, 18H), 1.02 (s, 3H), 0.91 (d, 3H, J=6.4 Hz), 0.86 (d, 3H, J=6.6 Hz), 0.86 (d, 3H, J=6.6 Hz), 0.68 (s, 3H).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

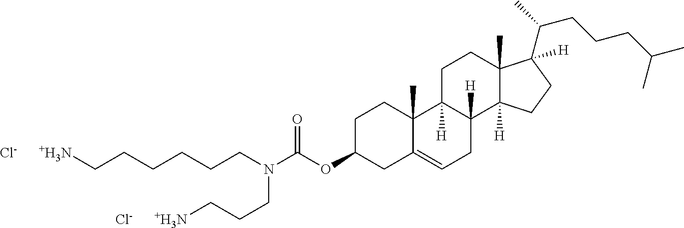

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.234 g, 0.298 mmol) in isopropanol (2.8 mL) was added 5-6 N HCl in isopropanol (0.42 mL). The reaction mixture stirred at 40° C. and was monitored by LCMS. At 17 h, the reaction mixture was cooled to rt and then acetonitrile (8.4 mL) was added. The suspension stirred at rt for 1 h and then solids were collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride (0.177 g, 0.264 mmol, 88.8%) as a white solid. UPLC/ELSD: RT=1.98 min. MS (ES): m/z=314.63 [(M+2H)+CH₃CN]²⁺ for C₃₇H₆₇N₃O₂; ¹H NMR (300 MHz, CD₃OD): δ 5.35-5.44 (m, 1H), 4.36-4.52 (m, 1H), 3.37 (t, 2H, J=6.8 Hz), 3.28 (t, 2H, J=7.6 Hz), 2.88-2.99 (m, 4H), 2.26-2.46 (m, 2H), 1.78-2.14 (m, 7H), 0.98-1.75 (br. m, 29H), 1.06 (s, 3H), 0.95 (d, 3H, J=6.4 Hz), 0.88 (d, 3H, J=6.6 Hz), 0.88 (d, 3H, J=6.6 Hz), 0.73 (s, 3H).

### AD. Compound SA84: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

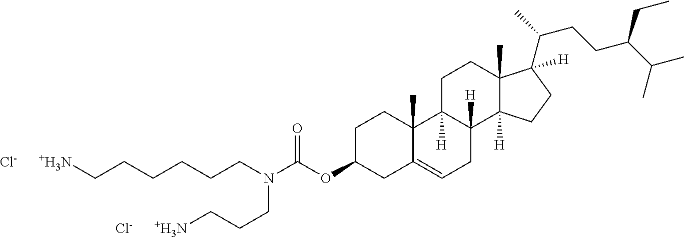

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate

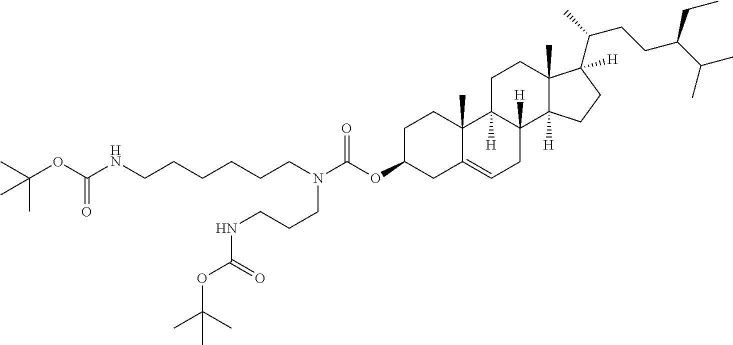

β-Sitosterol 4-nitrophenyl carbonate (0.200 g, 0.345 mmol), tert-butyl N-[3-({6-[(tert-butoxycarbonyl)amino]hexyl}amino)propyl]carbamate (0.161 g, 0.431 mmol), and triethylamine (0.15 mL, 1.1 mmol) were combined in toluene (3.5 mL). The reaction mixture stirred at 90° C. and was monitored by LCMS. At 17 h, the reaction mixture was cooled to rt, diluted with dichloromethane (30 mL), and washed with 5% aq. NaHCO₃ solution (3×30 mL). The organics were passed through a hydrophobic frit, dried over Na₂SO₄, and concentrated. The crude material was purified via silica gel chromatography (20-50% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.218 g, 0.268 mmol, 77.6%) as a clear oil. UPLC/ELSD: RT=3.55 min. MS (ES): m/z=715.12 [(M+H)—(CH₃)₂C═CH₂—CO₂]⁺ for C₄₉H₈₇N₃O₆; ¹H NMR (300 MHz, CDCl₃): δ 5.13-5.44 (m, 2H), 4.35-4.88 (m, 2H), 2.98-3.40 (br. m, 8H), 2.20-2.45 (m, 2H), 1.76-2.09 (m, 5H), 0.88-1.75 (br. m, 32H), 1.44 (s, 18H), 1.02 (s, 3H), 0.92 (d, 3H, J=6.4 Hz), 0.78-0.89 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride

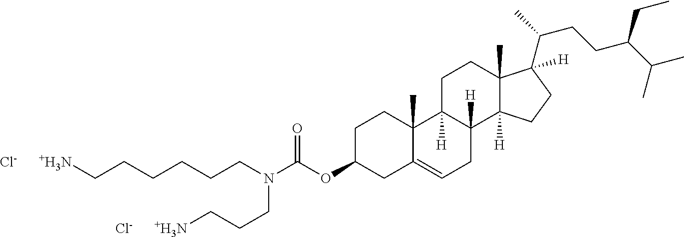

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-((tert-butoxycarbonyl)amino)hexyl)(3-((tert-butoxycarbonyl)amino)propyl)carbamate (0.213 g, 0.262 mmol) in isopropanol (2.6 mL) was added 5-6 N HCl in isopropanol (0.38 mL). The reaction mixture stirred at 40° C. and was monitored by LCMS. At 17 h, the reaction mixture was cooled to rt, and then acetonitrile (7.8 mL) was added. The suspension stirred at rt for 1 h, and then solids were collected by vacuum filtration rinsing with cold 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (6-aminohexyl)(3-aminopropyl)carbamate dihydrochloride (0.169 g, 0.232 mmol, 88.8%) as a white solid. UPLC/ELSD: RT=2.17 min. MS (ES): m/z=328.70 [(M+2H)+CH₃CN]²⁺ for C₃₉H₇₁N₃O₂; ¹H NMR (300 MHz, CD₃OD): δ 5.36-5.45 (m, 1H), 4.36-5.53 (m, 1H), 3.37 (t, 2H, J=6.6 Hz), 3.28 (t, 2H, J=7.5 Hz), 2.88-2.98 (m, 4H), 2.26-2.43 (m, 2H), 1.80-2.12 (m, 7H), 0.91-1.77 (br. m, 30H), 1.06 (s, 3H), 0.96 (d, 3H, J=6.4 Hz), 0.82-0.91 (m, 9H), 0.73 (s, 3H).

### AE. Compound SA87: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride

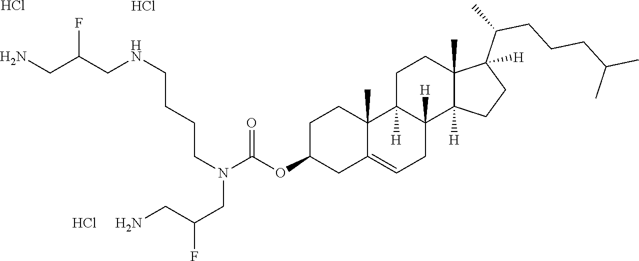

### Step 1: tert-butyl (2-fluoro-3-((2-nitrophenyl)sulfonamido)propyl)carbamate

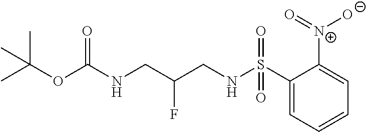 [SMILES: CC(C)(C)OC(=O)NCC(F)CNS(=O)(=O)c1ccccc1[N+](=O)[O-]]

To a solution of tert-butyl (3-amino-2-fluoropropyl)carbamate (1.00 g, 5.20 mmol) in dry DCM (15 mL) set stirring under nitrogen was added triethylamine (0.87 mL, 6.24 mmol). The solution was cooled to 0° C. and then a solution of 2-nitrobenzenesulfonyl chloride (1.27 g, 5.72 mmol) in 5 mL dry DCM was added dropwise over 30 minutes. The reaction was allowed to proceed at 0° C. for an hour and then at room temperature for an additional three hours. The mixture was then diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (2-fluoro-3-((2-nitrophenyl)sulfonamido)propyl)carbamate as a white solid (1.93 g, 5.14 mmol, 98.7%). UPLC/ELSD: RT=1.76 min. MS (ES): m/z (MH⁺) 378.4 for C₁₄H₂₀FN₃O₆S. ¹H NMR (300 MHz, CDCl₃) δ: ppm 8.15 (m, 1H), 7.87 (m, 1H), 7.77 (m, 2H), 6.14 (br. s, 1H), 5.31 (m, 1H), 4.71 (m, 1H), 4.55 (m, 1H), 3.39 (m, 4H), 1.44 (s, 9H).

### Step 2: di-tert-butyl ((butane-1,4-diylbis(azanediyl))bis(2-fluoropropane-3,1-diyl))dicarbamate

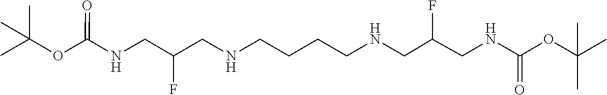 [SMILES: CC(C)(C)OC(=O)NCC(F)CNCCCCNCC(F)CNC(=O)OC(C)(C)C]

To a solution of tert-butyl (2-fluoro-3-((2-nitrophenyl)sulfonamido)propyl)carbamate (1.94 g, 5.14 mmol) in dry DMF (20 mL) set stirring under nitrogen was added potassium carbonate (2.06 g, 14.92 mmol) and 1,4-diiodobutane (0.32 mL, 2.45 mmol). The solution was heated to 40° C. and allowed to proceed overnight. The following morning, benzyl bromide (0.24 mL, 2.03 mmol) was added, and the reaction was allowed to proceed at room temperature for 8 h. Then, thiophenol (0.96 mL, 9.42 mmol), potassium carbonate (1.01 g, 7.34 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to give an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-80% (75:20:5 DCM/MeOH/concentrated aqueous ammonium hydroxide) gradient. Fractions containing product were combined and concentrated to give di-tert-butyl ((butane-1,4-diylbis(azanediyl))bis(2-fluoropropane-3,1-diyl))dicarbamate as a colorless oil (0.77 g, 1.76 mmol, 72.0%). UPLC/ELSD: RT=0.34 min. MS (ES): m/z (MH⁺) 439.6 for C₂₀H₄₀F₂N₄O₄. ¹H NMR (300 MHz, CDCl₃) δ: ppm 5.11 (m, 2H), 4.73 (br. s, 1H), 4.56 (br. s, 1H), 3.36 (m, 3H), 2.75 (br. m, 9H), 1.51 (m, 5H), 1.43 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate

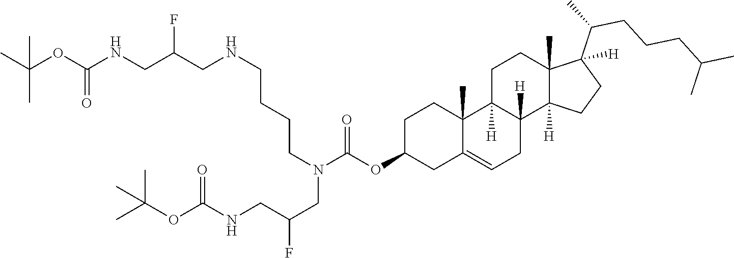

To a solution of di-tert-butyl ((butane-1,4-diylbis(azanediyl))bis(2-fluoropropane-3,1-diyl))dicarbamate (0.73 g, 1.67 mmol) in dry toluene (20 mL) set stirring under nitrogen was added triethylamine (0.59 mL, 4.18 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.77 g, 1.39 mmol) was added, and the solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to give an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate as a colorless oil (0.76 g, 0.89 mmol, 64.1%). UPLC/ELSD: RT=2.62 min. MS (ES): m/z (MH⁺) 852.3 for C₄₈H₈₄F₂N₄O₆. ¹H NMR (300 MHz, CDCl₃) δ: ppm 5.89 (m, 1H), 4.98 (br. m, 1H), 4.76 (br. m, 1H), 4.60 (m, 2H), 3.42 (br. m, 9H), 2.86 (d, 1H, J=6 Hz), 2.78 (m, 1H), 2.65 (m, 3H), 2.35 (m, 2H), 1.90 (br. m, 6H), 1.58 (br. m, 10H), 1.46 (s, 25H), 1.35 (m, 4H), 1.15 (br. m, 10H), 1.04 (s, 6H), 0.94 (d, 4H, J=6 Hz), 0.78 (d, 6H, J=6 Hz), 0.70 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride

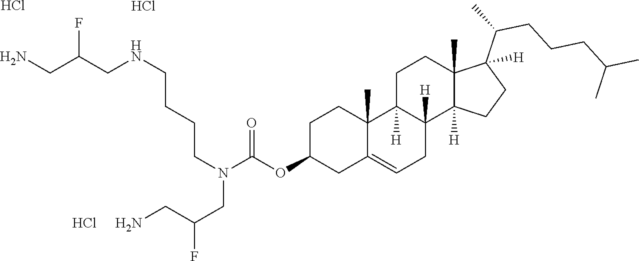

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-fluoropropyl)(4-((3-((tert-butoxycarbonyl)amino)-2-fluoropropyl)amino)butyl)carbamate (0.76 g, 0.89 mmol) in isopropanol (10 mL) set stirring under nitrogen was added hydrochloric acid (5N in isopropanol, 1.79 mL, 8.93 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-fluoropropyl)(4-((3-amino-2-fluoropropyl)amino)butyl)carbamate trihydrochloride as a white solid (0.54 g, 0.67 mmol, 75.4%). UPLC/ELSD: RT=1.65 min. MS (ES): m/z (MH⁺) 651.7 for C₃₈H₇₁C₁₃F₂N₄O₂. ¹H NMR (300 MHz, MeOD) δ: ppm 5.41 (m, 1H), 5.22 (br. m, 1H), 4.48 (br. m, 1H), 3.43 (br. m, 7H), 3.34 (s, 4H), 3.17 (m, 4H), 2.41 (d, 2H, J=3 Hz), 2.05 (br. m, 6H), 1.75 (br. m, 17H), 1.16 (br. m, 13H), 0.95 (d, 4H, J=6 Hz), 0.91 (d, 6H, J=6 Hz), 0.74 (s, 3H).

### AF. Compound SA88: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-hydroxypropyl)(4-((3-amino-2-hydroxypropyl)amino)butyl)carbamate trihydrochloride

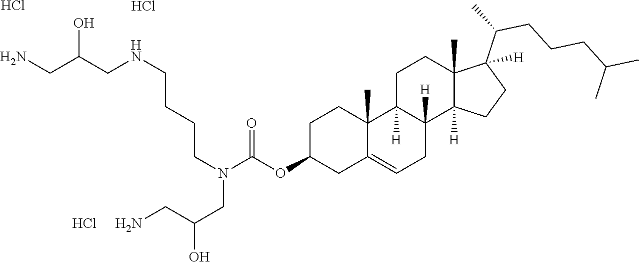

### Step 1: tert-butyl (2-hydroxy-3-((2-nitrophenyl)sulfonamido)propyl)carbamate

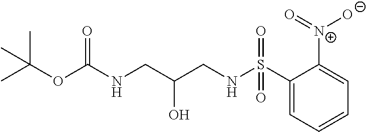 [SMILES: CC(C)(C)OC(=O)NCC(O)CNS(=O)(=O)c1ccccc1[N+](=O)[O-]]

To a solution of tert-butyl (3-amino-2-hydroxypropyl)carbamate (10.51 g, 55.27 mmol) in dry DCM (200 mL) set stirring under nitrogen was added triethylamine (9.24 mL, 66.37 mmol). The solution was cooled to 0° C., and then a solution of 2-nitrobenzenesulfonyl chloride (12.25 g, 55.27 mmol) in 100 mL dry DCM was added dropwise over 30 minutes. The reaction was allowed to proceed at 0° C. for an hour and then at room temperature for an additional three hours. The mixture was then diluted with an additional 10 mL DCM, washed with 1M aqueous sodium bicarbonate (2×100 mL), water (1×100 mL), 10% aqueous citric acid (2×100 mL), water (1×100 mL), and brine (2×100 mL), dried over sodium sulfate, filtered, and concentrated to give tert-butyl (2-hydroxy-3-((2-nitrophenyl)sulfonamido)propyl)carbamate as a white solid (17.54 g, 46.73 mmol, 84.5%). UPLC/ELSD: RT=1.23 min. MS (ES): m/z (MH⁺) 376.4 for C₁₄H₂₁N307S. ¹H NMR (300 MHz, CDCl₃) δ: ppm 8.13 (m, 1H), 7.88 (m, 1H), 7.78 (m, 2H), 6.01 (br. s, 1H), 5.01 (m, 1H), 3.86 (m, 1H), 3.29 (m, 4H), 3.12 (m, 1H), 1.45 (s, 9H).

### Step 2: di-tert-butyl ((butane-1,4-diylbis(azanediyl))bis(2-hydroxypropane-3,1-diyl)dicarbamate

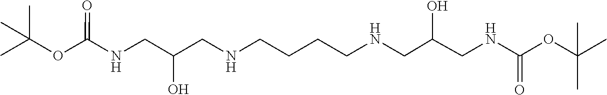 [SMILES: CC(C)(C)OC(=O)NCC(O)CNCCCCNCC(O)CNC(=O)OC(C)(C)C]

To a solution of give tert-butyl (2-hydroxy-3-((2-nitrophenyl)sulfonamido)propyl)carbamate (4.00 g, 10.66 mmol) in dry DMF (40 mL) set stirring under nitrogen was added potassium carbonate (4.28 g, 30.95 mmol) and 1,4-diiodobutane (0.67 mL, 5.07 mmol). The solution was heated to 40° C. and allowed to proceed overnight. The following morning, benzyl bromide (0.50 mL, 4.21 mmol) was added, and the reaction was allowed to proceed at room temperature for 16 h. Then, thiophenol (2.00 mL, 19.54 mmol), potassium carbonate (2.10 g, 15.22 mmol), and an additional 5 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to give an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl ((butane-1,4-diylbis(azanediyl))bis(2-hydroxypropane-3,1-diyl))dicarbamate as a colorless oil (1.12 g, 2.58 mmol, 50.8%). UPLC/ELSD: RT=0.20 min. MS (ES): m/z (MH⁺) 435.6 for C₂₀H₄₂N₄O₆. ¹H NMR (300 MHz, CDCl₃) δ: ppm 5.47 (m, 2H), 4.72 (br. s, 1H), 3.76 (br. s, 2H), 3.47 (m, 9H), 3.22 (m, 3H), 3.05 (m, 3H), 2.62 (br. m, 8H), 1.53 (m, 5H), 1.42 (s, 18H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate

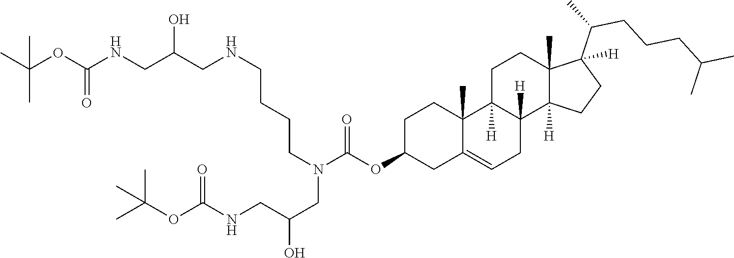

To a solution of di-tert-butyl ((butane-1,4-diylbis(azanediyl))bis(2-hydroxypropane-3,1-diyl))dicarbamate (1.12 g, 2.58 mmol) in dry toluene (20 mL) set stirring under nitrogen was added triethylamine (0.91 mL, 6.44 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.19 g, 2.15 mmol) was added and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (80:19:1 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate as a colorless oil (0.08 g, 0.09 mmol, 4.1%). UPLC/ELSD: RT=2.45 min. MS (ES): m/z (MH⁺) 848.3 for C₄₈H₈₆N₄O₈. ¹H NMR (300 MHz, CDCl₃) δ: ppm 5.32 (m, 2H), 5.07 (br. m, 1H), 4.43 (br. m, 1H), 3.77 (m, 2H), 3.25 (br. m, 6H), 2.95 (m, 4H), 2.56 (m, 4H), 2.26 (m, 2H), 1.90 (m, 5H), 1.47 (m, 9H), 1.37 (s, 18H), 1.07 (m, 11H), 0.95 (s, 6H), 0.86 (d, 4H, J=6 Hz), 0.78 (d, 5H, J=6 Hz), 0.61 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-hydroxypropyl)(4-((3-amino-2-hydroxypropyl)amino)butyl)carbamate trihydrochloride

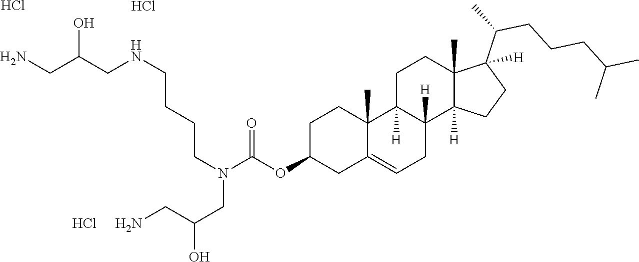

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-2-hydroxypropyl)(4-((3-((tert-butoxycarbonyl)amino)-2-hydroxypropyl)amino)butyl)carbamate (0.08 g, 0.09 mmol) in isopropanol (5 mL) set stirring under nitrogen was added hydrochloric acid (5N in isopropanol, 0.18 mL, 0.89 mmol) dropwise. The solution was heated to 40° C. and allowed to proceed overnight. The following morning, dry acetonitrile (6 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-2-hydroxypropyl)(4-((3-amino-2-hydroxypropyl)amino)butyl)carbamate trihydrochloride as a white solid (0.04 g, 0.04 mmol, 49.3%). UPLC/ELSD: RT=1.53 min. MS (ES): m/z (MH⁺) 648.2 for C₃₈H₇₃Cl₃N₄O₄. ¹H NMR (300 MHz, MeOD) δ: ppm 5.42 (m, 1H), 4.45 (br. m, 1H), 4.26 (br. m, 1H), 4.08 (br. m, 1H), 3.44 (m, 3H), 3.13 (m, 9H), 2.41 (d, 2H, J=3 Hz), 2.05 (s, 3H), 1.92 (m, 3H), 1.55 (br. m, 13H), 1.16 (br. m, 11H), 0.97 (d, 3H, J=6 Hz), 0.91 (d, 5H, J=6 Hz), 0.74 (s, 3H).

### AG. Compound SA89: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

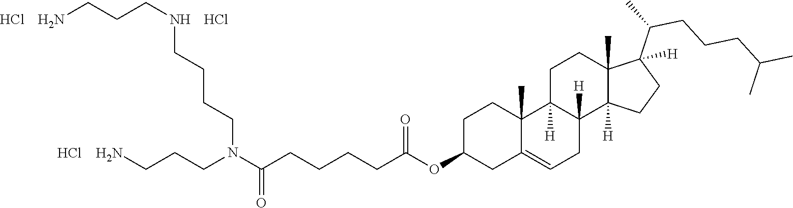

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate

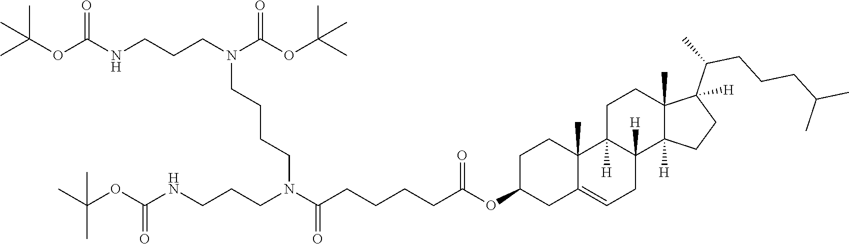

To a solution of 6-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-6-oxohexanoic acid (0.40 g, 0.77 mmol) in dry DCM (10 mL) stirring under nitrogen was added tert-butyl (3-((tert-butoxycarbonyl)amino)propyl)(4-((3-((tert-butoxycarbonyl)amino)propyl)amino)butyl)carbamate (0.43 g, 0.85 mmol), dimethylaminopyridine (0.02 g, 0.15 mmol), and 1-ethyl-3-(3-dimethylaminopropyl)carbodiimide hydrochloride (0.22 g, 1.15 mmol). The resulting solution was cooled to 0° C., and diisopropylethylamine (0.41 mL, 2.31 mmol) was added dropwise. The mixture was allowed to gradually warm to room temperature and proceed overnight. Then, the solution was diluted with dichloromethane, washed with saturated sodium bicarbonate (1×10 mL) and brine (1×10 mL), dried over sodium sulfate, filtered, and concentrated to an oil. The oil was taken up in DCM and purified on silica with a 0-80% ethyl acetate gradient in hexanes. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate as a light yellow oil (0.59 g, 0.59 mmol, 77.0%). UPLC/ELSD: RT: 3.40 min. MS (ES): m/z (MH⁺) 1000.4 for C₅₈H₁₀₂N₄O₉. ¹H NMR (300 MHz, CDCl₃) δ: ppm 5.25 (m, 1H). 4.47 (br. m, 1H), 4.00, (q, 1H), 3.12 (br. m, 12H), 2.20 (br. m, 6H), 1.91 (br. m, 8H), 1.54 (br. m, 16H), 1.31 (br. s, 33H), 1.13 (br. m, 13H), 0.90 (s, 6H), 0.81 (d, 4H, J=6 Hz), 0.73 (d, 6H, J=6 Hz), 0.56 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

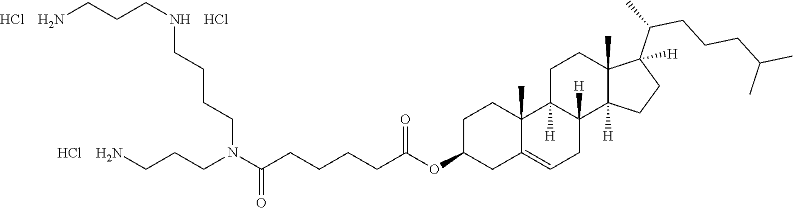

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate (0.59 g, 0.59 mmol) in isopropanol (10 mL) set stirring under nitrogen was added hydrochloric acid (5N in isopropanol, 1.19 mL, 5.92 mmol) dropwise. The solution was heated to 40° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride as a white solid (0.36 g, 0.43 mmol, 71.7%). UPLC/ELSD: RT=1.75 min. MS (ES): m/z (MH⁺) 700.1 for C₄₃H₈₁C₃N₄O₃. ¹H NMR (300 MHz, MeOD) δ: ppm 5.40 (m, 1H), 4.54 (br. m, 1H), 3.96 (m, 1H), 3.52 (br. m, 4H), 3.33 (s, 1H), 3.12 (m, 9H), 2.49 (br. m, 2H), 2.36 (br. m, 5H), 2.17 (m, 3H), 2.06 (s, 3H), 1.67 (br. m, 30H), 1.16 (d, 14H, J=6 Hz), 1.07 (s, 6H), 0.98 (d, 5H, J=6 Hz), 0.89 (d, 7H, J=6 Hz), 0.74 (s, 3H).

### AH. Compound SA90 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

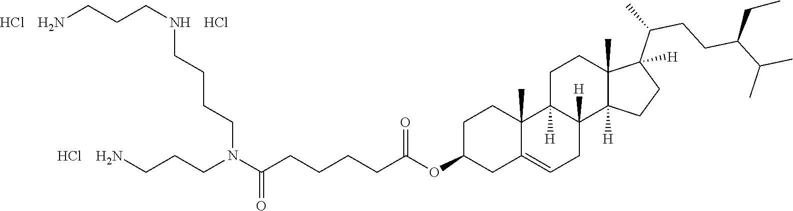

### Step 1: 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid

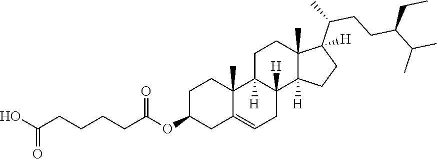

To a solution of sitosterol (0.44 g, 1.01 mmol) in dry DCM (10 mL) stirring under nitrogen was added oxepane-2,7-dione (0.13 g, 1.01 mmol), followed by dropwise addition of pyridine (0.31 mL, 2.22 mmol). The solution was then refluxed at 40° C. overnight, during which all solid went into solution. The following day, the mixture was cooled to room temperature, concentrated to a yellow oil, taken up in DCM, and purified on silica without further workup. The silica column was run in hexanes with a gradient of 0-30% EtOAc. Product-containing fractions were pooled and concentrated to give 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid as a white solid (0.12 g, 0.21 mmol, 21.0%). UPLC/ELSD: RT: 3.23 min. MS (ES): m/z (MH⁺) 543.8 for C₃₅H₅₈O₄. ¹H NMR (300 MHz, CDCl₃) δ: ppm 5.36 (m, 1H), 3.54 (br. m, 1H), 2.28, (m, 2H), 2.04 (br. m, 3H), 1.86 (br. m, 3H), 1.49 (br. m, 19H), 1.02 (s, 6H), 0.94 (d, 5H, J=6 Hz), 0.86 (q, 10H), 0.69 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate

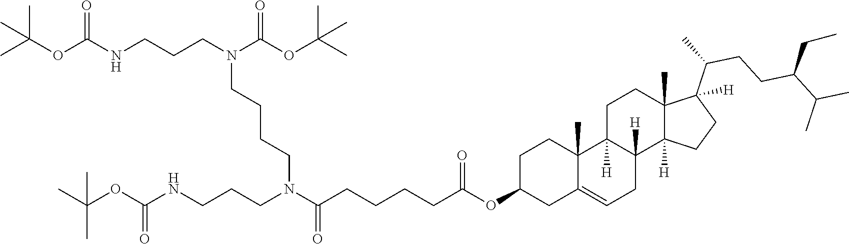

To a solution of 6-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-6-oxohexanoic acid (0.12 g, 0.21 mmol) in dry DCM (10 mL) stirring under nitrogen was added tert-butyl (3-((tert-butoxycarbonyl)amino)propyl)(4-((3-((tert-butoxycarbonyl)amino)propyl)amino)butyl)carbamate (0.13 g, 0.25 mmol), dimethylaminopyridine (0.01 g, 0.04 mmol), and 1-ethyl-3-(3-dimethylaminopropyl)carbodiimide hydrochloride (0.08 g, 0.42 mmol). The resulting solution was cooled to 0° C. and diisopropylethylamine (0.11 mL, 0.64 mmol) was added dropwise. The mixture was allowed to gradually warm to room temperature and proceed overnight. Then, the solution was diluted with dichloromethane, washed with saturated aqueous sodium bicarbonate (1×10 mL) and brine (1×10 mL), dried over sodium sulfate, filtered, and concentrated to give an oil. The oil was taken up in DCM and purified on silica with a 0-80% ethyl acetate gradient in hexanes to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate as a light yellow oil (0.15 g, 0.14 mmol, 67.5%). UPLC/ELSD: RT: 3.91 min. MS (ES): m/z (MH⁺) 1028.5 for C₆₀H₁₀₆N₄O₉. ¹H NMR (300 MHz, CDCl₃) δ: ppm 5.38 (m, 2H), 4.59 (br. m, 2H), 4.14 (m, 1H), 3.24 (br. m, 11H), 2.32 (br. m, 6H), 1.67 (br. m, 17H), 1.47 (s, 32H), 1.25 (br. m, 11H), 1.03 (s, 6H), 0.94 (d, 4H, J=9 Hz), 0.86 (q, 8H, J=9 Hz), 0.69 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride

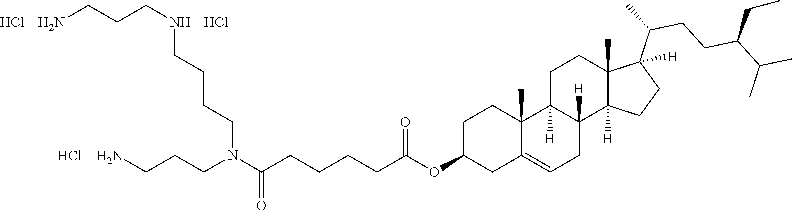

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazaicosan-20-oate (0.15 g, 0.14 mmol) in isopropanol (5 mL) set stirring under nitrogen was added hydrochloric acid (5N in isopropanol, 0.29 mL, 1.43 mmol) dropwise. The solution was heated to 40° C. and allowed to proceed overnight. The following morning, dry acetonitrile (10 mL) was added to the mixture, which was sonicated and allowed to stir for an additional hour. The white solid was then filtered out of the solution, washed repeatedly with acetonitrile, and dried in vacuo to give ((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 6-((3-aminopropyl)(4-((3-aminopropyl)amino)butyl)amino)-6-oxohexanoate trihydrochloride as a white solid (0.06 g, 0.06 mmol, 43.9%). UPLC/ELSD: RT=1.97 min. MS (ES): m/z (MH⁺) 728.1 for C₄₅H₈₅C₃N₄O₃. ¹H NMR (300 MHz, MeOD) δ: ppm 5.41 (m, 1H), 4.87 (br. m, 9H), 4.55 (br. m, 1H), 3.46 (m, 3H), 3.33 (s, 1H), 3.10 (m, 6H), 2.35 (br. m, 6H), 2.04 (s, 5H), 1.68 (br. m, 15H), 1.21 (m, 9H), 1.06 (s, 4H), 0.97 (d, 4H, J=6 Hz), 0.86 (q, 7H, J=6 Hz), 0.74 (s, 3H).

### AI. Compound SA95: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

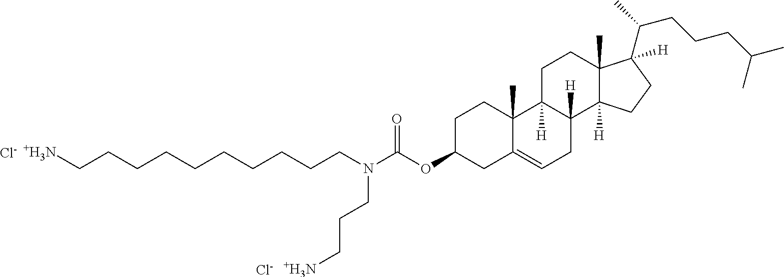

### Step 1: tert-Butyl N-{10-[(2-cyanoethyl)amino]decyl}carbamate

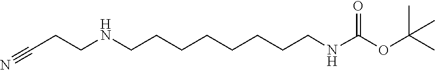 [SMILES: CC(C)(C)OC(=O)NCCCCCCCCNCCC#N]

To a suspension of tert-butyl N-(10-aminodecyl)carbamate (1.500 g, 5.506 mmol), water (15 mL), and glyme (15 mL) was added triton B (cat.). The suspension was stirred at 50° C., and then acrylonitrile (0.40 mL, 6.1 mmol) was added. The reaction mixture stirred at 50° C. and was monitored by LCMS. At 16 h, the reaction mixture was cooled to rt. The reaction mixture was concentrated and diluted with dichloromethane (100 mL) and 5% aq. NaHCO₃ solution (100 mL). The aqueous layer was extracted with dichloromethane (3×30 mL). The combined organics were passed through a hydrophobic frit, dried over Na₂SO₄, and concentrated. The crude material was purified via silica gel chromatography (0-10% methanol in dichloromethane) to afford tert-butyl N-{10-[(2-cyanoethyl)amino]decyl}carbamate (1.328 g, 3.150 mmol, 57.2%) as a white solid. UPLC/ELSD: RT=0.53 min. MS (ES): m/z=326.48 [M+H]⁺ for C₁₈H₃₅N₃O₂; ¹H NMR (300 MHz, CDCl₃): δ 4.49 (br. s, 1H), 3.09 (dt, 2H, J=6.5, 6.3 Hz), 2.93 (t, 2H, J=6.6 Hz), 2.62 (t, 2H, J=7.1 Hz), 2.52 (t, 2H, J=6.6 Hz), 1.22-1.54 (m, 17H), 1.44 (s, 9H).

### Step 2: tert-Butyl N-{10-[benzyl(2-cyanoethyl)amino]decyl}carbamate

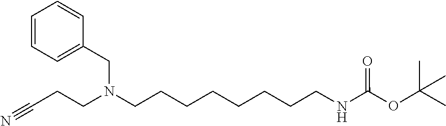 [SMILES: CC(C)(C)OC(=O)NCCCCCCCCN(CCC#N)Cc1ccccc1]

To a stirred suspension of tert-butyl N-{10-[(2-cyanoethyl)amino]decyl}carbamate (1.209 g, 2.867 mmol) and potassium carbonate (0.793 g, 5.74 mmol) in acetonitrile (18 mL) was added benzyl bromide (0.43 mL, 3.6 mmol). The reaction mixture stirred at 70° C. and was monitored by LCMS. At 16 h, the reaction mixture was cooled to rt and then filtered through a pad of Celite rinsing with acetonitrile. The filtrate was concentrated and then purified via silica gel chromatography (0-50% ethyl acetate in hexanes) to afford tert-butyl N-{10-[benzyl(2-cyanoethyl)amino]decyl}carbamate (1.283 g, quant.) as a clear oil. UPLC/ELSD: RT=0.74 min. MS (ES): m/z=416.47 [M+H]⁺ for C₂₅H₄₁N₃O₂; ¹H NMR (300 MHz, CDCl₃): δ 7.20-7.42 (m, 5H), 4.48 (br. s, 1H), 3.61 (s, 2H), 3.10 (td, 2H, J=6.6, 6.3 Hz), 2.78 (t, 2H, J=7.0 Hz), 2.48 (t, 2H, J=7.2 Hz), 2.39 (t, 2H, J=7.0 Hz), 1.39-1.58 (m, 4H), 1.44 (s, 9H), 1.21-1.35 (m, 12H).

### Step 3: Tert-butyl N-{3-[benzyl({10-[(tert-butoxycarbonyl)amino]decyl})amino]propyl}carbamate

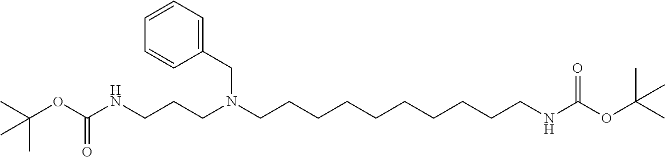 [SMILES: CC(C)(C)OC(=O)NCCCCCCCCCCN(CCCNC(=O)OC(C)(C)C)Cc1ccccc1]

Tert-butyl N-{10-[benzyl(2-cyanoethyl)amino]decyl}carbamate (0.050 g, 0.12 mmol), di-tert-butyl dicarbonate (0.053 g, 0.24 mmol), and nickel (II) chloride (0.016 g, 0.12 mmol) were combined in ethanol (1.0 mL). The stirred reaction mixture was cooled in an ice bath to 0° C., and then sodium borohydride (0.014 g, 0.36 mmol) was added.

The reaction mixture was allowed to come to rt and was monitored by LCMS. At 21 h, diethylenetriamine (0.03 mL, 0.3 mmol) was added. The reaction mixture stirred at rt for 2 h and then filtered through a pad of Celite. The filtrate was concentrated, suspended in a 5% aq. NaHCO₃ solution (25 mL), and extracted with ethyl acetate (3×15 mL). The combined organics were washed with brine, dried over Na₂SO₄, and concentrated. The crude material was purified via silica gel chromatography (0-7% methanol in dichloromethane) to afford tert-butyl N-{3-[benzyl({10-[(tert-butoxycarbonyl)amino]decyl})amino]propyl}carbamate (0.040 g, 0.077 mmol, 64.0%) as a clear oil. UPLC/ELSD: RT=1.25 min. MS (ES): m/z=520.77 [M+H]⁺ for C₃₀H₅₃N₃O₄; ¹H NMR (300 MHz, CDCl₃): δ 7.19-7.39 (m, 5H), 5.52 (br. s, 1H), 4.49 (br. s, 1H), 3.51 (s, 2H), 3.01-3.22 (m, 4H), 2.46 (t, 2H, J=6.1 Hz), 2.37 (t, 2H, J=7.1 Hz), 1.37-1.68 (m, 6H), 1.44 (s, 9H), 1.44 (s, 9H), 1.15-1.35 (m, 12H).

### Step 4: Tert-butyl N-[3-({10-[(tert-butoxycarbonyl)amino]decyl}amino)propyl]carbamate

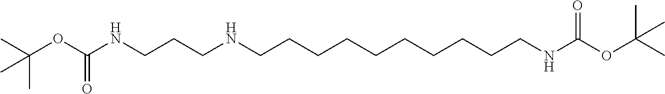 [SMILES: CC(C)(C)OC(=O)NCCCCCCCCCCNCCCNC(=O)OC(C)(C)C]

Tert-butyl N-{3-[benzyl({10-[(tert-butoxycarbonyl)amino]decyl})amino]propyl}carbamate (0.340 g, 0.654 mmol) and 10% Pd/C (0.139 g, 0.065 mmol) were combined in ethanol (5.1 mL) and then stirred under a balloon of H₂ at rt. The reaction was monitored by LCMS. At 18 h the reaction mixture was diluted with ethyl acetate (10 mL) and then was filtered through a pad of Celite rinsing with ethyl acetate. The filtrate was concentrated, taken up in ethyl acetate, filtered through a 0.45 μm PTFE syringe filter, and concentrated to afford tert-butyl N-[3-({10-[(tert-butoxycarbonyl)amino]decyl}amino)propyl]carbamate (0.238 g, 0.554 mmol, 84.7%) as a white solid. UPLC/ELSD: RT=0.97 min. MS (ES): m/z=430.42 [M+H]⁺ for C₂₃H₄₇N₃O₄; ¹H NMR (300 MHz, CDCl₃): δ 5.17 (br. s, 1H), 4.49 (br. s, 1H), 3.20 (dt, 2H, J=6.1, 6.0 Hz), 3.09 (dt, 2H, J=6.6, 6.4 Hz), 2.67 (t, 2H, J=6.6 Hz), 2.58 (t, 2H, J=7.1 Hz), 1.17-1.76 (br. m, 19H), 1.44 (s, 18H).

### Step 5: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate

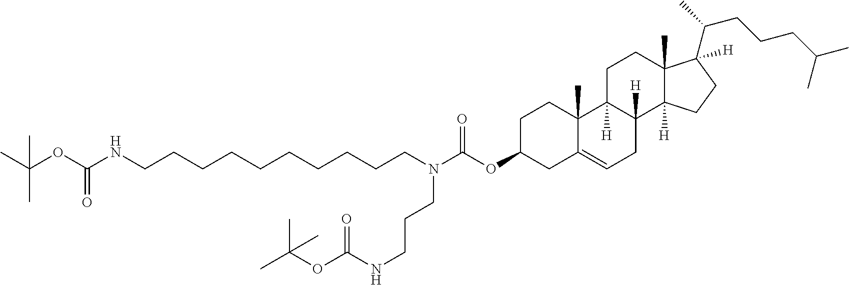

Cholesterol 4-nitrophenyl carbonate (0.112 g, 0.203 mmol), tert-butyl N-[3-({10-[(tert-butoxycarbonyl)amino]decyl}amino)propyl]carbamate (0.103 g, 0.240 mmol), and triethylamine (0.088 mL, 0.63 mmol) were combined in toluene (3.0 mL). The reaction mixture stirred at 90° C. and was monitored by LCMS. At 17 h the reaction mixture was heated at 100° C. At 20 h DMAP (cat.) was added. At 41 h the reaction mixture was cooled to rt, diluted with dichloromethane (20 mL), and then washed with 5% aq. NaHCO₃ solution (3×). The organics were passed through a hydrophobic frit, dried over Na₂SO₄, and concentrated. The crude material was purified via silica gel chromatography (10-30% ethyl acetate in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.157 g, 0.186 mmol, 91.9%) as a clear oil. UPLC/ELSD: RT=3.72 min. MS (ES): m/z=743.62 [(M+H)—(CH₃)₂C═CH₂—CO₂]⁺ for C₅₁H₉₁N₃O₆; ¹H NMR (300 MHz, CDCl₃): δ 5.20-5.43 (m, 2H), 4.40-4.83 (m, 2H), 3.02-3.37 (m, 8H), 2.21-2.43 (m, 2H), 1.75-2.10 (m, 5H), 0.93-1.75 (br. m, 39H), 1.44 (s, 9H), 1.44 (s, 9H), 1.02 (s, 3H), 0.92 (d, 3H, J=6.4 Hz), 0.87 (d, 3H, J=6.5 Hz), 0.86 (d, 3H, J=6.5 Hz), 0.68 (s, 3H).

### Step 6: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

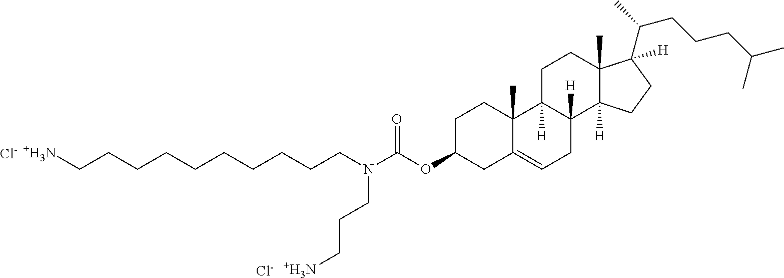

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.154 g, 0.183 mmol) in isopropanol (2.5 mL) was added 5-6 N HCl in isopropanol (0.28 mL). The reaction mixture stirred at 40° C. and was monitored by LCMS. At 17 h the reaction mixture was cooled to rt and then acetonitrile (7.5 mL) was added. Solids were collected by vacuum filtration rinsing with cold 3:1 acetonitrile/isopropanol to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride (0.080 g, 0.11 mmol, 59.2%) as a white solid. UPLC/ELSD: RT=2.23 min. MS (ES): m/z=342.41 [(M+2H)+CH₃CN]²⁺ for C₄₁H₇₅N₃O₂; ¹H NMR (300 MHz, CD₃OD): δ 5.35-5.45 (m, 1H), 4.36-4.51 (m, 1H), 3.36 (t, 2H, J=6.9 Hz), 3.26 (t, 2H, J=7.4 Hz), 2.87-2.98 (m, 4H), 2.27-2.43 (m, 2H), 1.78-2.12 (m, 7H), 0.97-1.73 (br. m, 37H), 1.06 (s, 3H), 0.95 (d, 3H, J=6.4 Hz), 0.88 (d, 3H, J=6.6 Hz), 0.88 (d, 3H, J=6.6 Hz), 0.73 (s, 3H).

### AJ. Compound SA96: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

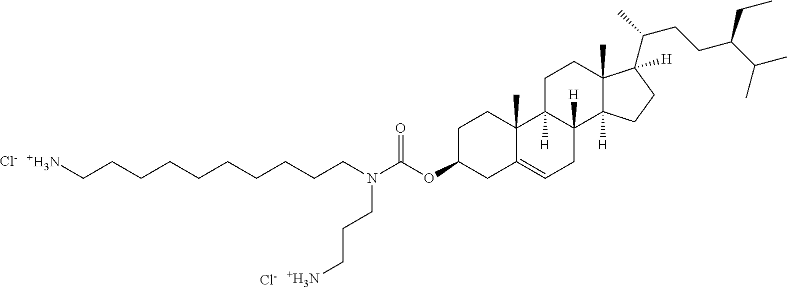

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate

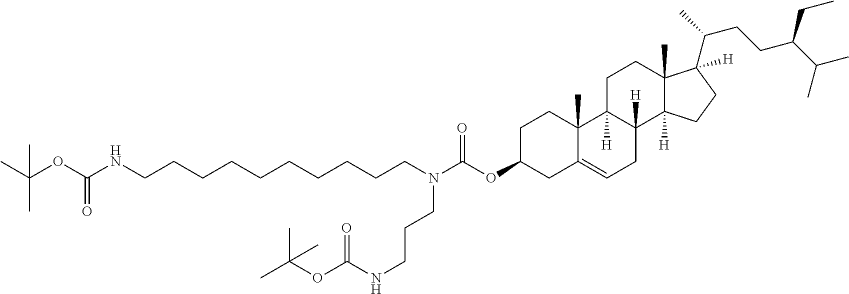

β-Sitosterol 4-nitrophenyl carbonate (0.140 g, 0.241 mmol), tert-butyl N-[3-({10-[(tert-butoxycarbonyl)amino]decyl}amino)propyl]carbamate (0.119 g, 0.277 mmol), and triethylamine (0.10 mL, 0.75 mmol) were combined in toluene (3.5 mL). The reaction mixture stirred at 90° C. and was monitored by LCMS. At 17 h the reaction mixture was heated to 100° C. At 20 h DMAP (cat.) was added. At 41 h the reaction mixture was cooled to rt, diluted with dichloromethane (20 mL), and then washed with 5% aq. NaHCO₃ solution (3×). The organics were passed through a hydrophobic frit, dried over Na₂SO₄, and concentrated. The crude material was purified via silica gel chromatography (10-30% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.187 g, 0.215 mmol, 89.0%) as a clear oil. UPLC/ELSD: RT=3.80 min. MS (ES): m/z=770.03 [(M+H)—(CH₃)₂C═CH₂—CO₂]⁺ for C₅₃H₉₅N₃O₆; ¹H NMR (300 MHz, CDCl₃): δ 5.18-5.43 (m, 2H), 4.40-4.83 (m, 2H), 2.97-3.41 (m, 8H), 2.21-2.44 (m, 2H), 1.76-2.13 (m, 5H), 0.87-1.74 (br. m, 40H), 1.44 (s, 9H), 1.44 (s, 9H), 1.02 (s, 3H), 0.92 (d, 3H, J=6.4 Hz), 0.78-0.87 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride

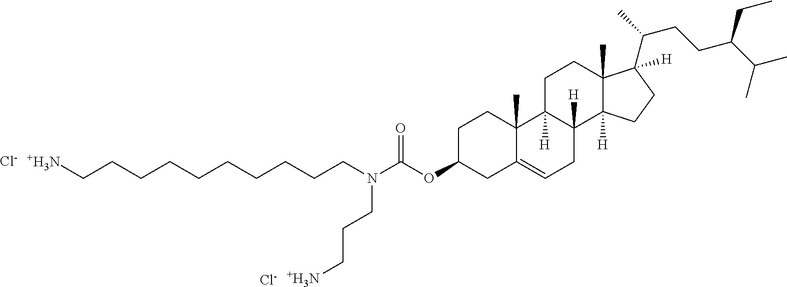

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-((tert-butoxycarbonyl)amino)decyl)(3-((tert-butoxycarbonyl)amino)propyl)carbamate (0.182 g, 0.209 mmol) in isopropanol (2.5 mL) was added 5-6 N HCl in isopropanol (0.28 mL). The reaction mixture stirred at 40° C. and was monitored by LCMS. At 17 h the reaction mixture was cooled to rt and then acetonitrile (8.25 mL) was added. The mixture was concentrated. Methyl tert-butyl ether (ca. 10 mL) was added. The mixture was concentrated. The residue was dissolved in isopropanol (1.5 mL) and then added dropwise to acetonitrile (10 mL). The mixture was concentrated. Acetonitrile/methyl tert-butyl ether/isopropanol (85:10:5, ca. 10 mL) was added. The mixture was concentrated. The residue was dissolved in isopropanol (1.5 mL) and then added dropwise to 3:1 hexanes/ethyl acetate (10 mL). The mixture was concentrated. The residue was dissolved in isopropanol (1.5 mL) and then 9:1 acetonitrile/ethanol (10 mL) was added. Then acetonitrile (10 mL) was added. The supernatant was decanted, and solids were dried under vacuum to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (10-aminodecyl)(3-aminopropyl)carbamate dihydrochloride (0.075 g, 0.089 mmol, 42.7%) as a white solid. UPLC/ELSD: RT=2.34 min. MS (ES): m/z=356.73 [(M+2H)+CH₃CN]²⁺ for C₄₃H₇₉N₃O₂; ¹H NMR (300 MHz, CD₃OD): δ 5.36-5.46 (m, 1H), 4.36-4.52 (m, 1H), 3.36 (t, 2H, J=6.8 Hz), 3.26 (t, 2H, J=7.3 Hz), 2.87-2.98 (m, 4H), 2.27-2.44 (m, 2H), 1.80-2.12 (m, 7H), 0.91-1.77 (br. m, 38H), 1.06 (s, 3H), 0.96 (d, 3H, J=6.4 Hz), 0.79-0.91 (m, 9H), 0.73 (s, 3H).

### AK. Compound SA97: N-(8-Aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

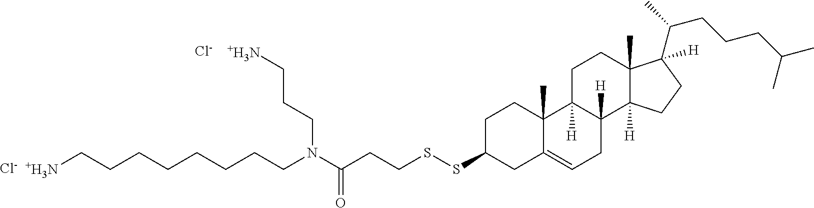

### Step 1: 2-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine

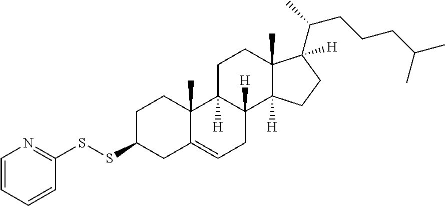

Thiocholesterol (2.000 g, 4.966 mmol) and 2,2′-dipyridyldisulfide (1.204 g, 5.463 mmol) were combined in chloroform (12.5 mL). The reaction mixture stirred at rt and was monitored by LCMS. At 20 h the reaction mixture was concentrated, and then methanol (35 mL) was added. The resulting mixture was let stand for 2 h. After this time, solids were ground with methanol as a slurry by mortar and pestle, and then solids were collected by vacuum filtration to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine (1.812 g, 3.540 mmol, 71.3%) as a light tan solid. UPLC/ELSD: RT=3.45 min. MS (ES): m/z=512.62 [M+H]⁺ for C₃₂H₄₉NS₂; ¹H NMR (300 MHz, CDCl₃): δ 8.39-8.49 (m, 1H), 7.72-7.83 (m, 1H), 7.57-7.69 (m, 1H), 7.01-7.12 (m, 1H), 5.27-5.43 (m, 1H), 2.70-2.88 (m, 1H), 2.20-2.47 (m, 2H), 0.78-2.11 (br. m, 38H), 0.66 (s, 3H).

### Step 2: 2-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate

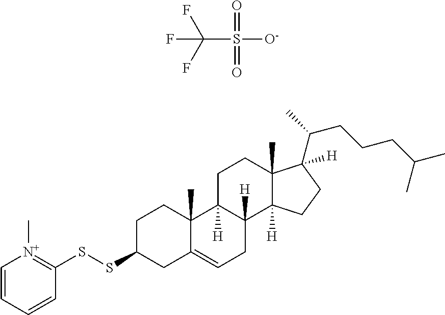

To a solution of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pyridine (1.807 g, 3.530 mmol) in dichloromethane (3.5 mL) and heptanes (35 mL) was added methyl trifluoromethanesulfonate (0.40 mL, 3.5 mmol) dropwise over 10 min. The reaction mixture stirred at rt and was monitored by TLC. At 4 h additional trifluoromethanesulfonate (0.08 mL) was added dropwise. At 4 h 40 min solids were collected via vacuum filtration rinsing with heptanes to afford 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.011 g, 2.975 mmol, 84.3%) as an off-white solid. ¹H NMR (300 MHz, CD₃CN): δ 8.51-8.63 (m, 2H), 8.30-8.41 (m, 1H), 7.66-7.75 (m, 1H), 5.34-5.45 (m, 1H), 4.19 (s, 3H), 2.87-3.06 (m, 1H), 2.33-2.49 (m, 2H), 0.78-2.08 (br. m, 38H), 0.69 (s, 3H).

### Step 3: 3-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid

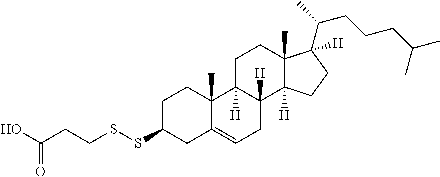

To a mixture of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (1.000 g, 1.479 mmol) in dimethylformamide (6.5 mL) was added 3-mercaptopropionic acid (0.14 mL, 1.6 mmol). The reaction mixture stirred at rt and was monitored by LCMS. At 21 h additional 3-mercaptopropionic acid (0.02 mL) was added. At 24 h water (8 mL) was added, and the reaction mixture stirred at rt for 30 min and then was sonicated. Solids were collected by vacuum filtration rinsing with water. The wet solids were then dissolved in dichloromethane and passed through a hydrophobic frit, dried over Na₂SO₄, and concentrated. Acetonitrile (40 mL) was added to the residue, which was then sonicated. Solids were collected by vacuum filtration to afford 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.541 g, 1.07 mmol, 72.2%) as a white solid.

UPLC/ELSD: RT=3.26 min.; ¹H NMR (300 MHz, CDCl₃): δ 10.32 (br. s, 1H), 5.31-5.40 (m, 1H), 2.86-2.95 (m, 2H), 2.75-2.84 (m, 2H), 2.59-2.73 (m, 1H), 2.23-2.41 (m, 2H), 1.74-2.08 (m, 5H), 0.93-1.70 (br. m, 21H), 1.00 (s, 3H), 0.92 (d, 3H, J=6.5 Hz), 0.87 (d, 3H, J=6.6 Hz), 0.86 (d, 3H, J=6.6 Hz), 0.68 (s, 3H).

### Step 4: tert-Butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate

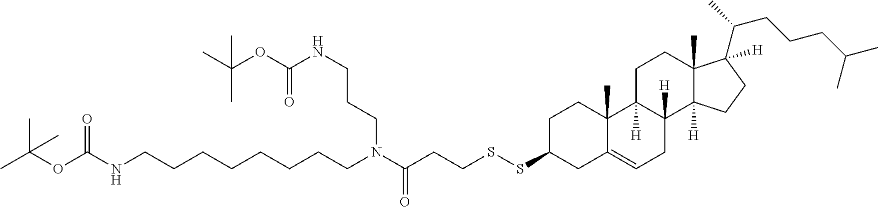

To a mixture of 3-(((3 S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.200 g, 0.395 mmol), tert-butyl N-[3-({8-[(tert-butoxycarbonyl)amino]octyl}amino)propyl]carbamate (0.222 g, 0.552 mmol), and N-hydroxysuccinimide (0.068 g, 0.59 mmol) in dichloromethane (6.0 mL) was added dicyclohexylcarbodiimide (0.138 g, 0.671 mmol). The reaction mixture stirred at rt and was monitored by LCMS. At 17 h, additional N-hydroxysuccinimide (15 mg) and dicyclohexylcarbodiimide (35 mg) were added. At 5 days the reaction mixture was filtered through a pad of Celite rinsing with dichloromethane. The filtrate was concentrated, taken up in 9:1 hexanes/ethyl acetate (10 mL), filtered, and concentrated. The crude material was purified via silica gel chromatography (10-50% ethyl acetate in hexanes) to afford tert-butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate (0.203 g, 0.228 mmol, 57.8%) as a clear oil. UPLC/ELSD: RT=3.61 min. MS (ES): m/z=790.32 [(M+H)—(CH₃)₂C═CH₂—CO₂]⁺ for C₅₁H₉₁N₃O₅S₂; ¹H NMR (300 MHz, CDCl₃): δ 5.24-5.42 (m, 2H), 4.42-4.67 (m, 1H), 2.91-3.48 (br. m, 10H), 2.57-2.78 (m, 3H), 2.27-2.38 (m, 2H), 0.93-2.09 (br. m, 40H), 1.44 (s, 9H), 1.43 (s, 9H), 1.00 (s, 3H), 0.91 (d, 3H, J=6.4 Hz), 0.87 (d, 3H, J=6.5 Hz), 0.86 (d, 3H, J=6.6 Hz), 0.68 (s, 3H).

### Step 5: N-(8-Aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

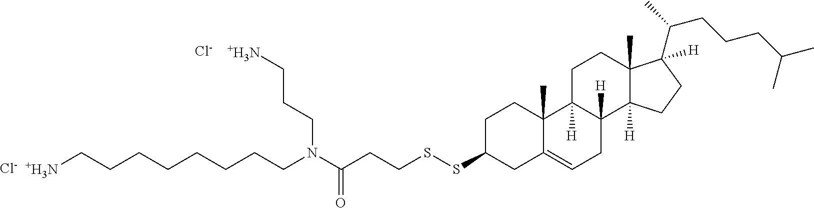

To a mixture of tert-butyl (3-(N-(8-((tert-butoxycarbonyl)amino)octyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)propyl)carbamate (0.200 g, 0.225 mmol) in isopropanol (3.0 mL) was added 5-6 N HCl in isopropanol (0.32 mL). The reaction mixture stirred at 40° C. and was monitored by LCMS. At 17 h the reaction mixture was cooled to rt, and then acetonitrile (9 mL) was added. The material was concentrated and then taken up in 4:1 acetonitrile/methanol (10 mL). The suspension was filtered rinsing with methanol. The filtrate was concentrated, triturated with 19:1 acetonitrile/ethanol (10 mL), dissolved in methanol, and concentrated to afford N-(8-aminooctyl)-N-(3-aminopropyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.126 g, 0.154 mmol, 68.4%) as a white solid. UPLC/ELSD: RT=2.28 min. MS (ES): m/z=366.60 [(M+2H)+CH₃CN]²⁺ for C₄₁H₇₅N₃OS₂.

### AL. Compound SA98 (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride

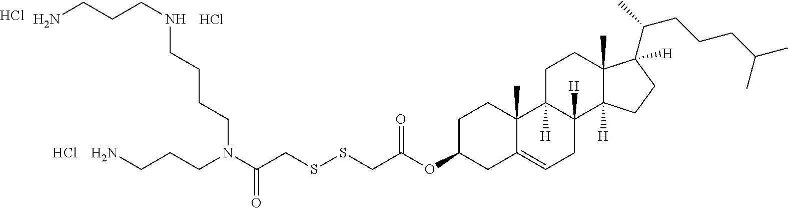

### Step 1: 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid

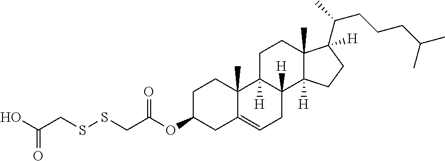

To a solution of cholesterol (5.00 g, 12.93 mmol) in dry DCM (100 mL) stirring under nitrogen was added dithiodiglycolic acid (4.53 mL, 25.86 mmol). The solution was then cooled to 0° C. and dimethylaminopyridine (0.32 g, 2.59 mmol) and 1-ethyl-3-(3-dimethylaminopropyl)carbodiimide hydrochloride (4.96 g, 25.86 mmol) were added, followed by dropwise addition of triethylamine (4.52 mL, 25.86 mmol). The solution was allowed to gradually warm to room temperature and stir overnight. The following day, the solution was washed with saturated sodium bicarbonate (1×25 mL) and water (1×25 mL), dried over sodium sulfate, filtered, and concentrated to a brown oil. The oil was taken up in DCM and purified on silica in hexanes with a 0-100% EtOAc gradient. Product-containing fractions were pooled and concentrated to give 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid as a dark brown solid (3.76 g, 6.82 mmol, 52.7%). UPLC/ELSD: RT: 3.11 min. MS (ES): m/z (MH⁺) 551.8 for C₃₁H₅₀O₄S₂. ¹H NMR (300 MHz, CDCl₃) δ: ppm 9.04 (br. s, 1H), 5.41 (m, 1H), 4.69 (br. m, 1H), 3.65 (s, 2H), 3.60 (s, 1H), 2.39 (d, 2H, J=9 Hz), 2.01 (br. m, 5H), 1.52 (br. m, 11H), 1.16 (br. m, 6H), 1.04 (s, 6H), 0.95 (d, 3H, J=6 Hz), 0.86 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate

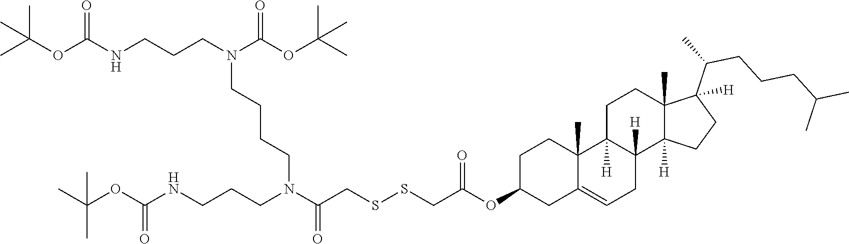

To a solution of 2-((2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-2-oxoethyl)disulfaneyl)acetic acid (0.30 g, 0.55 mmol) in dry DCM (5 mL) stirring under nitrogen was added tert-butyl (3-((tert-butoxycarbonyl)amino)propyl)(4-((3-((tert-butoxycarbonyl)amino)propyl)amino)butyl)carbamate (0.41 g, 0.82 mmol), dimethylaminopyridine (0.03 g, 0.27 mmol), and 1-ethyl-3-(3-dimethylaminopropyl)carbodiimide hydrochloride (0.26 g, 1.36 mmol). The reaction was allowed to proceed at room temperature overnight. Then, the solution was diluted with dichloromethane, washed with saturated aqueous sodium bicarbonate (1×10 mL) and brine (1×10 mL), dried over sodium sulfate, filtered, and concentrated to an oil. The oil was taken up in DCM and purified on silica with a 0-80% ethyl acetate gradient in hexanes to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate as a light yellow oil (0.33 g, 0.31 mmol, 57.6%). UPLC/ELSD: RT: 3.46 min. MS (ES): m/z (MH⁺) 1036.5 for C₅₆H₉₈N₄O₉S₂. ¹H NMR (300 MHz, CDCl₃) δ: ppm 5.33 (m, 2H), 4.61 (br. m, 1H), 3.72 (s, 2H), 3.51 (s, 2H), 3.29 (br. m, 11H), 2.28 (d, 2H, J=6 Hz), 1.81 (br. m, 6H), 1.50 (s, 26H), 1.20 (br. m, 11H), 0.97 (s, 5H), 0.88 (d, 3H, J=6 Hz), 0.82 (d, 5H, J=6 Hz), 0.63 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride

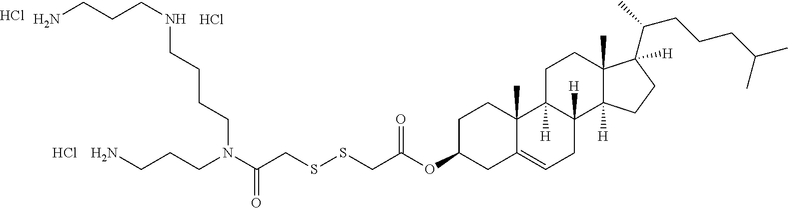

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 12-(tert-butoxycarbonyl)-7-(3-((tert-butoxycarbonyl)amino)propyl)-19,19-dimethyl-6,17-dioxo-18-oxa-3,4-dithia-7,12,16-triazaicosanoate (0.33 g, 0.31 mmol) in isopropanol (5 mL) set stirring under nitrogen was added hydrochloric acid (5N in isopropanol, 0.63 mL, 3.14 mmol) dropwise. The solution was heated to 40° C. and allowed to proceed overnight. The following morning, the solution was cooled to room temperature, and dry acetonitrile (10 mL) was added to the mixture, which was sonicated and allowed to stir for an additional hour. The white solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((2-((3-aminopropyl)(4-((3-aminopropyl)amino)butyl)amino)-2-oxoethyl)disulfaneyl)acetate trihydrochloride as a white solid (0.21 g, 0.22 mmol, 70.0%). UPLC/ELSD: RT=2.00 min. MS (ES): m/z (MH⁺) 735.7 for C₄₁H₇₇Cl₃N₄O₃S₂. ¹H NMR (300 MHz, MeOD) δ: ppm 5.43 (m, 1H), 4.60 (br. m, 1H), 3.90 (m, 2H), 3.67 (s, 2H), 3.53 (m, 5H), 3.33 (s, 2H), 3.11 (m, 9H), 2.39 (m, 2H), 1.98 (br. m, 10H), 1.55 (br. m, 13H), 1.39 (m, 7H), 1.18 (br. m, 6H), 1.08 (s, 6H), 0.98 (d, 4H, J=6 Hz), 0.91 (d, 6H, J=6 Hz), 0.75 (s, 3H).

### AM. Compound SA110: N-(8-Aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride

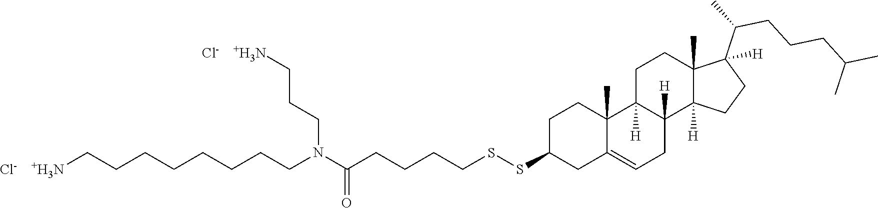

### Step 1: 5-(((3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid

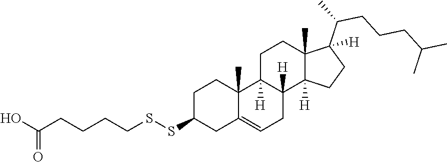

To a stirred mixture of 2-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (1.000 g, 1.479 mmol) in dimethylformamide (4.5 mL) was added 5-sulfanylpentanoic acid (0.208 g, 1.55 mmol) in dimethylformamide (2.0 mL). The reaction mixture stirred at rt and was monitored by LCMS. At 15 h additional 5-sulfanylpentanoic acid (60 mg) in dimethylformamide (0.5 mL) was added. At 40 h water (20 mL) was added, and the reaction mixture stirred at rt for 15 min and then was sonicated. Solids were collected by vacuum filtration and were rinsed with water. The solids were dissolved in dichloromethane, passed through a hydrophobic frit, dried over Na₂SO₄, and concentrated. Acetonitrile (25 mL) was added to the residue, and the suspension was sonicated. Solids were collected by vacuum filtration rinsing sparingly with cold acetonitrile to afford 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid (0.604 g, 1.13 mmol, 76.3%) as a white solid. UPLC/ELSD: RT=3.47 min; ¹H NMR (300 MHz, CDCl₃): δ 10.10 (br. s, 1H), 5.30-5.48 (m, 1H), 2.57-2.77 (m, 3H), 2.22-2.46 (m, 4H), 0.94-2.08 (br. m, 30H), 1.01 (s, 3H), 0.92 (d, 3H, J=6.5 Hz), 0.87 (d, 3H, J=6.6 Hz), 0.86 (d, 3H, J=6.6 Hz), 0.68 (s, 3H).

### Step 2: tert-Butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamido)octyl)carbamate

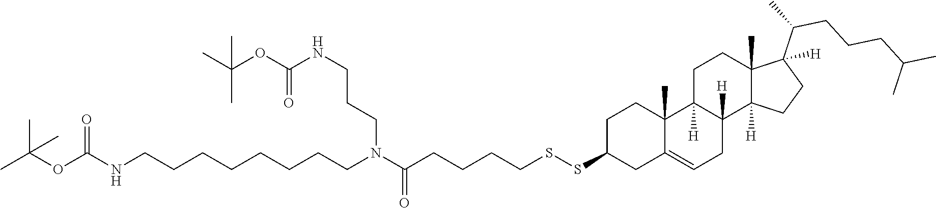

To a mixture of 5-(((3 S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanoic acid (0.250 g, 0.467 mmol), tert-butyl N-[3-({8-[(tert-butoxycarbonyl)amino]octyl}amino)propyl]carbamate (0.263 g, 0.654 mmol), and N-hydroxysuccinimide (0.081 g, 0.70 mmol) in dichloromethane (7.5 mL) was added dicyclohexylcarbodiimide (0.164 g, 0.795 mmol). The reaction mixture stirred at rt and was monitored by LCMS. At 50 h N-hydroxysuccinimide (34 mg) and dicyclohexylcarbodiimide (72 mg) were added. At 92 h hexanes (38 mL) was added, and then the reaction mixture was filtered through a pad of Celite rinsing with 5:1 hexanes/dichloromethane. The filtrate was concentrated and then purified via silica gel chromatography (10-50% ethyl acetate in hexanes) to afford tert-butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamido)octyl)carbamate (0.210 g, 0.229 mmol, 48.9%) as a clear oil. UPLC/ELSD: RT=3.53 min. MS (ES): m/z=919.93 [M+H]⁺ for C₅₃H₉₅N₃O₅S₂; ¹H NMR (300 MHz, CDCl₃): δ 5.23-5.48 (m, 2H), 4.38-4.67 (m, 1H), 2.99-3.45 (br. m, 8H), 2.56-2.76 (m, 3H), 2.22-2.41 (m, 4H), 0.93-2.08 (br. m, 44H), 1.44 (s, 9H), 1.43 (s, 9H), 1.00 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 3: N-(8-Aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride

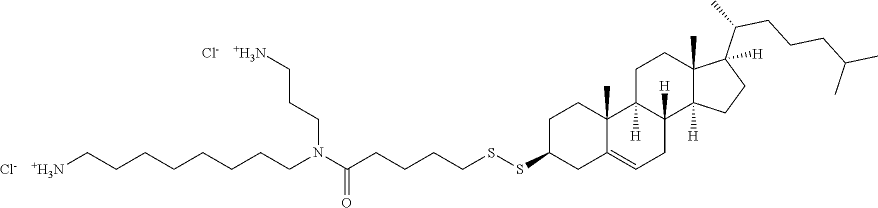

To a mixture of tert-butyl (8-(N-(3-((tert-butoxycarbonyl)amino)propyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamido)octyl)carbamate (0.208 g, 0.226 mmol) in isopropanol (3.0 mL) was added 5-6 N HCl in isopropanol (0.32 mL). The reaction mixture stirred at 40° C. and was monitored by LCMS. At 24 h the reaction mixture was diluted with methanol (3 mL) and filtered rinsing with methanol. The filtrate was concentrated, and then the residue was triturated with 19:1 acetonitrile/ethanol (2×3 mL). The residue was dissolved in methanol and then concentrated to afford N-(8-aminooctyl)-N-(3-aminopropyl)-5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)pentanamide dihydrochloride as a white foam (0.111 g, 0.140 mmol, 62.0%). UPLC/ELSD: RT=2.28 min. MS (ES): m/z=359.81 [M+2H]²⁺ for C₄₃H₇₉N₃OS₂.

¹H NMR (300 MHz, DMSO, reported as seen in spectrum): δ 7.67-8.29 (m, 8.78H), 5.25-5.43 (m, 1H), 3.14-3.43 (m, 7.91H), 2.58-2.86 (m, 10.97H), 2.17-2.39 (m, 5.35H), 0.79-2.07 (br m, 84.51H), 0.61-0.70 (m, 3.34H).

### AN. Compound SA111 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

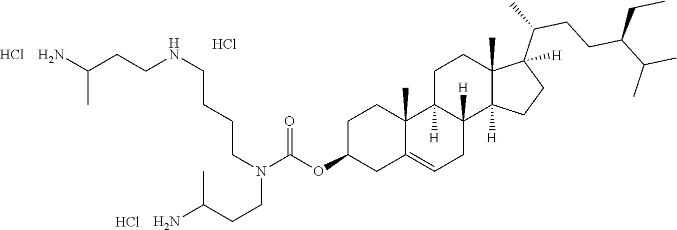

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate

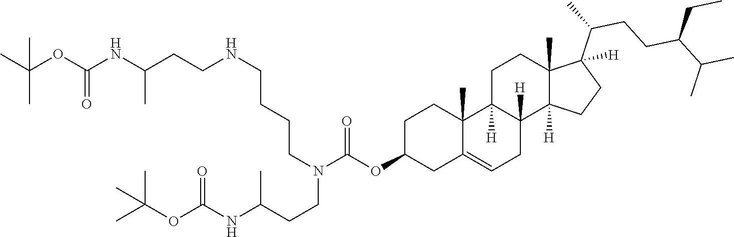

To a solution of di-tert-butyl ((butane-1,4-diylbis(azanediyl))bis(butane-4,2-diyl))dicarbamate (0.33 g, 0.77 mmol) in dry toluene (10 mL) set stirring under nitrogen was added triethylamine (0.32 mL, 2.30 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.44 g, 0.77 mmol) was added and the solution was heated to 90° C. and allowed to proceed overnight. The following morning, the reaction mixture was allowed to cool to room temperature, diluted with toluene, and washed with water (3×20 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate as a light yellow oil (0.41 g, 0.48 mmol, 62.0%). UPLC/ELSD: RT=2.76 min. MS (ES): m/z (MH⁺) 872.3 for C₅₂H₉₄N₄O₆. ¹H NMR (300 MHz, CDCl₃) δ: ppm 5.08 (m, 1H), 4.67 (br. m, 1H), 4.19 (br. m, 2H), 3.42 (m, 2H), 3.13 (s, 3H), 2.90 (br. m, 4H), 2.29 (m, 4H), 2.05 (m, 4H), 1.69 (m, 6H), 1.35 (br. m, 14H), 1.14 (br. s, 17H), 0.99 (br. m, 6H), 0.86 (d, 9H, J=6 Hz), 0.73 (s, 5H), 0.64 (d, 5H, J=6 Hz), 0.55 (q, 8H, J=6 Hz), 0.38 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride

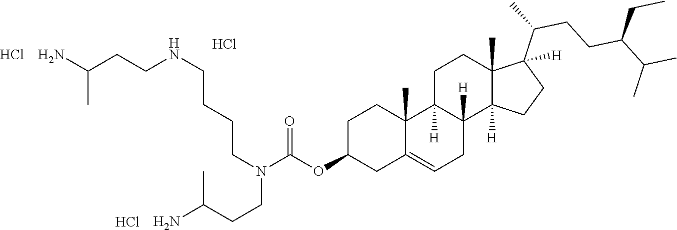

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(4-((3-((tert-butoxycarbonyl)amino)butyl)amino)butyl)carbamate (0.41 g, 0.48 mmol) in isopropanol (7 mL) set stirring under nitrogen was added hydrochloric acid (5N in isopropanol, 0.95 mL, 4.75 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(4-((3-aminobutyl)amino)butyl)carbamate trihydrochloride as a white solid (0.30 g, 0.36 mmol, 74.8%). UPLC/ELSD: RT=1.50 min. MS (ES): m/z (MH⁺) 672.3 for C₄₂H₈₁C₃N₄O₂. ¹H NMR (300 MHz, MeOD) δ: ppm 5.41 (m, 1H), 4.48 (br. m, 1H), 3.48 (br. m, 2H), 3.33 (s, 7H), 3.17 (m, 3H), 2.39 (d, 2H, J=3 Hz), 1.92 (br. m, 8H), 1.73 (br. m, 10H), 1.37 (br. m, 9H), 1.17 (d, 4H, J=6 Hz), 1.07 (s, 5H), 0.98 (d, 5H, J=6 Hz), 0.86 (q, 8H, J=6 Hz), 0.74 (s, 3H).

### AO. Compound SA113: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride

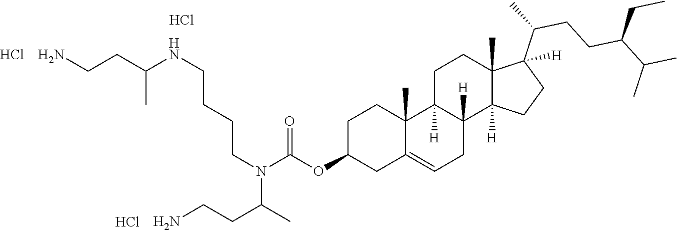

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate

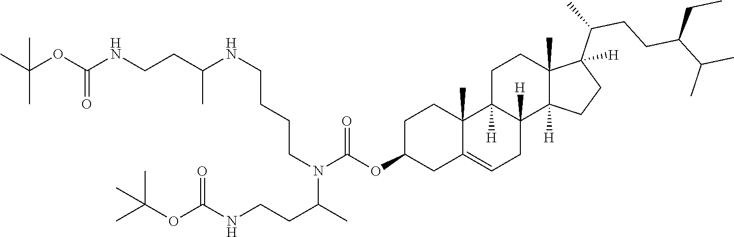

To a solution of di-tert-butyl ((butane-1,4-diylbis(azanediyl))bis(butane-3,1-diyl))dicarbamate (0.19 g, 0.43 mmol) in dry toluene (10 mL) set stirring under nitrogen was added triethylamine (0.18 mL, 1.31 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.25 g, 0.44 mmol) was added. The solution was heated to 90° C. and allowed to proceed for 48 h. Then, the reaction mixture was allowed to cool to room temperature, diluted with toluene, washed with water (3×20 mL), dried over sodium sulfate, filtered, and concentrated to give an oil. The oil was taken up in DCM and purified via silica gel chromatography in DCM with a 0-80% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate as a light yellow oil (0.16 g, 0.18 mmol, 41.0%).

UPLC/ELSD: RT=2.50 min. MS (ES): m/z (MH⁺) 872.3 for C₅₂H₉₄N₄O₆. ¹H NMR (300 MHz, CDCl₃) δ: ppm 5.34 (br. m, 2H), 4.48 (br. m, 1H), 4.17 (br. m, 1H), 3.15 (br. m, 6H), 2.48 (br. m, 7H), 1.95 (br. m, 7H), 1.51 (br. m, 15H), 1.39 (s, 25H), 1.11 (br. m, 15H), 1.04 (d, 5H, J=6 Hz), 0.98 (s, 6H), 0.89 (d, 6H, J=6 Hz), 0.78 (q, 10H, J=6 Hz), 0.64 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-JH-cyclopenta[a]phenanthren-3-yl (4-aminobutan-2-yl)(4-((4-aminobutan-2-yl)amino)butyl)carbamate trihydrochloride

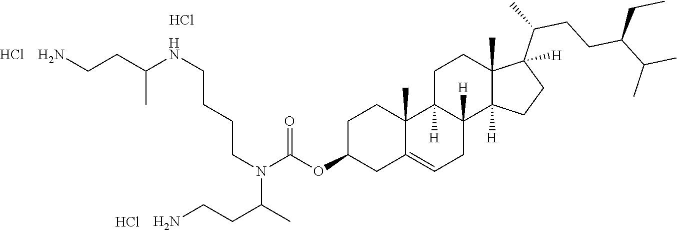

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butan-2-yl)(4-((4-((tert-butoxycarbonyl)amino)butan-2-yl)amino)butyl)carbamate (0.16 g, 0.18 mmol) in isopropanol (5 mL) set stirring under nitrogen was added hydrochloric acid (5N in isopropanol, 0.36 mL, 1.80 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutan-2-yl)(4-((4-aminobutan-2-yl)amino)butyl)carbamate trihydrochloride as a white solid (0.11 g, 0.13 mmol, 72.0%).

UPLC/ELSD: RT=1.69 min. MS (ES): m/z (MH⁺) 673.2 for C₄₂H₈₁C₃N₄O₂. ¹H NMR (300 MHz, MeOD) δ: ppm 5.43 (m, 1H), 4.47 (br. m, 1H), 4.12 (m, 1H), 3.43 (br. m, 1H), 3.33 (s, 4H), 3.24 (br. m, 2H), 3.13 (m, 5H), 2.91 (br. m, 2H), 2.41 (d, 2H, J=3 Hz), 1.99 (br. m, 10H), 1.76 (br. m, 12H), 1.43 (d, 6H, J=6 Hz), 1.31 (d, 6H, J=6 Hz), 1.18 (br. m, 6H), 1.08 (s, 6H), 0.99 (d, 5H, J=6 Hz), 0.89 (q, 9H, J=6 Hz), 0.75 (s, 3H).

### AP. Compound SA114 (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

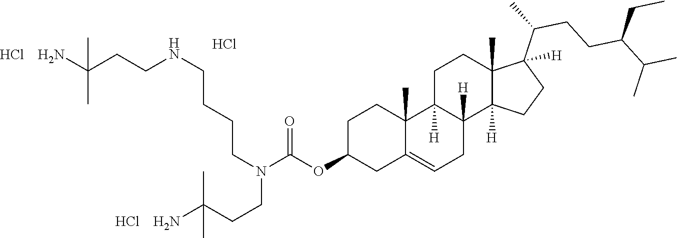

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate

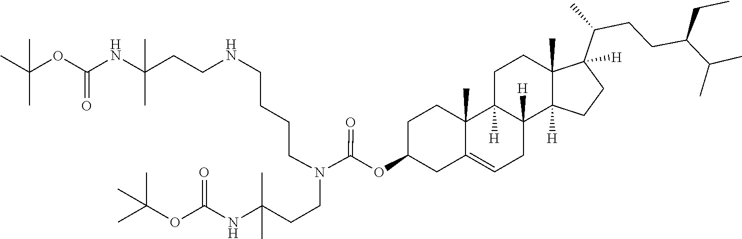

To a solution of di-tert-butyl ((butane-1,4-diylbis(azanediyl))bis(2-methylbutane-4,2-diyl))dicarbamate (0.30 g, 0.65 mmol) in dry toluene (10 mL) set stirring under nitrogen was added triethylamine (0.32 mL, 2.30 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.38 g, 0.65 mmol) was added and the solution was heated to 90° C. and allowed to proceed overnight. The following morning, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate as a light yellow oil (0.36 g, 0.40 mmol, 60.5%). UPLC/ELSD: RT=2.86 min. MS (ES): m/z (MH⁺) 900.3 for C₅₄H₉₈N₄O₆. ¹H NMR (300 MHz, CDCl₃) δ: ppm 5.63 (m, 1H), 5.09 (m, 1H), 5.01 (s, 1H), 4.22 (br. m, 2H), 2.93 (br. m, 4H), 2.40 (t, 2H), 2.32 (t, 2H), 2.05 (br. m, 2H), 1.59 (br. m, 7H), 1.26 (br. m, 13H), 1.14 (s, 20H), 1.02 (d, 16H, J=9 Hz), 0.84 (br. m, 9H), 0.73 (s, 6H), 0.65 (d, 6H, J=6 Hz), 0.56 (q, 10H, J=6 Hz), 0.39 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride

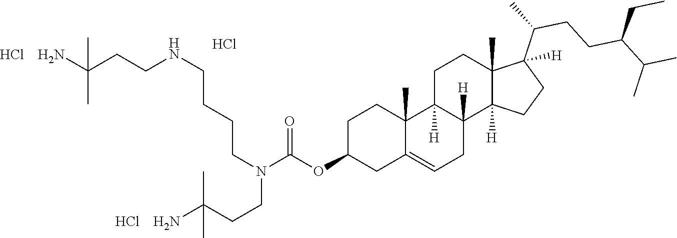

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((3-((tert-butoxycarbonyl)amino)-3-methylbutyl)amino)butyl)carbamate (0.36 g, 0.40 mmol) in isopropanol (7 mL) set stirring under nitrogen was added hydrochloric acid (5N in isopropanol, 0.79 mL, 3.96 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-((3-amino-3-methylbutyl)amino)butyl)carbamate trihydrochloride as a white solid (0.24 g, 0.27 mmol, 69.3%). UPLC/ELSD: RT=1.50 min. MS (ES): m/z (MH⁺) 700.3 for C₄₄H₈₅C₃N₄O₂. ¹H NMR (300 MHz, MeOD) δ: ppm 5.42 (m, 1H), 4.44 (br. m, 1H), 3.94 (m, 1H), 3.48 (br. m, 2H), 3.33 (br. m, 8H), 3.15 (m, 4H), 2.40 (d, 2H, J=3 Hz), 2.12 (br. m, 10H), 1.74 (br. m, 12H), 1.42 (d, 16H, J=6 Hz), 1.18 (d, 11H, J=6 Hz), 1.08 (s, 6H), 0.98 (d, 5H, J=6 Hz), 0.87 (q, 9H, J=6 Hz), 0.75 (s, 3H).

### AQ. Compound SA116: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

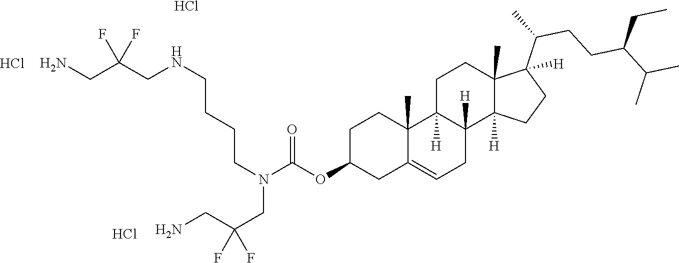

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate

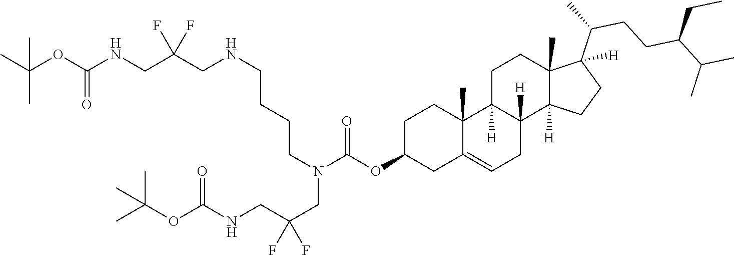

To a solution of di-tert-butyl ((butane-1,4-diylbis(azanediyl))bis(2,2-difluoropropane-3,1-diyl))dicarbamate (0.37 g, 0.78 mmol) in dry toluene (10 mL) set stirring under nitrogen was added triethylamine (0.33 mL, 2.33 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.45 g, 0.78 mmol) was added. The solution was heated to 90° C. and allowed to proceed for 48 h. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in DCM with a 0-80% (70:25:5 DCM/MeOH/concentrated aqueous ammonium hydroxide) gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate as a light yellow oil (0.05 g, 0.06 mmol, 7.3%). UPLC/ELSD: RT=2.77 min. MS (ES): m/z (MH⁺) 916.3 for C₅₀H₈₆F₄N₄O₆. ¹H NMR (300 MHz, CDCl₃) δ: ppm 5.61 (br. m, 1H), 5.41 (br. m, 1H), 5.00 (br. m, 1H), 4.55 (br. m, 1H), 3.65 (br. m, 4H), 3.33 (br. m, 2H), 2.97 (t, 2H), 2.69 (t, 1H), 2.36 (br. m, 2H), 1.87 (br. m, 4H), 1.59 (br. m, 7H), 1.46 (s, 17H), 1.14 (br. m, 14H), 1.04 (s, 5H), 0.95 (d, 4H, J=6 Hz), 0.86 (q, 8H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride

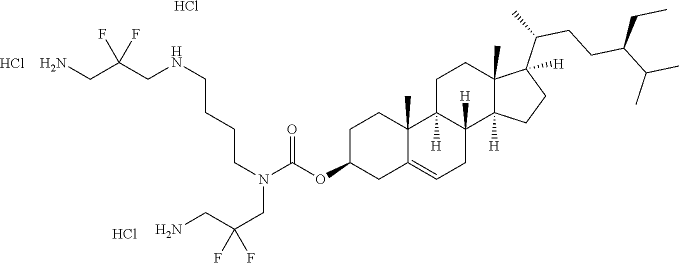

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-2,2-difluoropropyl)(4-((3-((tert-butoxycarbonyl)amino)-2,2-difluoropropyl)amino)butyl)carbamate (0.05 g, 0.06 mmol) in isopropanol (5 mL) set stirring under nitrogen was added hydrochloric acid (5N in isopropanol, 0.11 mL, 0.57 mmol) dropwise. The solution was heated to 40° C. and allowed to proceed overnight. The following morning, dry acetonitrile (10 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-2,2-difluoropropyl)(4-((3-amino-2,2-difluoropropyl)amino)butyl)carbamate trihydrochloride as a white solid (0.04 g, 0.04 mmol, 77.1%). UPLC/ELSD: RT=1.63 min. MS (ES): m/z (MH⁺) 716.1 for C₄₀H₇₃Cl₃F₄N₄O₂. ¹H NMR (300 MHz, MeOD) δ: ppm 5.32 (m, 1H), 4.38 (br. m, 1H), 3.81 (br. m, 6H), 3.33 (br. m, 4H), 3.22 (s, 5H), 3.08 (br. m, 2H), 2.28 (d, 2H, J=3 Hz), 1.93 (br. m, 5H), 1.54 (br. m, 9H), 1.26 (br. m, 6H), 1.06 (d, 6H, J=6 Hz), 0.97 (s, 5H), 0.87 (d, 4H, J=6 Hz), 0.77 (q, 7H, J=6 Hz), 0.63 (s, 3H).

### AR. Compound SA117: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

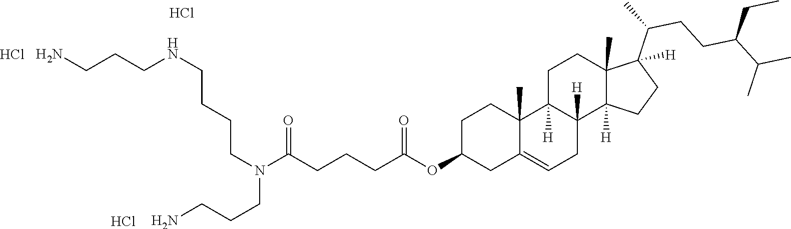

### Step 1: 5-(((3S,8S, 9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid

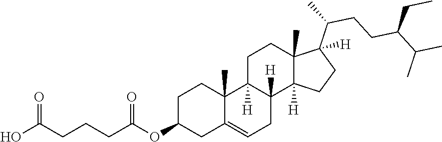

To a solution of sitosterol (2.50 g, 5.71 mmol) in acetone (30 mL) stirring under nitrogen was added glutaric anhydride (1.17 g, 10.28 mmol) and triethylamine (1.43 mL, 10.28 mmol). The reaction mixture was refluxed at 56° C., turning from a white slurry to a colorless clear solution, and allowed to proceed at reflux for 3 days. The solution was then cooled to room temperature, concentrated under vacuum, and taken up in 150 mL dichloromethane. This was then washed with 0.5 M HCl (1×100 mL), saturated aqueous ammonium chloride (1×100 mL), and water (1×100 mL), dried over sodium sulfate, filtered, and concentrated to a white solid. The solid was taken up in dichloromethane and purified on silica with a 0-80% ethyl acetate gradient in hexanes to give 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid as a white solid (2.33 g, 4.40 mmol, 77.1%). UPLC/ELSD: RT: 3.30 min. MS (ES): m/z (MH⁺) 529.8 for C₃₄H₅₆O₄. ¹H NMR (300 MHz, CDCl₃) δ: ppm 5.38 (m, 1H), 3.53 (m, 1H), 2.31 (br. m, 3H), 2.07 (br. m, 3H), 1.98 (br. m, 3H), 1.50 (br. m, 7H), 1.26 (br. m, 12H), 1.03 (s, 5H), 0.93 (d, 6H, J=6 Hz), 0.85 (q, 10H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate

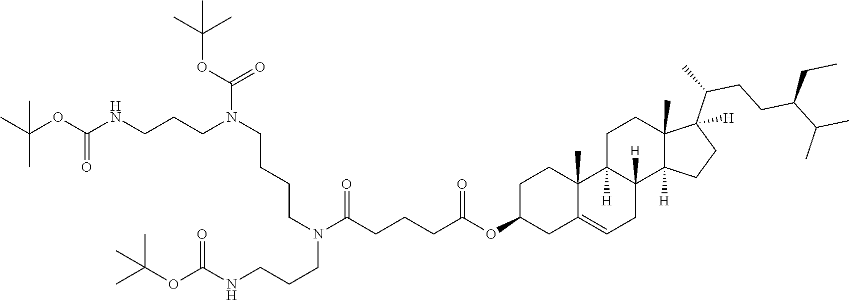

To a solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (1.50 g, 2.81 mmol) in dry DCM (25 mL) stirring under nitrogen was added tert-butyl (3-((tert-butoxycarbonyl)amino)propyl)(4-((3-((tert-butoxycarbonyl)amino)propyl)amino)butyl)carbamate (1.41 g, 2.81 mmol), dimethylaminopyridine (0.04 g, 0.28 mmol), and 1-ethyl-3-(3-dimethylaminopropyl)carbodiimide hydrochloride (1.09 g, 5.62 mmol). The resulting solution was cooled to 0° C. and diisopropylethylamine (1.49 mL, 8.42 mmol) was added dropwise. The mixture was allowed to gradually warm to room temperature and proceed for 48 h. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-80% (70:25:5 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate as a light yellow oil (0.41 g, 0.40 mmol, 14.3%). UPLC/ELSD: RT: 3.29 min. MS (ES): m/z (MH⁺) 1014.5 for C₄₉H₁₀₄N₄O₉. ¹H NMR (300 MHz, CDCl₃) δ: ppm 5.41 (m, 2H), 4.52 (br. m, 1H), 4.14 (m, 1H), 3.16 (br. m, 13H), 2.29 (br. m, 7H), 1.75 (br. m, 21H), 1.35 (d, 33H, J=6 Hz), 1.09 (br. m, 12H), 0.93 (s, 7H), 0.85 (d, 5H, J=6 Hz), 0.76 (q, 9H, J=6 Hz), 0.59 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride

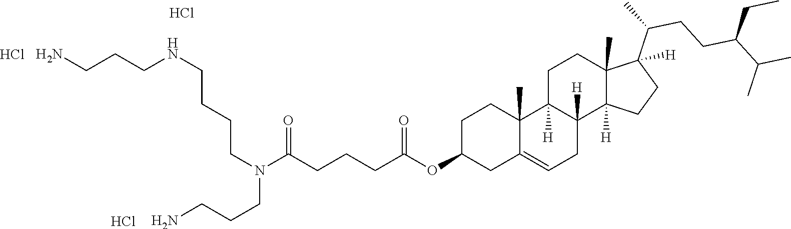

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4,15-dioxo-3-oxa-5,9,14-triazanonadecan-19-oate (0.41 g, 0.40 mmol) in isopropanol (5 mL) set stirring under nitrogen was added hydrochloric acid (5N in isopropanol, 0.80 mL, 4.01 mmol) dropwise. The solution was heated to 40° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminopropyl)(4-((3-aminopropyl)amino)butyl)amino)-5-oxopentanoate trihydrochloride as a white solid (0.24 g, 0.27 mmol, 66.1%). UPLC/ELSD: RT=1.64 min. MS (ES): m/z (MH⁺) 714.3 for C₄₄H₈₃Cl₃N₄O₃. ¹H NMR (300 MHz, MeOD) δ: ppm 5.40 (m, 1H), 4.56 (br. m, 1H), 3.95 (m, 1H), 3.52 (br. m, 3H), 3.33 (s, 3H), 3.15 (br. m, 6H), 2.42 (br. m, 5H), 1.91 (br. m, 10H), 1.54 (br. m, 7H), 1.32 (br. m, 7H), 1.17 (d, 4H, J=6 Hz), 1.06 (s, 4H), 0.97 (d, 4H, J=6 Hz), 0.88 (q, 7H, J=6 Hz), 0.74 (s, 3H).

### AS. Compound SA119: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

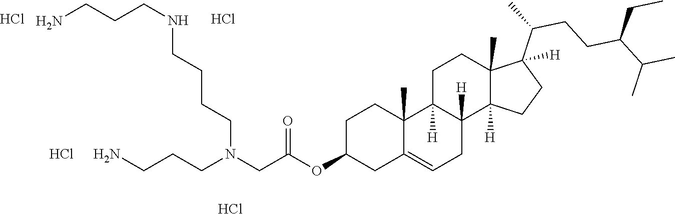

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate

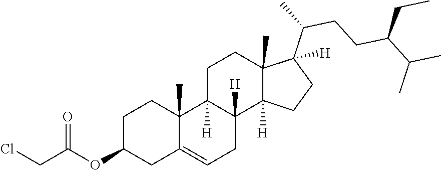

To a solution of sitosterol (2.00 g, 2.57 mmol) in dry dichloroethane (22.84 mL) was added DBU (2.05 mL, 13.70 mmol). The reaction was cooled to 0° C., and a solution of chloroacetyl chloride (0.73 mL, 9.13 mmol) in 5 mL dichloroethane was added to the reaction mixture dropwise, causing a change from a clear colorless solution to a cloudy dark brown mixture. The mixture was allowed to gradually warm to room temperature and stir overnight. The following morning, TLC suggested incomplete reaction progress, so the mixture was again cooled to 0° C. and an additional 0.50 mL DBU and 0.20 mL chloroacetyl chloride were added. The mixture warmed to room temperature, and the reaction was complete by TLC after 2 hours. The mixture was cooled to 0° C. again, and 30 mL water was added. Upon warming to room temperature, the aqueous layer was separated and washed with DCM (3×30 mL), and all organic layers were combined, dried over sodium sulfate, filtered, and concentrated to give a brown oil. The oil was taken up in DCM and purified on silica in hexanes with a 0-20% EtOAc gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate as a white solid (1.40 g, 2.84 mmol, 62.2%). UPLC/ELSD: RT: 3.49 min. MS (ES): m/z (MH⁺) 492.2 for C₃₁H₅₁ClO₂. ¹H NMR (300 MHz, CDCl₃) δ: ppm 5.41 (m, 1H), 4.70 (br. m, 1H), 4.06 (s, 2H), 2.40 (d, 2H, J=6 Hz), 1.93 (br. m, 5H), 1.52 (br. m, 7H), 1.20 (br. m, 11H), 1.05 (s, 6H), 0.96 (d, 5H, J=6 Hz), 0.85 (q, 9H, J=6 Hz), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate

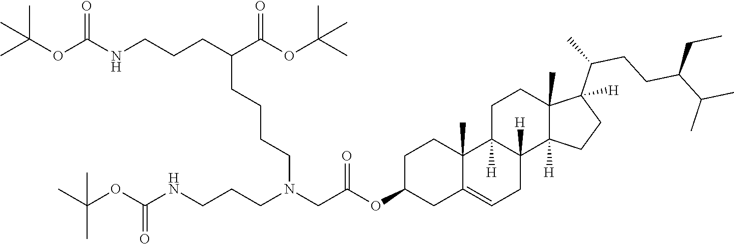

Both (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloroacetate (0.51 g, 1.04 mmol) and tert-butyl (3-((tert-butoxycarbonyl)amino)propyl)(4-((3-((tert-butoxycarbonyl)amino)propyl)amino)butyl)carbamate (0.71 g, 1.41 mmol) were combined in a vial and purged with three cycles of vacuum and nitrogen. Then, they were taken up in dry THE (10.42 mL), and triethylamine (0.29 mL, 2.09 mmol) was added. The mixture was set stirring under nitrogen, heated to 65° C., and allowed to stir for 48 h. Then, the mixture was cooled to room temperature and diluted with ethyl acetate (30 mL) and saturated aq. sodium bicarbonate (30 mL). The aqueous layer was separated and extracted with EtOAc (3×30 mL). All organic layers were combined, washed with brine (1×20 mL), dried over sodium sulfate, filtered, and concentrated to a yellow oil. The oil was taken up in DCM and purified on silica in hexanes with a 0-70% EtOAc gradient. Product-containing fractions were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate as a light yellow oil (0.47 g, 0.49 mmol, 46.8%). UPLC/ELSD: RT: 2.90 min. MS (ES): m/z (MH⁺) 958.4 for C₅₆H₁₀₀N₄O₈. ¹H NMR (300 MHz, CDCl₃) δ: ppm 5.39 (m, 1H), 4.64 (br. m, 1H), 4.14 (m, 1H), 3.26 (br. m, 9H), 2.60 (m, 4H), 2.35 (d, 2H, J=6 Hz), 2.05 (br. m, 6H), 1.65 (br. m, 8H), 1.47 (br. s, 30H), 1.20 (br. m, 11H), 1.03 (s, 5H), 0.95 (d, 5H, J=6 Hz), 0.86 (q, 8H, J=6 Hz), 0.69 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride

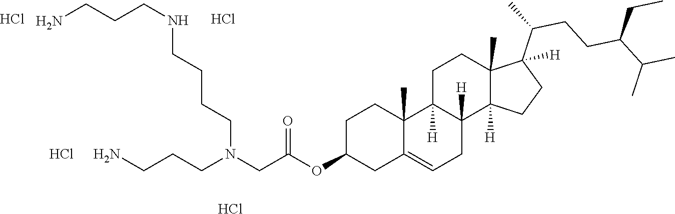

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2-dimethyl-4-oxo-3-oxa-5,9,14-triazahexadecan-16-oate (0.47 g, 0.49 mmol) in isopropanol (7 mL) set stirring under nitrogen was added hydrochloric acid (5N in isopropanol, 1.17 mL, 5.85 mmol) dropwise. The solution was heated to 40° C. and allowed to proceed overnight. The following morning, the solution was cooled to room temperature and dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)glycinate tetrahydrochloride as a white solid (0.36 g, 0.41 mmol, 83.5%). UPLC/ELSD: RT=1.71 min. MS (ES): m/z (MH⁺) 658.1 for C₄₁H₈₀Cl₄N₄O₂. ¹H NMR (300 MHz, MeOD) δ: ppm 5.42 (m, 1H), 4.73 (br. m, 2H), 4.30 (br. m, 2H), 3.93 (m, 1H), 3.32 (br. m, 6H), 3.10 (br. m, 8H), 2.43 (br. s, 2H), 2.17 (br. m, 4H), 2.03 (br. m, 10H), 1.53 (br. m, 8H), 1.31 (br. m, 9H), 1.15 (d, 6H, J=6 Hz), 1.05 (s, 6H), 0.95 (d, 5H, J=6 Hz), 0.86 (q, 9H, J=6 Hz), 0.72 (s, 3H).

### AT. Compound SA120: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride

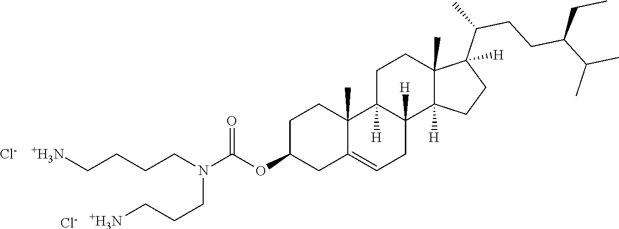

### Step 1: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate

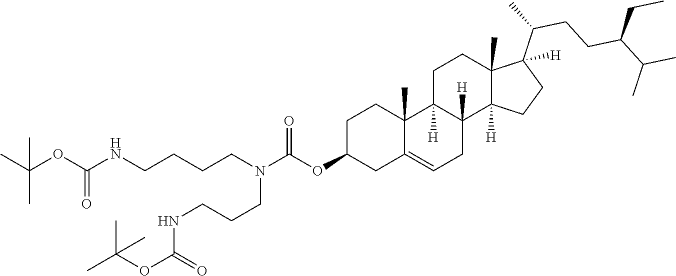

β-Sitosterol 4-nitrophenyl carbonate (0.200 g, 0.345 mmol), tert-butyl N-[3-({4-[(tert-butoxycarbonyl)amino]butyl}amino)propyl]carbamate (0.155 g, 0.448 mmol), and triethylamine (0.15 mL, 1.1 mmol) were combined in toluene (3.5 mL). The reaction mixture stirred at 90° C. and was monitored by LCMS. At 21 h the reaction mixture was cooled to rt, diluted with dichloromethane (20 mL), and washed with 5% aq. NaHCO₃ solution (3×10 mL). The organics were passed through a hydrophobic frit, dried over Na₂SO₄, and concentrated. The crude material was purified via silica gel chromatography (10-50% ethyl acetate in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate (0.210 g, 0.267 mmol, 77.4%) as a white foam. UPLC/ELSD: RT=3.37 min. MS (ES): m/z=787.67 [M+H]+ for C₄₇H₈₃N₃O₆; ¹H NMR (300 MHz, CDCl₃): δ 5.10-5.44 (m, 2H), 4.42-4.89 (m, 2H), 3.01-3.40 (br. m, 8H), 2.22-2.42 (m, 2H), 1.76-2.09 (m, 5H), 0.88-1.75 (br. m, 28H), 1.44 (s, 18H), 1.02 (s, 3H), 0.92 (d, 3H, J=6.4 Hz), 0.78-0.88 (m, 9H), 0.68 (s, 3H).

### Step 2: (3S,8S,9S,10R,13R,14S,17R)-17-((2R,5R)-5-Ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride

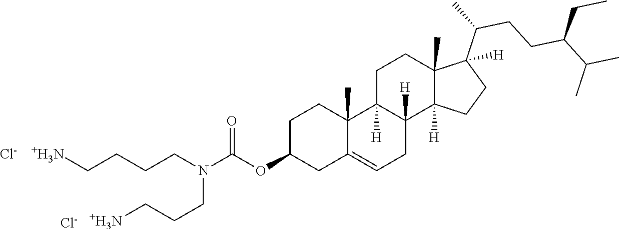

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)amino)butyl)(3-((tert-butoxycarbonyl)amino)propyl)carbamate (0.203 g, 0.258 mmol) in isopropanol (3.0 mL) was added 5-6 N HCl in isopropanol (0.37 mL). The reaction mixture stirred at 40° C. and was monitored by LCMS. At 24 h acetonitrile (9 mL) was added, and the reaction mixture stirred for 5 min. After this time solids were collected by vacuum filtration rinsing with 3:1 acetonitrile/isopropanol to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-aminobutyl)(3-aminopropyl)carbamate dihydrochloride (0.151 g, 0.218 mmol, 84.4%) as a white solid. UPLC/ELSD: RT=1.93 min. MS (ES): m/z=586.67 [M+H]⁺ for C₃₇H₆₇N₃O₂; ¹H NMR (300 MHz, CD₃OD): δ 5.38-5.47 (m, 1H), 4.39-4.55 (m, 1H), 3.28-3.46 (m, 4H), 2.91-3.06 (m, 4H), 2.31-2.47 (m, 2H), 1.83-2.14 (m, 7H), 0.93-1.79 (br. m, 26H), 1.08 (s, 3H), 0.98 (d, 3H, J=6.4 Hz), 0.82-0.93 (m, 9H), 0.75 (s, 3H).

### AU. Compound SA118: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride

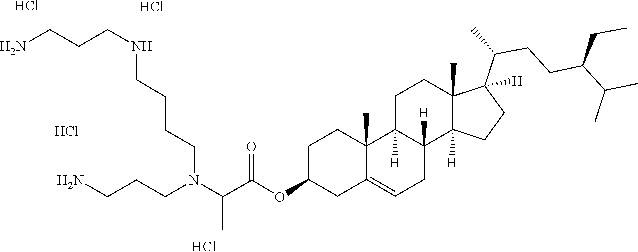

### Step 1: (3S,8S, 9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate

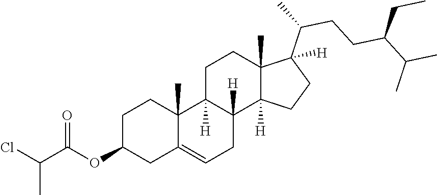

To a solution of β-sitosterol (1.25 g, 2.85 mmol) in dry DCM (15 mL) stirring under nitrogen was added 1,8-diazabicyclo[5.4.0]undec-7-ene (1.28 mL, 8.56 mmol). The reaction was cooled to 0° C., and a solution of 2-chloropropanoyl chloride (0.55 mL, 5.71 mmol) in 5 mL dry DCM was added dropwise over 20 minutes causing the solution to turn from a clear colorless mixture to a cloudy dark brown mixture. The reaction mixture was allowed to gradually warm to room temperature and proceed overnight. The following morning the reaction appeared to be incomplete via thin layer chromatography (7:3 hexanes/ethyl acetate, PMA stain), so the reaction mixture was again cooled to 0° C., and an additional 0.50 mL of 1,8-diazabicyclo[5.4.0]undec-7-ene was added. The reaction was allowed to warm to room temperature. After an hour, the reaction appeared complete by TLC. The reaction mixture was cooled to 0° C. and quenched with 20 mL of water. After the mixture warmed to room temperature, the layers were separated, and the aqueous layer was extracted with DCM (3×30 mL). All organic layers were combined, dried over sodium sulfate, filtered, and concentrated to a dark brown oil. The oil was taken up and purified on silica in hexanes with a gradient of 0-20% ethyl acetate. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate as a white solid (1.12 g, 2.22 mmol, 77.7%). UPLC/ELSD: RT=3.39 min. MS (ES): m/z (MH⁺) 506.2 for C₃₂H₅₃ClO₂. ¹H NMR (300 MHz, CDCl₃) δ: ppm 5.44 (m, 1H), 4.48 (br. m, 1H), 3.95 (m, 1H), 3.33 (br. m, 5H), 3.11 (br. m, 8H), 2.45 (br. m, 7H), 1.99 (br. m, 7H), 1.68 (br. m, 11H), 1.37 (br. m, 9H), 1.15 (d, 8H, J=6 Hz), 1.08 (s, 6H), 0.95 (d, 5H, J=6 Hz), 0.86 (q, 9H), 0.74 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate

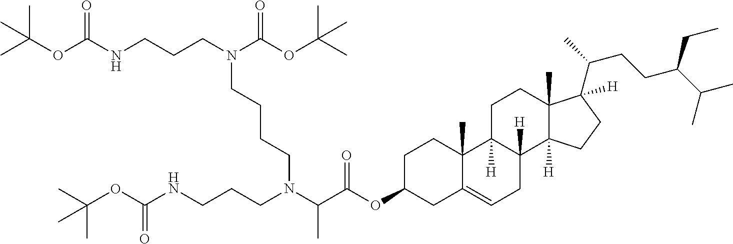

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 2-chloropropanoate (1.12 g, 2.22 mmol) and tert-butyl N-{3-[(tert-butoxycarbonyl)amino]propyl}-N-[4-({3-[(tert-butoxycarbonyl)amino]propyl}amino)butyl]carbamate (1.34 g, 2.66 mmol) in dry THE (22 mL) stirring under nitrogen was added triethylamine (0.62 mL, 4.43 mmol). The mixture was heated to 65° C. and allowed to proceed for a week, during which very little product was formed as monitored by LCMS. After a week, the mixture was cooled to room temperature and concentrated to an oil in vacuo. The oil was taken up in DCM and purified on silica in hexanes with a 0-50% ethyl acetate gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate as a light yellow oil (0.09 g, 0.10 mmol, 4.4%). UPLC/ELSD: RT=2.79 min. MS (ES): m/z (MH⁺) 972.5 for C₅₇H₁₀₂N₄O₈. ¹H NMR (300 MHz, CDCl₃) δ: ppm 5.31 (m, 1H), 4.54 (br. m, 1H), 3.39 (m, 1H), 3.07 (br. m, 7H), 2.52 (br. m, 4H), 2.23 (d, 2H, J=6 Hz), 1.77 (br. m, 6H), 1.54 (br. m, 9H), 1.36 (d, 30H, J=9 Hz), 1.17 (br. m, 14H), 0.95 (s, 5H), 0.86 (d, 5H, J=6 Hz), 0.75 (q, 8H), 0.61 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride

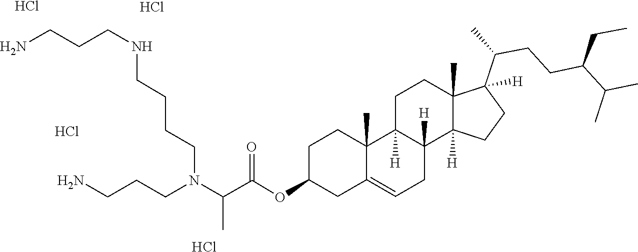

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 9-(tert-butoxycarbonyl)-14-(3-((tert-butoxycarbonyl)amino)propyl)-2,2,15-trimethyl-4-oxo-3-oxa-5,9,14-triazahexadecan-16-oate (0.09 g, 0.10 mmol) in isopropanol (5 mL) set stirring under nitrogen was added hydrochloric acid (5 N in isopropanol, 0.19 mL, 0.97 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl N-(3-aminopropyl)-N-(4-((3-aminopropyl)amino)butyl)alaninate tetrahydrochloride as a white solid (0.07 g, 0.07 mmol, 76.9%). UPLC/ELSD: RT=1.61 min. MS (ES): m/z (MH⁺) 672.2 for C₄₂H₈₂Cl₄N₄O₂. ¹H NMR (300 MHz, MeOD) δ: ppm 5.44 (m, 1H), 4.48 (br. m, 1H), 3.95 (m, 1H), 3.32 (s, 5H), 3.11 (br. m, 8H), 2.27 (br. m, 7H), 1.99 (br. m, 7H), 1.68 (br. m, 11H), 1.37 (br. m, 9H), 1.15 (d, 8H, J=6 Hz), 1.08 (s, 6H), 0.95 (d, 5H, J=6 Hz), 0.86 (q, 9H), 0.74 (s, 3H).

### AV. Compound SA121: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

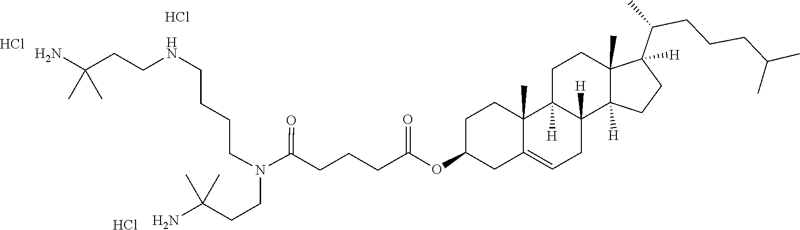

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate

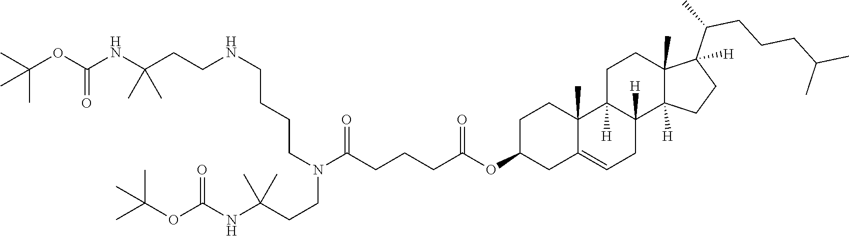

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.23 g, 0.45 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.23 g, 0.49 mmol), dimethylaminopyridine (0.01 g, 0.09 mmol), and 1-ethyl-3-(3-dimethylaminopropyl)carbodiimide hydrochloride (0.17 g, 0.89 mmol). The resulting solution was cooled to 0° C. and diisopropylethylamine (0.24 mL, 1.34 mmol) was added dropwise. The mixture was allowed to gradually warm to room temperature and proceed overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-80% (70:25:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate as a light yellow oil (0.09 g, 0.09 mmol, 20.2%). UPLC/ELSD: RT: 2.74 min. MS (ES): m/z (MH⁺) 942.4 for C₅₆H₁₀₀N₄O₇. ¹H NMR (300 MHz, CDCl₃) δ: ppm 5.30 (m, 1H), 4.52 (br. m, 2H), 3.23 (m, 4H), 2.93 (s, 1H), 2.61 (t, 2H) 2.54 (t, 2H), 2.29 (br. m, 6H), 1.87 (br. m, 10H), 1.62 (m, 3H), 1.49 (m, 6H), 1.36 (d, 24H, J=3 Hz), 1.23 (br. m, 17H), 1.06 (br. m, 7H), 0.94 (s, 7H), 0.85 (d, 4H, J=9 Hz), 0.81 (d, 7H, J=9 Hz), 0.61 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

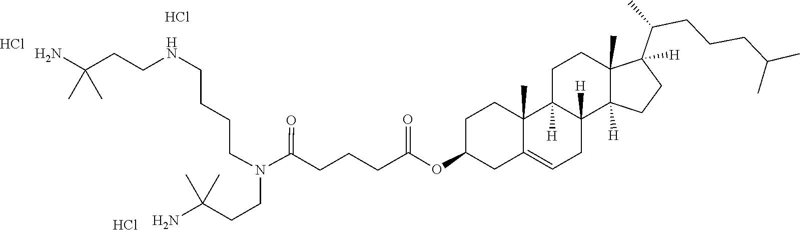

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate (0.06 g, 0.06 mmol) in isopropanol (5 mL) set stirring under nitrogen was added hydrochloric acid (5 N in isopropanol, 0.12 mL, 0.61 mmol) dropwise. The solution was heated to 42° C. and allowed to proceed overnight. The following morning, dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride as a white solid (0.03 g, 0.04 mmol, 58.0%). UPLC/ELSD: RT=1.62 min. MS (ES): m/z (MH⁺) 742.3 for C₄₆H₈₇Cl₃N₄O₃. ¹H NMR (300 MHz, MeOD) δ: ppm 5.42 (m, 1H), 4.57 (br. m, 1H), 3.48 (m, 4H), 3.33 (br. m, 3H), 3.16 (br. m, 4H), 2.48 (br. m, 5H), 2.14 (m, 2H), 1.91 (br. m, 10H), 1.54 (br. m, 6H), 1.42 (br. m, 14H), 1.16 (m, 6H), 1.06 (s, 5H), 0.97 (d, 3H, J=6 Hz), 0.91 (q, 5H, J=6 Hz), 0.74 (s, 3H).

### AW. Compound SA122: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride

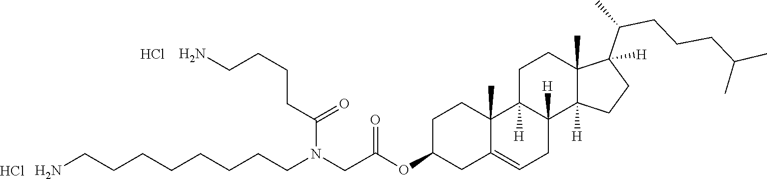

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate

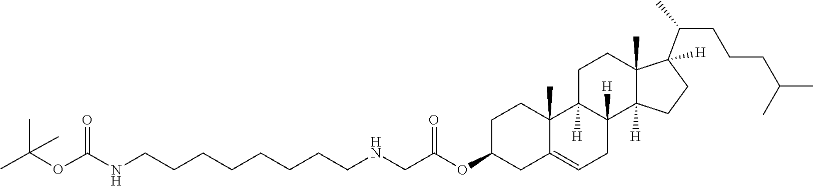

Tert-butyl N-(8-aminooctyl)carbamate (0.994 g, 4.07 mmol), cholesteryl chloroacetate (1.000 g, 2.159 mmol), potassium iodide (0.072 g, 0.43 mmol) and potassium carbonate (0.597 g, 4.32 mmol) were combined in dioxane (15 mL) in a sealed tube. The reaction mixture was monitored by LCMS. The reaction mixture was irradiated with microwaves at 140° C. for 3 h while stirring. The reaction mixture was irradiated with microwaves at 150° C. for 3 h while stirring, cooled to rt, and filtered through a pad of Celite rinsing with EtOAc. The filtrate was concentrated and then taken up in DCM (100 mL). The organics were washed with water, passed through a hydrophobic frit, dried over Na₂SO₄, and concentrated. The crude material was purified via silica gel chromatography (30-70% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.993 g, 1.48 mmol, 68.5%) as a viscous, amber oil. UPLC/ELSD: RT=2.56 min. MS (ES): m/z=672.06 [M+H]⁺ for C₄₂H₇₄N₂O₄. ¹H NMR (300 MHz, CDCl₃): δ 5.35-5.43 (m, 1H), 4.60-4.76 (m, 1H), 4.50 (br. s, 1H), 3.46 (s, 2H), 2.99-3.17 (m, 2H), 2.69 (t, 2H, J=7.3 Hz), 2.28-2.40 (m, 2H), 1.72-2.08 (m, 5H), 0.93-1.71 (br. m, 33H), 1.44 (s, 9H), 1.02 (s, 3H), 0.91 (d, 3H, J=6.5 Hz), 0.87 (d, 3H, J=6.5 Hz), 0.86 (d, 3H, J=6.6 Hz), 0.68 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate

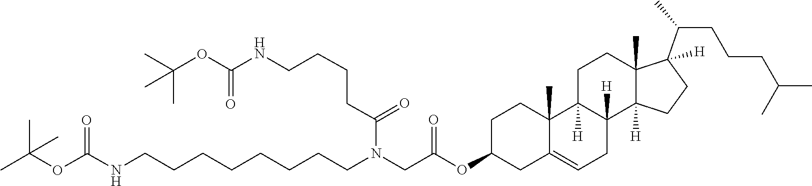

(3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.150 g, 0.224 mmol), 5-[(tert-butoxycarbonyl)amino]pentanoic acid (0.058 g, 0.268 mmol), and DMAP (cat.) were combined in DCM (3.0 mL). The reaction mixture was cooled to 0° C. in an ice bath and then 1-ethyl-3-(3-dimethylaminopropyl)carbodiimide hydrochloride (0.064 g, 0.34 mmol) was added. The reaction mixture stirred at rt and was monitored by LCMS. At 20 h, the reaction mixture was cooled to 0° C. in an ice bath, and then water (3 mL) was added. The biphasic mixture was diluted with DCM (5 mL). The layers were separated, and the aqueous was extracted with DCM (5 mL). The combined organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate (0.185 g, 0.213 mmol, 95.1%) as a clear oil. UPLC/ELSD: RT=3.27 min. MS (ES): m/z=872.43 [M+H]⁺ for C₅₂H₉₁N₃O₇. ¹H NMR (300 MHz, CDCl₃): δ 5.32-5.47 (m, 1H), 4.29-4.78 (m, 3H), 3.92-4.06 (m, 2H), 3.23-3.42 (m, 2H), 3.01-3.21 (m, 4H), 2.18-2.45 (m, 4H), 1.05-2.10 (br. m, 60H), 1.01 (s, 3H), 0.91 (d, 3H, J=6.2 Hz), 0.87 (d, 6H, J=6.5 Hz), 0.68 (s, 3H).

### Step 3: (3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride

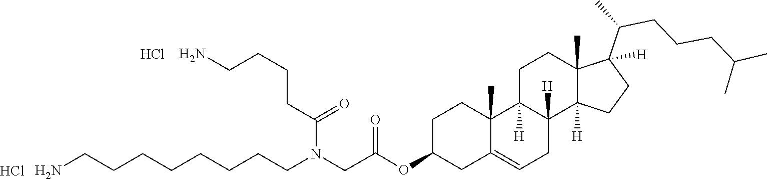

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-((tert-butoxycarbonyl)amino)octyl)-N-(5-((tert-butoxycarbonyl)amino)pentanoyl)glycinate (0.237 g, 0.272 mmol) in iPrOH (3.5 mL) was added 5-6 N HCl in iPrOH (0.39 mL). The reaction mixture was stirred at 40° C. and was monitored by LCMS. At 16 h, the reaction mixture was cooled to rt, and then ACN (10.5 mL) was added. Solids were collected by vacuum filtration rinsing with 3:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-(5-aminopentanoyl)glycinate dihydrochloride (0.150 g, 0.185 mmol, 68.1%) as a white solid. UPLC/ELSD: RT=1.94 min. MS (ES): m/z=336.11 [M+2H]²⁺ for C₄₂H₇₅N₃O₃. ¹H NMR (300 MHz, DMSO): δ 7.90 (br. s, 6H), 5.31-5.40 (m, 1H), 4.39-4.61 (m, 1H), 3.89-4.24 (m, 2H), 3.18-3.39 (m, 2H), 2.65-2.88 (m, 4H), 2.13-2.41 (m, 4H), 1.70-2.05 (m, 5H), 0.91-1.67 (br. m, 37H), 0.98 (s, 3H), 0.89 (d, 3H, J=6.3 Hz), 0.84 (d, 6H, J=6.5 Hz), 0.65 (s, 3H).

### AX. Compound SA123: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride

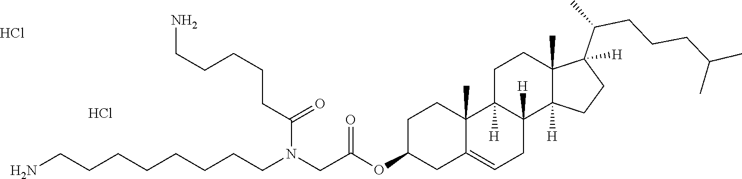

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate

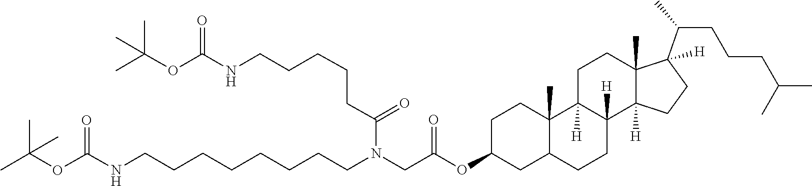

(3S,8S,9S,10R,13R,14S,17R)-10,13-Dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.200 g, 0.298 mmol), 6-[(tert-butoxycarbonyl)amino]hexanoic acid (0.090 g, 0.39 mmol), and DMAP (cat.) were combined in DCM (4.0 mL). The reaction mixture was cooled to 0° C. in an ice bath, and 1-ethyl-3-(3-dimethylaminopropyl)carbodiimide hydrochloride (0.086 g, 0.45 mmol) was added. The reaction mixture was stirred at rt and was monitored by LCMS. At 20 h, the reaction mixture was cooled to 0° C. in an ice bath, and then water (4 mL) was added. The biphasic mixture was diluted with DCM (5 mL). The layers were separated, and the aqueous was extracted with DCM (5 mL). The combined organics were passed through a hydrophobic frit, dried over Na₂SO₄, and concentrated. The crude material was purified via silica gel chromatography (20-60% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate (0.260 g, 0.294 mmol, 98.6%) as a clear oil. UPLC/ELSD: RT=3.28 min. MS (ES): m/z=885.39 [M+H]⁺ for C₅₃H₉₃N₃O₇. ¹H NMR (300 MHz, CDCl₃): δ 5.27-5.51 (m, 1H), 4.28-4.76 (m, 3H), 3.94-4.05 (m, 2H), 3.23-3.41 (m, 2H), 3.02-3.20 (m, 4H), 2.16-2.43 (m, 4H), 0.93-2.11 (br. m, 65H), 0.91 (d, 3H, J=6.5 Hz), 0.87 (d, 3H, J=6.6 Hz), 0.86 (d, 3H, J=6.6 Hz), 0.63-0.75 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride

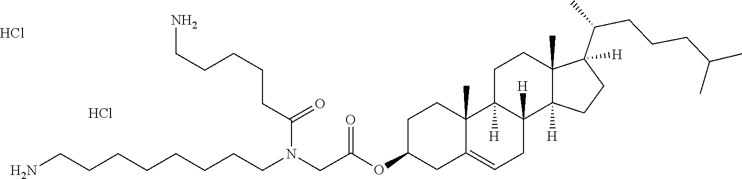

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-((tert-butoxycarbonyl)amino)hexanoyl)-N-(8-((tert-butoxycarbonyl)amino)octyl)glycinate (0.251 g, 0.284 mmol) in iPrOH (4.0 mL) was added 5-6 N HCl in iPrOH (0.40 mL). The reaction mixture stirred at 40° C. and was monitored by LCMS. At 16 h, the reaction mixture was cooled to rt, then ACN (12 mL) was added. Solids were collected by vacuum filtration rinsing with 3:1 ACN/iPrOH to afford (3 S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(6-aminohexanoyl)-N-(8-aminooctyl)glycinate dihydrochloride (0.163 g, 0.198 mmol, 69.8%) as a white solid. UPLC/ELSD: RT=1.97 min. MS (ES): m/z=343.02 [M+2H]²⁺ for C₄₃H₇₇N₃O₃. ¹H NMR (300 MHz, CD₃OD): δ 5.37-5.47 (m, 1H), 4.51-4.70 (m, 1H), 4.02-4.25 (m, 2H), 3.35-3.49 (m, 2H), 2.87-3.01 (m, 4H), 2.28-2.56 (m, 4H), 1.79-2.14 (m, 5H), 0.99-1.78 (br. m, 39H), 1.07 (s, 3H), 0.96 (d, 3H, J=6.4 Hz), 0.90 (d, 6H, J=6.6 Hz), 0.74 (s, 3H).

### AY. Compound SA124: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride

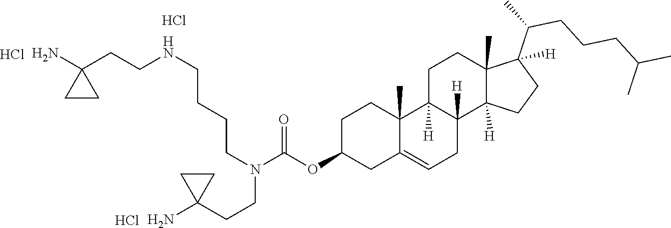

### Step 1: tert-butyl (1-(2-((2-nitrophenyl)sulfonamido)ethyl)cyclopropyl)carbamate

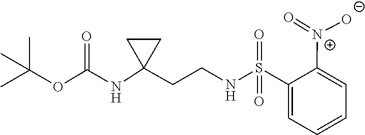 [SMILES: CC(C)(C)OC(=O)NC1(CCNS(=O)(=O)c2ccccc2[N+](=O)[O-])CC1]

To a solution of tert-butyl N-[1-(2-aminoethyl)cyclopropyl]carbamate (2.00 g, 9.49 mmol) in dry DCM (25 mL) set stirring under nitrogen was added triethylamine (2.64 mL, 18.98 mmol). The solution was cooled to 0° C., and then a solution of 2-nitrobenzenesulfonyl chloride (2.31 g, 10.44 mmol) in 25 mL dry DCM was added dropwise over 30 minutes. The reaction was allowed to proceed at 0° C. for an hour and then at room temperature for an additional three hours. Following, the mixture was diluted with an additional 10 mL DCM, washed with 1M aqueous sodium bicarbonate (2×15 mL), water (1×15 mL), 10% aqueous citric acid (2×15 mL), water (1×15 mL), and brine (2×15 mL), dried over sodium sulfate, filtered, and concentrated to give tert-butyl (1-(2-((2-nitrophenyl)sulfonamido)ethyl)cyclopropyl)carbamate as a white solid (3.73 g, 9.70 mmol, quantitative). UPLC/ELSD: RT=0.59 min. MS (ES): m/z (MH⁺) 386.4 for C₁₆H₂₃N₃O₆S. ¹H NMR (300 MHz, CDCl₃) δ: ppm 8.13 (m, 1H), 7.84 (m, 1H), 7.73 (m, 2H), 6.45 (br. s, 1H), 4.89 (br. s, 1H), 3.27 (q, 2H), 1.73 (t, 2H), 1.38 (s, 9H), 0.82 (br. m, 2H), 0.69 (br. s, 2H).

### Step 2: di-tert-butyl (((butane-1,4-diylbis(azanediyl))bis(ethane-2,1-diyl))bis(cyclopropane-1,1-diyl))dicarbamate

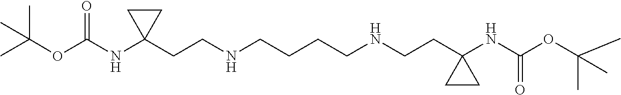 [SMILES: CC(C)(C)OC(=O)NC1(CCNCCCCNCCC2(NC(=O)OC(C)(C)C)CC2)CC1]

To a solution of tert-butyl (1-(2-((2-nitrophenyl)sulfonamido)ethyl)cyclopropyl)carbamate (3.74 g, 9.70 mmol) in dry DMF (50 mL) set stirring under nitrogen was added potassium carbonate (3.89 g, 28.16 mmol) and 1,4-diiodobutane (0.61 mL, 4.62 mmol). The solution was heated to 40° C. and allowed to proceed overnight. The following morning, benzyl bromide (0.46 mL, 3.83 mmol) was added, and the reaction was allowed to proceed at room temperature for 8 h. Then thiophenol (1.82 mL, 17.78 mmol), potassium carbonate (1.91 g, 13.85 mmol), and an additional 10 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×5 mL), dried over potassium carbonate, filtered, and concentrated to an oil. The oil was taken up again in DCM and purified via silica gel chromatography in DCM with a 0-60% (70:20:10 DCM/MeOH/concentrated aqueous ammonium hydroxide) gradient. Fractions containing product were combined and concentrated to give di-tert-butyl (((butane-1,4-diylbis(azanediyl))bis(ethane-2,1-diyl))bis(cyclopropane-1,1-diyl))dicarbamate as a colorless oil (0.47 g, 1.04 mmol, 22.5%). UPLC/ELSD: RT=0.35 min. MS (ES): m/z (MH⁺) 455.6 for C₂₄H₄₆N₄O₄. ¹H NMR (300 MHz, CDCl₃) δ: ppm 5.03 (m, 2H), 2.76 (t, 4H), 2.64 (m, 4H), 1.72 (m, 4H), 1.59 (m, 4H), 1.44 (s, 17H), 0.80 (m, 4H), 0.66 (m, 4H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate

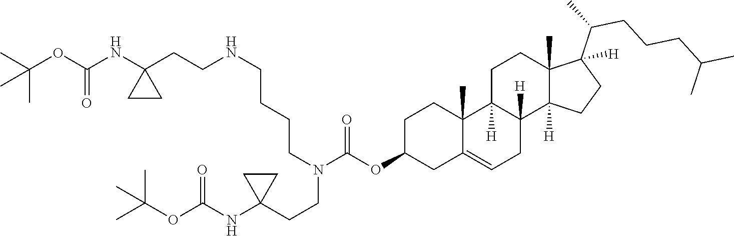

To a solution of di-tert-butyl (((butane-1,4-diylbis(azanediyl))bis(ethane-2,1-diyl))bis(cyclopropane-1,1-diyl))dicarbamate (0.47 g, 1.03 mmol) in dry toluene (20 mL) set stirring under nitrogen was added triethylamine (0.43 mL, 3.08 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.57 g, 1.03 mmol) was added, and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-70% (70:25:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate as a colorless oil (0.35 g, 0.41 mmol, 39.4%). UPLC/ELSD: RT=2.65 min. MS (ES): m/z (MH⁺) 868.3 for C₅₂H₉₀N₄O₆. ¹H NMR (300 MHz, CDCl₃) δ: ppm 5.80 (m, 1H), 5.40 (m, 1H), 4.96 (br. m, 2H), 4.50 (m, 1H), 3.42 (m, 2H), 3.15 (m, 2H), 2.74 (t, 2H), 2.61 (m, 2H), 2.34 (m, 2H), 2.00 (m, 5H), 1.70 (m, 4H), 1.56 (br. m, 8H), 1.44 (s, 18H), 1.15 (br. m, 10H), 1.03 (s, 5H), 0.94 (d, 4H, J=6 Hz), 0.89 (d, 5H, J=6 Hz), 0.69 (br. m, 11H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride

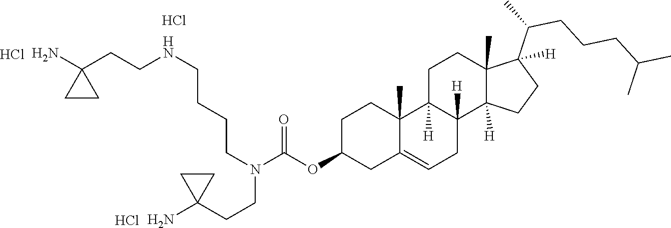

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-((tert-butoxycarbonyl)amino)cyclopropyl)ethyl)(4-((2-(1-((tert-butoxycarbonyl)amino)cyclopropyl)ethyl)amino)butyl)carbamate (0.35 g, 0.41 mmol) in isopropanol (10 mL) set stirring under nitrogen was added hydrochloric acid (5 N in isopropanol, 0.81 mL, 4.05 mmol) dropwise. The solution was heated to 40° C. and allowed to proceed overnight. The following morning, the mixture was cooled to room temperature, and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (2-(1-aminocyclopropyl)ethyl)(4-((2-(1-aminocyclopropyl)ethyl)amino)butyl)carbamate trihydrochloride as a white solid (0.27 g, 0.32 mmol, 80.2%). UPLC/ELSD: RT=1.60 min. MS (ES): m/z (MH⁺) 668.7 for C₄₂H₇₇Cl₃N₄O₂. ¹H NMR (300 MHz, MeOD) δ: ppm 5.41 (m, 1H), 4.46 (br. m, 1H), 3.93 (br. m, 1H), 3.53 (m, 2H), 3.33 (m, 6H), 3.11 (m, 2H), 2.40 (m, 2H), 2.15 (br. m, 4H), 1.93 (br. m, 5H), 1.55 (br. m, 15H), 1.18 (br. m, 11H), 1.08 (m, 8H), 0.97 (m, 7H), 0.89 (m, 7H), 0.74 (s, 3H).

### AZ. Compound SA125: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

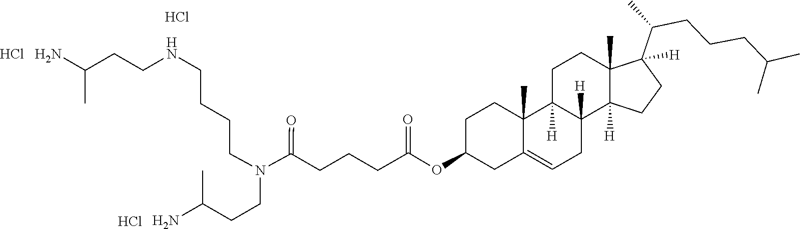

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate

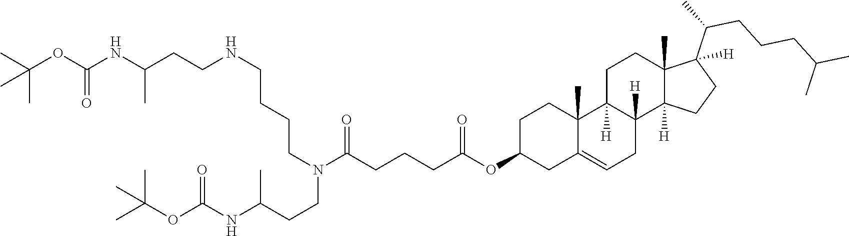

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.30 g, 0.59 mmol) in dry DCM (15 mL) stirring under nitrogen was added di-tert-butyl ((butane-1,4-diylbis(azanediyl))bis(butane-4,2-diyl))dicarbamate (0.77 g, 1.78 mmol), dimethylaminopyridine (0.15 g, 1.19 mmol), and 1-ethyl-3-(3-dimethylaminopropyl)carbodiimide hydrochloride (0.23 g, 1.19 mmol). The mixture was allowed to stir at room temperature and proceed overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate as a light yellow oil (0.18 g, 0.20 mmol, 32.9%). UPLC/ELSD: RT: 2.50 min. MS (ES): m/z (MH⁺) 914.4 for C₅₄H₉₆N₄O₇. ¹H NMR (300 MHz, CDCl₃) δ: ppm 5.33 (m, 1H), 4.82 (br. m, 3H), 3.62 (br. m, 2H), 3.24 (m, 4H), 2.55 (m, 4H), 2.31 (m, 6H), 1.89 (m, 7H), 1.54 (m, 12H), 1.39 (s, 20H), 1.28 (m, 6H), 1.11 (d, 12H, J=6 Hz), 0.97 (s, 6H), 0.87 (d, 4H, J=6 Hz), 0.82 (d, 6H, J=6 Hz), 0.63 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

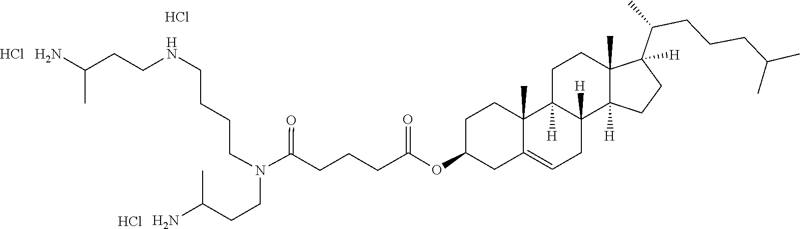

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate (0.18 g, 0.20 mmol) in isopropanol (5 mL) set stirring under nitrogen was added hydrochloric acid (5 N in isopropanol, 0.39 mL, 1.95 mmol) dropwise. The solution was heated to 40° C. and allowed to proceed overnight. The following morning, the mixture was cooled to room temperature and dry acetonitrile (15 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride as a white solid (0.07 g, 0.08 mmol, 40.1%). UPLC/ELSD: RT=1.60 min. MS (ES): m/z (MH⁺) 668.7 for C₄₂H₇₇C₁₃N₄O₂. ¹H NMR (300 MHz, MeOD) δ: ppm 5.41 (m, 1H), 4.46 (br. m, 1H), 3.93 (br. m, 1H), 3.53 (m, 2H), 3.33 (m, 6H), 3.11 (m, 2H), 2.40 (m, 2H), 2.15 (br. m, 4H), 1.93 (br. m, 5H), 1.55 (br. m, 15H), 1.18 (br. m, 11H), 1.08 (m, 8H), 0.97 (m, 7H), 0.89 (m, 7H), 0.74 (s, 3H).

### BA. Compound SA126: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride

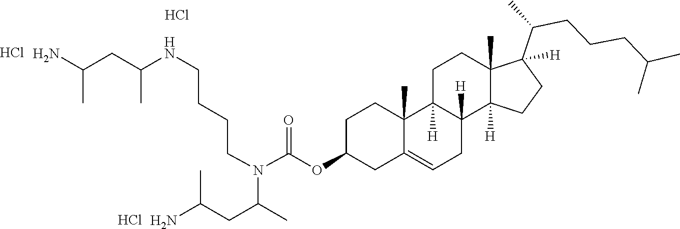

### Step 1: tert-butyl (4-((2-nitrophenyl)sulfonamido)pentan-2-yl)carbamate

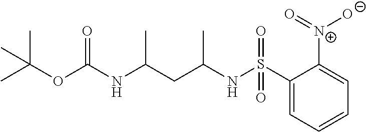 [SMILES: CC(CC(C)NS(=O)(=O)c1ccccc1[N+](=O)[O-])NC(=O)OC(C)(C)C]

To a solution of tert-butyl (4-aminopentan-2-yl)carbamate (2.50 g, 11.74 mmol) in dry DCM (50 mL) set stirring under nitrogen was added triethylamine (3.27 mL, 23.48 mmol). The solution was cooled to 0° C., and then a solution of 2-nitrobenzenesulfonyl chloride (2.86 g, 12.91 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (4-((2-nitrophenyl)sulfonamido)pentan-2-yl)carbamate as a white solid (4.56 g, 11.77 mmol, quantitative). UPLC/ELSD: RT=0.78 min. MS (ES): m/z (MH⁺) 388.4 for C₁₆H₂₅N₃O₆S. ¹H NMR (300 MHz, CDCl₃) δ: ppm 8.10 (m, 1H), 7.79 (m, 1H), 7.66 (m, 2H), 5.31 (br. s, 1H), 4.29 (br. s, 1H), 3.59 (m, 2H), 1.64 (m, 2H), 1.38 (s, 9H), 1.05 (t, 6H).

### Step 2: di-tert-butyl ((butane-1,4-diylbis(azanediyl))bis(pentane-4,2-diyl))dicarbamate

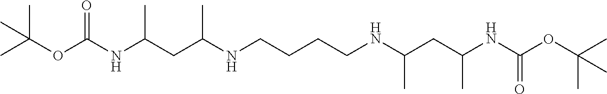 [SMILES: CC(CC(C)NC(=O)OC(C)(C)C)NCCCCNC(C)CC(C)NC(=O)OC(C)(C)C]

To a solution of tert-butyl (4-((2-nitrophenyl)sulfonamido)pentan-2-yl)carbamate (4.56 g, 11.77 mmol) in dry DMF (50 mL) set stirring under nitrogen was added potassium carbonate (4.72 g, 34.18 mmol) and 1,4-diiodobutane (0.74 mL, 5.60 mmol). The solution was heated to 40° C. and allowed to proceed overnight. The following morning, benzyl bromide (0.55 mL, 4.65 mmol) was added and the reaction was allowed to proceed at room temperature for 8 h. Then, thiophenol (2.21 mL, 21.57 mmol), potassium carbonate (2.32 g, 16.81 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(pentane-4,2-diyl))dicarbamate as a colorless oil (2.07 g, 4.51 mmol, 80.5%). UPLC/ELSD: RT=0.27 min. MS (ES): m/z (MH⁺) 459.6 for C₂₄H₅₀N₄O₄. ¹H NMR (300 MHz, CDCl₃) δ: ppm 5.31 (m, 2H), 3.77 (m, 2H), 2.75 (m, 4H), 2.51 (m, 2H), 1.56 (m, 7H), 1.45 (s, 20H), 1.17 (m, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate

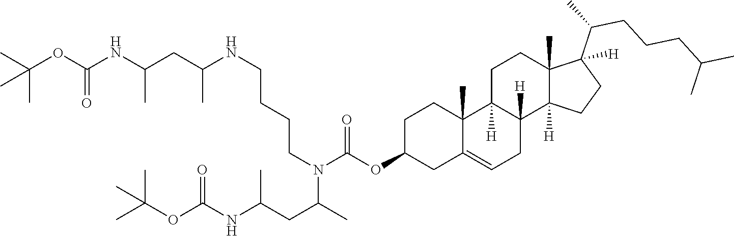

To a solution of di-tert-butyl ((butane-1,4-diylbis(azanediyl))bis(pentane-4,2-diyl))dicarbamate (0.83 g, 1.80 mmol) in dry toluene (20 mL) set stirring under nitrogen was added triethylamine (0.76 mL, 5.40 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.99 g, 1.80 mmol) was added, and the solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate as a colorless oil (0.70 g, 0.80 mmol, 44.4%). UPLC/ELSD: RT=2.74 min. MS (ES): m/z (MH⁺) 872.3 for C₅₂H₉₄N₄O₆. ¹H NMR (300 MHz, CDCl₃) δ: ppm 5.26 (m, 1H), 4.61 (m, 1H), 4.41 (br. m, 1H), 3.69 (br. m, 4H), 2.97 (m, 2H), 2.58 (m, 2H), 2.24 (br. m, 4H), 1.89 (m, 6H), 1.44 (m, 11H), 1.33 (s, 20H), 1.24 (br. m, 5H), 1.04 (m, 19H), 0.92 (s, 5H), 0.83 (d, 4H, J=6 Hz), 0.77 (d, 6H, J=6 Hz), 0.58 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride

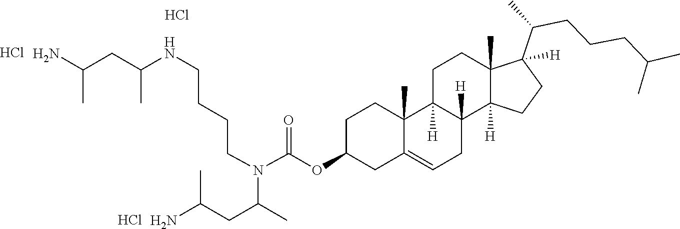

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-((tert-butoxycarbonyl)amino)pentan-2-yl)(4-((4-((tert-butoxycarbonyl)amino)pentan-2-yl)amino)butyl)carbamate (0.70 g, 0.80 mmol) in isopropanol (10 mL) set stirring under nitrogen was added hydrochloric acid (5 N in isopropanol, 1.60 mL, 7.99 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-aminopentan-2-yl)(4-((4-aminopentan-2-yl)amino)butyl)carbamate trihydrochloride as a white solid (0.46 g, 0.57 mmol, 70.7%). UPLC/ELSD: RT=1.96 min. MS (ES): m/z (MH⁺) 672.1 for C₄₂H₈₁Cl₃N₄O₂. ¹H NMR (300 MHz, MeOD) δ: ppm 5.42 (m, 1H), 4.47 (br. m, 1H), 4.28 (m, 1H), 3.53 (br. m, 2H), 3.53 (m, 2H), 3.33 (s, 2H), 3.15 (m, 4H), 2.40 (m, 2H), 1.93 (br. m, 18H), 1.42 (br. m, 12H), 1.28 (br. m, 4H), 1.16 (d, 8H, J=6 Hz), 1.08 (m, 6H), 0.98 (d, 4H, J=9 Hz), 0.89 (d, 6H, J=6 Hz), 0.74 (s, 3H).

### BB. Compound SA127: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride

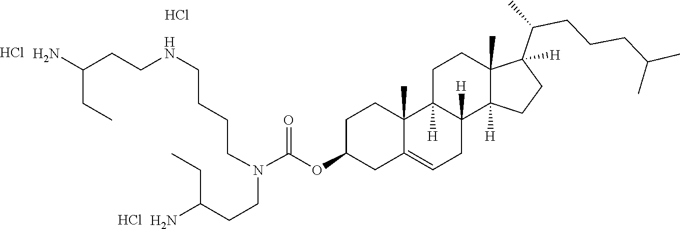

### Step 1: tert-butyl (1-((2-nitrophenyl)sulfonamido)pentan-3-yl)carbamate

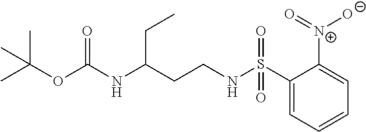 [SMILES: CCC(CCNS(=O)(=O)c1ccccc1[N+](=O)[O-])NC(=O)OC(C)(C)C]

To a solution of tert-butyl (1-aminopentan-3-yl)carbamate (2.00 g, 9.39 mmol) in dry DCM (50 mL) set stirring under nitrogen was added triethylamine (2.62 mL, 18.78 mmol). The solution was cooled to 0° C. and then a solution of 2-nitrobenzenesulfonyl chloride (2.29 g, 10.33 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (1-((2-nitrophenyl)sulfonamido)pentan-3-yl)carbamate as a white solid (3.73 g, 9.61 mmol, quantitative). UPLC/ELSD: RT=0.81 min. MS (ES): m/z (MH⁺) 388.4 for C₁₆H₂₅N₃O₆S. ¹H NMR (300 MHz, CDCl₃) δ: ppm 8.12 (m, 1H), 7.83 (m, 1H), 7.74 (m, 2H), 6.26 (br. s, 1H), 4.27 (br. s, 1H), 3.54 (m, 1H), 3.31 (m, 1H), 3.03 (m, 1H), 1.76 (m, 1H), 1.41 (s, 9H), 0.87 (t, 3H).

### Step 2: di-tert-butyl ((butane-1,4-diylbis(azanediyl))bis(pentane-1,3-diyl))dicarbamate

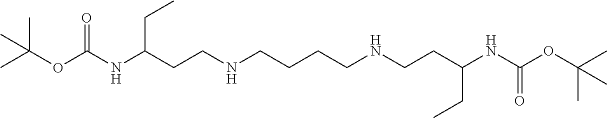 [SMILES: CCC(CCNCCCCNCCC(CC)NC(=O)OC(C)(C)C)NC(=O)OC(C)(C)C]

To a solution of tert-butyl (1-((2-nitrophenyl)sulfonamido)pentan-3-yl)carbamate (3.73 g, 9.61 mmol) in dry DMF (50 mL) set stirring under nitrogen was added potassium carbonate (3.86 g, 27.93 mmol) and 1,4-diiodobutane (0.60 mL, 4.58 mmol). The solution was heated to 40° C. and allowed to proceed overnight. The following morning, benzyl bromide (0.45 mL, 3.80 mmol) was added, and the reaction was allowed to proceed at room temperature for 8 h. Then thiophenol (1.80 mL, 17.63 mmol), potassium carbonate (1.90 g, 13.73 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(pentane-1,3-diyl))dicarbamate as a colorless oil (1.40 g, 3.05 mmol, 66.7%). UPLC/ELSD: RT=0.34 min. MS (ES): m/z (MH⁺) 459.6 for C₂₄H₅₀N₄O₄. ¹H NMR (300 MHz, CDCl₃) δ: ppm 4.94 (m, 2H), 3.29 (m, 4H), 2.47 (m, 8H), 1.53 (m, 2H), 1.36 (m, 8H), 1.21 (s, 19H), 0.69 (t, 6H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate

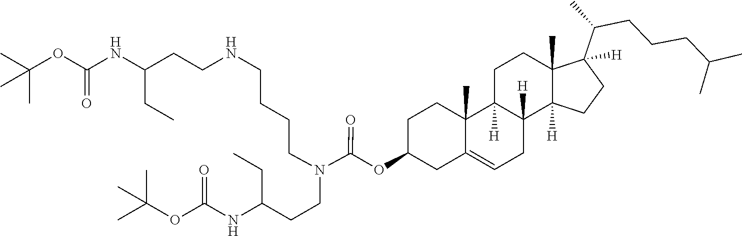

To a solution of di-tert-butyl ((butane-1,4-diylbis(azanediyl))bis(pentane-1,3-diyl))dicarbamate (0.58 g, 1.27 mmol) in dry toluene (20 mL) set stirring under nitrogen was added triethylamine (0.54 mL, 3.82 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.70 g, 1.27 mmol) was added, and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate as a colorless oil (0.67 g, 0.77 mmol, 60.5%). UPLC/ELSD: RT=3.06 min. MS (ES): m/z (MH⁺) 872.3 for C₅₂H₉₄N₄O₆. ¹H NMR (300 MHz, CDCl₃) δ: ppm 5.24 (m, 1H), 4.61 (br. m, 3H), 3.43 (br. m, 2H), 3.11 (br. m, 4H), 2.47 (m, 4H), 2.22 (m, 2H), 1.87 (br. m, 8H), 1.42 (m, 13H), 1.32 (s, 24H), 1.14 (br. m, 13H), 1.04 (m, 19H), 0.91 (s, 6H), 0.79 (d, 9H, J=6 Hz), 0.76 (d, 7H, J=6 Hz), 0.56 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride

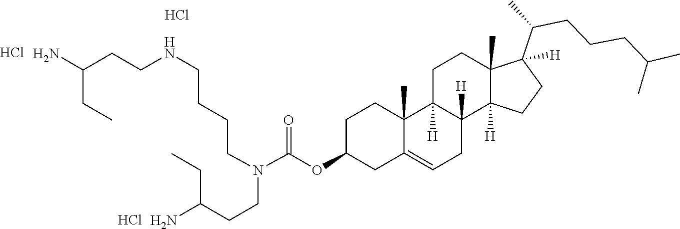

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)pentyl)(4-((3-((tert-butoxycarbonyl)amino)pentyl)amino)butyl)carbamate (0.67 g, 0.77 mmol) in isopropanol (10 mL) set stirring under nitrogen was added hydrochloric acid (5 N in isopropanol, 1.54 mL, 7.70 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminopentyl)(4-((3-aminopentyl)amino)butyl)carbamate trihydrochloride as a white solid (0.52 g, 0.65 mmol, 84.4%). UPLC/ELSD: RT=1.90 min. MS (ES): m/z (MH⁺) 672.1 for C₄₂H₈₁Cl₃N₄O₂. ¹H NMR (300 MHz, MeOD) δ: ppm 5.31 (m, 1H), 4.34 (br. m, 1H), 3.81 (m, 2H), 3.22 (br. m, 6H), 3.01 (m, 5H), 2.26 (m, 2H), 1.98 (m, 2H), 1.94 (s, 4H), 1.81 (br. m, 5H), 1.63 (br. m, 8H), 1.44 (br. m, 7H), 1.28 (br. m, 5H), 1.06 (d, 15H, J=9 Hz), 0.96 (m, 11H), 0.84 (d, 4H, J=6 Hz), 0.80 (d, 6H, J=6 Hz), 0.63 (s, 3H).

### BC. Compound SA128: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride

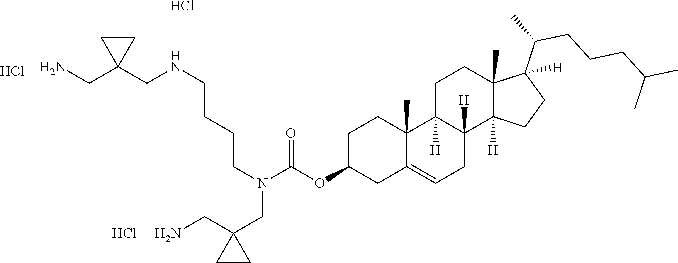

### Step 1: tert-butyl ((1-(((2-nitrophenyl)sulfonamido)methyl)cyclopropyl)methyl)carbamate

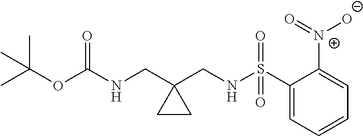 [SMILES: CC(C)(C)OC(=O)NCC1(CNS(=O)(=O)c2ccccc2[N+](=O)[O-])CC1]

To a solution of tert-butyl ((1-(aminomethyl)cyclopropyl)methyl)carbamate (2.50 g, 11.86 mmol) in dry DCM (25 mL) set stirring under nitrogen was added triethylamine (3.31 mL, 23.72 mmol). The solution was cooled to 0° C. and then a solution of 2-nitrobenzenesulfonyl chloride (2.89 g, 13.04 mmol) in 50 mL dry DCM was added dropwise over 30 minutes. The reaction was allowed to proceed at 0° C. for an hour, and then at room temperature for an additional three hours. Following, the mixture was diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl ((1-(((2-nitrophenyl)sulfonamido)methyl)cyclopropyl)methyl)carbamate as a white solid (4.60 g, 11.92 mmol, quantitative). UPLC/ELSD: RT=0.83 min. MS (ES): m/z (MH⁺) 386.4 for C₁₆H₂₃N₃O₆S. ¹H NMR (300 MHz, CDCl₃) δ: ppm 8.11 (m, 1H), 7.85 (m, 1H), 7.75 (m, 2H), 6.33 (br. s, 1H), 4.82 (br. s, 1H), 3.08 (d, 2H, J=6 Hz), 3.02 (d, 2H, J=6 Hz), 1.45 (s, 9H), 0.48 (m, 4H).

### Step 2: di-tert-butyl ((((butane-1,4-diylbis(azanediyl))bis(methylene))bis(cyclopropane-1,1-diyl))bis(methylene))dicarbamate

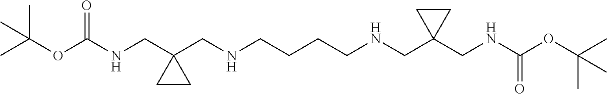 [SMILES: CC(C)(C)OC(=O)NCC1(CNCCCCNCC2(CNC(=O)OC(C)(C)C)CC2)CC1]

To a solution of tert-butyl ((1-(((2-nitrophenyl)sulfonamido)methyl)cyclopropyl)methyl)carbamate (4.60 g, 11.92 mmol) in dry DMF (50 mL) set stirring under nitrogen was added potassium carbonate (4.79 g, 34.64 mmol) and 1,4-diiodobutane (0.75 mL, 5.68 mmol). The solution was heated to 40° C. and allowed to proceed overnight. The following morning, benzyl bromide (0.56 mL, 4.71 mmol) was added, and the reaction was allowed to proceed at room temperature for 8 h. Then thiophenol (2.24 mL, 21.86 mmol), potassium carbonate (2.35 g, 17.03 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((((butane-1,4-diylbis(azanediyl))bis(methylene))bis(cyclopropane-1,1-diyl))bis(methylene))dicarbamate as a colorless oil (2.47 g, 5.43 mmol, 95.6%). UPLC/ELSD: RT=0.28 min. MS (ES): m/z (MH⁺) 455.6 for C₂₄H₄₆N₄O₄. ¹H NMR (300 MHz, CDCl₃) δ: ppm 5.67 (m, 2H), 2.87 (d, 4H, J=6 Hz), 2.41 (m, 4H), 2.34 (s, 4H), 1.36 (m, 5H), 1.28 (s, 19H), 0.25 (m, 4H), 0.17 (m, 4H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate

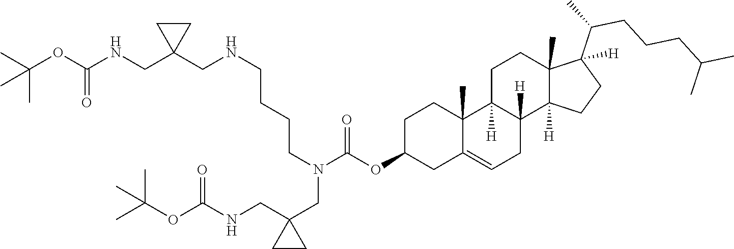

To a solution of di-tert-butyl ((((butane-1,4-diylbis(azanediyl))bis(methylene))bis(cyclopropane-1,1-diyl))bis(methylene))dicarbamate (0.92 g, 2.02 mmol) in dry toluene (20 mL) set stirring under nitrogen was added triethylamine (0.85 mL, 6.04 mmol). Then (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (1.11 g, 2.02 mmol) was added. The solution was heated to 90° C. and allowed to proceed overnight. The following morning, the reaction mixture was allowed to cool to room temperature, diluted with toluene, and washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate as a colorless oil (0.83 g, 0.95 mmol, 47.3%). UPLC/ELSD: RT=3.05 min. MS (ES): m/z (MH⁺) 868.3 for C₅₂H₉₀N₄O₆. ¹H NMR (300 MHz, CDCl₃) δ: ppm 5.77 (br. m, 1H), 5.29 (m, 1H), 4.44 (br. m, 1H), 3.13 (br. m, 4H), 2.97 (m, 2H), 2.85 (m, 2H), 2.50 (t, 2H), 2.42 (s, 2H), 2.26 (br. m, 2H), 1.85 (br. m, 5H), 1.47 (m, 9H), 1.34 (s, 19H), 1.05 (br. m, 11H), 0.94 (s, 6H), 0.84 (d, 4H, J=6 Hz), 0.78 (d, 6H, J=6 Hz), 0.59 (s, 3H), 0.50 (m, 2H), 0.35 (m, 2H), 0.26 (m, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride

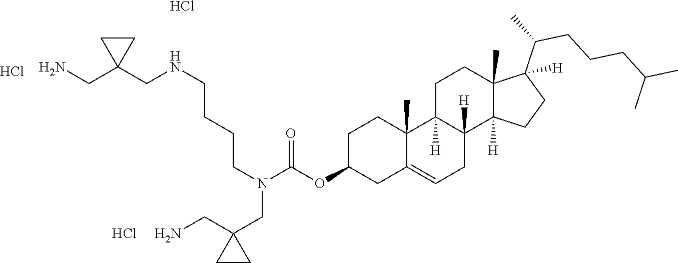

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(((tert-butoxycarbonyl)amino)methyl)cyclopropyl)methyl)(4-(((1-(((tert-butoxycarbonyl)amino)methyl)cyclopropyl)methyl)amino)butyl)carbamate (0.83 g, 0.95 mmol) in isopropanol (10 mL) set stirring under nitrogen was added hydrochloric acid (5 N in isopropanol, 1.91 mL, 9.54 mmol) dropwise. The solution was heated to 40° C. and allowed to proceed overnight. The following morning, the mixture was cooled to room temperature, and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl ((1-(aminomethyl)cyclopropyl)methyl)(4-(((1-(aminomethyl)cyclopropyl)methyl)amino)butyl)carbamate trihydrochloride as a white solid (0.67 g, 0.83 mmol, 86.8%). UPLC/ELSD: RT=1.61 min. MS (ES): m/z (MH⁺) 668.1 for C₄₂H₇₇Cl₃N₄O₂. ¹H NMR (300 MHz, MeOD) δ: ppm 5.44 (m, 1H), 4.54 (br. m, 1H), 3.86 (m, 2H), 3.33 (br. m, 6H), 3.15 (m, 6H), 2.81 (m, 2H), 2.44 (m, 2H), 1.73 (br. m, 11H), 1.55 (br. m, 6H), 1.39 (m, 5H), 1.18 (d, 17H, J=6 Hz), 1.09 (s, 6H), 0.98 (d, 7H, J=9 Hz), 0.90 (d, 9H, J=6 Hz), 0.74 (br. m, 7H).

### BD. Compound SA129: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-((S)-2,5-diaminopentanoyl)glycinate trihydrochloride

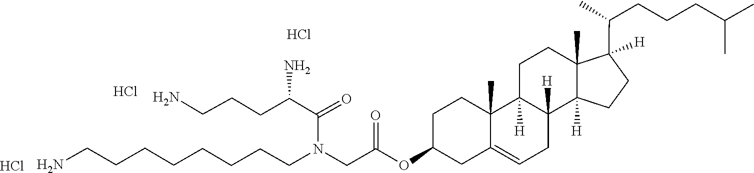

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-((S)-2,5-bis((tert-butoxycarbonyl)amino)pentanoyl)-N-(8-((tert-butoxycarbonyl)amino)octyl)glycinate

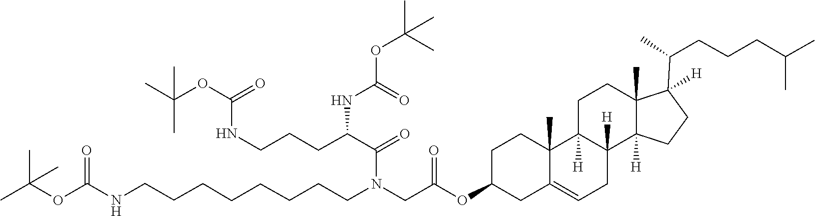

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.250 g, 0.373 mmol) and (2S)-2,5-bis[(tert-butoxycarbonyl)amino]pentanoic acid (0.161 g, 0.484 mmol) in DCM (3.75 mL) was cooled to 0° C. in an ice bath. Then 1-ethyl-3-(3-dimethylaminopropyl)carbodiimide hydrochloride (0.107 g, 0.559 mmol) was added. The reaction mixture stirred at rt and was monitored by LCMS. At 17 h, the reaction mixture was cooled to 0° C. in an ice bath, and then water (3.0 mL) was added. The layers were separated, and the aqueous layer was extracted with DCM (10 mL). The combined organics were washed with 5% aq. NaHCO₃ solution, passed through a hydrophobic frit, dried over Na₂SO₄, and concentrated. The crude material was purified via silica gel chromatography (10-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-((S)-2,5-bis((tert-butoxycarbonyl)amino)pentanoyl)-N-(8-((tert-butoxycarbonyl)amino)octyl)glycinate (0.380 g, quant.) as a clear oil. UPLC/ELSD: RT=4.03 min. MS (ES): m/z=987.54 [M+H]⁺ for C₅₇H₁₀₀N₄O₉. ¹H NMR (300 MHz, CDCl₃): δ 5.20-5.43 (m, 2H), 4.24-4.80 (m, 4H), 4.29 (d, 1H, J=17.0 Hz), 3.72 (d, 1H, J=17.2 Hz), 3.02-3.53 (m, 6H), 2.20-2.42 (m, 2H), 0.93-2.18 (br. m, 69H), 1.01 (s, 3H), 0.91 (d, 3H, J=6.5 Hz), 0.86 (d, 3H, J=6.6 Hz), 0.86 (d, 3H, J=6.6 Hz), 0.63-0.76 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-((S)-2,5-diaminopentanoyl)glycinate trihydrochloride

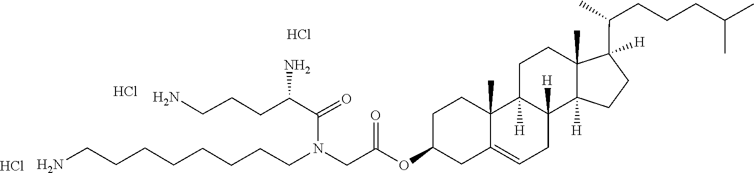

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-((S)-2,5-bis((tert-butoxycarbonyl)amino)pentanoyl)-N-(8-((tert-butoxycarbonyl)amino)octyl)glycinate (0.356 g, 0.347 mmol) in iPrOH (3.5 mL) was added 5-6 N HCl in iPrOH (0.50 mL). The reaction mixture stirred at 40° C. and was monitored by LCMS. At 19 h, additional 5-6 N HCl in iPrOH (0.10 mL) was added. At 22 h, the reaction mixture was cooled to rt, and then ACN (7 mL) was added. The suspension was cooled in an ice bath, and then solids were collected by vacuum filtration rinsing with 2:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(8-aminooctyl)-N-((S)-2,5-diaminopentanoyl)glycinate trihydrochloride (0.182 g, 0.203 mmol, 58.6%) as a white solid. UPLC/ELSD: RT=1.94 min. MS (ES): m/z=343.40 [M+2H]²⁺ for C₄₂H₇₆N₄O₃. ¹H NMR (300 MHz, DMSO): δ 7.85-8.59 (m, 9H), 5.24-5.55 (m, 1H), 4.25-4.68 (m, 2H), 4.17 (d, 1H, J=17.0 Hz), 3.95 (d, 1H, J=17.2 Hz), 3.12-3.61 (m, 2H), 2.65-2.85 (m, 4H), 2.20-2.42 (m, 2H), 0.92-2.03 (br. m, 42H), 0.98 (s, 3H), 0.89 (d, 3H, J=6.3 Hz), 0.84 (d, 3H, J=6.6 Hz), 0.84 (d, 3H, J=6.5 Hz), 0.65 (s, 3H).

### BE. Compound SA130: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride

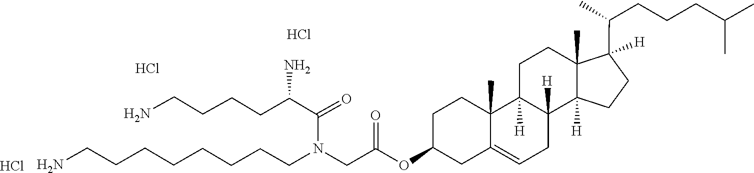

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(N₂,N₆-bis(tert-butoxycarbonyl)-L-lysyl)-N-(8-((tert-butoxycarbonyl)amino)octyl)glycinate

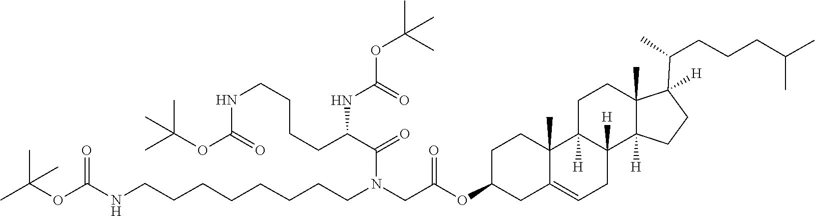

A solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (8-((tert-butoxycarbonyl)amino)octyl)glycinate (0.235 g, 0.350 mmol) and (2S)-2,6-bis[(tert-butoxycarbonyl)amino]hexanoic acid (0.121 g, 0.350 mmol) in DCM (3.5 mL) was cooled to 0° C. in an ice bath. Then 1-ethyl-3-(3-dimethylaminopropyl)carbodiimide hydrochloride (0.101 g, 0.525 mmol) was added. The reaction mixture stirred at rt and was monitored by LCMS. At 19 h, the reaction mixture was cooled to 0° C., and then additional (2S)-2,6-bis[(tert-butoxycarbonyl)amino]hexanoic acid (23 mg) and 1-ethyl-3-(3-dimethylaminopropyl)carbodiimide hydrochloride (20 mg) were added. The reaction mixture stirred at rt. At 21 h, the reaction mixture was cooled to 0° C. in an ice bath, and then water (3.5 mL) was added. The layers were separated, and the aqueous layer was extracted with DCM (10 mL). The combined organics were washed with 5% aq. NaHCO₃ solution, passed through a hydrophobic frit, dried over Na₂SO₄, and concentrated. The crude material was purified via silica gel chromatography (10-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(N2,N6-bis(tert-butoxycarbonyl)-L-lysyl)-N-(8-((tert-butoxycarbonyl)amino)octyl)glycinate (0.323 g, 0.323 mmol, 92.3%) as a clear oil. UPLC/ELSD: RT=4.09 min. MS (ES): m/z=1001.35 [M+H]⁺ for C₅₈H₁₀₂N₄O₉. ¹H NMR (300 MHz, CDCl₃): δ 5.21-5.46 (m, 2H), 4.26-4.79 (m, 4H), 4.30 (d, 1H, J=17.1 Hz), 3.71 (d, 1H, J=17.0 Hz), 3.01-3.52 (m, 6H), 2.22-2.43 (m, 2H), 0.93-2.14 (br. m, 71H), 1.01 (s, 3H), 0.91 (d, 3H, J=6.4 Hz), 0.86 (d, 6H, J=6.6 Hz), 0.64-0.75 (m, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride

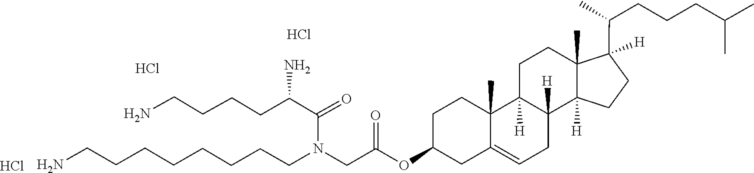

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(N2,N6-bis(tert-butoxycarbonyl)-L-lysyl)-N-(8-((tert-butoxycarbonyl)amino)octyl)glycinate (0.303 g, 0.303 mmol) in iPrOH (3.0 mL) was added 5-6 N HCl in iPrOH (0.45 mL). The reaction mixture stirred at 40° C. and was monitored by LCMS. At 19 h, additional 5-6 N HCl in iPrOH (0.10 mL) was added. At 22 h, the reaction mixture was cooled to rt, and then ACN (6 mL) was added. The suspension was cooled to 0° C. in an ice bath, and then solids were collected by vacuum filtration rinsing with 2:1 ACN/iPrOH to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N-(L-lysyl)-N-(8-aminooctyl)glycinate trihydrochloride (0.163 g, 0.173 mmol, 57.0%) as a white solid. UPLC/ELSD: RT=1.96 min. MS (ES): m/z=350.43 [M+2H]²¹ for C₄₃H₇₈N₄O₃. ¹H NMR (300 MHz, DMSO): δ 7.89-8.51 (m, 9H), 5.22-5.41 (m, 1H), 4.23-4.61 (m, 2H), 4.19 (d, 1H, J=17.0 Hz), 3.93 (d, 1H, J=17.1 Hz), 3.13-3.51 (m, 2H), 2.64-2.85 (m, 4H), 2.22-2.36 (m, 2H), 0.92-2.04 (br. m, 44H), 0.98 (s, 3H), 0.90 (d, 3H, J=6.3 Hz), 0.84 (d, 6H, J=6.6 Hz), 0.65 (s, 3H).

### BF. Compound SA131: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)-L-lysinate trihydrochloride

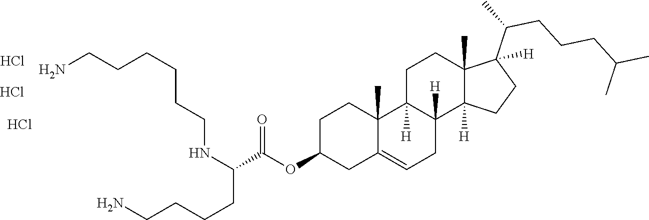

### Step 1: Methyl N6-(tert-butoxycarbonyl)-N2-((2-nitrophenyl)sulfonyl)-L-lysinate

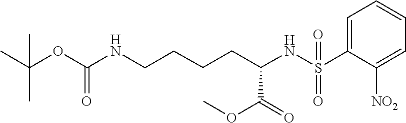 [SMILES: COC(=O)[C@H](CCCCNC(=O)OC(C)(C)C)NS(=O)(=O)c1ccccc1[N+](=O)[O-]]

A mixture of methyl (2S)-2-amino-6-[(tert-butoxycarbonyl)amino]hexanoate hydrochloride (1.000 g, 3.369 mmol), DMAP (cat.), and triethylamine (1.40 mL, 9.96 mmol) in DCM (15 mL) was cooled to 0° C., and then 2-nitrobenzenesulfonyl chloride (0.896 g, 4.04 mmol) in DCM (5 mL) was added dropwise. The reaction mixture stirred at rt and was monitored by LCMS. At 1 h, the reaction mixture was cooled to 0° C., and then water (20 mL) was added. The layers were separated, and the organic layer was washed with 5% aq. NaHCO₃ solution, dried over Na₂SO₄, and concentrated. The crude material was purified via silica gel chromatography (30-70% EtOAc in hexanes) to afford methyl N6-(tert-butoxycarbonyl)-N₂-((2-nitrophenyl)sulfonyl)-L-lysinate (1.44 g, 3.23 mmol, 95.9%) as a viscous, yellow oil. UPLC/ELSD: RT=0.78 min. MS (ES): m/z=390.30 [(M+H)—((CH₃)₂C═CH₂)]⁺ for C₁₈H₂₇N₃O₈S. ¹H NMR (300 MHz, CDCl₃): δ 8.02-8.10 (m, 1H), 7.89-7.97 (m, 1H), 7.68-7.78 (m, 2H), 6.08 (d, 1H, J=9.1 Hz), 4.53 (br. s, 1H), 4.16 (td, 1H, J=8.5, 5.0 Hz), 3.47 (s, 3H), 3.09 (td, 2H, J=6.1, 6.1 Hz), 1.33-1.94 (m, 6H), 1.44 (s, 9H).

### Step 2: Methyl N6-(tert-butoxycarbonyl)-N2-(6-((tert-butoxycarbonyl)amino)hexyl)-N2-((2-nitrophenyl)sulfonyl)-L-lysinate

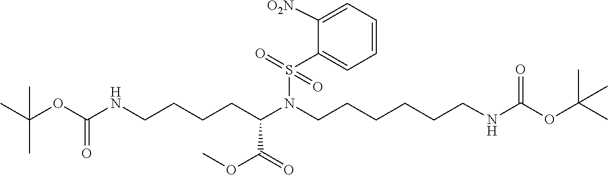 [SMILES: COC(=O)[C@H](CCCCNC(=O)OC(C)(C)C)N(CCCCCCNC(=O)OC(C)(C)C)S(=O)(=O)c1ccccc1[N+](=O)[O-]]

Methyl (2S)-6-[(tert-butoxycarbonyl)amino]-2-(2-nitrobenzenesulfonamido)hexanoate (0.603 g, 1.35 mmol), tert-butyl N-(6-bromohexyl)carbamate (0.504 g, 1.80 mmol), potassium carbonate (0.480 g, 3.48 mmol), and potassium iodide (0.046 g, 0.28 mmol) were combined in DMF (9.0 mL) and stirred at 80° C. Reaction was monitored by LCMS. At 18 h, the reaction mixture was cooled to rt, filtered, diluted with MTBE (100 mL), washed with water (3×) and brine, dried over Na₂SO₄, and concentrated. The crude material was purified via silica gel chromatography (20-70% EtOAc in hexanes) to afford methyl N6-(tert-butoxycarbonyl)-N2-(6-((tert-butoxycarbonyl)amino)hexyl)-N2-((2-nitrophenyl)sulfonyl)-L-lysinate (0.693 g, 1.08 mmol, 79.4%) as a clear oil. UPLC/ELSD: RT=1.53 min. MS (ES): m/z=489.29 [(M+H)-2((CH₃)₂C═CH₂)—CO₂]⁺ for C₂₉H₄₈N₄O₁₀S. ¹H NMR (300 MHz, CDCl₃): δ 7.99-8.09 (m, 1H), 7.64-7.75 (m, 2H), 7.52-7.61 (m, 1H), 4.42-4.73 (m, 3H), 3.54 (s, 3H), 3.31-3.45 (m, 1H), 2.99-3.21 (m, 5H), 1.94-2.13 (m, 1H), 1.20-1.89 (br. m, 13H), 1.44 (s, 18H).

### Step 3: N6-(tert-butoxycarbonyl)-N2-(6-((tert-butoxycarbonyl)amino)hexyl)-N2-((2-nitrophenyl)sulfonyl)-L-lysine

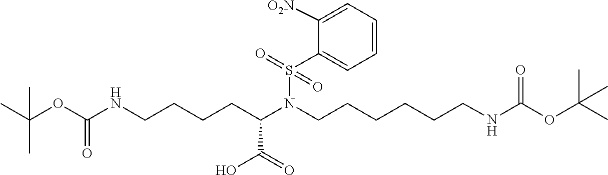 [SMILES: CC(C)(C)OC(=O)NCCCCCCN([C@@H](CCCCNC(=O)OC(C)(C)C)C(=O)O)S(=O)(=O)c1ccccc1[N+](=O)[O-]]

To a solution of methyl (2S)-6-[(tert-butoxycarbonyl)amino]-2-(N-{6-[(tert-butoxycarbonyl)amino]hexyl}-2-nitrobenzenesulfonamido)hexanoate (0.690 g, 1.07 mmol) in THE (7.0 mL) and MeOH (1.4 mL) was added aq. lithium hydroxide monohydrate (0.90 mL, 15 w/v %). The reaction mixture stirred at rt and was monitored by LCMS. At 19 h, the reaction mixture was concentrated to remove volatile organics, and then partitioned between water (50 mL) and EtOAc (50 mL). The biphasic mixture was washed with 5% aq. K₂CO₃ and 0.1 N aq. HCl, dried over Na₂SO₄, and concentrated to afford (2S)-6-[(tert-butoxycarbonyl)amino]-2-(N-{6-[(tert-butoxycarbonyl)amino]hexyl}-2-nitrobenzenesulfonamido)hexanoic acid (0.578 g, 0.916 mmol, 85.6%) as an amber oil. UPLC/ELSD: RT=1.27 min. MS (ES): m/z=475.35 [(M+H)-2((CH₃)₂C═CH₂)—CO₂]⁺ for C₂₈H₄₆N₄O₁₀S. ¹H NMR (300 MHz, CDCl₃): δ 8.03-8.12 (m, 1H), 7.63-7.74 (m, 2H), 7.53-7.62 (m, 1H), 4.48-4.79 (m, 3H), 2.94-3.42 (m, 6H), 1.92-2.18 (m, 1H), 1.20-1.83 (br. m, 13H), 1.44 (s, 18H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-N2-((2-nitrophenyl)sulfonyl)-L-lysinate

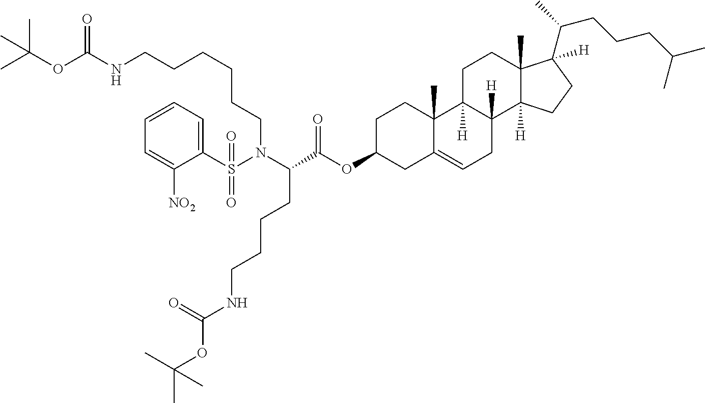

A mixture of (2S)-6-[(tert-butoxycarbonyl)amino]-2-(N-{6-[(tert-butoxycarbonyl)amino]hexyl}-2-nitrobenzenesulfonamido)hexanoic acid (0.560 g, 0.888 mmol), cholesterol (0.378 g, 0.977 mmol), and DMAP (cat.) in DCM (8.5 mL) was cooled to 0° C. Then 1-ethyl-3-(3-dimethylaminopropyl)carbodiimide hydrochloride (0.238 g, 1.24 mmol) was added. The reaction mixture slowly came to rt while stirring and was monitored by LCMS. At 19 h, the reaction mixture was cooled to 0° C., and then 5% aq. NaHCO₃ solution (8.5 mL) was added. Once the reaction mixture warmed to rt, the layers were separated. The aqueous layer was extracted with DCM (8 mL). The combined organics were passed through a hydrophobic frit, dried over Na₂SO₄, and concentrated. The crude material was purified via silica gel chromatography (0-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-N2-((2-nitrophenyl)sulfonyl)-L-lysinate (0.562 g, 0.562 mmol, 63.3%) as a white foam. UPLC/ELSD: RT=3.62 min. MS (ES): m/z=900.19 [(M+H)—((CH₃)₂C═CH₂)—CO₂]⁺ for C₅₅H₉₀N₄O₁₀S. ¹H NMR (300 MHz, CDCl₃): δ 7.99-8.10 (m, 1H), 7.64-7.74 (m, 2H), 7.52-7.60 (m, 1H), 5.24-5.36 (m, 1H), 4.36-4.81 (m, 4H), 3.33-3.51 (m, 1H), 2.94-3.24 (m, 5H), 1.48 (br. m, 60H), 0.93 (s, 3H), 0.90 (d, 3H, J=6.4 Hz), 0.86 (d, 3H, J=6.6 Hz), 0.86 (d, 3H, J=6.6 Hz), 0.66 (s, 3H).

### Step 5: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-L-lysinate

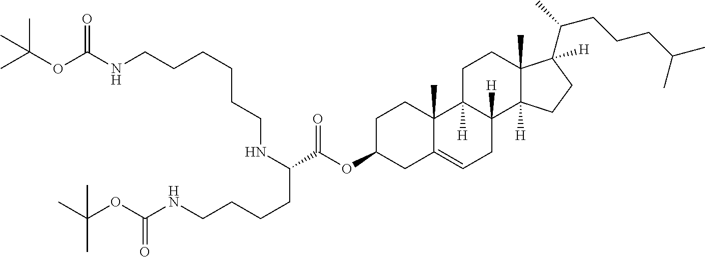

To a mixture of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-N2-((2-nitrophenyl)sulfonyl)-L-lysinate (0.540 g, 0.540 mmol) and potassium carbonate (0.224 g, 1.621 mmol) in DMF (6.5 mL) was added thiophenol (0.10 mL, 0.980 mmol). The reaction mixture stirred at rt and was monitored by LCMS. At 17 h, the LCMS data was consistent with reaction completion. The reaction mixture was diluted with DCM (20 mL), and then filtered through a pad of Celite®. The filtrate was diluted to 80 mL with DCM, and then washed with water (3×) and 5% aq. NaHCO₃ solution. The organics were passed through a hydrophobic frit, dried over Na₂SO₄, and concentrated. The crude material was purified via silica gel chromatography (40-80% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-L-lysinate (0.400 g, 0.491 mmol, 90.9%) as a clear oil. UPLC/ELSD: RT=2.92 min. MS (ES): m/z=815.18 [M+H]⁺ for C₄₉H₈₇N₃O₆. ¹H NMR (300 MHz, CDCl₃): δ 5.34-5.43 (m, 1H), 4.30-4.75 (m, 3H), 3.20-3.32 (m, 1H), 3.00-3.20 (m, 4H), 2.47-2.71 (m, 2H), 2.22-2.43 (m, 2H), 0.93-2.13 (br. m, 58H), 1.02 (s, 3H), 0.91 (d, 3H, J=6.4 Hz), 0.86 (d, 3H, J=6.6 Hz), 0.86 (d, 3H, J=6.6 Hz), 0.68 (s, 3H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)-L-lysinate trihydrochloride

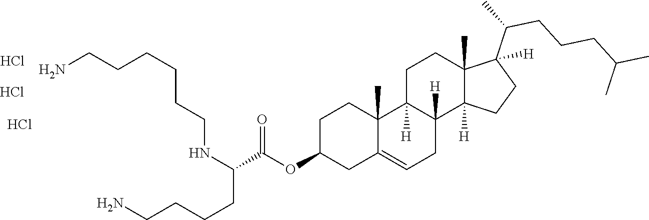

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl N6-(tert-butoxycarbonyl)-N2-(6-((tert-butoxycarbonyl)amino)hexyl)-L-lysinate (0.379 g, 0.465 mmol) in iPrOH (5.5 mL) was added 5-6 N HCl in iPrOH (0.93 mL). The reaction mixture stirred at 40° C. and was monitored by LCMS. At 16 h, the reaction mixture was cooled to rt, and then iPrOH (30 mL) was added. The suspension was centrifuged (10,000×g for 30 min). The supernatant was decanted, and then solids were suspended in MTBE (35 mL). The suspension was centrifuged (10,000×g for 30 min). The supernatant was decanted, and the solids were suspended in heptanes and then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (6-aminohexyl)-L-lysinate trihydrochloride (0.133 g, 0.166 mmol, 35.7%) as a white solid. UPLC/ELSD: RT=1.85 min. MS (ES): m/z=328.58 [(M+2H)+CH₃CN]²⁺ for C₃₉H₇₁N₃O₂. ¹H NMR (300 MHz, DMSO): δ 9.85 (br. s, 1H), 9.33 (br. s, 1H), 7.71-8.43 (m, 6H), 5.26-5.52 (m, 1H), 4.50-4.76 (m, 1H), 3.87-4.07 (m, 1H), 2.67-3.06 (m, 6H), 2.25-2.44 (m, 2H), 0.92-2.11 (br. m, 40H), 0.99 (s, 3H), 0.89 (d, 3H, J=6.3 Hz), 0.84 (d, 6H, J=6.5 Hz), 0.65 (s, 3H).

### BG. Compound SA132: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-amino-2-((6-aminohexyl)amino)pentanoate trihydrochloride

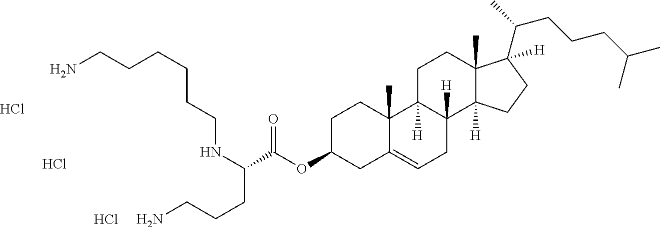

### Step 1: Methyl (2S)-5-[(tert-butoxycarbonyl)amino]-2-(2-nitrobenzenesulfonamido)pentanoate

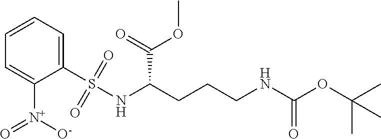 [SMILES: COC(=O)[C@H](CCCNC(=O)OC(C)(C)C)NS(=O)(=O)c1ccccc1[N+](=O)[O-]]

A solution of methyl (S)-2-amino-5-((tert-butoxycarbonyl)amino)pentanoate hydrochloride (1.000 g, 3.537 mmol) and triethylamine (1.50 mL, 10.7 mmol) in DCM (15 mL) was cooled to 0° C. in an ice bath, and then 2-nitrobenzenesulfonyl chloride (0.940 g, 4.24 mmol) in DCM (5.0 mL) was added dropwise. The reaction mixture stirred at rt and was monitored by LCMS. At 17 h, the reaction mixture was cooled to 0° C. in an ice bath, and then water (20 mL) was added. The layers were separated, and the organics were washed with 5% aq. NaHCO₃ solution, passed through a hydrophobic frit, dried over Na₂SO₄, and concentrated. The crude material was purified via silica gel chromatography (30-70% EtOAc in hexanes) to afford methyl (2S)-5-[(tert-butoxycarbonyl)amino]-2-(2-nitrobenzenesulfonamido)pentanoate (1.366 g, 3.166 mmol, 89.5%) as a viscous, yellow oil. UPLC/ELSD: RT=0.68 min. MS (ES): m/z=376.23 [(M+H)—(CH₃)₂C═CH₂]⁺ for C₁₇H₂₅N₃O₈S. ¹H NMR (300 MHz, CDCl₃): δ 8.03-8.10 (m, 1H), 7.88-7.96 (m, 1H), 7.68-7.78 (m, 2H), 6.14 (d, 1H, J=9.0 Hz), 4.55 (br. s, 1H), 4.18 (td, 1H, J=8.4, 5.2 Hz), 3.47 (s, 3H), 3.14 (dt, 2H, J=6.0, 5.7 Hz), 1.82-1.97 (m, 1H), 1.55-1.80 (m, 3H), 1.44 (s, 9H).

### Step 2: Methyl (2S)-5-[(tert-butoxycarbonyl)amino]-2-(N-{6-[(tert-butoxycarbonyl)amino]hexyl}-2-nitrobenzenesulfonamido)pentanoate

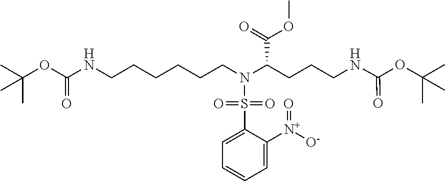 [SMILES: COC(=O)[C@H](CCCNC(=O)OC(C)(C)C)N(CCCCCCNC(=O)OC(C)(C)C)S(=O)(=O)c1ccccc1[N+](=O)[O-]]

Methyl (2S)-5-[(tert-butoxycarbonyl)amino]-2-(2-nitrobenzenesulfonamido)pentanoate (0.600 g, 1.39 mmol), tert-butyl N-(6-bromohexyl)carbamate (0.506 g, 1.81 mmol), potassium carbonate (0.480 g, 3.48 mmol), and potassium iodide (0.046 g, 0.28 mmol) were combined in DMF (9.0 mL). The reaction mixture stirred at 80° C. and was monitored by LCMS. At 2.5 h, the reaction mixture was cooled to rt and then filtered rinsing with MTBE. The filtrate was diluted with MTBE to 80 mL, washed with water (3×) and brine, dried over Na₂SO₄, and concentrated. The crude material was purified via silica gel chromatography (30-70% EtOAc in hexanes) to afford methyl (2S)-5-[(tert-butoxycarbonyl)amino]-2-(N-{6-[(tert-butoxycarbonyl)amino]hexyl}-2-nitrobenzenesulfonamido)pentanoate (0.720 g, 1.141 mmol, 82.1%) as a clear oil. UPLC/ELSD: RT=1.41 min. MS (ES): m/z=475.47 [(M+H)−2((CH₃)₂C═CH₂)—CO₂]⁺ for C₂₈H₄₆N₄O₁₀S. ¹H NMR (300 MHz, CDCl₃): δ 7.99-8.09 (m, 1H), 7.64-7.73 (m, 2H), 7.53-7.61 (m, 1H), 4.42-4.76 (m, 3H), 3.54 (s, 3H), 3.32-3.45 (m, 1H), 2.99-3.23 (m, 5H), 1.99-2.16 (m, 1H), 1.20-1.91 (br. m, 11H), 1.44 (s, 18H).

### Step 3: (2S)-5-[(tert-butoxycarbonyl)amino]-2-(N-{6-[(tert-butoxycarbonyl)amino]hexyl}-2-nitrobenzenesulfonamido)pentanoic acid

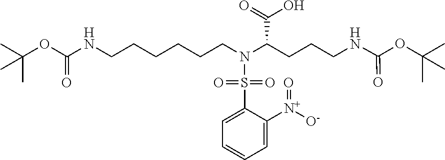 [SMILES: CC(C)(C)OC(=O)NCCCCCCN([C@@H](CCCNC(=O)OC(C)(C)C)C(=O)O)S(=O)(=O)c1ccccc1[N+](=O)[O-]]

To a solution of methyl (2S)-5-[(tert-butoxycarbonyl)amino]-2-(N-{6-[(tert-butoxycarbonyl)amino]hexyl}-2-nitrobenzenesulfonamido)pentanoate (0.716 g, 1.14 mmol) in THE (7.7 mL) and MeOH (1.5 mL) was added aq. lithium hydroxide monohydrate (0.96 mL, 15 w/v %). The reaction mixture stirred at rt and was monitored by LCMS. At 19 h, the reaction mixture was concentrated to remove volatile organics, taken up in water (50 mL), and extracted with EtOAc (3×25 mL). The combined organics were washed with a 5% aq. K₂CO₃ solution and then a 5% aq. citric acid solution, dried over Na₂SO₄, and concentrated to afford (2S)-5-[(tert-butoxycarbonyl)amino]-2-(N-{6-[(tert-butoxycarbonyl)amino]hexyl}-2-nitrobenzenesulfonamido)pentanoic acid (0.619 g, 1.00 mmol, 88.4%) as an amber oil. UPLC/ELSD: RT=1.22 min. MS (ES): m/z=461.4 [(M+H)−2((CH₃)₂C═CH₂)—CO₂]⁺ for C₂₇H₄₄N₄O₁₀S. ¹H NMR (300 MHz, CDCl₃): δ 8.02-8.11 (m, 1H), 7.62-7.72 (m, 2H), 7.54-7.62 (m, 1H), 4.47-4.84 (m, 3H), 2.98-3.44 (m, 6H), 1.94-2.13 (m, 1H), 1.20-1.83 (br. m, 11H), 1.44 (s, 18H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate

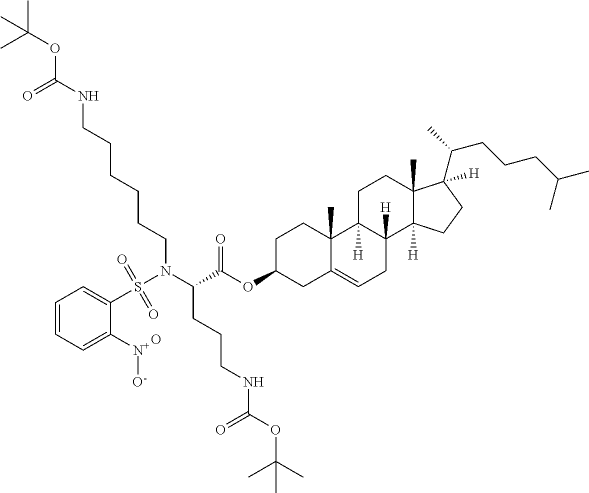

A mixture of (2S)-5-[(tert-butoxycarbonyl)amino]-2-(N-{6-[(tert-butoxycarbonyl)amino]hexyl}-2-nitrobenzenesulfonamido)pentanoic acid (0.520 g, 0.843 mmol), cholesterol (0.359 g, 0.927 mmol), and DMAP (cat.) in DCM (8.0 mL) was cooled to 0° C. Then 1-ethyl-3-(3-dimethylaminopropyl)carbodiimide hydrochloride (0.226 g, 1.18 mmol) was added. The reaction mixture slowly came to rt while stirring and was monitored by LCMS. At 22 h, the reaction mixture was cooled to 0° C., and then DMAP (cat.) and 1-ethyl-3-(3-dimethylaminopropyl)carbodiimide hydrochloride (105 mg) were added. The reaction mixture stirred at rt. At 26 h, the reaction mixture was cooled to 0° C. in an ice bath, and then water (8 mL) was added. The biphasic mixture came to rt and then was separated. The organics were washed with 5% aq. NaHCO₃ solution, passed through a hydrophobic frit, dried over Na₂SO₄, and concentrated. The crude material was purified via silica gel chromatography (0-40% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate (0.299 g, 0.303 mmol, 36.0%) as an amber foam. UPLC/ELSD: RT=3.59 min. MS (ES): m/z=829.74 [(M+H)-2((CH₃)₂C═CH₂)—CO₂]⁺ for C₅₄H₈₈N₄O₁₀S. ¹H NMR (300 MHz, CDCl₃): δ 8.00-8.09 (m, 1H), 7.63-7.74 (m, 2H), 7.53-7.60 (m, 1H), 5.23-5.35 (m, 1H), 4.35-4.84 (m, 4H), 3.33-3.53 (m, 1H), 2.93-3.27 (m, 5H), 0.94-2.17 (br. m, 58H), 0.92 (s, 3H), 0.90 (d, 3H, J=6.5 Hz), 0.86 (d, 3H, J=6.6 Hz), 0.86 (d, 3H, J=6.6 Hz), 0.66 (s, 3H).

### Step 5: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate

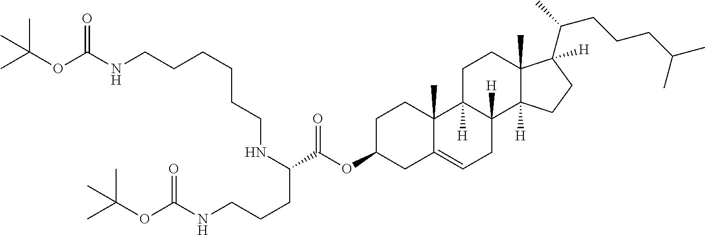

To a mixture of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((N-(6-((tert-butoxycarbonyl)amino)hexyl)-2-nitrophenyl)sulfonamido)pentanoate (0.284 g, 0.288 mmol) and potassium carbonate (0.119 g, 0.865 mmol) in DMF (5.0 mL) was added thiophenol (0.05 mL, 0.49 mmol). The reaction mixture stirred at rt and was monitored by LCMS. At 3 h, DCM (10 mL) was added, and the reaction mixture was filtered through a pad of Celite®. The filtrate was diluted with DCM to 80 mL and then was washed once with a 5% aq. NaHCO₃ solution and three times with water. The organics were passed through a hydrophobic frit, dried over Na₂SO₄, and concentrated. The crude material was purified via silica gel chromatography (40-80% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate (0.203 g, 0.254 mmol, 88.0%) as a clear, viscous oil. UPLC/ELSD: RT=2.92 min. MS (ES): m/z=801.37 [M+H]⁺ for C₄₈H₈₅N₃O₆. ¹H NMR (300 MHz, CDCl₃): δ 5.34-5.42 (m, 1H), 5.03-5.13 (m, 1H), 4.45-4.81 (m, 2H), 3.50-3.81 (m, 1H), 2.82-3.32 (m, 6H), 2.22-2.48 (m, 2H), 0.94-2.19 (br. m, 56H), 1.02 (s, 3H), 0.91 (d, 3H, J=6.4 Hz), 0.87 (d, 3H, J=6.6 Hz), 0.86 (d, 3H, J=6.6 Hz), 0.68 (s, 3H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-amino-2-((6-aminohexyl)amino)pentanoate trihydrochloride

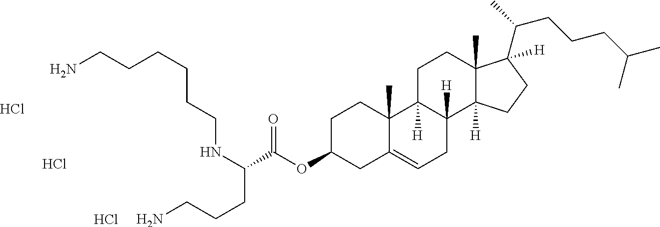

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-((tert-butoxycarbonyl)amino)-2-((6-((tert-butoxycarbonyl)amino)hexyl)amino)pentanoate (0.197 g, 0.246 mmol) in iPrOH (3.0 mL) was added 5-6 N HCl in iPrOH (0.49 mL). The reaction mixture stirred at 40° C. and was monitored by LCMS. At 16 h, the reaction mixture was cooled to rt, and then ACN (9 mL) was added. The suspension was filtered, but particles passed through frit.

The suspension was concentrated, and the residue suspended in MTBE (30 mL). The suspension was centrifuged (10,000×g for 30 min), and then the supernatant was decanted. Solids were suspended in heptanes, and then concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (S)-5-amino-2-((6-aminohexyl)amino)pentanoate trihydrochloride (0.147 g, 0.170 mmol, 69.0%) as a white solid. UPLC/ELSD: RT=1.86 min. MS (ES): m/z=321.42 [(M+2H)+CH₃CN]²⁺ for C₃₈H₆₉N₃O₂. ¹H NMR (300 MHz, DMSO): δ 9.75 (br. s, 1H), 9.40 (br. s, 1H), 7.61-8.32 (m, 6H), 5.31-5.48 (m, 1H), 4.53-4.72 (m, 1H), 3.92-4.16 (m, 1H), 2.66-3.05 (m, 6H), 2.24-2.45 (m, 2H), 0.92-2.13 (br. m, 38H), 1.00 (s, 3H), 0.90 (d, 3H, J=6.3 Hz), 0.84 (d, 3H, J=6.6 Hz), 0.84 (d, 3H, J=6.6 Hz), 0.66 (s, 3H).

### BH. Compound SA133: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

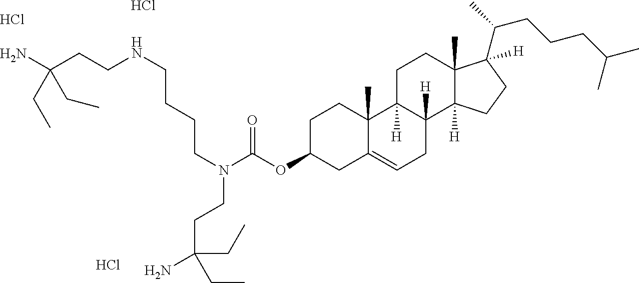

### Step 1: tert-butyl (3-ethyl-1-((2-nitrophenyl)sulfonamido)pentan-3-yl)carbamate

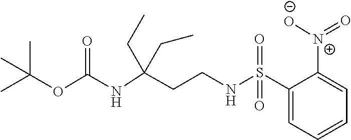 [SMILES: CCC(CC)(CCNS(=O)(=O)c1ccccc1[N+](=O)[O-])NC(=O)OC(C)(C)C]

To a solution of tert-butyl N-(1-amino-3-ethylpentan-3-yl)carbamate (2.50 g, 10.31 mmol) in dry DCM (50 mL) set stirring under nitrogen was added triethylamine (2.87 mL, 20.62 mmol). The solution was cooled to 0° C. and then a solution of 2-nitrobenzenesulfonyl chloride (2.51 g, 11.34 mmol) in 50 mL dry DCM was added dropwise over 30 minutes. The reaction was allowed to proceed at 0° C. for an hour and then at room temperature for an additional three hours. The mixture was then diluted with an additional 10 mL DCM, washed with saturated aqueous sodium bicarbonate (1×100 mL), water (1×100 mL), 10% aqueous citric acid (1×100 mL), water (1×100 mL), and brine (1×100 mL), dried over sodium sulfate, filtered, and concentrated to give tert-butyl (3-ethyl-1-((2-nitrophenyl)sulfonamido)pentan-3-yl)carbamate as a white solid (4.48 g, 10.31 mmol, quantitative). UPLC/ELSD: RT=1.27 min. MS (ES): m/z (MH⁺) 415.5 for C₁₈H₂₉N₃O₆S. ¹H NMR (300 MHz, CDCl₃) δ: ppm 8.12 (m, 1H), 7.85 (m, 1H), 7.76 (m, 2H), 5.41 (br. s, 1H), 4.19 (br. s, 1H), 3.14 (m, 2H), 1.92 (t, 2H), 1.63 (m, 2H), 1.45 (m, 2H), 1.40 (s, 9H), 0.78 (t, 6H).

### Step 2: di-tert-butyl ((butane-1,4-diylbis(azanediyl))bis(3-ethylpentane-1,3-diyl))dicarbamate

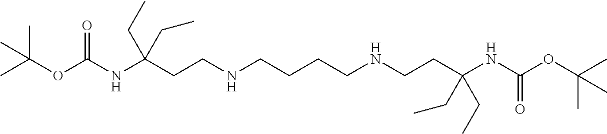 [SMILES: CCC(CC)(CCNCCCCNCCC(CC)(CC)NC(=O)OC(C)(C)C)NC(=O)OC(C)(C)C]

To a solution of tert-butyl (3-ethyl-1-((2-nitrophenyl)sulfonamido)pentan-3-yl)carbamate (4.48 g, 10.78 mmol) in dry DMF (50 mL) set stirring under nitrogen was added potassium carbonate (4.33 g, 31.32 mmol) and 1,4-diiodobutane (0.68 mL, 5.13 mmol). The solution was heated to 40° C. and allowed to proceed overnight. The following morning, benzyl bromide (0.51 mL, 4.26 mmol) was added and the reaction was allowed to proceed at room temperature for 8 h. Then, thiophenol (2.02 mL, 19.77 mmol), potassium carbonate (2.13 g, 15.40 mmol), and an additional 20 mL dry DMF were added, and the reaction was allowed to proceed overnight again. The following morning, salts were removed from the supernatant via multiple rounds of centrifugation and rinsing with DMF. The combined supernatants were concentrated in vacuo to an oil, which was taken up in 40 mL DCM, washed with water (2×10 mL) and brine (2×10 mL), dried over potassium carbonate, filtered, and concentrated to an oil. The oil was taken up again in DCM and purified via silica gel chromatography in DCM with a 0-50% (50:45:5 DCM/MeOH/concentrated aqueous ammonium hydroxide) gradient. Fractions containing product were combined and concentrated to give di-tert-butyl ((butane-1,4-diylbis(azanediyl))bis(3-ethylpentane-1,3-diyl))dicarbamate as a colorless oil (2.14 g, 4.17 mmol, 81.1%). UPLC/ELSD: RT=2.52 min. MS (ES): m/z (MH⁺) 515.6 for C₂₈H₅₈N₄O₄. ¹H NMR (300 MHz, CDCl₃) δ: ppm 5.20 (m, 2H), 2.45 (br. m, 8H), 1.48 (m, 13H), 1.35 (s, 4H), 1.23 (s, 19H), 0.62 (t, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate

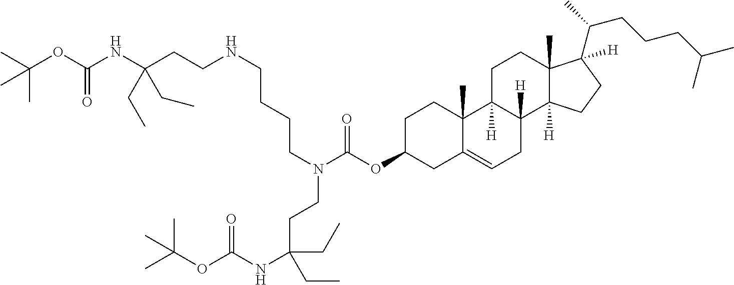

To a solution of di-tert-butyl ((butane-1,4-diylbis(azanediyl))bis(3-ethylpentane-1,3-diyl))dicarbamate (0.50 g, 0.97 mmol) in dry toluene (10 mL) set stirring under nitrogen was added triethylamine (0.41 mL, 2.91 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.54 g, 0.97 mmol) was added and the solution was heated to 90° C. and allowed to proceed overnight. The following morning, the reaction mixture was allowed to cool to room temperature, diluted with toluene, washed with water (3×15 mL), dried over sodium sulfate, filtered, and concentrated to an oil. The oil was taken up in DCM and purified via silica gel chromatography in DCM with a 0-30% (50:45:5 DCM/MeOH/concentrated aqueous ammonium hydroxide) gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate as a colorless oil (0.55 g, 0.59 mmol, 60.9%). UPLC/ELSD: RT=3.06 min. MS (ES): m/z (MH⁺) 928.3 for C₅₆H₁₀₂N₄O₆. ¹H NMR (300 MHz, CDCl₃) δ: ppm 5.34 (br. m, 1H), 5.02 (m, 1H), 4.46 (br. m, 3H), 3.18 (br. m, 4H), 2.56 (m, 4H), 2.28 (m, 2H), 1.83 (m, 6H), 1.58 (br. m, 16H), 1.39 (s, 18H), 1.10 (br. m, 11H), 0.97 (s, 5H), 0.88 (d, 3H, J=6 Hz), 0.79 (m, 18H), 0.64 (s, 4H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

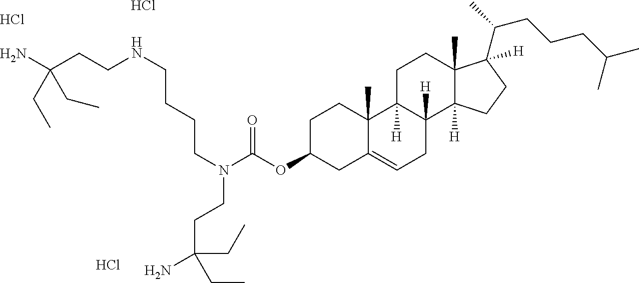

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-((tert-butoxycarbonyl)amino)-3-ethylpentyl)amino)butyl)carbamate (0.55 g, 0.59 mmol) in isopropanol (10 mL) set stirring under nitrogen was added hydrochloric acid (5 N in isopropanol, 1.18 mL, 5.90 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride as a white solid (0.34 g, 0.40 mmol, 68.1%). UPLC/ELSD: RT=2.08 min. MS (ES): m/z (MH⁺) 728.2 for C₄₆H₈₉Cl₃N₄O₂. ¹H NMR (300 MHz, MeOD) δ: ppm 5.45 (m, 1H), 4.48 (br. m, 1H), 3.94 (m, 1H), 3.37 (br. m, 3H), 3.14 (m, 4H), 2.40 (m, 2H), 2.11 (m, 3H), 1.93 (br. m, 6H), 1.74 (br. m, 13H), 1.55 (m, 12H), 1.18 (d, 14H, J=6 Hz), 1.04 (br. m, 17H), 0.98 (d, 4H, J=6 Hz), 0.91 (d, 6H, J=6 Hz), 0.74 (s, 3H).

### BI. Compound SA134: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

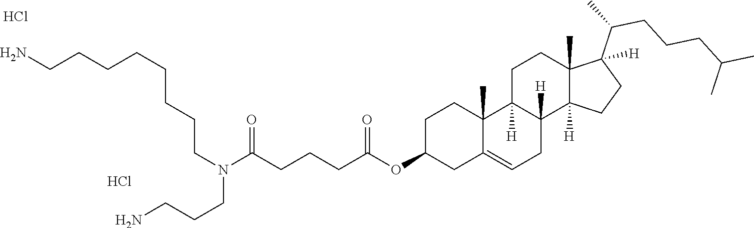

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate

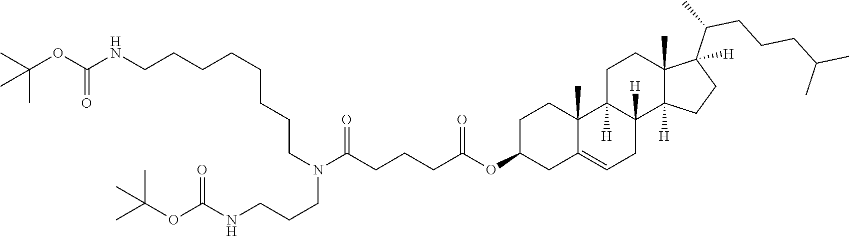

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.24 g, 0.48 mmol) in dry DCM (10 mL) stirring under nitrogen was added tert-butyl N-[3-({8-[(tert-butoxycarbonyl)amino]octyl}amino)propyl]carbamate (0.19 g, 0.48 mmol), dimethylaminopyridine (0.12 g, 0.95 mmol), and 1-ethyl-3-(3-dimethylaminopropyl)carbodiimide hydrochloride (0.18 g, 0.95 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate as a light yellow oil (0.32 g, 0.37 mmol, 77.0%). UPLC/ELSD: RT: 3.52 min. MS (ES): m/z (MH⁺) 885.4 for C₅₃H₉₃N₃O₇. ¹H NMR (300 MHz, CDCl₃) δ: ppm 5.17 (m, 1H), 5.02 (m, 1H), 4.43 (br. m, 1H), 4.29 (br. m, 1H), 3.06 (m, 2H), 2.95 (m, 1H), 2.86 (m, 2H) 2.73 (m, 4H), 2.04 (br. m, 6H), 1.62 (br. m, 4H), 1.51 (m, 4H), 1.20 (br. m, 12H), 1.10 (s, 19H), 0.97 (br. m, 13H), 0.81 (br. m, 7H), 0.68 (s, 6H), 0.60 (d, 4H, J=6 Hz), 0.54 (d, 6H, J=6 Hz), 0.35 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

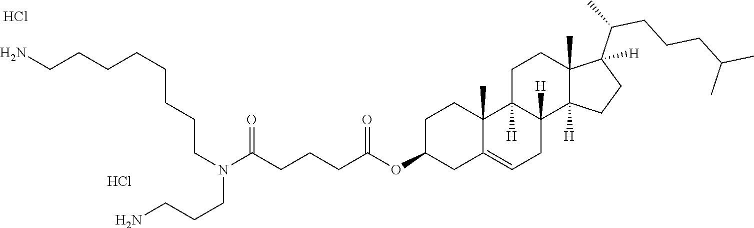

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate (0.32 g, 0.37 mmol) in isopropanol (5 mL) set stirring under nitrogen was added hydrochloric acid (5 N in isopropanol, 0.73 mL, 3.66 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride as a white solid (0.16 g, 0.19 mmol, 53.1%). UPLC/ELSD: RT=1.99 min. MS (ES): m/z (MH⁺) 684.9 for C₄₃H₇₉Cl₂N₃O₃. ¹H NMR (300 MHz, MeOD) δ: ppm 5.42 (m, 1H), 4.55 (br. m, 1H), 3.48 (t, 2H), 3.36 (m, 2H), 2.94 (m, 4H), 2.49 (t, 2H), 2.40 (m, 4H), 1.92 (br. m, 9H), 1.63 (br. m, 11H), 1.41 (br. m, 12H), 1.16 (m, 8H), 1.07 (s, 5H), 0.97 (d, 4H, J=6 Hz), 0.90 (d, 6H, J=6 Hz), 0.75 (s, 3H).

### BJ. Compound SA135: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

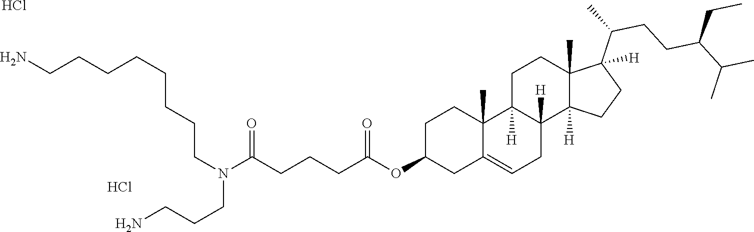

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate

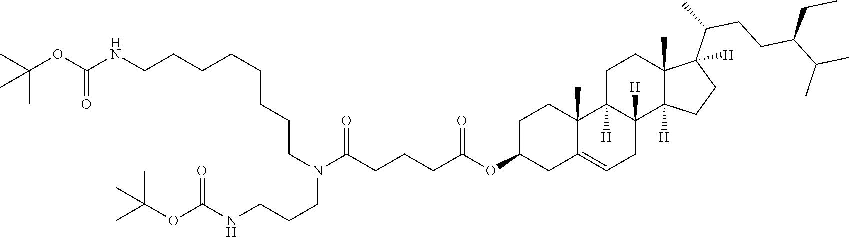

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl]oxy}-5-oxopentanoic acid (0.24 g, 0.45 mmol) in dry DCM (10 mL) stirring under nitrogen was added tert-butyl N-[3-({8-[(tert-butoxycarbonyl)amino]octyl}amino)propyl]carbamate (0.18 g, 0.45 mmol), dimethylaminopyridine (0.11 g, 0.90 mmol), and 1-ethyl-3-(3-dimethylaminopropyl)carbodiimide hydrochloride (0.17 g, 0.90 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate as a light yellow oil (0.27 g, 0.30 mmol, 67.0%). UPLC/ELSD: RT: 3.60 min. MS (ES): m/z (MH⁺) 913.4 for C₅₅H₉₇N₃O₇. ¹H NMR (300 MHz, CDCl₃) δ: ppm 5.03 (m, 1H), 4.44 (m, 1H), 4.25 (br. m, 1H), 3.06 (br. m, 2H), 2.95 (m, 1H), 2.86 (m, 2H) 2.74 (m, 4H), 2.04 (br. m, 6H), 1.62 (br. m, 4H), 1.50 (m, 4H), 1.32 (br. m, 11H), 1.10 (s, 19H), 0.97 (br. m, 12H), 0.79 (br. m, 7H), 0.68 (s, 5H), 0.60 (d, 5H, J=6 Hz), 0.51 (q, 9H), 0.35 (s, 4H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride

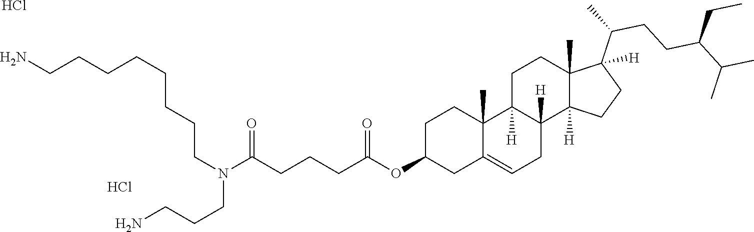

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-((tert-butoxycarbonyl)amino)octyl)(3-((tert-butoxycarbonyl)amino)propyl)amino)-5-oxopentanoate (0.27 g, 0.30 mmol) in isopropanol (5 mL) set stirring under nitrogen was added hydrochloric acid (5 N in isopropanol, 0.59 mL, 2.96 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. The white solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((8-aminooctyl)(3-aminopropyl)amino)-5-oxopentanoate dihydrochloride as a white solid (0.12 g, 0.12 mmol, 44.6%). UPLC/ELSD: RT=2.23 min. MS (ES): m/z (MH⁺) 712.8 for C₄₅H₈₃Cl₂N₃O₃. ¹H NMR (300 MHz, MeOD) δ: ppm 5.40 (m, 1H), 4.55 (br. m, 1H), 3.48 (t, 2H), 3.33 (m, 1H), 2.91 (m, 3H), 2.49 (t, 2H), 2.40 (m, 4H), 1.91 (br. m, 7H), 1.66 (br. m, 11H), 1.41 (br. m, 15H), 1.18 (d, 6H, J=6 Hz), 1.07 (s, 6H), 0.99 (d, 5H, J=6 Hz), 0.89 (q, 9H), 0.75 (s, 3H).

### BK. Compound SA136: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

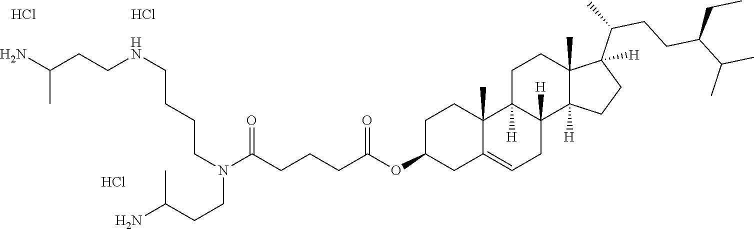

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate

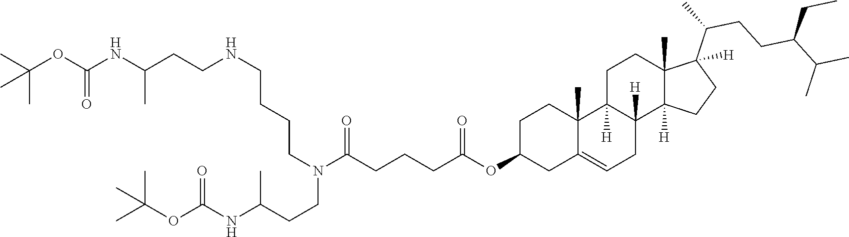

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl]oxy}-5-oxopentanoic acid (0.33 g, 0.61 mmol) in dry DCM (10 mL) stirring under nitrogen was added tert-butyl N-(4-{[4-({3-[(tert-butoxycarbonyl)amino]butyl}amino)butyl]amino}butan-2-yl)carbamate (0.79 g, 1.83 mmol), dimethylaminopyridine (0.15 g, 1.22 mmol), and 1-ethyl-3-(3-dimethylaminopropyl)carbodiimide hydrochloride (0.24 g, 1.22 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate as a light yellow oil (0.12 g, 0.12 mmol, 20.2%). UPLC/ELSD: RT: 2.81 min. MS (ES): m/z (MH⁺) 942.4 for C₅₆H₁₀₀N₄O₇. ¹H NMR (300 MHz, CDCl₃) δ: ppm 5.33 (m, 1H), 4.85 (m, 3H), 3.70 (br. m, 1H), 3.23 (br. m, 5H), 2.56 (br. m, 4H), 2.32 (m, 7H) 1.91 (m, 8H), 1.56 (br. m, 12H), 1.40 (s, 21H), 1.21 (m, 6H), 1.12 (m, 11H), 0.98 (s, 5H), 0.90 (d, 5H, J=6 Hz), 0.79 (q, 9H), 0.65 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride

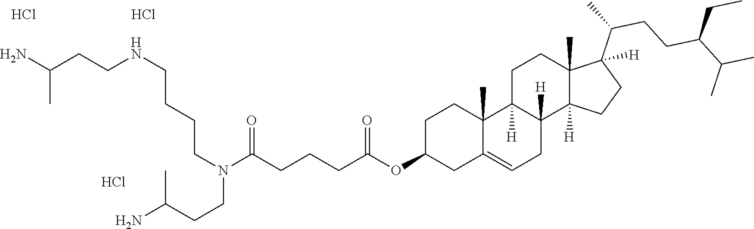

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazanonadecan-19-oate (0.12 g, 0.12 mmol) in isopropanol (5 mL) set stirring under nitrogen was added hydrochloric acid (5 N in isopropanol, 0.25 mL, 1.23 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(4-((3-aminobutyl)amino)butyl)amino)-5-oxopentanoate trihydrochloride as a white solid (0.05 g, 0.05 mmol, 42.5%). UPLC/ELSD: RT=1.90 min. MS (ES): m/z (MH⁺) 742.0 for C₄₆H₈₇Cl₃N₄O₃. ¹H NMR (300 MHz, MeOD) δ: ppm 5.42 (m, 1H), 4.57 (br. m, 1H), 3.67 (m, 1H), 3.48 (m, 5H), 3.18 (m, 5H), 2.42 (m, 6H), 1.92 (br. m, 22H), 1.39 (m, 10H), 1.20 (m, 8H), 1.07 (s, 5H), 0.98 (d, 5H, J=6 Hz), 0.87 (q, 9H), 0.75 (s, 3H).

### BL. Compound SA137: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

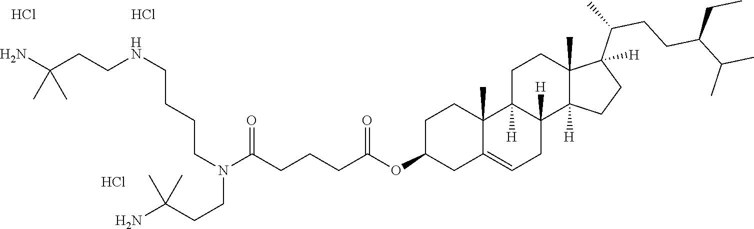

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate

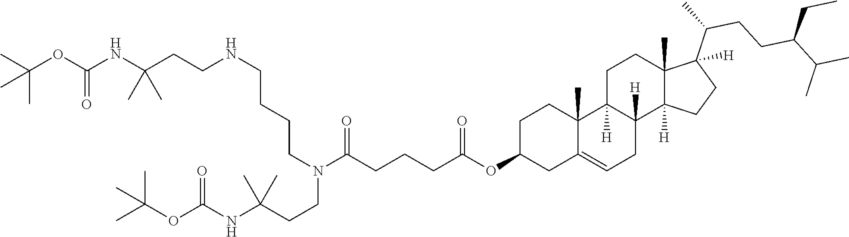

To a solution of 5-{[(1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl]oxy}-5-oxopentanoic acid (0.31 g, 0.58 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.80 g, 1.75 mmol), dimethylaminopyridine (0.14 g, 1.17 mmol), and 1-ethyl-3-(3-dimethylaminopropyl)carbodiimide hydrochloride (0.23 g, 1.17 mmol). The resulting solution was stirred at room temperature and proceeded overnight. Then the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate as a light yellow oil (0.20 g, 0.20 mmol, 35.0%). UPLC/ELSD: RT: 2.86 min. MS (ES): m/z (MH⁺) 970.4 for C₅₈H₁₀₄N₄O₇. ¹H NMR (300 MHz, CDCl₃) δ: ppm 5.54 (m, 1H), 5.04 (m, 1H), 4.23 (m, 2H), 2.91 (br. m, 4H), 2.35 (br. m, 4H), 2.03 (br. m, 6H), 1.61 (m, 8H) 1.35 (m, 10H), 1.10 (s, 19H), 0.94 (m, 15H), 0.83 (m, 6H), 0.68 (s, 6H), 0.60 (m, 5H), 0.49 (q, 9H), 0.35 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride

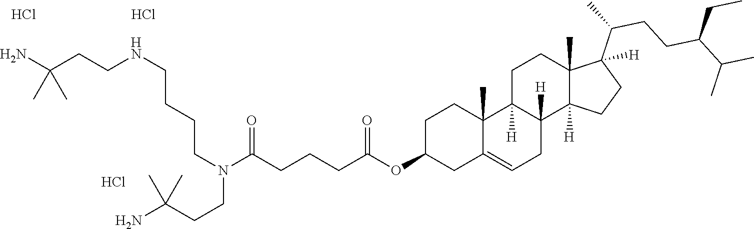

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazanonadecan-19-oate (0.20 g, 0.20 mmol) in isopropanol (5 mL) set stirring under nitrogen was added hydrochloric acid (5 N in isopropanol, 0.41 mL, 2.04 mmol) dropwise. The solution was heated to 40° C. and allowed to proceed overnight. The following morning, the mixture was cooled to room temperature and dry acetonitrile (20 mL) was added to the mixture, which was sonicated and allowed to stir for an additional hour. White solid was then filtered out of the solution, washed repeatedly with acetonitrile, and dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(4-((3-amino-3-methylbutyl)amino)butyl)amino)-5-oxopentanoate trihydrochloride as a white solid (0.11 g, 0.11 mmol, 54.0%). UPLC/ELSD: RT=1.94 min. MS (ES): m/z (MH⁺) 770.0 for C₄₈H₉₁Cl₃N₄O₃. ¹H NMR (300 MHz, MeOD) δ: ppm 5.42 (m, 1H), 4.55 (br. m, 1H), 3.45 (m, 4H), 3.16 (m, 4H), 2.41 (m, 6H), 1.89 (br. m, 22H), 1.43 (m, 14H), 1.27 (m, 7H), 1.18 (m, 4H), 1.07 (s, 6H), 0.98 (d, 5H, J=6 Hz), 0.89 (q, 9H), 0.75 (s, 3H).

### BM. Compound SA138: N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

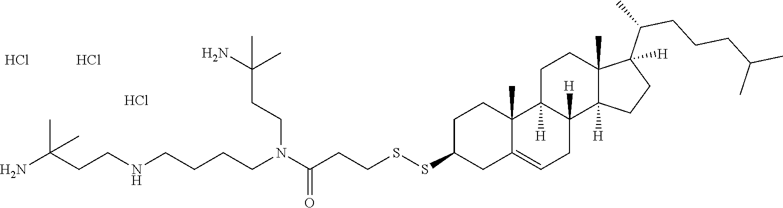

### Step 1: tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate

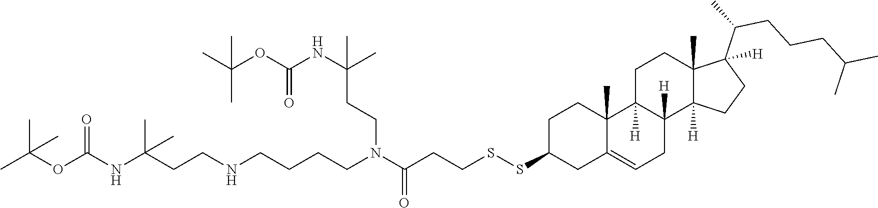

A solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.250 g, 0.493 mmol), tert-butyl N-(4-{[4-({3-[(tert-butoxycarbonyl)amino]-3-methylbutyl}amino)butyl]amino}-2-methylbutan-2-yl)carbamate (0.452 g, 0.986 mmol), and triethylamine (0.20 mL, 1.4 mmol) in DCM (2.5 mL) was cooled to 0° C. in an ice bath, and then propanephosphonic acid anhydride (50 wt % in DCM) (0.62 g, 0.97 mmol) was added dropwise. The reaction mixture stirred at rt and was monitored by LCMS. At 1.5 h, the reaction mixture was cooled to 0° C. in an ice bath, and 5% aq. NaHCO₃ solution (10 mL) was added. The reaction mixture then stirred at rt for 10 min. After this time, the mixture was extracted with DCM (3×15 mL). The combined organics were passed through a hydrophobic frit, dried over Na₂SO₄, and concentrated. The crude material was purified via silica gel chromatography (0-12% (5% conc. aq. NH₄OH in MeOH) in DCM) to afford tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate (0.269 g, 0.284 mmol, 57.6%) as a clear gel. UPLC/ELSD: RT=2.90 min. MS (ES): m/z=948.55 [M+H]⁺ for C₅₄H₉₈N₄O₅S₂. ¹H NMR (300 MHz, CDCl₃): δ 5.32-5.39 (m, 1H), 3.18-3.58 (m, 6H), 2.43-3.03 (m, 9H), 2.26-2.40 (m, 2H), 0.91-2.18 (br. m, 64H), 1.00 (s, 3H), 0.91 (d, 3H, J=6.5 Hz), 0.86 (d, 3H, J=6.6 Hz), 0.86 (d, 3H, J=6.6 Hz), 0.67 (s, 3H).

### Step 2: N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

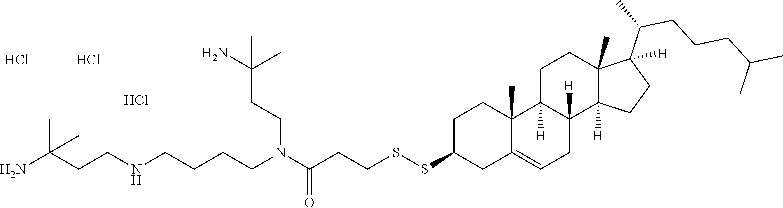

To a solution of tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6,6,17-pentamethyl-4-oxo-3-oxa-5,9,14-triazaoctadecan-17-yl)carbamate (0.266 g, 0.281 mmol) in DCM (2.6 mL) in a screwcap vial was added 4 N HCl in dioxane (0.49 mL). The reaction mixture stirred at rt and was monitored by LCMS. At 2 h, the reaction mixture was diluted with MTBE to 30 mL, and then centrifuged (10,000×g for 30 min). The supernatant was decanted. The solids were suspended in MTBE and then concentrated to afford N-(3-amino-3-methylbutyl)-N-(4-((3-amino-3-methylbutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride (0.203 g, 0.225 mmol, 80.3%) as a white solid. UPLC/ELSD: RT=1.96 min. MS (ES): m/z=264.75 [(M+3H)+CH₃CN]³⁺ for C₄₄H₈₂N₄OS₂. ¹H NMR (300 MHz, CD₃OD): δ 5.35-5.42 (m, 1H), 3.37-3.59 (m, 4H), 3.05-3.25 (m, 4H), 2.92-3.03 (m, 2H), 2.76-2.89 (m, 2H), 2.57-2.74 (m, 1H), 2.25-2.42 (m, 2H), 0.96-2.18 (br. m, 46H), 1.03 (s, 3H), 0.95 (d, 3H, J=6.5 Hz), 0.88 (d, 6H, J=6.6 Hz), 0.72 (s, 3H).

### BN. Compound SA139: N-(3-amino-3-methylbutyl)-N-(8-amino-8-methylnonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

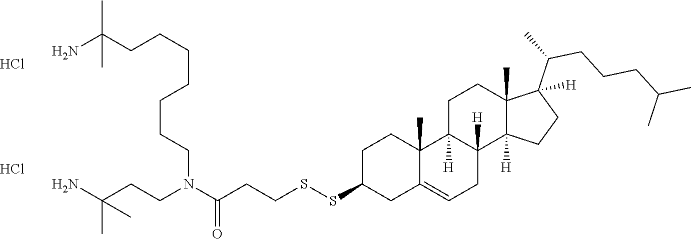

### Step 1: 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate

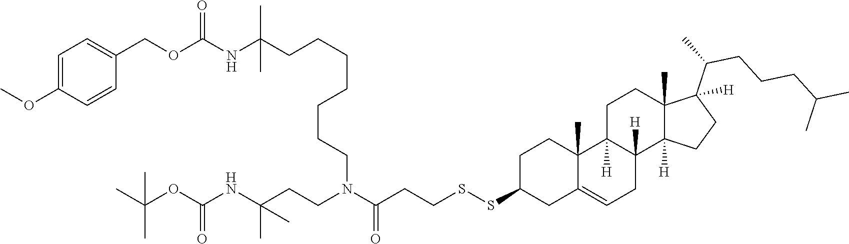

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.100 g, 0.197 mmol), tert-butyl N-(4-{[8-({[(4-methoxyphenyl)methoxy]carbonyl}amino)-8-methylnonyl]amino}-2-methylbutan-2-yl)carbamate (0.103 g, 0.197 mmol), and triethylamine (0.09 mL, 0.6 mmol) in DCM (1.0 mL) cooled to 0° C. was added 50 wt % propanephosphonic acid anhydride in DCM (0.20 mL, 0.39 mmol) dropwise. The reaction mixture was stirred at room temperature and was monitored by LCMS. At 16 hours, the reaction mixture was diluted with DCM (10 mL), and then washed with 5% aq. NaHCO₃ soln. The aqueous layer was extracted with DCM (10 mL). The combined organic layers were passed through a hydrophobic frit, dried over Na₂SO₄, and concentrated. The crude material was purified via silica gel chromatography (0-50% EtOAc in hexanes) to afford 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate (0.146 g, 0.144 mmol, 73.2%) as a clear oil. UPLC/ELSD: RT=3.80 min. MS (ES): m/z=1012.83 (M+H)⁺ for C₅₉H₉₉N₃O₆S₂. ¹H NMR (300 MHz, CDCl₃) δ 7.32-7.26 (m, 2H), 6.93-6.84 (m, 2H), 5.40-5.31 (m, 1H), 4.97 (s, 2H), 4.73-4.34 (m, 2H), 3.80 (s, 3H), 3.37-3.17 (m, 4H), 3.03-2.88 (m, 2H), 2.76-2.56 (m, 3H), 2.42-2.25 (m, 2H), 2.12-0.94 (m, 61H), 0.99 (s, 3H), 0.91 (d, J=6.4 Hz, 3H), 0.86 (d, J=6.6 Hz, 6H), 0.67 (s, 3H).

### Step 2: N-(3-amino-3-methylbutyl)-N-(8-amino-8-methylnonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

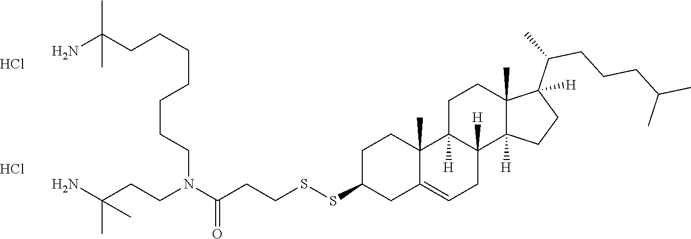

To a stirred solution of 4-methoxybenzyl (9-(N-(3-((tert-butoxycarbonyl)amino)-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylnonan-2-yl)carbamate (0.143 g, 0.142 mmol) in DCM (2.5 mL) cooled to 0° C. was added 4 N HCl in dioxane (0.25 mL). The reaction mixture was allowed to come to room temperature slowly while stirring and was monitored by LCMS. At 22 hours, 4 N HCl in dioxane (0.10 mL) was added. At 27 hours, MTBE (20 mL) added, and the reaction mixture was held at 4° C. overnight. The suspension was centrifuged (10,000×g for 30 min at 4° C.). The supernatant was decanted, the solids were suspended in MTBE, then concentrated to afford N-(3-amino-3-methylbutyl)-N-(8-amino-8-methylnonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.067 g, 0.078 mmol, 55.0%) as a white solid. UPLC/ELSD: RT=2.33 min. MS (ES): m/z=374.56 (M+2H)²⁺ for C₄₅H₈₃N₃OS₂. ¹H NMR (300 MHz, MeOD) δ 5.45-5.33 (m, 1H), 3.53-3.34 (m, 4H), 3.01-2.89 (m, 2H), 2.85-2.75 (m, 2H), 2.74-2.57 (m, 1H), 2.40-2.27 (m, 2H), 2.14-1.77 (m, 7H), 1.73-0.97 (m, 33H), 1.37 (s, 6H), 1.33 (s, 6H), 1.03 (s, 3H), 0.95 (d, J=6.5 Hz, 3H), 0.89 (d, J=6.6 Hz, 3H), 0.88 (d, J=6.7 Hz, 3H), 0.73 (s, 3H).

### BO. Compound SA141: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

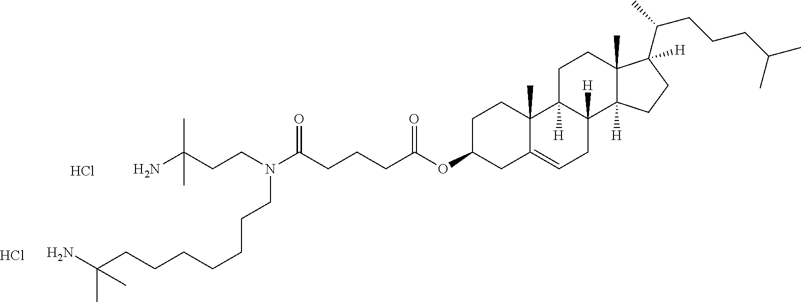

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate

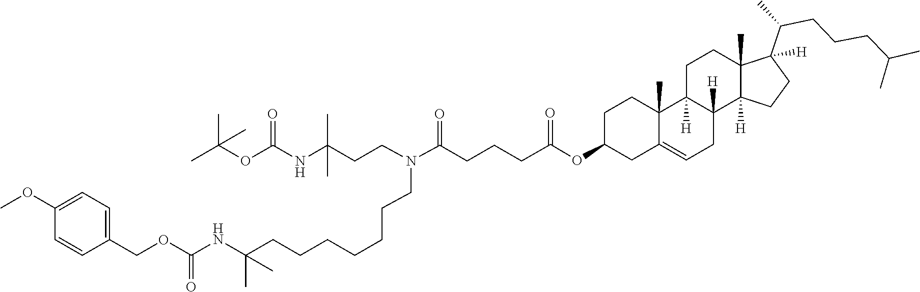

To a stirred solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.100 g, 0.200 mmol), tert-butyl N-(4-{[8-({[(4-methoxyphenyl)methoxy]carbonyl}amino)-8-methylnonyl]amino}-2-methylbutan-2-yl)carbamate (0.104 g, 0.200 mmol), and DMAP (0.049 g, 0.40 mmol) in DCM (2.0 mL) was added 1-ethyl-3-(3-dimethylaminopropyl)carbodiimide hydrochloride (0.077 g, 0.40 mmol). The reaction mixture was stirred at room temperature and was monitored by LCMS. At 15 hours, the reaction mixture was diluted with DCM (15 mL) and washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.158 g, 0.157 mmol, 78.8%) as a clear oil. UPLC/ELSD: RT=3.68 min. MS (ES): m/z=1005.92 (M+H)⁺ for C₆₁H₁₀₁N₃O₈. ¹H NMR (300 MHz, CDCl₃) δ 7.32-7.26 (m, 2H), 6.92-6.84 (m, 2H), 5.41-5.30 (m, 1H), 4.97 (s, 2H), 4.76-4.34 (m, 3H), 3.80 (s, 3H), 3.37-3.13 (m, 4H), 2.41-2.24 (m, 6H), 2.08-0.94 (m, 63H), 1.01 (s, 3H), 0.91 (d, J=6.4 Hz, 3H), 0.86 (d, J=6.6 Hz, 3H), 0.86 (d, J=6.6 Hz, 3H), 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

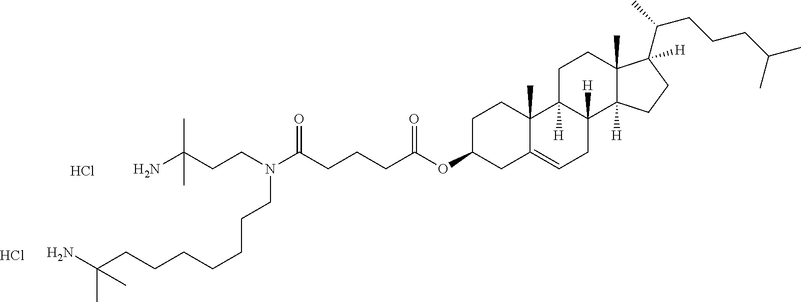

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.143 g, 0.143 mmol) in DCM (2.2 mL) was added 4 N HCl in dioxane (0.25 mL). The reaction mixture was stirred at room temperature and was monitored by LCMS. At 17 hours, 4 N HCl in dioxane (0.10 mL) was added. At 22 hours, MTBE (15 mL) added, and the reaction mixture was held at 4° C. overnight. The suspension was centrifuged (10,000×g for 30 min at 4° C.). The supernatant was decanted. The solids were suspended in MTBE and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride (0.082 g, 0.098 mmol, 68.7%) as a white solid. UPLC/ELSD: RT=2.24 min. MS (ES): m/z=371.23 (M+2H)²⁺ for C₄₇H₈₅N₃O₃. ¹H NMR (300 MHz, MeOD) δ 5.43-5.35 (m, 1H), 4.62-4.47 (m, 1H), 3.51-3.33 (m, 4H), 2.49-2.27 (m, 6H), 2.11-1.78 (m, 9H), 1.71-0.98 (m, 33H), 1.37 (s, 6H), 1.33 (s, 6H), 1.05 (s, 3H), 0.95 (d, J=6.4 Hz, 3H), 0.89 (d, J=6.6 Hz, 3H), 0.88 (d, J=6.6 Hz, 3H), 0.73 (s, 3H).

### BP. Compound SA142: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

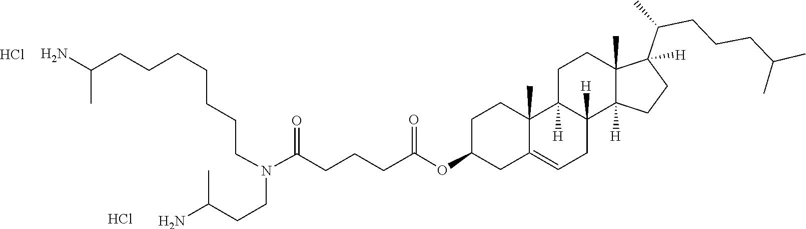

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate

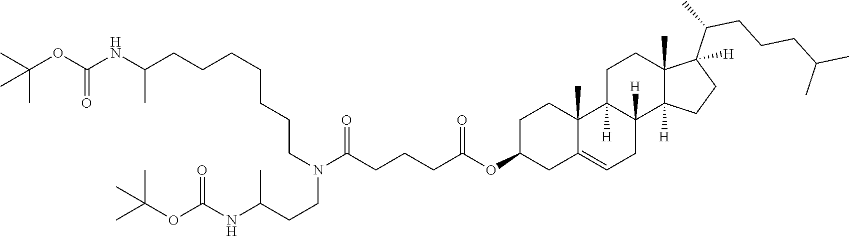

To a solution of 5-{[(1R,3aS,3bS,7S,9aR,9bS,11aR)-9a,11a-dimethyl-1-[(2R)-6-methylheptan-2-yl]-1H,2H,3H,3aH,3bH,4H,6H,7H,8H,9H,9bH,10H,11H-cyclopenta[a]phenanthren-7-yl]oxy}-5-oxopentanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate as a light yellow oil (0.13 g, 0.15 mmol, 82.9%). UPLC/ELSD: RT: 3.53 min. MS (ES): m/z (MH⁺) 913.4 for C₅₅H₉₇N₃O₇. ¹H NMR (300 MHz, CDCl₃) δ 5.35 (br. m, 1H), 4.59 (br. m, 2H), 4.38 (br. s, 1H), 3.59 (br. m, 2H), 3.22 (br. m, 4H), 2.32 (m, 6H), 1.93 (m, 4H), 1.81 (m, 3H), 1.50 (br. m, 10H), 1.42 (s, 18H), 1.27 (s, 14H), 1.09 (m, 12H), 0.99 (s, 6H), 0.90 (d, 4H, J=6 Hz), 0.83 (d, 6H, J=6 Hz), 0.66 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

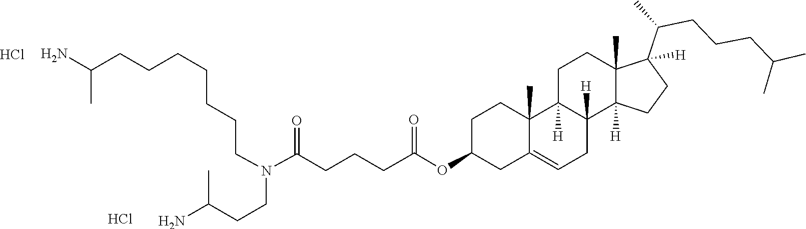

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate (0.13 g, 0.15 mmol) in DCM (3 mL) set stirring under nitrogen was added hydrochloric acid (4 N in dioxanes, 0.36 mL, 1.45 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride as a white solid (0.10 g, 0.12 mmol, 80.4%). UPLC/ELSD: RT=1.77 min. MS (ES): m/z (MH⁺) 713.3 for C₄₅H₈₃Cl₂N₃O₃. ¹H NMR (300 MHz, CDCl₃) δ 8.49 (br. m, 2H), 8.29 (br. m, 3H), 5.39 (s, 1H), 4.63 (br. m, 1H), 3.45 (br. m, 5H), 2.61 (br. m, 2H), 2.43 (br. m, 2H), 2.31 (d, 2H, J=9 Hz), 2.01 (br. m, 6H), 1.86 (br. m, 5H), 1.45 (br. m, 29H), 1.14 (br. m, 8H), 1.04 (s, 6H), 0.94 (d, 4H, J=6 Hz), 0.90 (d, 7H, J=6 Hz), 0.70 (s, 3H).

### BQ. Compound SA144: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

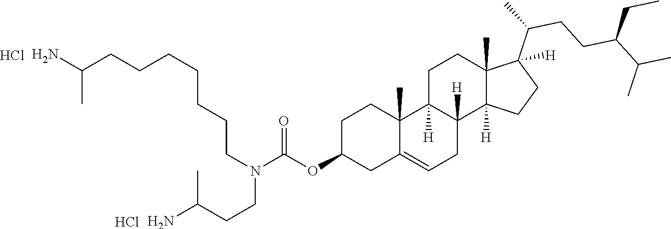

### Step 1: 8-bromo-N-methoxy-N-methyloctanamide

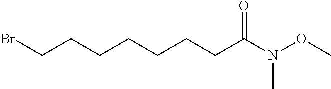

To a solution of 8-bromooctanoic acid (5.00 g, 22.41 mmol) in dry DCM (50 mL) stirring under nitrogen was added a solution of oxalyl chloride (16.81 mL, 33.62 mmol, 2M in DCM) dropwise. After the initial 3 mL of oxalyl chloride was added, catalytic dimethylformamide (0.17 mL, 2.24 mmol) was added, initiating gas formation seen by bubbling. Dropwise addition of the remainder of the oxalyl chloride solution followed. The reaction was allowed to proceed for 3 h at room temperature, and then the solution was concentrated in vacuo to a yellow oil. The residue was taken up in 30 mL DCM and added dropwise to a solution of N,O-dimethylhydroxylamine hydrochloride (2.69 g, 27.57 mmol) in 80 mL DCM, stirring under nitrogen. The solution was vented during addition as HCl gas was formed. The cloudy yellow reaction mixture was allowed to stir at room temperature overnight. Following, the mixture was diluted further with DCM, washed with water (1×30 mL), 1M HCl (1×30 mL), 1M NaOH (1×30 mL), and brine (1×30 mL), dried over sodium sulfate, filtered, and concentrated to give 8-bromo-N-methoxy-N-methyloctanamide as a clear yellow liquid used without further purification (5.87 g, 22.04 mmol, 98.4%). UPLC/ELSD: RT=0.67 min. MS (ES): m/z (MH⁺) 267.1 for C₁₀H₂₀BrNO₂. ¹H NMR (300 MHz, CDCl₃) δ: ppm 3.61 (s, 3H), 3.33 (t, 2H), 3.10 (s, 3H), 2.34 (t, 2H), 1.78 (qu, 2H), 1.56 (qu, 2H), 1.29 (br. m, 7H).

### Step 2: 9-bromononan-2-one

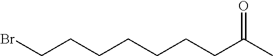

A solution of 8-bromo-N-methoxy-N-methyloctanamide (5.87 g, 22.04 mmol) in dry THE (100 mL) was set stirring under nitrogen and cooled to 0° C. Then, a solution of methylmagnesium bromide (11.02 ml, 33.06 mmol, 3M in diethyl ether) was added dropwise to the stirring mixture. The mixture was allowed to stir for 2.5 h at 0° C., and then allowed to gradually warm to room temperature for an additional 2 h. The mixture was then cooled again to 0° C., and the reaction was quenched with dropwise addition of hydrochloric acid (66.13 mL, 66.13 mmol, 1M). The mixture was allowed to continue stirring, gradually warming to room temperature over 30 minutes. The THF was removed under vacuum, and the mixture was extracted with EtOAc (3×50 mL). The combined organic phase was washed with brine (1×50 mL), dried over sodium sulfate, filtered, and concentrated in vacuo to an oil. The oil was taken up in DCM and purified on silica in hexanes with a 0-50% EtOAc gradient. Product-containing fractions were combined and concentrated to give 9-bromononan-2-one as a colorless oil (4.44 g, 20.08 mmol, 91.1%). UPLC/ELSD: RT=0.84 min. MS (ES): m/z (MH⁺) 222.1 for C₉H₁₇BrO. ¹H NMR (300 MHz, CDCl₃) δ: ppm 3.33 (t, 2H), 2.36 (t, 2H), 2.06 (s, 3H), 1.78 (qu, 2H), 1.50 (qu, 2H), 1.30 (br. m, 7H).

### Step 3: 9-bromononan-2-amine

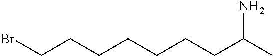

To a solution of 9-bromononan-2-one (3.44 g, 15.56 mmol) in dry MeOH (100 mL) was added ammonium acetate (10.79 g, 140.00 mmol) and sodium cyanoborohydride (1.27 g, 20.22 mmol). The solution was stirred vigorously for 36 h at room temperature. Following, the reaction was quenched with slow addition of HCl (100 mL, 2M). Then, 10 M NaOH was added dropwise until the pH of the solution reached 11-12, measured qualitatively with pH paper. Then, the mixture was extracted with DCM (3×150 mL), and the combined organic phase was washed with brine (1×100 mL), dried over sodium sulfate, filtered, and concentrated to a yellow oil. The oil was taken up in DCM and purified on silica in DCM with a 0-50% (1:1 DCM/MeOH) gradient. Product-containing fractions were pooled and concentrated in vacuo to give 9-bromononan-2-amine as a colorless oil (1.23 g, 5.53 mmol, 35.6%). UPLC/ELSD: RT=0.89 min. MS (ES): m/z (MH⁺) 223.1 for C₉H₂₀BrN. ¹H NMR (300 MHz, CDCl₃) δ: ppm 3.29 (t, 2H), 2.82 (br. m, 2H), 2.66 (s, 3H), 1.74 (qu, 2H), 1.21 (br. m, 11H), 0.99 (d, 3H).

### Step 4: tert-butyl (9-bromononan-2-yl)carbamate

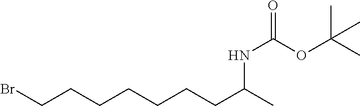 [SMILES: CC(CCCCCCCBr)NC(=O)OC(C)(C)C]

To a solution of 9-bromononan-2-amine (0.57 g, 2.54 mmol) in dry THE (10 mL) stirring under nitrogen at 0° C. was added di-tert-butyl dicarbonate (0.64 mL, 2.80 mmol). Then, triethylamine (0.39 mL, 2.80 mmol) was added dropwise, and the solution was allowed to gradually warm to room temperature and continue stirring overnight. Then solvent was removed under vacuum, and the resulting residue was taken up in DCM, washed with 5% aqueous HCl (1×15 mL) and water (1×15 mL), dried over sodium sulfate, filtered, and concentrated in vacuo to an oil. The oil was taken up in DCM and purified on silica in hexanes with a 0-20% EtOAc gradient. Product-containing fractions were pooled and concentrated in vacuo to give tert-butyl (9-bromononan-2-yl)carbamate as an oil (0.47 g, 1.47 mmol, 57.6%). UPLC/ELSD: RT=1.54 min. MS (ES): m/z (MH⁺) 323.3 for C₁₄H₂₈BrNO₂. ¹H NMR (300 MHz, CDCl₃) δ: ppm 4.35 (br. m, 1H), 3.60 (br. m, 1H), 3.37 (t, 2H), 1.84 (qu, 2H), 1.41 (br. s, 11H), 1.28 (br. m, 8H), 1.08 (d, 3H).

### Step 5: tert-butyl (9-((3-((tert-butoxycarbonyl)amino)butyl)amino)nonan-2-yl)carbamate

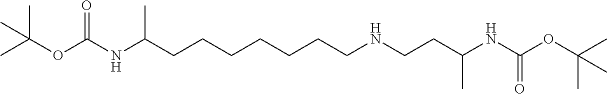 [SMILES: CC(CCCCCCCNCCC(C)NC(=O)OC(C)(C)C)NC(=O)OC(C)(C)C]

Both tert-butyl N-[4-(2-nitrobenzenesulfonamido)butan-2-yl]carbamate (0.88 g, 2.37 mmol) and tert-butyl (9-bromononan-2-yl)carbamate (0.76 g, 2.37 mmol) were dissolve in 20 mL dry DMF and set stirring under nitrogen. Then, potassium carbonate (1.96 g, 14.21 mmol) was added, and the solution was heated to 40° C. and allowed to stir overnight. The following morning, the mixture was cooled to room temperature, and benzyl bromide (0.17 mL, 1.42 mmol) was added. The solution stirred for 5 hours at room temperature. Then, thiophenol (0.727 mL, 7.10 mmol), potassium carbonate (0.98 g, 7.10 mmol), and an additional 10 mL dry DMF was added, and the reaction was allowed to stir for 2 days. Following, the salts were removed from the solution by centrifugation, and the supernatant was evaporated to a residue. The residue was taken up in 40 mL DCM and washed with water (2×10 mL) and brine (2×10 mL), dried over sodium sulfate, filtered, and concentrated to an oil. The oil was resuspended in DCM and purified on silica in DCM with a 0-50% (50:45:5 DCM/MeOH/aqueous NH₄OH) gradient. Product-containing fractions were pooled and concentrated to give tert-butyl (9-((3-((tert-butoxycarbonyl)amino)butyl)amino)nonan-2-yl)carbamate as a colorless oil (0.77 g, 1.80 mmol, 76.1%). UPLC/ELSD: RT=0.61 min. MS (ES): m/z (MH⁺) 430.6 for C₂₃H₄₇N₃O₄. ¹H NMR (300 MHz, CDCl₃) δ 4.94 (br. s, 1H), 4.33 (br. s, 1H), 3.62 (br. m, 2H), 2.61 (m, 4H), 1.66 (br. s, 1H), 1.45 (s, 21H), 1.29 (s, 9H), 1.14 (m, 6H).

### Step 6: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate

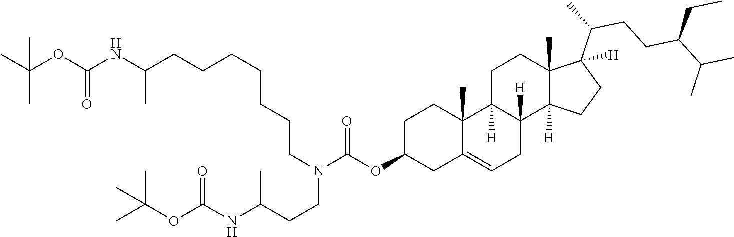

To a solution of tert-butyl (9-((3-((tert-butoxycarbonyl)amino)butyl)amino)nonan-2-yl)carbamate (0.11 g, 0.26 mmol) in dry toluene (5 mL) set stirring under nitrogen was added triethylamine (0.11 mL, 0.78 mmol). Then, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-nitrophenyl) carbonate (0.15 g, 0.26 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-30% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate as a light yellow oil (0.18 g, 0.21 mmol, 81.3%). UPLC/ELSD: RT=3.79 min. MS (ES): m/z (MH⁺) 871.4 for C₅₃H₉₅N₃O₆. ¹H NMR (300 MHz, CDCl₃) δ 5.07 (s, 1H), 4.14 (br. m, 3H), 3.33 (br. s, 2H), 2.91 (br. m, 4H), 2.07 (m, 2H), 1.66 (m, 6H), 1.26 (br. m, 10H), 1.14 (s, 21H), 0.98 (s, 15H), 0.83 (d, J=21.1 Hz, 12H), 0.73 (s, 6H), 0.65 (s, 5H), 0.56 (s, 9H), 0.39 (s, 3H).

### Step 7: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

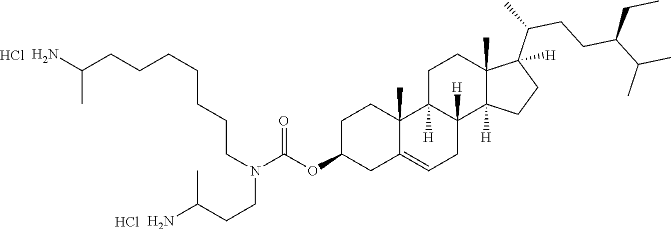

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate (0.18 g, 0.21 mmol) in DCM (3 mL) set stirring under nitrogen was added hydrochloric acid (4 N in dioxanes, 0.53 mL, 2.11 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-aminobutyl)(8-aminononyl)carbamate dihydrochloride as a white solid (0.14 g, 0.18 mmol, 83.4%). UPLC/ELSD: RT=1.89 min. MS (ES): m/z (MH⁺) 671.3 for C₄₃H₈₁Cl₂N₃O₂. ¹H NMR (300 MHz, CDCl₃) δ 8.52 (br. m, 3H), 8.33 (br. m, 3H), 5.40 (br. s, 1H), 4.52 (br. s, 1H), 3.33 (br. m, 6H), 2.37 (m, 2H), 2.01 (br. m, 7H), 1.34 (br. m, 31H), 1.18 (dd, J=13.1, 7.6 Hz, 4H), 1.11 (s, 2H), 1.04 (br. m, 4H), 0.93 (br. s, 4H), 0.85 (q, 8H), 0.68 (s, 3H).

### BR. Compound SA145: N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

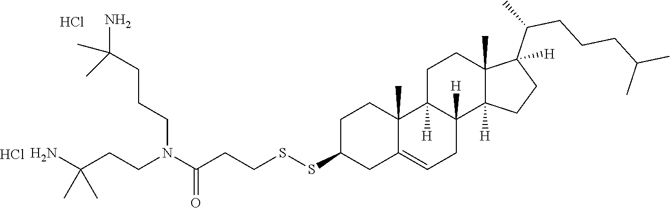

### Step 1: tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate

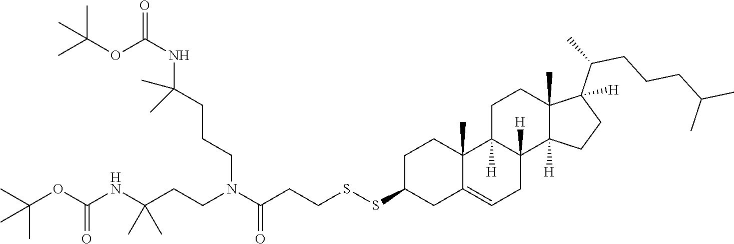

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.11 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate as a light yellow oil (0.17 g, 0.19 mmol, 86.7%). UPLC/ELSD: RT: 3.70 min. MS (ES): m/z (MH⁺) 891.4 for C₅₁H₉₁N₃O₅S₂. ¹H NMR (300 MHz, CDCl₃) δ 5.27 (br. s, 1H), 4.68 (s, 1H), 4.41 (br. s, 1H), 4.03 (q, 1H), 3.20 (br. m, 4H), 2.87 (br. m, 2H), 2.62 (br. m, 3H), 2.26 (br. m, 2H), 1.96 (br. m, 8H), 1.50 (br. m, 9H), 1.37 (s, 19H), 1.28 (br. m, 3H), 1.20 (m, 14H), 1.04 (br. m, 6H), 0.92 (s, 5H), 0.85 (d, 4H, J=6 Hz), 0.80 (d, 6H, J=6 Hz), 0.60 (s, 3H).

### Step 2: N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

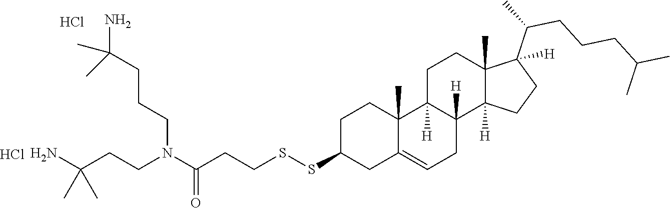

To a solution of tert-butyl (4-(N-(4-((tert-butoxycarbonyl)amino)-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)-2-methylbutan-2-yl)carbamate (0.17 g, 0.19 mmol) in DCM (5 mL) set stirring under nitrogen was added hydrochloric acid (4 M in dioxanes, 0.49 mL, 1.94 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give N-(3-amino-3-methylbutyl)-N-(4-amino-4-methylpentyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.13 g, 0.17 mmol, 85.9%). UPLC/ELSD: RT=2.12 min. MS (ES): m/z (MH⁺) 691.3 for C₄₁H₇₇Cl₂N₃OS₂. ¹H NMR (300 MHz, MeOD) δ 5.39 (br. s, 1H), 3.48 (br. m, 4H), 3.33 (br. s, 2H), 2.98 (br. m, 2H), 2.86 (br. m, 2H), 2.67 (br. m, 1H), 2.37 (d, 2H, J=6 Hz), 1.97 (br. m, 7H), 1.66 (br. m, 12H), 1.46 (s, 4H), 1.41 (s, 13H), 1.31 (s, 3H), 1.17 (br. m, 8H), 1.05 (s, 5H), 0.96 (d, 4H, J=6 Hz), 0.90 (d, 8H, J=6 Hz), 0.75 (s, 3H).

### BS. Compound SA149: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

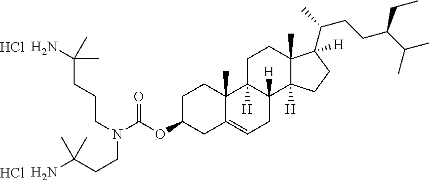

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate

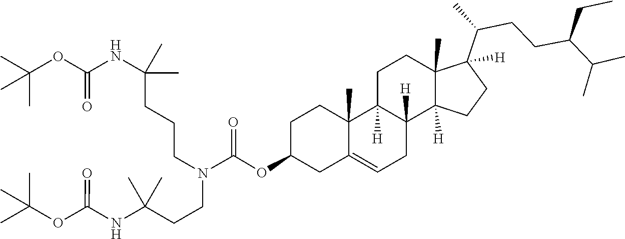

To a solution of tert-butyl (5-((3-((tert-butoxycarbonyl)amino)-3-methylbutyl)amino)-2-methylpentan-2-yl)carbamate (0.09 g, 0.22 mmol) in dry toluene (5 mL) set stirring under nitrogen was added triethylamine (0.07 mL, 0.09 mmol). Then, (1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,10H,11H-cyclopenta[a]phenanthren-7-yl 4-nitrophenyl carbonate (0.13 g, 0.22 mmol) was added, and the solution was heated to 90° C. and allowed to proceed for 2 days. Then, the reaction mixture was allowed to cool to room temperature, diluted with toluene, and washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate as a light yellow oil (0.14 g, 0.17 mmol, 74.7%). UPLC/ELSD: RT=3.78 min. MS (ES): m/z (MH⁺) 843.4 for C₅₁H₉₁N₃O₆. ¹H NMR (300 MHz, CDCl₃) δ 5.30 (br. s, 1H), 4.42 (br. m, 3H), 3.12 (br. s, 4H), 2.28 (br. m, 2H), 1.80 (br. m, 7H), 1.52 (br. m, 11H), 1.35 (s, 18H), 1.20 (s, 18H), 1.08 (br. m, 5H), 0.94 (s, 6H), 0.86 (d, 5H, J=6 Hz), 0.75 (q, 9H), 0.61 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

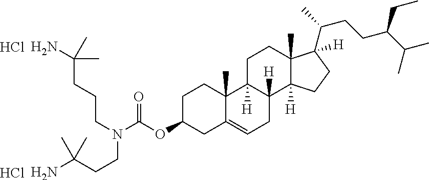

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate (0.14 g, 0.17 mmol) in DCM (5 mL) set stirring under nitrogen was added hydrochloric acid (4 M in dioxanes, 0.42 mL, 1.67 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride as a white solid (0.08 g, 0.10 mmol, 61.2%). UPLC/ELSD: RT=2.07 min. MS (ES): m/z (MH⁺) 643.3 for C₄₁H₇₇Cl₂N₃O₂. ¹H NMR (300 MHz, MeOD) δ 5.44 (br. s, 1H), 4.47 (br. m, 1H), 3.34 (br. m, 7H), 2.40 (br. m, 2H), 1.97 (br. m, 7H), 1.66 (br. m, 11H), 1.37 (d, 14H, J=6 Hz), 1.20 (br. m, 8H), 1.08 (s, 5H), 0.99 (d, 5H, J=6 Hz), 0.87 (q, 8H), 0.75 (s, 3H).

### BT. Compound SA151: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

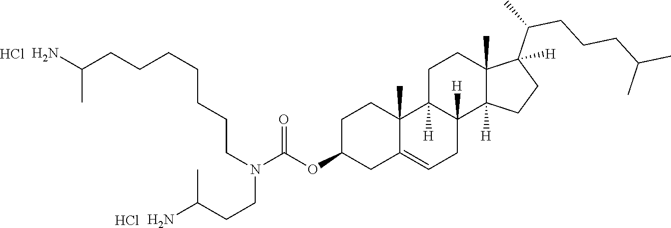

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate

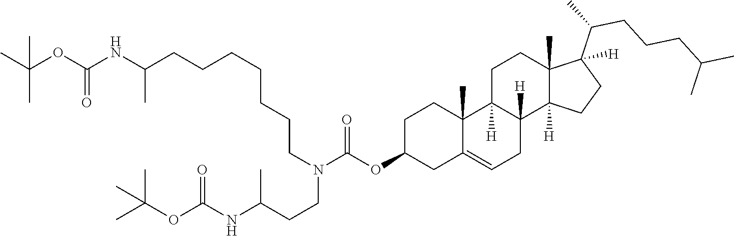

To a solution of tert-butyl (9-((3-((tert-butoxycarbonyl)amino)butyl)amino)nonan-2-yl)carbamate (0.12 g, 0.27 mmol) in dry toluene (5 mL) set stirring under nitrogen was added triethylamine (0.12 mL, 0.82 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.15 g, 0.27 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate as a light yellow oil (0.17 g, 0.21 mmol, 75.6%). UPLC/ELSD: RT=3.70 min. MS (ES): m/z (MH⁺) 843.4 for C₅₁H₉₁N₃O₆. ¹H NMR (300 MHz, CDCl₃) δ 5.35 (br. s, 1H), 4.46 (br. m, 3H), 3.59 (br. m, 2H), 3.19 (br. m, 4H), 2.29 (m, 2H), 2.01 (m, 6H), 1.59 (br. m, 10H), 1.40 (s, 20H), 1.25 (br. m, 15H), 1.10 (q, 12H), 0.99 (s, 6H), 0.90 (d, 4H, J=6 Hz), 0.83 (d, 6H, J=6 Hz), 0.65 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride

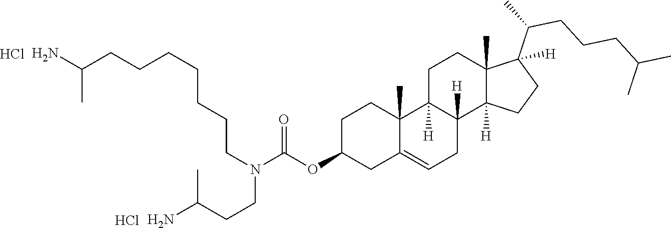

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)butyl)(8-((tert-butoxycarbonyl)amino)nonyl)carbamate (0.17 g, 0.21 mmol) in DCM (3 mL) set stirring under nitrogen was added hydrochloric acid (4 N in dioxanes, 0.52 mL, 2.05 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-aminobutyl)(8-aminononyl)carbamate dihydrochloride as a white solid (0.15 g, 0.20 mmol, 95.7%). UPLC/ELSD: RT=1.83 min. MS (ES): m/z (MH⁺) 643.3 for C₄₁H₇₇Cl₂N₃O₂. ¹H NMR (301 MHz, CDCl₃) δ 8.51 (br. s, 3H), 8.32 (br. s, 3H), 5.39 (br. m, 1H), 4.50 (br. m, 1H), 3.34 (br. m, 6H), 2.37 (m, 2H), 2.01 (br. m, 7H), 1.45 (br. m, 29H), 1.11 (br. m, 8H), 1.04 (s, 4H), 0.93 (d, 3H, J=6 Hz, 3H), 0.90 (d, 3H, J=6 Hz, 6H), 0.70 (s, 3H).

### BU. Compound SA152: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

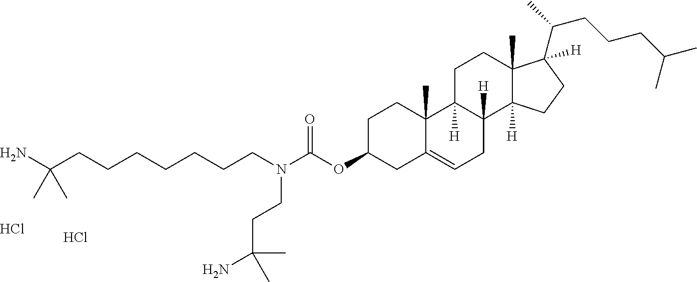

### Step 1: methyl 9-chloro-2,2-dimethylnonanoate

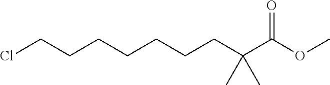

To a solution of THF (30 mL) and lithium diisopropylamide (19 mL, 2.0 M in THF) cooled to −78° C. was added methyl isobutyrate (3.0 mL, 26 mmol). The reaction mixture stirred at 0° C. for 50 min then was cooled to −78° C. 1-Bromo-7-chloroheptane (4.2 mL, 27 mmol) was added dropwise. The reaction mixture was stirred while slowly coming to room temperature and was monitored by TLC. At 20 hours, the reaction mixture was cooled to 0° C., and then aq. 1 N HCl (30 mL) was added dropwise. The biphasic mixture was separated, and the aqueous layer was extracted with EtOAc (2×30 mL). The combined organics were washed with brine, dried over Na₂SO₄, and concentrated to afford methyl 9-chloro-2,2-dimethylnonanoate (6.205 g, quant.) as an amber oil. Material was carried forward as is. ¹H NMR (300 MHz, CDCl₃): δ 3.65 (s, 3H), 3.52 (t, J=6.7 Hz, 2H), 1.83-1.63 (m, 2H), 1.63-1.17 (m, 10H), 1.15 (s, 6H).

### Step 2: 9-chloro-2,2-dimethylnonanoic acid

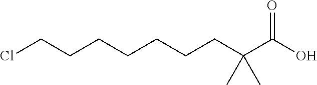

A mixture of methyl 9-chloro-2,2-dimethylnonanoate (6.2 g, 26 mmol), THE (60 mL), MeOH (45 mL), and aq. 10% NaOH (31 mL, 78 mmol) was stirred at 50° C. The reaction was monitored by TLC. At 23 hours, the reaction mixture was concentrated to remove volatile organics. The residue was taken up in water (70 mL), washed with MTBE (2×50 mL), and then acidified to pH˜1 with aq. 2 N HCl. The aqueous was extracted with EtOAc (3×50 mL), dried over Na₂SO₄, and then concentrated to afford 9-chloro-2,2-dimethylnonanoic acid (4.997 g, 22.64 mmol, 85.7%) as an amber oil. UPLC/ELSD: RT=1.00 min. MS (ES): m/z=174.98 (M-CO₂H)+ for C₁₁H₂₁ClO₂. ¹H NMR (300 MHz, CDCl₃): δ 9.71 (br. s, 1H), 3.53 (t, J=6.7 Hz, 2H), 1.88-1.66 (m, 2H), 1.62-1.22 (m, 10H), 1.19 (s, 6H).

### Step 3: (4-methoxyphenyl)methyl N-(9-chloro-2-methylnonan-2-yl)carbamate

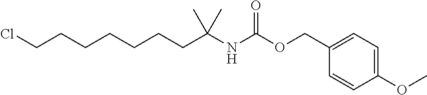 [SMILES: COc1ccc(COC(=O)NC(C)(C)CCCCCCCCl)cc1]

To a stirred solution of 9-chloro-2,2-dimethylnonanoic acid (2.00 g, 9.06 mmol) and triethylamine (1.8 mL, 13 mmol) in PhMe (30 mL) was added diphenylphosphoryl azide (2.4 mL, 11 mmol). The reaction mixture stirred at room temperature for 1.25 hours, then was stirred at 80° C. Gas evolution occurred. At 2 hours, the reaction mixture was cooled to room temperature, then washed with 5% aq. NaHCO₃ soln. (2×), water, and brine. The organics were dried over Na₂SO₄, and then 4-methoxybenzyl alcohol (2.2 mL, 18 mmol) and 1,8-diazabicyclo[5.4.0]undec-7-ene (2.8 mL, 19 mmol) were added sequentially. The reaction mixture was stirred at 80° C. and was monitored by LCMS. At 18 hours, the reaction mixture was cooled to room temperature, diluted with EtOAc (150 mL), washed with 5% aq. citric acid (2×), water, and brine, dried over Na₂SO₄, and concentrated. The crude material was purified via silica gel chromatography (0-30% EtOAc in hexanes) to afford (4-methoxyphenyl)methyl N-(9-chloro-2-methylnonan-2-yl)carbamate (1.613 g, 4.532 mmol, 50.0%) as a clear oil. UPLC/ELSD: RT=1.70 min. MS (ES): m/z=378.33 (M+Na)⁺ for C₁₉H₃₀ClNO₃. ¹H NMR (300 MHz, CDCl₃): δ 7.29 (d, J=8.3 Hz, 2H), 6.88 (d, J=8.1 Hz, 2H), 4.98 (s, 2H), 4.58 (s, 1H), 3.81 (s, 3H), 3.53 (t, J=6.7 Hz, 2H), 1.84-1.69 (m, 2H), 1.68-1.16 (m, 16H).

### Step 4: tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate

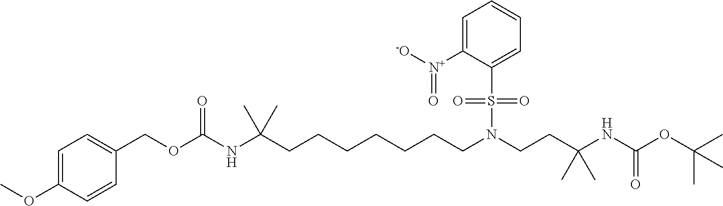

Tert-butyl N-[2-methyl-4-(2-nitrobenzenesulfonamido)butan-2-yl]carbamate (0.907 g, 2.34 mmol), (4-methoxyphenyl)methyl N-(9-chloro-2-methylnonan-2-yl)carbamate (0.700 g, 1.97 mmol), potassium carbonate (0.544 g, 3.93 mmol), potassium iodide (0.164 g, 0.983 mmol) and propionitrile (10.5 mL) were combined in a sealed tube. The reaction mixture was heated at 150° C. via microwave irradiation while stirring and was monitored by LCMS. At 12 hours, the reaction mixture was cooled to room temperature and filtered rinsing with ACN, and the filtrate was concentrated. The residue was taken up in EtOAc (100 mL), then washed with 5% aq. NaHCO₃ soln. and brine, dried over Na₂SO₄, and concentrated. The crude material was purified via silica gel chromatography (20-50% EtOAc in hexanes) to afford tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate (1.267 g, 1.792 mmol, 91.1%) as a yellow oil. UPLC/ELSD: RT=2.00 min. MS (ES): m/z=607.64 [(M+H)—(CH₃)₂C═CH₂—CO₂]⁺ for C₃₅H₅₄N₄O₉S. ¹H NMR (300 MHz, CDCl₃) δ 8.06-7.93 (m, 1H), 7.77-7.52 (m, 3H), 7.37-7.20 (m, 2H), 6.97-6.78 (m, 2H), 4.97 (s, 2H), 4.59 (s, 1H), 4.39 (s, 1H), 3.80 (s, 3H), 3.41-3.20 (m, 4H), 2.00-1.84 (m, 2H), 1.66-1.10 (m, 33H).

### Step 5: tert-butyl N-(4-{[8-({[(4-methoxyphenyl)methoxy]carbonyl}amino)-8-methylnonyl]amino}-2-methylbutan-2-yl)carbamate

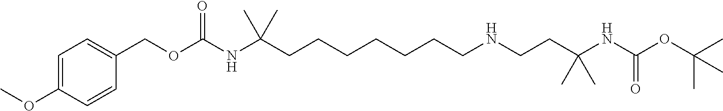 [SMILES: COc1ccc(COC(=O)NC(C)(C)CCCCCCCNCCC(C)(C)NC(=O)OC(C)(C)C)cc1]

To a mixture of tert-butyl N-(4-{N-[8-({[(4-methoxyphenyl)methoxy]carbonyl}amino)-8-methylnonyl]-2-nitrobenzenesulfonamido}-2-methylbutan-2-yl)carbamate (1.258 g, 1.780 mmol) and potassium carbonate (0.738 g, 5.34 mmol) in DMF (19 mL) was added thiophenol (0.33 mL, 3.2 mmol). The reaction mixture was stirred at room temperature and was monitored by LCMS. At 2 hours, the reaction mixture was filtered rinsing with EtOAc. The filtrate was diluted to 125 mL with EtOAc, washed with 5% aq. NaHCO₃ soln., water (3×), and brine, dried over Na₂SO₄, and concentrated. The crude material was purified via silica gel chromatography (0-16% (5% conc. aq. NH₄OH in MeOH) in DCM) to afford tert-butyl N-(4-{[8-({[(4-methoxyphenyl)methoxy]carbonyl}amino)-8-methylnonyl]amino}-2-methylbutan-2-yl)carbamate (0.699 g, 1.34 mmol, 75.3%) as a yellow oil. UPLC/ELSD: RT=0.89 min. MS (ES): m/z=522.74 (M+H)⁺ for C₂₉H₅₁N₃O₅. ¹H NMR (300 MHz, CDCl₃) δ 7.33-7.27 (m, 2H), 6.91-6.84 (m, 2H), 5.61 (s, 1H), 4.97 (s, 2H), 4.59 (s, 1H), 3.80 (s, 3H), 2.75 (t, J=7.2 Hz, 2H), 2.64 (t, J=7.3 Hz, 2H), 1.81 (t, J=7.2 Hz, 2H), 1.70-1.13 (m, 33H).

### Step 6: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate

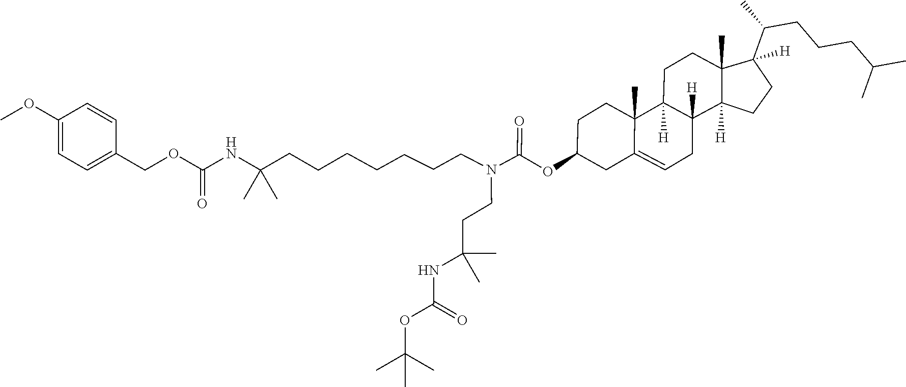

Cholesterol 4-nitrophenyl carbonate (0.150 g, 0.272 mmol), tert-butyl N-(4-{[8-({[(4-methoxyphenyl)methoxy]carbonyl}amino)-8-methylnonyl]amino}-2-methylbutan-2-yl)carbamate (0.184 g, 0.353 mmol), and triethylamine (0.12 mL, 0.86 mmol) were combined in PhMe (2.05 mL). The reaction mixture was stirred at 90° C. and was monitored by LCMS. At 20 hours, the reaction mixture was cooled to room temperature, diluted with DCM (30 mL), and then washed with 5% aq. NaHCO₃ soln. (3×). The organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.227 g, 0.243 mmol, 89.4%) as a clear oil. UPLC/ELSD: RT=3.79 min. MS (ES): m/z=935.72 (M+H)⁺ for C₅₇H₉₅N₃O₇. ¹H NMR (300 MHz, CDCl₃) δ 7.34-7.24 (m, 2H), 6.95-6.83 (m, 2H), 5.42-5.32 (m, 1H), 4.97 (s, 2H), 4.64-4.32 (m, 3H), 3.80 (s, 3H), 3.33-3.07 (m, 4H), 2.48-2.21 (m, 2H), 2.12-0.94 (m, 61H), 1.02 (s, 3H), 0.91 (d, J=6.4 Hz, 3H), 0.87 (d, J=6.6 Hz, 6H), 0.68 (s, 3H).

### Step 7: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

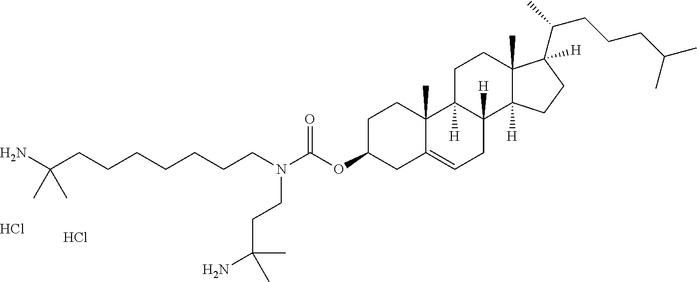

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.222 g, 0.238 mmol) in DCM (2.6 mL) was added 4 N HCl in dioxane (0.43 mL). The reaction mixture was stirred at room temperature and was monitored by LCMS. At 18 hours, hexanes (30 mL) was added, and the mixture was centrifuged (10,000×g for 30 min). The supernatant was decanted, the solids were suspended in hexanes (30 mL), and the suspension was centrifuged (10,000×g for 30 min). The supernatant was decanted, and the solids were dried under reduced pressure to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride (0.125 g, 0.163 mmol, 68.8%) as a white solid. UPLC/ELSD: RT=1.87 min. MS (ES): m/z=335.74 (M+2H)²⁺ for C₄₃H₇₉N₃O₂. ¹H NMR (300 MHz, DMSO) δ 8.29-7.87 (m, 6H), 5.41-5.26 (m, 1H), 4.39-4.22 (m, 1H), 3.29-3.06 (m, 4H), 2.36-2.11 (m, 2H), 2.09-0.90 (m, 52H), 0.98 (s, 3H), 0.89 (d, J=6.2 Hz, 3H), 0.84 (d, J=6.6 Hz, 6H), 0.65 (s, 3H).

### BV. Compound SA153: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

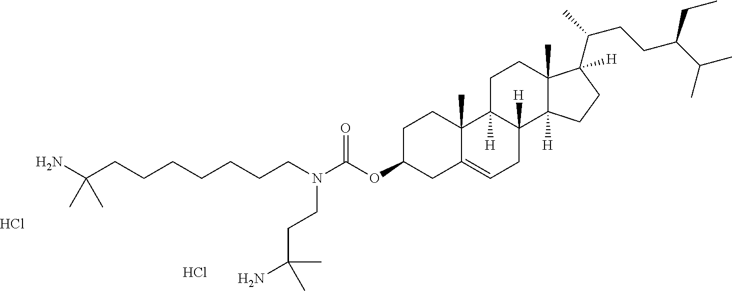

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate

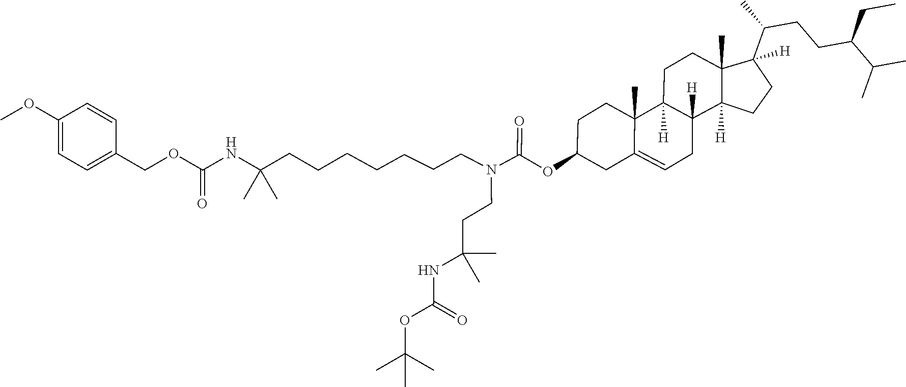

Sitosterol 4-nitrophenyl carbonate (0.175 g, 0.302 mmol), tert-butyl N-(4-{[8-({[(4-methoxyphenyl)methoxy]carbonyl}amino)-8-methylnonyl]amino}-2-methylbutan-2-yl)carbamate (0.205 g, 0.392 mmol), and triethylamine (0.13 mL, 0.93 mmol) were combined in PhMe (2.3 mL). The reaction mixture was stirred at 90° C. and was monitored by LCMS. At 20 hours, the reaction mixture was cooled to room temperature, diluted with DCM (30 mL), and then washed with 5% aq. NaHCO₃ soln. (3×). The organics were passed through a hydrophobic frit, dried over Na₂SO₄, and concentrated. The crude material was purified via silica gel chromatography (0-30% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.252 g, 0.262 mmol, 86.8%) as a clear oil. UPLC/ELSD: RT=3.89 min. MS (ES): m/z=963.23 (M+H)⁺ for C₅₉H₉₉N₃O₇. ¹H NMR (300 MHz, CDCl₃) δ 7.32-7.24 (m, 2H), 6.94-6.85 (m, 2H), 5.42-5.32 (m, 1H), 4.97 (s, 2H), 4.66-4.40 (m, 3H), 3.81 (s, 3H), 3.33-3.08 (m, 4H), 2.47-2.20 (m, 2H), 2.13-0.77 (m, 71H), 1.02 (s, 3H), 0.92 (d, J=6.3 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride

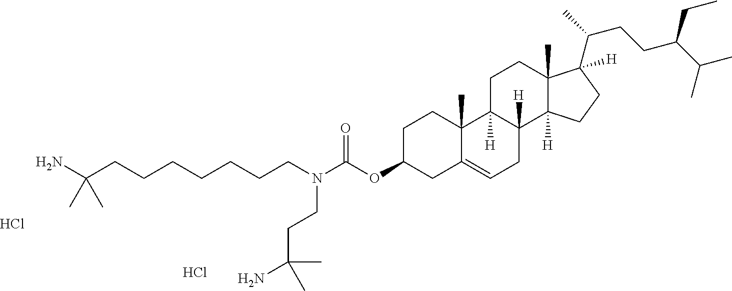

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(8-((((4-methoxybenzyl)oxy)carbonyl)amino)-8-methylnonyl)carbamate (0.248 g, 0.258 mmol) in DCM (2.6 mL) was added 4 N HCl in dioxane (0.46 mL). The reaction mixture was stirred at room temperature and was monitored by LCMS. At 18 hours, hexanes (30 mL) was added, and the mixture was centrifuged (10,000×g for 30 min). The supernatant was decanted, the solids suspended in hexanes (30 mL), and the suspension was centrifuged (10,000×g for 30 min). The supernatant was decanted, and the solids were dried under reduced pressure to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-methylbutyl)(8-amino-8-methylnonyl)carbamate dihydrochloride (0.107 g, 0.130 mmol, 50.6%) as a white solid. UPLC/ELSD: RT=3.89 min. MS (ES): m/z=370.68 [(M+2H)+CH₃CN]²⁺ for C₄₅H₈₃N₃O₂. ¹H NMR (300 MHz, DMSO) δ 8.34-7.91 (m, 6H), 5.39-5.29 (m, 1H), 4.41-4.21 (m, 1H), 3.30-3.07 (m, 4H), 2.37-2.16 (m, 2H), 2.05-0.74 (m, 62H), 0.98 (s, 3H), 0.90 (d, J=6.3 Hz, 3H), 0.65 (s, 3H).

### BW. Compound SA154: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

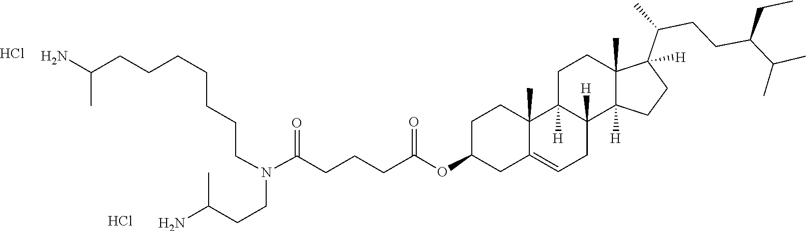

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate

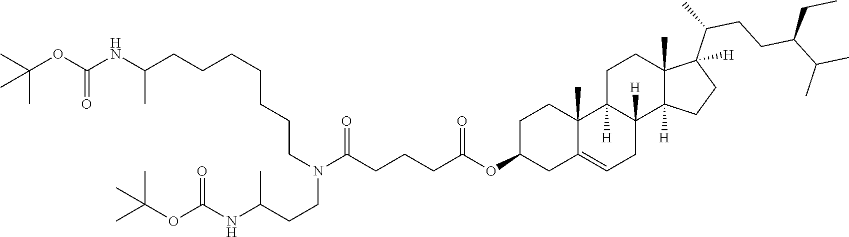

To a solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate as a light yellow oil (0.09 g, 0.10 mmol, 57.3%). UPLC/ELSD: RT: 3.63 min. MS (ES): m/z (MH⁺) 941.4 for C₅₇H₁₀₁N₃O₇. ¹H NMR (300 MHz, CDCl₃) δ 5.35 (br. s, 1H), 4.60 (br. m, 2H), 4.34 (br. m, 1H), 3.63 (br. m, 3H), 3.20 (br. m, 3H), 2.35 (m, 6H), 1.94 (m, 4H), 1.83 (br. m, 3H), 1.55 (br. m, 10H), 1.43 (s, 18H), 1.28 (br. m, 16H), 1.11 (m, 12H), 1.01 (s, 5H), 0.92 (d, 5H, J=6 Hz), 0.82 (q, 10H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride

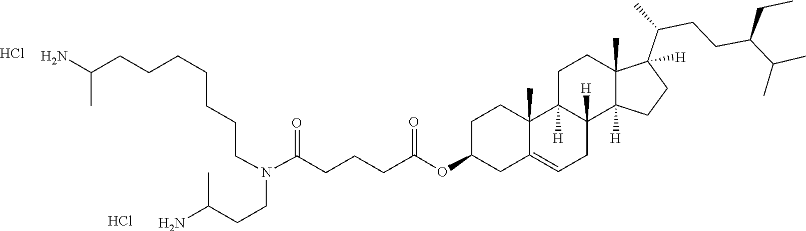

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)butyl)(8-((tert-butoxycarbonyl)amino)nonyl)amino)-5-oxopentanoate (0.09 g, 0.10 mmol) in DCM (3 mL) set stirring under nitrogen was added hydrochloric acid (4 N in dioxanes, 0.25 mL, 1.00 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-aminobutyl)(8-aminononyl)amino)-5-oxopentanoate dihydrochloride as a white solid (0.06 g, 0.06 mmol, 63.4%). UPLC/ELSD: RT=1.96 min. MS (ES): m/z (MH⁺) 741.3 for C₄₇H₈₇Cl₂N₃O₃. ¹H NMR (300 MHz, CDCl₃) δ 8.41 (m, 6H), 5.38 (br. s, 1H), 4.83 (br. s, 1H), 4.61 (br. m, 1H), 3.56 (br. m, 6H), 2.50 (br. m, 2H), 2.41 (br. m, 2H), 2.31 (d, 2H, J=9 Hz), 2.01 (br. m, 6H), 1.86 (br. m, 3H), 1.62 (br. m, 9H), 1.46 (br. m, 16H), 1.19 (br. m, 11H), 1.04 (s, 5H), 0.96 (d, 5H, J=6 Hz), 0.85 (q, 10H), 0.70 (s, 3H).

### BX. Compound SA155: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

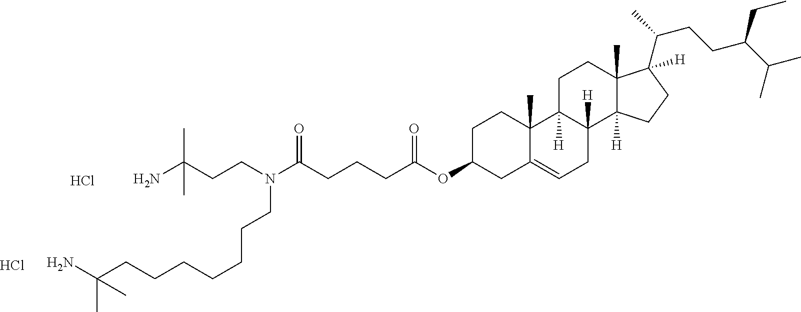

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate

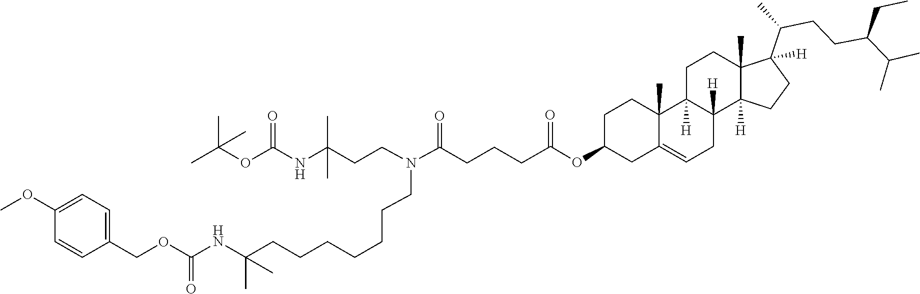

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.100 g, 0.189 mmol), tert-butyl N-(4-{[8-({[(4-methoxyphenyl)methoxy]carbonyl}amino)-8-methylnonyl]amino}-2-methylbutan-2-yl)carbamate (0.099 g, 0.19 mmol), and DMAP (0.046 g, 0.38 mmol) in DCM (2.0 mL) was added 1-ethyl-3-(3-dimethylaminopropyl)carbodiimide hydrochloride (0.073 g, 0.38 mmol). The reaction mixture was stirred at room temperature and was monitored by LCMS. At 15 hours, the reaction mixture was diluted with DCM (15 mL) and washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.121 g, 0.117 mmol, 62.0%) as a clear oil. UPLC/ELSD: RT=3.77 min. MS (ES): m/z=1034.04 (M+H)⁺ for C₆₃H₁₀₅N₃O₈. ¹H NMR (300 MHz, CDCl₃) δ 7.33-7.27 (m, 2H), 6.92-6.84 (m, 2H), 5.42-5.31 (m, 1H), 4.97 (s, 2H), 4.77-4.30 (m, 3H), 3.81 (s, 3H), 3.36-3.14 (m, 4H), 2.43-2.24 (m, 6H), 2.16-0.76 (m, 73H), 1.01 (s, 3H), 0.92 (d, J=6.5 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride

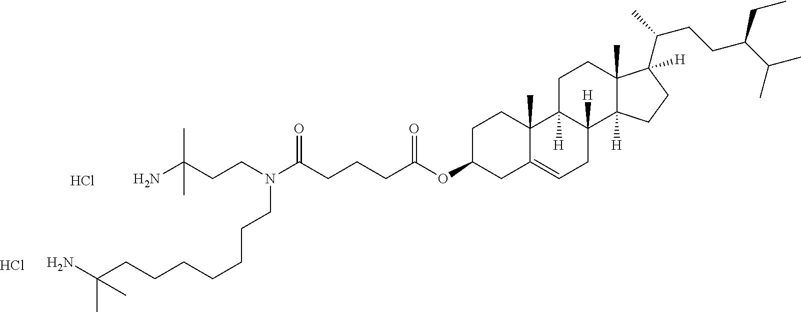

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-((tert-butoxycarbonyl)amino)-3-methylbutyl)(8-((((4-methoxybenzyl)oxy)carbonyl)amino)-8-methylnonyl)amino)-5-oxopentanoate (0.116 g, 0.112 mmol) in DCM (2.0 mL) was added 4 N HCl in dioxane (0.20 mL). The reaction mixture was stirred at room temperature and was monitored by LCMS. At 17 hours, 4 N HCl in dioxane (0.10 mL) was added. At 22 hours, MTBE (20 mL) was added, and the reaction mixture was held at 4° C. overnight. The suspension was centrifuged (10,000×g for 30 min at 4° C.). The supernatant was decanted. The solids were suspended in MTBE and concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-methylbutyl)(8-amino-8-methylnonyl)amino)-5-oxopentanoate dihydrochloride (0.073 g, 0.080 mmol, 71.4%) as a white solid. UPLC/ELSD: RT=2.38 min. MS (ES): m/z=385.65 (M+2H)²⁺ for C₄₉H₈₉N₃O₃. ¹H NMR (300 MHz, MeOD) δ 5.43-5.32 (m, 1H), 4.62-4.47 (m, 1H), 3.50-3.33 (m, 4H), 2.51-2.23 (m, 6H), 2.13-1.77 (m, 9H), 1.76-0.77 (m, 43H), 1.37 (s, 6H), 1.33 (s, 6H), 1.05 (s, 3H), 0.96 (d, J=6.4 Hz, 3H), 0.75-0.70 (m, 3H).

### BY. Compound SA156: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride

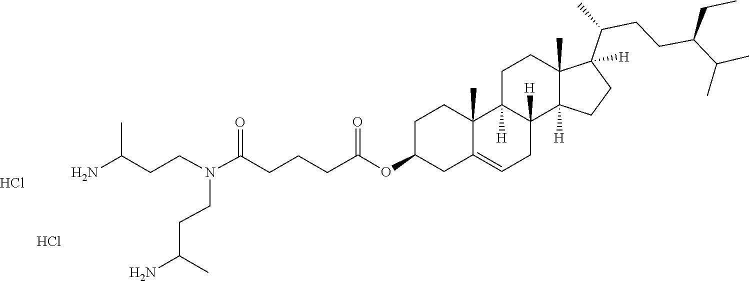

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-((tert-butoxycarbonyl)amino)butyl)amino)-5-oxopentanoate

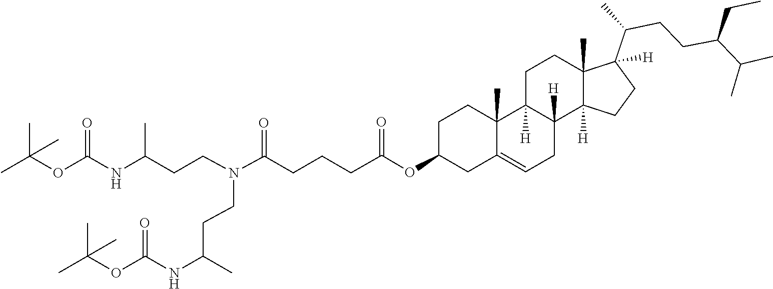

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.100 g, 0.189 mmol), tert-butyl N-[4-({3-[(tert-butoxycarbonyl)amino]butyl}amino)butan-2-yl]carbamate (0.075 g, 0.21 mmol), and DMAP (0.051 g, 0.42 mmol) in DCM (2.0 mL) was added 1-ethyl-3-(3-dimethylaminopropyl)carbodiimide hydrochloride (0.073 g, 0.38 mmol). The reaction mixture was stirred at room temperature and was monitored by LCMS. At 17 hours, the reaction mixture was diluted with DCM (10 mL), then washed with 5% aq. NaHCO₃soln. The aqueous was extracted with DCM (10 mL). The combined organics were passed through a hydrophobic frit, dried over Na₂SO₄, and concentrated. The crude material was purified via silica gel chromatography (20-65% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-((tert-butoxycarbonyl)amino)butyl)amino)-5-oxopentanoate (0.097 g, 0.11 mmol, 58.9%) as a white foam. UPLC/ELSD: RT=3.57 min. MS (ES): m/z=871.11 (M+H)⁺ for C₅₂H₉₁N₃O₇. ¹H NMR (300 MHz, CDCl₃) δ 5.48-5.30 (m, 1H), 4.77-4.42 (m, 3H), 3.79-3.07 (m, 6H), 2.52-2.19 (m, 6H), 2.14-0.76 (m, 66H), 1.01 (s, 3H), 0.92 (d, J=6.4 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride

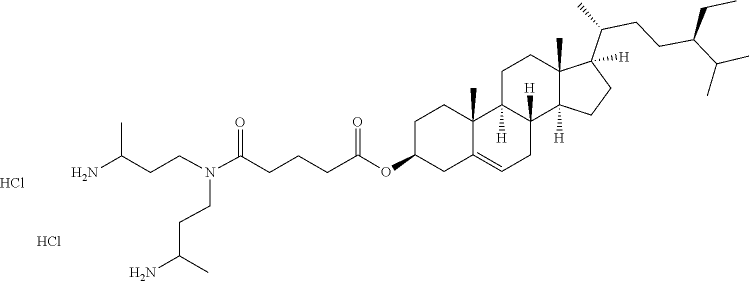

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-((tert-butoxycarbonyl)amino)butyl)amino)-5-oxopentanoate (0.086 g, 0.099 mmol) in DCM (1.8 mL) was added 4 N HCl in dioxane (0.25 mL). The reaction mixture was stirred at room temperature and was monitored by LCMS. At 16 hours, MTBE (20 mL) was added, and the reaction mixture was centrifuged (10,000×g for 15 min at 4° C.). The supernatant was drawn off, and the solids rinsed sparingly with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-(bis(3-aminobutyl)amino)-5-oxopentanoate dihydrochloride (0.066 g, 0.082 mmol, 82.8%) as a white solid. UPLC/ELSD: RT=2.22 min. MS (ES): m/z=670.59 (M+H)⁺ for C₄₂H₇₅N₃O₃. ¹H NMR (300 MHz, MeOD) δ 5.43-5.31 (m, 1H), 4.62-4.48 (m, 1H), 3.73-3.33 (m, 5H), 3.24-3.11 (m, 1H), 2.60-2.23 (m, 6H), 2.11-0.76 (m, 42H), 1.37 (d, J=6.6 Hz, 3H), 1.33 (d, J=6.6 Hz, 3H), 1.05 (s, 3H), 0.96 (d, J=6.4 Hz, 3H), 0.73 (s, 3H).

### BZ. Compound SA157: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

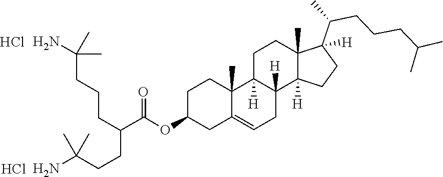

### Step 1: 4-((tert-butoxycarbonyl)amino)-4-methylpentyl 4-methylbenzenesulfonate

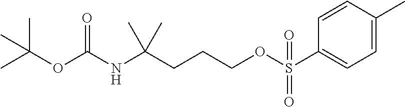 [SMILES: Cc1ccc(S(=O)(=O)OCCCC(C)(C)NC(=O)OC(C)(C)C)cc1]

To a solution of tert-butyl N-(5-hydroxy-2-methylpentan-2-yl)carbamate (2.50 g, 11.50 mmol) in dry DCM (30 mL) set stirring under nitrogen, was added triethylamine (8.02 mL, 57.52 mmol), dimethylaminopyridine (0.14 g, 1.15 mmol), and p-toluenesulfonyl chloride (4.39 g, 23.01 mmol). The solution was allowed to stir at room temperature for 6 hours, over which it turned a dark red color. The mixture was then further diluted with DCM, washed with water (1×30 mL), saturated aqueous sodium bicarbonate (1×30 mL) and brine (1×30 mL), dried over sodium sulfate, filtered, and concentrated to a dark brown oil. The oil was taken up in DCM and purified on silica in DCM with a 0-20% (80:19:1 DCM/MeOH/NH₄OH) gradient. Product-containing fractions were pooled and concentrated to give 4-((tert-butoxycarbonyl)amino)-4-methylpentyl 4-methylbenzenesulfonate as a light brown oil (3.55 g, 9.64 mmol, 83.0%). UPLC/ELSD: RT: 1.20 min. MS (ES): m/z (MH⁺) 372.4 for C₁₈H₂₉NO₅S. ¹H NMR (300 MHz, CDCl₃) δ 7.78 (d, 2H, J=9 Hz), 7.36 (d, 2H, J=9 Hz), 4.37 (br. s, 1H), 4.00 (br. m, 2H), 2.45 (s, 3H), 1.64 (br. s, 4H), 1.40 (s, 10H), 1.20 (s, 6H).

### Step 2: tert-butyl (5-((3-((tert-butoxycarbonyl)amino)-3-methylbutyl)amino)-2-methylpentan-2-yl)carbamate

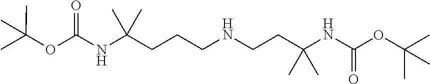 [SMILES: CC(C)(CCCNCCC(C)(C)NC(=O)OC(C)(C)C)NC(=O)OC(C)(C)C]

To a solution of tert-butyl N-[2-methyl-4-(2-nitrobenzenesulfonamido)butan-2-yl]carbamate (0.91 g, 2.34 mmol) in dry DMF (20 mL) stirring at room temperature under nitrogen, was added tert-butyl N-{2-methyl-5-[(4-methylbenzenesulfonyl)oxy]pentan-2-yl}carbamate (0.87 g, 2.34 mmol) and potassium carbonate (1.97 g, 14.25 mmol). The solution was warmed to 40° C. and stirred overnight. The following morning, the reaction was not complete by LC-MS, so it was heated to 100° C. and allowed to stir for an additional 3 hours. Then, the mixture was cooled to room temperature, and benzyl bromide (0.23 mL, 1.94 mmol) was added. The solution was stirred for 4 hours at room temperature and then thiophenol (0.92 mL, 8.99 mmol) was added, followed by additional potassium carbonate (0.97 g, 7.01 mmol) and DMF (20 mL). The solution stirred at room temperature overnight. The next morning, salts were removed from the mixture by centrifugation, and the supernatant was concentrated to a residue. The residue was taken up in 40 mL DCM, washed with water (2×10 mL) and brine (2×10 mL), dried over potassium carbonate, filtered, and concentrated to an oil. The oil was taken up in DCM and purified on silica in DCM with a 0-50% (50:45:5 DCM/MeOH/NH₄OH) gradient. Product-containing fractions were pooled and concentrated to give tert-butyl (5-((3-((tert-butoxycarbonyl)amino)-3-methylbutyl)amino)-2-methylpentan-2-yl)carbamate as a colorless oil (0.49 g, 1.22 mmol, 52.24%). UPLC/ELSD: RT: 0.30 min. MS (ES): m/z (MH⁺) 402.4 for C₂₁H₄₃N₃O₄. ¹H NMR (300 MHz, CDCl₃) δ 5.88 (br. s, 1H), 4.69 (br. s, 1H), 2.61 (t, 2H), 2.52 (t, 2H), 1.61 (br. m, 4H), 1.35 (s, 21H), 1.18 (s, 12H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate

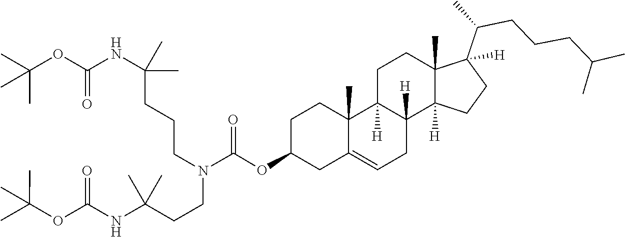

To a solution of tert-butyl (5-((3-((tert-butoxycarbonyl)amino)-3-methylbutyl)amino)-2-methylpentan-2-yl)carbamate (0.09 g, 0.22 mmol) in dry toluene (5 mL) set stirring under nitrogen was added triethylamine (0.07 mL, 0.09 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.12 g, 0.22 mmol) was added, and the solution was heated to 90° C. and allowed to proceed for 2 days. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate as a light yellow oil (0.16 g, 0.20 mmol, 89.9%). UPLC/ELSD: RT=3.67 min. MS (ES): m/z (MH⁺) 815.4 for C₄₉H₈₇N₃O₆. ¹H NMR (300 MHz, CDCl₃) δ 5.29 (br. s, 1H), 4.41 (br. m, 3H), 3.13 (br. m, 4H), 2.26 (br. m, 2H), 1.78 (br. m, 7H), 1.45 (br. m, 10H), 1.35 (s, 19H), 1.26 (br. m, 3H), 1.20 (s, 14H), 1.06 (br. m, 6H), 0.94 (s, 6H), 0.85 (d, 3H, J=6 Hz), 0.81 (d, 6H, J=6 Hz), 0.61 (s, 3H).

### Step 4: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride

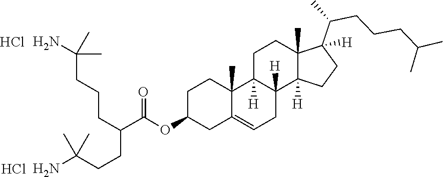

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-((tert-butoxycarbonyl)amino)-3-methylbutyl)(4-((tert-butoxycarbonyl)amino)-4-methylpentyl)carbamate (0.16 g, 0.20 mmol) in DCM (5 mL) set stirring under nitrogen was added hydrochloric acid (4 M in dioxanes, 0.50 mL, 2.01 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (3-amino-3-methylbutyl)(4-amino-4-methylpentyl)carbamate dihydrochloride as a white solid (0.09 g, 0.12 mmol, 60.2%). UPLC/ELSD: RT=1.90 min. MS (ES): m/z (MH⁺) 614.3 for C₃₉H₇₃Cl₂N₃O₂. ¹H NMR (300 MHz, MeOD) δ 5.41 (br. s, 1H), 4.45 (br. m, 1H), 3.33 (br. m, 6H), 2.37 (br. m, 2H), 1.93 (br. m, 7H), 1.60 (br. m, 11H), 1.37 (s, 15H), 1.18 (br. m, 6H), 1.08 (s, 5H), 0.98 (d, 3H, J=6 Hz), 0.89 (d, 7H, J=6 Hz), 0.75 (s, 3H).

### CA. Compound SA158: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride

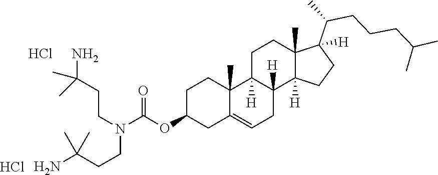

### Step 1: di-tert-butyl (azanediylbis(2-methylbutane-4,2-diyl))dicarbamate

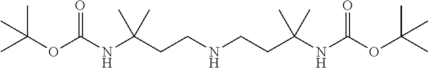 [SMILES: CC(C)(CCNCCC(C)(C)NC(=O)OC(C)(C)C)NC(=O)OC(C)(C)C]

Both tert-butyl (4-amino-2-methylbutan-2-yl)carbamate (0.50 g, 2.36 mmol) and tert-butyl (2-methyl-4-oxobutan-2-yl)carbamate (0.50 g, 2.36 mmol) were dissolve in 10 mL dry methanol and set stirring under nitrogen at room temperature. After two hours, sodium triacetoxyborohydride (1.25 g, 5.90 mmol) was added, and the reaction was allowed to continue stirring at room temperature overnight. The following morning, the amber reaction mixture was quenched with a few drops of water, concentrated to an orange oil, and taken back up in DCM. It was then washed with saturated sodium bicarbonate (1×15 mL) and brine (1×15 mL), dried over sodium sulfate, filtered, and concentrated to a yellow oil. The oil was resuspended in DCM and purified on silica in DCM with a 0-40% (50:45:5 DCM/MeOH/aqueous NH₄OH) gradient. Product-containing fractions were pooled and concentrated to give di-tert-butyl (azanediylbis(2-methylbutane-4,2-diyl))dicarbamate as a yellow oil (0.53 g, 1.37 mmol, 58.2%). UPLC/ELSD: RT=0.38 min. MS (ES): m/z (MH⁺) 388.6 for C₂₀H₄₁N₃O₄. ¹H NMR (300 MHz, CDCl₃) δ 5.56 (br. s, 2H), 2.67 (t, 4H), 1.77 (br. m, 5H), 1.62 (br. s, 4H), 1.44 (s, 18H), 1.30 (s, 12H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate

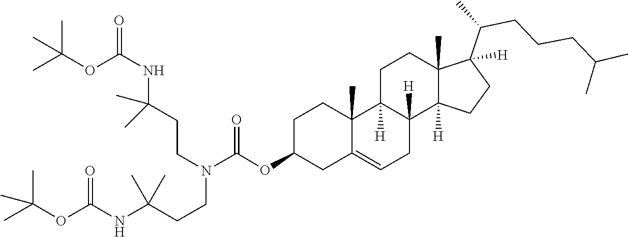

To a solution di-tert-butyl (azanediylbis(2-methylbutane-4,2-diyl))dicarbamate (0.10 g, 0.26 mmol) in dry toluene (5 mL) set stirring under nitrogen was added triethylamine (0.11 mL, 0.77 mmol). Then, (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl (4-nitrophenyl) carbonate (0.14 g, 0.26 mmol) was added, and the solution was heated to 90° C. and allowed to proceed for overnight. The following day, the reaction was not complete, so an additional 3 equivalents of triethylamine was added, and the reaction was allowed to proceed at 90° C. for an additional 24 hours. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate as a light yellow oil (0.15 g, 0.19 mmol, 72.2%). UPLC/ELSD: RT=3.41 min. MS (ES): m/z (MH⁺) 801.4 for C₄₈H₈₅N₃O₆. ¹H NMR (300 MHz, CDCl₃) δ 5.29 (br. s, 1H), 4.57 (br. s, 1H), 4.44 (br. m, 2H), 3.15 (br. m, 4H), 2.26 (m, 2H), 1.80 (m, 9H), 1.42 (br. m, 7H), 1.35 (s, 19H), 1.27 (m, 3H), 1.21 (s, 13H), 1.05 (br. m, 8H), 0.95 (s, 6H), 0.85 (d, 4H, J=6 Hz), 0.80 (d, 6H, J=6 Hz), 0.60 (s, 3H).

### Step 3: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride

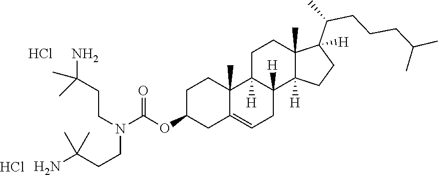

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate (0.15 g, 0.19 mmol) in DCM (3 mL) set stirring under nitrogen was added hydrochloric acid (4 M in dioxanes, 0.47 mL, 1.86 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl bis(3-amino-3-methylbutyl)carbamate dihydrochloride as a white solid (0.10 g, 0.14 mmol, 74.1%). UPLC/ELSD: RT=1.78 min. MS (ES): m/z (MH⁺) 601.3 for C₃₈H₇₁Cl₂N₃O₂. ¹H NMR (300 MHz, MeOD) δ 5.42 (br. s, 1H), 4.45 (br. m, 1H), 3.40 (m, 6H), 2.41 (d, 2H, J=6 Hz), 1.94 (br. m, 10H), 1.56 (br. m, 7H), 1.41 (s, 16H), 1.17 (br. m, 7H), 1.06 (s, 6H), 0.98 (d, 4H, J=6 Hz), 0.90 (d, 6H, J=6 Hz), 0.75 (s, 4H).

### CB. Compound SA159: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride

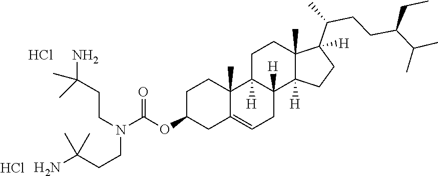

Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate

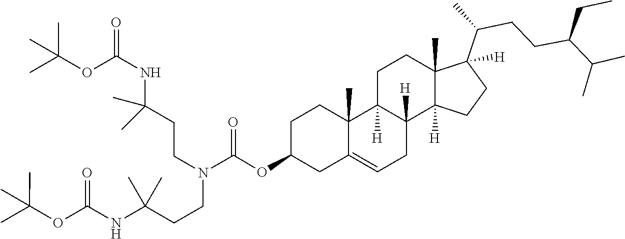

To a solution of di-tert-butyl (azanediylbis(2-methylbutane-4,2-diyl))dicarbamate (0.10 g, 0.26 mmol) in dry toluene (5 mL) set stirring under nitrogen was added triethylamine (0.11 mL, 0.77 mmol). Then, (1R,3aS,3bS,7S,9aR,9bS,11aR)-1-[(2R,5R)-5-ethyl-6-methylheptan-2-yl]-9a,11a-dimethyl-1H,2H,3H,3aH,3bH,4H,6H,7H,8H,9H,9bH,101H,11H-cyclopenta[a]phenanthren-7-yl 4-nitrophenyl carbonate (0.15 g, 0.26 mmol) was added, and the solution was heated to 90° C. and allowed to proceed for overnight. The following day, the reaction was not complete, so an additional 3 equivalents of triethylamine was added, and the reaction was allowed to proceed at 90° C. for an additional 24 hours. Then, the reaction mixture was allowed to cool to room temperature, diluted with toluene, washed with water (3×20 mL), dried over sodium sulfate, filtered, and concentrated to an oil. The oil was taken up in DCM and purified via silica gel chromatography in hexanes with a 0-50% EtOAc gradient. Fractions containing product were combined and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate as a light yellow oil (0.17 g, 0.20 mmol, 78.6%). UPLC/ELSD: RT=3.55 min. MS (ES): m/z (MH⁺) 823.4 for C₅₀H₈₉N₃O₆. ¹H NMR (300 MHz, CDCl₃) δ 5.41 (br. s, 1H), 4.53 (br. m, 3H), 3.25 (br. m, 4H), 2.40 (m, 2H), 1.90 (br. m, 9H), 1.58 (m, 7H), 1.45 (s, 18H), 1.30 (s, 13H), 1.18 (br. m, 7H), 1.04 (s, 5H), 0.95 (d, 5H, J=6 Hz), 0.83 (q, 9H), 0.70 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride

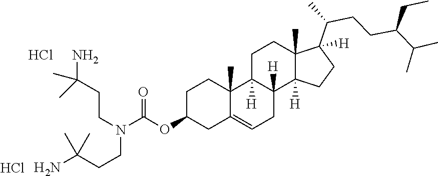

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-((tert-butoxycarbonyl)amino)-3-methylbutyl)carbamate (0.17 g, 0.20 mmol) in DCM (3 mL) set stirring under nitrogen was added hydrochloric acid (4 M in dioxanes, 0.51 mL, 2.03 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl bis(3-amino-3-methylbutyl)carbamate dihydrochloride as a white solid (0.12 g, 0.16 mmol, 77.4%). UPLC/ELSD: RT=1.94 min. MS (ES): m/z (MH⁺) 629.3 for C₄₀H₇₅Cl₂N₃O₂. ¹H NMR (300 MHz, MeOD) δ 5.42 (br. s, 1H), 4.45 (br. m, 1H), 3.38 (br. m, 6H), 2.41 (d, 2H, J=6 Hz), 1.94 (br. m, 9H), 1.56 (br. m, 8H), 1.41 (s, 15H), 1.22 (br. m, 6H), 1.09 (s, 6H), 0.99 (d, 4H, J=6 Hz), 0.87 (q, 9H), 0.75 (s, 4H).

### CC. Compound SA160: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

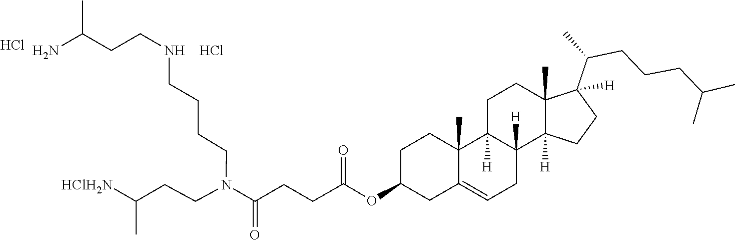

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate

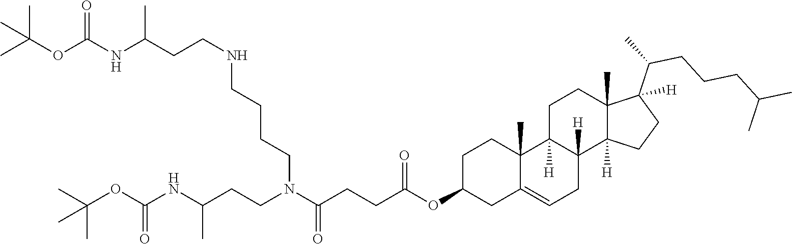

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.15 g, 0.31 mmol) in dry DCM (10 mL) stirring under nitrogen was added di-tert-butyl ((butane-1,4-diylbis(azanediyl))bis(butane-4,2-diyl))dicarbamate (0.33 g, 0.76 mmol), dimethylaminopyridine (0.08 g, 0.61 mmol), and 1-ethyl-3-(3-dimethylaminopropyl)carbodiimide hydrochloride (0.12 g, 0.61 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate as a light yellow oil (0.08 g, 0.09 mmol, 30.2%). UPLC/ELSD: RT: 2.49 min. MS (ES): m/z (MH⁺) 900.4 for C₅₃H₉₄N₄O₇. ¹H NMR (300 MHz, CDCl₃) δ 5.37 (br. s, 1H), 4.79 (br. m, 1H), 4.62 (br. m, 2H), 3.71 (br. m, 3H), 3.29 (m, 5H), 2.64 (br. m, 8H), 2.35 (t, 3H), 2.24 (s, 2H), 2.00 (br. m, 6H), 1.62 (br. m, 14H), 1.45 (s, 20H), 1.27 (br. m, 7H), 1.15 (m, 12H), 1.03 (s, 5H), 0.94 (d, 4H, J=6 Hz), 0.90 (d, 6H, J=6 Hz), 0.69 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

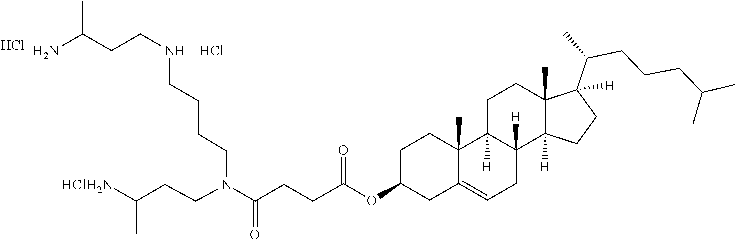

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate (0.08 g, 0.09 mmol) in isopropanol (3 mL) set stirring under nitrogen was added hydrochloric acid (5.5 M in isopropanol, 0.19 mL, 0.92 mmol) dropwise. The solution was allowed to stir at room temperature overnight. The following morning, acetonitrile (25 mL) was added to the mixture, which was cooled to 0° C. and allowed to stir for 30 minutes. The solution was then filtered and washed with 3:1 acetonitrile/isopropanol. The resulting solid was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride as a white solid (0.05 g, 0.06 mmol, 60.0%). UPLC/ELSD: RT=1.36 min. MS (ES): m/z (MH⁺) 700.3 for C₄₃H₈₁C₃N₄O₃. ¹H NMR (300 MHz, MeOD) δ 5.28 (br. s, 1H), 4.41 (br. m, 1H), 3.85 (m, 1H), 3.39 (br. m, 5H), 3.20 (s, 2H), 3.02 (br. m, 5H), 2.60 (br. m, 4H), 2.23 (br. m, 4H), 1.92 (s, 5H), 1.71 (br. m, 9H), 1.43 (br. m, 8H), 1.28 (m, 12H), 1.06 (d, 11H, J=6 Hz), 0.95 (s, 6H), 0.86 (d, 4H, J=6 Hz), 0.80 (d, 6H, J=6 Hz), 0.63 (s, 3H).

### CD. Compound SA161: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

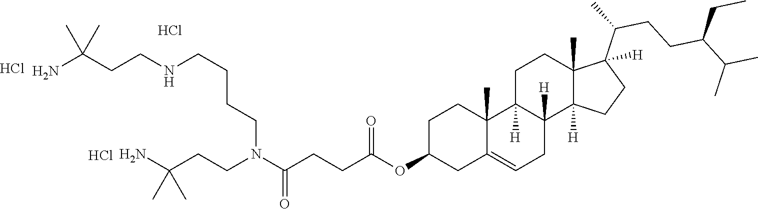

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate

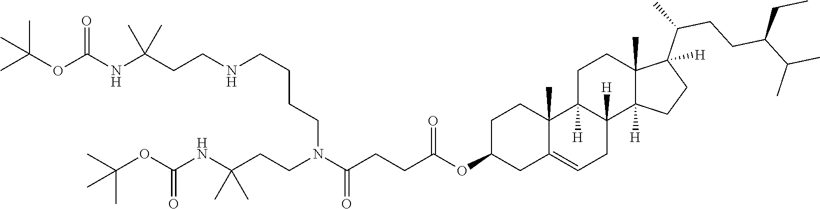

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.22 g, 0.48 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate as a light yellow oil (0.03 g, 0.03 mmol, 16.3%). UPLC/ELSD: RT: 2.77 min. MS (ES): m/z (MH⁺) 956.4 for C₅₇H₁₀₂N₄O₇. ¹H NMR (300 MHz, CDCl₃) δ 5.35 (br. s, 1H), 4.60 (br. m, 1H), 3.25 (br. m, 5H), 3.00 (br. m, 2H), 2.60 (br. m, 6H), 2.34 (d, 3H, J=6 Hz), 2.23 (m, 4H), 1.99 (br. m, 3H), 1.87 (br. m, 4H), 1.63 (br. m, 12H), 1.42 (s, 18H), 1.28 (d, 15H, J=6 Hz), 1.12 (br. m, 7H), 1.02 (s, 5H), 0.93 (d, 5H, J=6 Hz), 0.83 (q, 8H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

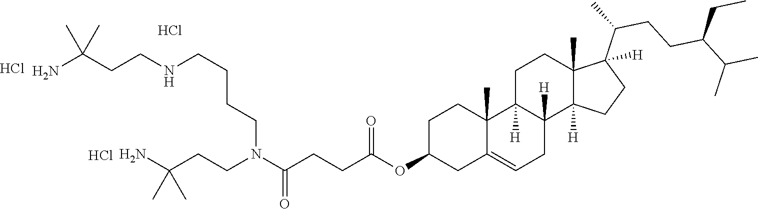

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate (0.03 g, 0.03 mmol) in DCM (1 mL) set stirring under nitrogen was added hydrochloric acid (4 M in dioxanes, 0.08 mL, 0.31 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride as a white solid (0.03 g, 0.03 mmol, 89.0%). UPLC/ELSD: RT=1.69 min. MS (ES): m/z (MH⁺) 756.3 for C₄₇H₈₉Cl₃N₄O₃. ¹H NMR (300 MHz, MeOD) δ 5.41 (br. s, 1H), 4.56 (br. m, 1H), 3.49 (br. m, 5H), 3.33 (br. s, 2H), 3.18 (br. m, 5H), 2.94 (m, 1H), 2.65 (br. m, 4H), 2.33 (d, 2H, J=6 Hz), 2.15 (br. m, 5H), 1.84 (br. m, 8H), 1.63 (br. m, 9H), 1.43 (t, 15H), 1.25 (br. m, 10H), 1.07 (s, 5H), 0.97 (d, 5H, J=6 Hz), 0.89 (q, 9H), 0.75 (s, 3H).

### CE. Compound SA162: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

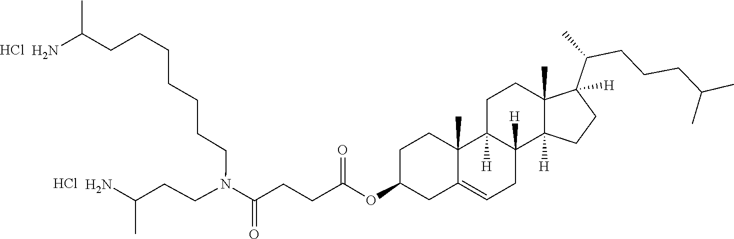

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate

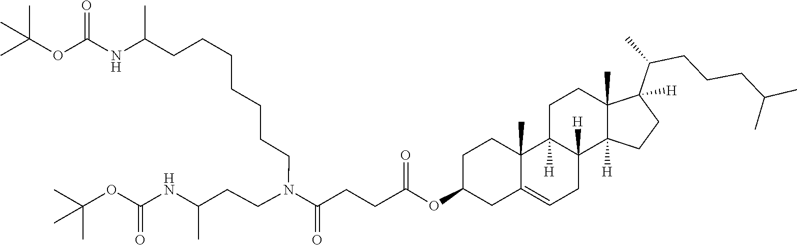

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate as a light yellow oil (0.11 g, 0.12 mmol, 67.6%). UPLC/ELSD: RT: 3.29 min. MS (ES): m/z (MH⁺) 899.4 for C₅₄H₉₅N₃O₇. ¹H NMR (300 MHz, CDCl₃) δ 5.30 (br. s, 1H), 4.53 (br. m, 2H), 4.34 (br. s, 1H), 3.54 (br. m, 2H), 3.14 (br. m, 4H), 2.56 (m, 4H), 2.26 (d, 2H, J=9 Hz), 1.85 (br. m, 5H), 1.49 (br. m, 9H), 1.36 (s, 19H), 1.24 (br. m, 13H), 1.02 (m, 13H), 0.94 (s, 5H), 0.85 (d, 4H, J=6 Hz), 0.81 (d, 6H, J=6 Hz), 0.60 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

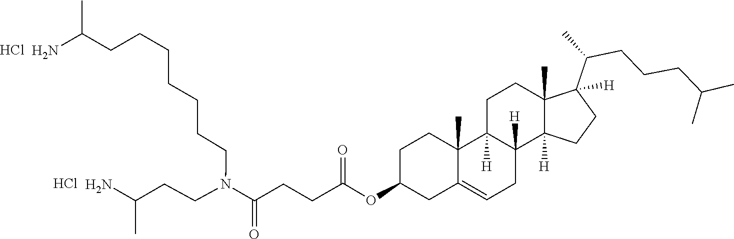

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate (0.11 g, 0.12 mmol) in DCM (3 mL) set stirring under nitrogen was added hydrochloric acid (4 N in dioxanes, 0.30 mL, 1.18 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride as a white solid (0.08 g, 0.10 mmol, 86.1%). UPLC/ELSD: RT=1.75 min. MS (ES): m/z (MH⁺) 699.3 for C₄₄H₈₁C₂N₃O₃. ¹H NMR (300 MHz, CDCl₃) δ 8.40 (m, 6H), 5.39 (br. s, 1H), 4.64 (br. m, 1H), 3.39 (br. m, 5H), 2.66 (s, 4H), 2.35 (d, 2H, J=6 Hz), 2.04 (br. m, 4H), 1.86 (m, 3H), 1.58 (br. m, 9H), 1.46 (br. m, 21H), 1.12 (br. m, 7H), 1.03 (s, 6H), 0.92 (d, 4H, J=6 Hz), 0.88 (d, 7H, J=6 Hz), 0.69 (s, 3H).

### CF. Compound SA163: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

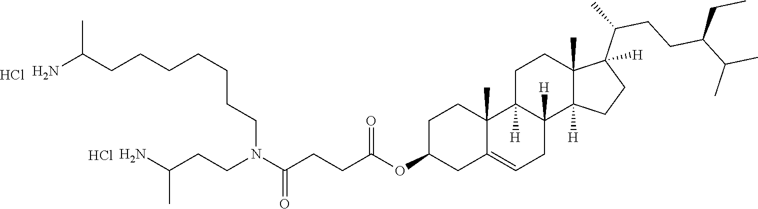

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate

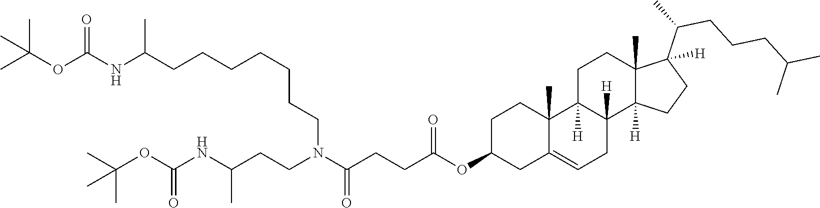

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-[4-({8-[(tert-butoxycarbonyl)amino]nonyl}amino)butan-2-yl]carbamate (0.08 g, 0.19 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate as a light yellow oil (0.16 g, 0.17 mmol, 86.7%). UPLC/ELSD: RT: 3.65 min. MS (ES): m/z (MH⁺) 927.4 for C₅₆H₉₉N₃O₇. ¹H NMR (300 MHz, CDCl₃) δ 5.28 (br. s, 1H), 4.56 (br. m, 3H), 3.54 (br. m, 3H), 3.16 (br. m, 3H), 2.57 (m, 4H), 2.26 (d, 2H, J=3 Hz), 1.76 (br. m, 5H), 1.52 (br. m, 9H), 1.36 (s, 20H), 1.23 (br. m, 15H), 1.08 (br. m, 12H), 0.94 (s, 6H), 0.86 (d, 5H, J=6 Hz), 0.75 (q, 9H), 0.60 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride

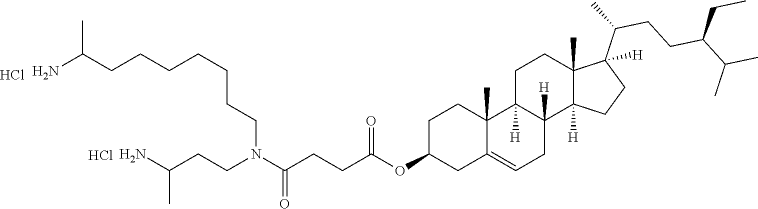

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)butyl)(8-((tert-butoxycarbonyl)amino)nonyl)amino)-4-oxobutanoate (0.16 g, 0.17 mmol) in DCM (3 mL) set stirring under nitrogen was added hydrochloric acid (4 M in dioxanes, 0.42 mL, 1.68 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(8-aminononyl)amino)-4-oxobutanoate dihydrochloride as a white solid (0.13 g, 0.13 mmol, 79.3%). UPLC/ELSD: RT=2.09 min. MS (ES): m/z (MH⁺) 728.3 for C₄₆H₈₅Cl₂N₃O₃. ¹H NMR (300 MHz, MeOD) δ 5.40 (br. s, 1H), 4.54 (br. m, 1H), 3.69 (br.m, 1H), 3.33 (s, 9H), 2.65 (br. m, 4H), 2.32 (d, 2H, J=6 Hz), 1.92 (br. m, 7H), 1.63 (br. m, 11H), 1.43 (br. m, 11H), 1.32 (t, 8H), 1.20 (br. m, 7H), 1.07 (s, 5H), 0.99 (d, 5H, J=6 Hz), 0.87 (q, 9H), 0.75 (s, 3H).

### CG. Compound SA164: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

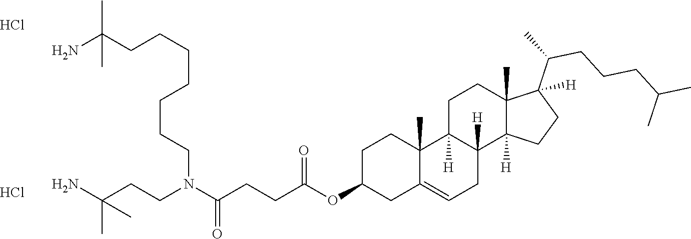

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate

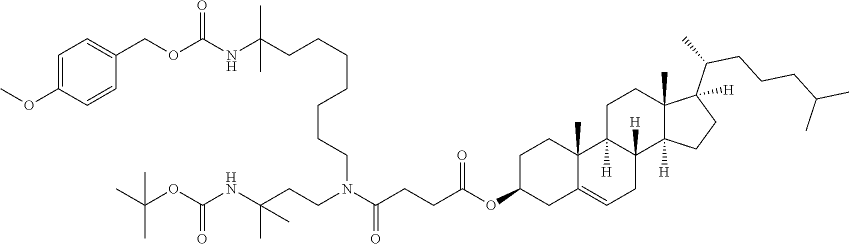

To a stirred solution of cholesteryl hemisuccinate (0.100 g, 0.205 mmol), tert-butyl N-(4-{[8-({[(4-methoxyphenyl)methoxy]carbonyl}amino)-8-methylnonyl]amino}-2-methylbutan-2-yl)carbamate (0.107 g, 0.205 mmol), and DMAP (cat.) in DCM (2 mL) cooled to 0° C. was added 1-ethyl-3-(3-dimethylaminopropyl)carbodiimide hydrochloride (0.079 g, 0.411 mmol). The reaction mixture was stirred at room temperature and was monitored by LCMS. At 16 hours, DMAP (0.050 g, 0.41 mmol) and 1-ethyl-3-(3-dimethylaminopropyl)carbodiimide hydrochloride (40 mg) were added. At 43 hours, 1-ethyl-3-(3-dimethylaminopropyl)carbodiimide hydrochloride (65 mg) was added. At 64 hours, the reaction mixture was diluted with DCM (15 mL), and then washed with 5% aq. NaHCO₃ soln. The aqueous was extracted with DCM (15 mL). The combined organics were washed with 5% aq. NaHCO₃ soln., passed through a hydrophobic frit, dried over Na₂SO₄, and concentrated. The crude material was purified via silica gel chromatography (0-50% EtOAc in hexanes) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.097 g, 0.098 mmol, 47.7%) as a clear oil. UPLC/ELSD: RT=3.68 min. MS (ES): m/z=990.87 (M+H)⁺ for C₆₀H₉₉N₃O₈. ¹H NMR (300 MHz, CDCl₃) δ 7.33-7.27 (m, 2H), 6.92-6.84 (m, 2H), 5.41-5.31 (m, 1H), 4.97 (s, 2H), 4.77-4.38 (m, 3H), 3.81 (s, 3H), 3.37-3.18 (m, 4H), 2.71-2.51 (m, 4H), 2.38-2.26 (m, 2H), 2.17-1.04 (m, 61H), 1.01 (s, 3H), 0.91 (d, J=5.9 Hz, 3H), 0.86 (d, J=6.6 Hz, 3H), 0.86 (d, J=6.6 Hz, 3H), 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

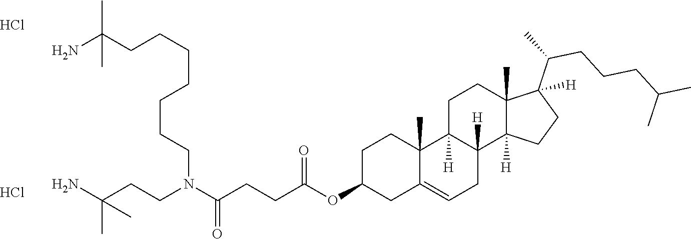

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.093 g, 0.094 mmol) in DCM (1.5 mL) was added 4 N HCl in dioxane (0.17 mL). The reaction mixture was stirred at room temperature and was monitored by LCMS. At 17 hours, 4 N HCl in dioxane (0.07 mL) was added. At 22 hours, MTBE (10 mL) was added, and the reaction mixture was held at 4° C. overnight. The reaction mixture was blown down under N₂ stream until gelatinous. Then, ice cold MTBE (10 mL) was added, and the suspension was centrifuged (10,000×g for 1 h at 4° C.). The supernatant was decanted. The solids were rinsed with cold MTBE, suspended in MTBE, and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride (0.058 g, 0.068 mmol, 72.8%) as a white solid. UPLC/ELSD: RT=2.23 min. MS (ES): m/z=364.70 (M+2H)²⁺ for C₄₆H₈₃N₃O₃. ¹H NMR (300 MHz, MeOD) δ 5.43-5.34 (m, 1H), 4.62-4.45 (m, 1H), 3.56-3.34 (m, 4H), 2.73-2.57 (m, 4H), 2.42-2.26 (m, 2H), 2.14-1.77 (m, 7H), 1.75-0.97 (m, 33H), 1.05 (s, 3H), 1.36 (s, 6H), 1.33 (s, 6H), 0.95 (d, J=6.5 Hz, 3H), 0.88 (d, J=6.5 Hz, 6H), 0.73 (s, 3H).

### CH. Compound SA165: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

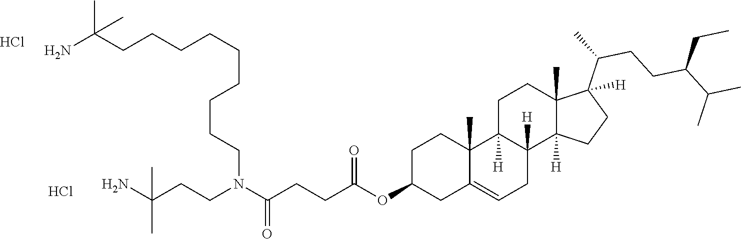

### Step 1: 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid

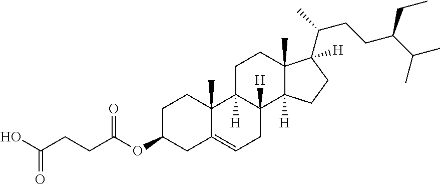

Sitosterol (3.00 g, 7.23 mmol) and succinic anhydride (0.941 g, 9.40 mmol) were combined in pyridine (6.0 mL). The reaction mixture was stirred at 80° C. and was monitored by TLC. At 19 hours, DMAP (cat.) added. At 89 hours, the reaction mixture was cooled to room temperature, diluted with DCM (100 mL), and washed with water. The organics were extracted with aq. 1 N NaOH (3×50 mL). A precipitate formed. The mixture was filtered. The solids were taken up in aq. 1 N HCl, and then extracted with DCM (3×50 mL). The organic extracts were washed with aq. 1 N HCl (2×) and water, passed through a hydrophobic frit, dried over Na₂SO₄, and concentrated. The residue was dissolved in DCM (5 mL) and hexanes (30 mL) was added while heating. Heat (hot water bath at 37° C.) was used to drive off solvent until solids formed. The solution was allowed to cool to room temperature, and was further cooled to 0° C. After 1.5 hours, white solids formed. The mixture was allowed to warm to room temperature, and solids were collected by vacuum filtration rinsing with cold 9:1 hexanes/DCM to afford 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.436 g, 0.847 mmol, 11.7%) as an off-white solid. ¹H NMR (300 MHz, CDCl₃) δ 5.42-5.30 (m, 1H), 4.71-4.56 (m, 1H), 2.72-2.55 (m, 4H), 2.36-2.23 (m, 2H), 2.09-1.75 (m, 5H), 1.73-0.75 (m, 31H), 1.02 (s, 3H), 0.92 (d, J=6.4 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate

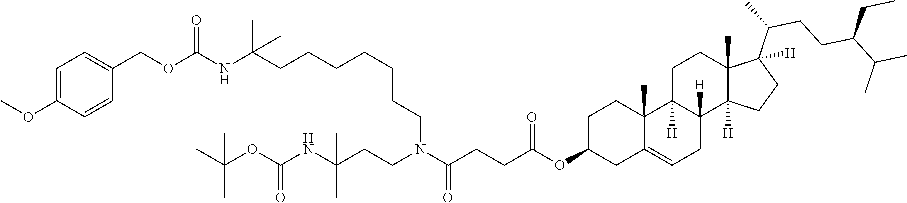

To a stirred solution of 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.100 g, 0.194 mmol), tert-butyl N-(4-{[8-({[(4-methoxyphenyl)methoxy]carbonyl}amino)-8-methylnonyl]amino}-2-methylbutan-2-yl)carbamate (0.111 g, 0.214 mmol), and DMAP (cat.) in DCM (2.0 mL) cooled to 0° C. was added 1-ethyl-3-(3-dimethylaminopropyl)carbodiimide hydrochloride (0.074 g, 0.39 mmol). The reaction was mixture stirred at room temperature and was monitored by LCMS. At 16 hours, DMAP (0.047 g, 0.39 mmol) was added, followed by 1-ethyl-3-(3-dimethylaminopropyl)carbodiimide hydrochloride (45 mg). At 43 hours, 1-ethyl-3-(3-dimethylaminopropyl)carbodiimide hydrochloride (65 mg) was added. At 64 hours, the reaction mixture was diluted with DCM (15 mL), and washed with 5% aq. NaHCO₃ soln. The aqueous was extracted with DCM (15 mL). The combined organics were washed with 5% aq. NaHCO₃ soln., passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.097 g, 0.095 mmol, 49.0%) as a clear oil. UPLC/ELSD: RT=3.74 min. MS (ES): m/z=1018.87 (M+H)⁺ for C₆₂H₁₀₃N₃O₈. ¹H NMR (300 MHz, CDCl₃) δ 7.34-7.26 (m, 2H), 6.92-6.83 (m, 2H), 5.39-5.32 (m, 1H), 4.97 (s, 2H), 4.80-4.36 (m, 3H), 3.80 (s, 3H), 3.39-3.18 (m, 4H), 2.69-2.52 (m, 4H), 2.35-2.23 (m, 2H), 2.12-0.77 (m, 71H), 1.01 (s, 3H), 0.92 (d, J=6.4 Hz, 3H), 0.67 (s, 3H).

### Step 3: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride

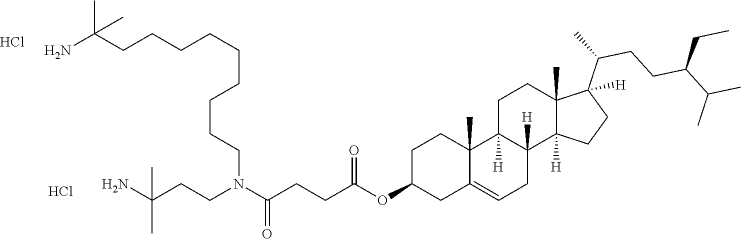

To a stirred solution (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(8-((((4-methoxybenzyl)oxy)carbonyl)amino)-8-methylnonyl)amino)-4-oxobutanoate (0.093 g, 0.091 mmol) in DCM (1.5 mL) was added 4 N HCl in dioxane (0.17 mL). The reaction mixture was stirred at room temperature and was monitored by LCMS. At 17 hours, 4 N HCl in dioxane (0.07 mL) was added. At 22 hours, MTBE (10 mL) added, and the reaction mixture was held at 4° C. overnight. The reaction mixture was blown down under a stream of N₂ until gelatinous. Cold MTBE (10 mL) was added, and the suspension was centrifuged (10,000×g for 1 h at 4° C.). The supernatant was decanted, the solids were rinsed with cold MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(8-amino-8-methylnonyl)amino)-4-oxobutanoate dihydrochloride (0.034 g, 0.039 mmol, 42.2%) as a white solid. UPLC/ELSD: RT=2.34 min. MS (ES): m/z=377.76 (M+2H)²⁺ for C₄₈H₈₇N₃O₃. ¹H NMR (300 MHz, MeOD) δ 5.46-5.32 (m, 1H), 4.65-4.43 (m, 1H), 3.54-3.34 (m, 4H), 2.74-2.50 (m, 4H), 2.45-2.21 (m, 2H), 2.13-1.79 (m, 7H), 1.77-0.78 (m, 43H), 1.36 (s, 6H), 1.32 (s, 6H), 1.05 (s, 3H), 0.96 (d, J=6.5 Hz, 3H), 0.73 (s, 3H).

### CI. Compound SA166: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

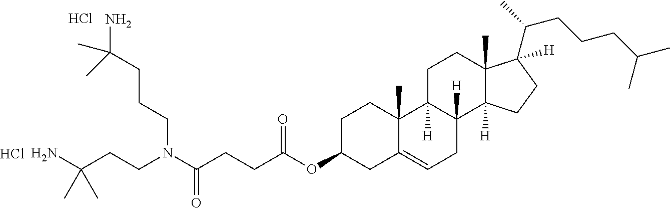

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate

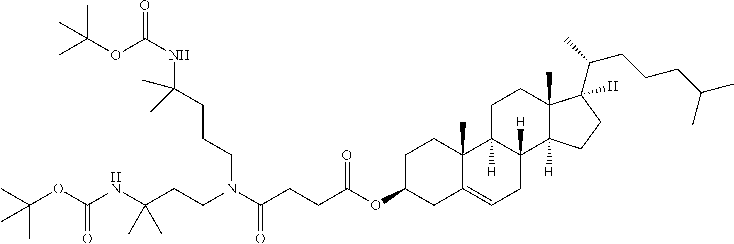

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.11 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate as a light yellow oil (0.17 g, 0.19 mmol, 85.6%). UPLC/ELSD: RT: 3.55 min. MS (ES): m/z (MH⁺) 871.4 for C₅₂H₉₁N₃O₇. ¹H NMR (300 MHz, CDCl₃) δ 5.37 (br. s, 1H), 4.62 (br. m, 3H), 3.27 (br. m, 4H), 2.64 (br. m, 4H), 2.35 (d, 2H, J=6 Hz), 2.01 (br. m, 3H), 1.85 (br. m, 4H), 1.56 (br. m, 11H), 1.44 (s, 17H), 1.35 (br. m, 3H), 1.30 (m, 13H), 1.14 (br. m, 6H), 1.03 (s, 5H), 0.95 (d, 3H, J=6 Hz), 0.90 (d, 6H, J=6 Hz), 0.70 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

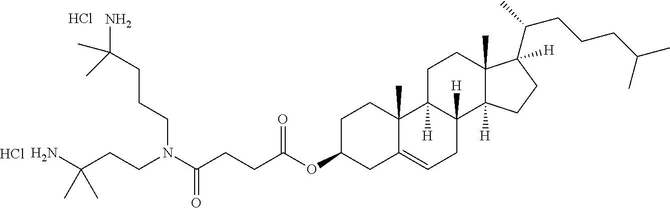

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate (0.17 g, 0.19 mmol) in DCM (5 mL) set stirring under nitrogen was added hydrochloric acid (4 M in dioxanes, 0.48 mL, 1.92 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride as a white solid (0.12 g, 0.14 mmol, 74.0%). UPLC/ELSD: RT=1.99 min. MS (ES): m/z (MH⁺) 671.3 for C₄₂H₇₇Cl₂N₃O₃. ¹H NMR (300 MHz, MeOD) δ 5.52 (s, 1H), 5.39 (br. s, 1H), 4.56 (br. m, 1H), 3.68 (s, 1H), 3.47 (br. m, 3H), 3.32 (br. s, 2H), 2.64 (br. m, 4H), 2.35 (br. m, 2H), 1.90 (br. m, 6H), 1.55 (br. m, 10H), 1.46 (s, 3H), 1.40 (br. s, 14H), 1.16 (br. m, 5H), 1.07 (s, 5H), 0.98 (d, 5H, J=6 Hz), 0.89 (d, 8H, J=6 Hz), 0.75 (s, 3H).

### CJ. Compound SA167: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

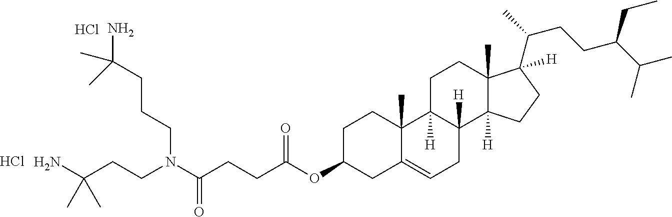

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate

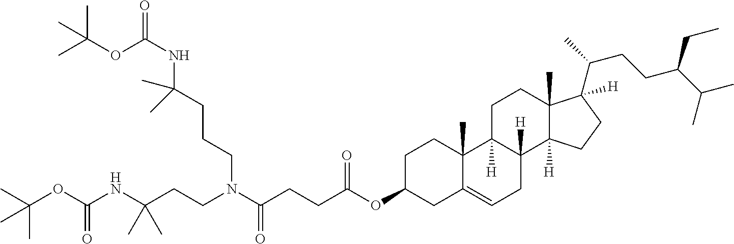

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.12 g, 0.22 mmol) in dry DCM (5 mL) stirring under nitrogen was added tert-butyl (5-((3-((tert-butoxycarbonyl)amino)-3-methylbutyl)amino)-2-methylpentan-2-yl)carbamate (0.09 g, 0.22 mmol), dimethylaminopyridine (0.06 g, 0.45 mmol), and 1-ethyl-3-(3-dimethylaminopropyl)carbodiimide hydrochloride (0.09 g, 0.45 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate as a light yellow oil (0.14 g, 0.15 mmol, 67.6%). UPLC/ELSD: RT: 3.64 min. MS (ES): m/z (MH⁺) 899.4 for C₅₄H₉₅N₃O₇. ¹H NMR (300 MHz, CDCl₃) δ 5.31 (br. s, 1H), 4.57 (br. m, 3H), 3.24 (br. m, 4H), 2.57 (br. m, 4H), 2.30 (d, 2H, J=6 Hz), 2.00 (br. m, 4H), 1.82 (br. m, 4H), 1.53 (br. m, 11H), 1.38 (s, 18H), 1.31 (br. m, 2H), 1.24 (m, 16H), 1.11 (br. m, 6H), 0.98 (s, 6H), 0.88 (d, 5H, J=6 Hz), 0.79 (q, 10H), 0.64 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride

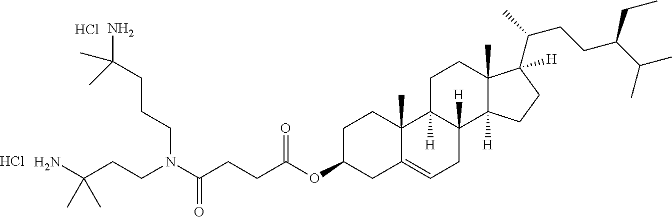

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-((tert-butoxycarbonyl)amino)-3-methylbutyl)(4-((tert-butoxycarbonyl)amino)-4-methylpentyl)amino)-4-oxobutanoate (0.14 g, 0.15 mmol) in DCM (5 mL) set stirring under nitrogen was added hydrochloric acid (4 M in dioxanes, 0.38 mL, 1.51 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-amino-3-methylbutyl)(4-amino-4-methylpentyl)amino)-4-oxobutanoate dihydrochloride as a white solid (0.10 g, 0.12 mmol, 80.3%). UPLC/ELSD: RT=2.18 min. MS (ES): m/z (MH⁺) 699.3 for C₄₄H₈₁Cl₂N₃O₃. ¹H NMR (300 MHz, MeOD) δ 5.41 (br. s, 1H), 4.53 (br. m, 1H), 3.46 (br. m, 3H), 3.32 (m, 5H), 2.65 (br. m, 4H), 2.33 (br. m, 2H), 1.91 (br. m, 7H), 1.64 (br. m, 11H), 1.45 (s, 3H), 1.40 (s, 13H), 1.20 (br. m, 7H), 1.07 (s, 5H), 0.99 (d, 4H, J=6 Hz), 0.87 (q, 9H), 0.75 (s, 4H).

### CK. Compound SA168: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride

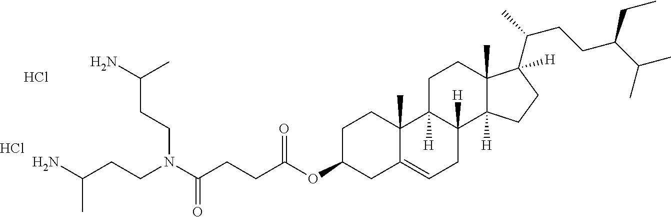

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate

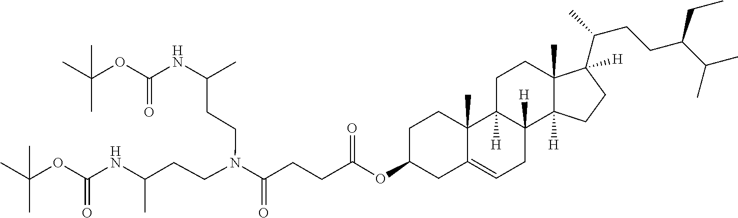

To a stirred solution of 4-4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.100 g, 0.194 mmol), tert-butyl N-[4-({3-[(tert-butoxycarbonyl)amino]butyl}amino)butan-2-yl]carbamate (0.077 g, 0.21 mmol), and DMAP (0.052 g, 0.43 mmol) in DCM (2.0 mL) was added 1-ethyl-3-(3-dimethylaminopropyl)carbodiimide hydrochloride (0.074 g, 0.39 mmol). The reaction mixture was stirred at room temperature and was monitored by LCMS. At 17 hours, the reaction mixture was diluted with DCM (15 mL), then washed with 5% aq. NaHCO₃ soln. The aqueous was extracted with DCM (15 mL). The combined organics were passed through a hydrophobic frit, dried over Na₂SO₄, and concentrated. The crude material was purified via silica gel chromatography (20-65% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate (0.129 g, 0.151 mmol, 77.6%) as a clear oil. UPLC/ELSD: RT=3.53 min. MS (ES): m/z=856.81 (M+H)⁺ for C₅₁H₈₉N₃O₇. ¹H NMR (300 MHz, CDCl₃) δ 5.41-5.32 (m, 1H), 4.70-4.35 (m, 3H), 3.78-3.08 (m, 6H), 2.74-2.46 (m, 4H), 2.38-2.23 (m, 2H), 2.10-0.75 (m, 64H), 1.01 (s, 3H), 0.92 (d, J=6.3 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride

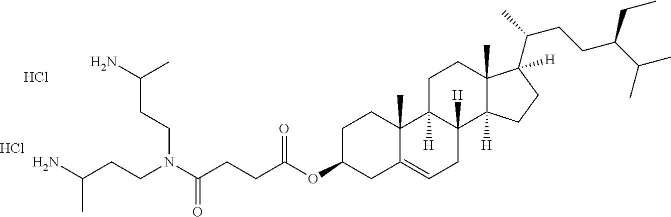

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)butyl)amino)-4-oxobutanoate (0.125 g, 0.146 mmol) in DCM (2.5 mL) was added 4 N HCl in dioxane (0.37 mL). The reaction mixture was stirred at room temperature and was monitored by LCMS. At 16 hours, MTBE (20 mL) was added, and the reaction mixture was centrifuged (10,000×g for 15 min at 4° C.). The supernatant was drawn off, and the solids rinsed sparingly with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-aminobutyl)amino)-4-oxobutanoate dihydrochloride (0.084 g, 0.11 mmol, 74.8%) as a white solid. UPLC/ELSD: RT=2.11 min. MS (ES): m/z=349.41 [(M+2H)+CH₃CN]²⁺ for C₄₁H₇₃N₃O₃. ¹H NMR (300 MHz, MeOD) δ 5.43-5.34 (m, 1H), 4.62-4.46 (m, 1H), 3.75-3.34 (m, 5H), 3.26-3.15 (m, 1H), 2.83-2.59 (m, 4H), 2.42-2.25 (m, 2H), 2.16-0.78 (m, 40H), 1.38 (d, J=6.6 Hz, 3H), 1.32 (d, J=6.5 Hz, 3H), 1.05 (s, 3H), 0.96 (d, J=6.4 Hz, 3H), 0.73 (s, 3H).

### CL. Compound SA169: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

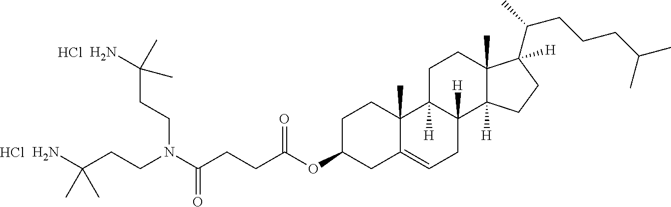

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate

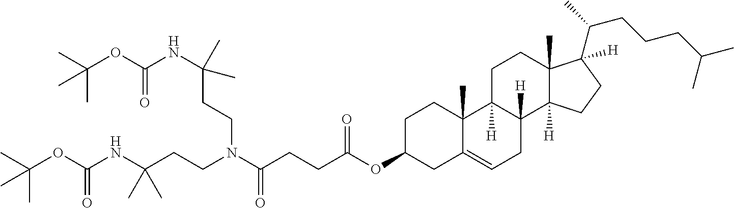

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate as a light yellow oil (0.22 g, 0.26 mmol, 100.0%). UPLC/ELSD: RT: 3.58 min. MS (ES): m/z (MH⁺) 857.4 for C₅₁H₈₉N₃O₇. ¹H NMR (300 MHz, CDCl₃) δ 5.07 (br. s, 1H), 4.55 (br. s, 1H), 4.32 (br. m, 1H), 4.20 (br. s, 1H), 3.04 (br. m, 4H), 2.33 (s, 4H), 2.05 (br. s, 2H), 1.65 (br. m, 9H), 1.28 (br. m, 7H), 1.14 (s, 19H), 1.00 (s, 15H), 0.83 (br. m, 7H), 0.74 (s, 6H), 0.65 (d, 4H, J=6 Hz), 0.60 (d, 6H, J=6 Hz), 0.39 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

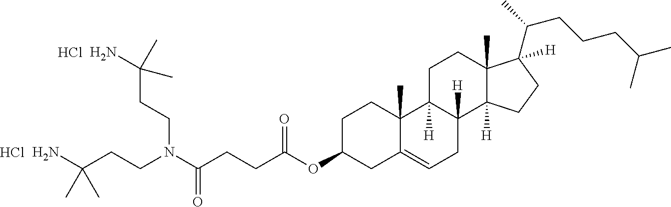

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate (0.22 g, 0.26 mmol) in DCM (5 mL) set stirring under nitrogen was added hydrochloric acid (4 M in dioxanes, 0.65 mL, 2.59 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride as a white solid (0.12 g, 0.16 mmol, 62.4%). UPLC/ELSD: RT=2.03 min. MS (ES): m/z (MH⁺) 657.3 for C₄₁H₇₅Cl₂N₃O₃. ¹H NMR (300 MHz, MeOD) δ 5.39 (br. s, 1H), 4.53 (br. m, 1H), 3.48 (br. m, 4H), 3.33 (br. s, 3H), 2.67 (br. m, 4H), 2.33 (br. m, 2H), 2.04 (br. m, 3H), 1.91 (br. m, 6H), 1.55 (br. m, 7H), 1.46 (s, 6H), 1.40 (s, 8H), 1.16 (br. m, 11H), 1.07 (s, 6H), 0.96 (d, 4H, J=6 Hz), 0.89 (d, 8H, J=6 Hz), 0.75 (s, 4H).

### CM. Compound SA170: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

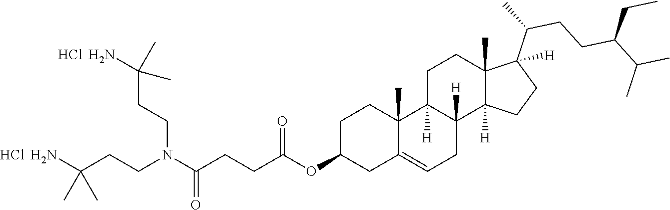

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate

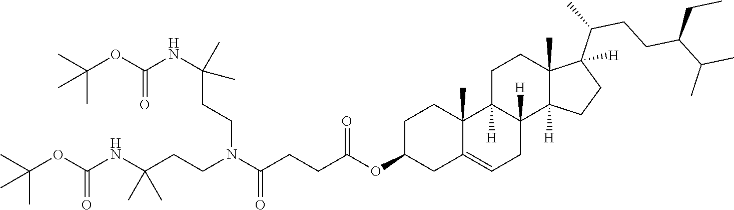

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate as a light yellow oil (0.18 g, 0.21 mmol, 80.2%). UPLC/ELSD: RT: 3.65 min. MS (ES): m/z (MH⁺) 885.4 for C₅₃H₉₃N₃O₇. ¹H NMR (300 MHz, CDCl₃) δ 5.06 (br. s, 1H), 4.54 (br. s, 1H), 4.33 (br. m, 1H), 4.21 (s, 1H), 3.03 (m, 4H), 2.32 (s, 4H), 2.02 (d, 2H, J=6 Hz), 1.63 (br. m, 9H), 1.29 (br. m, 7H), 1.14 (s, 19H), 0.99 (s, 15H), 0.84 (br. m, 6H), 0.73 (s, 5H), 0.65 (d, 5H, J=6 Hz), 0.54 (q, 9H), 0.39 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride

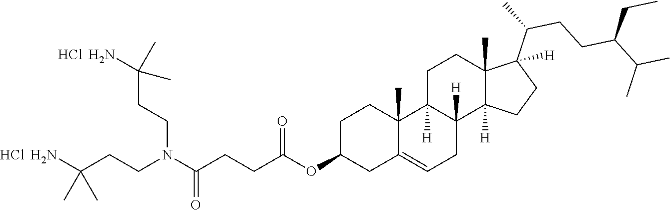

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-((tert-butoxycarbonyl)amino)-3-methylbutyl)amino)-4-oxobutanoate (0.18 g, 0.21 mmol) in DCM (5 mL) set stirring under nitrogen was added hydrochloric acid (4 M in dioxanes, 0.52 mL, 2.07 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-(bis(3-amino-3-methylbutyl)amino)-4-oxobutanoate dihydrochloride as a white solid (0.16 g, 0.19 mmol, 91.5%). UPLC/ELSD: RT=2.13 min. MS (ES): m/z (MH⁺) 685.3 for C₄₃H₇₉Cl₂N₃O₃. ¹H NMR (300 MHz, MeOD) δ 5.39 (br. s, 1H), 4.53 (br. m, 1H), 3.53 (m, 4H), 3.33 (br. s, 3H), 2.67 (d, 4H, J=3 Hz), 2.33 (d, 2H, J=6 Hz), 2.04 (br. m, 3H), 1.93 (br. m, 6H), 1.58 (br. m, 8H), 1.46 (s, 7H), 1.40 (s, 8H), 1.25 (br. m, 11H), 1.07 (s, 5H), 0.99 (m, 5H), 0.87 (q, 10H), 0.75 (s, 3H).

### CN. Compound SA171: N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

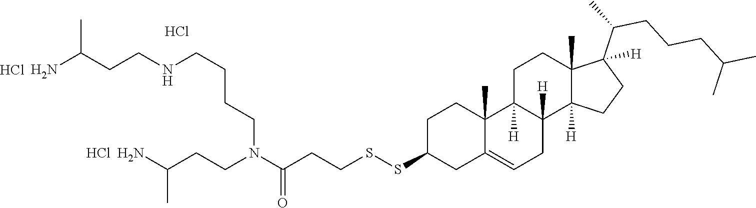

### Step 1: tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate

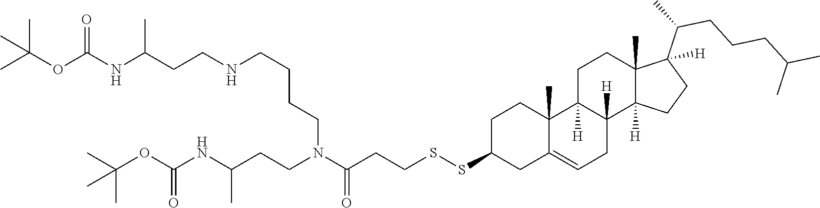

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.15 g, 0.30 mmol) in dry DCM (10 mL) stirring under nitrogen was added tert-butyl N-(4-{[4-({3-[(tert-butoxycarbonyl)amino]butyl}amino)butyl]amino}butan-2-yl)carbamate (0.32 g, 0.74 mmol), dimethylaminopyridine (0.07 g, 0.59 mmol), and 1-ethyl-3-(3-dimethylaminopropyl)carbodiimide hydrochloride (0.11 g, 0.59 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate as a light yellow oil (0.08 g, 0.09 mmol, 28.7%). UPLC/ELSD: RT: 2.79 min. MS (ES): m/z (MH⁺) 920.4 for C₅₂H₉₄N₄O₅S₂. ¹H NMR (300 MHz, CDCl₃) δ 5.37 (br. s, 1H), 4.79 (br. m, 2H), 3.64 (br. m, 3H), 3.31 (br. m, 4H), 2.95 (t, 2H), 2.69 (br. m, 7H), 2.34 (d, 2H, J=6 Hz), 2.24 (m, 1H), 1.94 (br. m, 4H), 1.60 (br. m, 11H), 1.44 (s, 22H), 1.31 (br. m, 5H), 1.16 (br. m, 13H), 1.00 (s, 6H), 0.93 (d, 4H, J=6 Hz), 0.88 (d, 4H, J=6 Hz), 0.68 (s, 3H).

### Step 2: N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

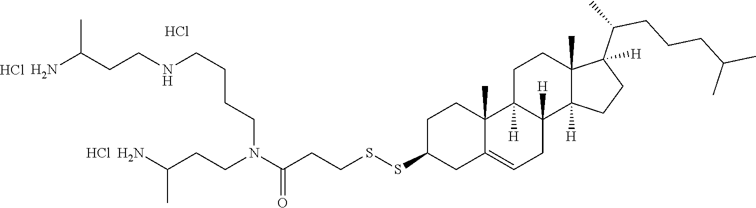

To a solution of tert-butyl (9-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-2,2,6-trimethyl-4-oxo-3-oxa-5,9,14-triazaoctadecan-17-yl)carbamate (0.08 g, 0.09 mmol) in DCM (2 mL) set stirring under nitrogen was added hydrochloric acid (4 M in dioxanes, 0.21 mL, 0.85 mmol) dropwise. The solution was allowed to stir at room temperature overnight. The following morning, hexanes (10 mL) was added to the mixture, which was cooled to 0° C. and allowed to stir for 30 minutes. The solution was then centrifuged for 20 minutes, the supernatant was discarded, and the white pellet was dried in vacuo to give N-(3-aminobutyl)-N-(4-((3-aminobutyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride as a white solid (0.05 g, 0.05 mmol, 63.1%). UPLC/ELSD: RT=1.77 min. MS (ES): m/z (MH⁺) 720.3 for C₄₂H₈₁Cl₃N₄₀S₂. ¹H NMR (300 MHz, MeOD) δ 5.39 (br. s, 1H), 3.68 (br. m, 1H), 3.46 (br. m, 6H), 3.33 (s, 4H), 3.14 (br. m, 6H), 3.00 (br. m, 5H), 2.63 (br. m, 1H), 2.37 (d, 2H, J=6 Hz), 1.99 (br. m, 16H), 1.53 (br. m, 8H), 1.40 (m, 13H), 1.18 (br. m, 8H), 1.05 (s, 4H), 0.98 (d, 5H, J=6 Hz), 0.90 (d, 8H, J=6 Hz), 0.74 (s, 3H).

### CO. Compound SA172: N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

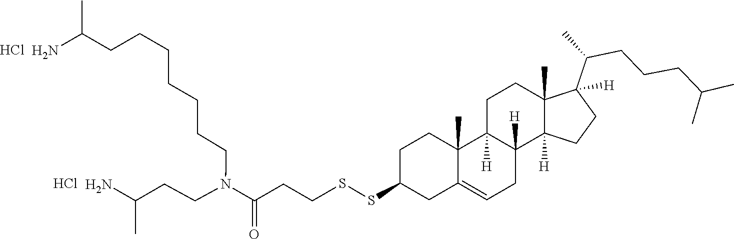

### Step 1: tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate

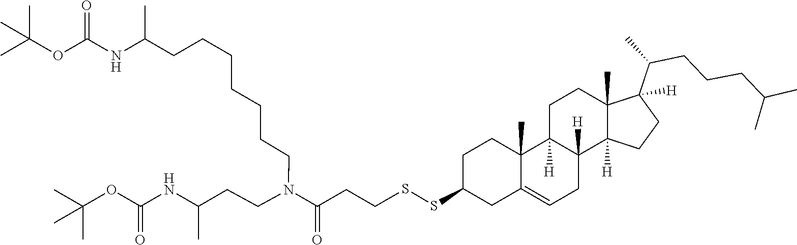

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.09 g, 0.18 mmol) in dry DCM (5 mL) stirring under nitrogen was added tert-butyl (9-((3-((tert-butoxycarbonyl)amino)butyl)amino)nonan-2-yl)carbamate (0.08 g, 0.18 mmol), dimethylaminopyridine (0.04 g, 0.35 mmol), and 1-ethyl-3-(3-dimethylaminopropyl)carbodiimide hydrochloride (0.07 g, 0.35 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate as a light yellow oil (0.11 g, 0.12 mmol, 66.1%). UPLC/ELSD: RT: 3.50 min. MS (ES): m/z (MH⁺) 919.4 for C₅₃H₉₅N₃O₅S₂. ¹H NMR (300 MHz, CDCl₃) δ 5.29 (br. s, 1H), 4.59 (br. s, 1H), 4.35 (br. s, 1H), 3.55 (br. m, 3H), 3.17 (br. m, 3H), 2.89 (t, 2H), 2.63 (br. m, 3H), 2.27 (br. m, 2H), 1.86 (m, 5H), 1.51 (br. m, 9H), 1.36 (s, 19H), 1.24 (br. m, 14H), 1.05 (m, 13H), 0.93 (s, 6H), 0.86 (d, 4H, J=6 Hz), 0.80 (d, 6H, J=6 Hz), 0.61 (s, 3H).

### Step 2: N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

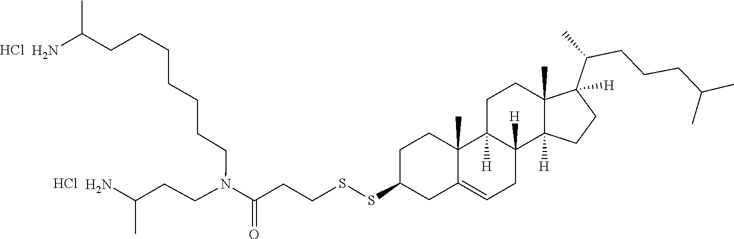

To a solution of tert-butyl (4-(N-(8-((tert-butoxycarbonyl)amino)nonyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamido)butan-2-yl)carbamate (0.11 g, 0.12 mmol) in DCM (3 mL) set stirring under nitrogen was added hydrochloric acid (4 N in dioxanes, 0.29 mL, 1.15 mmol) dropwise. The solution was allowed to stir at room temperature overnight. The following morning, hexanes (25 mL) was added to the mixture, which was cooled to 0° C. and allowed to stir for 30 minutes. The solution was then centrifuged for 20 minutes, the supernatant was discarded, and the white pellet was dried in vacuo to give N-(3-aminobutyl)-N-(8-aminononyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.09 g, 0.11 mmol, 94.3%). UPLC/ELSD: RT=1.86 min. MS (ES): m/z (MH⁺) 719.3 for C₄₃H₈₁Cl₂N₃OS₂. ¹H NMR (300 MHz, CDCl₃) δ 8.39 (br. m, 6H), 5.38 (br. s, 1H), 3.41 (br. m, 5H), 3.00 (br. s, 2H), 2.85 (br. s, 2H), 2.70 (br. m, 1H), 2.35 (br. m, 2H), 2.01 (m, 8H), 1.44 (br. m, 28H), 1.12 (br. m, 7H), 1.02 (s, 6H), 0.94 (d, 3H, J=6 Hz), 0.87 (d, 7H, J=6 Hz), 0.69 (s, 3H).

### CP. Compound SA173: N,N-bis(3-aminobutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

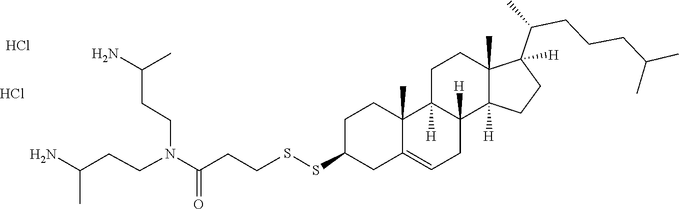

### Step 1: di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate

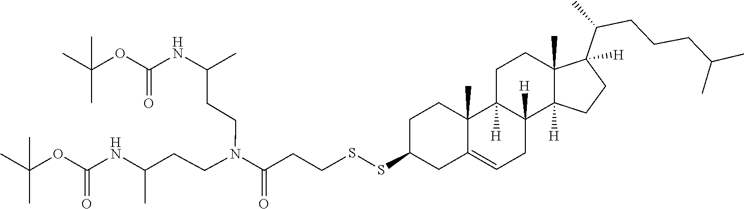

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.100 g, 0.197 mmol), tert-butyl N-[4-({3-[(tert-butoxycarbonyl)amino]butyl}amino)butan-2-yl]carbamate (0.078 g, 0.22 mmol), and triethylamine (0.08 mL, 0.6 mmol) in DCM (1.6 mL) cooled to 0° C. was added 50 wt % propanephosphonic acid anhydride in DCM (0.20 mL, 0.39 mmol) dropwise. The reaction mixture was stirred at room temperature and was monitored by LCMS. At 17 hours, the reaction mixture was diluted with DCM (10 mL), then washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (0-50% EtOAc in hexanes) to afford di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate (0.124 g, 0.146 mmol, 74.1%) as a clear oil. UPLC/ELSD: RT=3.60 min. MS (ES): m/z=849.65 (M+H)⁺ for C₄₈H₈₅N₃O₅S₂. ¹H NMR (300 MHz, CDCl₃) δ 5.39-5.32 (m, 1H), 4.68-4.38 (m, 2H), 3.77-3.12 (m, 6H), 3.06-2.85 (m, 2H), 2.80-2.54 (m, 3H), 2.41-2.23 (m, 2H), 2.03-0.94 (m, 54H), 1.00 (s, 3H), 0.91 (d, J=6.4 Hz, 3H), 0.87 (d, J=6.6 Hz, 3H), 0.86 (d, J=6.6 Hz, 3H), 0.67 (s, 3H).

### Step 2: N,N-bis(3-aminobutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

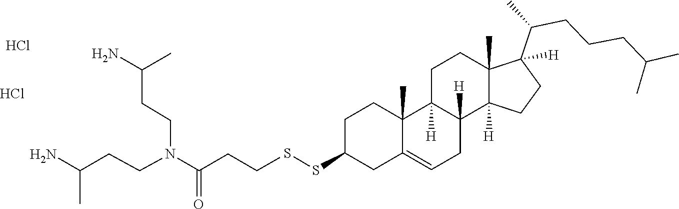

To a stirred solution of di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(butane-4,2-diyl))dicarbamate (0.121 g, 0.143 mmol) in DCM (2.5 mL) cooled to 0° C. was added 4 N HCl in dioxane (0.36 mL) dropwise. The reaction mixture was stirred at room temperature and was monitored by LCMS. At 16 hours, MTBE (20 mL) was added, and the reaction mixture was centrifuged (10,000×g for 15 min at 4° C.). The supernatant was removed, and the solids rinsed sparingly with MTBE. Solids were suspended in MTBE, then concentrated to afford N,N-bis(3-aminobutyl)-3-(((3 S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride (0.089 g, 0.12 mmol, 83.6%) as a white solid. UPLC/ELSD: RT=2.12 min. MS (ES): m/z=648.64 (M+H)⁺ for C₃₈H₆₉N₃OS₂. ¹H NMR (300 MHz, MeOD) δ 5.45-5.31 (m, 1H), 3.77-3.33 (m, 5H), 3.27-3.17 (m, 1H), 3.05-2.74 (m, 4H), 2.73-2.59 (m, 1H), 2.42-2.27 (m, 2H), 2.15-1.75 (m, 9H), 1.72-0.96 (m, 21H), 1.39 (d, J=6.8 Hz, 3H), 1.33 (d, J=6.5 Hz, 3H), 1.03 (s, 3H), 0.95 (d, J=6.4 Hz, 3H), 0.88 (d, J=6.6 Hz, 6H), 0.73 (s, 3H).

### CQ. Compound SA174: N,N-bis(3-amino-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

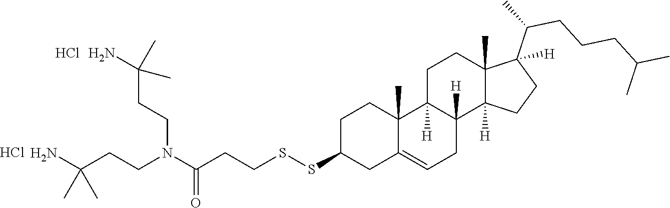

### Step 1: di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate

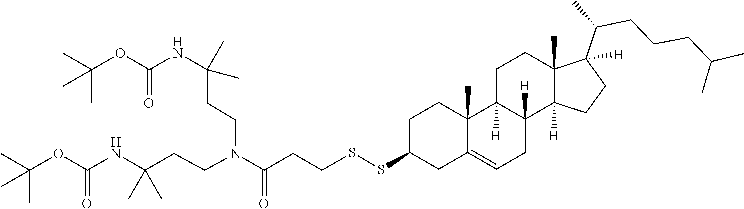

To a solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.13 g, 0.26 mmol) in dry DCM (5 mL) stirring under nitrogen was added di-tert-butyl (azanediylbis(2-methylbutane-4,2-diyl))dicarbamate (0.10 g, 0.26 mmol), dimethylaminopyridine (0.06 g, 0.52 mmol), and 1-ethyl-3-(3-dimethylaminopropyl)carbodiimide hydrochloride (0.10 g, 0.52 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate as a light yellow oil (0.21 g, 0.24 mmol, 92.9%). UPLC/ELSD: RT: 3.72 min. MS (ES): m/z (MH⁺) 877.4 for C₅₀H₈₉N₃O₅S₂. ¹H NMR (300 MHz, CDCl₃) δ 5.07 (br. s, 1H), 5.02 (s, 1H), 4.50 (s, 1H), 4.18 (s, 1H), 3.01 (br. m, 4H), 2.64 (t, 2H), 2.43 (br. m, 3H), 2.03 (br. m, 2H), 1.61 (br. m, 9H), 1.27 (br. m, 5H), 1.14 (s, 19H), 0.99 (s, 15H), 0.83 (br. m, 8H), 0.71 (s, 5H), 0.64 (d, 4H, J=6 Hz), 0.58 (d, 6H, J=6 Hz), 0.39 (s, 3H).

### Step 2: N,N-bis(3-amino-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride

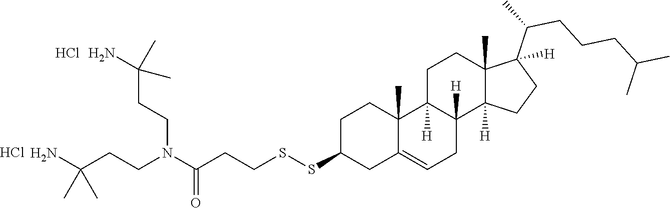

To a solution of di-tert-butyl (((3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.21 g, 0.24 mmol) in DCM (5 mL) set stirring under nitrogen was added hydrochloric acid (4 M in dioxanes, 0.60 mL, 2.40 mmol) dropwise. The solution was allowed to stir at room temperature overnight. The following morning, hexanes (15 mL) was added to the mixture, which was cooled to 0° C. and allowed to stir for 30 minutes. The solution was then centrifuged for 20 minutes, the supernatant was discarded, and the white pellet was dried in vacuo to give N,N-bis(3-amino-3-methylbutyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide dihydrochloride as a white solid (0.14 g, 0.17 mmol, 71.9%). UPLC/ELSD: RT=2.17 min. MS (ES): m/z (MH⁺) 677.3 for C₄₀H₇₅Cl₂N₃OS₂. ¹H NMR (300 MHz, MeOD) δ 5.40 (br. s, 1H), 3.51 (br. m, 4H), 3.32 (br. s, 2H), 2.99 (t, 2H), 2.86 (t, 2H), 2.64 (br. m, 1H), 2.37 (d, 2H, J=6 Hz), 2.07 (br. m, 9H), 1.55 (br. m, 8H), 1.47 (s, 6H), 1.41 (s, 8H), 1.18 (br. m, 11H), 1.05 (s, 5H), 0.96 (d, 4H, J=6 Hz), 0.92 (d, 7H, J=6 Hz), 0.74 (s, 3H).

### CR. Compound SA175: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

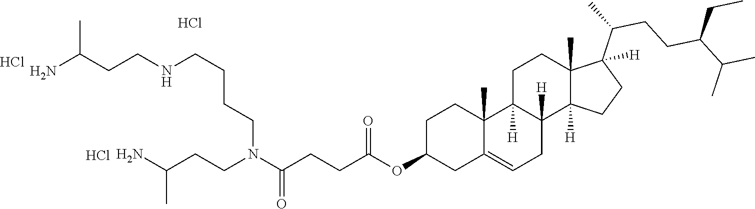

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate

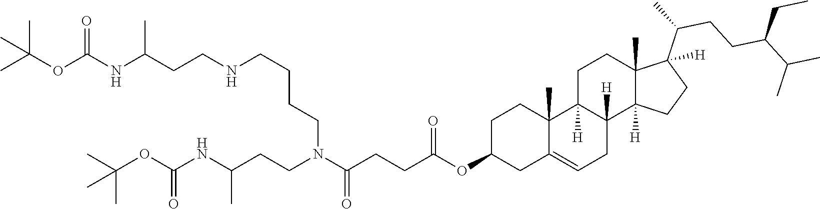

To a solution of 4-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-4-oxobutanoic acid (0.10 g, 0.19 mmol) in dry DCM (5 mL) stirring under nitrogen was added tert-butyl N-(4-{[4-({3-[(tert-butoxycarbonyl)amino]butyl}amino)butyl]amino}butan-2-yl)carbamate (0.21 g, 0.48 mmol), dimethylaminopyridine (0.05 g, 0.39 mmol), and 1-ethyl-3-(3-dimethylaminopropyl)carbodiimide hydrochloride (0.07 g, 0.39 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate as a light yellow oil (0.03 g, 0.03 mmol, 14.6%). UPLC/ELSD: RT: 2.76 min. MS (ES): m/z (MH⁺) 928.4 for C₅₅H₉₈N₄O₇. 1H NMR (300 MHz, CDCl₃) δ 5.30 (br. s, 1H), 4.70 (br. m, 1H), 4.53 (br. m, 2H), 3.57 (br. m, 2H), 3.25 (br. m, 4H), 2.57 (br. m, 8H), 2.26 (d, 3H, J=6 Hz), 1.77 (br. m, 6H), 1.54 (br. m, 13H), 1.37 (s, 20H), 1.17 (br. m, 5H), 1.08 (br. m, 12H), 0.94 (s, 5H), 0.86 (d, 5H, J=6 Hz), 0.78 (q, 9H), 0.61 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride

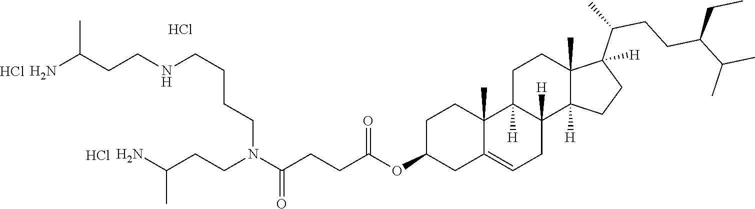

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)butyl)-2,2,6-trimethyl-4,15-dioxo-3-oxa-5,9,14-triazaoctadecan-18-oate (0.03 g, 0.03 mmol) in DCM (1 mL) set stirring under nitrogen was added hydrochloric acid (4 M in dioxanes, 0.07 mL, 0.28 mmol) dropwise. The solution was allowed to stir at room temperature overnight. The following morning, hexanes (5 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 4-((3-aminobutyl)(4-((3-aminobutyl)amino)butyl)amino)-4-oxobutanoate trihydrochloride as a white solid (0.02 g, 0.02 mmol, 63.9%). UPLC/ELSD: RT=1.75 min. MS (ES): m/z (MH⁺) 728.3 for C₄₅H₈₅C₃N₄O₃. ¹H NMR (300 MHz, MeOD) δ 5.40 (br. s, 1H), 4.55 (br. m, 1H), 3.68 (br. s, 1H), 3.50 (br. m, 4H), 3.33 (br. m, 3H), 3.14 (br. m, 5H), 2.66 (br. m, 4H), 2.33 (br. m, 3H), 1.81 (br. m, 19H), 1.37 (m, 11H), 1.20 (br. m, 6H), 1.07 (s, 5H), 0.99 (d, 5H, J=6 Hz), 0.89 (q, 9H), 0.75 (s, 3H).

### CS. Compound SA176: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

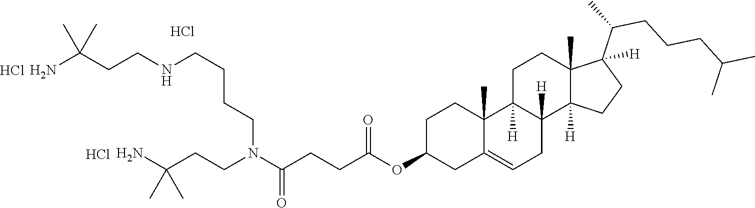

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate

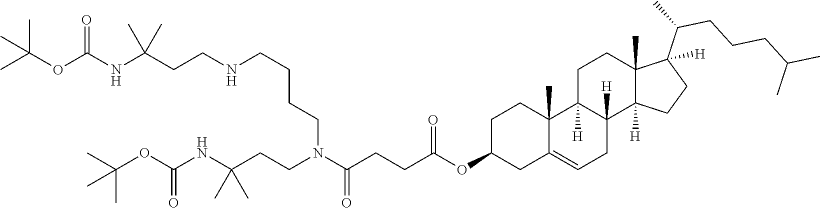

To a solution of 4-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-4-oxobutanoic acid (0.15 g, 0.31 mmol) in dry DCM (10 mL) stirring under nitrogen was added tert-butyl N-(4-{[4-({3-[(tert-butoxycarbonyl)amino]-3-methylbutyl}amino)butyl]amino}-2-methylbutan-2-yl)carbamate (0.35 g, 0.76 mmol), dimethylaminopyridine (0.8 g, 0.61 mmol), and 1-ethyl-3-(3-dimethylaminopropyl)carbodiimide hydrochloride (0.12 g, 0.61 mmol). The resulting solution was stirred at room temperature overnight. Then, the solution was diluted with dichloromethane, washed with saturated aqueous sodium bicarbonate (1×20 mL) and brine (1×20 mL), dried over sodium sulfate, filtered, and concentrated to an oil. The oil was taken up in DCM and purified on silica in DCM with a 0-100% (80:19:1 DCM/MeOH/NH₄OH) gradient. Product-containing fractions were pooled and concentrated to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate as a light yellow oil (0.10 g, 0.10 mmol, 33.6%). UPLC/ELSD: RT: 2.70 min. MS (ES): m/z (MH⁺) 928.4 for C₅₅H₉₈N₄O₇. ¹H NMR (301 MHz, CDCl₃) δ 5.70 (br. s, 1H), 5.29 (br. s, 1H), 4.50 (br. m, 2H), 3.21 (br. m, 4H), 2.54 (br. m 8H), 2.24 (d, 2H, J=6 Hz), 1.87 (br. m, 7H), 1.53 (br. m, 10H), 1.36 (s, 22H), 1.23 (s, 15H), 1.02 (br. m, 7H), 0.94 (s, 6H), 0.85 (d, 4H, J=6 Hz), 0.80 (d, 6H, J=6 Hz), 0.60 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride

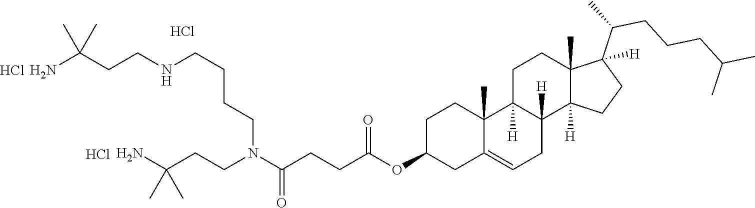

To a solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-methylbutyl)-2,2,6,6-tetramethyl-4,15-dioxo-3-oxa-5,9,14-triazaoctadecan-18-oate (0.10 g, 0.10 mmol) in DCM (2 mL) set stirring under nitrogen was added hydrochloric acid (4 M in dioxanes, 0.26 mL, 1.02 mmol) dropwise. The solution was allowed to stir at room temperature overnight. The following morning, hexanes (5 mL) was added to the mixture, which was cooled to 0° C. and allowed to stir for 30 minutes. The solution was then centrifuged for 20 minutes, the supernatant was discarded, and the white pellet was dried in vacuo to give (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 4-((3-amino-3-methylbutyl)(4-((3-amino-3-methylbutyl)amino)butyl)amino)-4-oxobutanoate trihydrochloride as a white solid (0.09 g, 0.08 mmol, 74.4%). UPLC/ELSD: RT=1.72 min. MS (ES): m/z (MH⁺) 728.3 for C₄₅H₈₅C₃N₄O₃. ¹H NMR (300 MHz, MeOD) δ 5.40 (br. s, 1H), 4.54 (br. m, 1H), 3.69 (br. s, 1H), 3.49 (br. m, 4H), 3.32 (br. s, 6H), 3.17 (br. m, 5H), 2.66 (br. m, 4H), 2.33 (br. m, 2H), 2.05 (br. m, 10H), 1.66 (br. m, 16H), 1.43 (br. m, 16H), 1.32 (br. s, 16H), 1.16 (br. m, 8H), 1.07 (s, 5H), 0.92 (br. m, 25H), 0.75 (s, 3H).

### CT. Compound SA177: N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

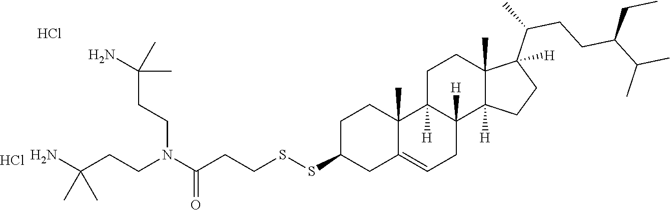

### Step 1: Sitosteryl Chloride

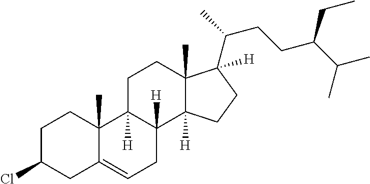

Sitosterol (3.000 g, 7.234 mmol) and thionyl chloride (3.00 mL, 41.4 mmol) were combined in PhMe (30 mL). The reaction mixture was stirred at 80° C. and was monitored by TLC. At 20 hours, the reaction mixture was concentrated, then re-concentrated from PhMe (2×). The solids were dissolved in hot 3:1 EtOH/EtOAc (45 mL), then allowed to cool to room temperature. Solid precipitated out of solution. The mother liquor was decanted, and the solids were rinsed sparingly with cold 3:1 EtOH/EtOAc to afford sitosteryl chloride (2.534 g, 5.850 mmol, 80.9%) as a clear solid. UPLC/ELSD: RT=3.74 min. ¹H NMR (300 MHz, CDCl₃) δ 5.41-5.33 (m, 1H), 3.87-3.67 (m, 1H), 2.69-2.39 (m, 2H), 2.17-1.75 (m, 6H), 1.73-0.76 (m, 30H), 1.03 (s, 3H), 0.92 (d, J=6.5 Hz, 3H), 0.68 (s, 3H). ¹³C NMR (75 MHz, CDCl₃) δ 140.96, 122.64, 60.50, 56.85, 56.19, 50.22, 45.98, 43.56, 42.46, 39.85, 39.27, 36.53, 36.29, 34.09, 33.53, 31.98, 31.93, 29.30, 28.38, 26.22, 24.43, 23.22, 21.11, 19.98, 19.41, 19.19, 18.93, 12.13, 12.00.

### Step 2: Sitosteryl Thiocyanate

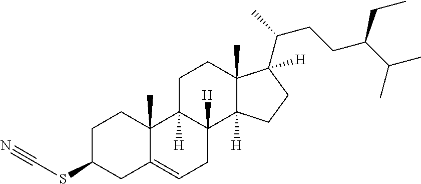

Sitosteryl chloride (2.748 g, 6.344 mmol) and sodium thiocyanate (19.235 g, 237.27 mmol) were refluxed in EtOH (105 mL). The reaction was monitored by TLC. At 64 hours, the reaction mixture was filtered hot, rinsing with a copious amount of DCM. The filtrate was concentrated, taken up in DCM (150 mL), and washed with water. The organic layer was passed through a hydrophobic frit, dried over Na₂SO₄, and concentrated. The resultant solid was dissolved in near boiling 1:1 EtOAc/hexanes (19 mL), then allowed to cool slowly. Once reaching room temperature, the mixture was further cooled to 4° C. Solids were collected by vacuum filtration, rinsing sparingly with cold 1:1 EtOAc/hexanes to afford sitosteryl thiocyanate (2.183 g, 4.789 mmol, 75.5%) as an off-white solid. UPLC/ELSD: RT=3.47 min. ¹H NMR (300 MHz, CDCl₃) δ 5.48-5.35 (m, 1H), 3.23-2.97 (m, 1H), 2.65-2.35 (m, 2H), 2.10-0.76 (m, 36H), 1.03 (s, 3H), 0.92 (d, J=6.4 Hz, 3H), 0.68 (s, 3H). ¹³C NMR (75 MHz, CDCl₃) δ 140.10, 123.29, 111.40, 56.80, 56.18, 50.20, 48.23, 45.98, 42.45, 39.89, 39.79, 39.51, 36.61, 36.28, 34.08, 31.96, 31.86, 30.10, 29.29, 28.37, 26.21, 24.41, 23.21, 21.06, 19.97, 19.35, 19.18, 18.93, 12.13, 12.00.

### Step 3: Thiositosterol

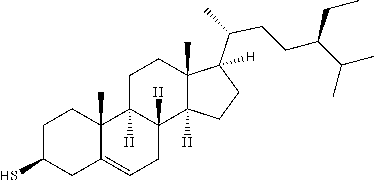

To a stirred solution of THF (30 mL) and 2.3 M lithium aluminum hydride in 2-methyltetrahydrofuran (4.7 mL) was added dropwise a solution of sitosteryl thiocyanate (2.100 g, 4.607 mmol) in PhMe (20 mL) dropwise over 15 min. The reaction mixture was stirred at room temperature and was monitored by TLC. At 2.5 hours, the reaction mixture was cooled to 0° C., then aq. 3 N HCl (50 mL) was added slowly dropwise over 10 min. Upon completion of addition, the layers were separated. The aqueous layer was extracted with MTBE (3×30 mL). The combined organics layers were washed with water and brine, dried over Na₂SO₄, and concentrated to afford thiositosterol (1.944 g, 4.513 mmol, 97.9%) as a white solid. UPLC/ELSD: RT=3.70 min. ¹H NMR (300 MHz, CDCl₃) δ 5.43-5.22 (m, 1H), 2.80-2.60 (m, 1H), 2.45-2.23 (m, 2H), 2.10-1.75 (m, 5H), 1.74-0.78 (m, 32H), 1.00 (s, 3H), 0.92 (d, J=6.4 Hz, 3H), 0.67 (s, 3H). ¹³C NMR (75 MHz, CDCl₃) δ 142.07, 121.19, 56.90, 56.20, 50.35, 45.98, 44.35, 42.45, 40.08, 39.89, 39.60, 36.49, 36.30, 34.22, 34.09, 31.95, 29.29, 28.39, 26.21, 24.43, 23.21, 21.04, 19.98, 19.48, 19.18, 18.93, 12.13, 12.00.

### Step 4: 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine

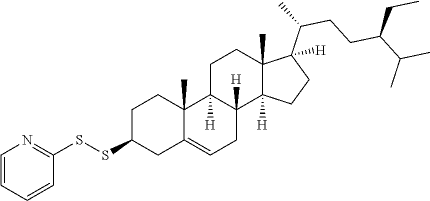

Thiositosterol (1.92 g, 4.46 mmol) and Aldrithiol (1.08 g, 4.90 mmol) were combined in chloroform (12 mL). The reaction mixture was stirred at room temperature and was monitored by LCMS. At 24 hours, Aldrithiol (0.25 g) was added. At 6 days, the reaction mixture was concentrated, taken up in MeOH (30 mL), and sonicated. The solids were collected by vacuum filtration rinsing sparingly with MeOH to afford 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine (2.098 g, 3.886 mmol, 87.2%) as a light yellow solid. UPLC/ELSD: RT=3.56 min. MS (ES): m/z=540.62 (M+H)⁺ for C₃₄H₅₃NS₂. ¹H NMR (300 MHz, CDCl₃) δ 8.51-8.39 (m, 1H), 7.83-7.71 (m, 1H), 7.69-7.57 (m, 1H), 7.13-7.00 (m, 1H), 5.40-5.28 (m, 1H), 2.89-2.69 (m, 1H), 2.41-2.28 (m, 2H), 2.08-1.75 (m, 5H), 1.74-0.74 (m, 31H), 0.98 (s, 3H), 0.91 (d, J=6.3 Hz, 3H), 0.67 (s, 3H).

### Step 5: 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate

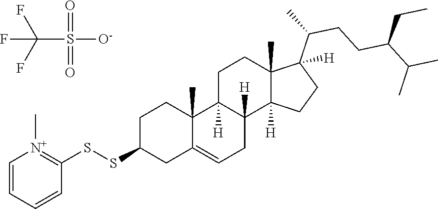

To a solution of 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)pyridine (2.000 g, 3.704 mmol) in heptanes (30 mL) and DCM (3.0 mL) was added methyl trifluoromethanesulfonate (0.51 mL, 4.5 mmol) dropwise over 10 min. The reaction mixture was stirred at room temperature and was monitored by TLC. At 19 hours, solids were collected by vacuum filtration rinsing with heptanes to afford 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.443 g, 3.470 mmol, 93.7%) as a white solid. UPLC/ELSD: RT=2.67 min. MS (ES): m/z=554.80 (M)⁺ for C₃₅H₅₆NS₂. ¹H NMR (300 MHz, CD₃CN) δ 8.60-8.49 (m, 2H), 8.39-8.30 (m, 1H), 7.75-7.66 (m, 1H), 5.40-5.35 (m, 1H), 4.19 (s, 3H), 3.04-2.89 (m, 1H), 2.49-2.32 (m, 2H), 2.08-0.76 (m, 36H), 1.01 (s, 3H), 0.93 (d, J=6.5 Hz, 3H), 0.69 (s, 3H).

### Step 6: 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid

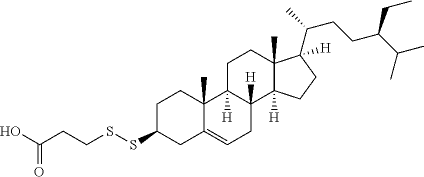

To a solution of 2-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-1-methylpyridin-1-ium trifluoromethanesulfonate (2.400 g, 3.409 mmol) in DMF (15 mL) was added 3-mercaptopropionic acid (0.34 mL, 3.9 mmol). The reaction mixture was stirred at room temperature and was monitored by LCMS. At 23 hours, the reaction mixture was poured into water (30 mL) and sonicated. The solids were collected by vacuum filtration rinsing with water. The solids were dissolved in DCM, passed through a hydrophobic frit, dried over Na₂SO₄, and concentrated. ACN (15 mL) was added to the residue. The suspension was sonicated and cooled in an ice bath. Then the solids were collected by vacuum filtration rinsing sparingly with cold ACN. The solids were taken up in ACN (15 mL) and sonicated. Solids were collected by vacuum filtration rinsing with ACN to afford 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (1.333 g, 2.492 mmol, 73.1%) as a white solid. UPLC/ELSD: RT=3.28 min. ¹H NMR (300 MHz, CDCl₃) δ 11.10 (br. s, 1H), 5.46-5.25 (m, 1H), 2.96-2.85 (m, 2H), 2.84-2.74 (m, 2H), 2.74-2.56 (m, 1H), 2.42-2.22 (m, 2H), 2.09-1.75 (m, 5H), 1.75-0.75 (m, 31H), 1.00 (s, 3H), 0.92 (d, J=6.5 Hz, 3H), 0.68 (s, 3H).

### Step 7: di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate

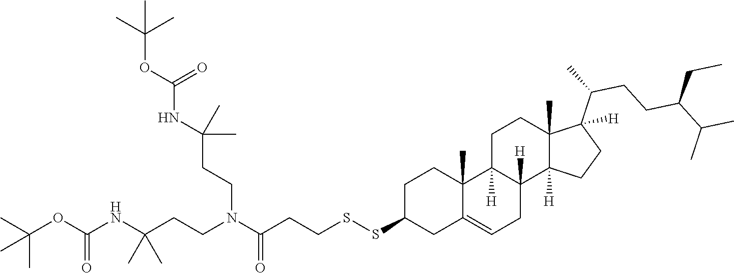

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.110 g, 0.206 mmol), tert-butyl N-[4-({3-[(tert-butoxycarbonyl)amino]-3-methylbutyl}amino)-2-methylbutan-2-yl]carbamate (0.088 g, 0.226 mmol), and triethylamine (0.09 mL, 0.6 mmol) in DCM (1.1 mL) cooled to 0° C. was added 50 wt % propanephosphonic acid anhydride in DCM (0.21 mL, 0.41 mmol) dropwise. The reaction mixture was stirred at room temperature and was monitored by LCMS. At 19 hours, the reaction mixture was diluted with DCM to 10 mL, then washed with 5% aq. NaHCO₃ soln. The aqueous was extracted with DCM (10 mL). The combined organics were washed with water, passed through a hydrophobic frit, dried over Na₂SO₄, and concentrated. The crude material was purified via silica gel chromatography (20-50% EtOAc in hexanes) to afford di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.139 g, 0.154 mmol, 74.7%) as a clear oil. UPLC/ELSD: RT=3.65 min. MS (ES): m/z=905.77 (M+H)⁺ for C₅₂H₉₃N₃O₅S₂. ¹H NMR (300 MHz, CDCl₃) δ 5.39-5.30 (m, 1H), 4.73 (s, 1H), 4.39 (s, 1H), 3.40-3.18 (m, 4H), 3.04-2.90 (m, 2H), 2.78-2.56 (m, 3H), 2.42-2.22 (m, 2H), 2.11-0.78 (m, 70H), 1.00 (s, 3H), 0.92 (d, J=6.3 Hz, 3H), 0.68 (s, 3H).

### Step 8: N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

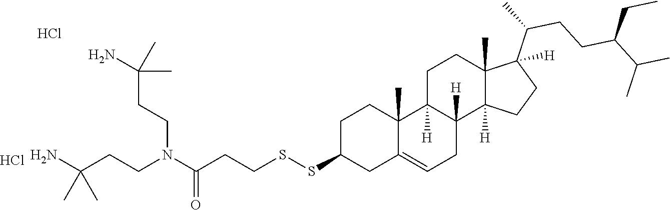

To a solution of di-tert-butyl (((3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)azanediyl)bis(2-methylbutane-4,2-diyl))dicarbamate (0.131 g, 0.145 mmol) in DCM (2.0 mL) was added 4 N HCl in dioxane (0.26 mL) dropwise. The reaction mixture was stirred at room temperature and was monitored by LCMS. At 19 hours, the reaction mixture was diluted with MBTE to 30 mL, then was centrifuged (10,000×g for 15 min at 4° C.). The supernatant was drawn off. The solids were suspended in MTBE, then concentrated to afford N,N-bis(3-amino-3-methylbutyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.089 g, 0.109 mmol, 75.4%) as a white solid. UPLC/ELSD: RT=2.25 min. MS (ES): m/z=373.20 [(M+2H)+CH₃CN]²⁺ for C₄₂H₇₇N₃OS₂. ¹H NMR (300 MHz, MeOD) δ 5.46-5.31 (m, 1H), 3.61-3.39 (m, 4H), 3.01-2.91 (m, 2H), 2.88-2.77 (m, 2H), 2.74-2.58 (m, 1H), 2.40-2.24 (m, 2H), 2.16-1.79 (m, 9H), 1.77-0.78 (m, 31H), 1.44 (s, 6H), 1.39 (s, 6H), 1.03 (s, 3H), 0.96 (d, J=6.4 Hz, 3H), 0.73 (s, 3H).

### CU. Compound SA178: N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

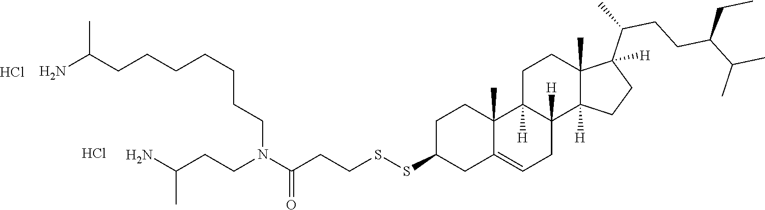

### Step 1: tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate

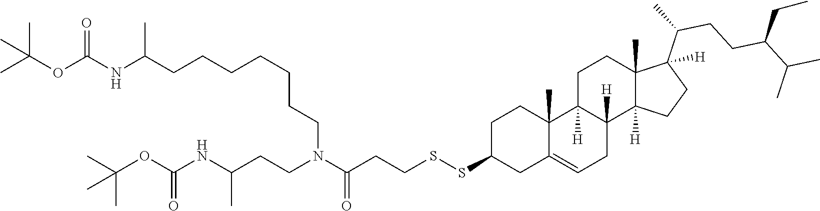

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.100 g, 0.187 mmol), tert-butyl N-[4-({8-[(tert-butoxycarbonyl)amino]nonyl}amino)butan-2-yl]carbamate (0.088 g, 0.206 mmol), and triethylamine (0.08 mL, 0.569 mmol) in DCM (2.5 mL) cooled to 0° C. was added 50 wt % propanephosphonic acid anhydride DCM (0.19 mL, 0.37 mmol) dropwise. The reaction mixture stirred at room temperature. At 19 hours, the reaction mixture was diluted with DCM to 10 mL, then washed with 5% aq. NaHCO₃soln. The aqueous was extracted with DCM (10 mL). The combined organic layers were passed through a hydrophobic frit, dried over Na₂SO₄, and concentrated. The crude material was purified via silica gel chromatography (20-50% EtOAc in hexanes) to afford tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate (0.130 g, 0.137 mmol, 73.5%) as a white foam. UPLC/ELSD: RT=3.65 min. MS (ES): m/z=946.96 (M+H)⁺ for C₅₅H₉₉N₃O₅S₂. ¹H NMR (300 MHz, CDCl₃) δ 5.45-5.29 (m, 1H), 4.70-4.18 (m, 2H), 3.80-3.10 (m, 6H), 3.05-2.87 (m, 2H), 2.83-2.56 (m, 3H), 2.43-2.25 (m, 2H), 2.14-0.75 (m, 74H), 1.00 (s, 3H), 0.92 (d, J=6.4 Hz, 3H), 0.67 (s, 3H).

### Step 2: N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

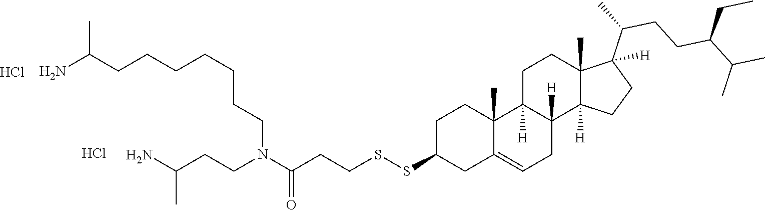

To a stirred solution of tert-butyl (4-(N-(8-((tert-butoxycarbonyl)amino)nonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamido)butan-2-yl)carbamate (0.126 g, 0.133 mmol) in DCM (1.9 mL) was added 4 N HCl in dioxane (0.24 mL) dropwise. The reaction mixture was stirred at room temperature and was monitored by LCMS. At 19 hours, the reaction mixture was diluted with MTBE (30 mL), and the reaction mixture was centrifuged (10,000×g for 15 min at 4° C.). The supernatant was drawn off, and the solids were suspended in MTBE and concentrated to afford N-(3-aminobutyl)-N-(8-aminononyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.101 g, 0.115 mmol, 86.7%). UPLC/ELSD: RT=2.28 min. MS (ES): m/z=373.82 (M+2H)²⁺ for C₄₅H₈₃N₃OS₂. ¹H NMR (300 MHz, MeOD) δ 5.42-5.33 (m, 1H), 3.76-3.59 (m, 1H), 3.56-3.10 (m, 5H), 3.03-2.57 (m, 5H), 2.42-2.25 (m, 2H), 2.12-0.78 (m, 56H), 1.04 (s, 3H), 0.96 (d, J=6.4 Hz, 3H), 0.73 (s, 3H).

### CV. Compound SA179: N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

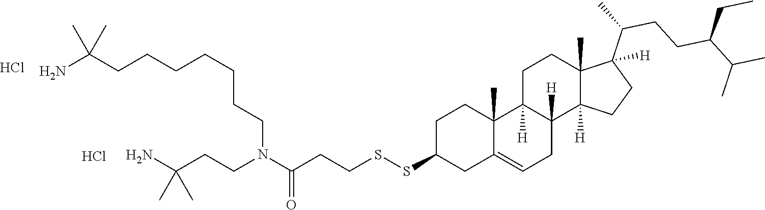

### Step 1: tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate

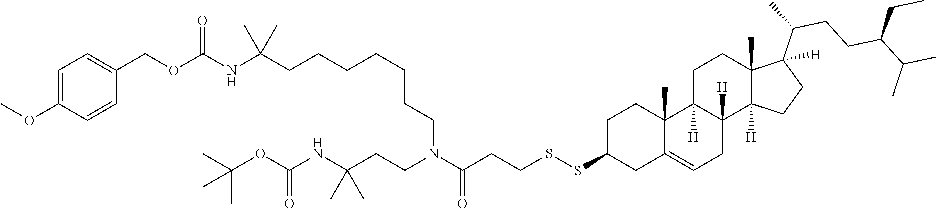

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.102 g, 0.191 mmol), tert-butyl N-(4-{[8-({[(4-methoxyphenyl)methoxy]carbonyl}amino)-8-methylnonyl]amino}-2-methylbutan-2-yl)carbamate (0.113 g, 0.217 mmol), and triethylamine (0.08 mL, 0.6 mmol) in DCM (2.5 mL) cooled to 0° C. was added 50% propanephosphonic acid anhydride in DCM (0.19 mL, 0.37 mmol) dropwise. The reaction mixture was stirred at room temperature and was monitored by LCMS. At 19 hours, the reaction mixture was diluted with DCM to 10 mL, then washed with 5% aq. NaHCO₃ soln. The aqueous was extracted with DCM (10 mL). The combined organics were passed through a hydrophobic frit, dried over Na₂SO₄, and concentrated. The crude material was purified via silica gel chromatography (20-50% EtOAc in hexanes) to afford tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate (0.160 g, 0.154 mmol, 80.8%) as a clear oil. UPLC/ELSD: RT=3.68 min. MS (ES): m/z=1039.59 (M+H)⁺ for C₆₁H₁₀₃N₃O₆S₂. ¹H NMR (300 MHz, CDCl₃) δ 7.33-7.27 (m, 2H), 6.92-6.85 (m, 2H), 5.39-5.30 (m, 1H), 4.97 (s, 2H), 4.71-4.35 (m, 2H), 3.81 (s, 3H), 3.38-3.16 (m, 4H), 3.01-2.89 (m, 2H), 2.79-2.55 (m, 3H), 2.39-2.25 (m, 2H), 2.09-0.77 (m, 71H), 1.00 (s, 3H), 0.92 (d, J=6.4 Hz, 3H), 0.68 (s, 3H).

### Step 2: N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride

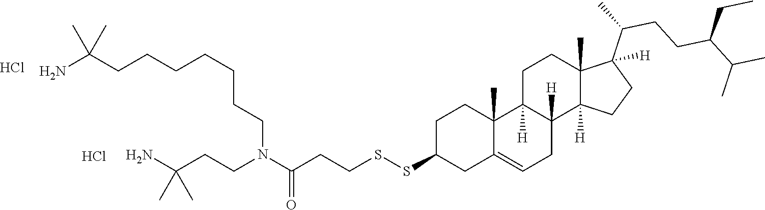

To a solution of tert-butyl (4-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)-N-(8-((((4-methoxybenzyl)oxy)carbonyl)amino)-8-methylnonyl)propanamido)-2-methylbutan-2-yl)carbamate (0.154 g, 0.148 mmol) in DCM (2.4 mL) was added 4 N HCl in dioxane (0.26 mL) dropwise. The reaction mixture was stirred at room temperature and was monitored by LCMS. At 19 hours, the reaction mixture was diluted with MTBE to 30 mL, then was centrifuged (10,000×g for 15 min at 4° C.). The supernatant was drawn off. The solids were suspended in MTBE (30 mL), then centrifuged (10,000×g for 15 min at 4° C.). The supernatant was drawn off. The solids were suspended in MBTE and then concentrated to afford N-(3-amino-3-methylbutyl)-N-(8-amino-8-methylnonyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide dihydrochloride (0.042 g, 0.047 mmol, 31.6%) as a white solid. UPLC/ELSD: RT=2.33 min. MS (ES): m/z=388.12 (M+2H)² for C₄₇H₇N₃OS₂. ¹H NMR (300 MHz, MeOD) δ 5.45-5.33 (m, 1H), 3.52-3.34 (m, 4H), 3.02-2.89 (m, 2H), 2.87-2.74 (m, 2H), 2.73-2.57 (m, 1H), 2.40-2.26 (m, 2H), 2.13-1.79 (m, 7H), 1.37 (s, 6H), 1.33 (s, 6H), 1.77-0.78 (m, 43H), 1.04 (s, 3H), 0.96 (d, J=6.4 Hz, 3H), 0.73 (s, 3H).

### CW. Compound SA180: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride

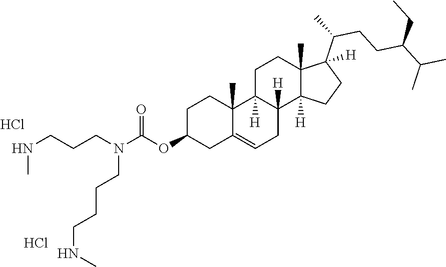

### Step 1: tert-butyl N-methyl-N-[4-(2-nitrobenzenesulfonamido)butyl]carbamate

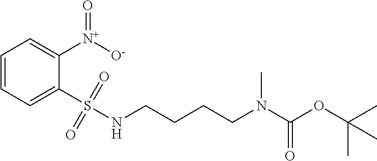 [SMILES: CN(CCCCNS(=O)(=O)c1ccccc1[N+](=O)[O-])C(=O)OC(C)(C)C]

To a stirred solution of tert-butyl N-(4-aminobutyl)-N-methylcarbamate (1.000 g, 4.943 mmol) and triethylamine (0.95 mL, 6.8 mmol) in DCM (15 mL) cooled to 0° C. was added dropwise a solution of 2-nitrobenzenesulfonyl chloride (1.315 g, 5.932 mmol) in DCM (5 mL). After addition, the reaction mixture was stirred at room temperature and was monitored by TLC. At 17 hours, the reaction mixture was cooled to 0° C., then 5% aq. NaHCO₃ soln. (10 mL) was added. After warming to room temperature, the layers were separated. The aqueous layer was extracted with DCM (15 mL). The combined organics were washed with 5% aq. citric acid soln. and water (2×), passed through a hydrophobic frit, dried over Na₂SO₄, and concentrated to afford tert-butyl N-methyl-N-[4-(2-nitrobenzenesulfonamido)butyl]carbamate (1.932 g, quant.) as an amber oil. UPLC/ELSD: RT=0.76 min. MS (ES): m/z=288.09 [(M+H)—(CH₃)₂C═CH₂—CO₂]⁺ for C₁₆H₂₅N₃O₆S. ¹H NMR (300 MHz, CDCl₃) δ 8.18-8.08 (m, 1H), 7.90-7.80 (m, 1H), 7.80-7.68 (m, 2H), 5.34 (br. s, 1H), 3.23-3.06 (m, 4H), 2.79 (s, 3H), 1.60-1.46 (m, 4H), 1.43 (s, 9H).

### Step 2: tert-butyl N-[3-(N-{4-[(tert-butoxycarbonyl)(methyl)amino]butyl}-2-nitrobenzenesulfonamido)propyl]-N-methylcarbamate

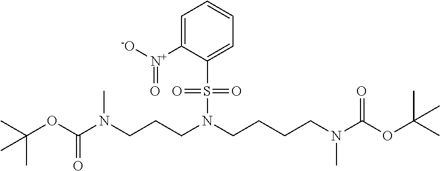

Tert-butyl N-methyl-N-[4-(2-nitrobenzenesulfonamido)butyl]carbamate (0.750 g, 1.94 mmol), tert-butyl N-(3-bromopropyl)-N-methylcarbamate (0.586 g, 2.32 mmol), and potassium carbonate (0.535 g, 3.87 mmol) were combined in DMF (11.25 mL). The reaction mixture was stirred at 80° C. and was monitored by LCMS. At 19 hours, the reaction mixture was filtered rinsing with EtOAc. The filtrate was diluted with EtOAc to 150 mL, then washed with 5% aq. NaHCO₃ soln., water (3×), and brine. The organics were dried over Na₂SO₄ and concentrated. The crude material was purified via silica gel chromatography (20-80% EtOAc in hexanes) to afford tert-butyl N-[3-(N-{4-[(tert-butoxycarbonyl)(methyl)amino]butyl}-2-nitrobenzenesulfonamido)propyl]-N-methylcarbamate (0.836 g, 1.50 mmol, 82.8%) as a white solid. UPLC/ELSD: RT=1.46 min. MS (ES): m/z=559.37 (M+H)⁺ for C₂₅H₄₂N₄O₈S. ¹H NMR (300 MHz, CDCl₃) δ 8.06-7.93 (m, 1H), 7.76-7.55 (m, 3H), 3.42-3.10 (m, 8H), 2.80 (s, 6H), 1.89-1.70 (m, 2H), 1.70-1.46 (m, 4H), 1.44 (s, 18H).

### Step 3: tert-butyl N-[3-({4-[(tert-butoxycarbonyl)(methyl)amino]butyl}amino)propyl]-N-methylcarbamate

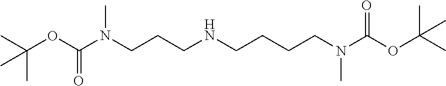 [SMILES: CN(CCCCNCCCN(C)C(=O)OC(C)(C)C)C(=O)OC(C)(C)C]

To a mixture of tert-butyl N-[3-(N-{4-[(tert-butoxycarbonyl)(methyl)amino]butyl}-2-nitrobenzenesulfonamido)propyl]-N-methylcarbamate (0.830 g, 1.49 mmol) and potassium carbonate (0.616 g, 4.46 mmol) in DMF (12.5 mL) was added thiophenol (0.28 mL, 2.7 mmol). The reaction mixture was stirred at room temperature and was monitored by LCMS. At 23 hours, the reaction mixture was filtered rinsing with EtOAc. The filtrate was diluted with EtOAc to 150 mL, then washed with 5% aq. K₂CO₃ (2×), water (3×), and brine. The organics were dried over Na₂SO₄, and then concentrated. The crude material was purified via silica gel chromatography (0-20% (5% conc. aq. NH₄OH in MeOH) in DCM) to afford tert-butyl N-[3-({4-[(tert-butoxycarbonyl)(methyl)amino]butyl}amino)propyl]-N-methylcarbamate (0.477 g, 1.28 mmol, 86.0%) as a yellow oil. UPLC/ELSD: RT=0.45 min. MS (ES): m/z=374.56 (M+H)⁺ for C₁₉H₃₉N₃O₄. ¹H NMR (300 MHz, CDCl₃) δ 3.33-3.11 (m, 4H), 2.83 (s, 6H), 2.70-2.50 (m, 4H), 1.76-1.62 (m, 2H), 1.62-1.37 (m, 22H).

### Step 4: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate

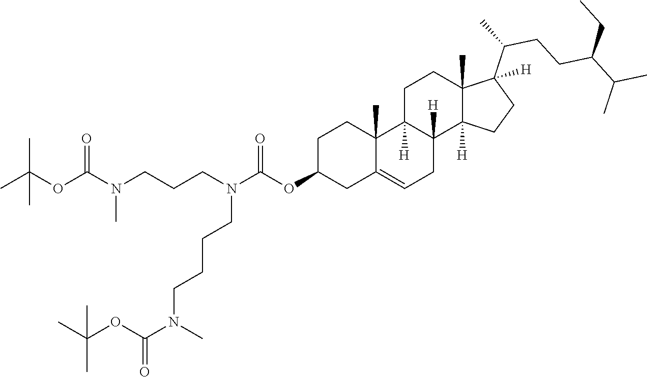

Sitosteryl 4-nitrophenyl carbonate (0.120 g, 0.207 mmol), tert-butyl N-[3-({4-[(tert-butoxycarbonyl)(methyl)amino]butyl}amino)propyl]-N-methylcarbamate (0.077 g, 0.207 mmol), and triethylamine (0.09 mL, 0.6 mmol) were combined in PhMe (1.8 mL). The reaction mixture was stirred at 90° C. and was monitored by LCMS. At 24 hours, the reaction mixture was cooled to room temperature, diluted with DCM to 10 mL, and washed with 5% aq. K₂CO₃ soln. (2×). The aqueous was extracted with DCM (10 mL). The combined organics were passed through a hydrophobic frit, dried over Na₂SO₄, and concentrated. The crude material was purified via silica gel chromatography (0-50% EtOAc in hexanes) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate (0.152 g, 0.187 mmol, 90.2%) as a clear oil. UPLC/ELSD: RT=3.59 min. MS (ES): m/z=814.88 (M+H)⁺ for C₄₉H₈₇N₃O₆. ¹H NMR (300 MHz, CDCl₃) δ 5.41-5.33 (m, 1H), 4.58-4.42 (m, 1H), 3.32-3.10 (m, 8H), 2.88-2.79 (m, 6H), 2.41-2.20 (m, 2H), 2.09-0.75 (m, 60H), 1.02 (s, 3H), 0.92 (d, J=6.4 Hz, 3H), 0.68 (s, 3H).

### Step 5: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride

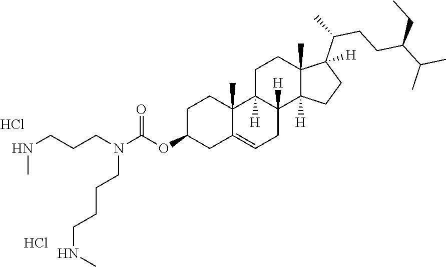

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-((tert-butoxycarbonyl)(methyl)amino)butyl)(3-((tert-butoxycarbonyl)(methyl)amino)propyl)carbamate (0.143 g, 0.176 mmol) in DCM (2.2 mL) was added 4 N HCl in dioxane (0.31 mL). The reaction mixture was stirred at room temperature and was monitored by LCMS. At 18 hours, the reaction mixture was diluted with MTBE to 20 mL, then centrifuged (10,000×g for 30 min at 4° C.). The supernatant was drawn off. The solids were rinsed with MTBE, suspended in MTBE, and concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (4-(methylamino)butyl)(3-(methylamino)propyl)carbamate dihydrochloride (0.102 g, 0.131 mmol, 74.8%) as a white solid. UPLC/ELSD: RT=2.03 min. MS (ES): m/z=328.45 [(M+2H)+CH₃CN]²⁺ for C₃₉H₇₁N₃O₂. ¹H NMR (300 MHz, MeOD) δ 5.44-5.33 (m, 1H), 4.52-4.37 (m, 1H), 3.47-3.33 (m, 4H), 3.10-2.90 (m, 4H), 2.78-2.63 (m, 6H), 2.44-2.27 (m, 2H), 2.19-0.78 (m, 42H), 1.06 (s, 3H), 0.96 (d, J=6.4 Hz, 3H), 0.73 (s, 3H).

### CX. Compound SA181: N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride

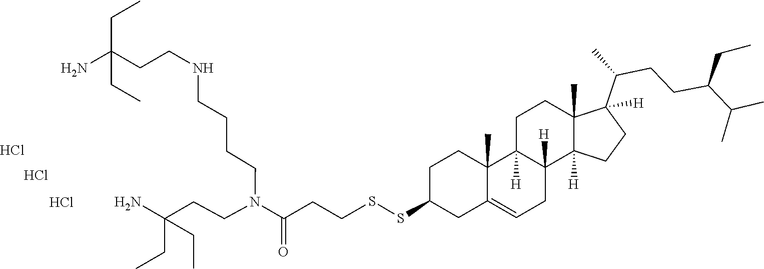

### Step 1: tert-Butyl (6,6,17-triethyl-9-(3-(((3S,8S,9S,10R,13R,14S,17R)-17-((2R,5R)-5-ethyl-6-methylheptan-2-yl)-10,13-dimethyl-2, 3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate

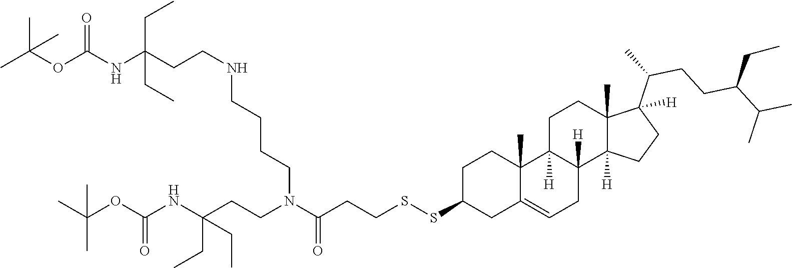

To a stirred solution of 3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoic acid (0.150 g, 0.280 mmol), tert-butyl N-(1-{[4-({3-[(tert-butoxycarbonyl)amino]-3-ethylpentyl}amino)butyl]amino}-3-ethylpentan-3-yl)carbamate (0.217 g, 0.421 mmol), and triethylamine (0.14 mL, 1.0 mmol) in DCM (3.75 mL) was added 50 wt % propanephosphonic acid anhydride in DCM (0.29 mL, 0.56 mmol) dropwise. The reaction mixture stirred at rt and was monitored by LCMS. At 17 h, the reaction mixture was diluted with DCM to 15 mL, then washed with 5% aq. NaHCO₃ solution. The aqueous was extracted with DCM (2×15 mL). The combined organics were dried over Na₂SO₄ and concentrated. The crude material was purified via silica gel chromatography (3:2 EtOAc/hexanes, then 0-20% (5% conc. aq. NH₄OH in MeOH) in DCM) to afford tert-butyl (6,6,17-triethyl-9-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate (0.110 g, 0.107 mmol, 38.2%) as a white foam. UPLC/ELSD: RT=3.01 min. MS (ES): m/z=1032.19 (M+H)⁺ for C₆₀H₁₁₀N₄O₅S₂. ¹H NMR (300 MHz, CDCl₃) δ 5.44-5.32 (m, 1H), 4.89 (br. s, 1H), 4.53-4.16 (m, 1H), 3.43-3.14 (m, 4H), 3.11-2.86 (m, 2H), 2.84-2.43 (m, 5H), 2.43-2.22 (m, 2H), 2.10-0.73 (m, 84H), 0.99 (s, 3H), 0.92 (d, J=6.6 Hz, 3H), 0.67 (s, 3H).

### Step 2: N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride

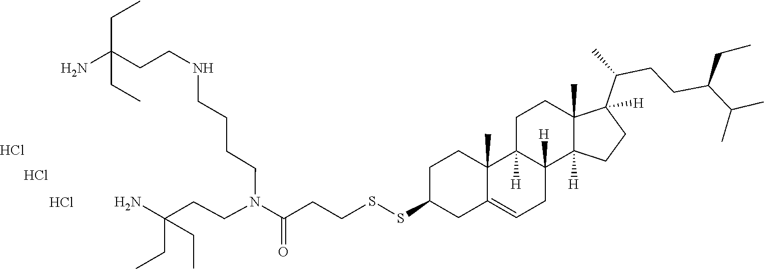

To a stirred solution of tert-butyl (6,6,17-triethyl-9-(3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanoyl)-2,2-dimethyl-4-oxo-3-oxa-5,9,14-triazanonadecan-17-yl)carbamate (0.103 g, 0.100 mmol) in DCM (2.1 mL) was added 4 N HCl in dioxane (0.17 mL, 0.68 mmol). The reaction mixture stirred at rt and was monitored by LCMS. At 16 h, the reaction mixture was diluted with MTBE to 20 mL, then centrifuged (10,000×g for 30 min at 4° C.). The supernatant was drawn off, and the solids were rinsed with MTBE. The solids were suspended in MTBE, then concentrated to afford N-(3-amino-3-ethylpentyl)-N-(4-((3-amino-3-ethylpentyl)amino)butyl)-3-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)disulfaneyl)propanamide trihydrochloride (0.092 g, 0.088 mmol, 87.8%) as a white solid. UPLC/ELSD: RT=2.17 min. MS (ES): m/z=416.72 (M+2H)²⁺ for C₅₀H₉₄N₄OS₂. ¹H NMR (300 MHz, MeOD) δ 5.41-5.33 (m, 1H), 3.58-3.37 (m, 4H), 3.19-3.06 (m, 4H), 3.03-2.92 (m, 2H), 2.88-2.78 (m, 2H), 2.73-2.57 (m, 1H), 2.39-2.27 (m, 2H), 2.20-0.77 (m, 70H), 0.72 (s, 3H).

### CY. Compound SA182: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

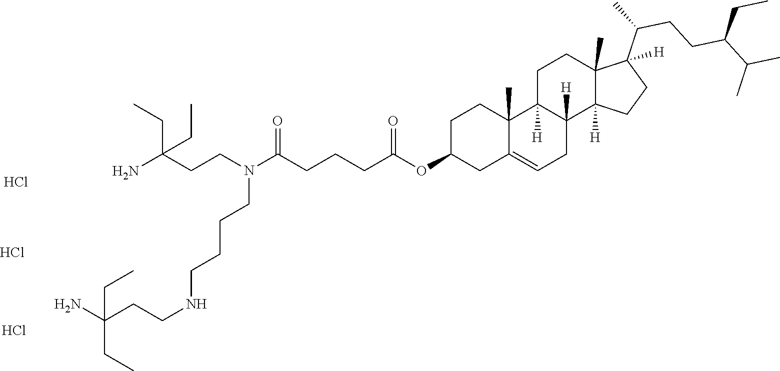

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate

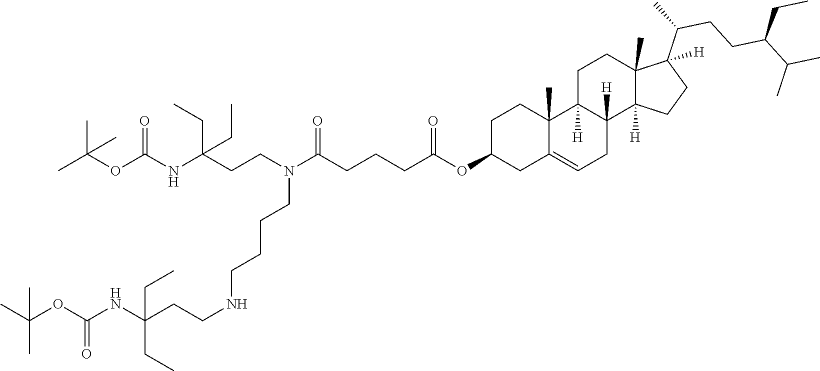

To a stirred solution of 5-(((3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl)oxy)-5-oxopentanoic acid (0.150 g, 0.284 mmol), tert-butyl N-(1-{[4-({3-[(tert-butoxycarbonyl)amino]-3-ethylpentyl}amino)butyl]amino}-3-ethylpentan-3-yl)carbamate (0.219 g, 0.425 mmol), and triethylamine (0.14 mL, 1.0 mmol) in DCM (3.75 mL) was added 50 wt % propanephosphonic acid anhydride in DCM (0.29 mL, 0.56 mmol) dropwise. The reaction mixture stirred at rt and was monitored by LCMS. At 17 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (3:2 EtOAc/hexanes, then 0-20% (5% conc. aq. NH₄OH in MeOH) in DCM) to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.123 g, 0.120 mmol, 42.3%) as a white foam. UPLC/ELSD: RT=2.96 min. MS (ES): m/z=1026.39 (M+H)⁺ for C₆₂H₁₁₂N₄O₇. ¹H NMR (300 MHz, CDCl₃) δ 5.41-5.31 (m, 1H), 5.00-4.70 (m, 1H), 4.69-4.51 (m, 1H), 4.51-4.16 (m, 1H), 3.37-3.11 (m, 4H), 3.11-2.53 (m, 4H), 2.43-2.21 (m, 6H), 2.12-0.72 (m, 84H), 1.01 (s, 3H), 0.92 (d, J=6.4 Hz, 3H), 0.67 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

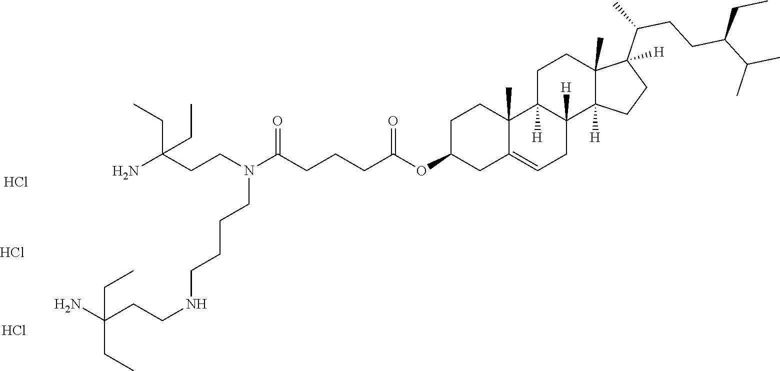

To a solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.119 g, 0.116 mmol) in DCM (2.4 mL) was added 4 N HCl in dioxane (0.20 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off, and the solids were rinsed with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride (0.105 g, 0.092 mmol, 79.5%) as an off-white solid. UPLC/ELSD: RT=2.09 min. MS (ES): m/z=413.39 (M+2H)²⁺ for C₅₂H₉₆N₄O₃. ¹H NMR (300 MHz, MeOD) δ 5.42-5.35 (m, 1H), 4.62-4.46 (m, 1H), 3.57-3.36 (m, 4H), 3.18-3.01 (m, 4H), 2.56-2.25 (m, 6H), 2.20-0.77 (m, 72H), 0.72 (s, 3H).

### CZ. Compound SA183: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

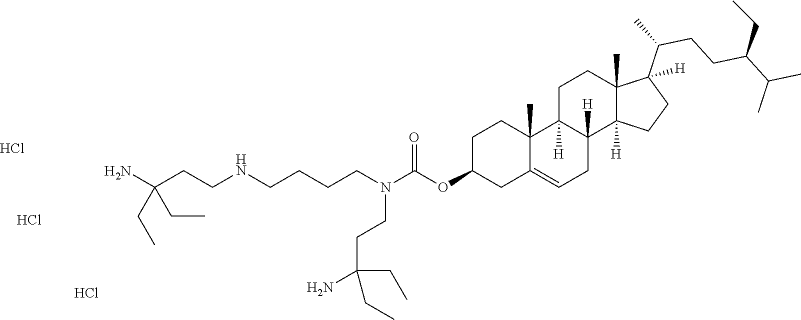

### Step 1: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate

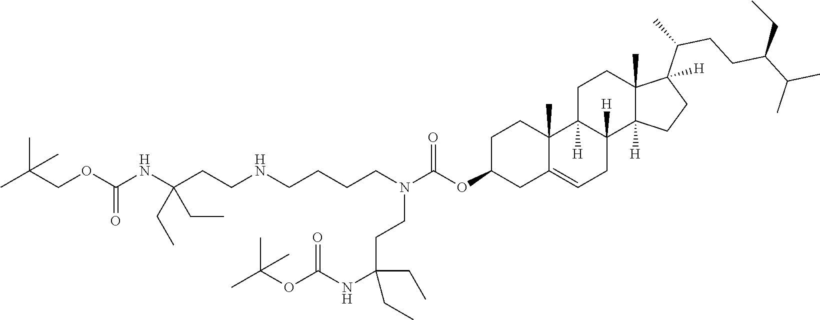

Sitosteryl 4-nitrophenyl carbonate (0.140 g, 0.241 mmol), tert-butyl N-(1-{[4-({3-[(tert-butoxycarbonyl)amino]-3-ethylpentyl}amino)butyl]amino}-3-ethylpentan-3-yl)carbamate (0.187 g, 0.362 mmol), and triethylamine (0.14 mL, 1.0 mmol) were combined in PhMe (3.5 mL). The reaction mixture stirred at 100° C. and was monitored by LCMS. At 16 h, the reaction mixture was cooled to rt, diluted with DCM to 15 mL, and then washed with 5% aq. K₂CO₃ solution. (2×). The combined washes were extracted with DCM (2×15 mL). The combined organics were passed through a hydrophobic frit, dried over Na₂SO₄, and concentrated. The crude material was purified via silica gel chromatography (0-20% (5% conc. aq. NH₄OH in MeOH) in DCM). The material was further purified via silica gel chromatography (1:1 EtOAc/hexanes, then 0-20% (5% conc. aq. NH₄OH in MeOH) in DCM) to afford (3 S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate (0.097 g, 0.10 mmol, 41.5%) as a white foam. UPLC/ELSD: RT=2.95 min. MS (ES): m/z=956.34 (M+H)⁺ for C₅₈H₁₀₆N₄O₆. ¹H NMR (300 MHz, CDCl₃) δ 5.44-5.29 (m, 1H), 5.17-4.91 (m, 1H), 4.59-4.41 (m, 1H), 4.35-4.06 (m, 1H), 3.35-3.02 (m, 4H), 2.70-2.53 (m, 4H), 2.46-2.17 (m, 2H), 2.08-0.73 (m, 82H), 1.01 (s, 3H), 0.92 (d, J=6.5 Hz, 3H), 0.68 (s, 3H).

### Step 2: (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride

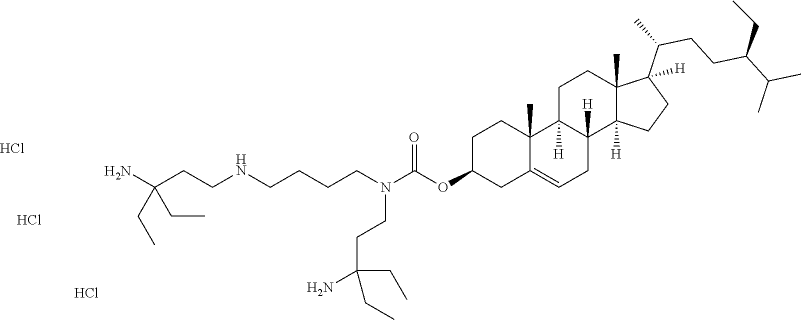

To a stirred solution of (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-((tert-butoxycarbonyl)amino)-3-ethylpentyl)(4-((3-ethyl-3-(((neopentyloxy)carbonyl)amino)pentyl)amino)butyl)carbamate (0.094 g, 0.098 mmol) in DCM (1.9 mL) was added 4 N HCl in dioxane (0.17 mL). The reaction mixture stirred at rt and was monitored by LCMS. At 16 h, the reaction mixture was diluted with MTBE to 20 mL and then centrifuged (10,000×g for 30 min at 4° C.). The supernatant was drawn off, and the solids were rinsed with MTBE. The solids were suspended in MTBE, then concentrated to afford (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-yl (3-amino-3-ethylpentyl)(4-((3-amino-3-ethylpentyl)amino)butyl)carbamate trihydrochloride (0.083 g, 0.089 mmol, 90.1%) as a white solid. UPLC/ELSD: RT=1.99 min. MS (ES): m/z=378.75 (M+2H)²⁺ for C₄₈H₉₀N₄O₂. ¹H NMR (300 MHz, MeOD) δ 5.45-5.36 (m, 1H), 4.52-4.35 (m, 1H), 3.44-3.34 (m, 4H), 3.17-3.05 (m, 4H), 2.42-2.31 (m, 2H), 2.16-0.78 (m, 70H), 0.73 (s, 3H).

### DA. Compound SA184: N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

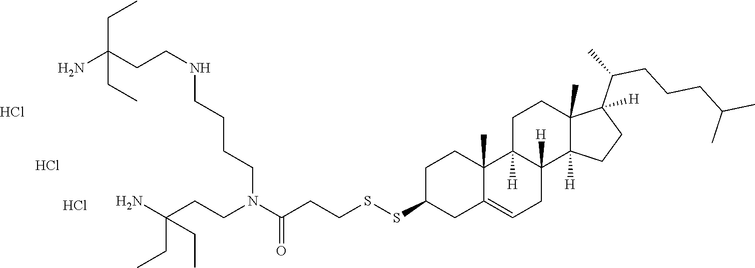

### Step 1: tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate

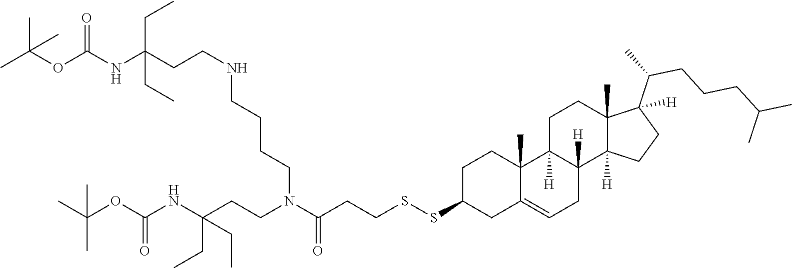

To a stirred solution of 3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoic acid (0.140 g, 0.276 mmol), tert-butyl N-(1-{[4-({3-[(tert-butoxycarbonyl)amino]-3-ethylpentyl}amino)butyl]amino}-3-ethylpentan-3-yl)carbamate (0.213 g, 0.414 mmol), and triethylamine (0.14 mL, 1.0 mmol) in DCM (3.5 mL) was added 50 wt % propanephosphonic acid anhydride in DCM (0.24 mL, 0.468 mmol) dropwise. The reaction mixture stirred at rt and was monitored by LCMS. At 3 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (0-20% (5% conc. aq. NH₄OH in MeOH) in DCM) to afford tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate (0.097 g, 0.097 mmol, 35.1%) as a clear, yellow oil. UPLC/ELSD: RT=2.95 min. MS (ES): m/z=1004.81 (M+H)⁺ for C₅₈H₁₀₆N₄O₅S₂. ¹H NMR (300 MHz, CDCl₃) δ 5.39-5.31 (m, 1H), 5.03-4.74 (m, 1H), 4.52-4.25 (m, 1H), 3.39-3.10 (m, 4H), 3.10-2.85 (m, 3H), 2.85-2.42 (m, 6H), 2.42-2.22 (m, 2H), 2.09-0.72 (m, 78H), 0.99 (s, 3H), 0.91 (d, J=6.5 Hz, 3H), 0.67 (s, 3H).

### Step 2: N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride

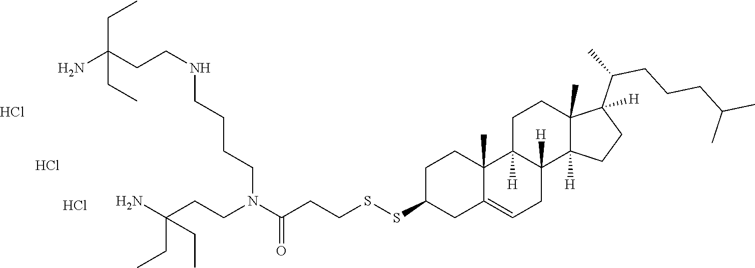

To a stirred solution of tert-butyl (14-(3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanoyl)-6,6,17-triethyl-2,2-dimethyl-4-oxo-3-oxa-5,9,14-triazanonadecan-17-yl)carbamate (0.091 g, 0.091 mmol) in DCM (2.3 mL) was added 4 N HCl in dioxane (0.16 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off. The solids were rinsed with MTBE, then suspended in MTBE and concentrated to afford N-(3-amino-3-ethylpentyl)-N-(4-((3-amino-3-ethylpentyl)amino)butyl)-3-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)disulfaneyl)propanamide trihydrochloride (0.080 g, 0.080 mmol, 88.4%) as a white solid. UPLC/ELSD: RT=2.04 min. MS (ES): m/z=402.54 (M+2H)²⁺ for C₄₈H₉₀N₄OS₂. ¹H NMR (300 MHz, MeOD) δ 5.45-5.31 (m, 1H), 3.58-3.36 (m, 4H), 3.19-3.04 (m, 4H), 3.03-2.89 (m, 2H), 2.89-2.76 (m, 2H), 2.73-2.55 (m, 1H), 2.42-2.23 (m, 2H), 2.18-0.96 (m, 57H), 0.94 (d, J=6.5 Hz, 3H), 0.88 (d, J=6.6 Hz, 6H), 0.72 (s, 3H).

### DB. Compound SA185: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

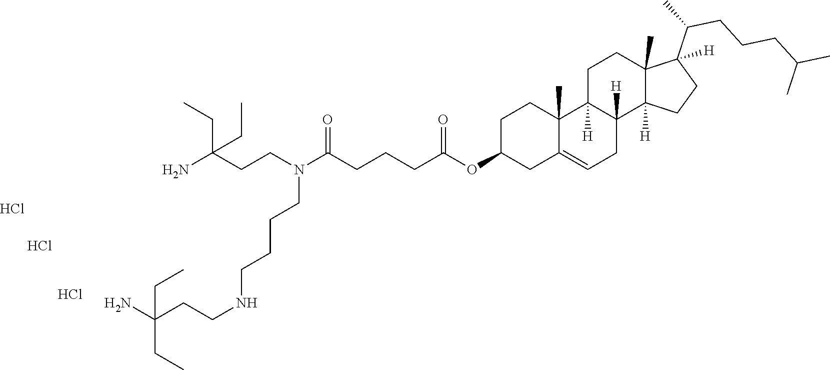

### Step 1: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate

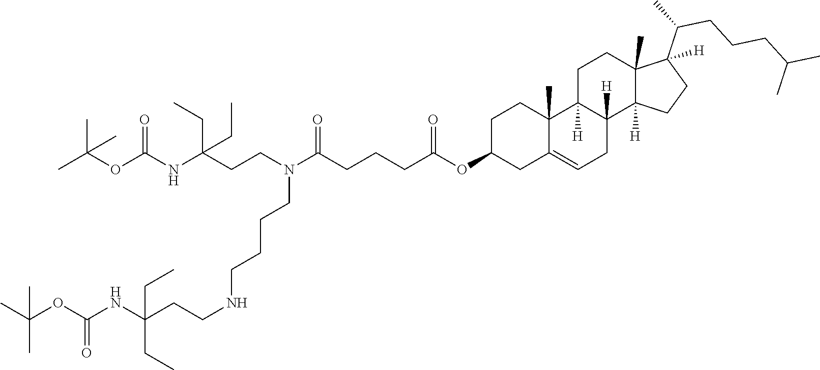

To a stirred solution of 5-(((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)-5-oxopentanoic acid (0.140 g, 0.28 mmol), tert-butyl N-(1-{[4-({3-[(tert-butoxycarbonyl)amino]-3-ethylpentyl}amino)butyl]amino}-3-ethylpentan-3-yl)carbamate (0.216 g, 0.419 mmol), and triethylamine (0.14 mL, 1.0 mmol) in DCM (3.5 mL) was added 50 wt % propanephosphonic acid anhydride in DCM (0.28 mL, 0.546 mmol) dropwise. The reaction mixture stirred at rt and was monitored by LCMS. At 3 h, the reaction mixture was diluted with DCM to 15 mL, then washed with 5% aq. NaHCO₃ solution. The aqueous was extracted with DCM (2×15 mL). The combined organics were passed through a hydrophobic frit, dried over Na₂SO₄, and concentrated. The crude material was purified via silica gel chromatography (0-14% (5% conc. aq. NH₄OH in MeOH) in DCM) to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.071 g, 0.071 mmol, 25.4%) as a clear, yellow oil. UPLC/ELSD: RT=2.89 min. MS (ES): m/z=998.15 (M+H)⁺ for C₆₀H₁₀₈N₄O₇. ¹H NMR (300 MHz, CDCl₃) δ 5.43-5.31 (m, 1H), 5.11-4.87 (m, 1H), 4.68-4.51 (m, 1H), 4.40-4.12 (m, 1H), 3.37-3.12 (m, 4H), 2.72-2.51 (m, 4H), 2.43-2.21 (m, 6H), 2.07-0.74 (m, 80H), 1.01 (s, 3H), 0.91 (d, J=6.5 Hz, 3H), 0.67 (s, 3H).

### Step 2: (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride

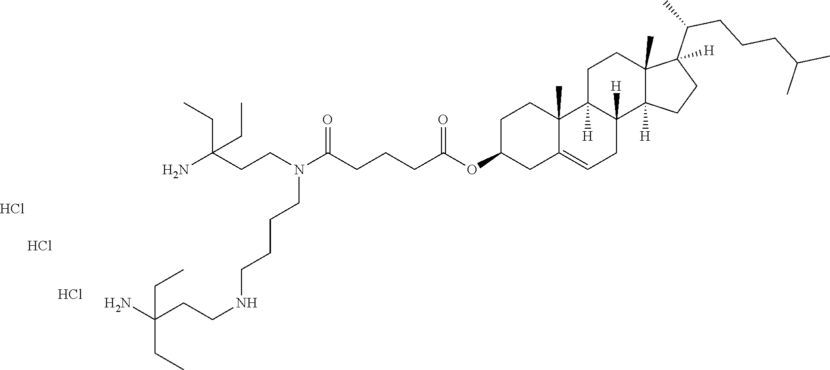

To a stirred solution of (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 14-(3-((tert-butoxycarbonyl)amino)-3-ethylpentyl)-6,6-diethyl-2,2-dimethyl-4,15-dioxo-3-oxa-5,9,14-triazanonadecan-19-oate (0.067 g, 0.067 mmol) in DCM (1.8 mL) was added 4 N HCl in dioxane (0.12 mL). The reaction mixture stirred at rt and was monitored by LCMS. At 16 h, the reaction mixture was diluted with MTBE to 20 mL, then centrifuged (10,000×g for 30 min at 4° C.). The supernatant was drawn off. The solids were rinsed with MTBE, then suspended in MTBE and concentrated to afford (3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 5-((3-amino-3-ethylpentyl)(4-((3-amino-3-ethylpentyl)amino)butyl)amino)-5-oxopentanoate trihydrochloride (0.061 g, 0.066 mmol, 97.5%) as a white solid. UPLC/ELSD: RT=1.96 min. MS (ES): m/z=399.58 (M+2H)²⁺ for C₅₀H₉₂N₄O₃. ¹H NMR (300 MHz, MeOD) δ 5.45-5.33 (m, 1H), 4.62-4.46 (m, 1H), 3.56-3.36 (m, 4H), 3.18-3.03 (m, 4H), 2.55-2.25 (m, 6H), 2.18-0.98 (m, 59H), 0.95 (d, J=6.5 Hz, 3H), 0.88 (d, J=6.6 Hz, 6H), 0.73 (s, 3H).

### Example 2

### Production of Nanoparticle Compositions

Lipids are dissolved in ethanol at a concentration of 24 mg/mL and molar ratios of 49.0:11.2:39.3:0.5 (IL1:DSPC:cholesterol:PEG-DMG-2K) and mixed with the acidification buffer (45 mM acetate buffer at pH 4). The lipid solution and acidification buffer are mixed using a multi-inlet vortex mixer at a 3:7 volumetric ratio of lipid:buffer for mixer 1 and mixer 2 and a 1:3 volumetric ratio of lipid:buffer (25% ethanol) for mixer 3. After a 5 second residence time, the resulting nanoparticles are mixed with 55 mM sodium acetate at pH 5.6 at a volumetric ratio of 5:7 of nanoparticle:buffer. See Table 2a for mixing parameters. The resulting dilute nanoparticles are then buffer exchanged and concentrated using tangential flow filtration (TFF) into a final buffer containing 5 mM sodium acetate pH 5.0. See Table 2b for TFF parameters. Then a 70% sucrose solution in 5 mM acetate buffer at pH 5 is subsequently added.

TABLE 2a
Mixing Parameters

| | Mixer | | |
|---|---|---|---|
| Stream | 1 | 2 | 3 |
| 1 mL/min | 21 | 75 | 281 |
| 2 mL/min | 49 | 175 | 844 |
| 3 mL/min | 98 | 350 | 1575 |
| Nano-precipitation | 30% | 30% | 25% |
| (1 + 2) | | | |
| Mixed product | 12.5% | 12.5% | 10.4% |
| (1 + 2 + 3) | | | |

TABLE 2b
TFF Parameters

| TFF Parameter | Mixer Value | |
|---|---|---|
| Feed flux | 240 | L/m²/hr |
| Loading | 100-300 | g/m³ |
| Initial concentration: Factor | 10x | |
| Diavolumes (DVs) | 8 | DVs |
| Final concentration: Factor | 4x | |
| TMP | 4-6 | psi |

The resulting nanoparticles at a lipid concentration of 7.33 mg/mL in 5 mM acetate (pH 5) and 75 g/L sucrose are mixed with mRNA (luciferease or CFTR) at a concentration of 0.625 mg/mL in 42.5 mM sodium acetate pH 5.0, with N:P of 4.93. The nanoparticle solution and nanoparticles are mixed using a multi-inlet vortex mixer at a 3:2 volumetric ratio of nanoparticle:mRNA. Once loaded with mRNA, these intermediate nanoparticles undergo a 300 second residence time prior addition of neutralization buffer containing 120 mM TRIS pH 8.12 at a volumetric ratio of 5:1 of nanoparticle:buffer.

For HeLa studies evaluating luciferase protein expression, PEG-DMG-2K dissolved in a 20 mM TRIS buffer (pH 7.5) is added to the neutralized intermediate nanoparticle solution at a ratio of 1:6, bringing the solution to the final molar ratios of IL1:DSPC:cholesterol:PEG-DMG-2K of 48.5:11.1:38.9:1.5%. This nanoparticle formulation is then modified with lipid amines. In a typical example, nanoparticle formulation at a concentration of 0.18 mg/mL mRNA and a 0.56 mL volume is modified with lipid amine SA50 (467.2 nmol) prepared in buffer containing 20 mM TRIS, 14.3 mM sodium acetate, 32 g/L sucrose and 140 mM NaCl-pH 7.5 at a volumetric ratio of 1:1 of nanoparticle:buffer.

For HeLa studies evaluating CFTR protein expression, the intermediate nanoparticle formulation (1 mL, 0.415 mg mRNA) is mixed with a buffer containing 20 mM TRIS (pH 7.5), 0.9 mg/mL PEG-DMG-2K and lipid amine SA50 (647.1 nmol) at a volumetric ratio of 6:1 of nanoparticle:buffer. The resulting nanoparticle suspension undergoes concentration using a centrifugal filtration device (100 kDa molecular weight cut-off) and is diluted in running buffer (20 mM TRIS, 14.3 mM sodium acetate, and 32 g/L sucrose, pH 7.5) with a 300 mM NaCl solution to a final buffer matrix containing 70 mM NaCl.

Both resulting nanoparticle suspensions are filtered through a 0.8/0.2 μm capsule filter and filled into glass vials at an mRNA strength of about 0.1-1 mg/mLBliophysical data (Diameter and PDI from DLS measurements and Encapsualtion using Ribogreen assay) for both luciferase and CFTR mRNA nanoparticles with lipid amines is shown in Tables 2c and d, respectively.

TABLE 2c
Luciferase mRNA nanoparticle biophysical data

| | Diameter | | % Encapsulation |
|---|---|---|---|
| SA # | (nm) | PDI | (Ribogreen) |
| SA59 | 74 | 0.15 | 99.7 |
| SA60 | 74 | 0.13 | 98.8 |
| SA61 | 77 | 0.16 | 99.1 |
| SA62 | 78 | 0.20 | 99.0 |
| SA66 | 72 | 0.18 | 88.2 |
| SA69 | 71 | 0.16 | 98.9 |
| SA70 | 76 | 0.21 | 97.9 |
| SA71 | 73 | 0.15 | 99.1 |
| SA72 | 72 | 0.11 | 99.1 |
| SA74 | 75 | 0.19 | 99.1 |
| SA75 | 74 | 0.16 | 98.8 |
| SA76 | 75 | 0.20 | 98.1 |
| SA77 | 74 | 0.20 | 98.2 |
| SA78 | 76 | 0.20 | 99.8 |
| SA79 | 74 | 0.18 | 97.0 |
| SA81 | 72 | 0.12 | 99.8 |
| SA82 | 73 | 0.11 | 99.1 |
| SA83 | 75 | 0.14 | 99.3 |
| SA84 | 74 | 0.16 | 99.9 |
| SA87 | 75 | 0.18 | 98.2 |
| SA88 | 75 | 0.13 | 99.8 |
| SA89 | 77 | 0.16 | 99.7 |
| SA90 | 76 | 0.19 | 99.6 |
| SA95 | 75 | 0.20 | 99.7 |
| SA96 | 71 | 0.15 | 99.6 |
| SA97 | 72 | 0.16 | 98.8 |
| SA98 | 70 | 0.15 | 97.0 |
| SA110 | 70 | 0.14 | 98.8 |
| SA111 | 76 | 0.18 | 99.6 |
| SA113 | 77 | 0.23 | 99.7 |
| SA114 | 75 | 0.18 | 99.7 |
| SA116 | 73 | 0.20 | 97.6 |
| SA117 | 77 | 0.28 | 99.7 |
| SA118 | 74 | 0.19 | 99.7 |
| SA119 | 78 | 0.18 | 99.4 |
| SA120 | 76 | 0.28 | 99.1 |
| SA122 | 75 | 0.15 | 99.4 |
| SA123 | 73 | 0.14 | 99.5 |
| SA124 | 74 | 0.14 | 99.8 |
| SA125 | 77 | 0.19 | 99.7 |
| SA126 | 75 | 0.18 | 99.7 |
| SA127 | 74 | 0.17 | 99.7 |
| SA128 | 75 | 0.16 | 99.4 |
| SA129 | 70 | 0.16 | 99.5 |
| SA131 | 72 | 0.12 | 99.6 |
| SA132 | 81 | 0.41 | 99.3 |
| SA133 | 73 | 0.17 | 99.0 |
| SA134 | 72 | 0.13 | 99.5 |
| SA135 | 71 | 0.12 | 99.3 |
| SA136 | 75 | 0.16 | 99.7 |
| SA137 | 79 | 0.20 | 99.6 |
| SA138 | 72 | 0.19 | 99.2 |
| SA139 | 72 | 0.12 | 99.4 |
| SA141 | 73 | 0.15 | 98.9 |
| SA142 | 72 | 0.19 | 99.5 |
| SA144 | 71 | 0.15 | 99.2 |
| SA145 | 77 | 0.17 | 99.7 |
| SA149 | 75 | 0.15 | 99.5 |
| SA151 | 72 | 0.12 | 95.8 |
| SA152 | 78 | 0.15 | 98.2 |
| SA153 | 72 | 0.15 | 95.8 |
| SA154 | 73 | 0.18 | 99.6 |
| SA155 | 73 | 0.11 | 98.9 |
| SA156 | 74 | 0.15 | 99.5 |
| SA157 | 75 | 0.13 | 99.6 |
| SA158 | 79 | 0.28 | 99.7 |
| SA160 | 75 | 0.11 | 99.7 |
| SA161 | 77 | 0.16 | 99.6 |
| SA162 | 75 | 0.15 | 99.4 |
| SA163 | 71 | 0.17 | 99.1 |
| SA164 | 73 | 0.14 | 98.0 |
| SA165 | 72 | 0.13 | 98.8 |
| SA166 | 75 | 0.14 | 99.2 |
| SA167 | 75 | 0.15 | 98.7 |
| SA168 | 79 | 0.16 | 99.3 |
| SA169 | 77 | 0.20 | 99.4 |
| SA170 | 75 | 0.21 | 99.6 |
| SA171 | 73 | 0.23 | 99.2 |
| SA172 | 74 | 0.10 | 99.4 |
| SA173 | 73 | 0.18 | 99.5 |
| SA174 | 73 | 0.17 | 99.5 |
| SA178 | 75 | 0.20 | 98.8 |
| SA179 | 73 | 0.17 | 99.0 |
| SA180 | 76 | 0.17 | 98.7 |
| SA181 | 74 | 0.16 | 99.5 |
| SA182 | 73 | 0.14 | 99.0 |
| SA183 | 76 | 0.21 | 99.6 |
| SA184 | 74 | 0.12 | 98.8 |
| SA185 | 75 | 0.16 | 99.4 |

TABLE 2d
CFTR mRNA nanoparticle biophysical data

| | Diameter | | % Encapsulation |
|---|---|---|---|
| SA# | (nm) | PDI | (Ribogreen) |
| SA50 | 77.1 | 0.11 | 99.3 |
| SA51 | 74.3 | 0.15 | 98.4 |
| SA58 | 71.6 | 0.10 | 99.6 |
| SA59 | 76.9 | 0.15 | 99.6 |
| SA60 | 70.9 | 0.08 | 99.5 |
| SA61 | 71.3 | 0.11 | 99.4 |
| SA62 | 74.2 | 0.10 | 99.6 |
| SA64 | 67.8 | 0.09 | 99.1 |
| SA65 | 78.0 | 0.14 | 99.6 |
| SA66 | 75.7 | 0.13 | 99.5 |
| SA67 | 72.8 | 0.10 | 99.2 |
| SA68 | 74.6 | 0.12 | 99.2 |
| SA69 | 74.0 | 0.13 | 99.9 |
| SA70 | 76.2 | 0.16 | 99.8 |
| SA71 | 74.3 | 0.12 | 99.8 |
| SA72 | 73.0 | 0.11 | 99.8 |
| SA73 | 92.8 | 0.16 | 100 |
| SA75 | 86.9 | 0.11 | 100 |
| SA76 | 83.9 | 0.11 | 100 |
| SA77 | 83.9 | 0.13 | 100 |
| SA78 | 87.7 | 0.17 | 100 |
| SA79 | 78.4 | 0.13 | 97.9 |
| SA81 | 87.6 | 0.18 | 99.4 |
| SA82 | 80.7 | 0.13 | 99.4 |
| SA83 | 82.3 | 0.15 | 99.8 |
| SA84 | 82 | 0.13 | 99.5 |
| SA87 | 81.2 | 0.1 | 99.5 |
| SA88 | 89.4 | 0.19 | 99.1 |
| SA89 | 86.5 | 0.15 | 98.6 |
| SA90 | 84.6 | 0.14 | 98.1 |
| SA95 | 83.6 | 0.14 | 99.3 |
| SA96 | 82.3 | 0.12 | 98.9 |
| SA97 | 83.2 | 0.12 | 98.4 |
| SA98 | 82.8 | 0.14 | 93 |
| SA110 | 84.2 | 0.15 | 98.8 |
| SA111 | 86.4 | 0.14 | 98.6 |
| SA114 | 85.9 | 0.13 | 98.5 |
| SA117 | 94.7 | 0.17 | 98.6 |
| SA118 | 89.9 | 0.16 | 97.9 |
| SA119 | 88.3 | 0.15 | 98.9 |
| SA120 | 83.6 | 0.14 | 97.5 |
| SA121 | 79.2 | 0.14 | 97.4 |
| SA122 | 83.5 | 0.13 | 98.6 |
| SA123 | 83.2 | 0.12 | 99.0 |
| SA124 | 72.0 | 0.10 | 98.3 |
| SA125 | 75.0 | 0.15 | 99.0 |
| SA126 | 79.3 | 0.15 | 99.4 |
| SA127 | 74.1 | 0.12 | 99.5 |
| SA128 | 74.5 | 0.13 | 99.4 |
| SA129 | 81.6 | 0.12 | 96.2 |
| SA130 | 85.7 | 0.18 | 95.7 |
| SA131 | 83.8 | 0.14 | 98.6 |
| SA132 | 84.2 | 0.15 | 96.8 |
| SA133 | 73.5 | 0.11 | 99.4 |
| SA134 | 74.1 | 0.10 | 98.7 |
| SA135 | 73.5 | 0.11 | 98.0 |
| SA136 | 76.9 | 0.13 | 97.7 |
| SA137 | 76.3 | 0.13 | 98.3 |
| SA138 | 89.6 | 0.12 | 96.4 |
| SA139 | 68.9 | 0.12 | 99.4 |
| SA141 | 70.8 | 0.12 | 98.9 |
| SA142 | 69.5 | 0.14 | 99.0 |
| SA144 | 69.3 | 0.12 | 98.8 |
| SA145 | 71.8 | 0.14 | 99.8 |
| SA149 | 72.3 | 0.14 | 99.9 |
| SA151 | 72.0 | 0.14 | 98.2 |
| SA152 | 71.6 | 0.14 | 98.4 |
| SA153 | 69.3 | 0.12 | 98.5 |
| SA154 | 72.8 | 0.13 | 99.1 |
| SA155 | 71.1 | 0.13 | 99.4 |
| SA156 | 76.5 | 0.18 | 99.6 |
| SA157 | 73.9 | 0.16 | 99.8 |
| SA158 | 72.4 | 0.15 | 98.3 |
| SA160 | 77.4 | 0.15 | 98.1 |
| SA161 | 74.2 | 0.15 | 99.6 |
| SA162 | 75.4 | 0.16 | 98.6 |
| SA163 | 70.6 | 0.09 | 100 |
| SA164 | 72.5 | 0.14 | 99.2 |
| SA165 | 69.8 | 0.12 | 98.8 |
| SA166 | 76.1 | 0.16 | 99.5 |
| SA167 | 75.1 | 0.16 | 100 |
| SA168 | 76.1 | 0.15 | 99.3 |
| SA169 | 73.0 | 0.14 | 99.2 |
| SA170 | 74.8 | 0.15 | 99.2 |
| SA171 | 71.3 | 0.11 | 99.4 |
| SA172 | 70.3 | 0.13 | 98.3 |
| SA173 | 71.8 | 0.16 | 100 |
| SA174 | 71.1 | 0.16 | 98.1 |
| SA178 | 83 | 0.26 | 100 |
| SA179 | 73 | 0.15 | 99.8 |
| SA181 | 69 | 0.13 | 98.7 |
| SA182 | 72 | 0.13 | 98.0 |
| SA183 | 71 | 0.14 | 99.0 |
| SA184 | 71 | 0.13 | 99.0 |
| SA185 | 73 | 0.16 | 98.6 |

### Example 3

### Protein Expression in Human Cervical Cancer Epithelial Cell (HeLa)

Lipid nanoparticle compositions were prepared in a manner analogous to that in example 2. To evaluate LNP cellular uptake and protein expression In Vitro, HeLa cells from ATCC.org (ATCC CCL-2) are used. The cells are cultured in complete Minimum Essential Medium (MVEM) and are plated in 96 well Cell Carrier Ultra plate with PDL coated surface (PerkinElmer) prior to running an experiment.

### Luciferase Protein Expression Assay in HeLa Cells

Cells were transfected with buffer control (PBS) or LNPs encapsulating Luciferase mRNA (25 ng per well; N=4 replicate wells) in serum-free MEM media. LNP transfected cells were incubated for 5 h, followed by media removal and supplementation with complete MEM media. Cells were further incubated in complete MEM media overnight (24 h). Following the 24 hr incubation, luciferase protein expression was measured using the ONE-Glo™ Luciferase Assay (Promega). Cells were lysed using 1× Passive Lysis Buffer (Cat. #E194A) for 10 min in a microplate mixer at room temperature. Luciferase in the supernatant was measured by adding Luciferase Assay Reagent (Cat. #E151A) containing luciferin. Bioluminescence was then immediately measured on a Synergy H₁ plate reader (BioTek). The results shown in Table 3a show the Average Relative Light Units (RLU) of each sample tested.

TABLE 3a
Luciferase Expression Results

| | Average Relative Light Units ± |
|---|---|
| SA # | Standard Deviation |
| SA59 | 17590.1 ± 6267.4 |
| SA60 | 30508.5 ± 6869.7 |
| SA61 | 14343.1 ± 5298.1 |
| SA62 | 18807.1 ± 6618.4 |
| SA66 | 13951.3 ± 6740.3 |
| SA69 | 35256.9 ± 8309.9 |
| SA70 | 11822.1 ± 4128.6 |
| SA71 | 32758.9 ± 5897.1 |
| SA72 | 30826.5 ± 5913.2 |
| SA74 | 23899.2 ± 6943.7 |
| SA75 | 37732.4 ± 6382.7 |
| SA76 | 38232.8 ± 4603.5 |
| SA77 | 39191.9 ± 4319 |
| SA78 | 14341.8 ± 3497.6 |
| SA79 | 105.1 ± 20 |
| SA81 | 19619.8 ± 4362.6 |
| SA82 | 33396.2 ± 4902.7 |
| SA83 | 35394.8 ± 5033.2 |
| SA84 | 29639.9 ± 7554 |
| SA87 | 13346.1 ± 2590.8 |
| SA88 | 17105.3 ± 7097.3 |
| SA89 | 8209.2 ± 4138.5 |
| SA90 | 9337.4 ± 3055.2 |
| SA95 | 22952.2 ± 5937.7 |
| SA96 | 17172.8 ± 2176 |
| SA97 | 13144.4 ± 4350.5 |
| SA98 | 80.7 ± 36.7 |
| SA110 | 14227 ± 2513.9 |
| SA111 | 26647.6 ± 9522.1 |
| SA113 | 20665.4 ± 7510.5 |
| SA114 | 22594.3 ± 9128.4 |
| SA116 | 110.9 ± 26.9 |
| SA117 | 10134.1 ± 3860.2 |
| SA118 | 12261.7 ± 4328.5 |
| SA119 | 18979.6 ± 7864.8 |
| SA120 | 34695.5 ± 9492.2 |
| SA122 | 29605.6 ± 6326.9 |
| SA123 | 28520.5 ± 6429.4 |
| SA124 | 45805.9 ± 5630.6 |
| SA125 | 15625.8 ± 3399.4 |
| SA126 | 31706.7 ± 7386.3 |
| SA127 | 41246.9 ± 5672.4 |
| SA128 | 39386.4 ± 5261.4 |
| SA129 | 144.8 ± 21.6 |
| SA131 | 40915.5 ± 4983.3 |
| SA132 | 131.6 ± 15.5 |
| SA133 | 26696.2 ± 5831.4 |
| SA134 | 29194.3 ± 6336.9 |
| SA135 | 27499.8 ± 7269.5 |
| SA136 | 16245.8 ± 6554.6 |
| SA137 | 15239.8 ± 6248.1 |
| SA138 | 22039.8 ± 8550.1 |
| SA139 | 24497.9 ± 3998.9 |
| SA141 | 35801.9 ± 8884.3 |
| SA142 | 44925.4 ± 6006.5 |
| SA144 | 33357.5 ± 5288.8 |
| SA145 | 36033.7 ± 4254.5 |
| SA149 | 31035.2 ± 6333.7 |
| SA151 | 36345.5 ± 6730.7 |
| SA152 | 41692.8 ± 9336.8 |
| SA153 | 35859.3 ± 5894.9 |
| SA154 | 28128.6 ± 4655.3 |
| SA155 | 31922.9 ± 8053.8 |
| SA156 | 37662.9 ± 8258.6 |
| SA157 | 24130.4 ± 4741.7 |
| SA158 | 33097.8 ± 7405.1 |
| SA160 | 19694.4 ± 6954.5 |
| SA161 | 17839.3 ± 7760 |
| SA162 | 34454.6 ± 3101.8 |
| SA163 | 30168 ± 7878.9 |
| SA164 | 17129.2 ± 2374.1 |
| SA165 | 33585.6 ± 7766.5 |
| SA166 | 33377.3 ± 8879.8 |
| SA167 | 38742.4 ± 6037.5 |
| SA168 | 35639.5 ± 6688.9 |
| SA169 | 28034.9 ± 4050.3 |
| SA170 | 23687.3 ± 3865 |
| SA171 | 22232.4 ± 9084.1 |
| SA172 | 27528.4 ± 6654.4 |
| SA173 | 33596.1 ± 6033 |
| SA174 | 31280.7 ± 5830.7 |
| SA178 | 10196.5 ± 602.4 |
| SA179 | 12298.9 ± 928.6 |
| SA180 | 35476 ± 6770.8 |
| SA181 | 12107.3 ± 1481.4 |
| SA182 | 38564.9 ± 12453 |
| SA183 | 33915.2 ± 6639.9 |
| SA184 | 26222.4 ± 4612.1 |
| SA185 | 36168.3 ± 7638.9 |

### CFTR Protein Expression Assay in HeLa Cells

Buffer control (PBS) and LNPs encapsulating cystic fibrosis transmembrane conductance regulator (CFTR) mRNA were dosed with MEM media in the absence of serum (N=4 replicate wells). LNP transfected cells were incubated for 5 h, followed by media removal and supplementation with complete MEM media. Cells were further incubated in complete MEM media overnight (24 h).

Following 24 hr incubation, the cells were fixed with PFA and processed for immunofluorescence (IF) using an anti-CFTR rabbit monoclonal antibody. Briefly, the cells were permeabilized with 0.5% TX-100 for 10 min, blocked with 3% bovine serum albumin (BSA)+PBST for 1 hr at room temperature, and incubated with primary anti-CFTR monoclonal antibody overnight at 4° C. Following primary antibody incubation, the cells were incubated with Alexa 488 conjugated secondary antibody for 30 mi and stained with DAPI and HCS CellMask Blue stain. Between different incubation steps the cells were either washed with PBS or PBST. Cells were imaged using Opera Phoenix spinning disk confocal microscope (PerkinElmer), and CFTR protein expression was detected using the 488 nm channel. The image analysis was performed in Harmony 4.9, with main analysis output being CFTR intensity per cell. The results shown in Table 3b show fold-change in CFTR signal intensity per cell compared to buffer (PBS) control (NA=not available).

TABLE 3b
CFTR Protein Expression Results

| | Average fold-change in CFTR |
|---|---|
| | signal intensity per |
| Lipid | cell compared to buffer |
| amine | control ± standard deviation |
| SA50 | 7.1 ± 0.4 |
| SA51 | 6.5 ± 0.4 |
| SA58 | 4.5 ± 0.3 |
| SA59 | 5.6 ± 0.5 |
| SA60 | 10.7 ± 0.1 |
| SA61 | 7.2 ± 0.4 |
| SA62 | 5.5 ± 1.3 |
| SA64 | 9.1 ± 0.6 |
| SA65 | 5.9 ± 0.5 |
| SA66 | 2.4 ± 0.1 |
| SA67 | 7.7 ± 0.7 |
| SA68 | 8.9 ± 0.4 |
| SA69 | 5.3 ± 0.5 |
| SA70 | 4.5 ± 0.8 |
| SA71 | 6.0 ± 0.4 |
| SA72 | 6.6 ± 0.6 |
| SA73 | 9.4 ± 0.6 |
| SA75 | 11.9 ± 0.6 |
| SA76 | 12.0 ± 1.1 |
| SA77 | 12.2 ± 1.3 |
| SA78 | 11.3 ± 0.7 |
| SA79 | 0.8 ± 0 |
| SA81 | 4.4 ± 0.3 |
| SA82 | 4.6 ± 0.2 |
| SA83 | 4.8 ± 0.3 |
| SA84 | 5.0 ± 0.2 |
| SA87 | 3.2 ± 0.1 |
| SA88 | 4.5 ± 0.2 |
| SA89 | 3.7 ± 0.4 |
| SA90 | 4.2 ± 0.4 |
| SA95 | 4.2 ± 0.8 |
| SA96 | 5.2 ± 0.7 |
| SA97 | 5.3 ± 1.0 |
| SA98 | 1.0 ± 0 |
| SA110 | 4.0 ± 0.2 |
| SA111 | 4.8 ± 0.2 |
| SA114 | 6.3 ± 1.3 |
| SA117 | 4.9 ± 1.2 |
| SA118 | 9.7 ± 1.2 |
| SA119 | 5.7 ± 1.0 |
| SA120 | 16.8 ± 1.1 |
| SA121 | 16.2 ± 1.8 |
| SA122 | 14.8 ± 1.1 |
| SA123 | 14.7 ± 1.0 |
| SA124 | 15.0 ± 1.2 |
| SA125 | 14.2 ± 0.9 |
| SA126 | 18.2 ± 2.0 |
| SA127 | 20.1 ± 1.9 |
| SA128 | 19.9 ± 0.4 |
| SA129 | 1.0 ± 0 |
| SA130 | 1.0 ± 0 |
| SA131 | 10.8 ± 0.5 |
| SA132 | 1.6 ± 0.1 |
| SA133 | 18.5 ± 0.8 |
| SA134 | 17.7 ± 0.9 |
| SA135 | 16.6 ± 1.5 |
| SA136 | 15.8 ± 1.4 |
| SA137 | 14.0 ± 0.8 |
| SA138 | 12.1 ± 1.1 |
| SA139 | 13.3 ± 1.2 |
| SA141 | 14.9 ± 0.6 |
| SA142 | 20.0 ± 1.0 |
| SA144 | 17.9 ± 2.1 |
| SA145 | 14.4 ± 0.6 |
| SA149 | 15.8 ± 1.4 |
| SA151 | 20.2 ± 1.3 |
| SA152 | 20.6 ± 1.6 |
| SA153 | 17.5 ± 1.4 |
| SA154 | 16.7 ± 0.8 |
| SA155 | 12.2 ± 1.0 |
| SA156 | 14.6 ± 0.8 |
| SA157 | 15.9 ± 1.4 |
| SA158 | 20.4 ± 1.3 |
| SA160 | 18.2 ± 1.4 |
| SA161 | 14.1 ± 1.5 |
| SA162 | 21.3 ± 0.6 |
| SA163 | 11.3 ± 0.5 |
| SA164 | 16.2 ± 0.7 |
| SA165 | 14.0 ± 0.5 |
| SA166 | 15.6 ± 1.3 |
| SA167 | 14.9 ± 1.4 |
| SA168 | 14.8 ± 0.7 |
| SA169 | 19.7 ± 1.6 |
| SA170 | 19.0 ± 1.7 |
| SA171 | 18.9 ± 1.9 |
| SA172 | 17.8 ± 1.1 |
| SA173 | 13.5 ± 0.8 |
| SA174 | 14.8 ± 1.4 |
| SA178 | 9.8 ± 0.3 |
| SA179 | 7.0 ± 0.6 |
| SA181 | 6.3 ± 0.4 |
| SA182 | 13.2 ± 0.9 |
| SA183 | 13.6 ± 0.7 |
| SA184 | 11.3 ± 0.7 |
| SA185 | 14.2 ± 1.0 |

A number of embodiments have been described. Nevertheless, it will be understood that various modifications may be made without departing from the spirit and scope of what has been described. Accordingly, other embodiments are within the scope of the following claims. Each reference, including all patent, patent applications, and publications, cited in the present application is incorporated herein by reference in its entirety.

What is claimed is:

1. A lipid amine compound of Formula A1: 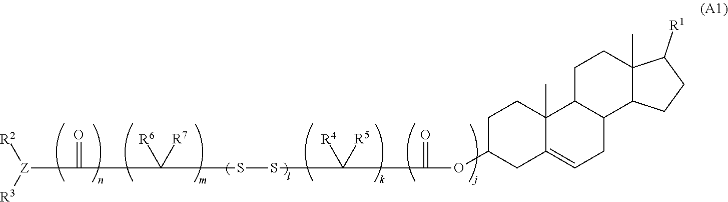
or a salt thereof, wherein:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₂₋₂₀ alkyl are optionally replaced with O;
(iii) 1, 2, 3, or 4 non-terminal carbons of the C₂₋₂₀ alkyl are optionally replaced with NR¹⁰;
(iv) 1, 2, 3, or 4 non-terminal carbons of the C₂₋₂₀ alkyl are optionally replaced with C(═O); and
(v) 1, 2, 3, or 4 non-terminal carbons of the C₂₋₂₀ alkyl are optionally replaced with CR^{a}R^{b} wherein R^{a} and R^{b} together with the C atom to which they are attached form a C₃₋₆ cycloalkyl group;
wherein R² and R³ are the same or different;
or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo;
or R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo;
R⁴, R⁵, R⁶, and R⁷ are each independently selected from H, halo, and C₁₋₄ alkyl;
or R⁴ and R⁵ together with the carbon atom to which they are attached form a C₃₋₇ cycloalkyl group;
or R⁶ and R⁷ together with the carbon atom to which they are attached form a C₃₋₇ cycloalkyl group;
R⁸, R⁹, and R¹⁰ are each independently selected from H and C₁₋₄ alkyl;
j is 0 or 1;
k is 0, 1, 2, 3, 4, 5, or 6;
l is 0 or 1;
m is 0, 1, 2, 3, 4, 5, or 6; and
n is 0 or 1;
wherein when j is 0, then 1 is 1,
wherein j and 1 are not both 0,
with the proviso that the compound is other than:

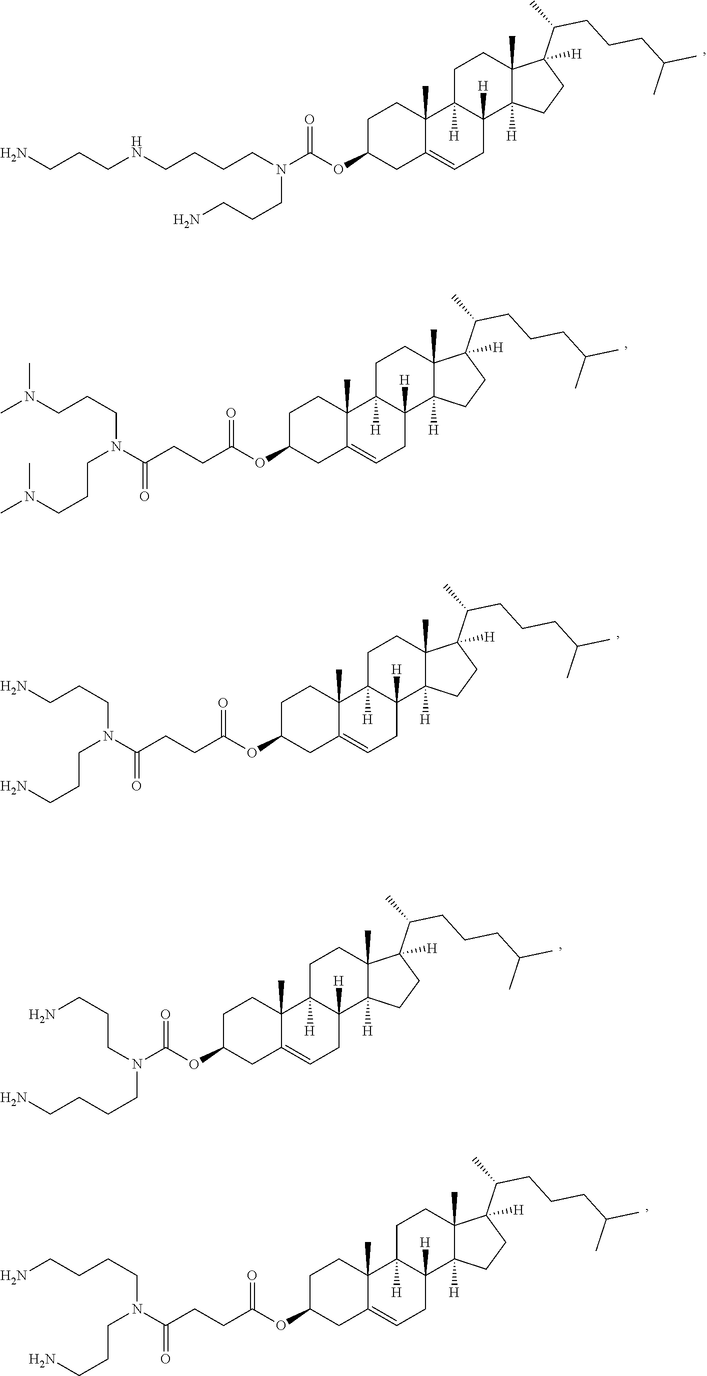

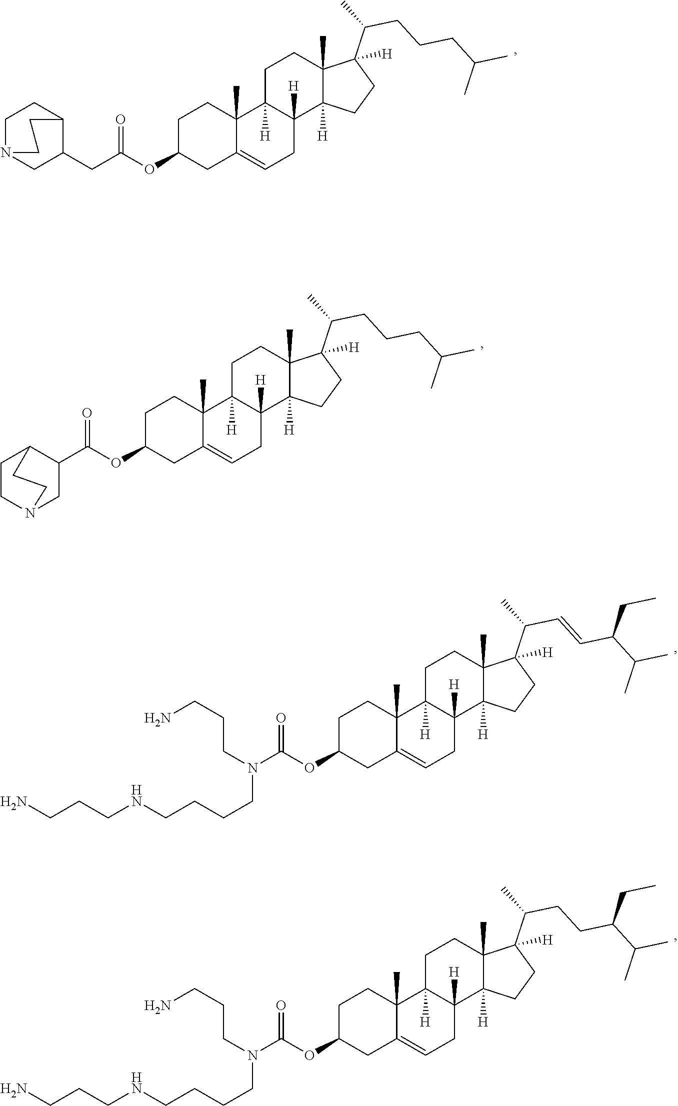

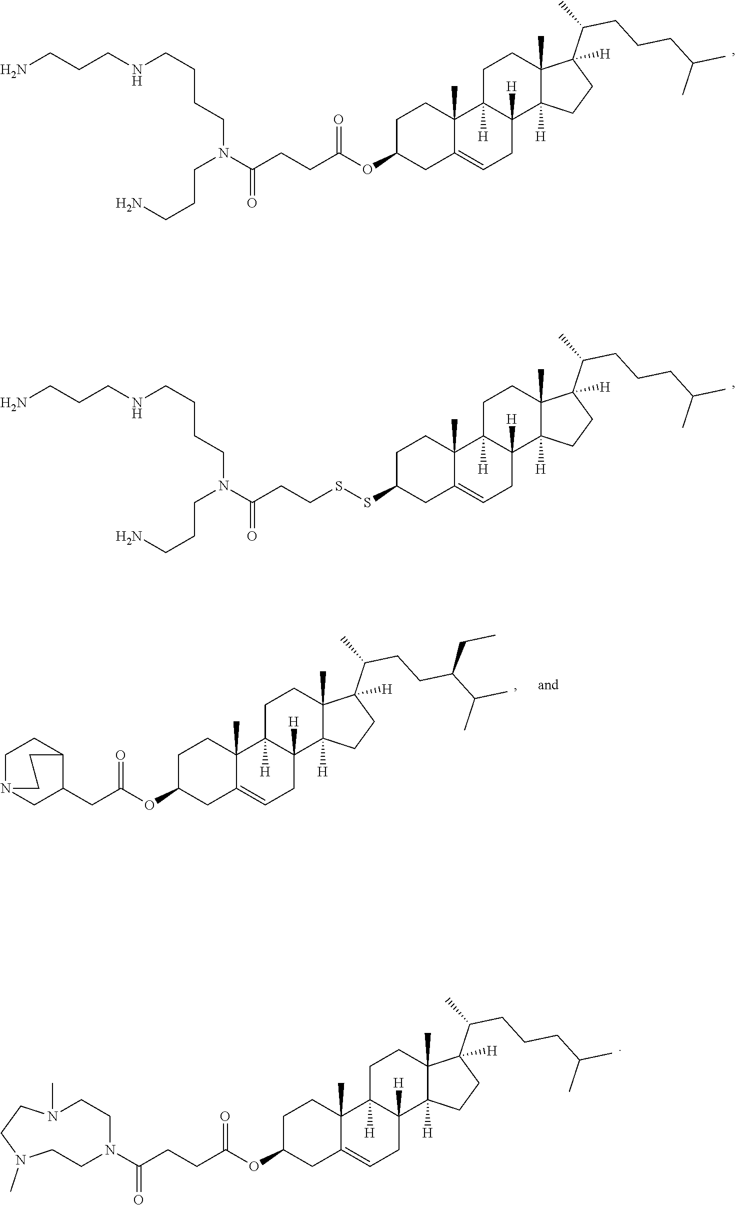

2. A lipid amine compound of Formula A1: 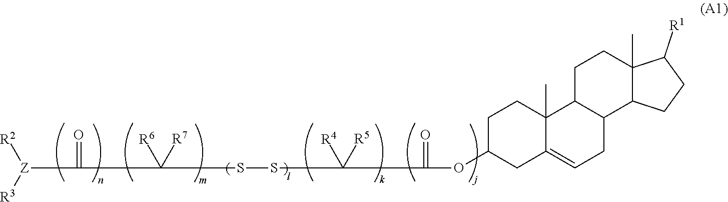
or a salt thereof, wherein:
Z is N or CH;
R¹ is C₁₋₁₄ alkyl, C₁₋₁₄ alkenyl, or C₁₋₁₄ hydroxyalkyl;
R² and R³ are each C₂₋₂₀ alkyl, wherein:
(i) the C₂₋₂₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₂₋₂₀ alkyl are optionally replaced with O; and
(iii) 1, 2, 3, or 4 non-terminal carbons of the C₂₋₂₀ alkyl are optionally replaced with NR¹⁰;
wherein R² and R³ are the same or different;
or R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from CI-4 alkyl, —NR⁸R⁹, OH, and halo;
or R², R³, and R⁶, together with the atoms to which they are attached and any intervening atoms, form a 7-18 membered bridged heterocycloalkyl group optionally substituted with 1, 2, or 3 substituents independently selected from CI-4 alkyl, —NR⁸R⁹, OH, and halo;
R⁴, R⁵, R⁶, and R⁷ are each independently selected from H, halo, and C₁₋₄ alkyl;
or R⁴ and R⁵ together with the carbon atom to which they are attached form a C₃₋₇ cycloalkyl group;
or R⁶ and R⁷ together with the carbon atom to which they are attached form a C₃₋₇ cycloalkyl group;
R⁸, R⁹, and R¹⁰ are each independently selected from H and C₁₋₄ alkyl;
j is 0 or 1;
k is 0, 1, 2, 3, 4, 5, or 6;
l is 0 or 1;
m is 0, 1, 2, 3, 4, 5, or 6; and
n is 0 or 1;
wherein when j is 0, then 1 is 1,
wherein j and 1 are not both 0,
with the proviso that the compound is other than:

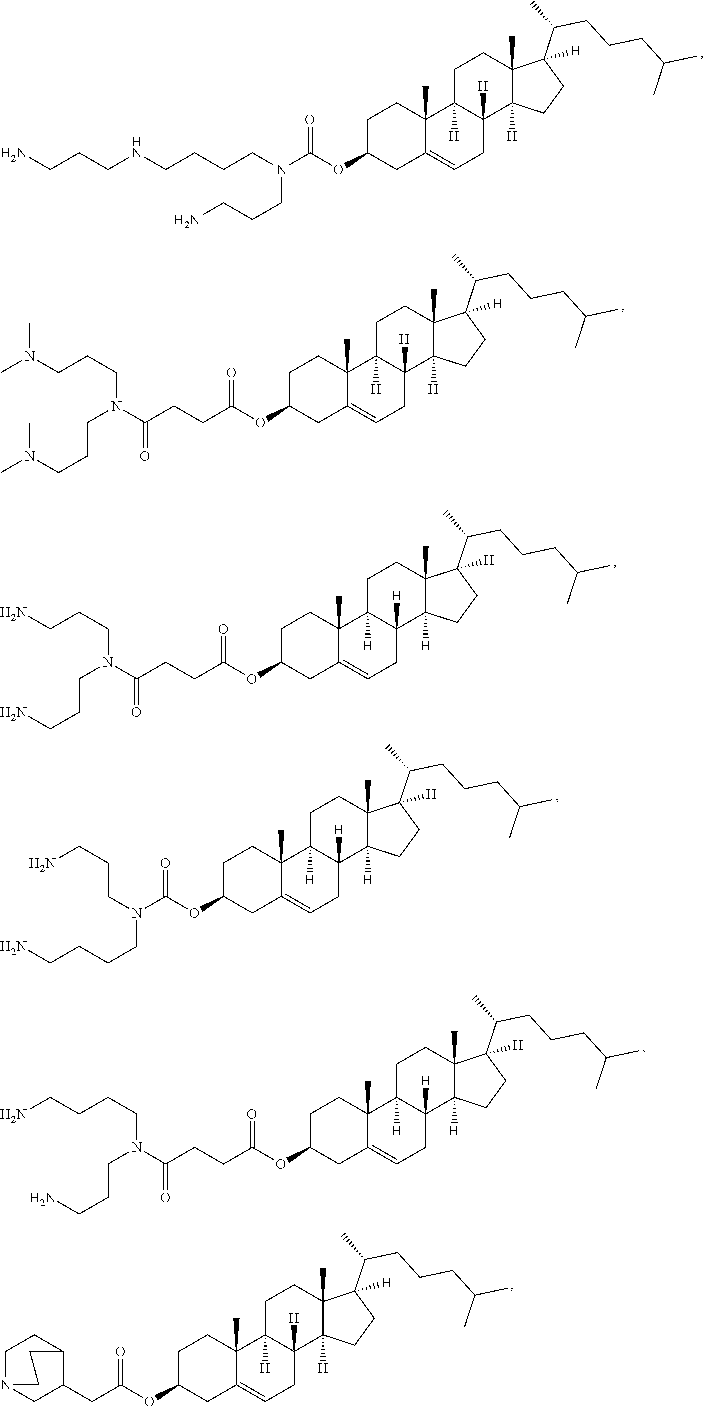

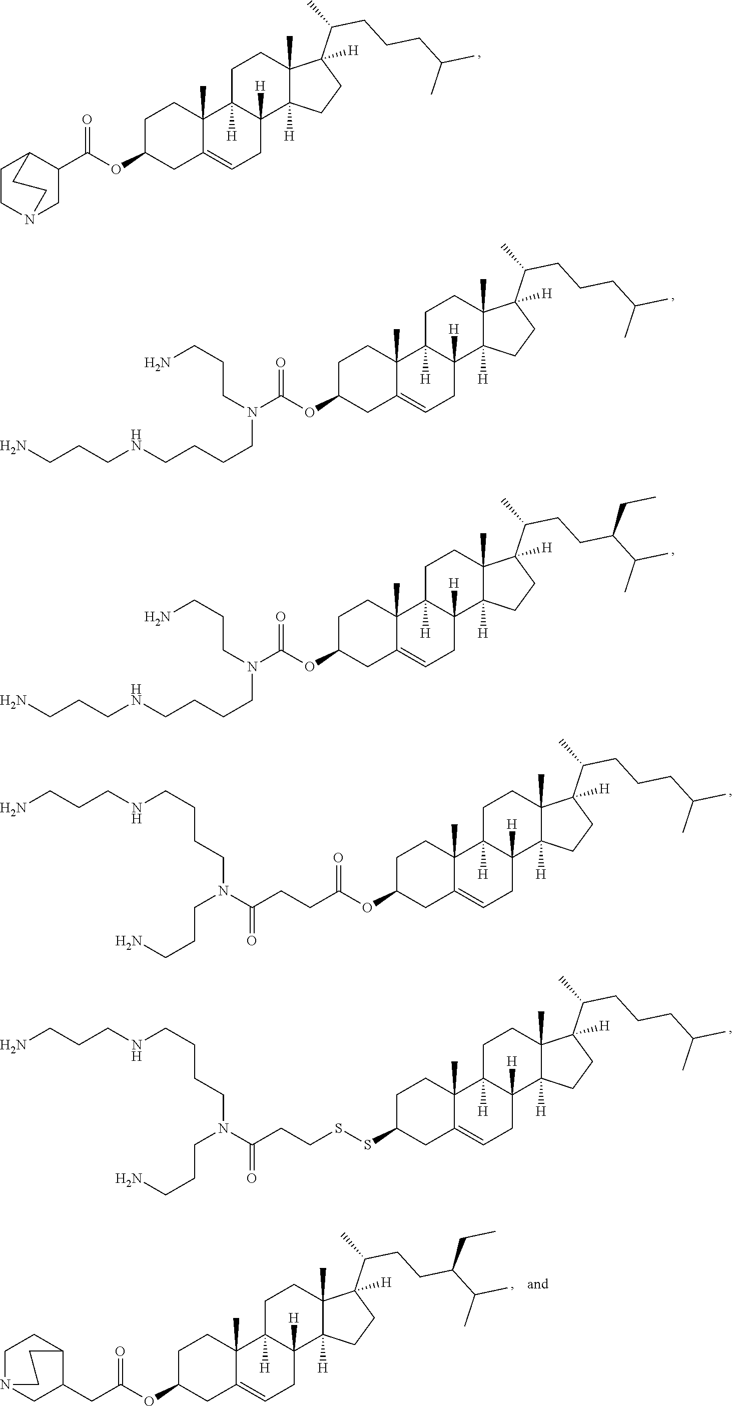

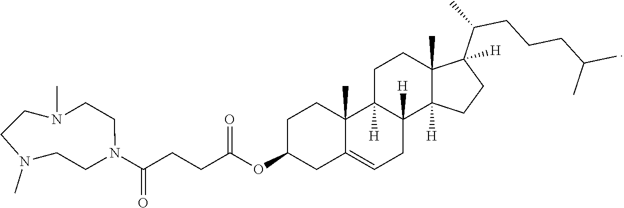

3. The compound of claims 1 or 2, or a salt thereof, wherein Z is N.

4. The compound of claims 1 or 2, or a salt thereof, wherein Z is CH.

5. The compound of any one of claims 1-4, or a salt thereof, wherein R¹ is C₁₋₁₄ alkyl.

6. The compound of any one of claims 1-4, or a salt thereof, wherein R¹ is C₁₋₁₄ hydroxyalkyl.

7. The compound of any one of claims 1-4, or a salt thereof, wherein R¹ is C₁₋₁₄ alkenyl.

8. The compound of any one of claims 1-4, wherein R¹ is

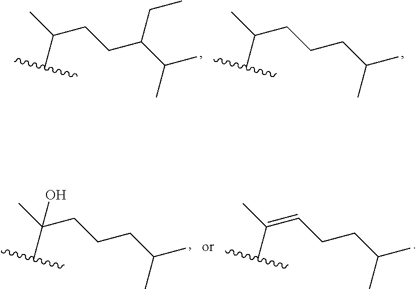

9. The compound of any one of claims 1-8, or a salt thereof, wherein j is 0.

10. The compound of any one of claims 1-8, or a salt thereof, wherein j is 1.

11. The compound of any one of claims 1-10, or a salt thereof, wherein k is 0, 2, 3, or 4.

12. The compound of any one of claims 1-10, or a salt thereof, wherein k is 0.

13. The compound of any one of claims 1-10, or a salt thereof, wherein k is 2.

14. The compound of any one of claims 1-10, or a salt thereof, wherein k is 3.

15. The compound of any one of claims 1-10, or a salt thereof, wherein k is 4.

16. The compound of any one of claims 1-15, or a salt thereof, wherein 1 is 0.

17. The compound of any one of claims 1-15, or a salt thereof, wherein 1 is 1.

18. The compound of any one of claims 1-17, or a salt thereof, wherein m is 0, 1, 2, or 4.

19. The compound of any one of claims 1-17, or a salt thereof, wherein m is 0.

20. The compound of any one of claims 1-17, or a salt thereof, wherein m is 1.

21. The compound of any one of claims 1-17, or a salt thereof, wherein m is 2.

22. The compound of any one of claims 1-17, or a salt thereof, wherein m is 4.

23. The compound of any one of claims 1-22, or a salt thereof, wherein n is 0.

24. The compound of any one of claims 1-22, or a salt thereof, wherein n is 1.

25. The compound of any one of claims 1-8, or a salt thereof, wherein j is 0, k is 0, 1 is 1, m is 1, and n is 1.

26. The compound of any one of claims 1-8, or a salt thereof, wherein j is 0, k is 0, 1 is 1, m is 2, and n is 1.

27. The compound of any one of claims 1-8, or a salt thereof, wherein j is 0, k is 0, 1 is 1, m is 4, and n is 1.

28. The compound of any one of claims 1-8, or a salt thereof, wherein j is 1, k is 0, 1 is 0, m is 0, and n is 0.

29. The compound of any one of claims 1-8, or a salt thereof, wherein j is 1, k is 1, 1 is 0, m is 0, and n is 0.

30. The compound of any one of claims 1-8, or a salt thereof, wherein j is 1, k is 1, 1 is 0, m is 0, and n is 1.

31. The compound of any one of claims 1-8, or a salt thereof, wherein j is 1, k is 1, 1 is 0, m is 2, and n is 0.

32. The compound of any one of claims 1-8, or a salt thereof, wherein j is 1, k is 1, 1 is 1, m is 1, and n is 1.

33. The compound of any one of claims 1-8, or a salt thereof, wherein j is 1, k is 2, 1 is 0, m is 0, and n is 0.

34. The compound of any one of claims 1-8, or a salt thereof, wherein j is 1, k is 2, 1 is 0, m is 0, and n is 1.

35. The compound of any one of claims 1-8, or a salt thereof, wherein j is 1, k is 3, 1 is 0, m is 0, and n is 1.

36. The compound of any one of claims 1-8, or a salt thereof, wherein j is 1, k is 4, 1 is 0, m is 0, and n is 1.

37. The compound of any one of claims 1 and 3-36, or a salt thereof, wherein:
R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₂₋₁₀ alkyl are optionally replaced with O;
(iii) 1 or 2 non-terminal carbons of the C₂₋₁₀ alkyl are optionally replaced with NR¹⁰;
(iv) 1 or 2 non-terminal carbons of the C₂₋₁₀ alkyl are optionally replaced with C(═O); and
(v) 1 or 2 non-terminal carbons of the C₂₋₁₀ alkyl are optionally replaced with CR^{a}R^{b} wherein R^{a} and R^{b} together with the C atom to which they are attached form a C₃₋₆ cycloalkyl group.

38. The compound of any one of claims 1-36, or a salt thereof, wherein:
R² and R³ are each independently selected from C₂₋₁₀ alkyl, wherein:
(i) the C₂₋₁₀ alkyl is substituted by 1, 2, 3, or 4 substituents independently selected from —NR⁸R⁹, OH, and halo, wherein at least one substituent is —NR⁸R⁹;
(ii) 1 or 2 non-terminal carbons of the C₂₋₁₀ alkyl are optionally replaced with O; and
(iii) 1 or 2 non-terminal carbons of the C₂₋₁₀ alkyl are optionally replaced with NR¹⁰.

39. The compound of any one of claims 1 and 3-36, or salt thereof, wherein one of R² and R³ is C₂₋₅ alkyl, wherein: the C₂₋₅ alkyl is substituted by 1, 2, 3, 4, or 5 substituents independently selected from —NR⁸R⁹, OH, and halo, wherein at least one substituent is —NR⁸R⁹;
and wherein the other of R² and R³ is C₇₋₁₀ alkyl, wherein:
(i) the C₇₋₁₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₇₋₁₀ alkyl are optionally replaced with O;
(iii) 1, 2, 3, or 4 non-terminal carbons of the C₇₋₁₀ alkyl are optionally replaced with NR¹⁰;
(iv) 1, 2, 3, or 4 non-terminal carbons of the C₇₋₁₀ alkyl are optionally replaced with C(═O); and
(v) 1, 2, 3, or 4 non-terminal carbons of the C₇₋₁₀ alkyl are optionally replaced with CR^{a}R^{b} wherein R^{a} and R^{b} together with the C atom to which they are attached form a C₃₋₆ cycloalkyl group.

40. The compound of any one of claims 1-36, or a salt thereof, wherein one of R² and R³ is C₂₋₅ alkyl, wherein:
(i) the C₂₋₅ alkyl is substituted by 1, 2, 3, 4, or 5 substituents independently selected from —NR⁸R⁹, OH, and halo, wherein at least one substituent is —NR⁸R⁹;
and wherein the other of R² and R³ is C₇₋₁₀ alkyl, wherein:
(i) the C₇₋₁₀ alkyl is substituted by 1, 2, 3, 4, or 5 substituents independently selected from —NR⁸R⁹, OH, and halo, wherein at least one substituent is —NR⁸R⁹;
(ii) 1, 2, 3, or 4 non-terminal carbons of the C₇₋₁₀ alkyl are optionally replaced with O; and
(iii) 1, 2, 3, or 4 non-terminal carbons of the C₇₋₁₀ alkyl are optionally replaced with NR¹⁰.

41. The compound of any one of claims 1-36, or a salt thereof, wherein one of R² and R³ is C₃ alkyl which is substituted by at least one —NR⁸R⁹ group and is further optionally substituted by one or two groups selected from OH and halo.

42. The compound of any one of claims 1-36, or a salt thereof, wherein:
R² and R³ together with the N atom to which they are attached form a 7-18 membered heterocycloalkyl group comprising 1, 2, or 3 ring-forming NR¹⁰ groups, wherein the 7-18 membered heterocycloalkyl group is optionally substituted with 1, 2, or 3 substituents independently selected from C₁₋₄ alkyl, —NR⁸R⁹, OH, and halo.

43. The compound of any one of claims 1-36, or a salt thereof, wherein:
R² and R³ together with the N atom to which they are attached form a 8-10 membered heterocycloalkyl group comprising 1, 2, or 3 ring-forming NR¹⁰ groups, wherein the 8-10 membered heterocycloalkyl group is optionally substituted with 1, 2, or 3 substituents independently selected from C₁₋₄ alkyl, —NR⁸R⁹, OH, and halo.

44. The compound of any one of claims 1-36, or a salt thereof, wherein:
R² and R³ together with the N atom to which they are attached form a heterocycloalkyl group of formula:

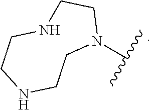 [SMILES: *N1CCNCCNCC1]

45. The compound of any one of claims 1-11, 13-24, and 29-36, or a salt thereof, wherein:
R², R³, and R⁶, together with the atoms to which they are attached and any intervening atoms, form a 7-18 membered bridged heterocycloalkyl group optionally substituted with 1, 2, or 3 substituents independently selected from C₁₋₄ alkyl, —NR⁸R⁹, OH, and halo.

46. The compound of any one of claims 1-11, 13-24, and 29-36, or a salt thereof, wherein:
R², R³, and R⁶, together with the atoms to which they are attached and any intervening atoms, form a 7-18 membered bridged heterocycloalkyl group selected from:

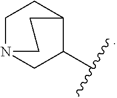 [SMILES: *C1CN2CCC1CC2]

47. The compound of any one of claims 1-11, 13-24, and 29-46, or a salt thereof, wherein R⁴ and R⁵ are each independently H or C₁₋₄ alkyl.

48. The compound of any one of claims 1-11, 13-24, and 29-46, or a salt thereof, wherein R⁴ and R⁵ are each independently H or methyl.

49. The compound of any one of claims 1-11, 13-24, and 29-46, or a salt thereof, wherein both R⁴ and R⁵ are H.

50. The compound of any one of claims 1-11, 13-24, and 29-46, or a salt thereof, wherein both R⁴ and R⁵ are C₁₋₄ alkyl.

51. The compound of any one of claims 1-11, 13-24, and 29-46, or a salt thereof, wherein one of R⁴ and R⁵ is H and the other is C₁₋₄ alkyl.

52. The compound of any one of claims 1-18, 20-27, 31, 32, and 37-51, or a salt thereof, wherein R⁶ and R⁷ are each independently H or C₁₋₄ alkyl.

53. The compound of any one of claims 1-18, 20-27, 31, 32, and 37-51, or a salt thereof, wherein R⁶ and R⁷ are each independently H or methyl.

54. The compound of any one of claims 1-18, 20-27, 31, 32, and 37-51, or a salt thereof, wherein both R⁶ and R⁷ are H.

55. The compound of any one of claims 1-18, 20-27, 31, 32, and 37-51, or a salt thereof, wherein both R⁶ and R⁷ are C₁₋₄ alkyl.

56. The compound of any one of claims 1-18, 20-27, 31, 32, and 37-51, or a salt thereof, wherein one of R⁶ and R⁷ is H and the other is C₁₋₄ alkyl.

57. The compound of any one of claims 1-43, 45, and 47-56, or a salt thereof, wherein R⁸, R⁹, and R¹⁰ are each independently selected from H and methyl.

58. The compound of any one of claims 1-43, 45, and 47-56, or a salt thereof, wherein R⁸ and R⁹ are both H.

59. The compound of any one of claims 1-43, 45, and 47-56, or a salt thereof, wherein R⁸ and R⁹ are both C₁₋₄ alkyl.

60. The compound of any one of claims 1-43, 45, and 47-56, or a salt thereof, wherein R⁸ and R⁹ are both methyl.

61. The compound of any one of claims 1-43, 45, and 47-56, or salt thereof, wherein one of R⁸ and R⁹ is H and the other is C₁₋₄ alkyl.

62. The compound of any one of claims 1-43 and 47-56, or a salt thereof, wherein R¹⁰ is H.

63. The compound of any one of claims 1-62 having Formula A2: 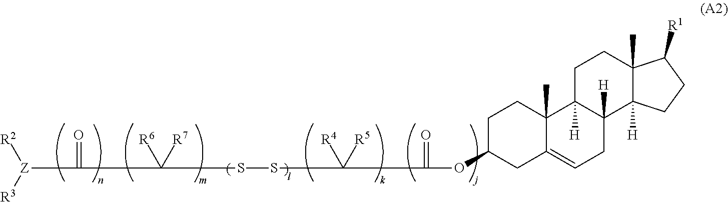
or a salt thereof.

64. The compound of claim 1 selected from:

| Lipid | |
|---|---|
| amine | |
| no. | Structure |
| SA50 | 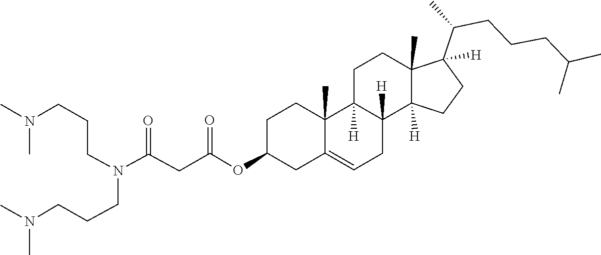 |
| SA51 | 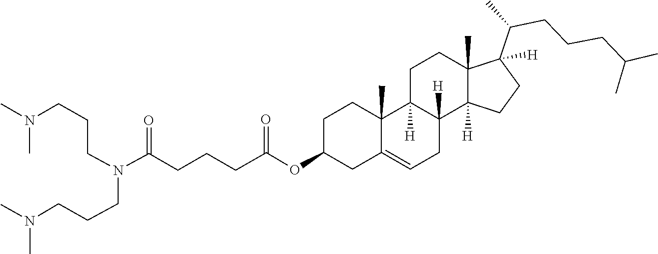 |
| SA56 | 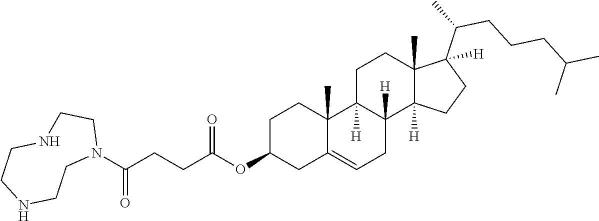 |
| SA57 | 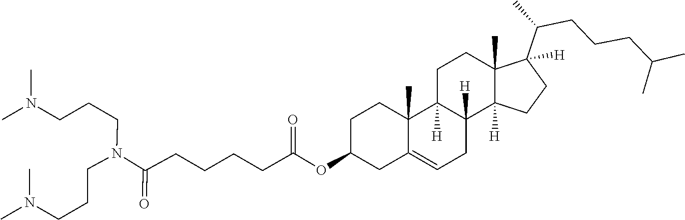 |
| SA58 | 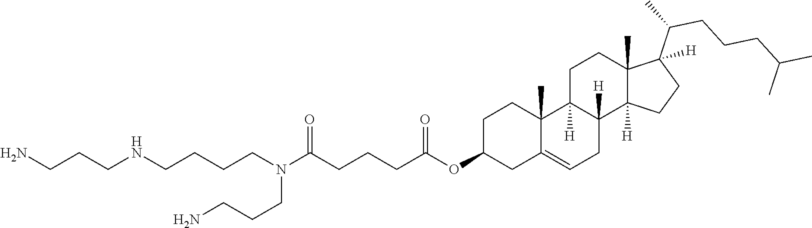 |
| SA59 | 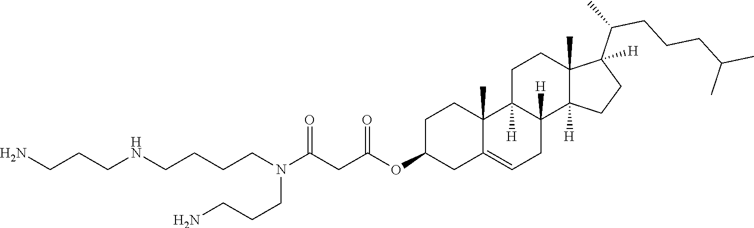 |
| SA60 | 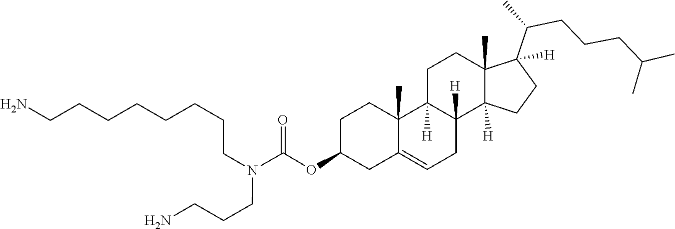 |
| SA61 | 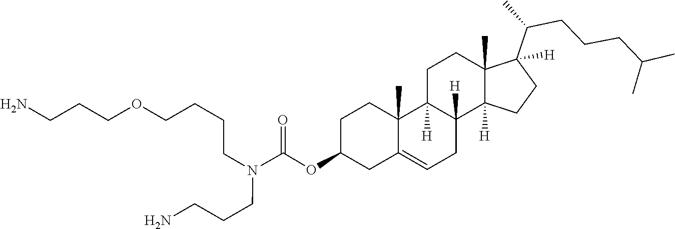 |
| SA62 | 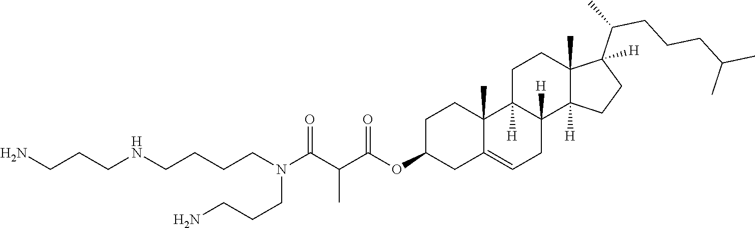 |
| SA63 | 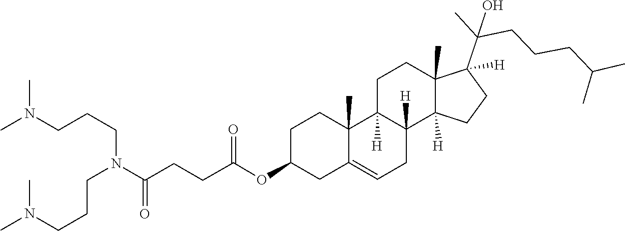 |
| SA64 | 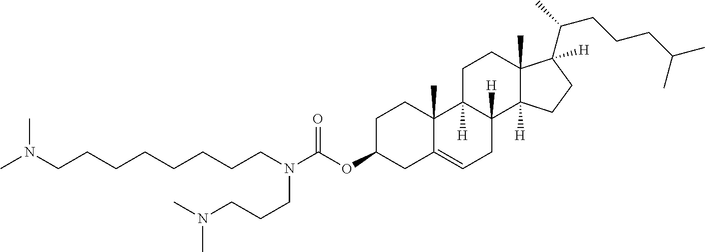 |
| SA65 | 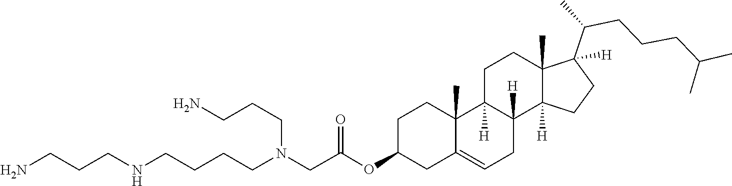 |
| SA66 | 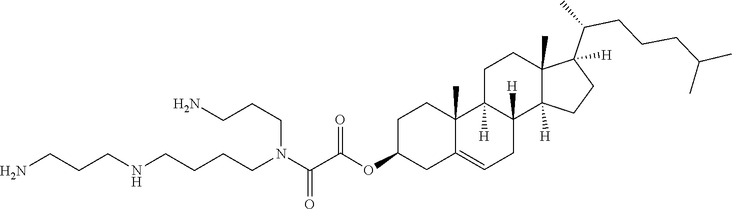 |
| SA67 | 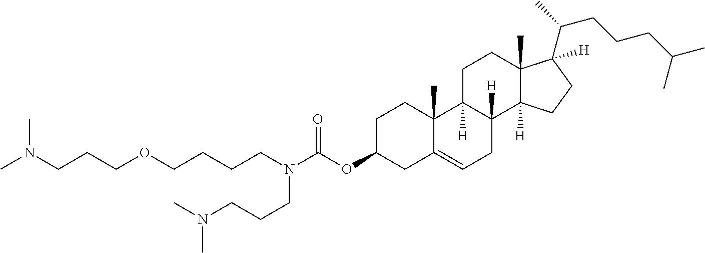 |
| SA68 | 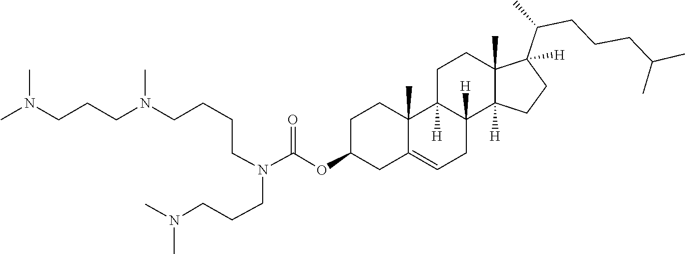 |
| SA69 | 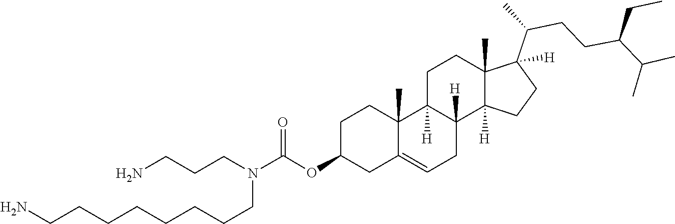 |
| SA70 | 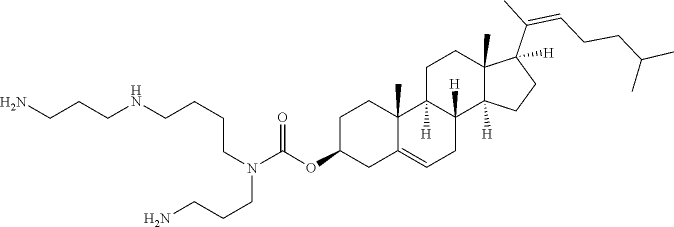 |
| SA71 | 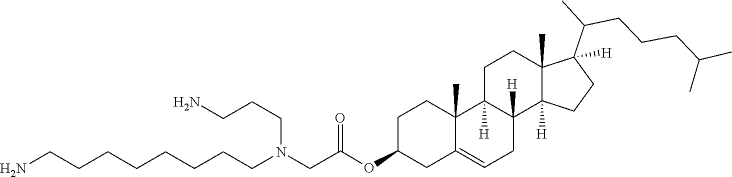 |
| SA72 | 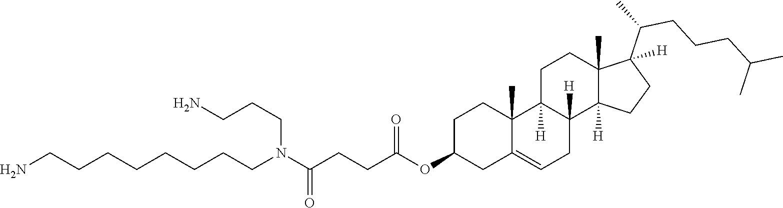 |
| SA73 | 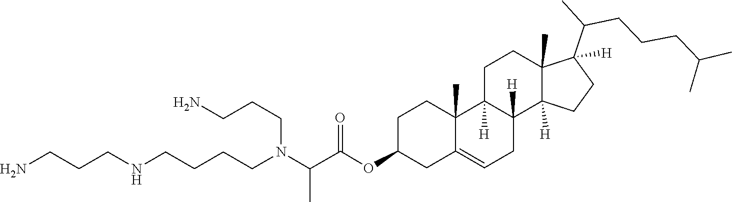 |
| SA74 | 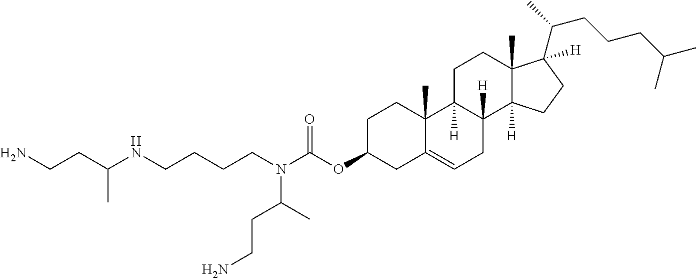 |
| SA75 | 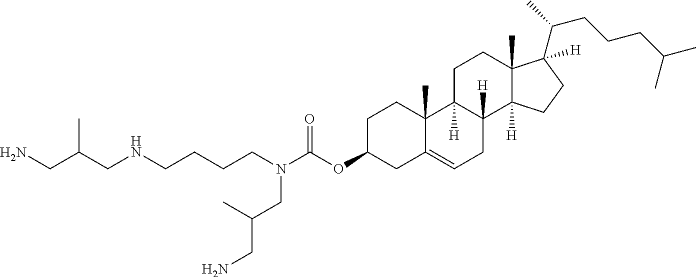 |
| SA76 | 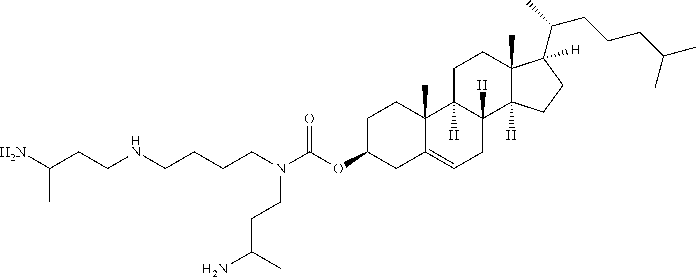 |
| SA77 | 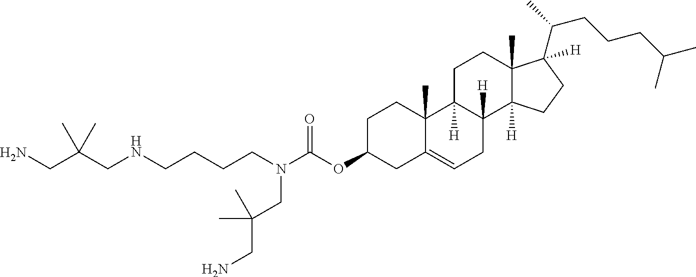 |
| SA78 | 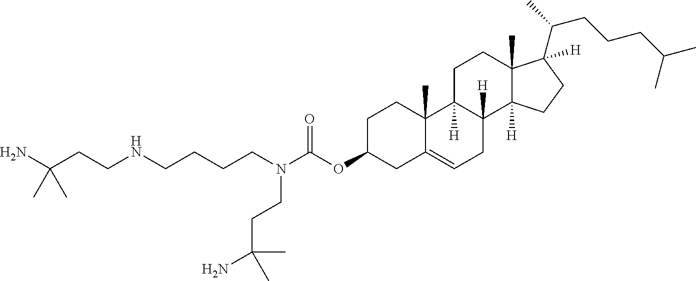 |
| SA79 | 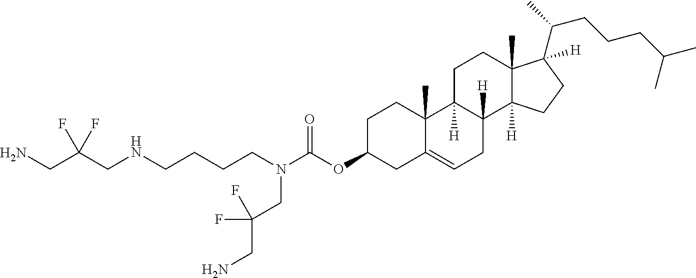 |
| SA81 | 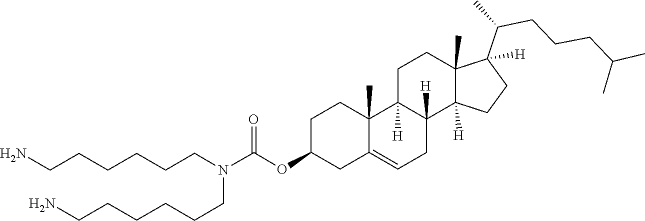 |
| SA82 | 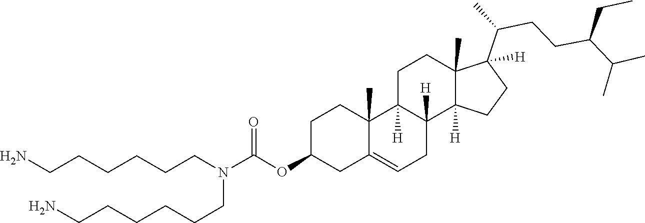 |
| SA83 | 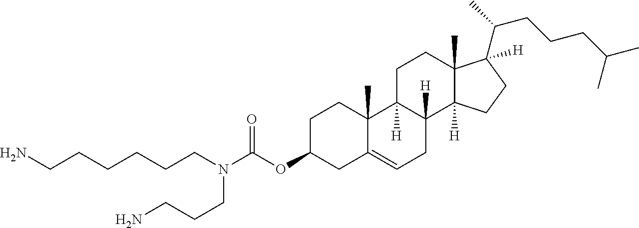 |
| SA84 | 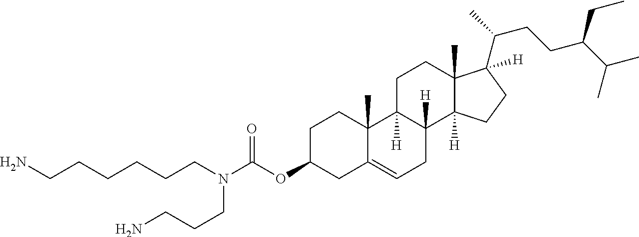 |
| SA87 | 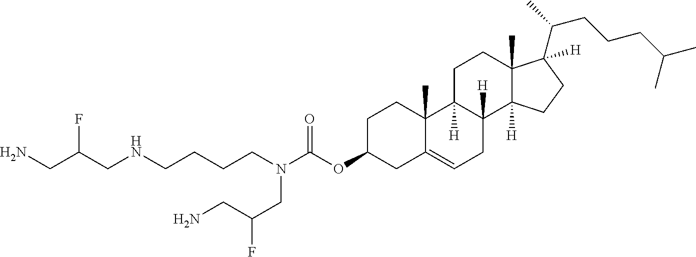 |
| SA88 | 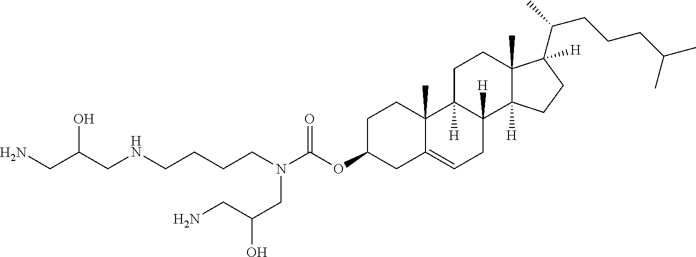 |
| SA89 | 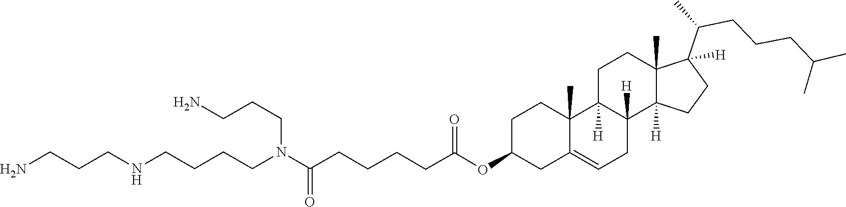 |
| SA90 | 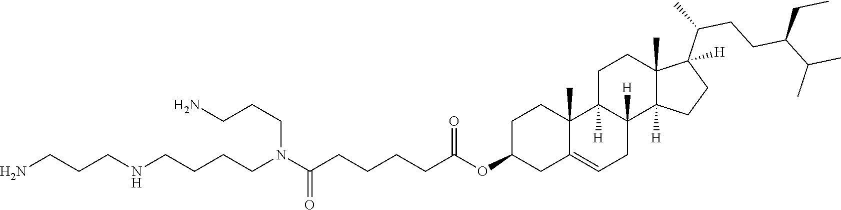 |
| SA95 | 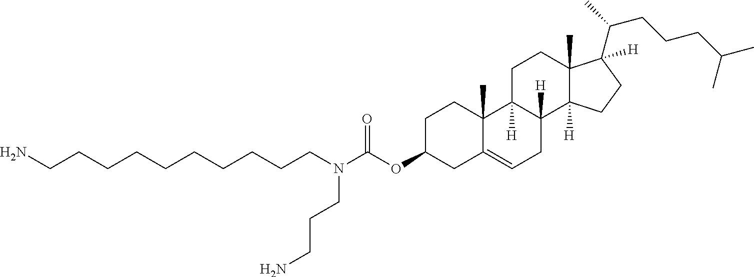 |
| SA96 | 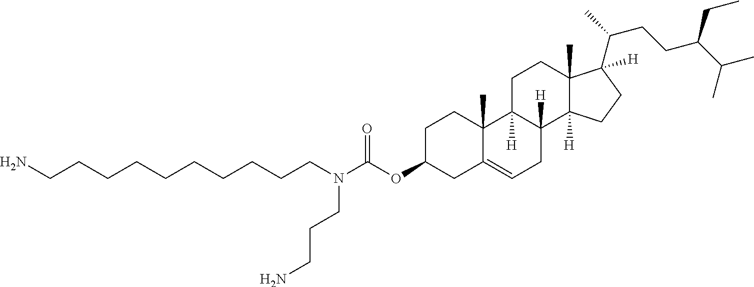 |
| SA97 | 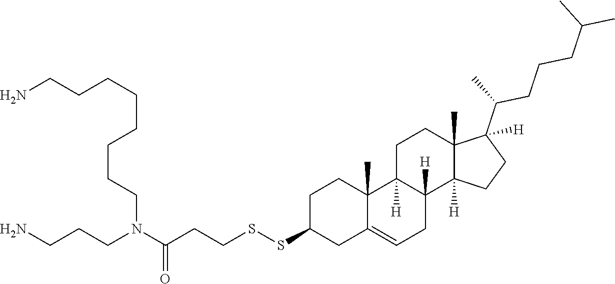 |
| SA98 | 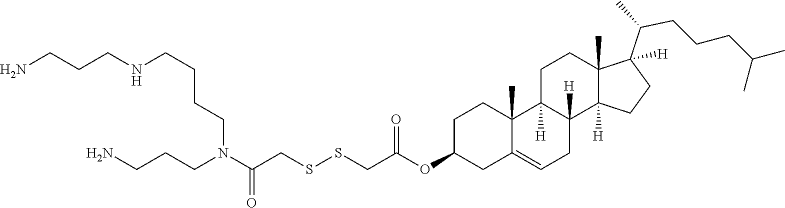 |
| SA110 | 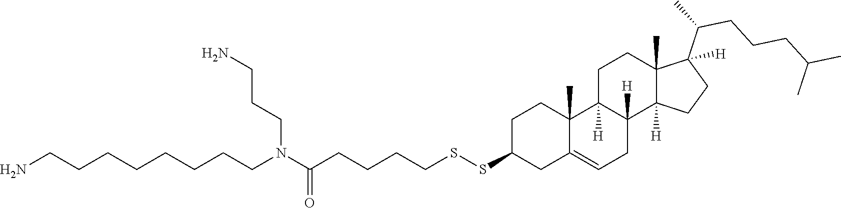 |
| SA111 | 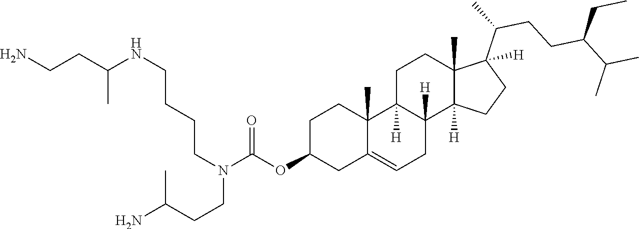 |
| SA113 | 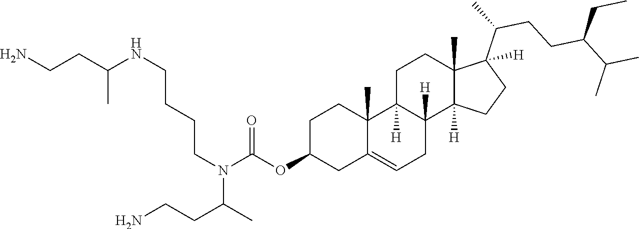 |
| SA114 | 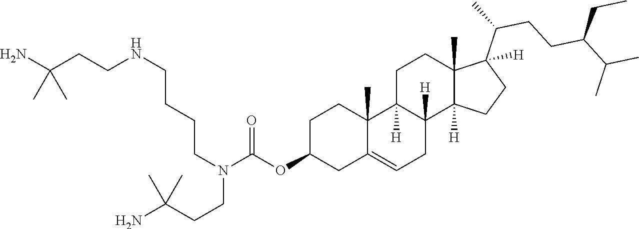 |
| SA116 | 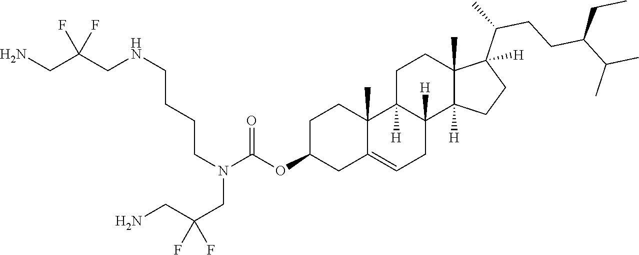 |
| SA117 | 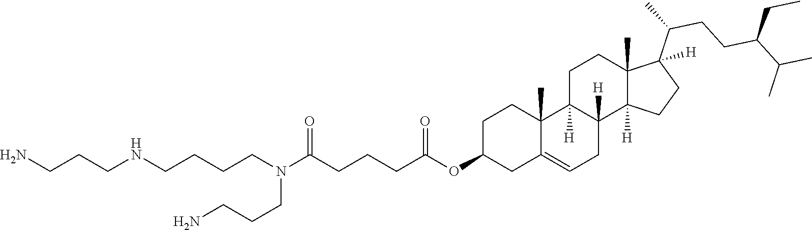 |
| SA119 | 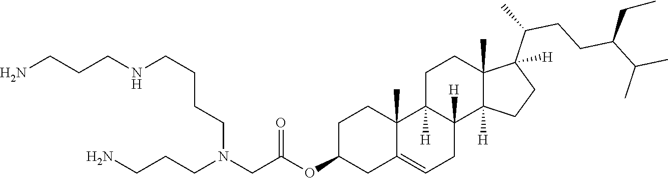 |
| SA120 | 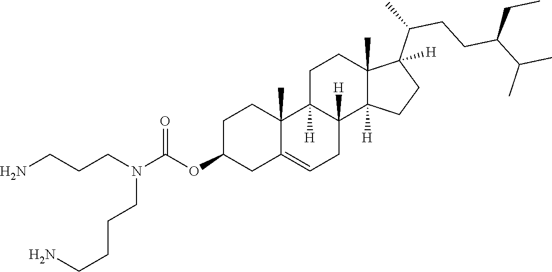 |

or a salt of any of the aforementioned.

65. The compound of claim 1 selected from:

| No. | Structure |
|---|---|
| SA118 | 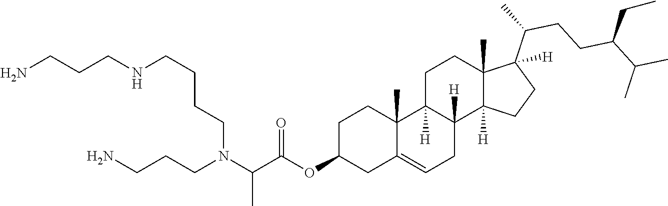 |
| SA121 | 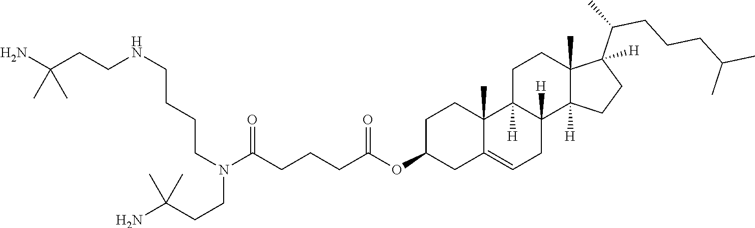 |
| SA122 | 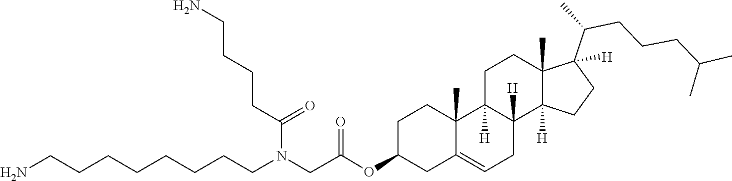 |
| SA123 | 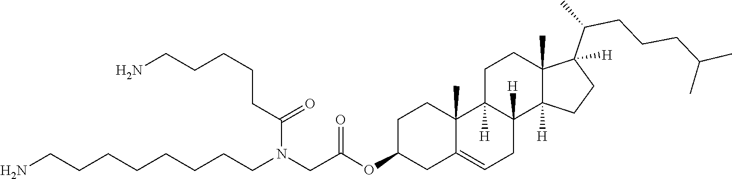 |
| SA124 | 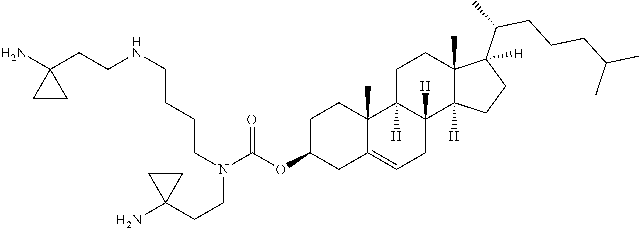 |
| SA125 | 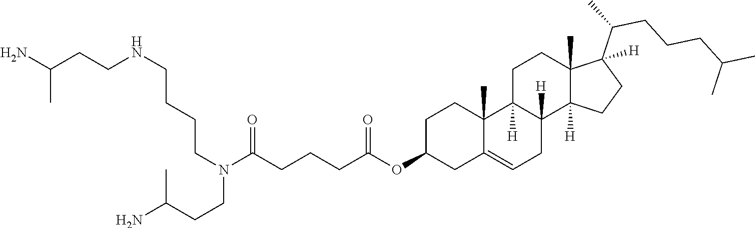 |
| SA126 | 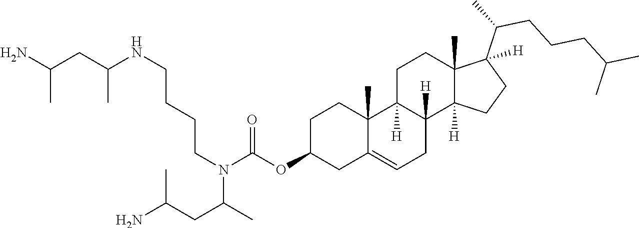 |
| SA127 | 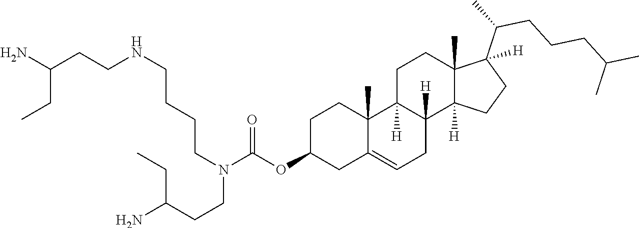 |
| SA128 | 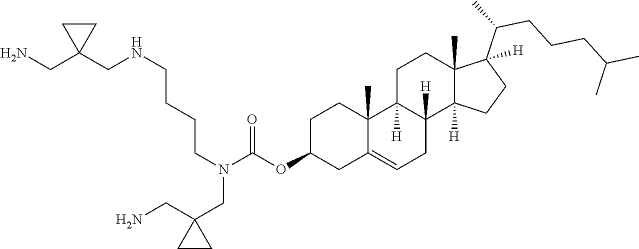 |
| SA129 | 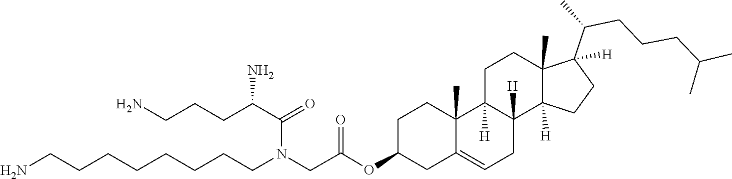 |
| SA130 | 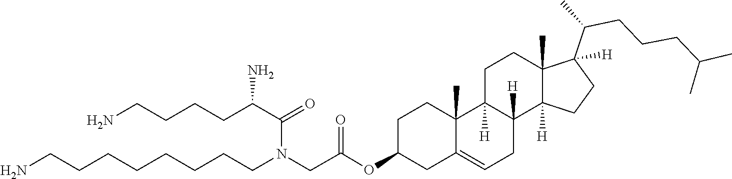 |
| SA131 | 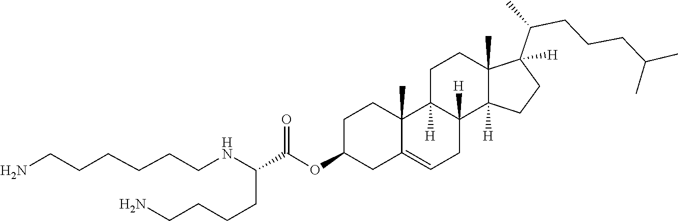 |
| SA132 | 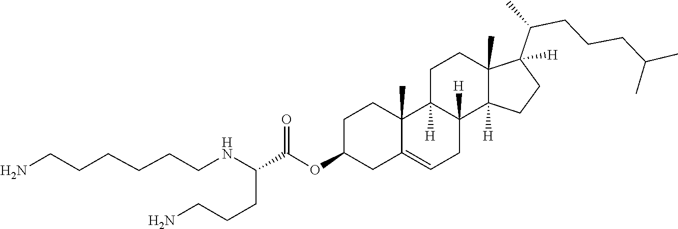 |
| SA133 | 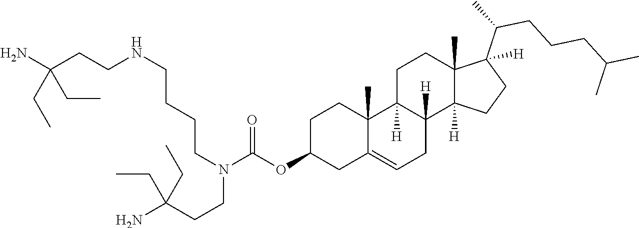 |
| SA134 | 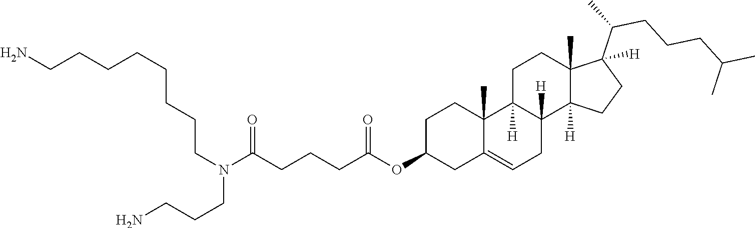 |
| SA135 | 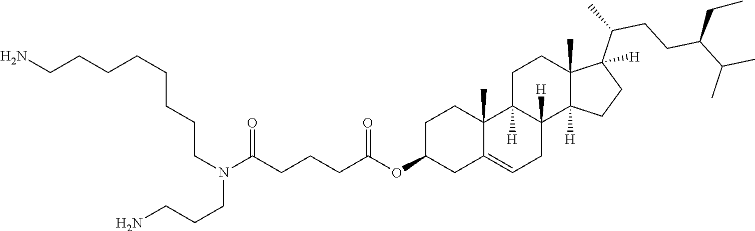 |
| SA136 | 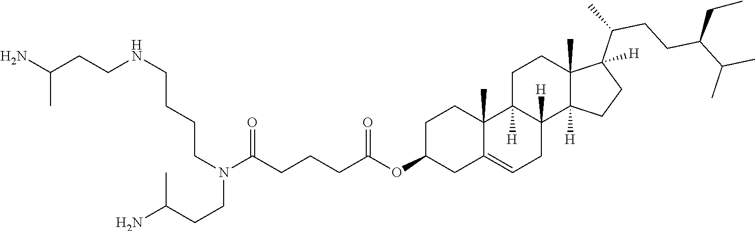 |
| SA137 | 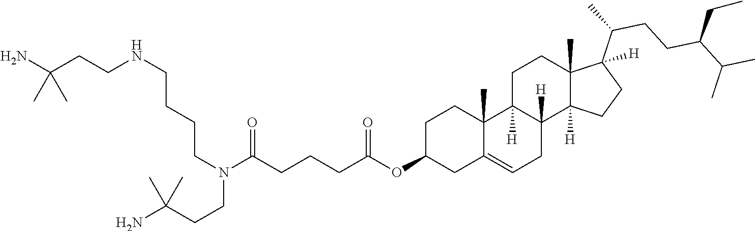 |
| SA138 | 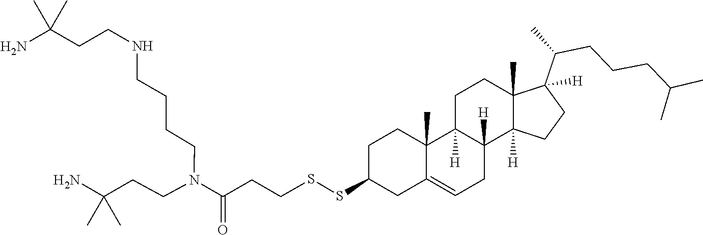 |

or a salt of any of the aforementioned.

66. The compound of claim 1 selected from:

| No. | Structure |
|---|---|
| SA139 | 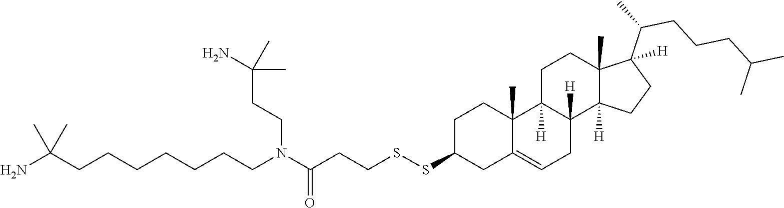 |
| SA141 | 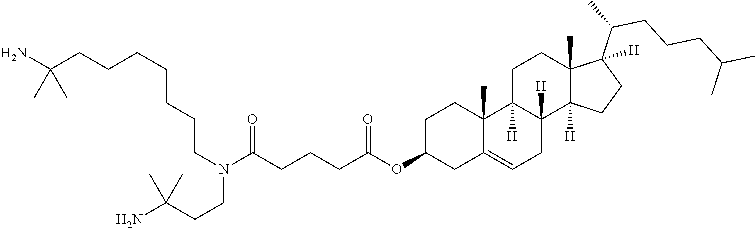 |
| SA142 | 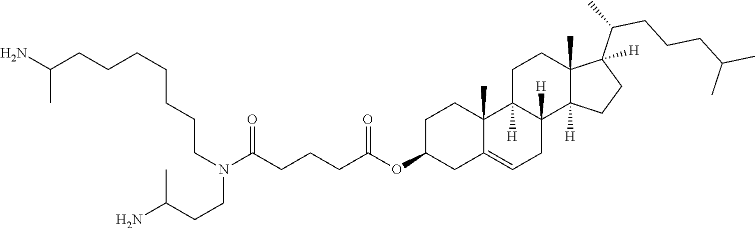 |
| SA144 | 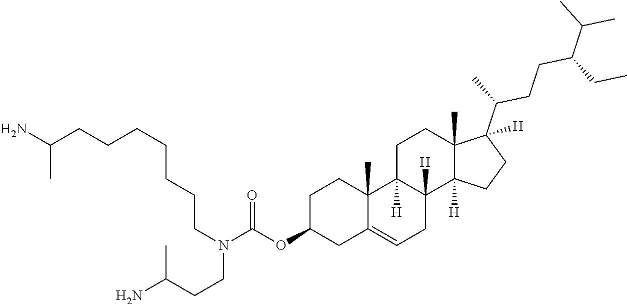 |
| SA145 | 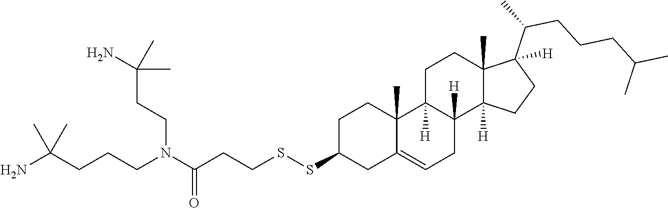 |
| SA149 | 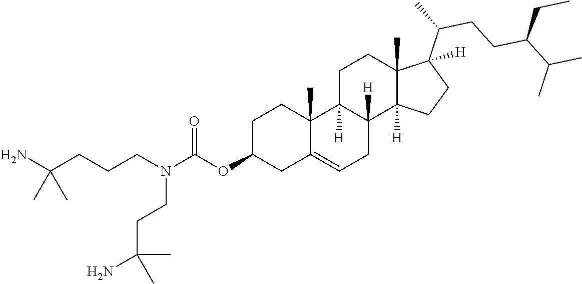 |
| SA151 | 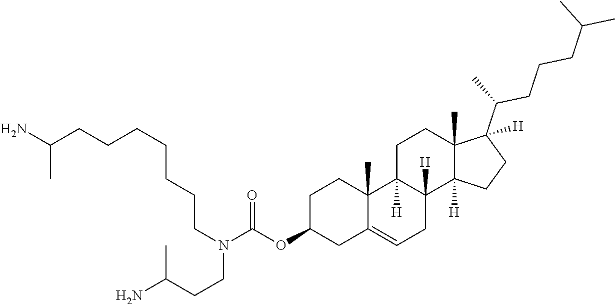 |
| SA152 | 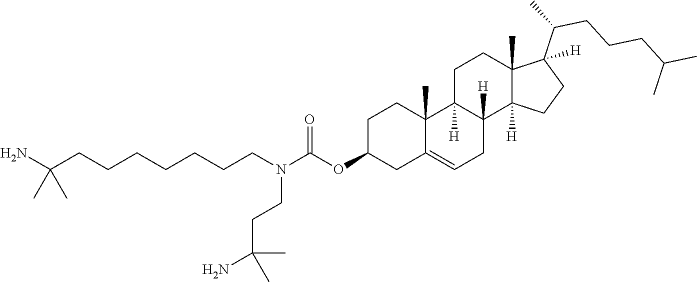 |
| SA153 | 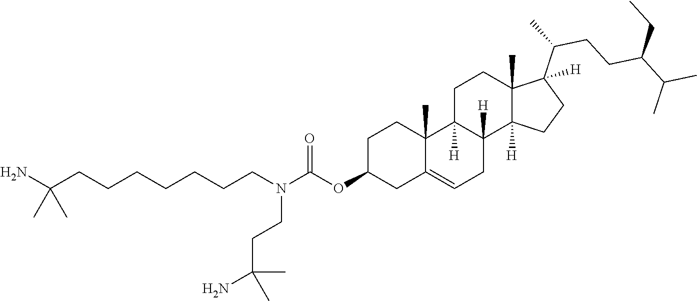 |
| SA154 | 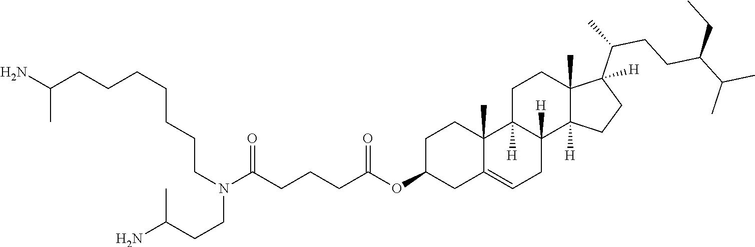 |
| SA155 | 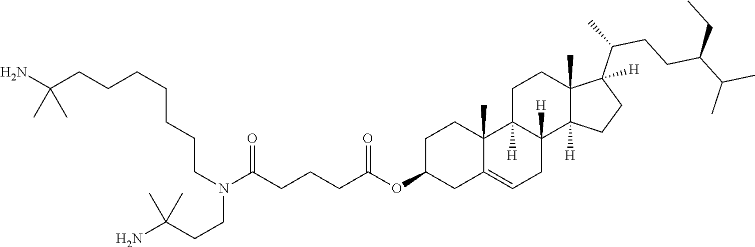 |
| SA156 | 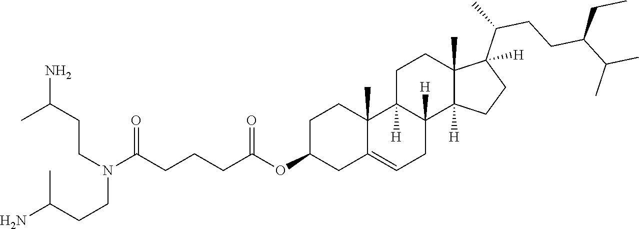 |
| SA157 | 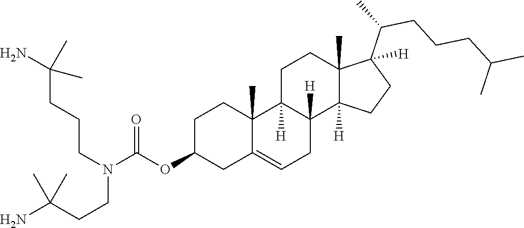 |
| SA158 | 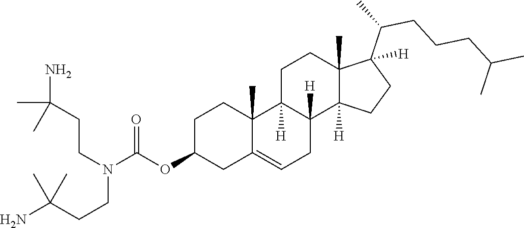 |
| SA159 | 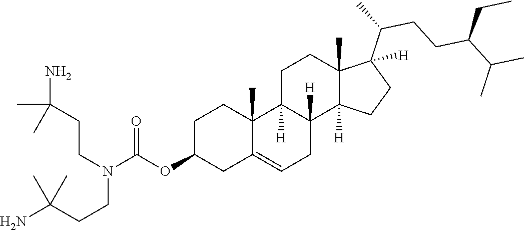 |
| SA160 | 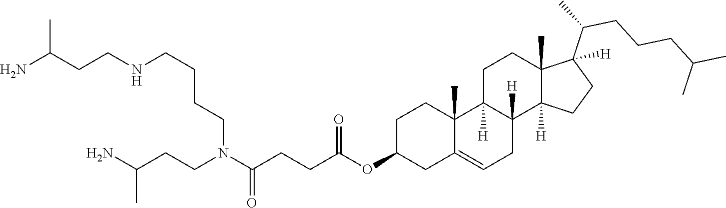 |
| SA161 | 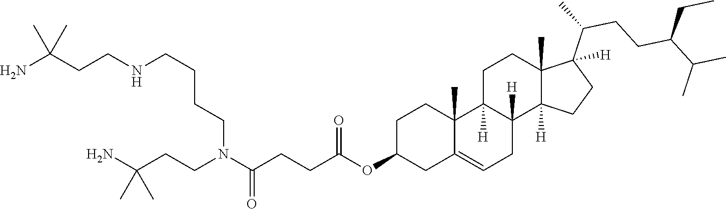 |
| SA162 | 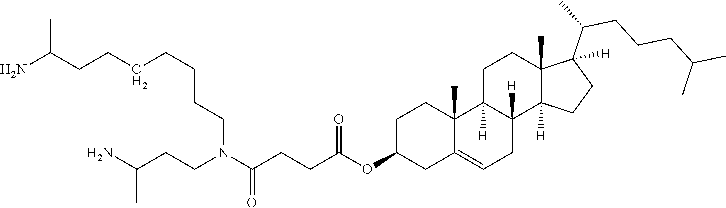 |
| SA163 | 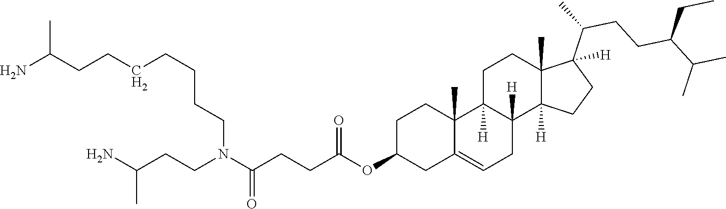 |
| SA164 | 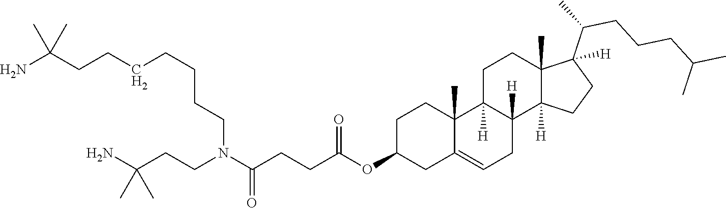 |
| SA165 | 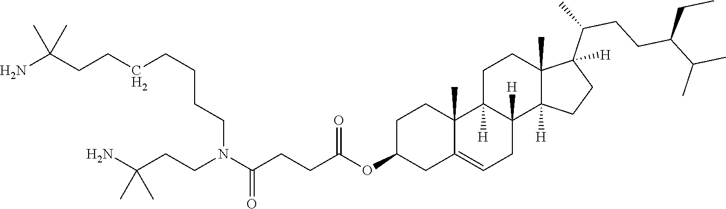 |
| SA166 | 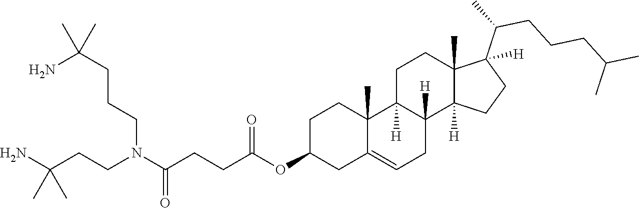 |
| SA167 | 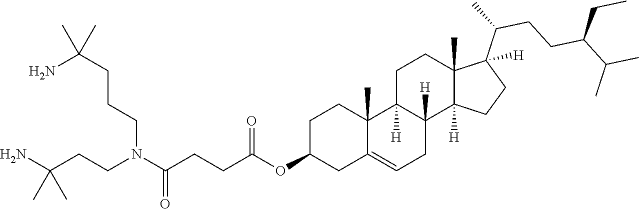 |
| SA168 | 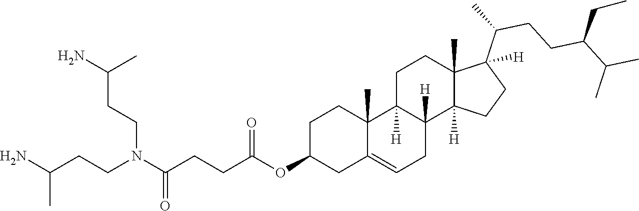 |
| SA169 | 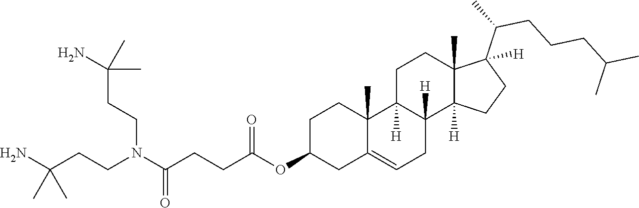 |
| SA170 | 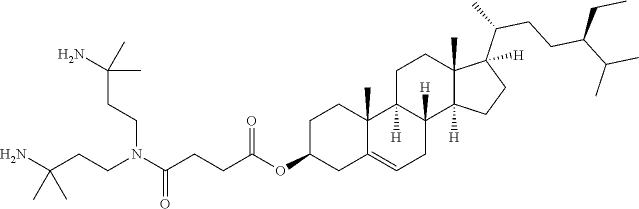 |
| SA171 | 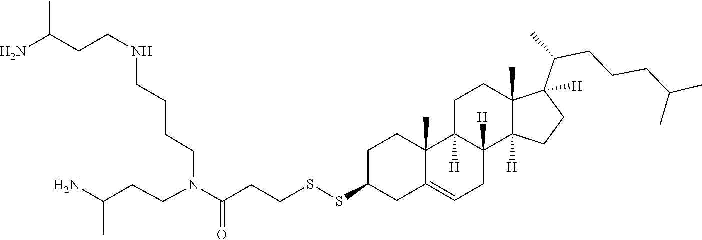 |
| SA172 | 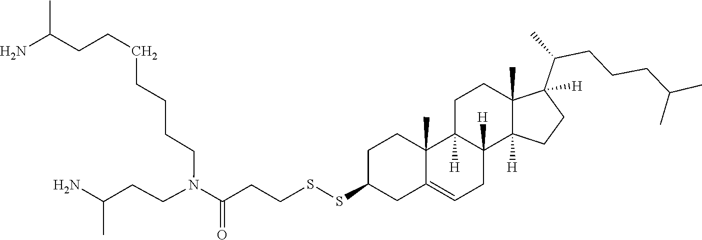 |
| SA173 | 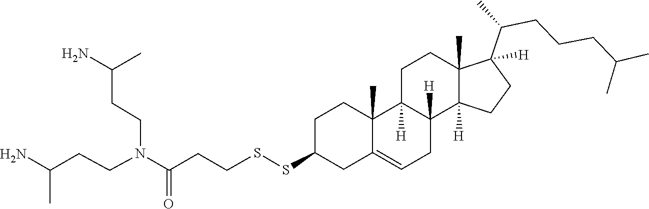 |
| SA174 | 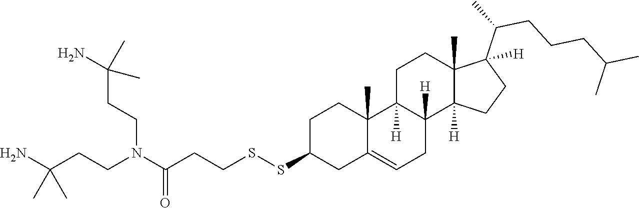 |
| SA175 | 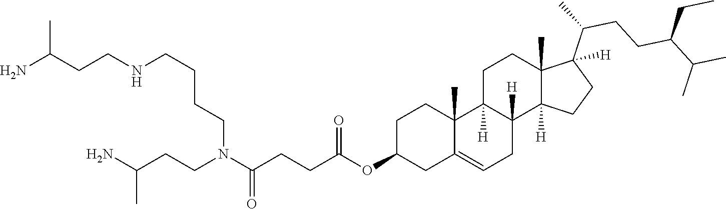 |
| SA176 | 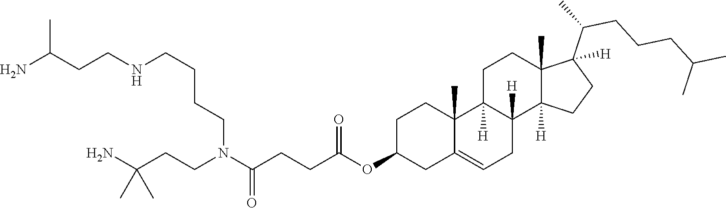 |
| SA177 | 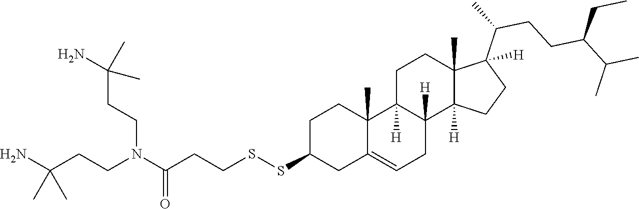 |
| SA178 | 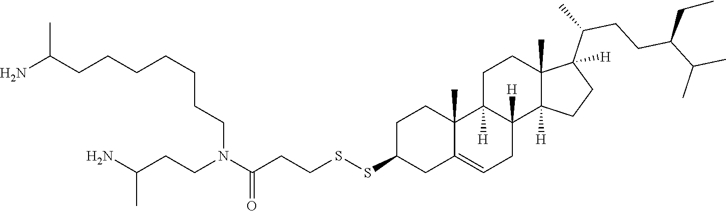 |
| SA179 | 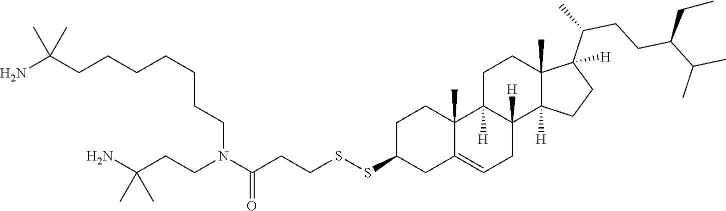 |
| SA180 | 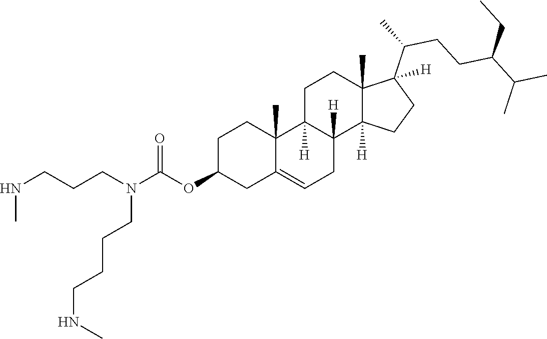 |
| SA181 | 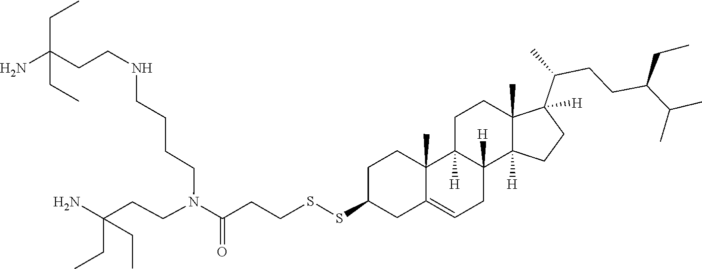 |
| SA182 | 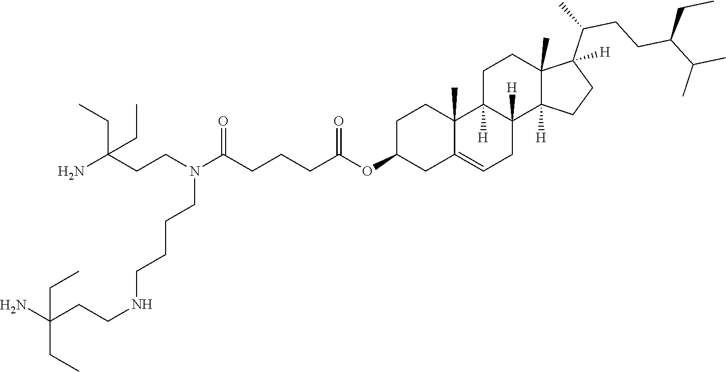 |
| SA183 | 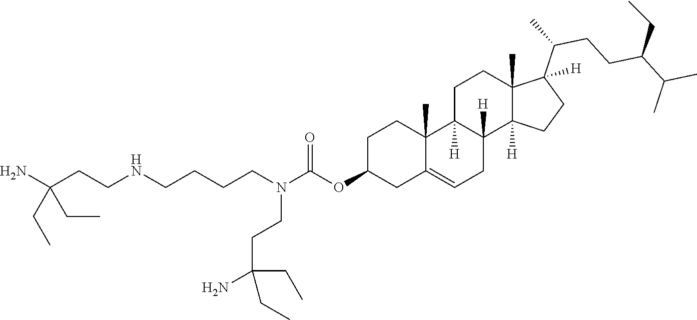 |
| SA184 | 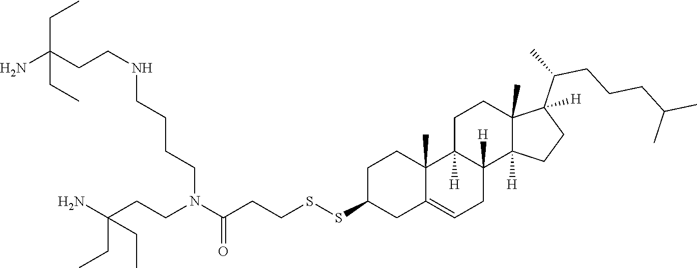 |
| SA185 | 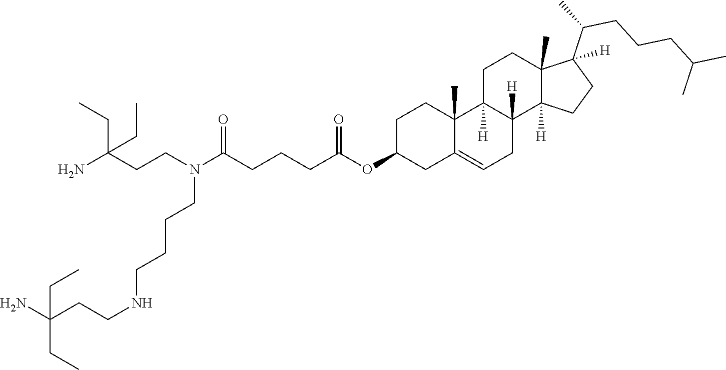 |

or a salt of any of the aforementioned.

67. A lipid nanoparticle composition comprising a lipid amine compound according to any one of claims 1-66, or a salt thereof.

68. The lipid nanoparticle composition of claim 67, wherein the lipid nanoparticle composition further comprises:
(i) an ionizable lipid,
(ii) a phospholipid,
(iii) a structural lipid, and
(iv) optionally a PEG-lipid, and
(v) optionally a payload for delivery into a cell.

69. The lipid nanoparticle composition of claim 68, wherein a weight ratio of the lipid amine to payload is about 1:1 to about 4:1.

70. The lipid nanoparticle composition of claim 68, wherein the weight ratio of the lipid amine to payload is about 1.25:1, about 2.5:1, or about 3.75:1.

71. The lipid nanoparticle composition of any one of claims 68-70, wherein the ionizable lipid is a compound of Formula (I): 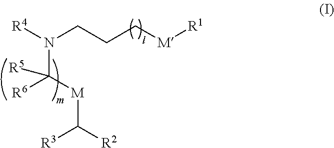
or an N-oxide or a salt thereof, wherein:
R¹ is 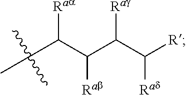
wherein 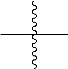
denotes a point of attachment;
R^{aα}, R^{aβ}, R^{aγ}, and R^{aδ} are each independently selected from H, C₂₋₁₂ alkyl, and C₂₋₁₂ alkenyl;
R² and R³ are each independently selected from C₁₋₁₄ alkyl and C₂₋₁₄ alkenyl;
R⁴ is selected from —(CH₂)ₙOH and 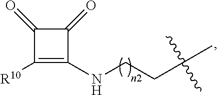
wherein n is selected from 1, 2, 3, 4, and 5;
wherein 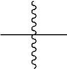
denotes a point of attachment,
wherein R¹⁰ is N(R)₂;
wherein each R is independently selected from C₁₋₆ alkyl, C₂₋₃ alkenyl, and H;
wherein n2 is selected from 1, 2, 3, 4, 5, 6, 7, 8, 9, and 10;
each R⁵ is independently selected from C₁₋₃ alkyl, C₂₋₃ alkenyl, and H;
each R⁶ is independently selected from C₁₋₃ alkyl, C₂₋₃ alkenyl, and H;
M and M′ are each independently selected from —C(O)O— and —OC(O)—;
R′ is C₁₋₁₂ alkyl or C₂₋₁₂ alkenyl;
l is selected from 1, 2, 3, 4, and 5; and
m is selected from 5, 6, 7, 8, 9, 10, 11, 12, and 13.

72. The lipid nanoparticle composition of any one of claims 68-70, wherein the ionizable lipid is 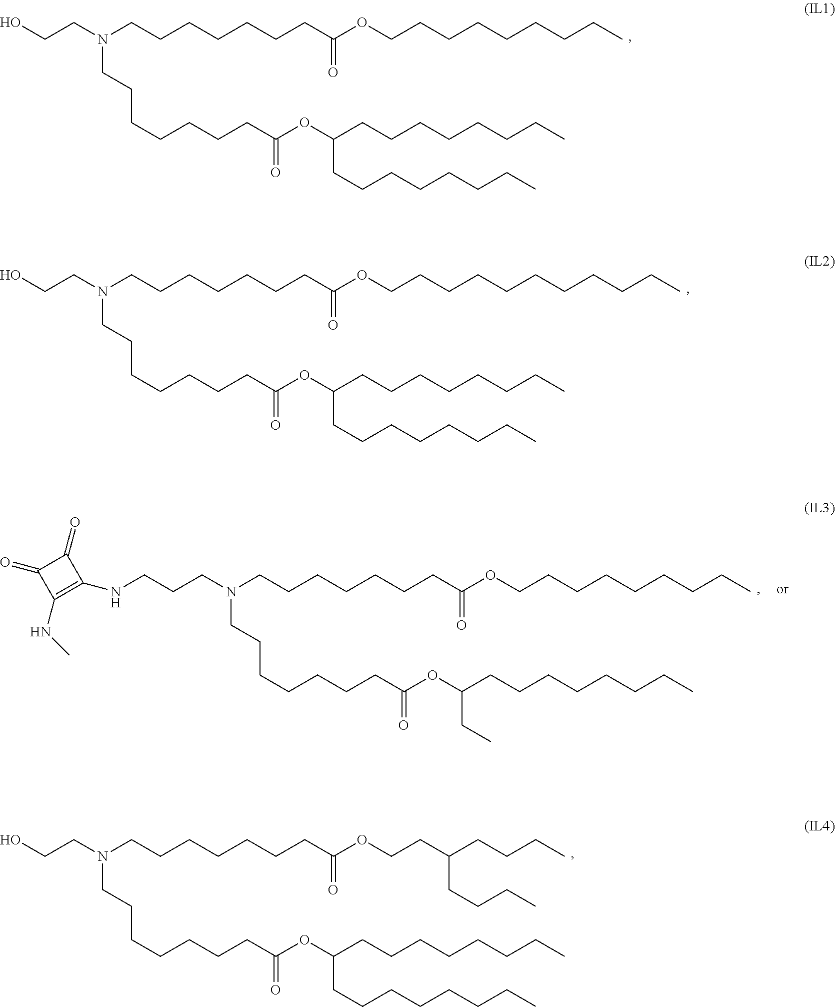 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCCCN(CCO)CCCCCCCC(=O)OC(CCCCCCCC)CCCCCCCC ; CCCCCCCCCOC(=O)CCCCCCCN(CCCCCCCC(=O)OC(CC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]
or an N-oxide or a salt thereof.

73. The lipid nanoparticle composition of any one of claims 68-70, wherein the ionizable lipid is IL1: 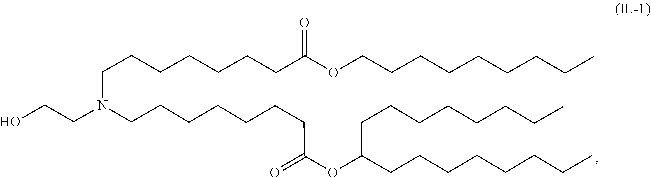 [SMILES: CCCCCCCCCOC(=O)CCCCCCCN(CCO)CCCCCCCC(=O)OC(CCCCCCCC)CCCCCCCC]
or a salt thereof.

74. The lipid nanoparticle composition of any one of claims 68-73, wherein the lipid nanoparticle composition comprises about 30 mol % to about 60 mol % ionizable lipid.

75. The lipid nanoparticle composition of any one of claims 68-74, wherein the phospholipid is a compound of Formula (IV): 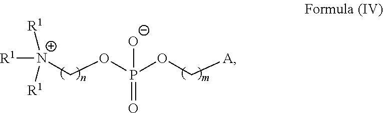
or a salt thereof, wherein:
each R¹ is independently H or optionally substituted alkyl; or optionally two R¹ are joined together with the intervening atoms to form optionally substituted monocyclic cycloalkyl or optionally substituted monocyclic heterocyclyl; or optionally three R¹ are joined together with the intervening atoms to form optionally substituted bicyclic cycloalkyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula: 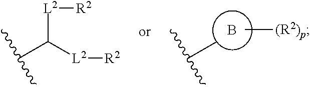
each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2;
provided that the compound is not of the formula: 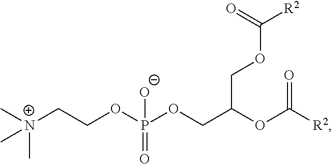
wherein each instance of R² is independently unsubstituted alkyl, unsubstituted alkenyl, or unsubstituted alkynyl.

76. The lipid nanoparticle composition of any one of claims 68-74, wherein the phospholipid is
1,2-distearoyl-sn-glycero-3-phosphocholine (DSPC),
1,2-dioleoyl-sn-glycero-3-phosphoethanolamine (DOPE),
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine (18:0 Diether PC),
1-oleoyl-2-cholesterylhemisuccinoyl-sn-glycero-3-phosphocholine (OChemsPC),
1-hexadecyl-sn-glycero-3-phosphocholine (C16 Lyso PC),
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-diphytanoyl-sn-glycero-3-phosphoethanolamine (ME 16.0 PE),
1,2-diphytanoyl-sn-glycero-3-phosphocholine (4ME 16:0 PC),
1,2-diphytanoyl-sn-glycero-3-phospho-(1′-rac-glycerol) (sodium salt) (4ME 16:0 PG),
1,2-diphytanoyl-sn-glycero-3-phospho-L-serine (sodium salt) (4ME 16:0 PS),
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG), or sphingomyelin.

77. The lipid nanoparticle composition of any one of claims 68-74, wherein the phospholipid is DSPC.

78. The lipid nanoparticle composition of any one of claims 68-77, wherein the lipid nanoparticle composition comprises about 5 mol % to about 15 mol % phospholipid.

79. The lipid nanoparticle composition of any one of claims 68-78, wherein the structural lipid is cholesterol, fecosterol, sitosterol, ergosterol, campesterol, stigmasterol, brassicasterol, tomatidine, tomatine, ursolic acid, alpha-tocopherol, hopanoids, phytosterols, steroids, or mixtures thereof.

80. The lipid nanoparticle composition of any one of claims 68-78, wherein the structural lipid is alpha-tocopherol, cholesterol, 0-sitosterol, or cholesteryl hemisuccinate.

81. The lipid nanoparticle composition of any one of claims 68-80, wherein the lipid nanoparticle composition comprises about 20 mol % to about 60 mol % structural lipid.

82. The lipid nanoparticle composition of any one of claims 68-81, wherein the PEG-lipid is a compound of Formula (V): 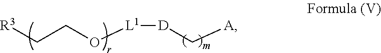
or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted cycloalkylene, optionally substituted heterocyclylene, optionally substituted arylene, optionally substituted heteroarylene, —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
D is a moiety obtained by click chemistry or a moiety cleavable under physiological conditions;
m is 0, 1, 2, 3, 4, 5, 6, 7, 8, 9, or 10;
A is of the formula: 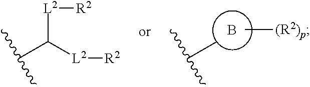
each instance of L² is independently a bond or optionally substituted C₁₋₆ alkylene, wherein one methylene unit of the optionally substituted C₁₋₆ alkylene is optionally replaced with —O—, —N(R^{N})—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted cycloalkylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—;
each instance of R^{N} is independently hydrogen, optionally substituted alkyl, or a nitrogen protecting group;
Ring B is optionally substituted cycloalkyl, optionally substituted heterocyclyl, optionally substituted aryl, or optionally substituted heteroaryl; and
p is 1 or 2.

83. The lipid nanoparticle composition of any one of claims 68-81, wherein the PEG-lipid is PEG-c-DOMG, PEG-DMG, PEG-DLPE, PEG DMPE, PEG-DPPC, PEG-DSPE or PL1:

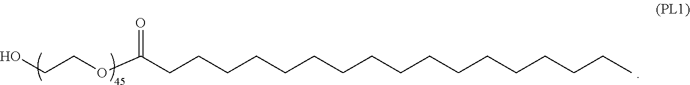

84. The lipid nanoparticle composition of any one of claims 63-78, wherein the lipid nanoparticle composition comprises about 0.1 mol % to about 5.0 mol % PEG-lipid.

85. A pharmaceutical composition comprising the lipid nanoparticle composition of any one of claims 62-79, or a salt thereof, and a pharmaceutically acceptable carrier.

86. The pharmaceutical composition of claim 85, wherein the composition is suitable for inhalation.

87. A method of delivering a payload into a cell comprising contacting the cell with a lipid nanoparticle composition of any one of claims 67-84.

88. The method of claim 87, wherein the cell is an airway epilthelium cell.

89. A method of delivering a therapeutic or prophylactic payload to a patient comprising administering a lipid nanoparticle composition of any one of claims 67-84 to the patient.

90. The method of claim 89, wherein the therapeutic or prophylactic payload is mRNA.

91. The method of claim 89 or 90, wherein the therapeutic or prophylactic payload is delivered to airway epithelium cells in the patient.

92. The method of any one of claims 89-91, wherein the lipid nanoparticle composition is administered by intranasal, intrabronchiol, or pulmonary administration.

93. The method of any one of claims 89-91, wherein the lipid nanoparticle composition is administered by a nebulizer or inhaler.

94. A process of preparing a lipid nanoparticle composition, the process comprising contacting a lipid nanoparticle core with a lipid amine compound of any one of claims 1-66, or a salt thereof.

95. The process of claim 94, wherein the lipid nanoparticle core comprises:
(i) an ionizable lipid,
(ii) a phospholipid,
(iii) a structural lipid, and
(iv) optionally a PEG-lipid.

96. The process of claim 95, wherein the lipid nanoparticle core comprises:
about 40 mol % to about 50 mol % of ionizable lipid;
about 10 mol % to about 12 mol % of phospholipid;
about 37 mol % to about 42 mol % of structural lipid; and
about 0.25 mol % to about 0.75 mol % of PEG-lipid; each with respect to total lipids.

97. The process of claim 95, wherein the lipid nanoparticle core comprises:
about 49 mol % of ionizable lipid;
about 11 mol % to about 12 mol % of phospholipid;
about 39 mol % of structural lipid; and
about 0.5 mol % of PEG-lipid; each with respect to total lipids.

98. The process of any one of claims 95-97, wherein the lipid nanoparticle core further comprises a payload encapsulated for delivery into a cell.

99. The process of any one of claims 94-98, wherein the lipid amine compound is present in a liquid composition.

100. The process of claim 99, wherein the liquid composition comprises a non-ionic excipient.

101. The process of claim 100, wherein the non-ionic excipient is macrogol 15 hydroxystearate (HS 15).

102. A lipid nanoparticle composition prepared by the process of any one of claims 94-101.
